(19) United States
(12) Patent Application Publication
(10) Pub. No.: US 2024/0373745 A1
(43) Pub. Date: Nov. 7, 2024

(54) COMPOUND, ORGANIC ELECTROLUMINESCENT ELEMENT AND ELECTRONIC DEVICE

(71) Applicant: IDEMITSU KOSAN CO., LTD., Tokyo (JP)

(72) Inventors: Kazuki TERADA, Tokyo (JP); Yu KUDO, Tokyo (JP); Kei YOSHIDA, Tokyo (JP); Masato MITANI, Tokyo (JP)

(73) Assignee: IDEMITSU KOSAN CO., LTD., Tokyo (JP)

(21) Appl. No.: 18/647,478

(22) Filed: Apr. 26, 2024

(30) Foreign Application Priority Data
Apr. 27, 2023 (JP) 2023-073699

(51) Int. Cl. H10K 85/60; C07C 13/66; C07D 311/78; C09K 11/06; H10K 50/12

(52) U.S. Cl. CPC H10K 85/622; C07C 13/66; C07D 311/78; C09K 11/06; H10K 85/626; H10K 85/6574; C07C 2603/40; C07C 2603/50; C09K 2211/1007; C09K 2211/1011; C09K 2211/1018; H10K 50/12

(57) ABSTRACT

A compound is represented by a formula (1) below.

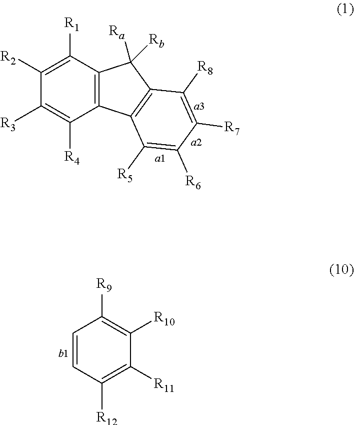

In the formula (1): Rₐ and R_{b} not forming a ring are a substituent or the like; a combination of R₅ and R₆, combination of R₆ and R₇, or combination of R₇ and R⁸ form a ring represented by a formula (10). In this case, positions a1 and b1, positions a2 and b1, or positions a3 and b1 are fused with each other. One or more of R₁ to R₄, R₅ to R₈ not forming a ring, and R₉ to R₁₂ are a substituent Rx. One or more of R₄ to R₆, R₈, R₉ and R₁₂ not being Rx are a substituent Ry, and R₁ to R₁₂ other than Rx and Ry are a hydrogen atom or substituent. At least one hetero atom of when the substituent is a heterocyclic group only includes one or more of oxygen, sulfur and silicon.

The entire disclosure of Japanese Patent Application No. 2023-073699, filed Apr. 27, 2023, is expressly incorporated by reference herein.

### TECHNICAL FIELD

The present invention relates to a compound, an organic electroluminescence device, and an electronic device.

### BACKGROUND ART

An organic electroluminescence device (hereinafter, occasionally referred to as “organic EL device”) has found its application in a full-color display for mobile phones, televisions, and the like. When voltage is applied to an organic EL device, holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

The performance of the organic EL device is evaluable in terms of, for instance, luminance, emission wavelength, chromaticity, luminous efficiency, drive voltage, and lifetime. For instance, studies for improving performance of an organic EL device have been made in Literatures 1 (International Publication No. WO2023/017704).

### SUMMARY OF THE INVENTION

An object of the invention is to provide a compound that improves luminous efficiency of an organic electroluminescence device, an organic electroluminescence device containing the compound, and an electronic device including the organic electroluminescence device.

According to an aspect of the invention, there is provided a compound represented by a formula (1) below.

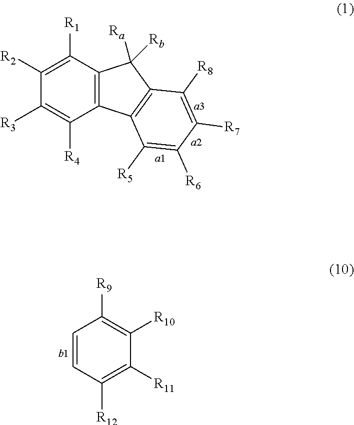

In the formula (1):
a combination of Rₐ and R_{b} are mutually bonded to form a substituted or unsubstituted monocyclic ring, mutually bonded to form a substituted or unsubstituted fused ring, or not mutually bonded;
Rₐ and R_{b} forming neither the substituted or unsubstituted monocyclic ring nor the substituted or unsubstituted fused ring are each independently a substituted or unsubstituted alkyl group having 1 to 4 carbon atoms, a substituted or unsubstituted aryl group having 6 to 50 ring carbon atoms, or a substituted or unsubstituted heterocyclic group having 5 to 50 ring atoms;
the heterocyclic group as Rₐ and R_{b} is a heterocyclic group only including at least one atom selected from the group consisting of an oxygen atom, a sulfur atom, and a silicon atom as a hetero atom;
any one combination of a combination of R₅ and R₆, a combination of R₆ and R₇, and a combination of R₇ and R₈ are mutually bonded to form a ring represented by the formula (10);
when a combination of R₅ and R₆ form a ring represented by the formula (10), fused positions are a1 and b1;
when a combination of R₆ and R₇ form a ring represented by the formula (10), fused positions are a2 and b1;
when a combination of R₇ and R₈ form a ring represented by the formula (10), fused positions are a3 and b1;
at least one of R₁ to R₄, R₅ to R₆ not forming a ring represented by the formula (10), or R₉ to R₁₂ in the formula (10) is a substituent Rx;
the substituent Rx is each independently a substituted or unsubstituted aryl group fused with four or more six-membered rings, or a substituted or unsubstituted heterocyclic ring group fused with four or more rings;
the heterocyclic group as the substituent Rx is a heterocyclic group only including at least one atom selected from the group consisting of an oxygen atom, a sulfur atom, and a silicon atom as a hetero atom;
at least one of R₄, R₉, or R₁₂ not being the substituent Rx, or R₅, R₆, or R₈ neither forming a ring represented by the formula (10) nor being the substituent Rx is a substituent Ry,
the substituent Ry is each independently a substituted or unsubstituted phenyl group, a substituted or unsubstituted naphthyl group, a substituted or unsubstituted phenanthryl group, a substituted or unsubstituted dibenzofuranyl group, or a substituted or an unsubstituted dibenzothienyl group;
when the substituent Ry is a substituted 1-naphthyl group, a carbon atom at a position 4 of the 1-naphthyl group is unsubstituted;
R₁ to R₄ and R₉ to R₁₂ being neither the substituent Rx nor the substituent Ry, and R₅ to R₈ neither forming a ring represented by the formula (10) nor being the substituent Rx nor being the substituent Ry are each independently a hydrogen atom, a substituted or unsubstituted alkyl group having 1 to 50 carbon atoms, a substituted or unsubstituted cycloalkyl group having 3 to 50 carbon atoms, a substituted or unsubstituted aryl group having 6 to 50 ring carbon atoms, or a substituted or unsubstituted heterocyclic group having 5 to 50 ring atoms;
the heterocyclic group as R₁ to R₁₂ being neither the substituent Rx nor the substituent Ry is a heterocyclic group only including at least one atom selected from the group consisting of an oxygen atom, a sulfur atom, and a silicon atom as a hetero atom; and
a combination of adjacent two or more of R₁ to R₄ and R₉ to R₁₂ being neither the substituent Rx nor the substituent Ry, and R₅ to R₈ neither forming a ring represented by the formula (10) nor being the substituent Rx nor being the substituent Ry are not bonded to each other to form no ring.

According to another aspect of the invention, there is provided an organic electroluminescence device containing the compound according to the above aspect of the invention as a first compound.

According to still another aspect of the invention, there is provided an electronic device including the organic electroluminescence device according to the above aspect of the invention.

According to the above aspects of the invention, there can be provided a compound that improves luminous efficiency of an organic electroluminescence device, an organic electroluminescence device containing the compound, and an electronic device including the organic electroluminescence device.

### BRIEF DESCRIPTION OF DRAWINGS

FIG. 1 schematically depicts an exemplary arrangement of an organic electroluminescence device according to an exemplary embodiment of the invention.

FIG. 2 schematically depicts another exemplary arrangement of the organic electroluminescence device according to the exemplary embodiment of the invention.

### DETAILED DESCRIPTION

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, crosslinking compound, carbon ring compound, and heterocyclic compound) in which the atoms are bonded to each other to form the ring.

When the ring is substituted by a substituent(s), carbon atom(s) contained in the substituent(s) is not counted in the ring carbon atoms. Unless specifically described, the same applies to the “ring carbon atoms” described later. For instance, a benzene ring has 6 ring carbon atoms, a naphthalene ring has 10 ring carbon atoms, a pyridine pyridine ring has 5 ring carbon atoms, and a furan ring 4 ring carbon atoms. For instance, a 9,9-diphenylfluorenyl group has 13 ring carbon atoms and 9,9′-spirobifluorenyl group has 25 ring carbon atoms.

When a benzene ring is substituted by a substituent, e.g., an alkyl group, the number of carbon atoms of the alkyl group is not counted in the number of the ring carbon atoms of the benzene ring. Accordingly, the benzene ring substituted by an alkyl group has 6 ring carbon atoms. When a naphthalene ring is substituted by a substituent in a form of, for instance, an alkyl group, the number of carbon atoms of the alkyl group is not counted in the number of the ring carbon atoms of the naphthalene ring. Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly). Atom(s) not forming the ring (e.g., hydrogen atom(s) for saturating the valence of the atom which forms the ring) and atom(s) in a substituent by which the ring is substituted are not counted as the ring atoms. Unless otherwise specified, the same applies to the “ring atoms” described later. For instance, a pyridine ring has 6 ring atoms, a quinazoline ring has 10 ring atoms, and a furan ring has 5 ring atoms. For instance, the number of hydrogen atom(s) bonded to a pyridine ring or the number of atoms forming a substituent is not counted as ring atoms of the pyridine ring. Accordingly, a pyridine ring bonded to a hydrogen atom(s) or a substituent(s) has 6 ring atoms. For instance, the hydrogen atom(s) bonded to carbon atom(s) of a quinazoline ring or the atoms forming a substituent are not counted as the quinazoline ring atoms. Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group. Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group. Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s). The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent. Similarly, the term “substituted” used in a “BB group substituted by AA group” means that at least one hydrogen atom in the BB group is substituted with the AA group.

### Substituents Mentioned Herein

Substituents mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B). Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.” A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent. Examples of the “substituted aryl group” include a group derived by substituting at least one hydrogen atom in the “unsubstituted aryl group” in the specific example group G1A below with a substituent, and examples of the substituted aryl group in the specific example group G1B below. It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below with a substituent, and a group derived by further substituting a hydrogen atom of a substituent of the “substituted aryl group” in the specific example group G1B below with a substituent.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

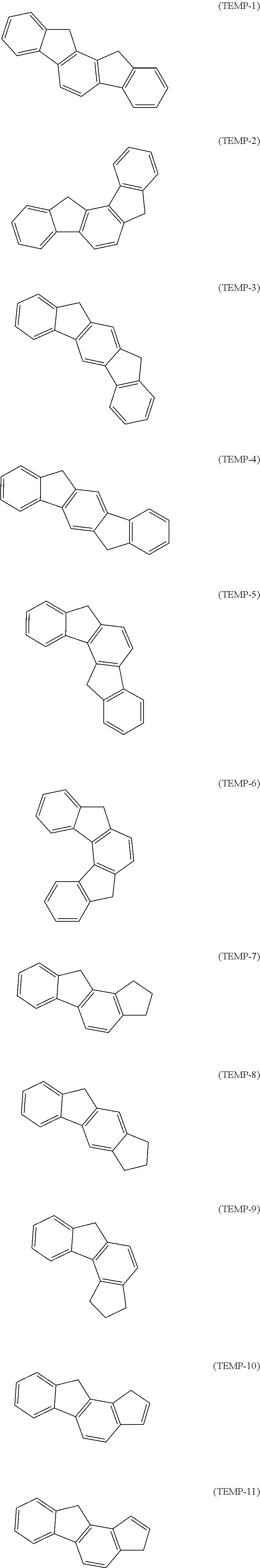

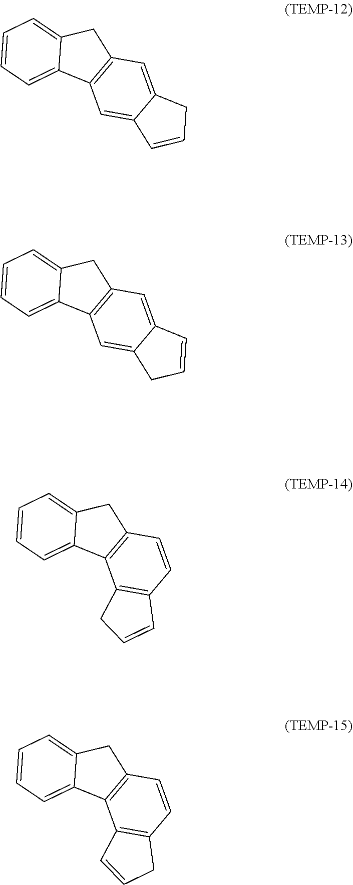

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethylphenyl group, 9,9-dimethylfluorenyl group, 9,9-diphenylfluorenyl group, 9,9-bis(4-methylphenyl) fluorenyl group, 9,9-bis(4-isopropylphenyl) fluorenyl group, 9,9-bis(4-t-butylphenyl) fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms. Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B). (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent. Specific examples of the “substituted heterocyclic group” include examples of a group derived by substituting at least one hydrogen atom in the “unsubstituted heterocyclic group” in the specific example group G2A below with a substituent, and examples of the substituted heterocyclic group in the specific example group G2B below. It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below with a substituent, and a group derived by further substituting a hydrogen atom of a substituent of the “substituted heterocyclic group” in the specific example group G2B below with a substituent.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4), with a substituent, derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).
   Monovalent Heterocyclic Groups Derived by Removing One Hydrogen Atom from Cyclic Structures Represented by Formulae (TEMP-16) to (TEMP-33) (Specific Example Group G2A4):

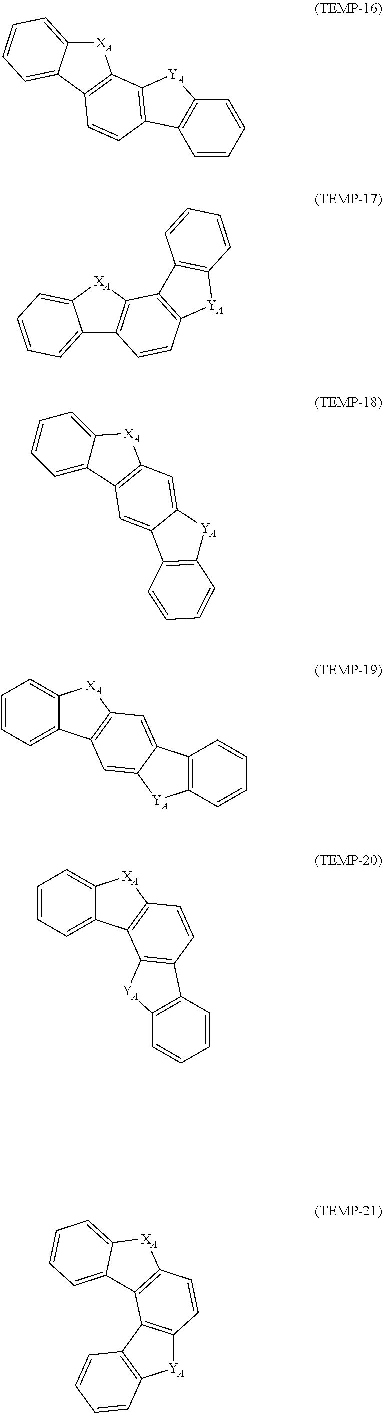

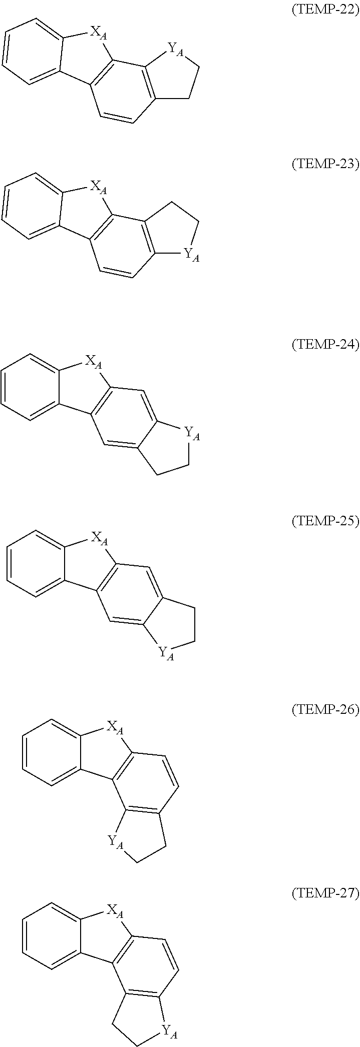

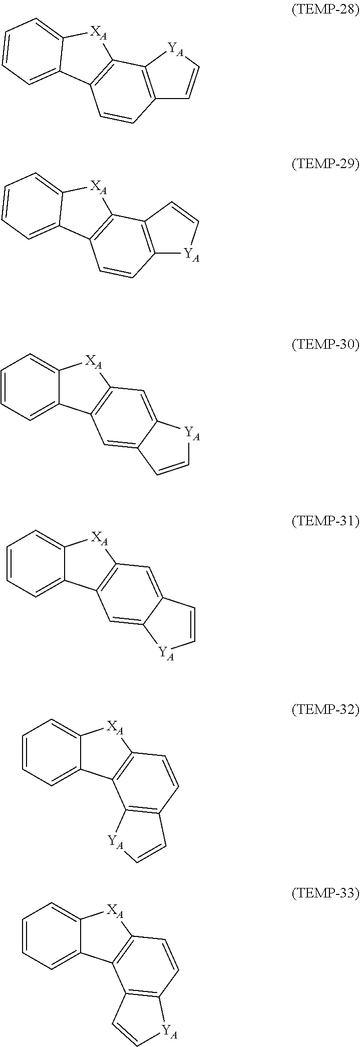

In the formulae (TEMP-16) to (TEMP-33), X_{A} and Y_{A} are each independently an oxygen atom, a sulfur atom, NH or CH₂, with a proviso that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

a (9-phenyl) carbazolyl group, (9-biphenylyl) carbazolyl group, (9-phenyl)phenylcarbazolyl group, (9-naphthyl) carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

Substituted Heterocyclic Groups Including Oxygen Atom (Specific Example Group G2B2):
phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, methyldibenzothiophenyl group, t-butyldibenzothiophenyl group, and monovalent residue of spiro[9H-thioxanthene-9,9′-[9H]fluorene].
   Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X_{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below. (Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”) A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent. Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below. Herein, the alkyl group for the “unsubstituted alkyl group” refers to a chain alkyl group. Accordingly, the “unsubstituted alkyl group” include linear “unsubstituted alkyl group” and branched “unsubstituted alkyl group.” It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B with a substituent, and a group derived by further substituting a hydrogen atom of a substituent of the “substituted alkyl group” in the specific example group G3B with a substituent.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B). (Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”) A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent. Specific examples of the “substituted alkenyl group” include an “unsubstituted alkenyl group” (specific example group G4A) substituted by a substituent, and examples of the substituted alkenyl group (specific example group G4B) below. It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below. (Herein, an unsubstituted alkynyl group refers to an “unsubstituted alkynyl group” in a “substituted or unsubstituted alkynyl group.”) A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent. Specific examples of the “substituted alkynyl group” include a group derived by substituting at least one hydrogen atom of the “unsubstituted alkynyl group” (specific example group G5A) below with a substituent.

Unsubstituted Alkynyl Group (Specific Example Group G5A): ethynyl group

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B). (Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”) A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent. Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below. It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

a 4-methylcyclohexyl group.
   Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6); where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include:—O(G1); —O(G2); —O(G3); and —O(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

Group Represented by —S—(R₉₀₅)

Specific examples (specific example group G9) of a group represented herein by —S—(R₉₀₅) include: —S(G1); —S(G2); —S(G3); and —S(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms. An “unsubstituted fluoroalkyl group” has, unless otherwise specified herein, 1 to 50, preferably 1 to 30, more preferably 1 to 18 carbon atoms. The “substituted fluoroalkyl group” refers to a group derived by substituting at least one hydrogen atom in a “fluoroalkyl group” with a substituent. It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent. Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms. An “unsubstituted haloalkyl group” has, unless otherwise specified herein, 1 to 50, preferably 1 to 30, and more preferably 1 to 18 carbon atoms. The “substituted haloalkyl group” refers to a group derived by substituting at least one hydrogen atom in a “haloalkyl group” with a substituent. It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent. Specific examples of the “unsubstituted haloalkyl group” include a group derived by substituting at least one hydrogen atom of the “alkyl group” (specific example group G3) with a halogen atom. The haloalkyl group is occasionally referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3. An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3. An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1. An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1. An “unsubstituted arylthio group” has, unless otherwise specified herein, 6 to 50, preferably 6 to 30, more preferably 6 to 18 ring carbon atoms.

### Substituted or Unsubstituted Trialkylsilyl Group

Specific examples of a “trialkylsilyl group” mentioned herein include a group represented by —Si(G3)(G3)(G3), G3 being the “substituted or unsubstituted alkyl group” in the specific example group G3. A plurality of G3 in —Si(G3)(G3)(G3) are mutually the same or different. Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by —(G3)-(G1), G3 being the “substituted or unsubstituted alkyl group” in the specific example group G3, G1 being the “substituted or unsubstituted aryl group” in the specific example group G1. Accordingly, the “aralkyl group” is a group derived by substituting a hydrogen atom of the “alkyl group” with a substituent in a form of the “aryl group,” which is an example of the “substituted alkyl group.” An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl) carbazolyl group ((9-phenyl) carbazole-1-yl group, (9-phenyl) carbazole-2-yl group, (9-phenyl) carbazole-3-yl group, or (9-phenyl) carbazole-4-yl group), (9-biphenylyl) carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

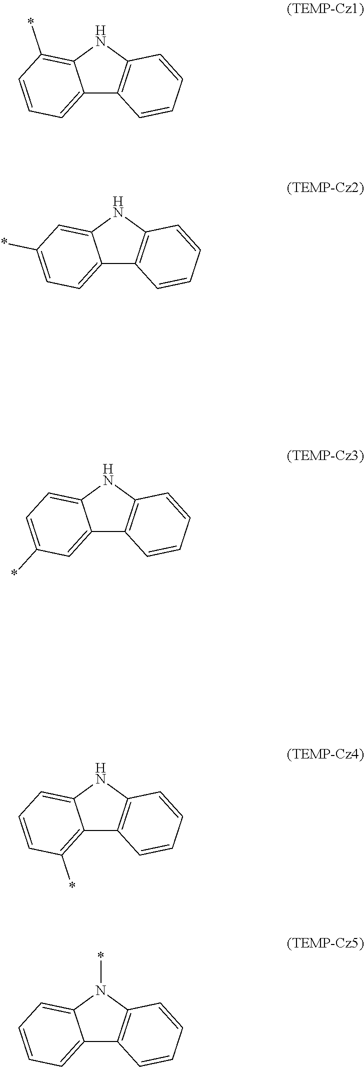 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

The (9-phenyl) carbazolyl group mentioned herein is, unless otherwise specified herein, specifically a group represented by one of formulae below.

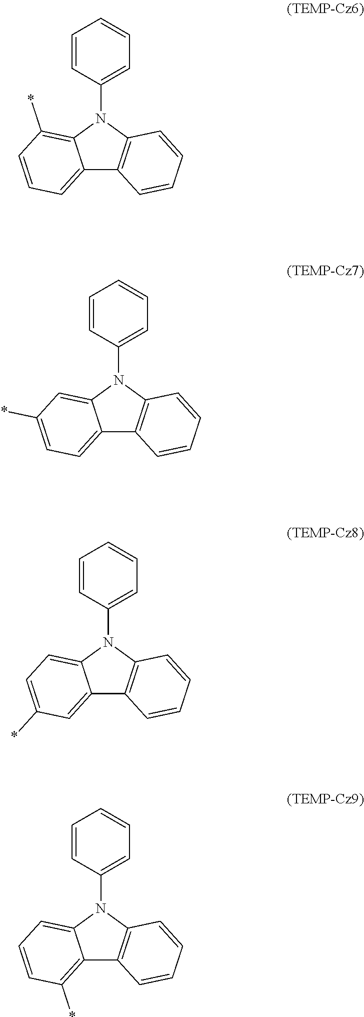 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

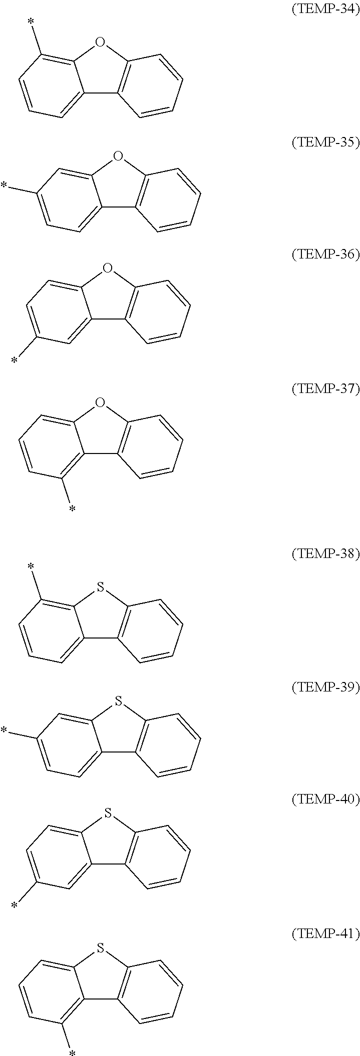

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.” Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocyclic ring of the “substituted or unsubstituted heterocyclic group.” Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.” Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae 10 (TEMP-42) to (TEMP-68) below.

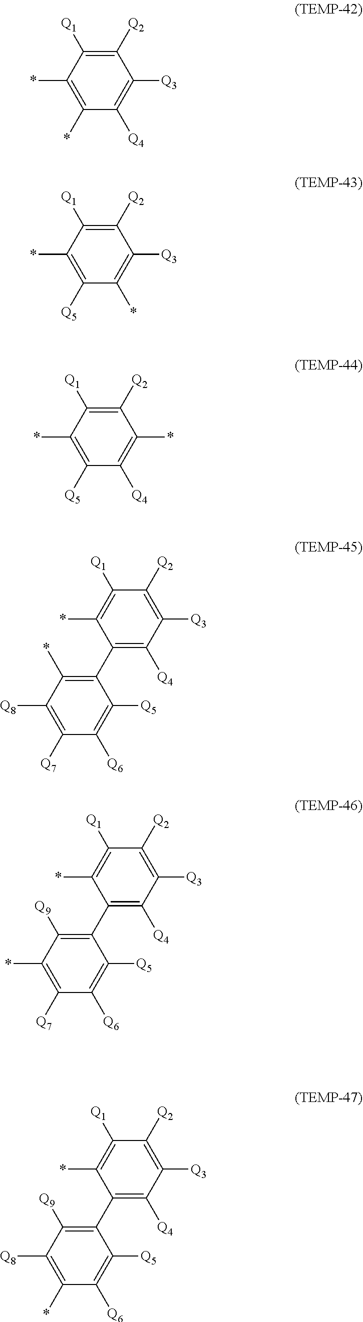

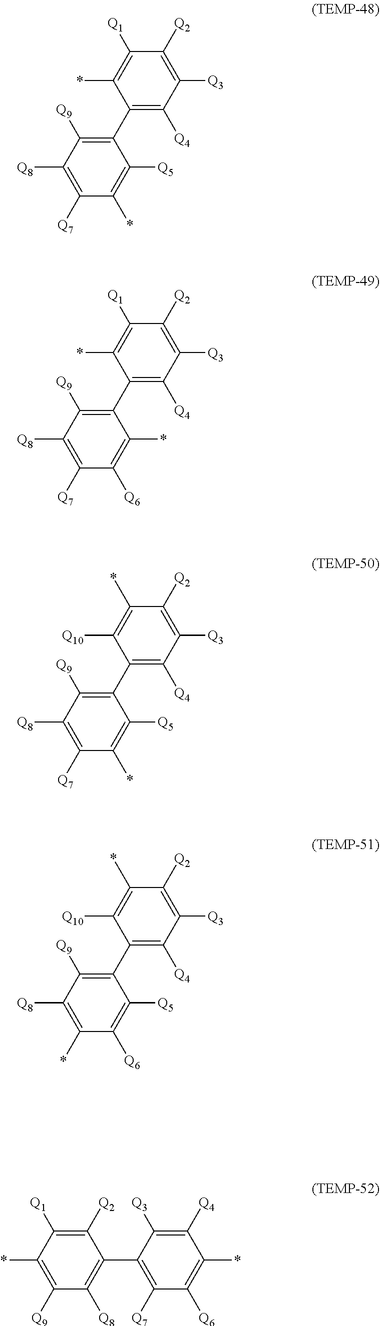

In the formulae (TEMP-42) to (TEMP-52), Q₁ to Q₁₀ are each independently a hydrogen atom or a substituent.

In the formulae (TEMP-42) to (TEMP-52), * represents a bonding position.

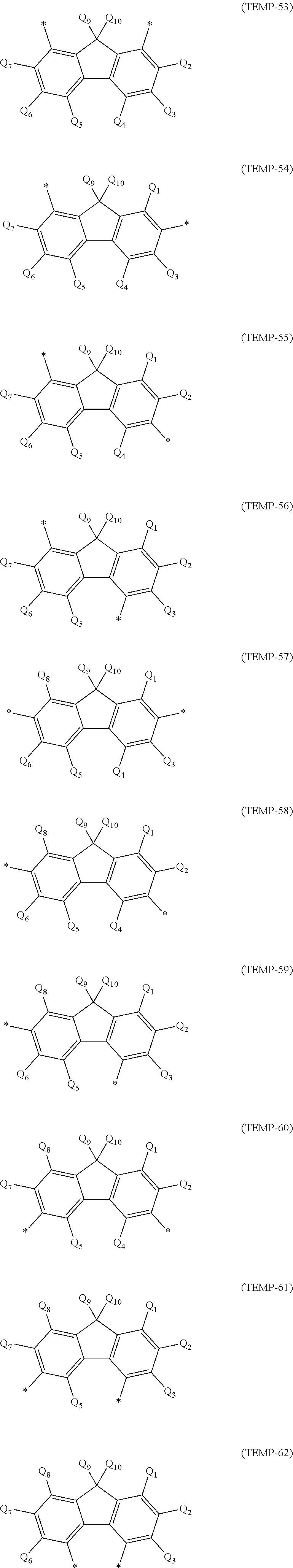

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

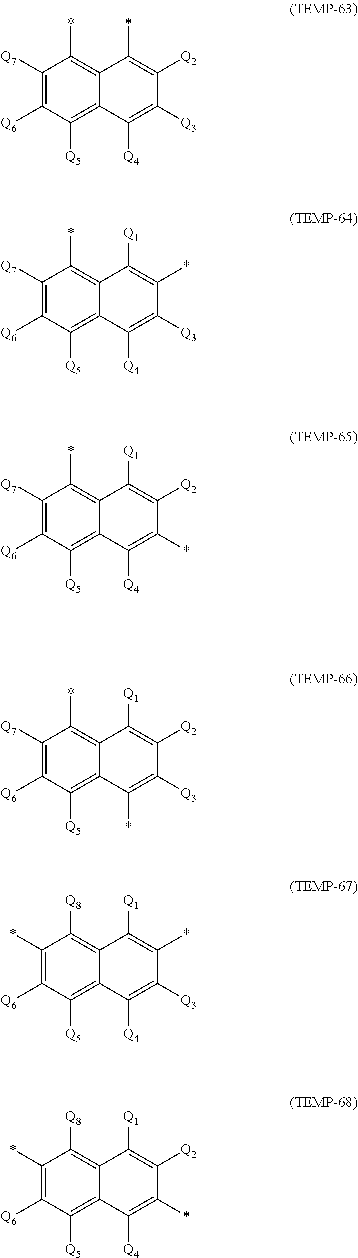

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

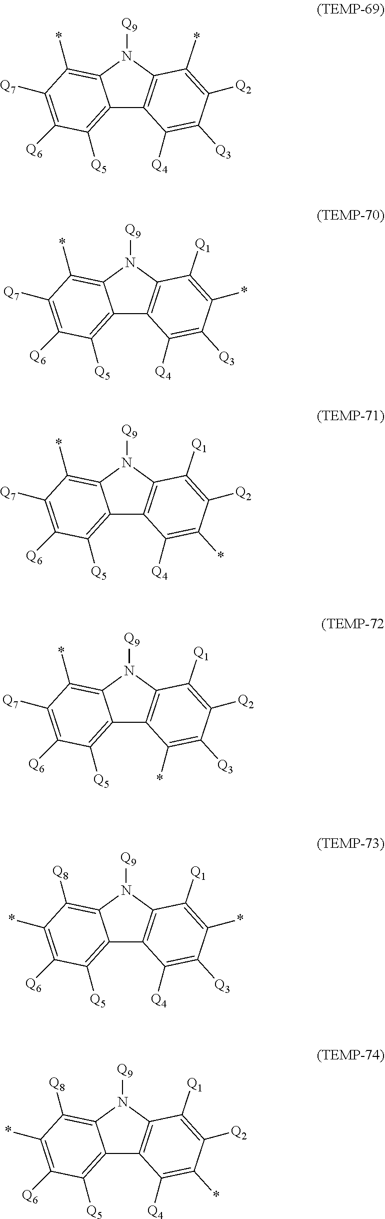

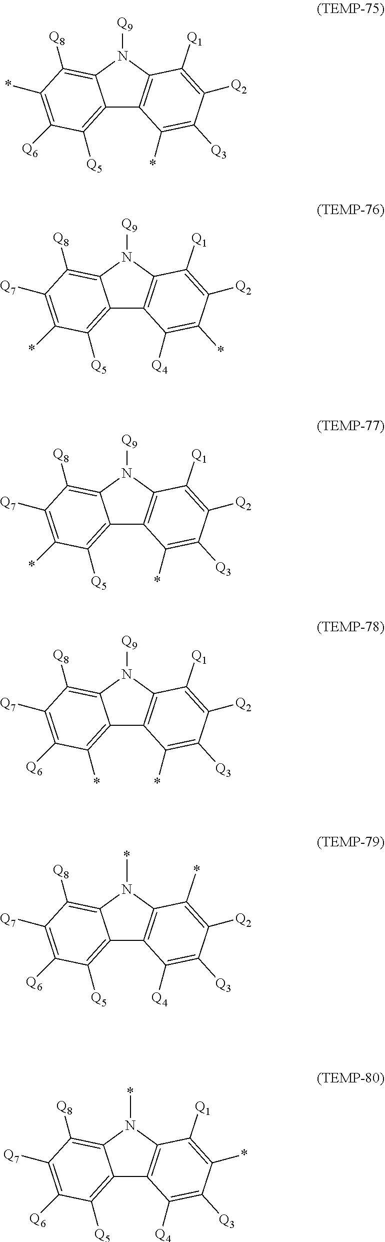

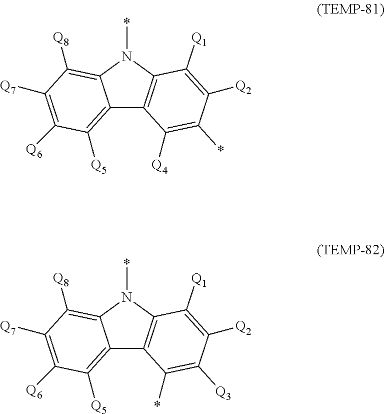

In the formulae (TEMP-69) to (TEMP-82), Q₁ to Q₉ are each independently a hydrogen atom or a substituent.

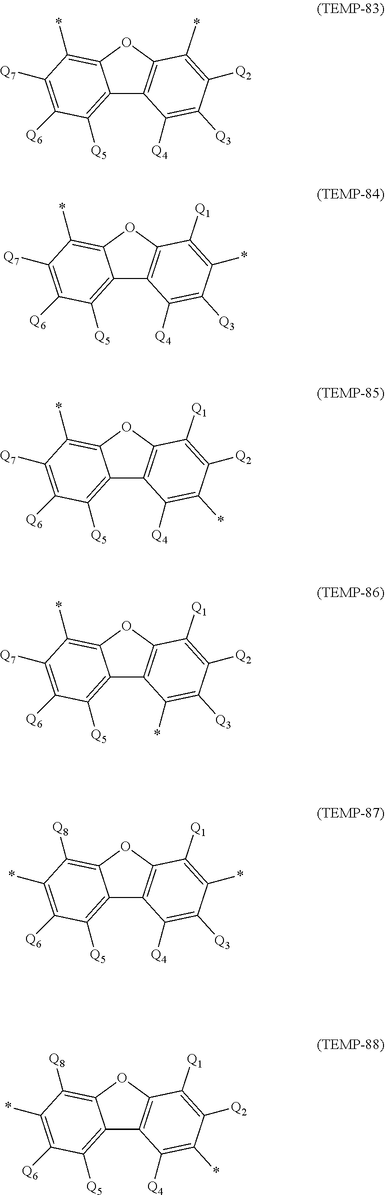

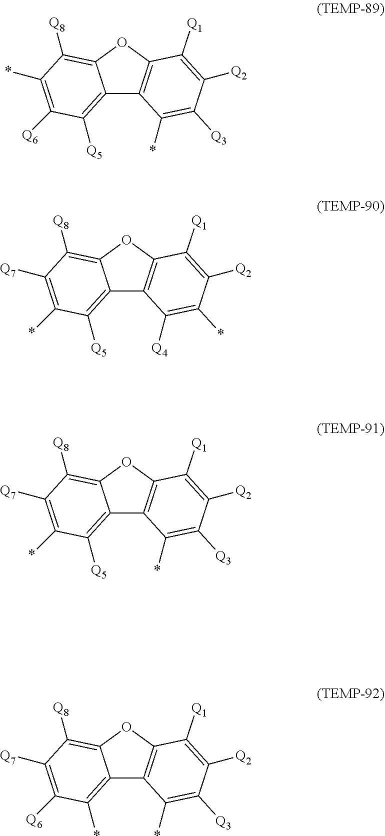

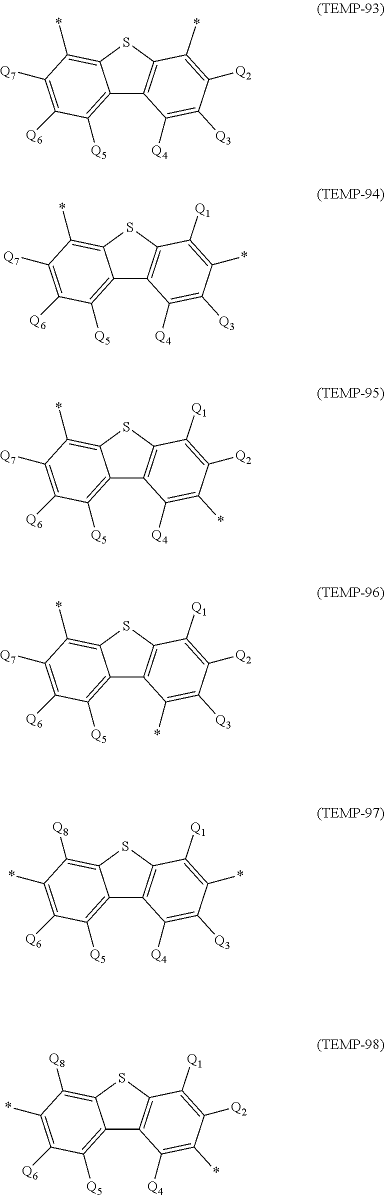

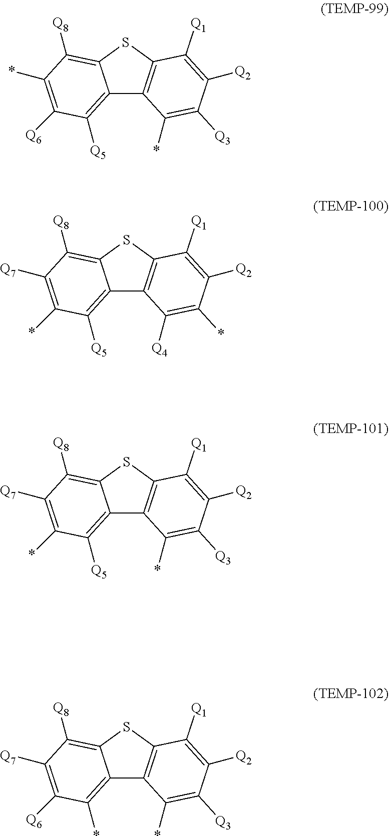

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (these instances will be sometimes collectively referred to as an instance of “bonded to form a ring” hereinafter) will be described below. An anthracene compound having a basic skeleton in a form of an anthracene ring and represented by a formula (TEMP-103) below will be used as an example for the description.

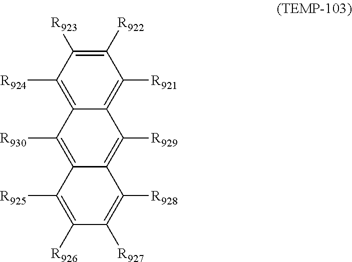

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings. For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

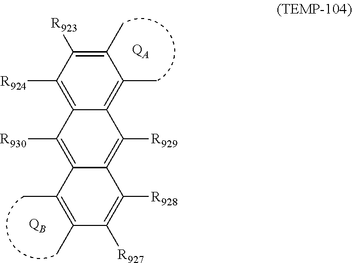

The instance where the “combination of adjacent two or more” form a ring means not only an instance where the “two” adjacent components are bonded but also an instance where adjacent “three or more” are bonded. For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below. In the formula (TEMP-105) below, the ring Q_{A} and the ring Q_{C} share R₉₂₂.

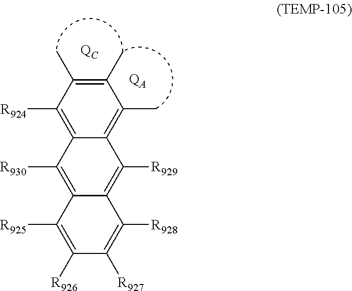

The formed “monocyclic ring” or “fused ring” may be, in terms of the formed ring in itself, a saturated ring or an unsaturated ring. When the “combination of adjacent two” form a “monocyclic ring” or a “fused ring,” the “monocyclic ring” or “fused ring” may be a saturated ring or an unsaturated ring. For instance, the ring Q_{A} and the ring Q_{B} formed in the formula (TEMP-104) are each independently a “monocyclic ring” or a “fused ring.” Further, the ring Q_{A} and the ring Q_{C} formed in the formula (TEMP-105) are each a “fused ring.” The ring Q_{A} and the ring Q_{C} in the formula (TEMP-105) are fused to form a fused ring. When the ring Q_{A} in the formula (TEMP-104) is a benzene ring, the ring Q_{A} is a monocyclic ring. When the ring Q_{A} in the formula (TEMP-104) is a naphthalene ring, the ring Q_{A} is a fused ring.

The “unsaturated ring” represents an aromatic hydrocarbon ring or an aromatic heterocycle. The “saturated ring” represents an aliphatic hydrocarbon ring or a non-aromatic heterocycle.

Specific examples of the aromatic hydrocarbon ring include a ring formed by terminating a bond of a group in the specific examples of the specific example group G1 with a hydrogen atom.

Specific examples of the aromatic heterocyclic ring include a ring formed by terminating a bond of an aromatic heterocyclic group in the specific examples of the specific example group G2 with a hydrogen atom.

Specific examples of the aliphatic hydrocarbon ring include a ring formed by terminating a bond of a group in the specific examples of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms. For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms. Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom. A bond of the optional atom (e.g. a carbon atom and a nitrogen atom) not forming a ring may be terminated by a hydrogen atom or the like or may be substituted by an “optional substituent” described later. When the ring includes any other optional element than the carbon atom, the resultant ring is a heterocyclic ring.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.” When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (sometimes referred to as an “optional substituent” hereinafter), is for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;
R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent. Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

### First Exemplary Embodiment

### Compounds

A compound according to a first exemplary embodiment is represented by a formula (1) below.

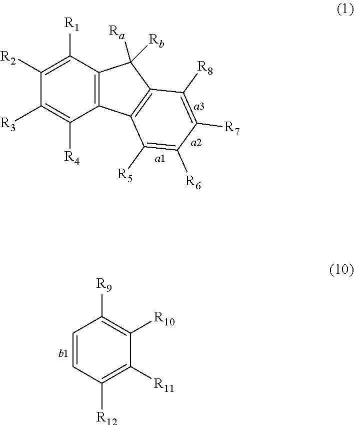

In the formula (1):
a combination of Rₐ and R_{b} are mutually bonded to form a substituted or unsubstituted monocyclic ring, mutually bonded to form a substituted or unsubstituted fused ring, or not mutually bonded;
Rₐ and R_{b} forming neither the substituted or unsubstituted monocyclic ring nor the substituted or unsubstituted fused ring are each independently a substituted or unsubstituted alkyl group having 1 to 4 carbon atoms, a substituted or unsubstituted aryl group having 6 to 50 ring carbon atoms, or a substituted or unsubstituted heterocyclic group having 5 to 50 ring atoms;
the heterocyclic group as Rₐ and R_{b} is a heterocyclic group only including at least one atom selected from the group consisting of an oxygen atom, a sulfur atom, and a silicon atom as a hetero atom;
any one combination of a combination of R₅ and R₆, a combination of R₆ and R₇, and a combination of R₇ and R₈ are mutually bonded to form a ring represented by the formula (10);
when a combination of R₅ and R₆ form a ring represented by the formula (10), fused positions are a1 and b1;
when a combination of R₆ and R₇ form a ring represented by the formula (10), fused positions are a2 and b1;
when a combination of R₇ and R₈ form a ring represented by the formula (10), fused positions are a3 and b1;
at least one of R₁ to R₄, R₅ to R₈ not forming a ring represented by the formula (10), or R₉ to R₁₂ in the formula (10) is a substituent Rx;
the substituent Rx is each independently a substituted or unsubstituted aryl group fused with four or more six-membered rings, or a substituted or unsubstituted heterocyclic ring group fused with four or more rings;
the heterocyclic group as the substituent Rx is a heterocyclic group only including at least one atom selected from the group consisting of an oxygen atom, a sulfur atom, and a silicon atom as a hetero atom;
at least one of R₄, R₉, or R₁₂ not being the substituent Rx, or R₅, R₆, or R₈ neither forming a ring represented by the formula (10) nor being the substituent Rx is a substituent Ry,
the substituent Ry is each independently a substituted or unsubstituted phenyl group, a substituted or unsubstituted naphthyl group, a substituted or unsubstituted phenanthryl group, a substituted or unsubstituted dibenzofuranyl group, or a substituted or an unsubstituted dibenzothienyl group;
when the substituent Ry is a substituted 1-naphthyl group, a carbon atom at a position 4 of the 1-naphthyl group is unsubstituted;
R₁ to R₄ and R₉ to R₁₂ being neither the substituent Rx nor the substituent Ry, and R₅ to R₈ neither forming a ring represented by the formula (10) nor being the substituent Rx nor being the substituent Ry are each independently a hydrogen atom, a substituted or unsubstituted alkyl group having 1 to 50 carbon atoms, a substituted or unsubstituted cycloalkyl group having 3 to 50 carbon atoms, a substituted or unsubstituted aryl group having 6 to 50 ring carbon atoms, or a substituted or unsubstituted heterocyclic group having 5 to 50 ring atoms;
the heterocyclic group as R₁ to R₁₂ being neither the substituent Rx nor the substituent Ry is a heterocyclic group only including at least one atom selected from the group consisting of an oxygen atom, a sulfur atom, and a silicon atom as a hetero atom; and
a combination of adjacent two or more of R₁ to R₄ and R₉ to R₁₂ being neither the substituent Rx nor the substituent Ry, and R₅ to R₈ neither forming a ring represented by the formula (10) nor being the substituent Rx nor being the substituent Ry are not bonded to each other to form no ring.

In the compound according to the exemplary embodiment, that (a) a heterocyclic group as Rₐ and R_{b}, (b) a heterocyclic group as a substituent Rx, and (c) a heterocyclic group as R₁ to R₁₂ being neither a substituent Rx nor a substituent Ry only include at least one atom selected from the group consisting of an oxygen atom, a sulfur atom, and a silicon atom as a hetero atom means that the hetero atom may be plural as long as the hetero atom is an oxygen atom, a sulfur atom, or a silicon atom and that the hetero atoms as ring atoms of the heterocyclic group in the above (a) to (c) includes only one or only two or more of an oxygen atom, a sulfur atom, and a silicon atom. It should be noted that the hetero atoms as ring atoms of the heterocyclic group in the above (a) to (c) include no nitrogen atom.

In the compound according to the exemplary embodiment, the heterocyclic group in the above (a) to (c) is a monocyclic group or a fused-ring group.

In the compound according to the exemplary embodiment, the heterocyclic group in the above (a) to (c) is an aromatic heterocyclic group or a non-aromatic heterocyclic group.

In the compound according to the exemplary embodiment, specific examples (the specific example group G2) of the “substituted or unsubstituted heterocyclic group” include a heterocyclic group only including at least one atom selected from the group consisting of an oxygen atom, a sulfur atom, and a silicon atom as a hetero atom, among the “unsubstituted heterocyclic group” in the specific example group G2A, the substituted heterocyclic group in the specific example group G2B, and the like. (Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”) A simply termed “heterocyclic group” herein includes both of an “unsubstituted heterocyclic group” and a “substituted heterocyclic group.”

In the compound according to the exemplary embodiment, the “substituted heterocyclic group” refers to a group derived by substituting at least one hydrogen atom in an “unsubstituted heterocyclic group” with a substituent. Specific examples of the “substituted heterocyclic group” include a heterocyclic group only including at least one atom selected from the group consisting of an oxygen atom, a sulfur atom, and a silicon atom as a hetero atom, in examples of a group derived by substituting at least one hydrogen atom in the “unsubstituted heterocyclic group” in the specific example group G2A and examples of the substituted heterocyclic group in the specific example group G2B. It should be noted that the examples of the “unsubstituted heterocyclic group” and the “substituted heterocyclic group” mentioned herein are merely exemplary, and the examples of the “substituted heterocyclic group” in the compound according to the exemplary embodiment also include a heterocyclic group only including at least one atom selected from the group consisting of an oxygen atom, a sulfur atom, and a silicon atom as a hetero atom, among a group derived by further substituting a hydrogen atom bonded to a ring atom of a skeleton of a “substituted heterocyclic group” in the specific example group G2B with a substituent, and a group derived by further substituting a hydrogen atom of a substituent of the “substituted heterocyclic group” in the specific example group G2B with a substituent.

In the compound according to the exemplary embodiment, the specific example group G2A includes heterocyclic groups (i), (ii), and (iii) as follows:
(i) a group only including an oxygen atom as a hetero atom and a group only including an oxygen atom and at least one of a sulfur atom or a silicon atom as a hetero atom, among the unsubstituted heterocyclic group including an oxygen atom (the specific example group G2A2);
(ii) a group only including a sulfur atom as a hetero atom and a group only including a sulfur atom and at least one of an oxygen atom or a silicon atom as a hetero atom, among the unsubstituted heterocyclic group including a sulfur atom (specific example group G2A3); and
(iii) a group including only one or only two of an oxygen atom, a sulfur atom, and a silicon atom as a hetero atom, among the monovalent heterocyclic groups derived by removing one hydrogen atom from the cyclic structures represented by the formulae (TEMP-16) to (TEMP-33) (specific example group G2A4).

In the compound according to the exemplary embodiment, at least one of R₂ or R₃ is preferably the substituent Rx.

In the compound according to the exemplary embodiment, at least one of R₄, R₅, R₉, or R₁₂ is each preferably the substituent Ry.

In the compound according to the exemplary embodiment, a combination of Rₐ and R_{b} are preferably not bonded to each other to form no ring.

In the compound according to the exemplary embodiment, a combination of Rₐ and R_{b} are also preferably bonded to each other to form an unsubstituted fused ring.

In the compound according to the exemplary embodiment, Rₐ and R_{b} are also preferably each independently an unsubstituted phenyl group.

In the compound according to the exemplary embodiment, Rₐ and R_{b} are preferably each independently a substituted or unsubstituted alkyl group having 1 to 4 carbon atoms.

In the compound according to the exemplary embodiment, Rₐ and R_{b} are also preferably each independently a methyl group or an ethyl group.

In the compound according to the exemplary embodiment, it is also preferable that the substituent Rx is each independently a substituted or unsubstituted pyrenyl group, a substituted or unsubstituted benzanthryl group, a substituted or unsubstituted benzoxanthenyl group, or a substituted or unsubstituted naphthobenzofuranyl group, the pyrenyl group is represented by a formula (Rx-1) below, the benzanthryl group is represented by a formula (Rx-2) below, the benzoxanthenyl group is represented by a formula (Rx-3) below, and the naphthobenzofuranyl group is represented by a formula (Rx-4) below.

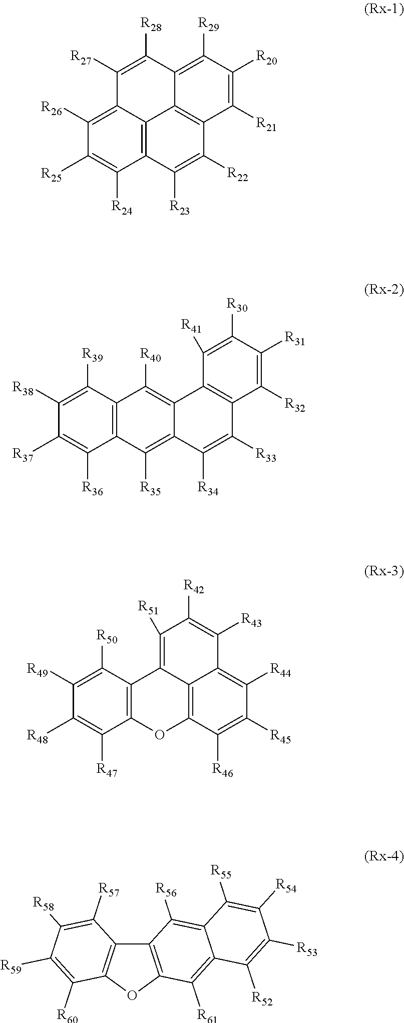

In the formula (Rx-1), one of R₂₀ to R₂₉ is a bond of a substituent Rx, and R₂₀ to R₂₉ other than the bond are each independently a hydrogen atom or a substituent.

In the formula (Rx-2), one of R₃₀ to R₄₁ is a bond of a substituent Rx, and R₃₀ to R₄₁ other than the bond are each independently a hydrogen atom or a substituent.

In the formula (Rx-3), one of R₄₂ to R₅₁ is a bond of a substituent Rx, and R₄₂ to R₅₁ other than the bond are each independently a hydrogen atom or a substituent.

In the formula (Rx-4), one of R₅₂ to R₆₁ is a bond of a substituent Rx, and R₅₂ to R₆₁ other than the bond are each independently a hydrogen atom or a substituent.

In the groups represented by the formulae (Rx-1) to (Rx-4), the substituents as R₂₀ to R₂₉ other than the bond, R₃₀ to R₄₁ other than the bond, R₄₂ to R₅₁ other than the bond, and R₅₂ to R₆₁ other than the bond are each independently an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, a group represented by —Si(R₉₀₁)(R₉₀₂)(R₉₀₃), a group represented by —S—(R₉₀₅), a halogen atom, a nitro group, an unsubstituted aryl group having 6 to 50 ring carbon atoms, or an unsubstituted heterocyclic group having 5 to 50 ring atoms;
the heterocyclic groups as R₂₀ to R₂₉ other than the bond, R₃₀ to R₄₁ other than the bond, R₄₂ to R₅₁ other than the bond, and R₅₂ to R₆₁ other than the bond are a heterocyclic group only including at least one atom selected from the group consisting of an oxygen atom, a sulfur atom, and a silicon atom as a hetero atom;
R₉₀₁ to R₉₀₃ and R₉₀₅ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
the heterocyclic group as each of R₉₀₁ to R₉₀₃ and R₉₀₅ is a heterocyclic group only including at least one atom selected from the group consisting of an oxygen atom, a sulfur atom, and a silicon atom as a hetero atom;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different; and
when two or more R₉₀₅ are present, the two or more R₉₀₅ are mutually the same or different.

In the compound according to the exemplary embodiment, the substituent Rx is also preferably each independently a substituted or unsubstituted pyrenyl group and is represented by the formula (Rx-1).

In the compound according to the exemplary embodiment, the groups represented by the formulae (Rx-1) to (Rx-4) are also preferably groups represented by formulae (Rx-1A) to (Rx-4A) below, respectively.

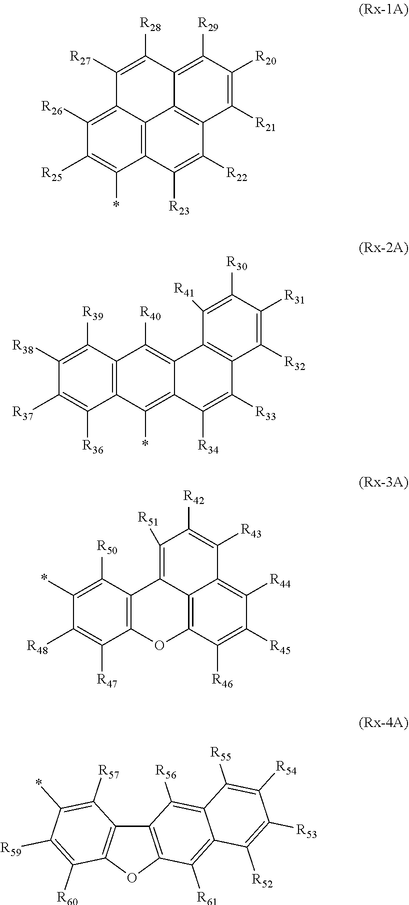

R₂₀ to R₂₉, R₃₀ to R₄₁, R₄₂ to R₅₁, and R₅₂ to R₆₁ in the formulae (Rx-1A) to (Rx-4A) each represent the same as R₂₀ to R₂₉, R₃₀ to R₄₁, R₄₂ to R₅₁, and R₅₂ to R₆₁ in the formulae (Rx-1) to (Rx-4), and * represents a bond of the substituent Rx.

In the compound according to the exemplary embodiment, Rx is also preferably each independently a substituted or unsubstituted pyrenyl group and is represented by the formula (Rx-1A).

In the compound according to the exemplary embodiment, it is also preferable that the substituent Ry is each independently an unsubstituted phenyl group, an unsubstituted naphthyl group, or an unsubstituted dibenzofuranyl group.

In the compound according to the exemplary embodiment, the compound represented by the formula (1) is also preferably a compound represented by a formula (1A) below.

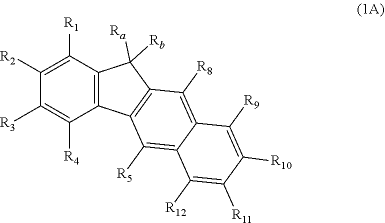

In the formula (1A): Rₐ, R_{b}, R₁ to R₅, and R₈ each represent the same as Rₐ, R_{b}, R₁ to R₅, and R₈ in the formula (1); and R₉ to R₁₂ each independently represent the same as R₉ to R₁₂ in the formula (10).

In the compound according to the exemplary embodiment, it is also preferable that R₂ in the formula (1A) is the substituent Rx, and R₄ or R₅ is the substituent Ry.

In the compound according to the exemplary embodiment, Rₐ and R_{b} in the formula (1A) are also preferably each independently a substituted or unsubstituted alkyl group having 1 to 4 carbon atoms.

In the compound according to the exemplary embodiment, R₁ to R₅ and R₈ to R₁₂ being neither the substituent Rx nor the substituent Ry in the formula (1A) are also preferably each a hydrogen atom.

In the compound according to the exemplary embodiment, the compound represented by the formula (1A) is also preferably a compound represented by a formula (1A-A), (1A-B), (1A-C), or (1A-D) below.

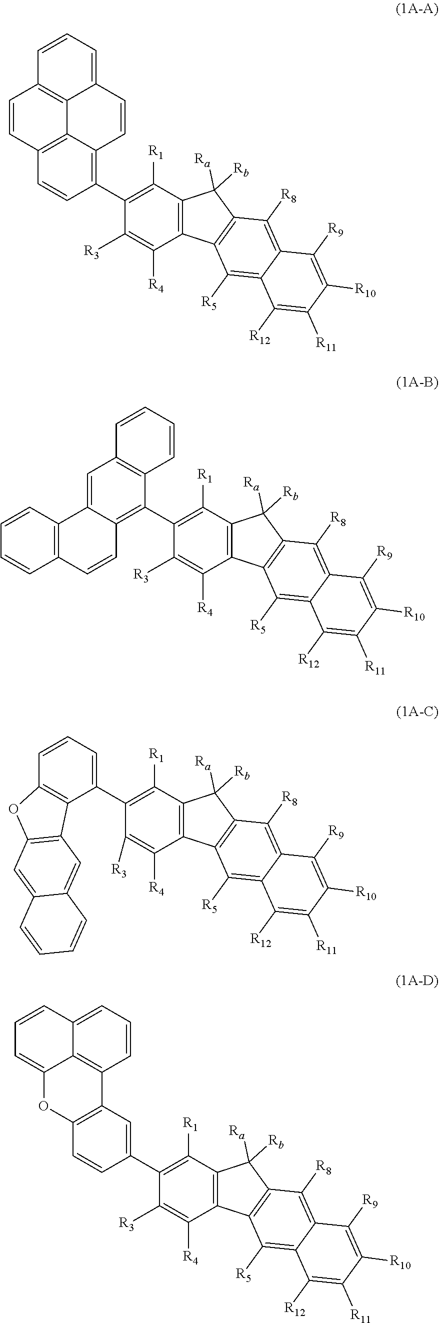

In the formulae (1A-A), (1A-B), (1A-C), and (1A-D): R₄ or R₅ is each independently the substituent Ry; Rₐ, R_{b}, R₁, R₃, and R₈ represent each independently the same as Rₐ, R_{b}, R₁, R₃, and R₈ in the formula (1); and R₉ to R₁₂ each independently represent the same as R₉ to R₁₂ in the formula (10).

In the compound according to the exemplary embodiment, the compound represented by the formula (1) is also preferably a compound represented by a formula (1B) below.

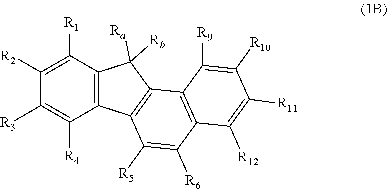

In the formula (1B): Rₐ, R_{b} and R₁ to R₆ each independently represent the same as Rₐ, R_{b} and R₁ to R₆ in the formula (1); and R₉ to R₁₂ each independently represent the same as R₉ to R₁₂ in the formula (10).

In the compound according to the exemplary embodiment, it is also preferable that R₃ in the formula (1B) is the substituent Rx and R₁₂ is the substituent Ry.

In the compound according to the exemplary embodiment, Rₐ and R_{b} in the formula (1B) are also preferably each independently a substituted or unsubstituted alkyl group having 1 to 4 carbon atoms.

In the compound according to the exemplary embodiment, R₁ to R₆ and R₉ to R₁₂ being neither the substituent Rx nor the substituent Ry in the formula (1B) are also preferably each a hydrogen atom.

In the compound according to the exemplary embodiment, the compound represented by the formula (1B) is also preferably a compound represented by a formula (1B-A), (1B-B), (1B-C), or (1B-D) below.

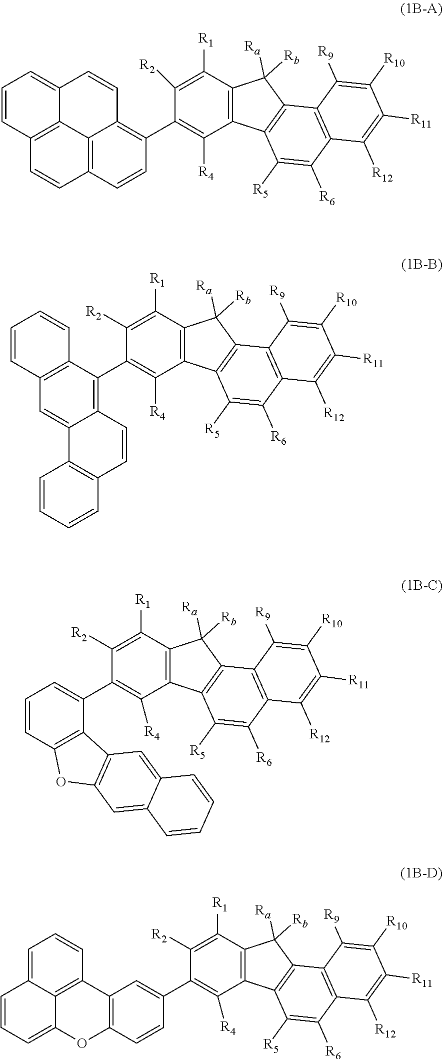

In the formulae (1B-A), (1B-B), (1B-C), and (1B-D): R₁₂ is the substituent Ry; Rₐ, R_{b}, R₁, R₂, and R₄ to R₆ each independently represent the same as Rₐ, R_{b}, R₁, R₂, and R₄ to R₆ in the formula (1); and R₉ to R₁₁ each independently represent the same as R₉ to R₁₁ in the formula (10).

In the compound according to the exemplary embodiment, the compound represented by the formula (1) is also preferably a compound represented by a formula (1C) below.

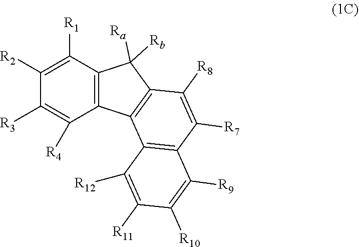

In the formula (1C): Rₐ, R_{b}, R₁ to R₄, R₇, and R₈ each represent the same as Rₐ, R_{b}, R₁ to R₄, R₇, and R₈ in the formula (1); and R₉ to R₁₂ each independently represent the same as R₉ to R₁₂ in the formula (10).

In the compound according to the exemplary embodiment, it is also preferable that R₂ in the formula (1C) is the substituent Rx, and R₉ is the substituent Ry.

In the compound according to the exemplary embodiment, Rₐ and R_{b} in the formula (1C) are also preferably each independently a substituted or unsubstituted alkyl group having 1 to 4 carbon atoms.

In the compound according to the exemplary embodiment, R₁ to R₄ and R₇ to R₁₂ being neither the substituent Rx nor the substituent Ry in the formula (1C) are also preferably each a hydrogen atom.

In the compound according to the exemplary embodiment, the compound represented by the formula (1C) is also preferably a compound represented by a formula (1C-A), (1C-B), (1C-C), or (1C-D) below.

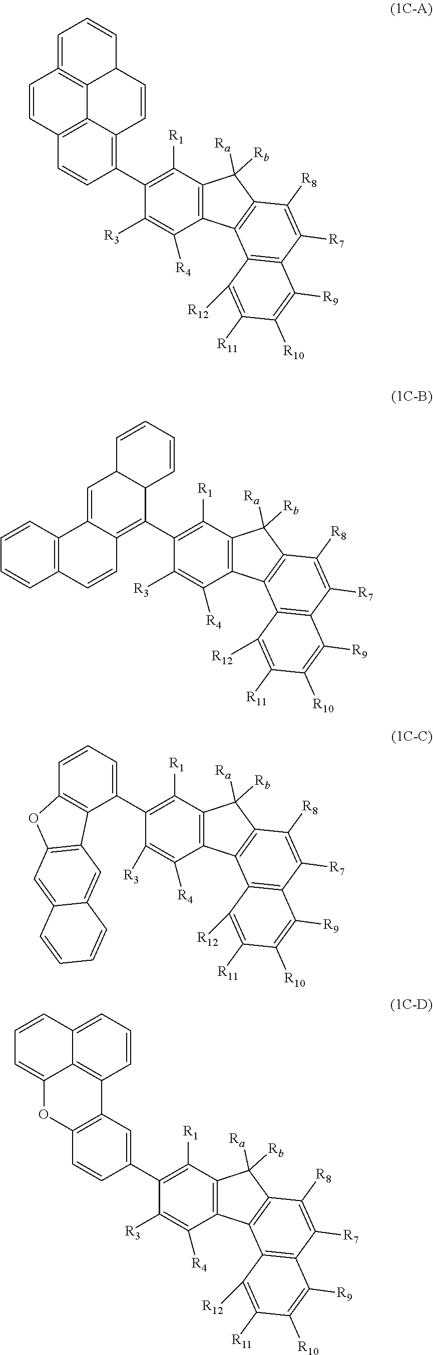

In the formulae (1C-A), (1C-B), (1C-C), and (1C-D): R₉ is the substituent Ry; Rₐ, R_{b}, R₁, R₃, R₄, R₇ and R₈ each independently represent the same as Rₐ, R_{b}, R₁, R₃, R₄, R₇ and R₈ in the formula (1); and R₁₀ to R₁₂ each independently represent the same as R₁₀ to R₁₂ in the formula (10).

In the compound according to the exemplary embodiment, the substituent for the substituted or unsubstituted group is preferably a halogen atom, an unsubstituted alkyl group having 1 to 25 carbon atoms, an unsubstituted aryl group having 6 to 25 ring carbon atoms, or an unsubstituted heterocyclic group having 5 to 25 ring atoms.

In the compound according to the exemplary embodiment, the substituent for the substituted or unsubstituted group is preferably an unsubstituted alkyl group having 1 to 10 carbon atoms, an unsubstituted aryl group having 6 to 12 ring carbon atoms, or an unsubstituted heterocyclic group having 5 to 12 ring atoms.

In the compound according to the exemplary arrangement, the groups specified to be “substituted or unsubstituted” are each preferably an “unsubstituted” group.

### Producing Method of Compound in the Exemplary Embodiment

The compound according to the exemplary embodiment can be produced in accordance with a synthesis method described later in Examples. Further, the compound according to the exemplary embodiment can be produced by application of known substitution reactions and materials tailored for the target compound, in accordance with the synthesis method described later in Examples.

### Specific Examples of Compound in the Exemplary Embodiment

Specific examples of the compound in the exemplary embodiment include compounds below. However, the invention is by no means limited to the specific examples.

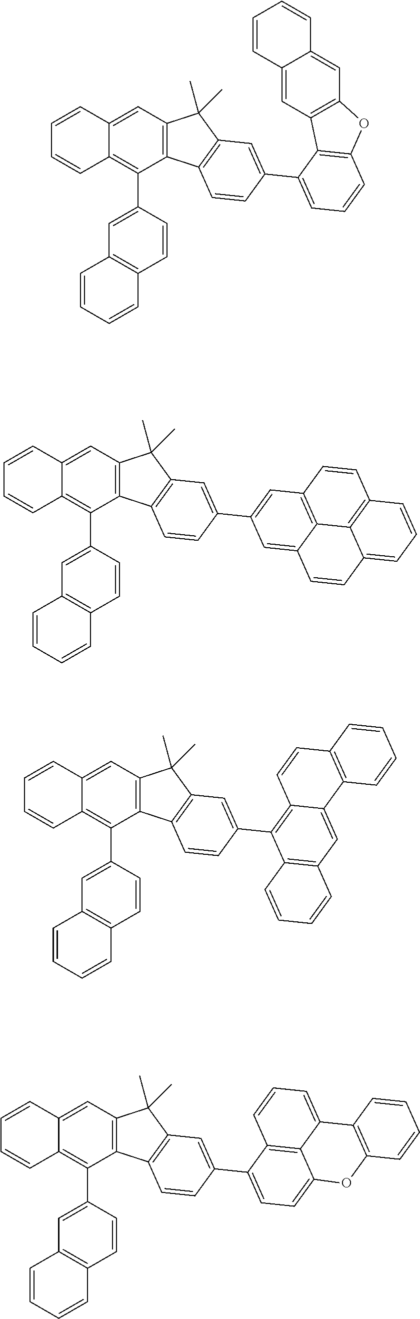

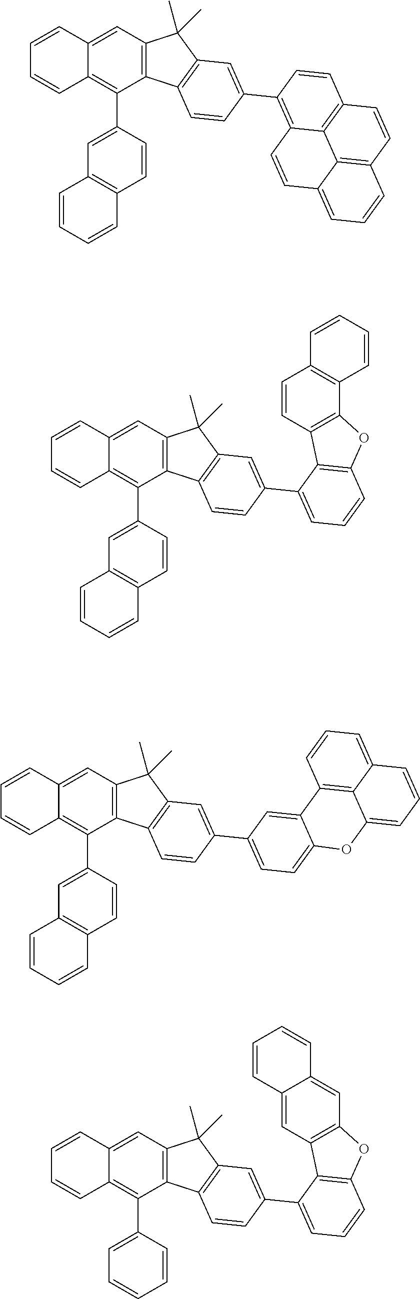

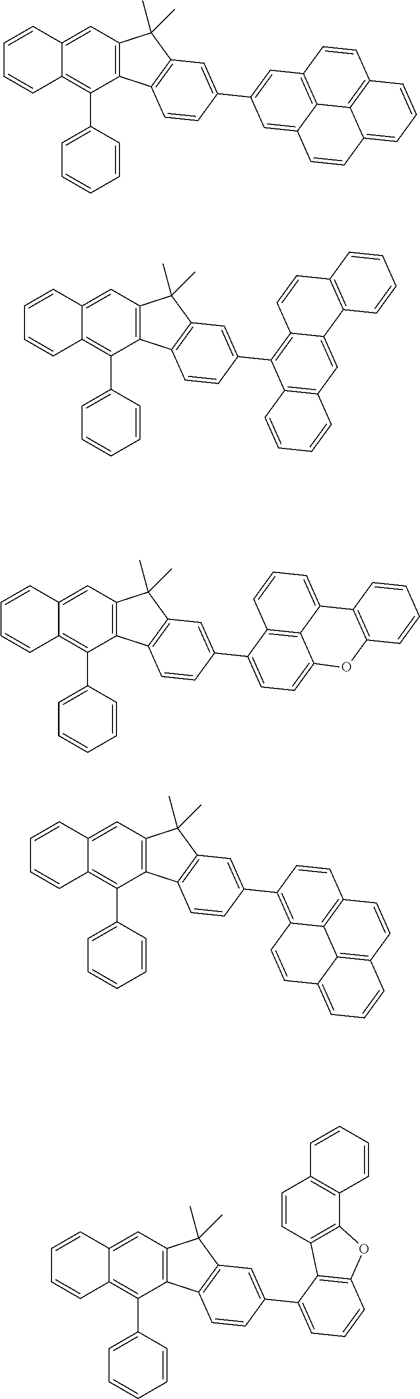

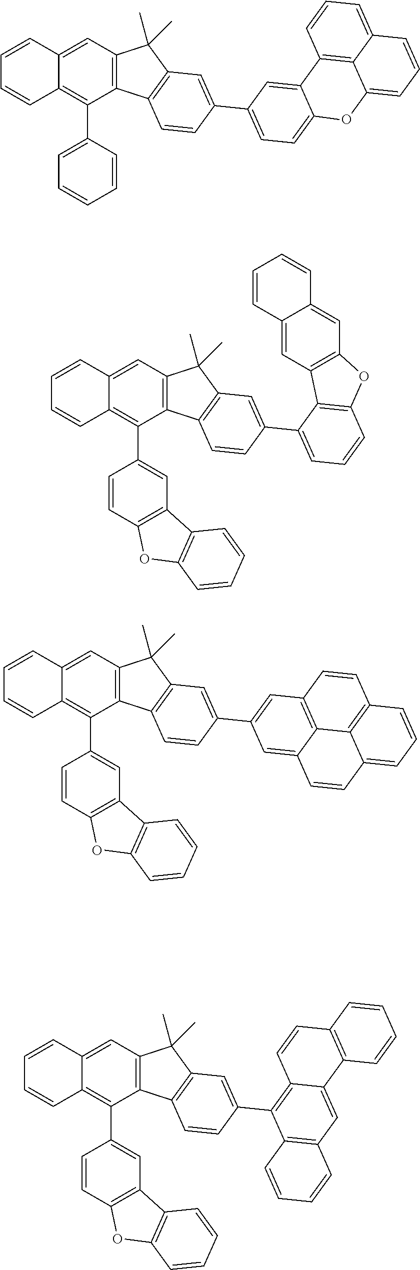 [SMILES: CC1(C)c2cc(-c3ccc4c(c3)-c3cccc5cccc(c35)O4)ccc2-c2c1cc1ccccc1c2-c1ccccc1 ; CC1(C)c2cc(-c3cccc4oc5cc6ccccc6cc5c34)ccc2-c2c1cc1ccccc1c2-c1ccc2oc3ccccc3c2c1 ; CC1(C)c2cc(-c3cc4ccc5cccc6ccc(c3)c4c56)ccc2-c2c1cc1ccccc1c2-c1ccc2oc3ccccc3c2c1 ; CC1(C)c2cc(-c3c4ccccc4cc4c3ccc3ccccc34)ccc2-c2c1cc1ccccc1c2-c1ccc2oc3ccccc3c2c1]

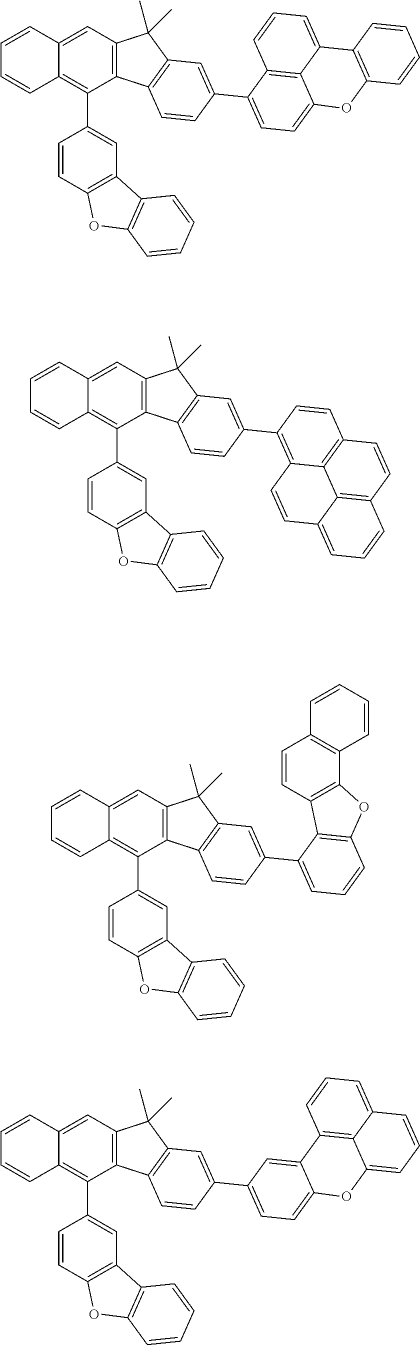 [SMILES: CC1(C)c2cc(-c3ccc4c5c(cccc35)-c3ccccc3O4)ccc2-c2c1cc1ccccc1c2-c1ccc2oc3ccccc3c2c1 ; CC1(C)c2cc(-c3ccc4ccc5cccc6ccc3c4c56)ccc2-c2c1cc1ccccc1c2-c1ccc2oc3ccccc3c2c1 ; CC1(C)c2cc(-c3cccc4oc5c6ccccc6ccc5c34)ccc2-c2c1cc1ccccc1c2-c1ccc2oc3ccccc3c2c1 ; CC1(C)c2cc(-c3ccc4c(c3)-c3cccc5cccc(c35)O4)ccc2-c2c1cc1ccccc1c2-c1ccc2oc3ccccc3c2c1]

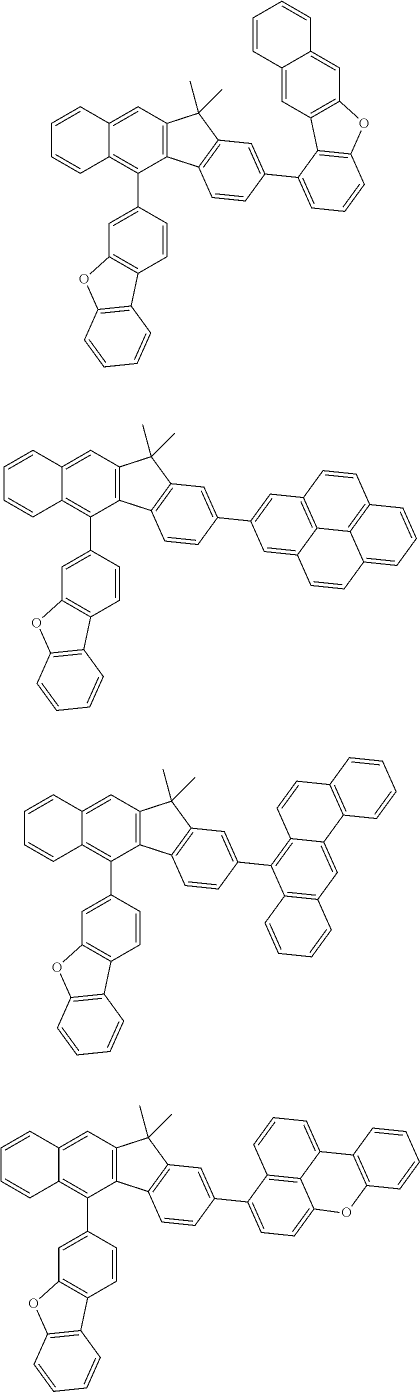 [SMILES: CC1(C)c2cc(-c3cccc4oc5cc6ccccc6cc5c34)ccc2-c2c1cc1ccccc1c2-c1ccc2c(c1)oc1ccccc12 ; CC1(C)c2cc(-c3cc4ccc5cccc6ccc(c3)c4c56)ccc2-c2c1cc1ccccc1c2-c1ccc2c(c1)oc1ccccc12 ; CC1(C)c2cc(-c3c4ccccc4cc4c3ccc3ccccc34)ccc2-c2c1cc1ccccc1c2-c1ccc2c(c1)oc1ccccc12 ; CC1(C)c2cc(-c3ccc4c5c(cccc35)-c3ccccc3O4)ccc2-c2c1cc1ccccc1c2-c1ccc2c(c1)oc1ccccc12]

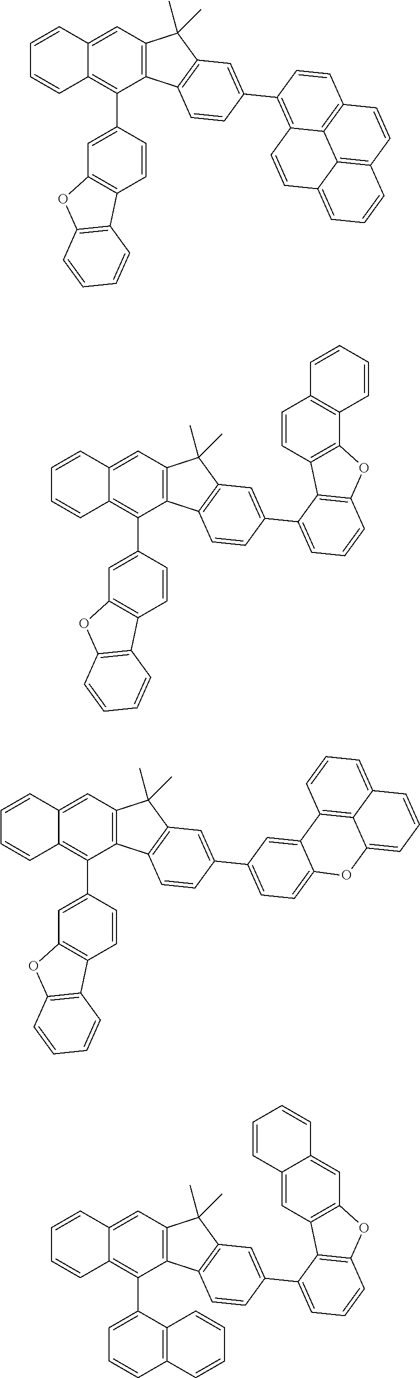 [SMILES: CC1(C)c2cc(-c3ccc4ccc5cccc6ccc3c4c56)ccc2-c2c1cc1ccccc1c2-c1ccc2c(c1)oc1ccccc12 ; CC1(C)c2cc(-c3cccc4oc5c6ccccc6ccc5c34)ccc2-c2c1cc1ccccc1c2-c1ccc2c(c1)oc1ccccc12 ; CC1(C)c2cc(-c3ccc4c(c3)-c3cccc5cccc(c35)O4)ccc2-c2c1cc1ccccc1c2-c1ccc2c(c1)oc1ccccc12 ; CC1(C)c2cc(-c3cccc4oc5cc6ccccc6cc5c34)ccc2-c2c1cc1ccccc1c2-c1cccc2ccccc12]

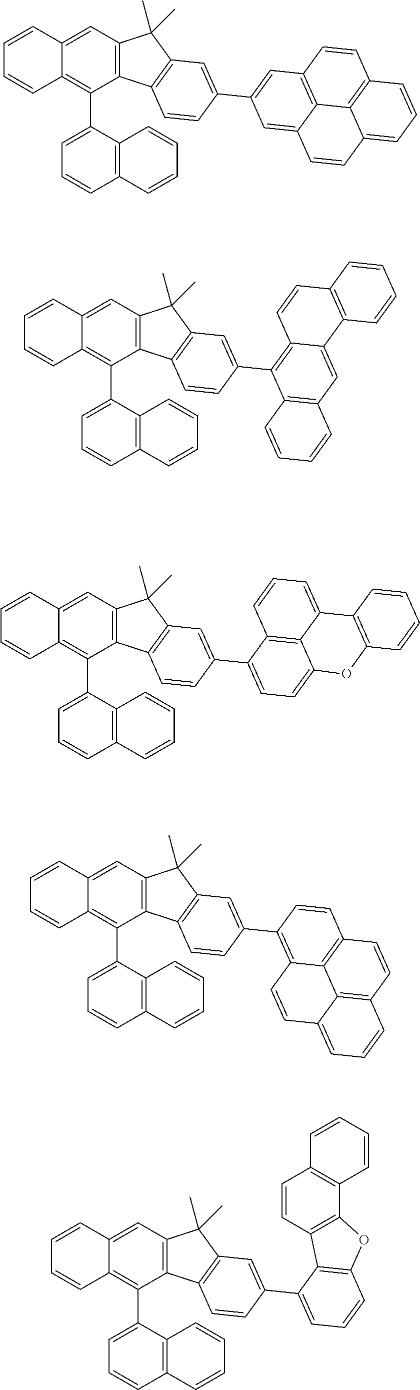

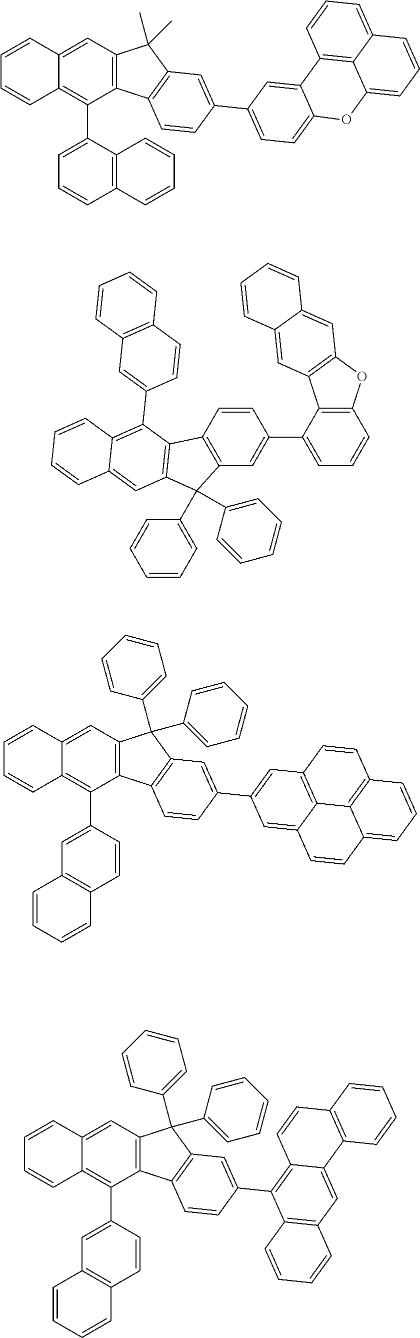

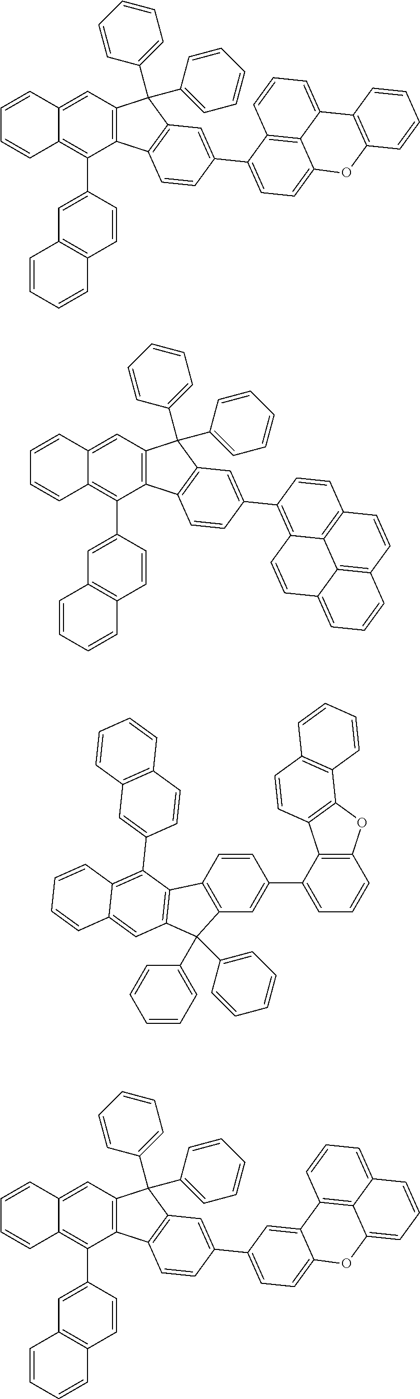

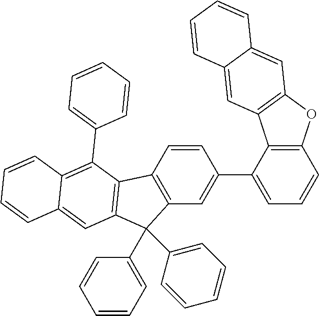 [SMILES: c1ccc(-c2c3c(cc4ccccc24)C(c2ccccc2)(c2ccccc2)c2cc(-c4cccc5oc6cc7ccccc7cc6c45)ccc2-3)cc1]

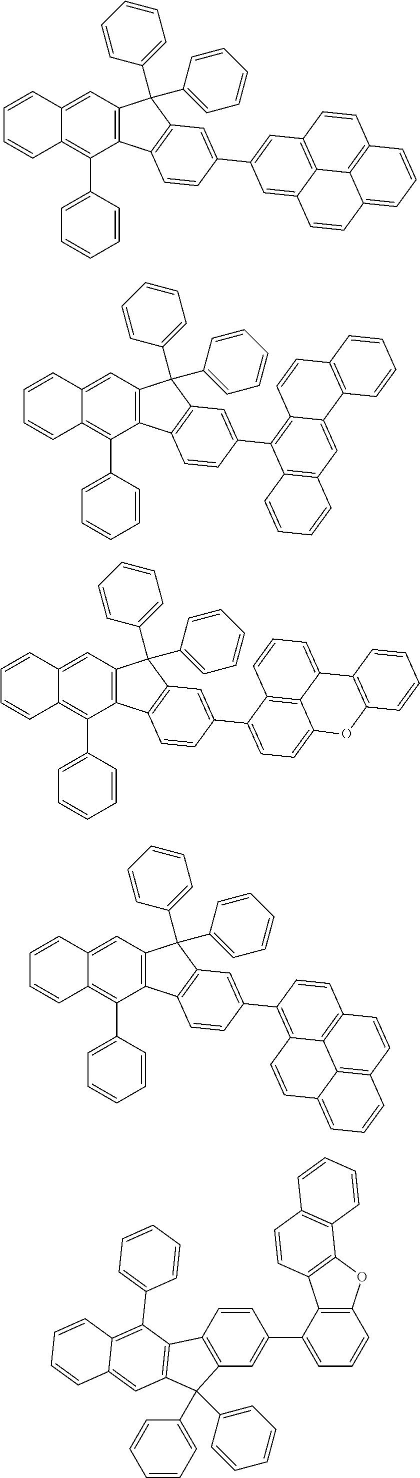

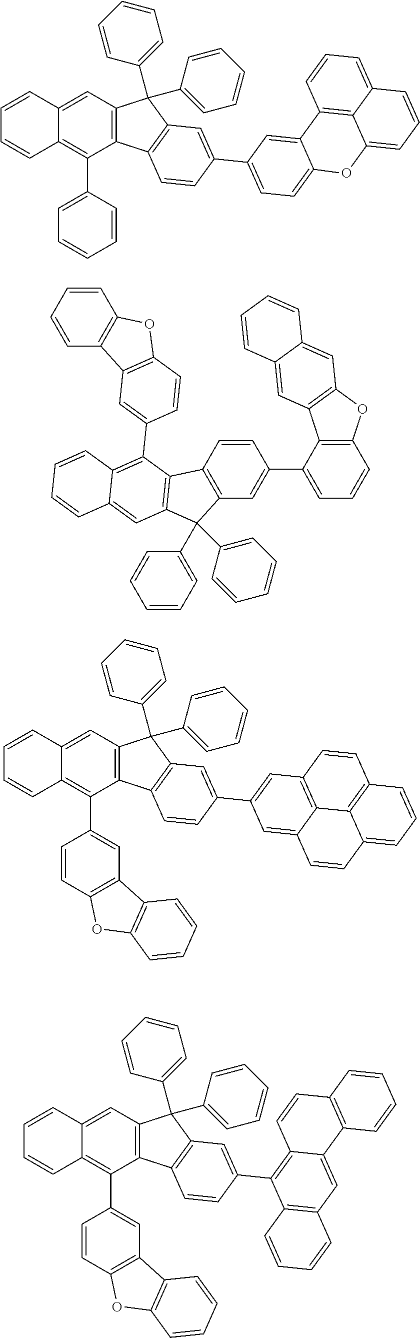

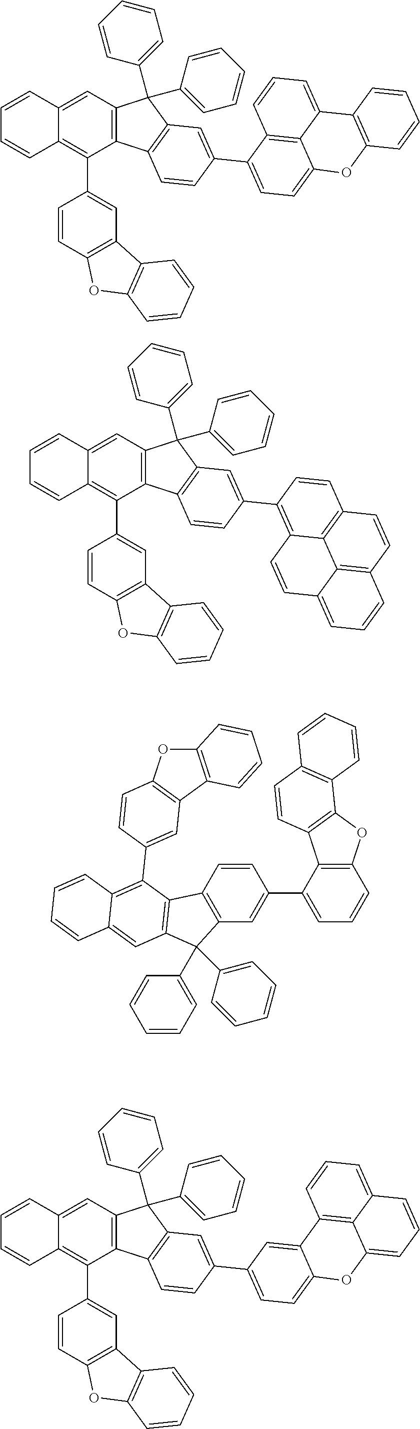

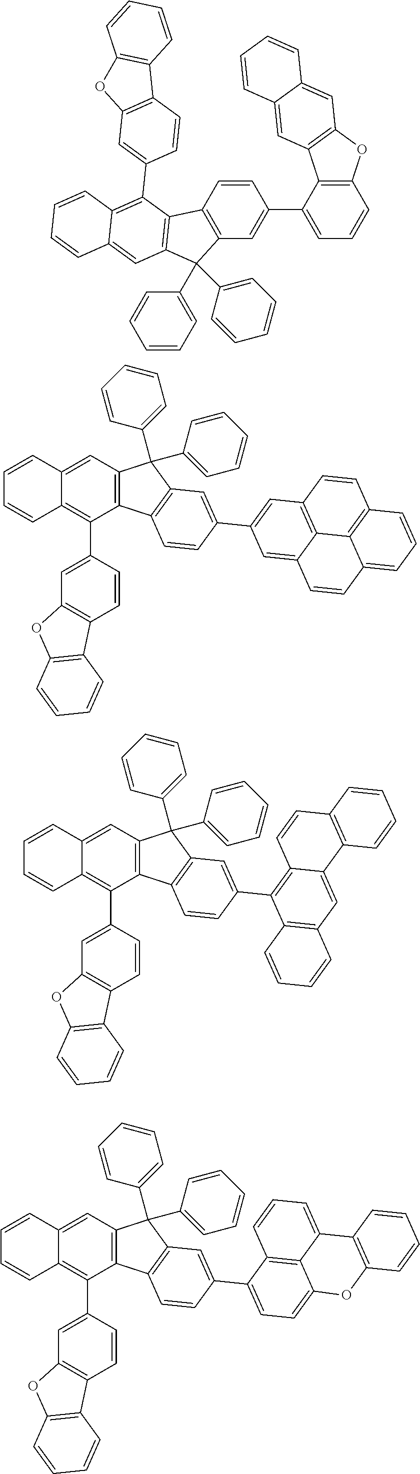

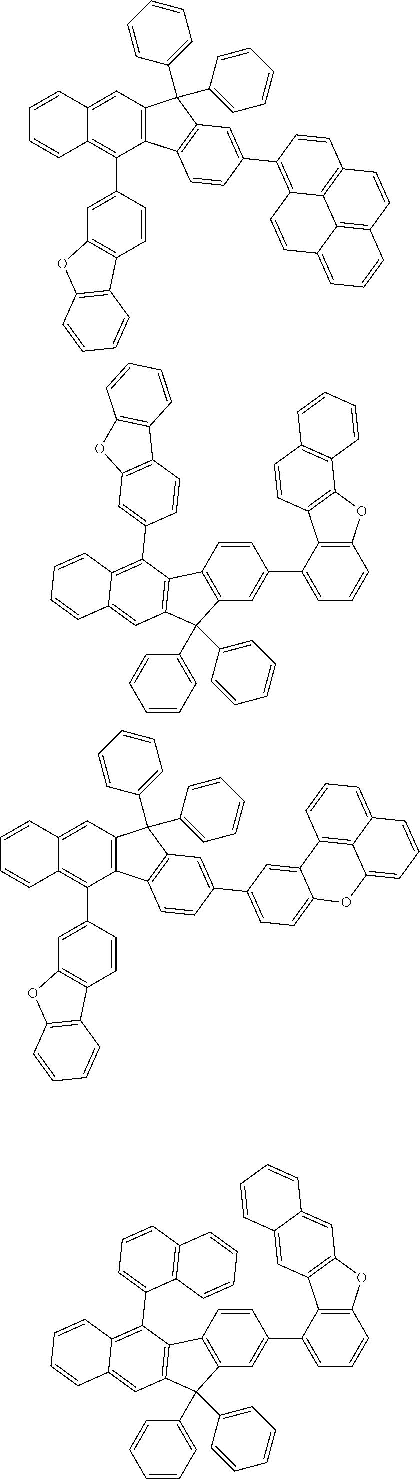

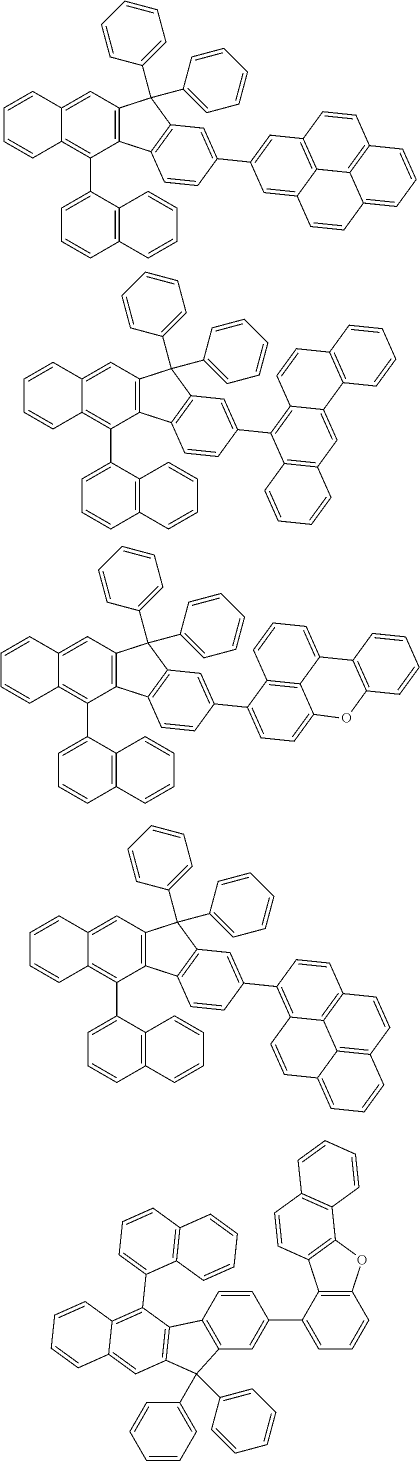

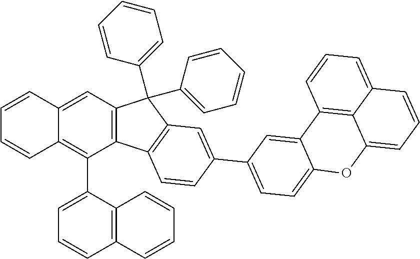 [SMILES: c1ccc(C2(c3ccccc3)c3cc(-c4ccc5c(c4)-c4cccc6cccc(c46)O5)ccc3-c3c2cc2ccccc2c3-c2cccc3ccccc23)cc1]

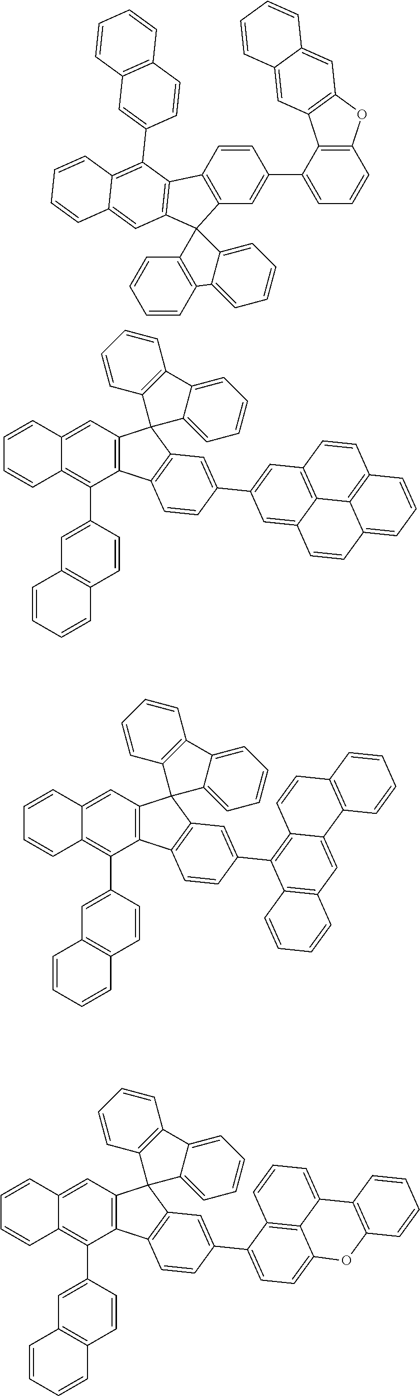

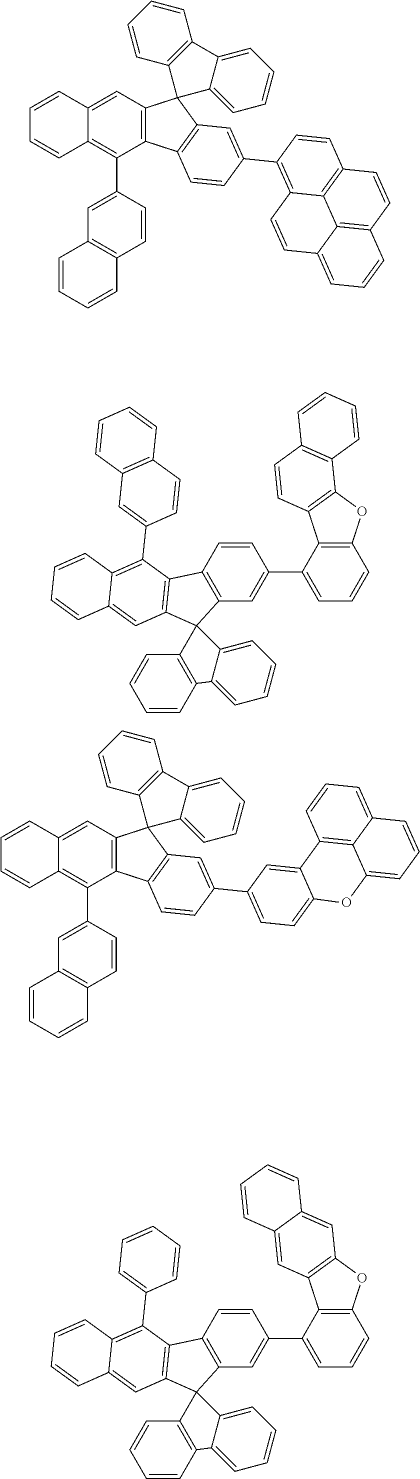

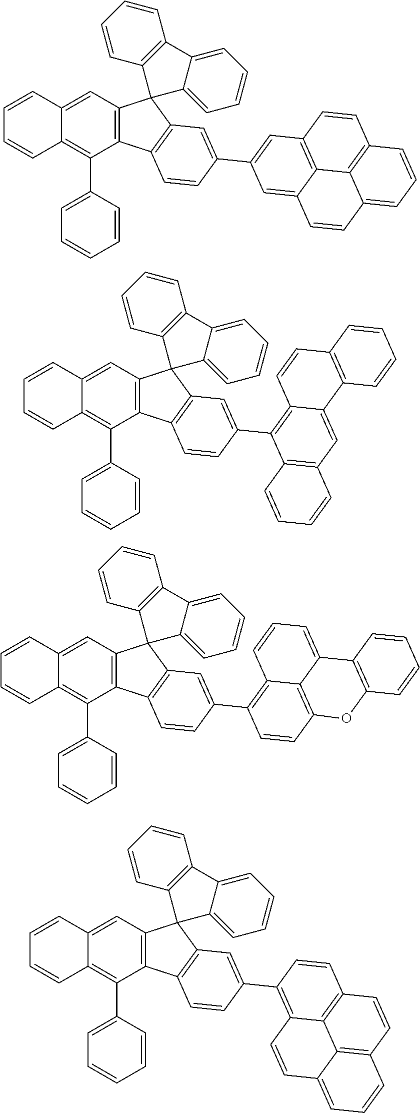

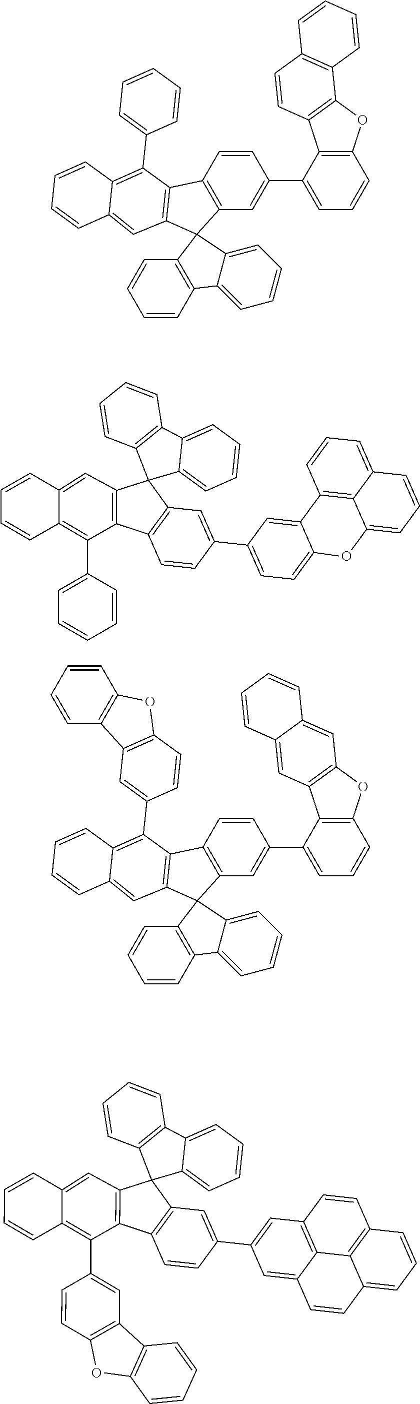

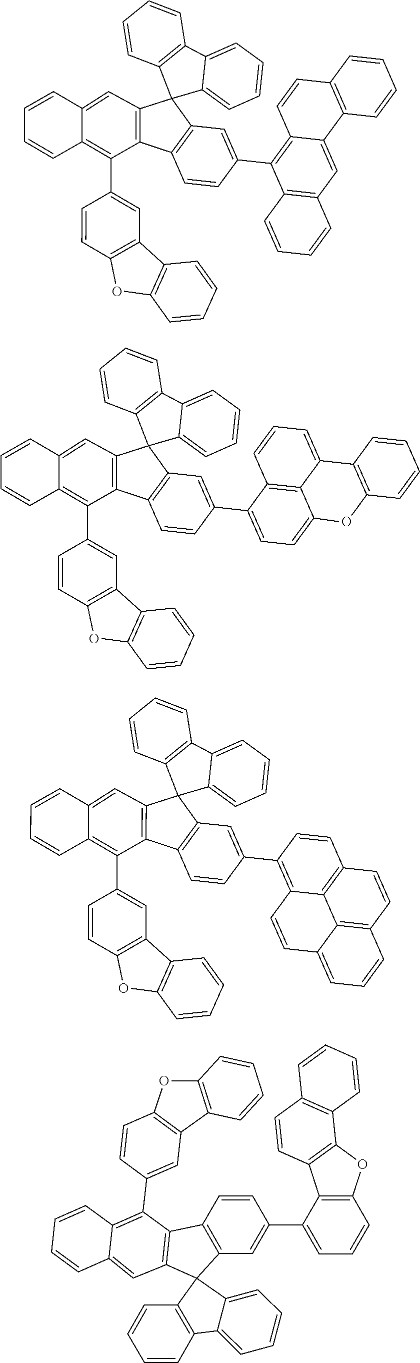

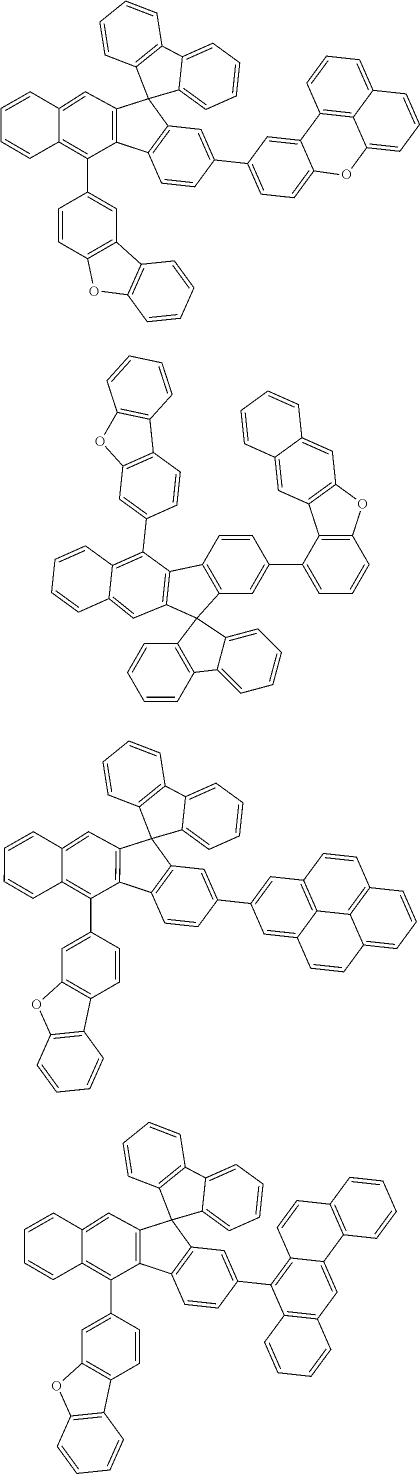

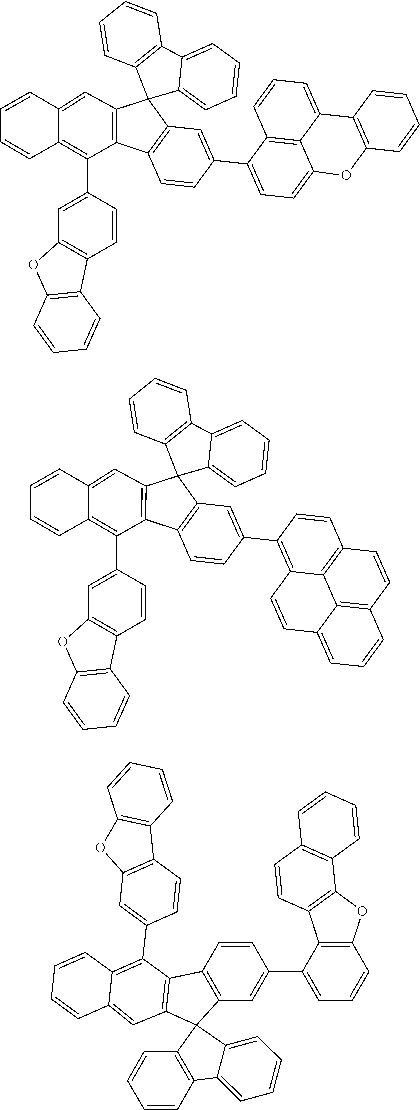

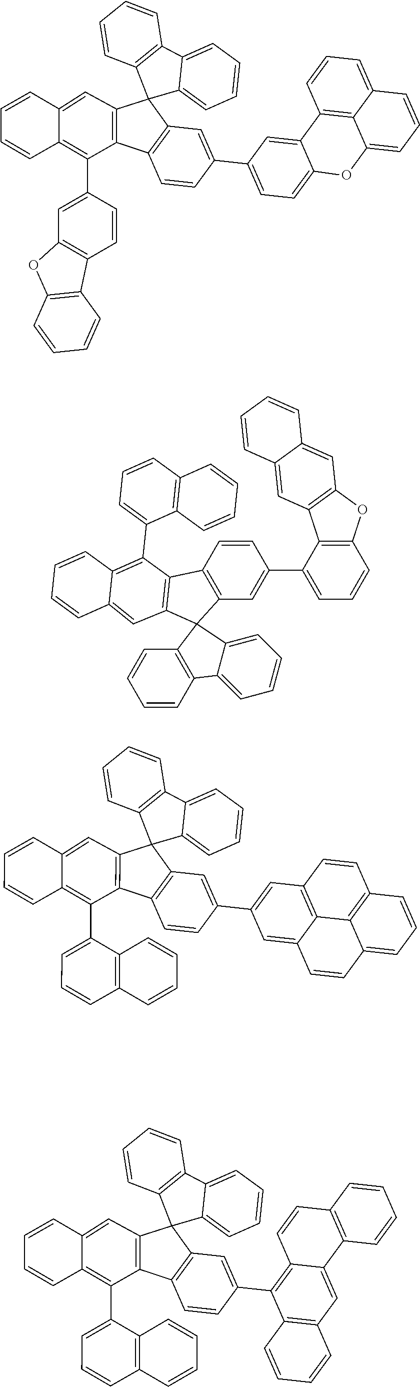

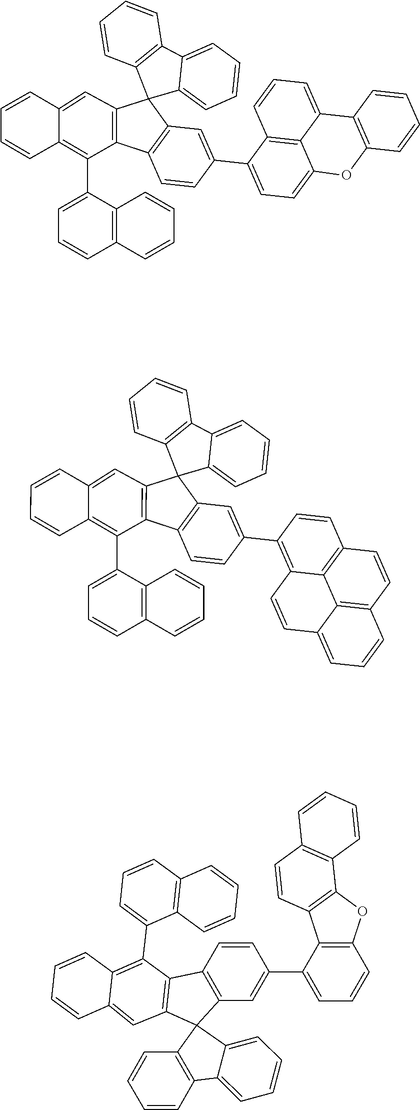

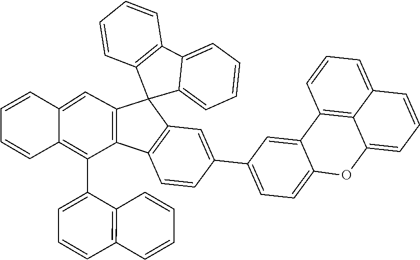 [SMILES: c1ccc2c(c1)-c1ccccc1C21c2cc(-c3ccc4c(c3)-c3cccc5cccc(c35)O4)ccc2-c2c1cc1ccccc1c2-c1cccc2ccccc12]

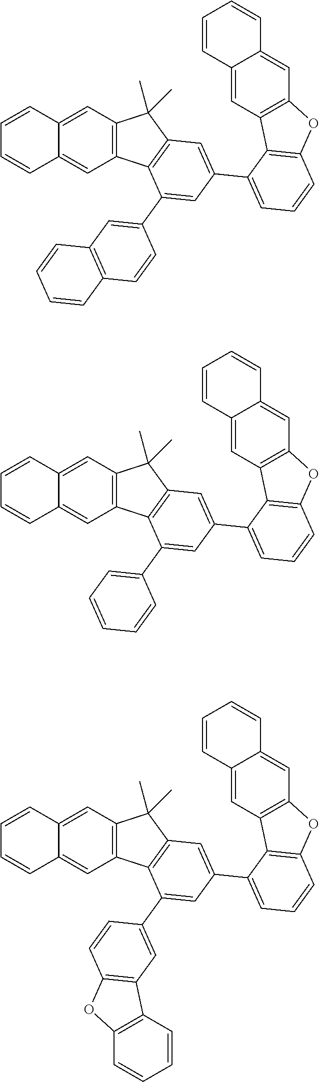 [SMILES: CC1(C)c2cc3ccccc3cc2-c2c(-c3ccc4ccccc4c3)cc(-c3cccc4oc5cc6ccccc6cc5c34)cc21 ; CC1(C)c2cc3ccccc3cc2-c2c(-c3ccccc3)cc(-c3cccc4oc5cc6ccccc6cc5c34)cc21 ; CC1(C)c2cc3ccccc3cc2-c2c(-c3ccc4oc5ccccc5c4c3)cc(-c3cccc4oc5cc6ccccc6cc5c34)cc21]

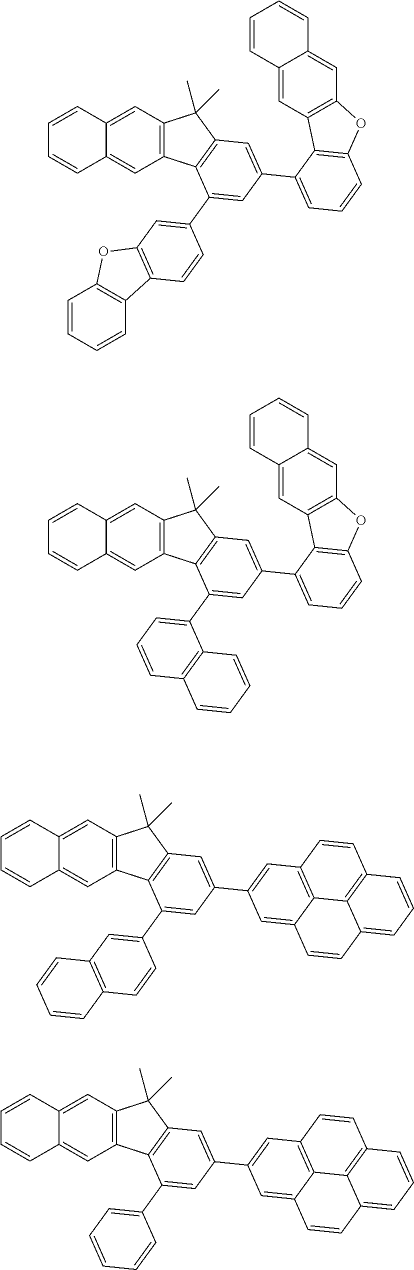

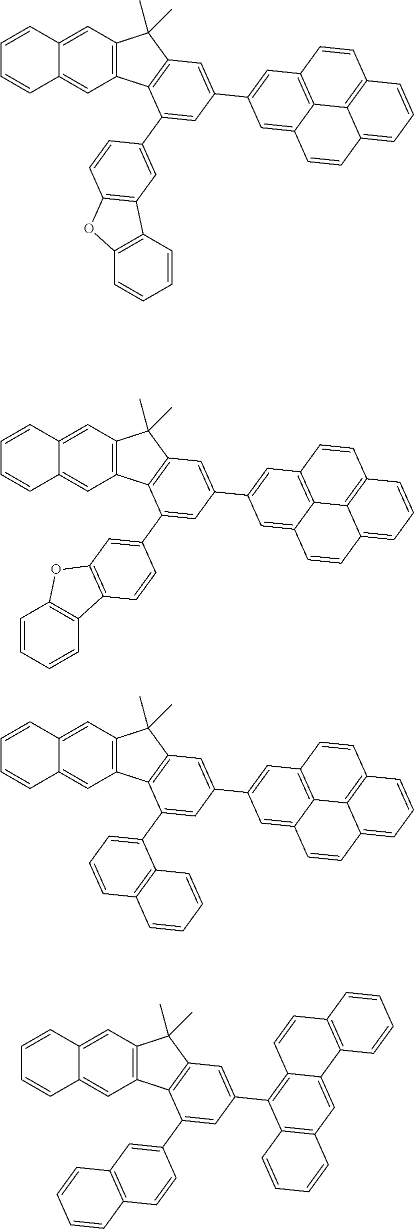

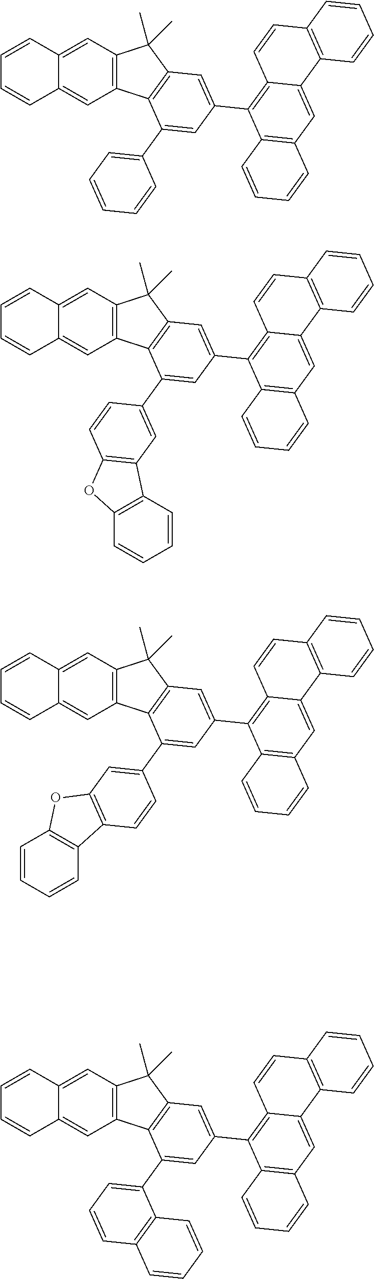

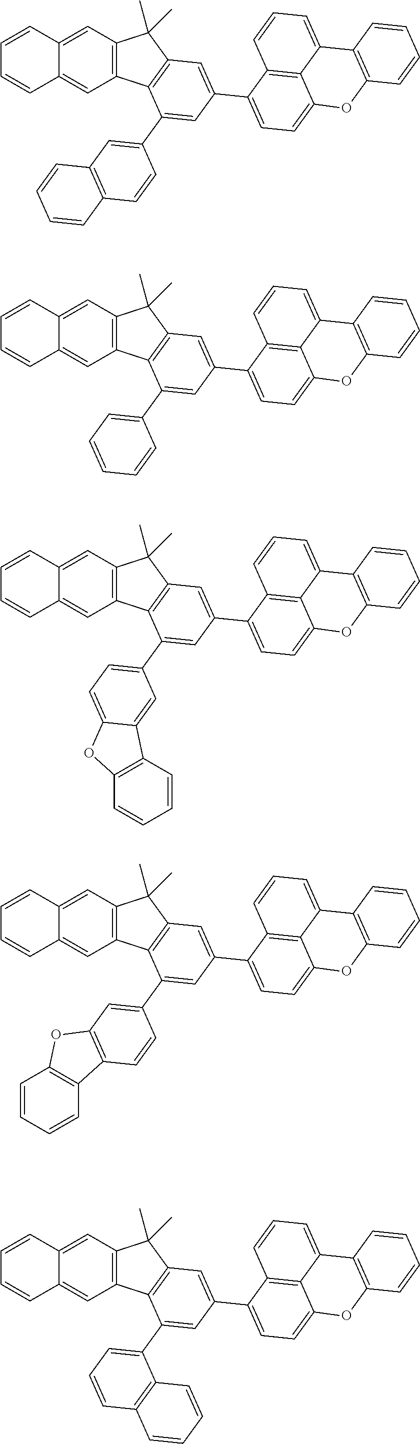

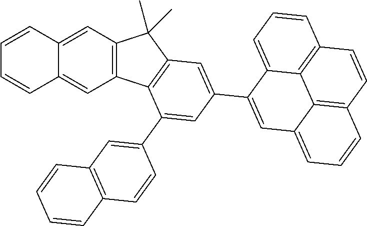 [SMILES: CC1(C)c2cc3ccccc3cc2-c2c(-c3ccc4ccccc4c3)cc(-c3cc4cccc5ccc6cccc3c6c54)cc21]

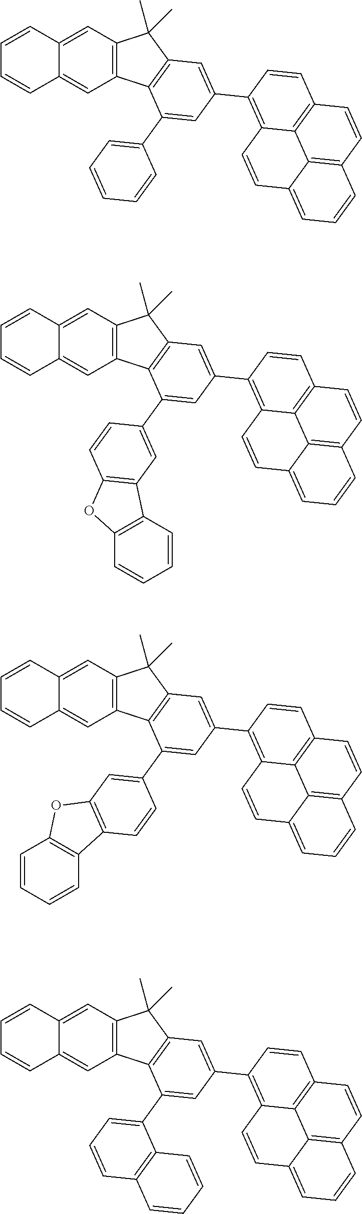

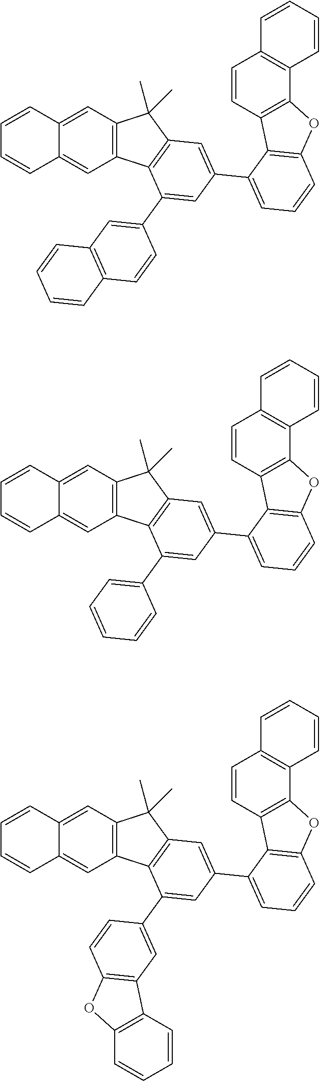 [SMILES: CC1(C)c2cc3ccccc3cc2-c2c(-c3ccc4ccccc4c3)cc(-c3cccc4oc5c6ccccc6ccc5c34)cc21 ; CC1(C)c2cc3ccccc3cc2-c2c(-c3ccccc3)cc(-c3cccc4oc5c6ccccc6ccc5c34)cc21 ; CC1(C)c2cc3ccccc3cc2-c2c(-c3ccc4oc5ccccc5c4c3)cc(-c3cccc4oc5c6ccccc6ccc5c34)cc21]

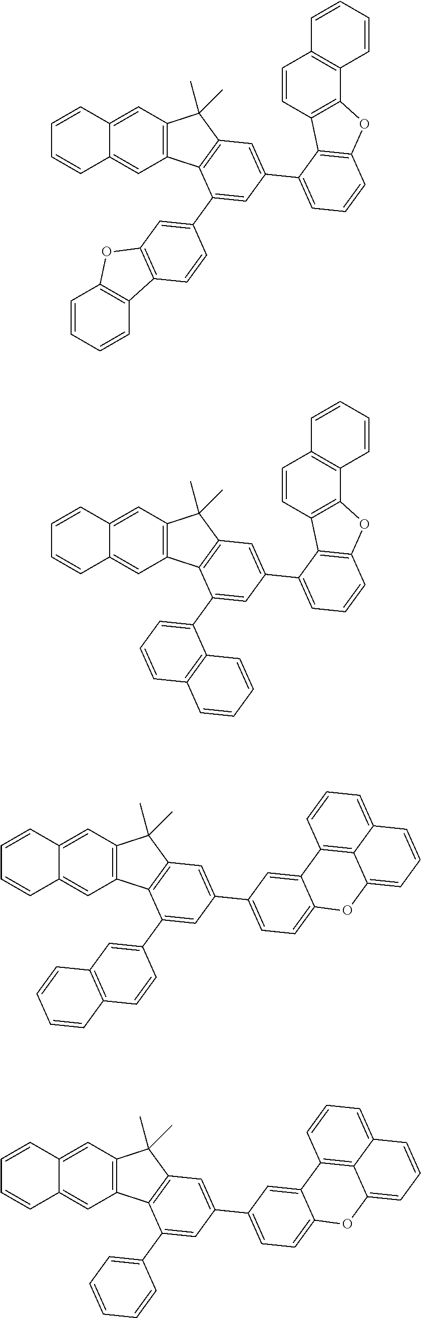 [SMILES: CC1(C)c2cc3ccccc3cc2-c2c(-c3ccc4c(c3)oc3ccccc34)cc(-c3cccc4oc5c6ccccc6ccc5c34)cc21 ; CC1(C)c2cc3ccccc3cc2-c2c(-c3cccc4ccccc34)cc(-c3cccc4oc5c6ccccc6ccc5c34)cc21 ; CC1(C)c2cc3ccccc3cc2-c2c(-c3ccc4ccccc4c3)cc(-c3ccc4c(c3)-c3cccc5cccc(c35)O4)cc21 ; CC1(C)c2cc3ccccc3cc2-c2c(-c3ccccc3)cc(-c3ccc4c(c3)-c3cccc5cccc(c35)O4)cc21]

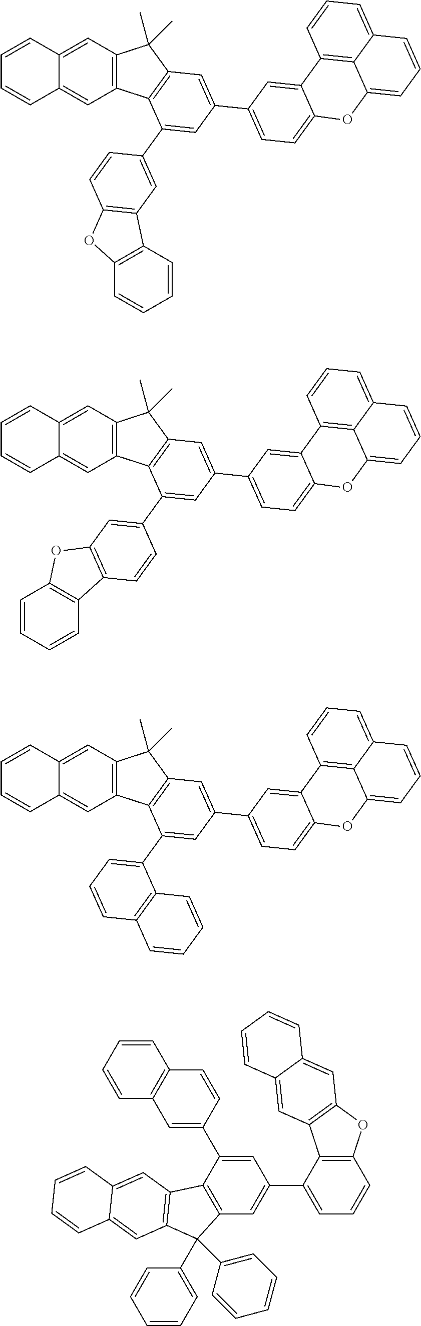 [SMILES: CC1(C)c2cc3ccccc3cc2-c2c(-c3ccc4oc5ccccc5c4c3)cc(-c3ccc4c(c3)-c3cccc5cccc(c35)O4)cc21 ; CC1(C)c2cc3ccccc3cc2-c2c(-c3ccc4c(c3)oc3ccccc34)cc(-c3ccc4c(c3)-c3cccc5cccc(c35)O4)cc21 ; CC1(C)c2cc3ccccc3cc2-c2c(-c3cccc4ccccc34)cc(-c3ccc4c(c3)-c3cccc5cccc(c35)O4)cc21 ; c1ccc(C2(c3ccccc3)c3cc4ccccc4cc3-c3c(-c4ccc5ccccc5c4)cc(-c4cccc5oc6cc7ccccc7cc6c45)cc32)cc1]

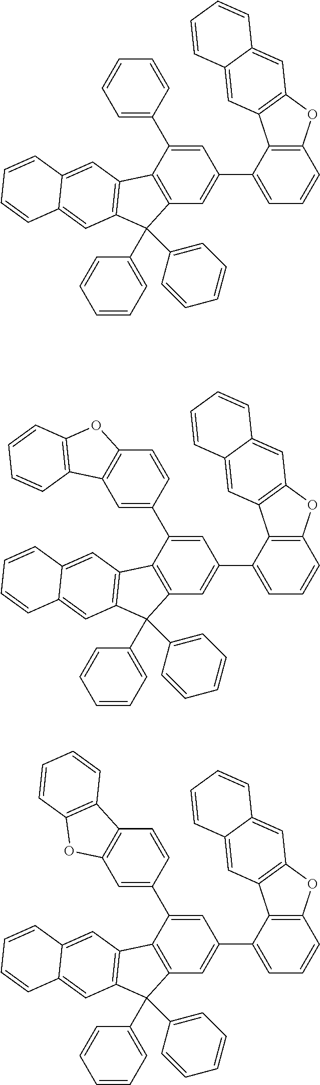 [SMILES: c1ccc(-c2cc(-c3cccc4oc5cc6ccccc6cc5c34)cc3c2-c2cc4ccccc4cc2C3(c2ccccc2)c2ccccc2)cc1 ; c1ccc(C2(c3ccccc3)c3cc4ccccc4cc3-c3c(-c4ccc5oc6ccccc6c5c4)cc(-c4cccc5oc6cc7ccccc7cc6c45)cc32)cc1 ; c1ccc(C2(c3ccccc3)c3cc4ccccc4cc3-c3c(-c4ccc5c(c4)oc4ccccc45)cc(-c4cccc5oc6cc7ccccc7cc6c45)cc32)cc1]

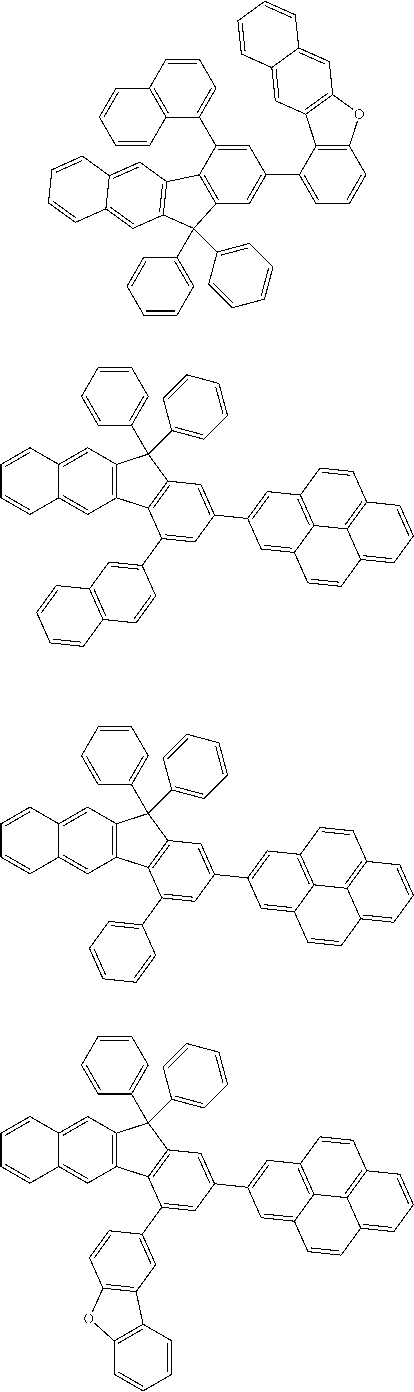

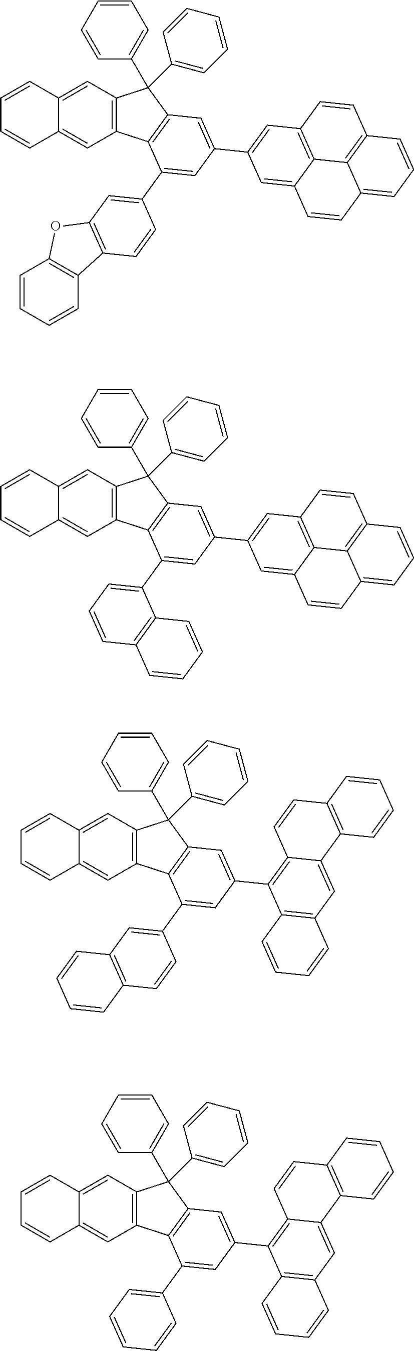

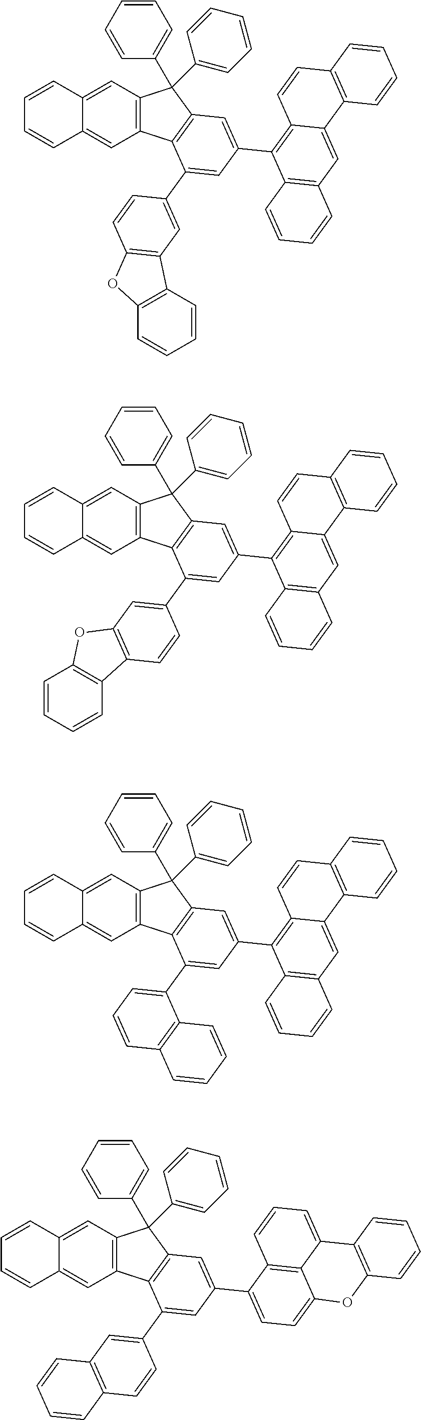

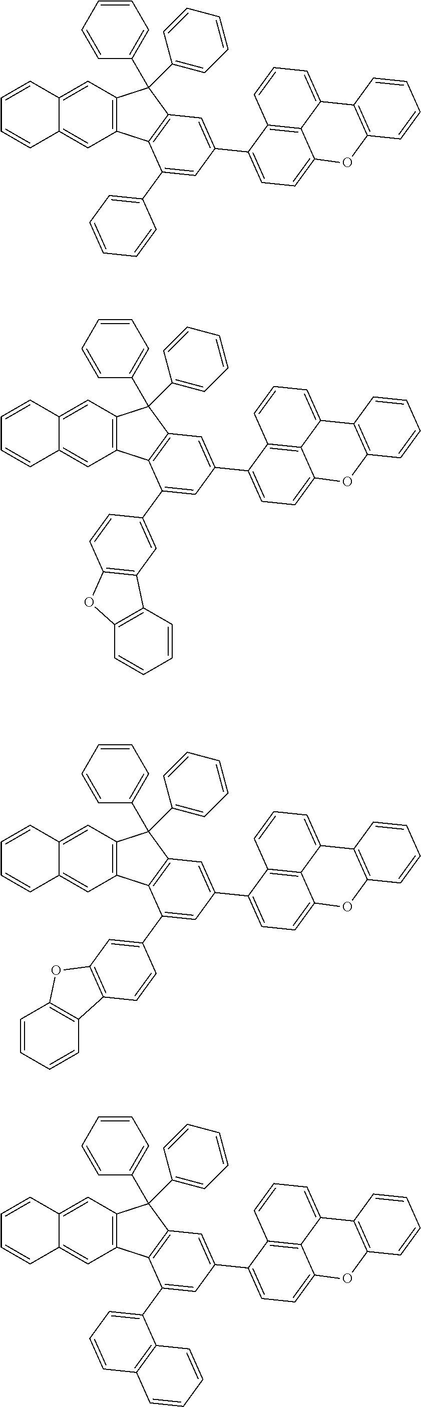 [SMILES: c1ccc(-c2cc(-c3ccc4c5c(cccc35)-c3ccccc3O4)cc3c2-c2cc4ccccc4cc2C3(c2ccccc2)c2ccccc2)cc1 ; c1ccc(C2(c3ccccc3)c3cc4ccccc4cc3-c3c(-c4ccc5oc6ccccc6c5c4)cc(-c4ccc5c6c(cccc46)-c4ccccc4O5)cc32)cc1 ; c1ccc(C2(c3ccccc3)c3cc4ccccc4cc3-c3c(-c4ccc5c(c4)oc4ccccc45)cc(-c4ccc5c6c(cccc46)-c4ccccc4O5)cc32)cc1 ; c1ccc(C2(c3ccccc3)c3cc4ccccc4cc3-c3c(-c4cccc5ccccc45)cc(-c4ccc5c6c(cccc46)-c4ccccc4O5)cc32)cc1]

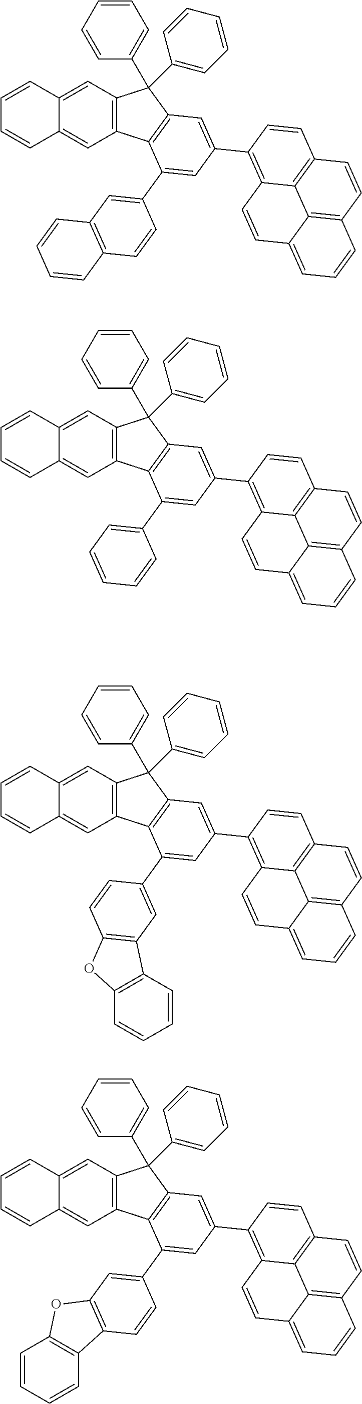

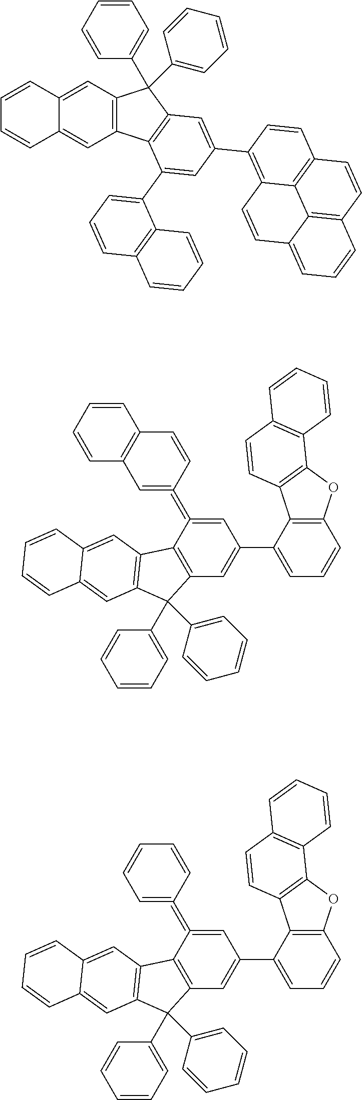

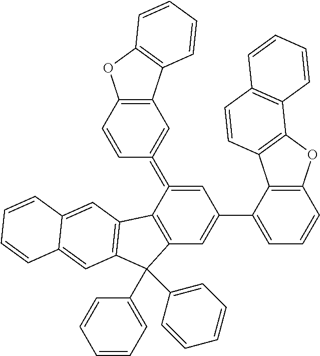

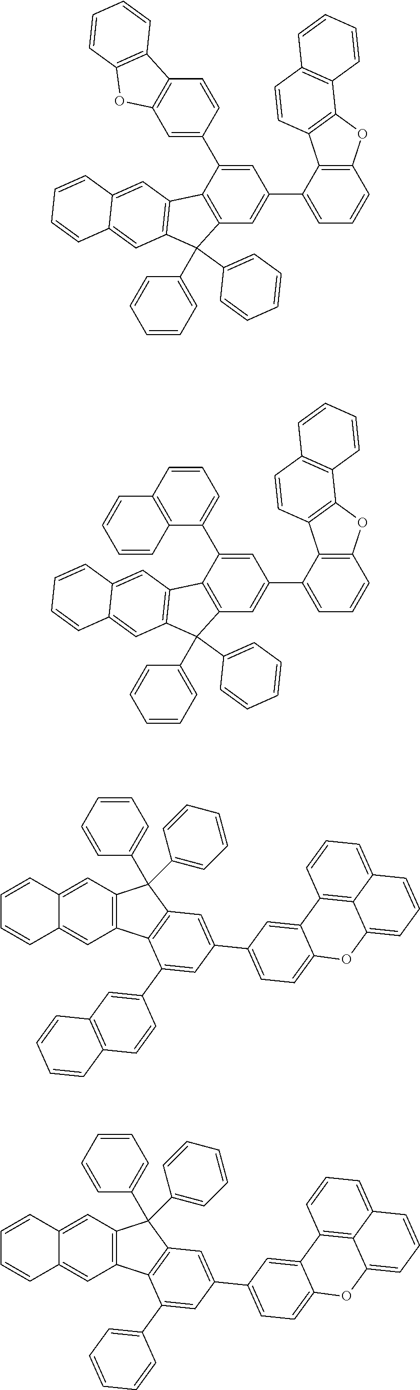 [SMILES: c1ccc(C2(c3ccccc3)c3cc4ccccc4cc3-c3c(-c4ccc5c(c4)oc4ccccc45)cc(-c4cccc5oc6c7ccccc7ccc6c45)cc32)cc1 ; c1ccc(C2(c3ccccc3)c3cc4ccccc4cc3-c3c(-c4cccc5ccccc45)cc(-c4cccc5oc6c7ccccc7ccc6c45)cc32)cc1 ; c1ccc(C2(c3ccccc3)c3cc4ccccc4cc3-c3c(-c4ccc5ccccc5c4)cc(-c4ccc5c(c4)-c4cccc6cccc(c46)O5)cc32)cc1 ; c1ccc(-c2cc(-c3ccc4c(c3)-c3cccc5cccc(c35)O4)cc3c2-c2cc4ccccc4cc2C3(c2ccccc2)c2ccccc2)cc1]

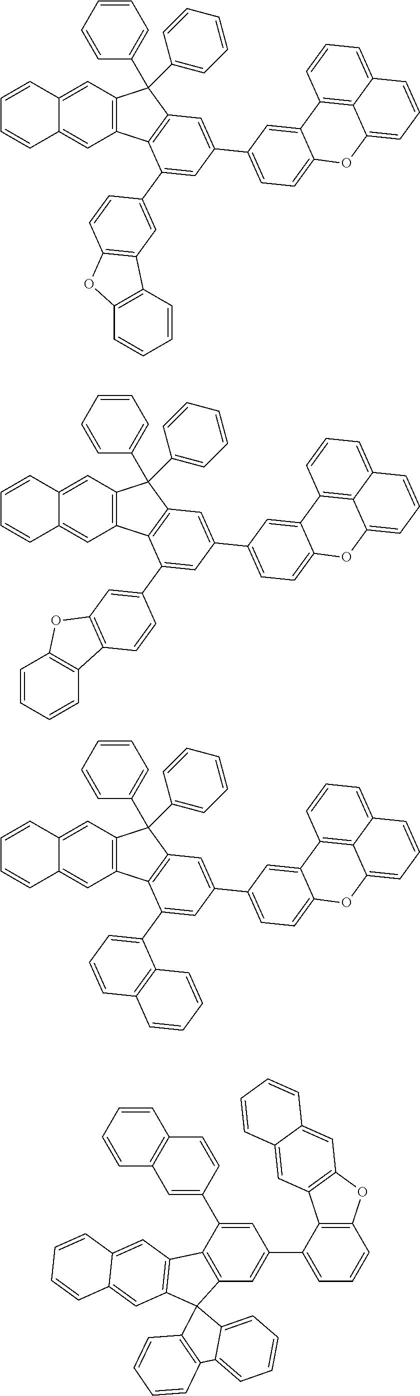

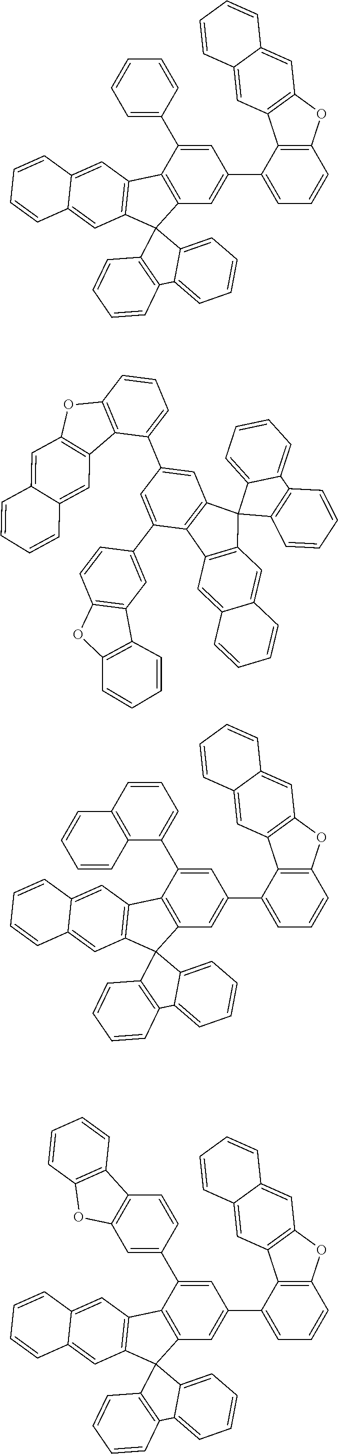 [SMILES: c1ccc(-c2cc(-c3cccc4oc5cc6ccccc6cc5c34)cc3c2-c2cc4ccccc4cc2C32c3ccccc3-c3ccccc32)cc1 ; c1ccc2c(c1)-c1ccccc1C21c2cc3ccccc3cc2-c2c(-c3ccc4oc5ccccc5c4c3)cc(-c3cccc4oc5cc6ccccc6cc5c34)cc21 ; c1ccc2c(c1)-c1ccccc1C21c2cc3ccccc3cc2-c2c(-c3cccc4ccccc34)cc(-c3cccc4oc5cc6ccccc6cc5c34)cc21 ; c1ccc2c(c1)-c1ccccc1C21c2cc3ccccc3cc2-c2c(-c3ccc4c(c3)oc3ccccc34)cc(-c3cccc4oc5cc6ccccc6cc5c34)cc21]

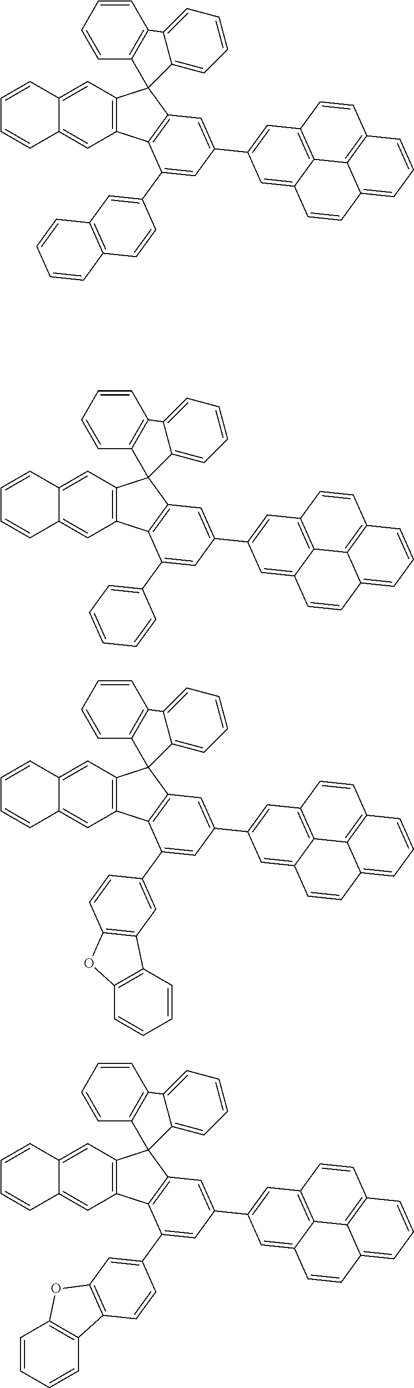

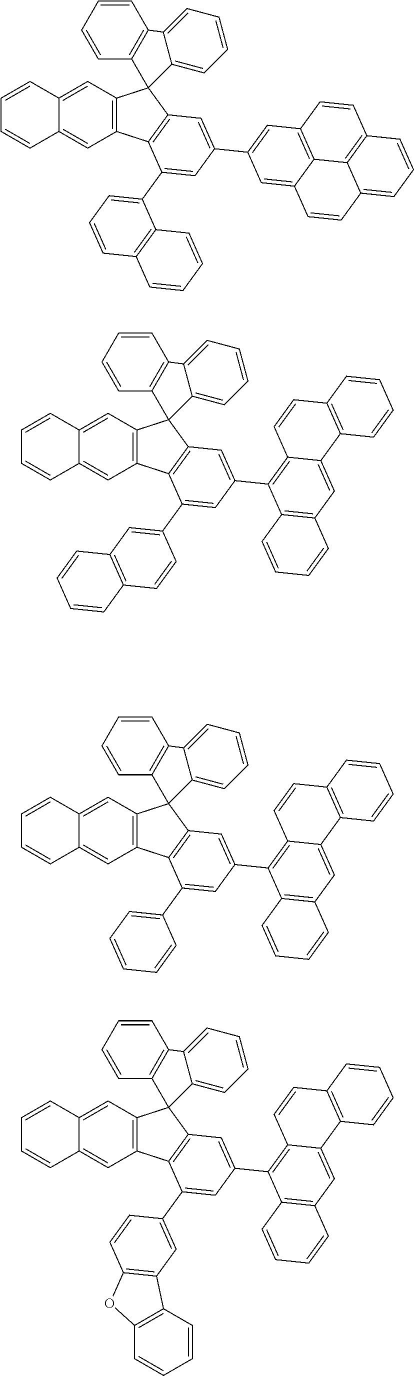

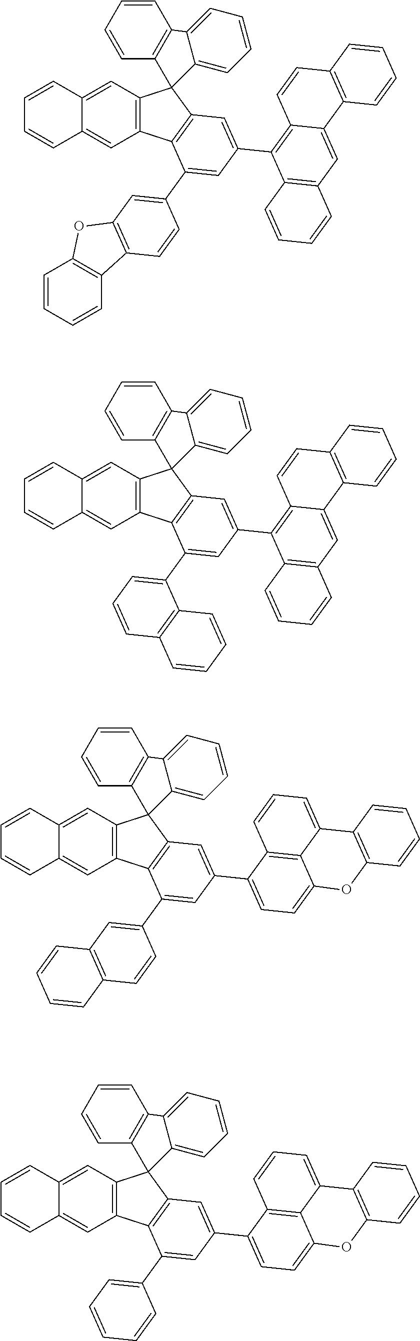

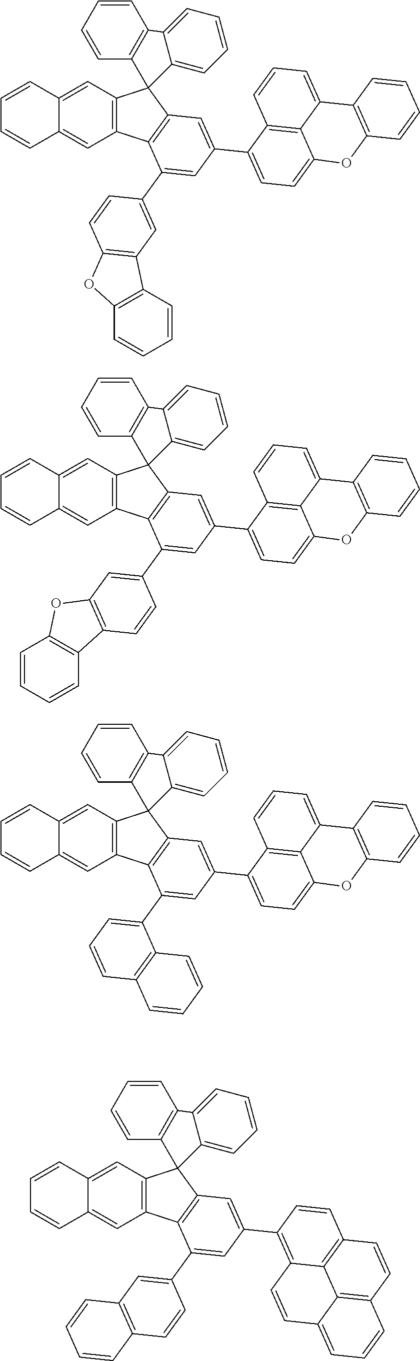

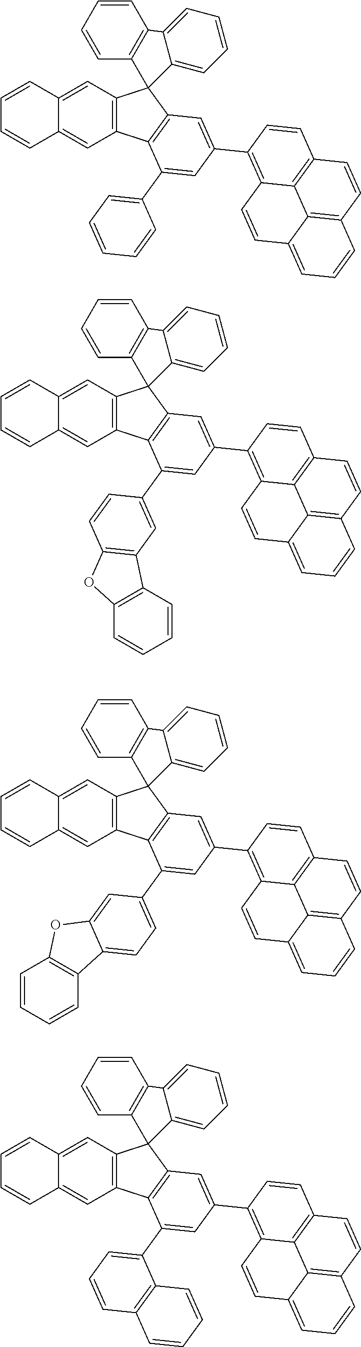

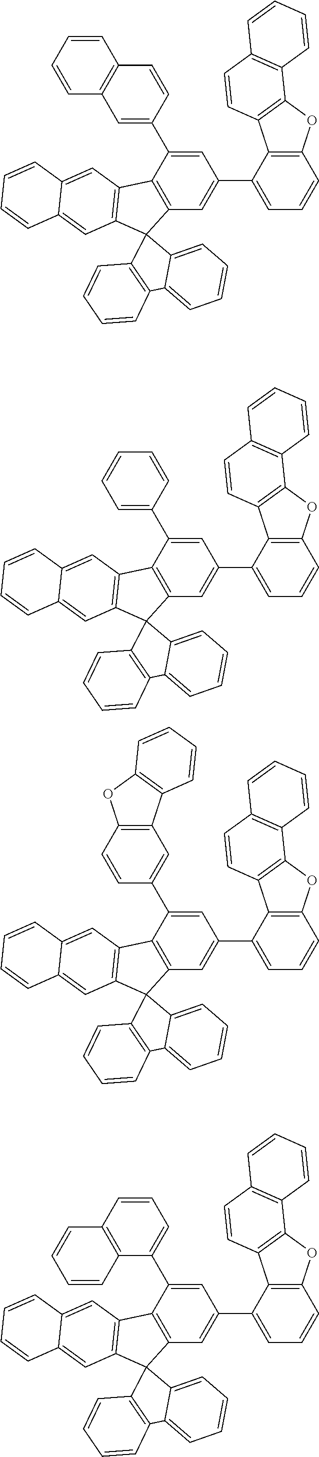

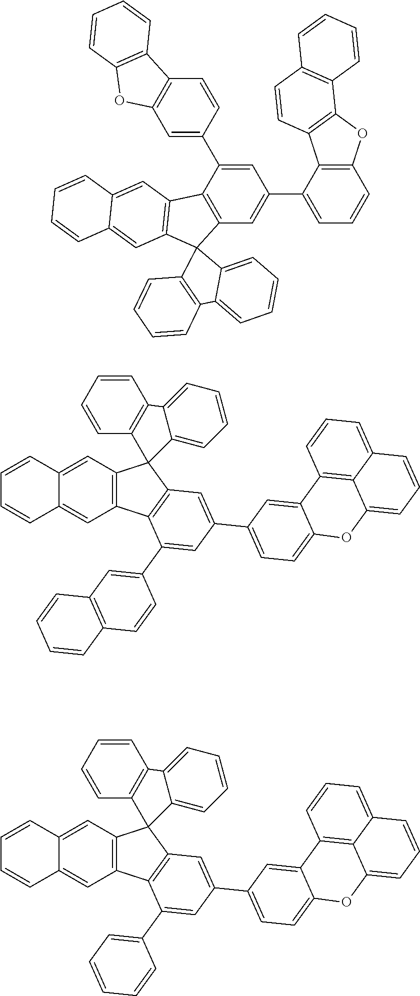

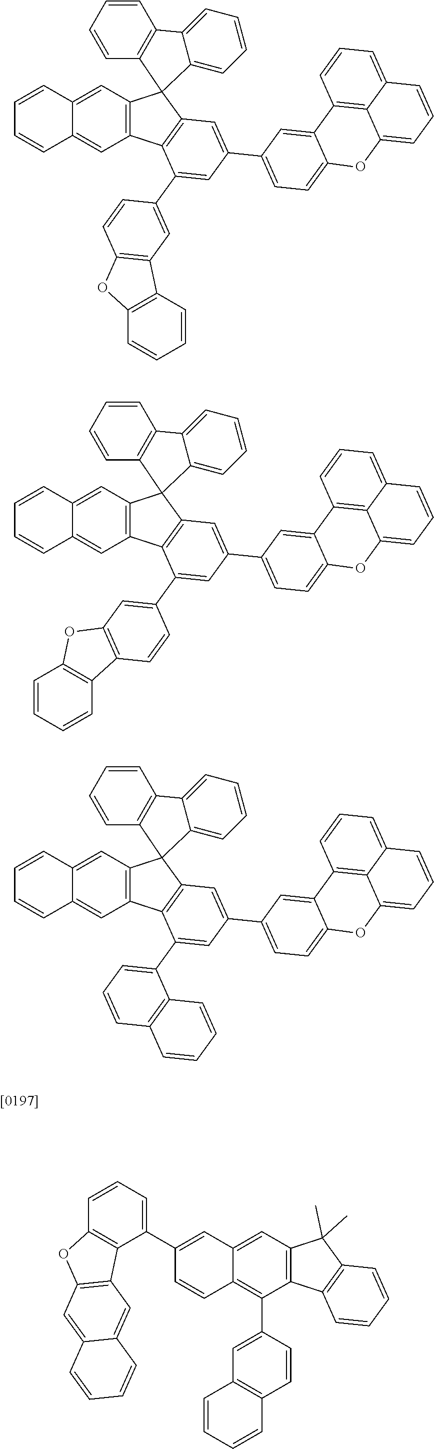

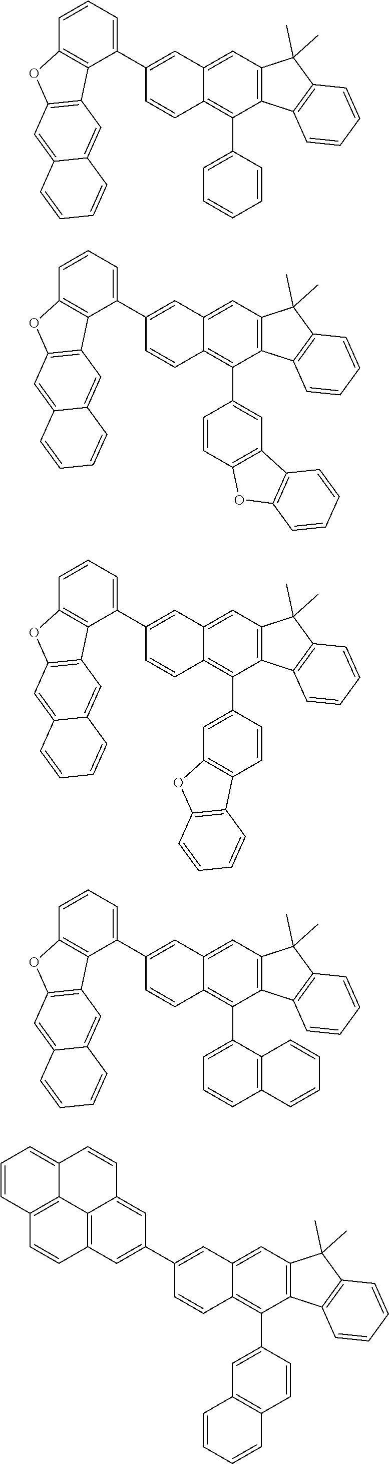

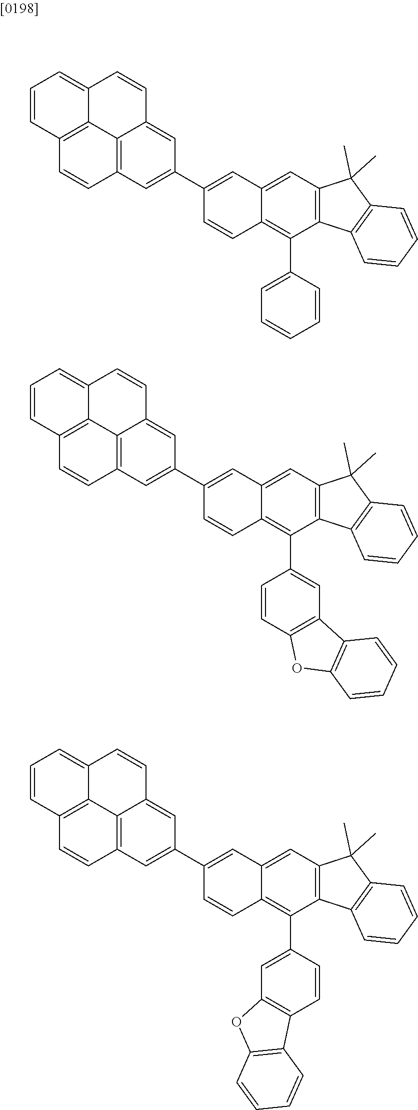

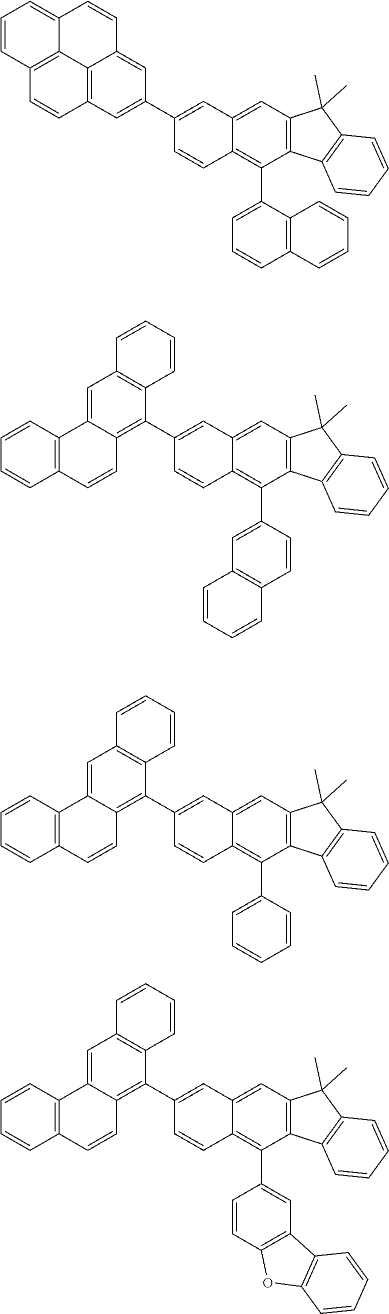

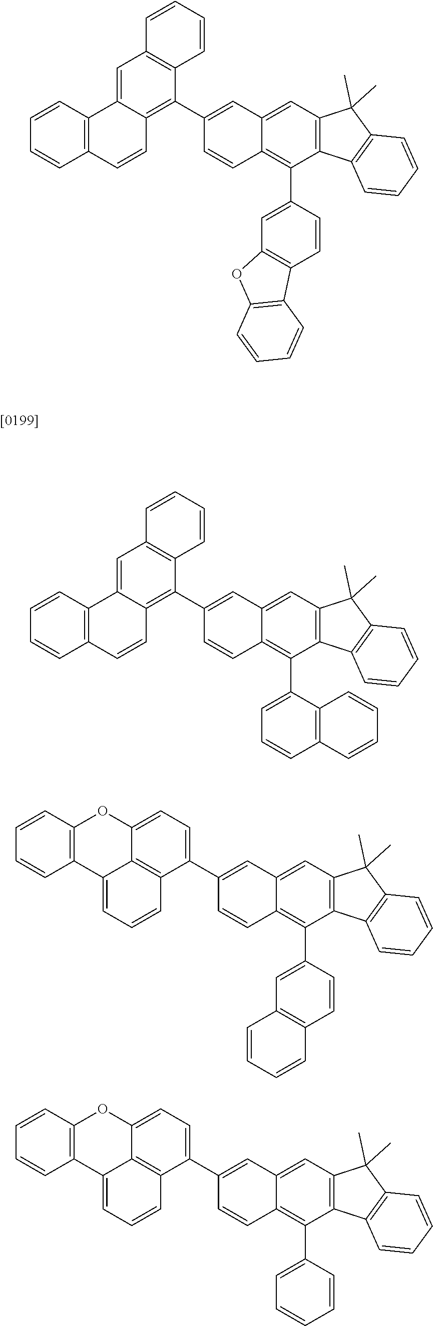

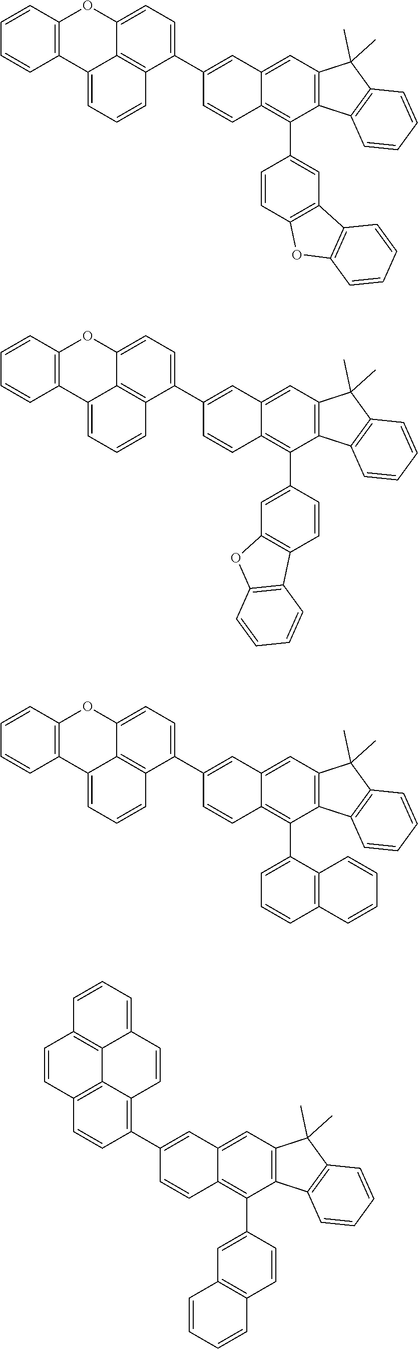

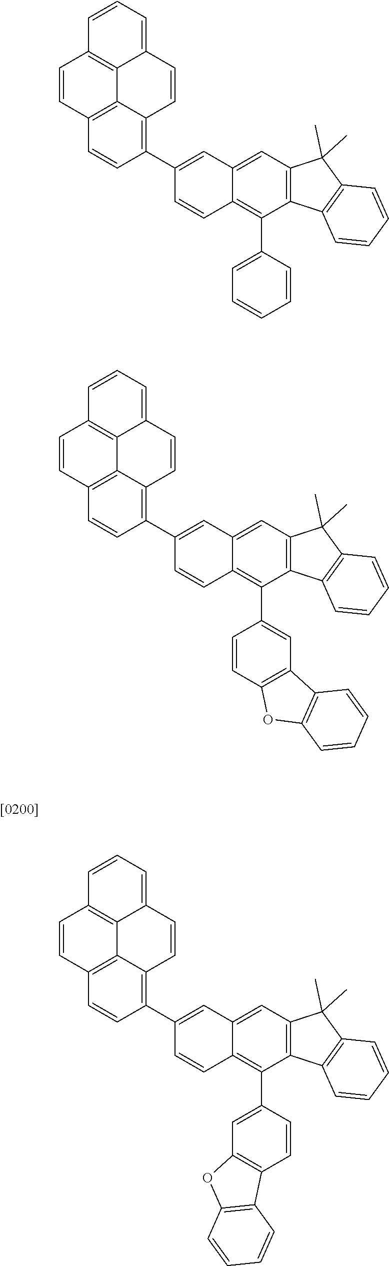

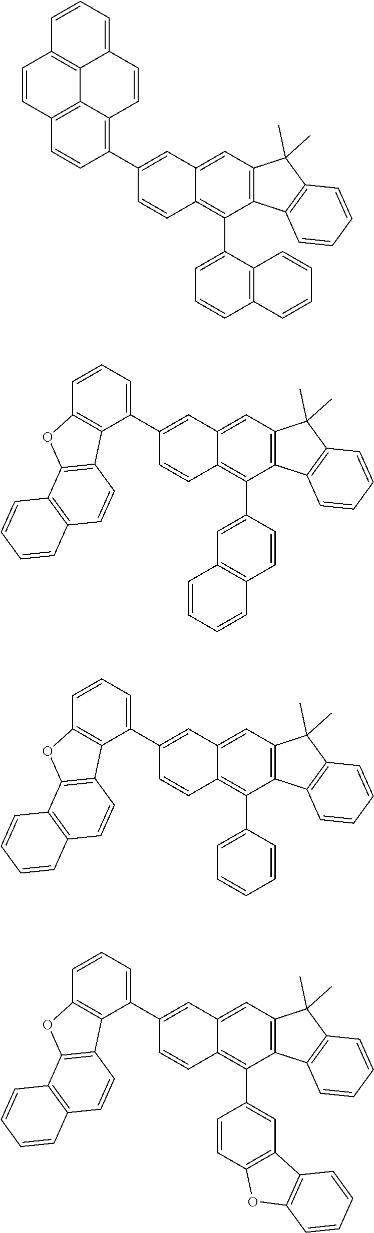

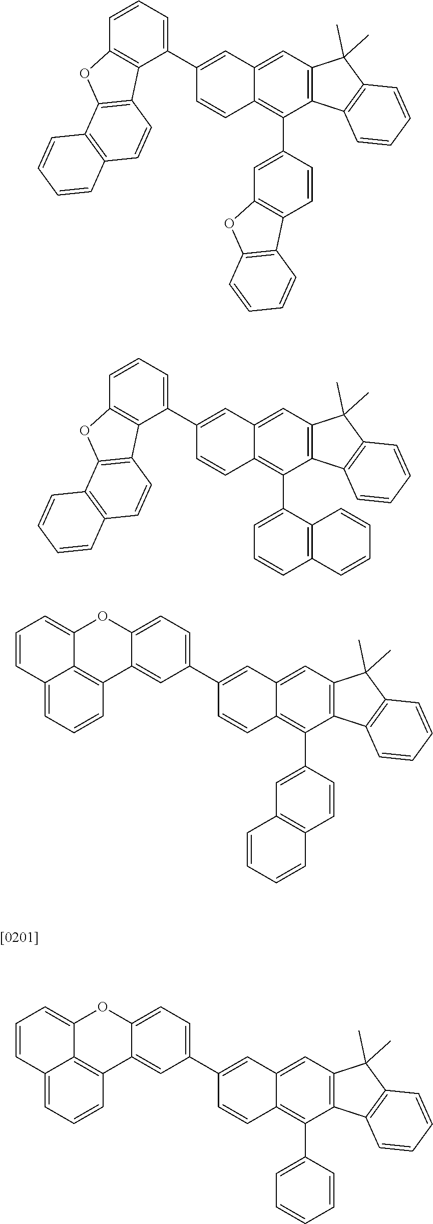 [SMILES: CC1(C)c2ccccc2-c2c1cc1cc(-c3cccc4oc5c6ccccc6ccc5c34)ccc1c2-c1ccc2c(c1)oc1ccccc12 ; CC1(C)c2ccccc2-c2c1cc1cc(-c3cccc4oc5c6ccccc6ccc5c34)ccc1c2-c1cccc2ccccc12 ; CC1(C)c2ccccc2-c2c1cc1cc(-c3ccc4c(c3)-c3cccc5cccc(c35)O4)ccc1c2-c1ccc2ccccc2c1 ; CC1(C)c2ccccc2-c2c1cc1cc(-c3ccc4c(c3)-c3cccc5cccc(c35)O4)ccc1c2-c1ccccc1]

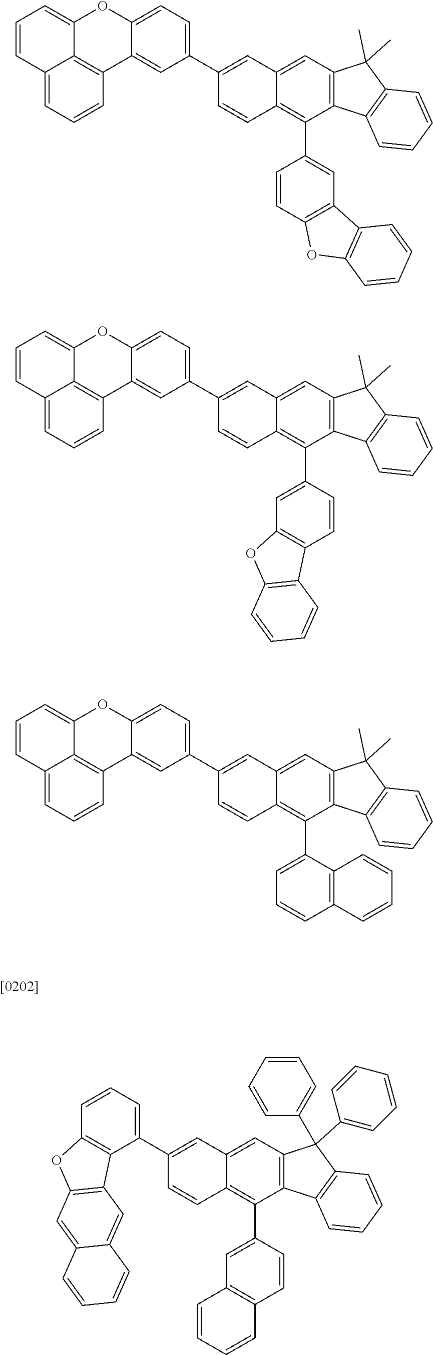 [SMILES: CC1(C)c2ccccc2-c2c1cc1cc(-c3ccc4c(c3)-c3cccc5cccc(c35)O4)ccc1c2-c1ccc2oc3ccccc3c2c1 ; CC1(C)c2ccccc2-c2c1cc1cc(-c3ccc4c(c3)-c3cccc5cccc(c35)O4)ccc1c2-c1ccc2c(c1)oc1ccccc12 ; CC1(C)c2ccccc2-c2c1cc1cc(-c3ccc4c(c3)-c3cccc5cccc(c35)O4)ccc1c2-c1cccc2ccccc12 ; c1ccc(C2(c3ccccc3)c3ccccc3-c3c2cc2cc(-c4cccc5oc6cc7ccccc7cc6c45)ccc2c3-c2ccc3ccccc3c2)cc1]

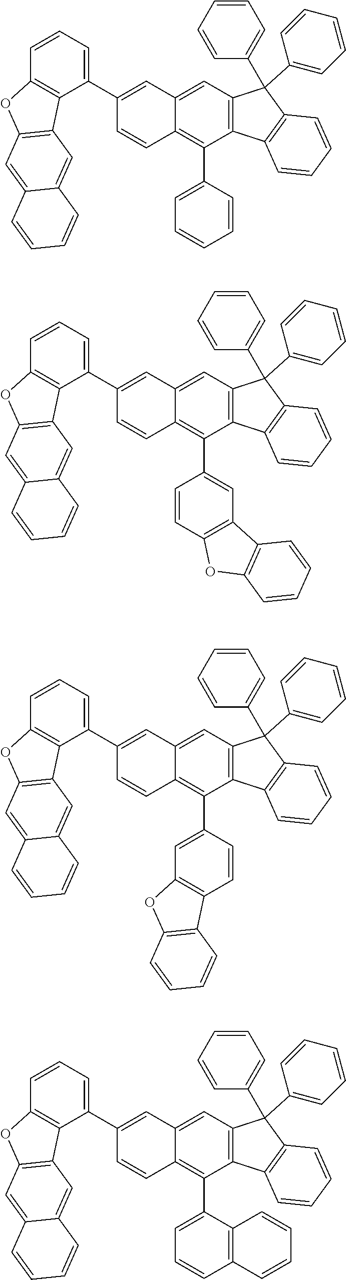

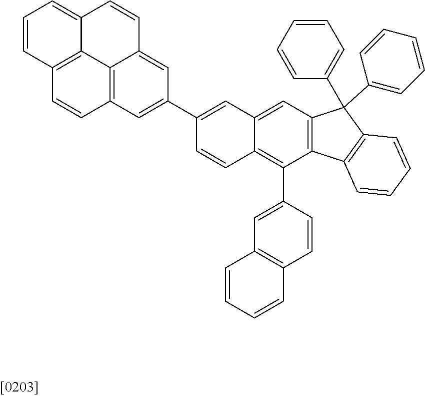 [SMILES: c1ccc(C2(c3ccccc3)c3ccccc3-c3c2cc2cc(-c4cc5ccc6cccc7ccc(c4)c5c67)ccc2c3-c2ccc3ccccc3c2)cc1]

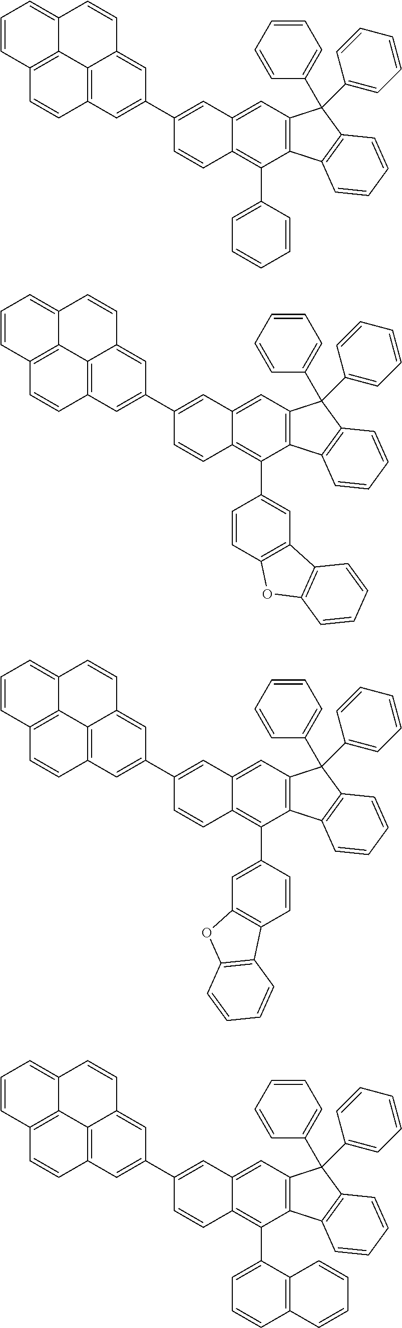

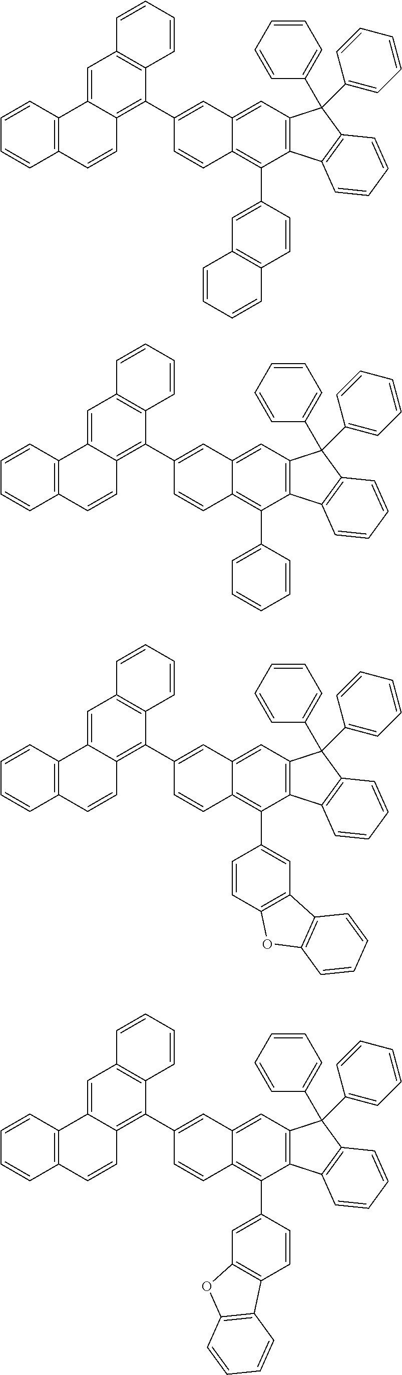

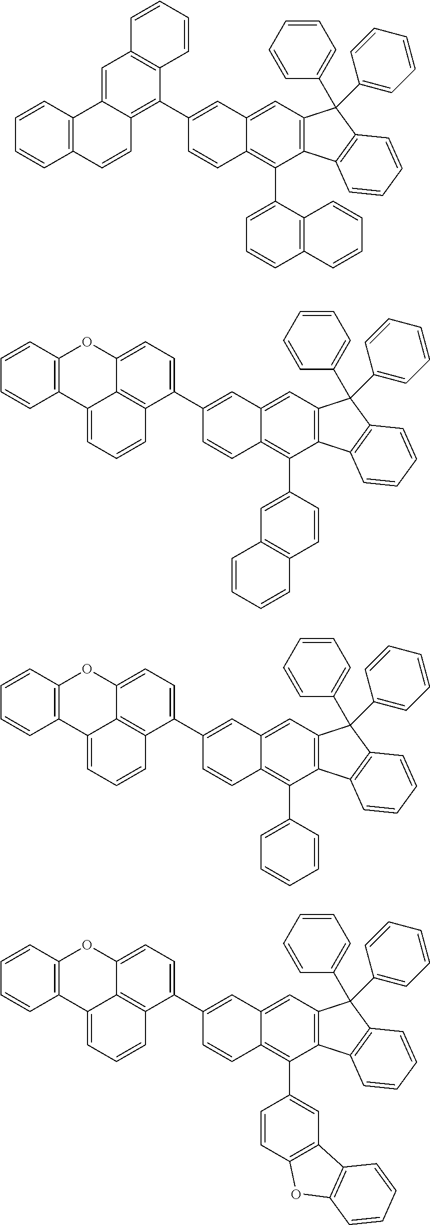

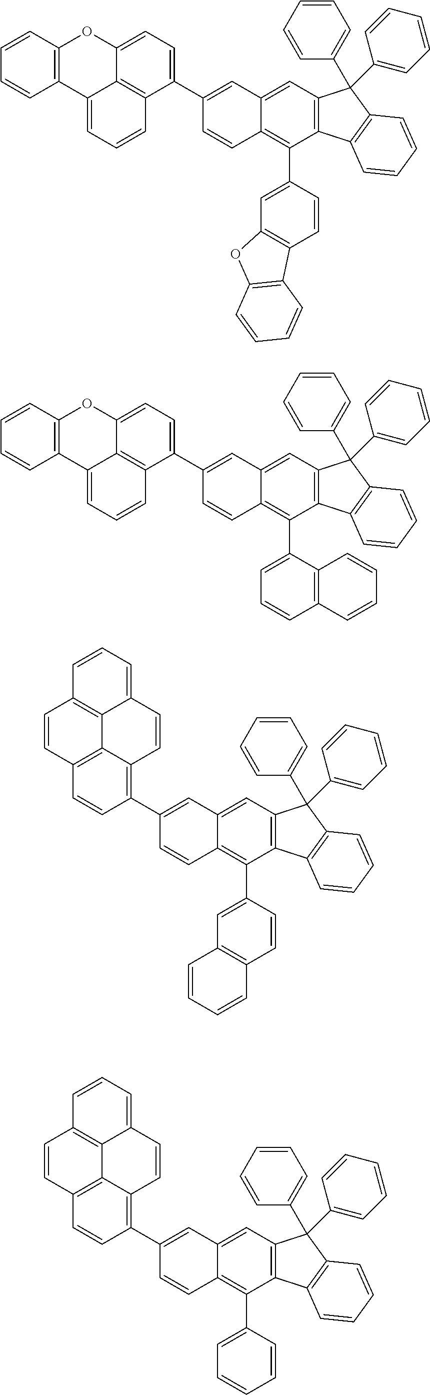

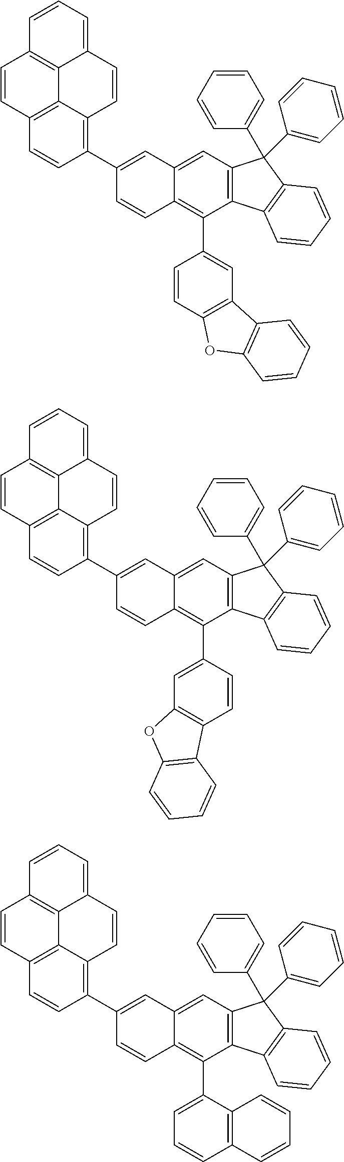

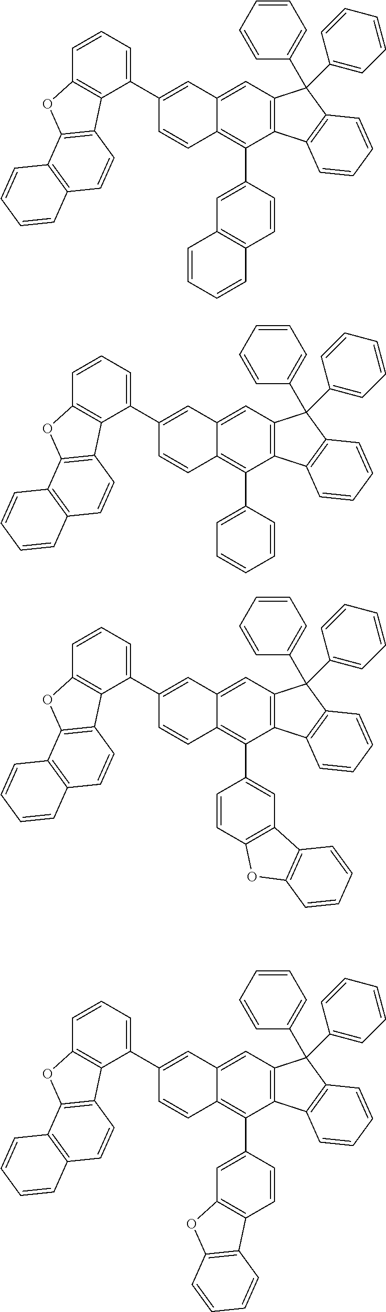

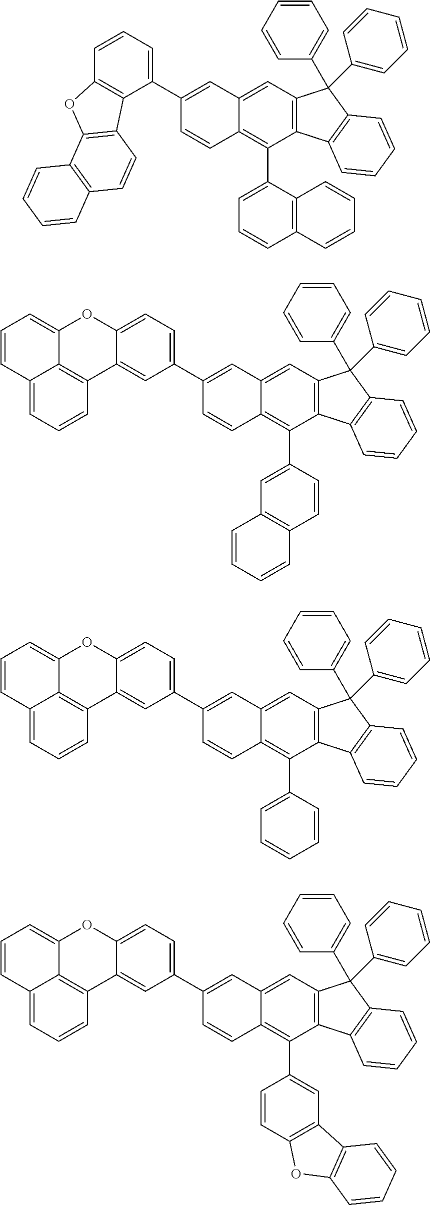

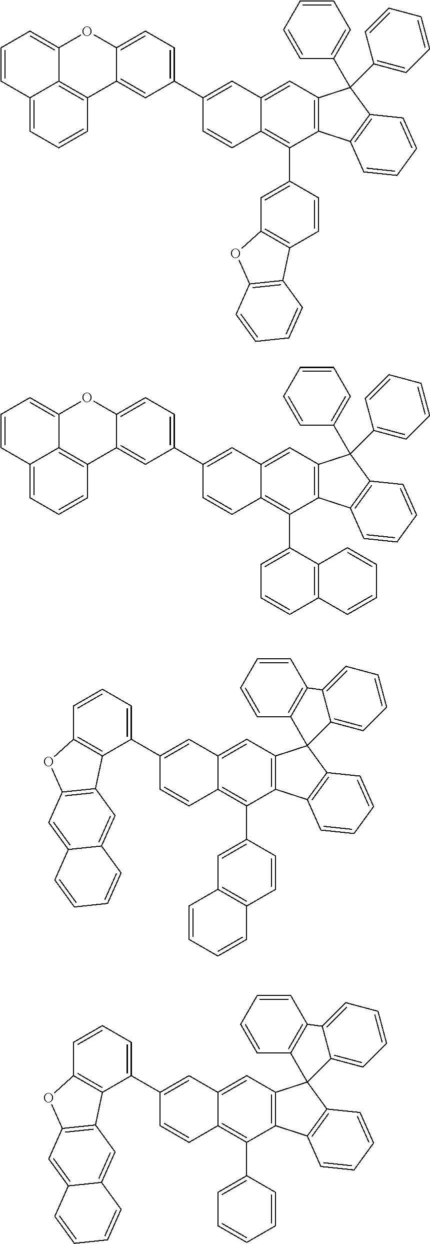

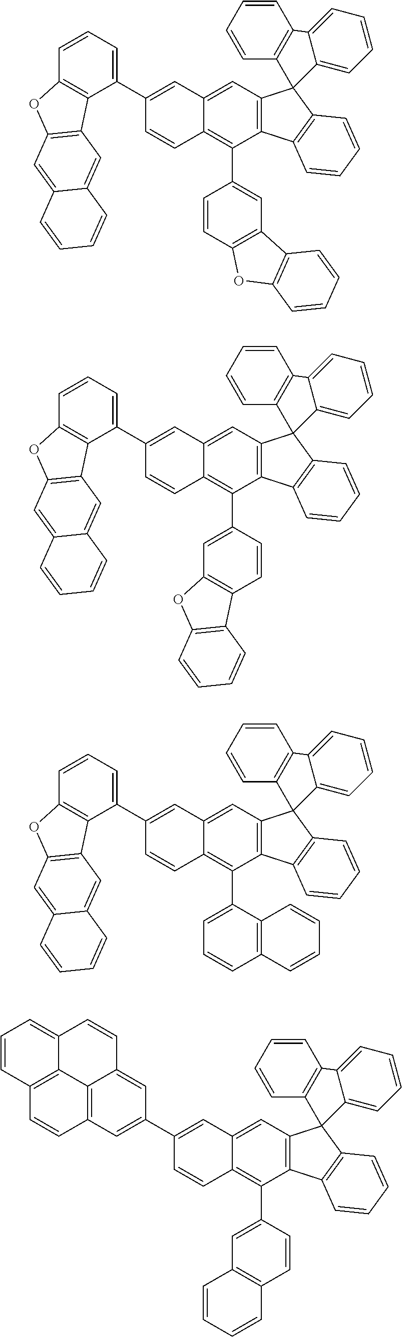

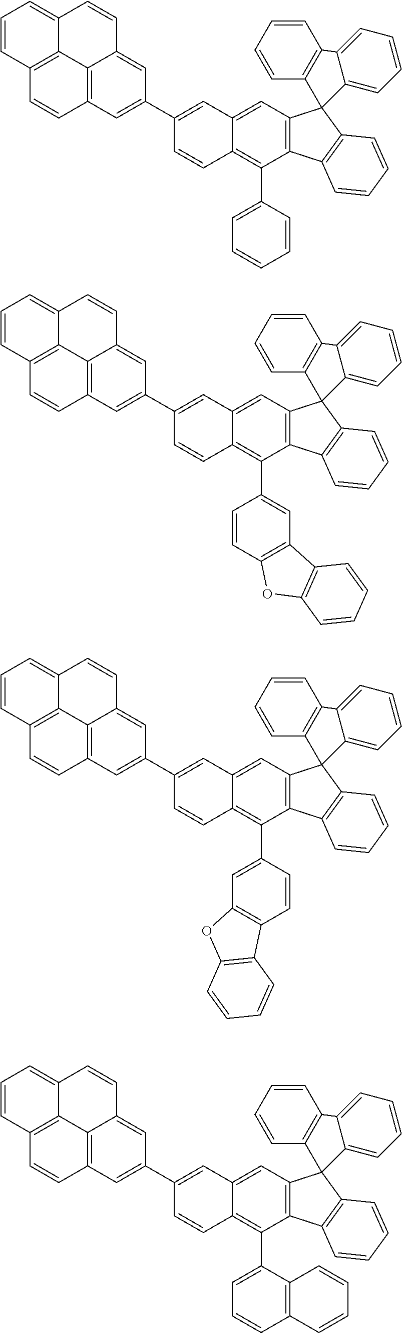

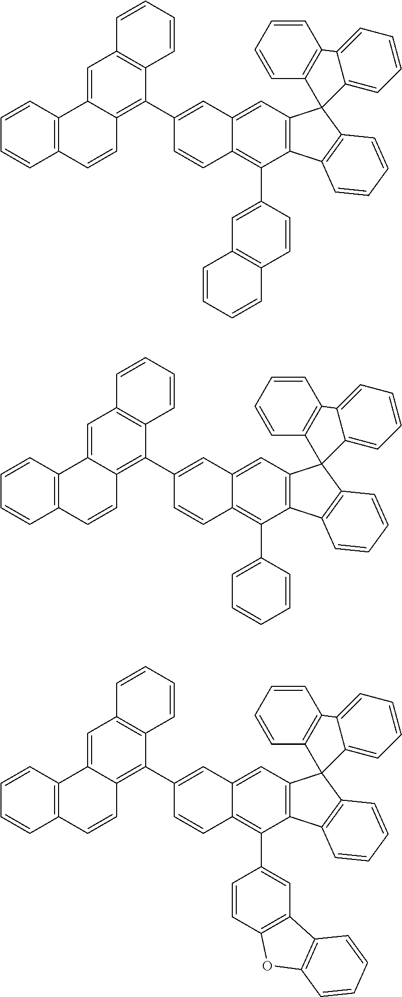

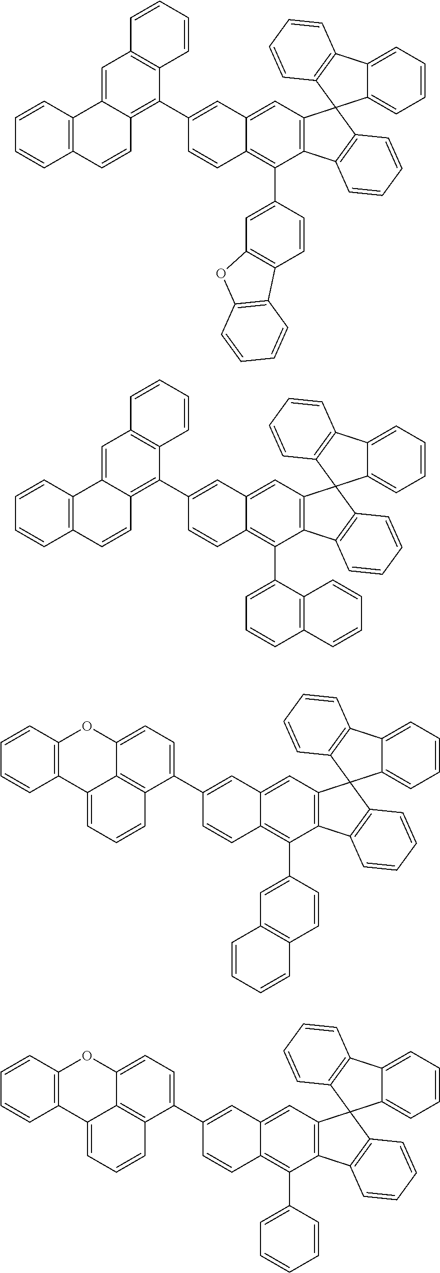

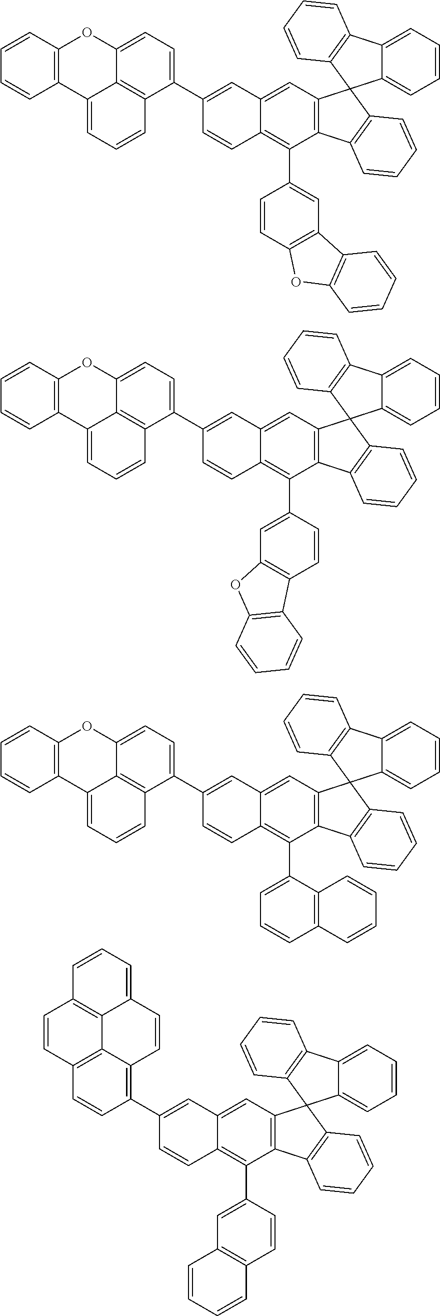

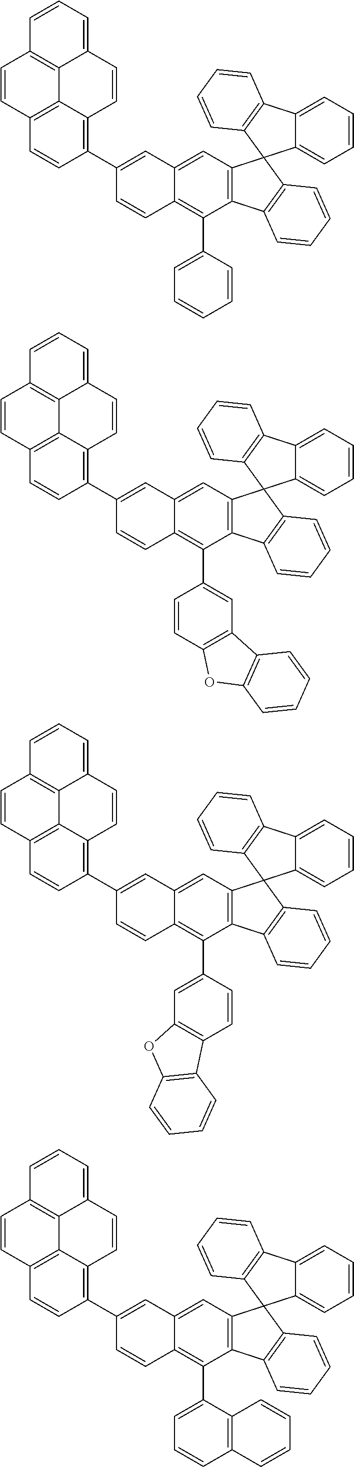

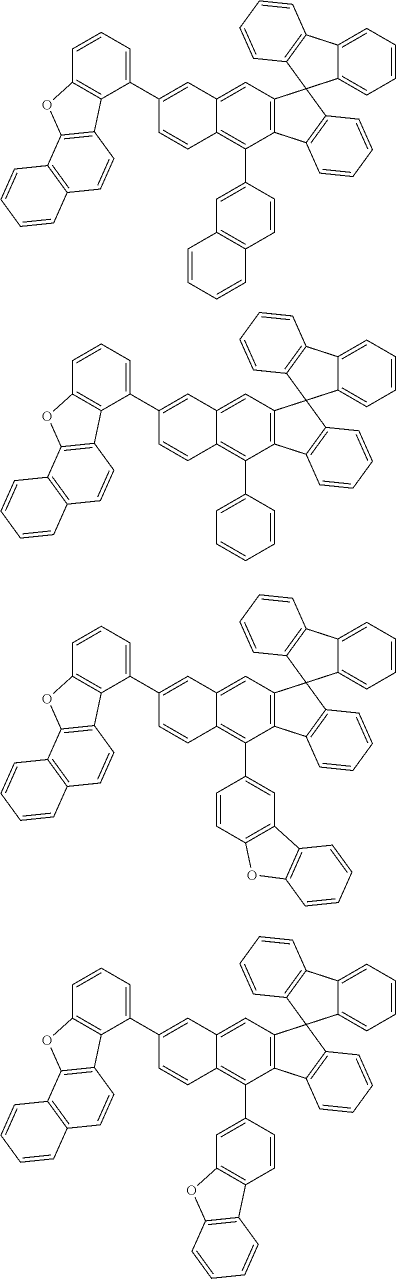

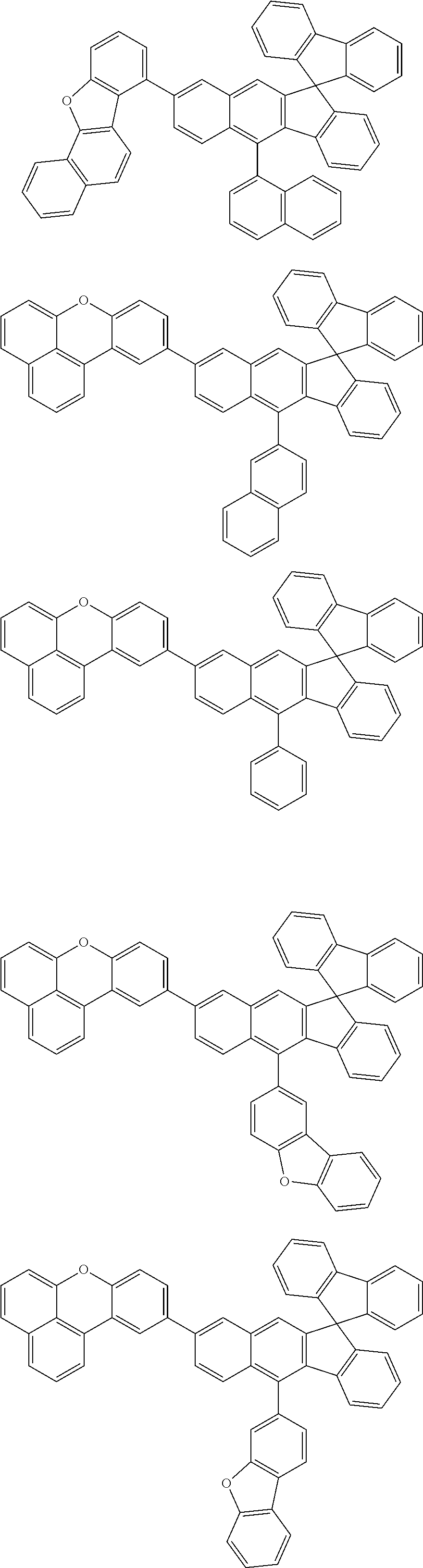

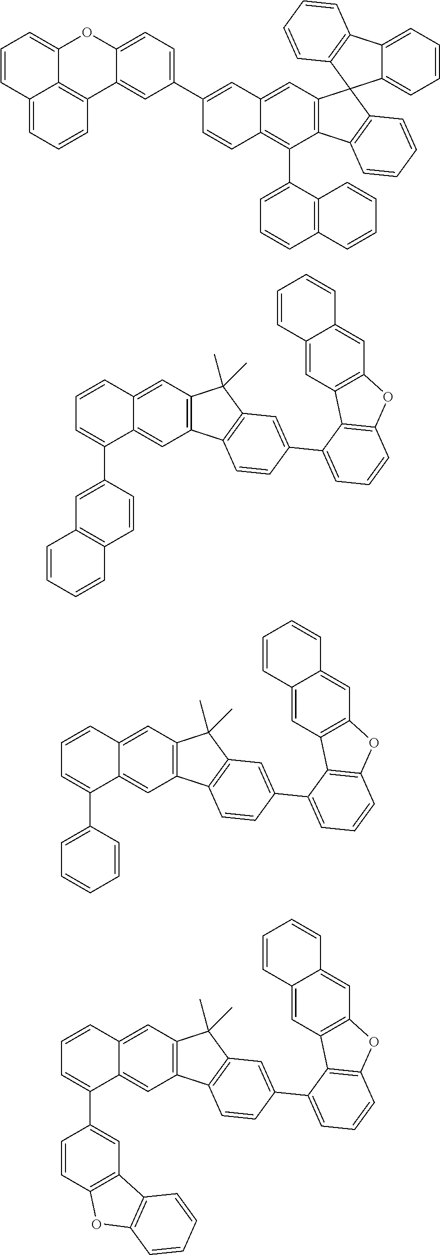

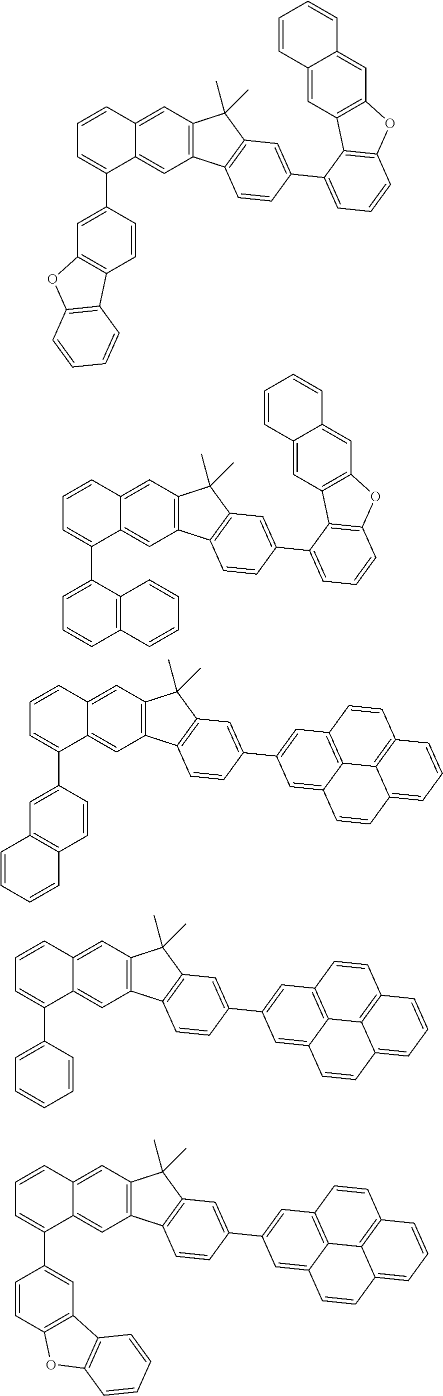

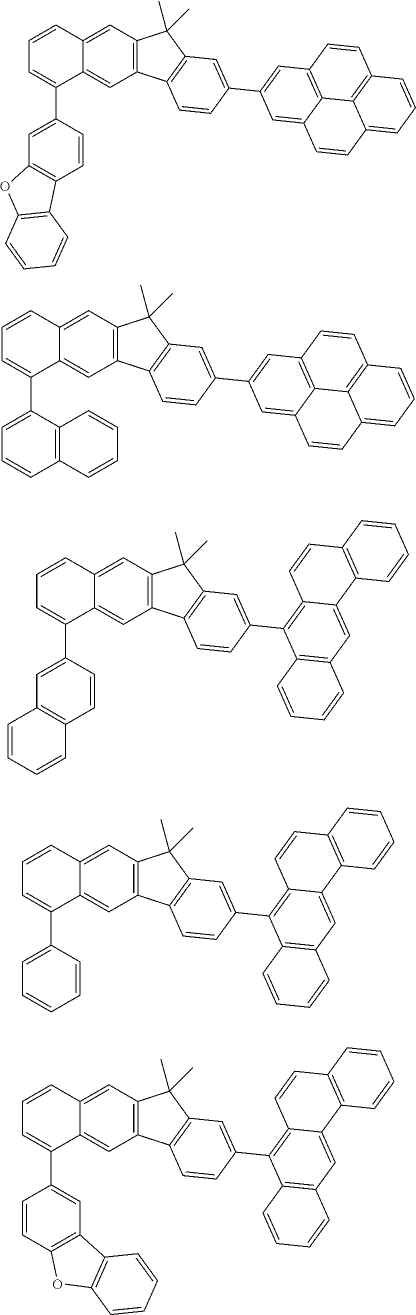

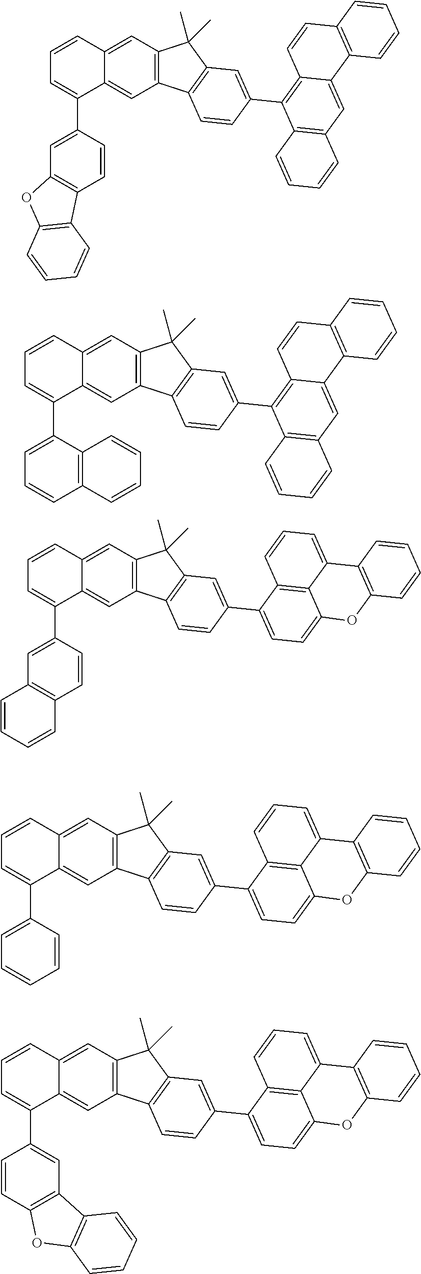

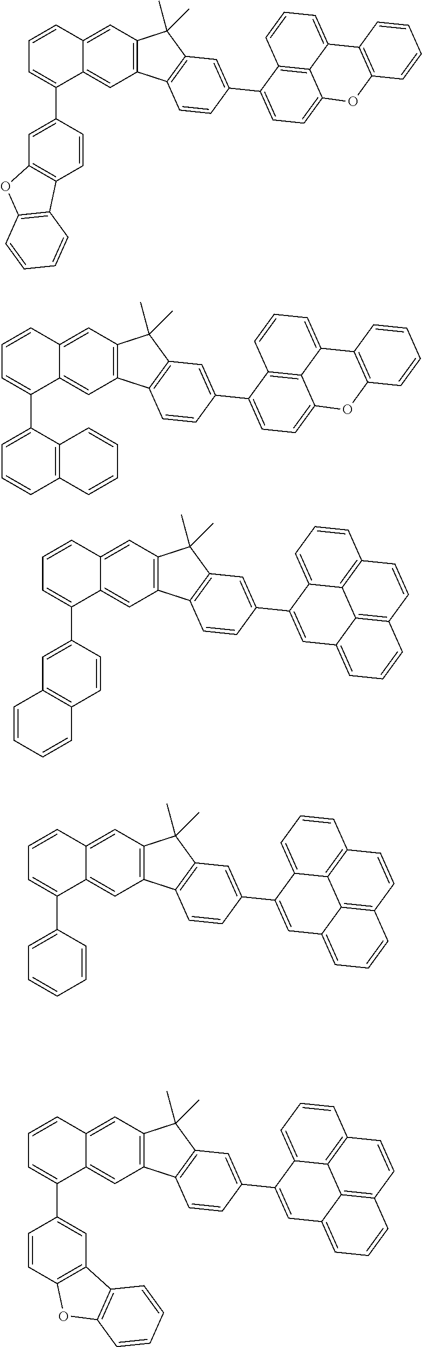

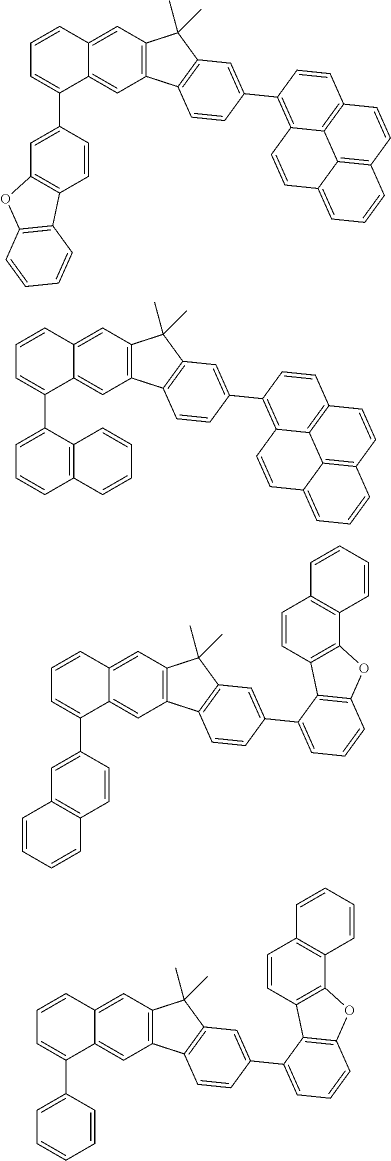

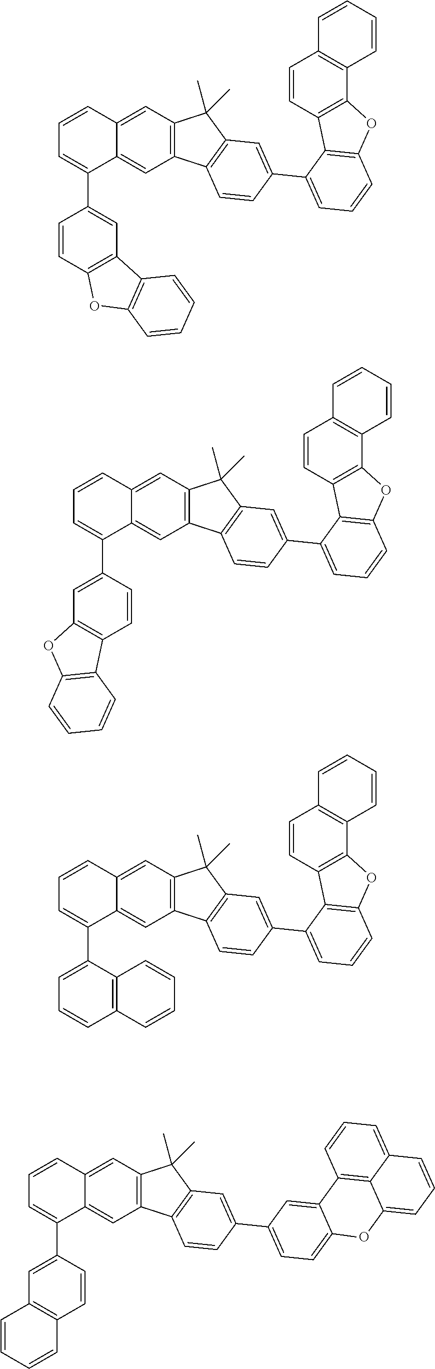 [SMILES: CC1(C)c2cc(-c3cccc4oc5c6ccccc6ccc5c34)ccc2-c2cc3c(-c4ccc5oc6ccccc6c5c4)cccc3cc21 ; CC1(C)c2cc(-c3cccc4oc5c6ccccc6ccc5c34)ccc2-c2cc3c(-c4ccc5c(c4)oc4ccccc45)cccc3cc21 ; CC1(C)c2cc(-c3cccc4oc5c6ccccc6ccc5c34)ccc2-c2cc3c(-c4cccc5ccccc45)cccc3cc21 ; CC1(C)c2cc(-c3ccc4c(c3)-c3cccc5cccc(c35)O4)ccc2-c2cc3c(-c4ccc5ccccc5c4)cccc3cc21]

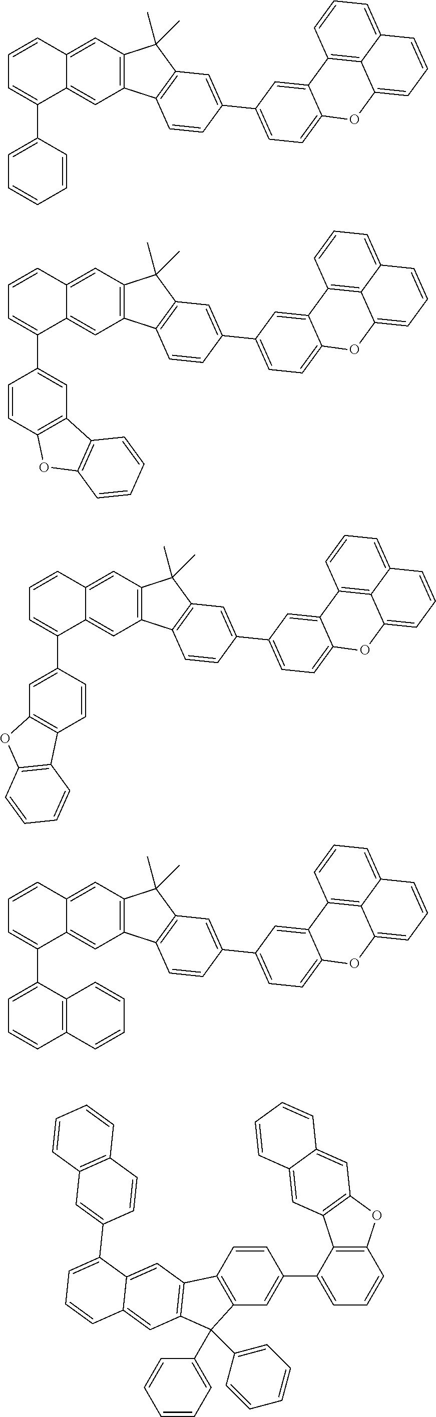 [SMILES: CC1(C)c2cc(-c3ccc4c(c3)-c3cccc5cccc(c35)O4)ccc2-c2cc3c(-c4ccccc4)cccc3cc21 ; CC1(C)c2cc(-c3ccc4c(c3)-c3cccc5cccc(c35)O4)ccc2-c2cc3c(-c4ccc5oc6ccccc6c5c4)cccc3cc21 ; CC1(C)c2cc(-c3ccc4c(c3)-c3cccc5cccc(c35)O4)ccc2-c2cc3c(-c4ccc5c(c4)oc4ccccc45)cccc3cc21 ; CC1(C)c2cc(-c3ccc4c(c3)-c3cccc5cccc(c35)O4)ccc2-c2cc3c(-c4cccc5ccccc45)cccc3cc21 ; c1ccc(C2(c3ccccc3)c3cc(-c4cccc5oc6cc7ccccc7cc6c45)ccc3-c3cc4c(-c5ccc6ccccc6c5)cccc4cc32)cc1]

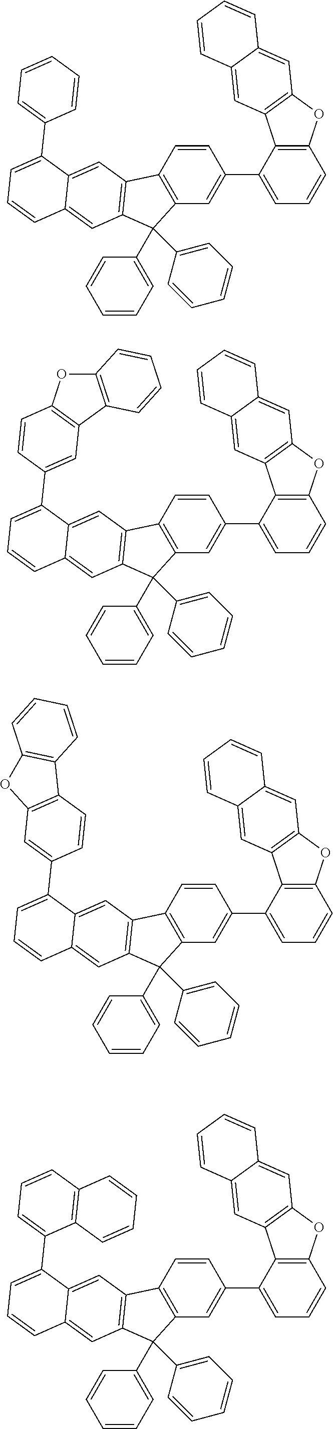

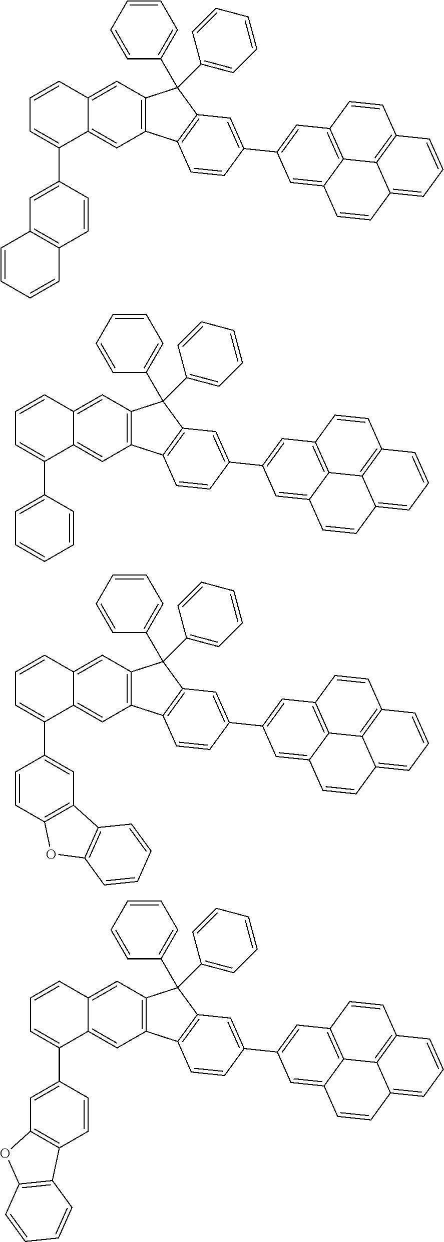

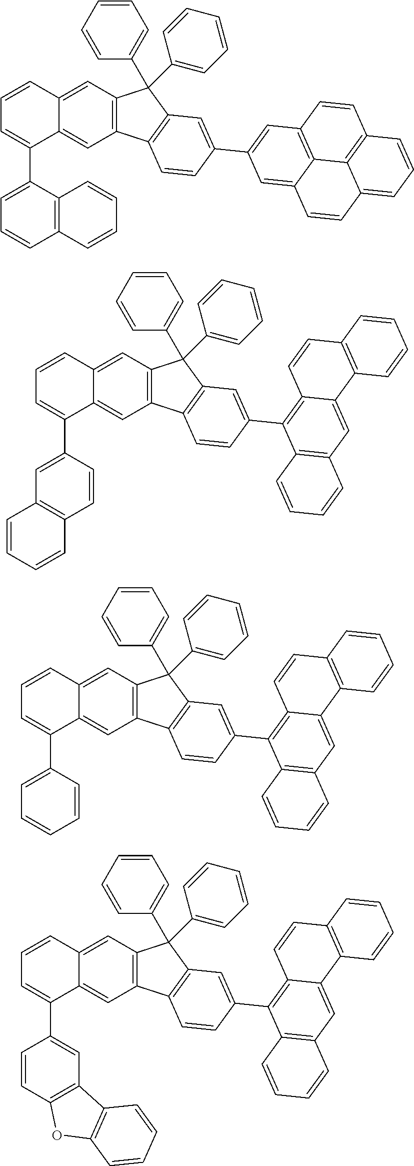

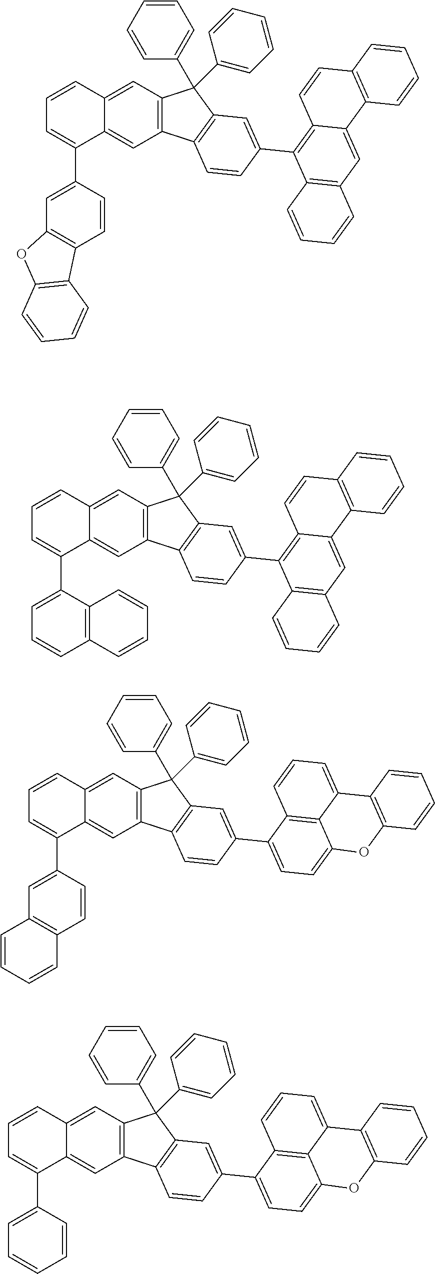

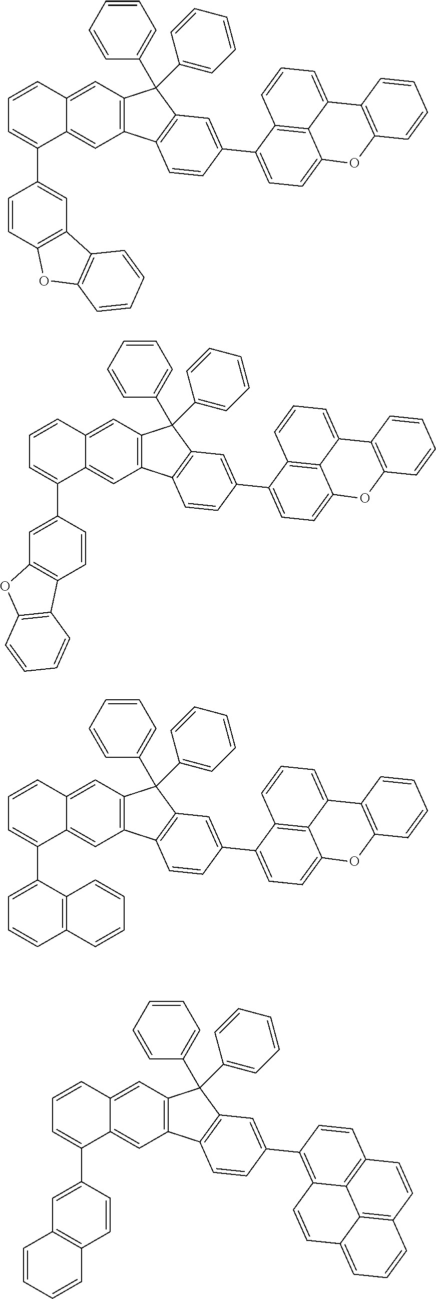

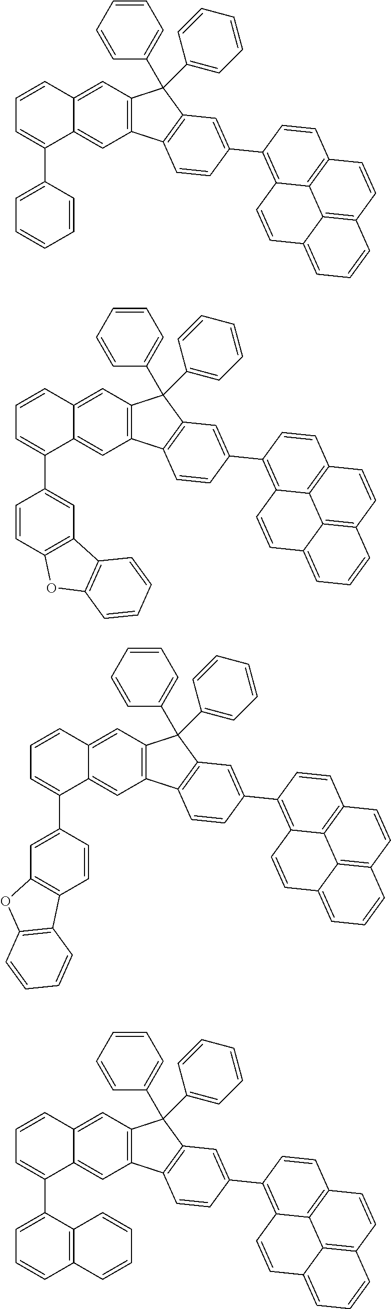

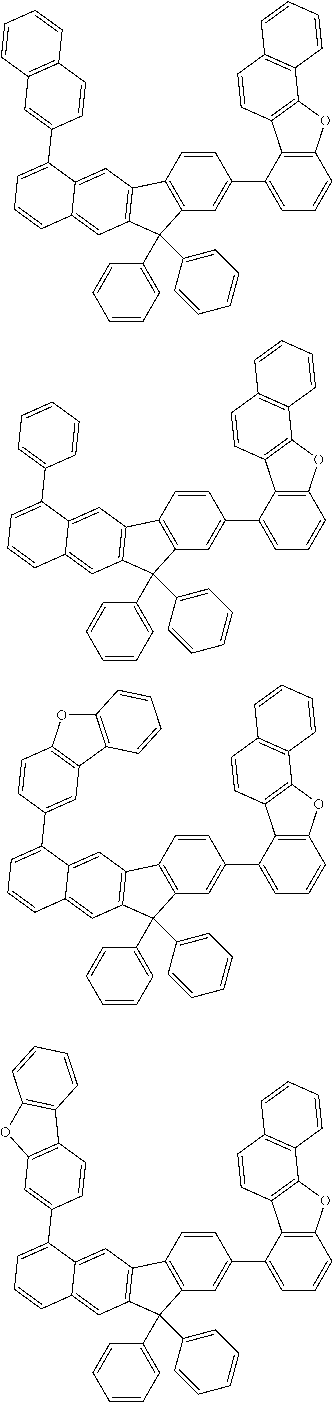

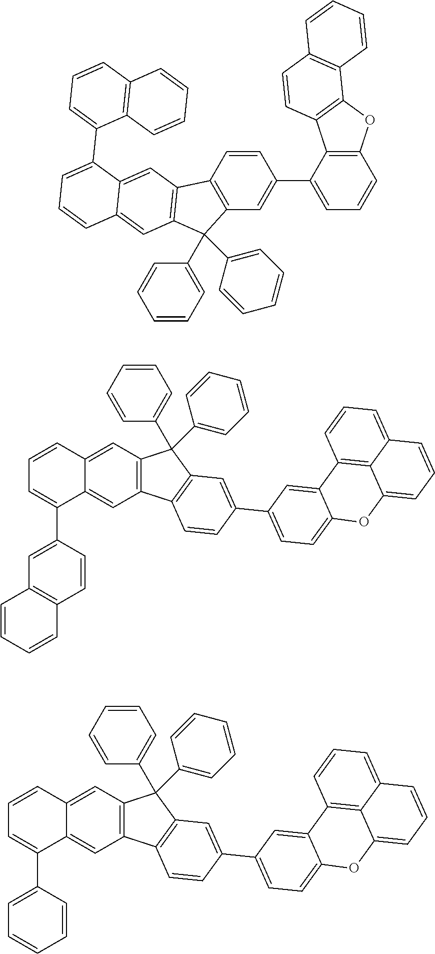

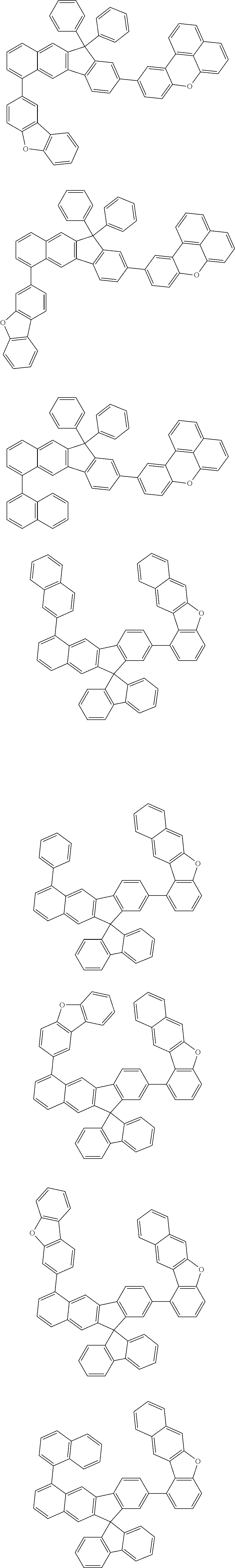

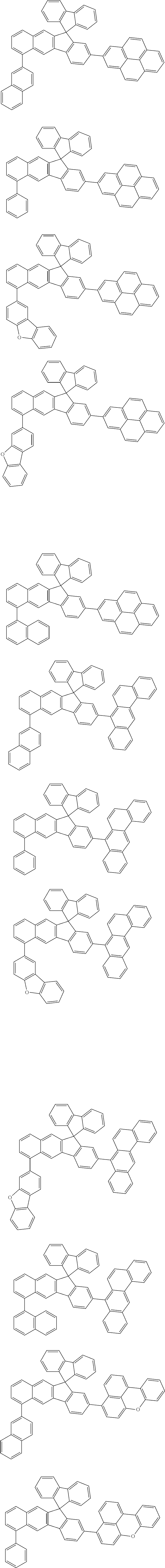

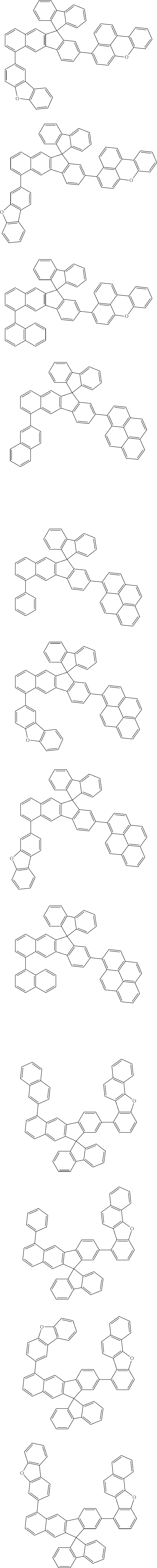

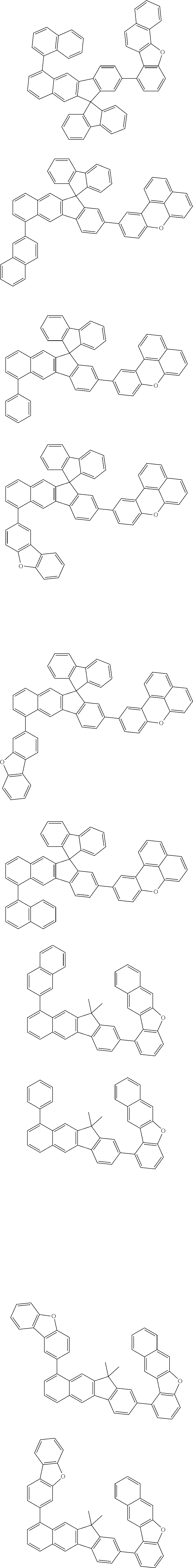

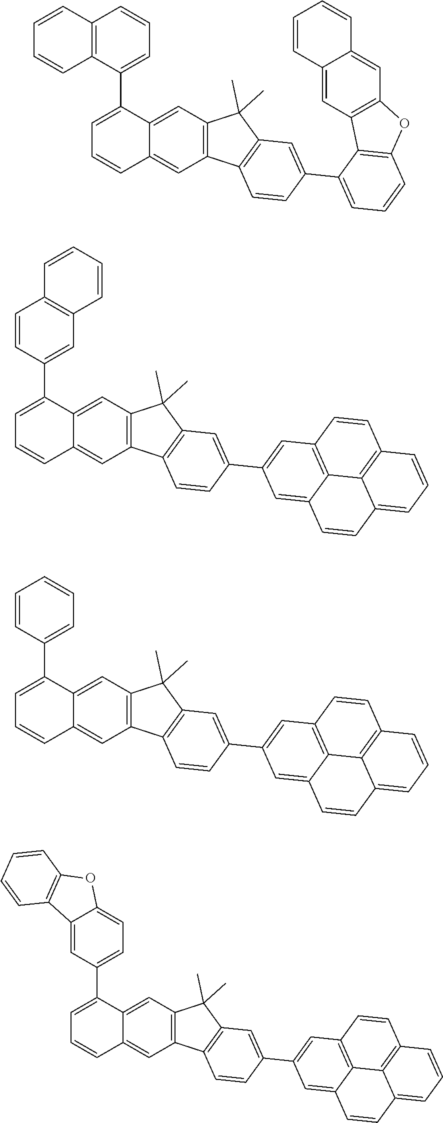

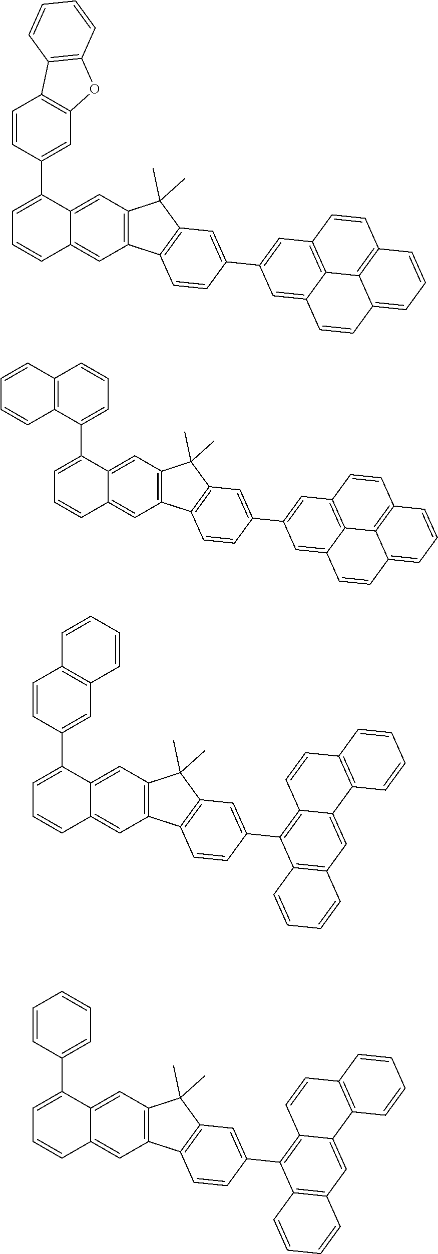

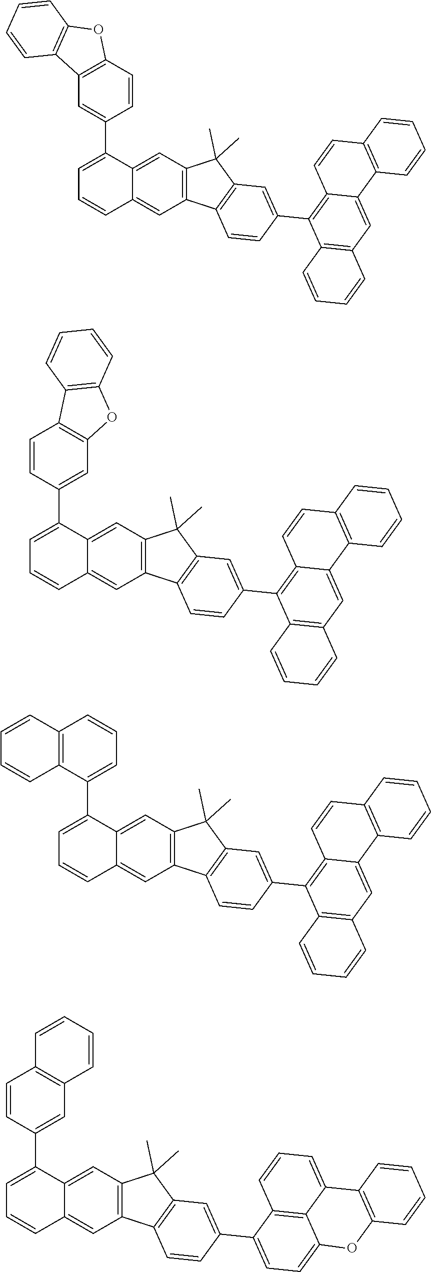

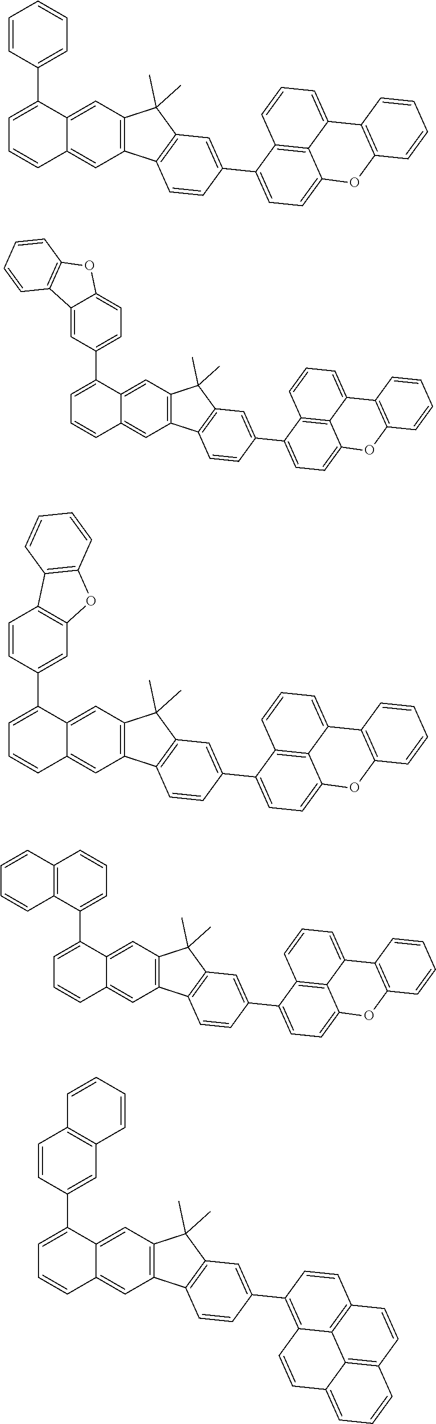

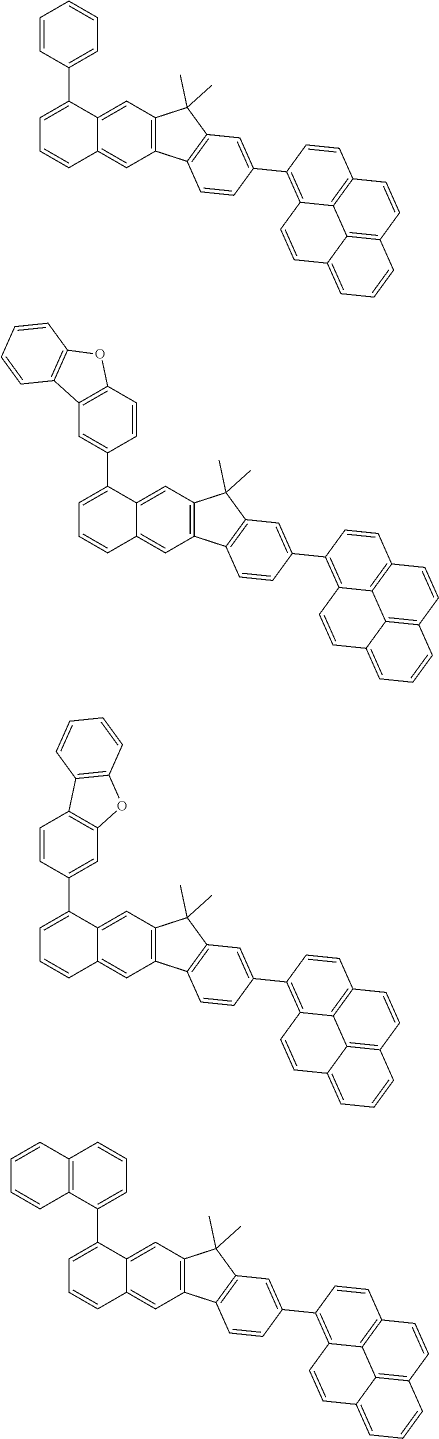

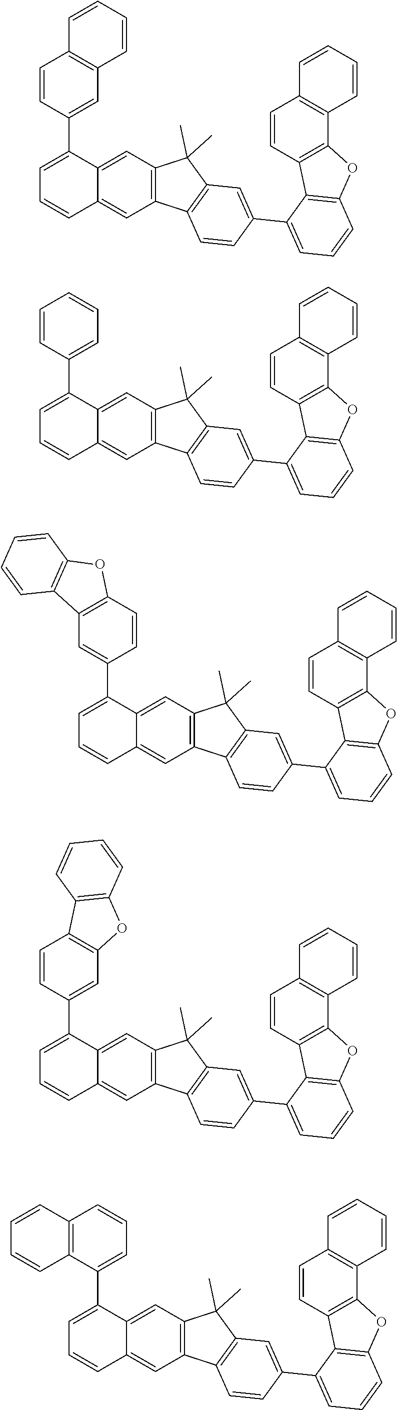

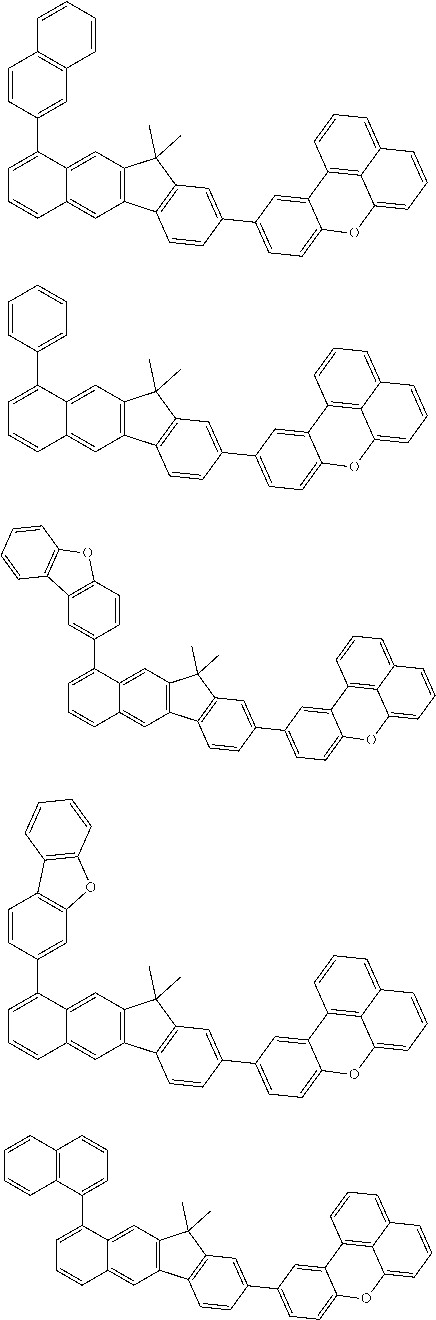 [SMILES: CC1(C)c2cc(-c3ccc4c(c3)-c3cccc5cccc(c35)O4)ccc2-c2cc3cccc(-c4ccc5ccccc5c4)c3cc21 ; CC1(C)c2cc(-c3ccc4c(c3)-c3cccc5cccc(c35)O4)ccc2-c2cc3cccc(-c4ccccc4)c3cc21 ; CC1(C)c2cc(-c3ccc4c(c3)-c3cccc5cccc(c35)O4)ccc2-c2cc3cccc(-c4ccc5oc6ccccc6c5c4)c3cc21 ; CC1(C)c2cc(-c3ccc4c(c3)-c3cccc5cccc(c35)O4)ccc2-c2cc3cccc(-c4ccc5c(c4)oc4ccccc45)c3cc21 ; CC1(C)c2cc(-c3ccc4c(c3)-c3cccc5cccc(c35)O4)ccc2-c2cc3cccc(-c4cccc5ccccc45)c3cc21]

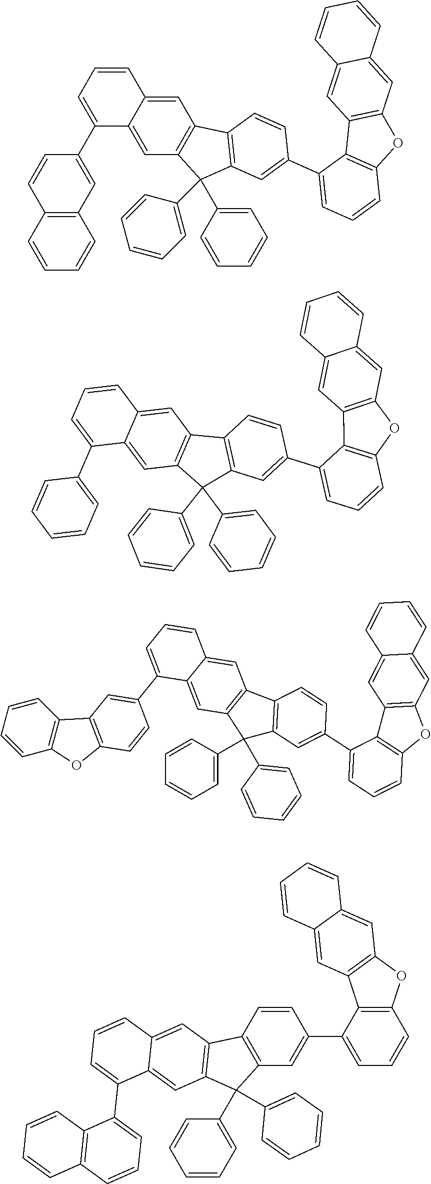 [SMILES: c1ccc(C2(c3ccccc3)c3cc(-c4cccc5oc6cc7ccccc7cc6c45)ccc3-c3cc4cccc(-c5ccc6ccccc6c5)c4cc32)cc1 ; c1ccc(-c2cccc3cc4c(cc23)C(c2ccccc2)(c2ccccc2)c2cc(-c3cccc5oc6cc7ccccc7cc6c35)ccc2-4)cc1 ; c1ccc(C2(c3ccccc3)c3cc(-c4cccc5oc6cc7ccccc7cc6c45)ccc3-c3cc4cccc(-c5ccc6oc7ccccc7c6c5)c4cc32)cc1 ; c1ccc(C2(c3ccccc3)c3cc(-c4cccc5oc6cc7ccccc7cc6c45)ccc3-c3cc4cccc(-c5cccc6ccccc56)c4cc32)cc1]

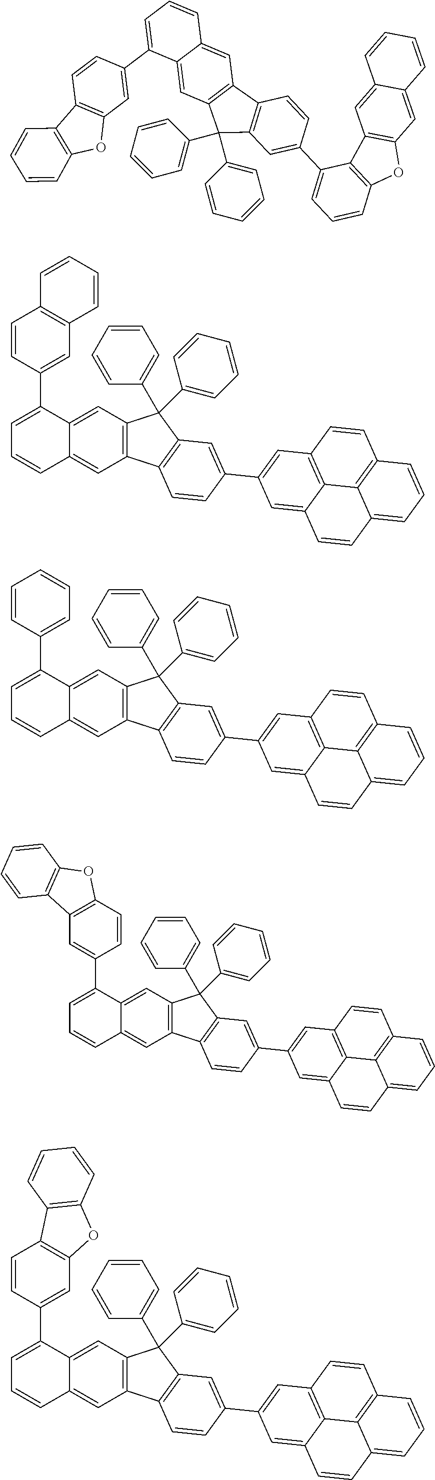

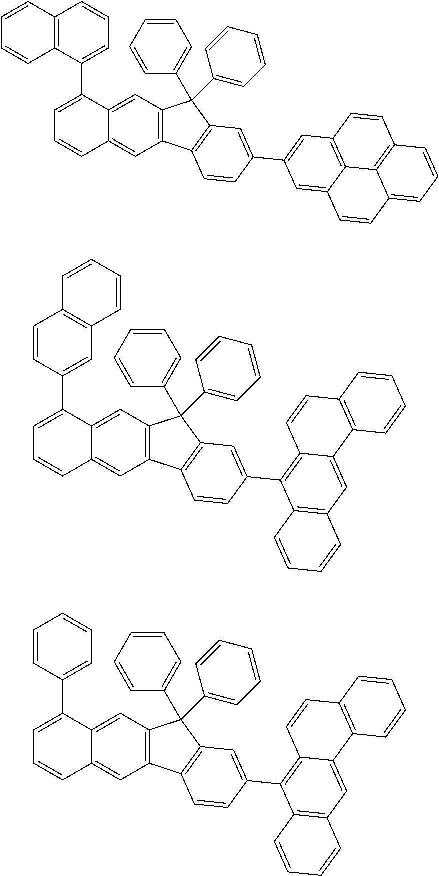

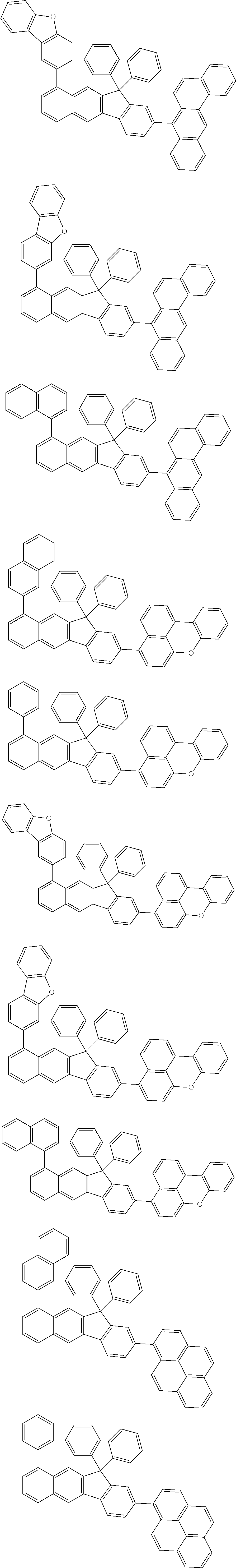

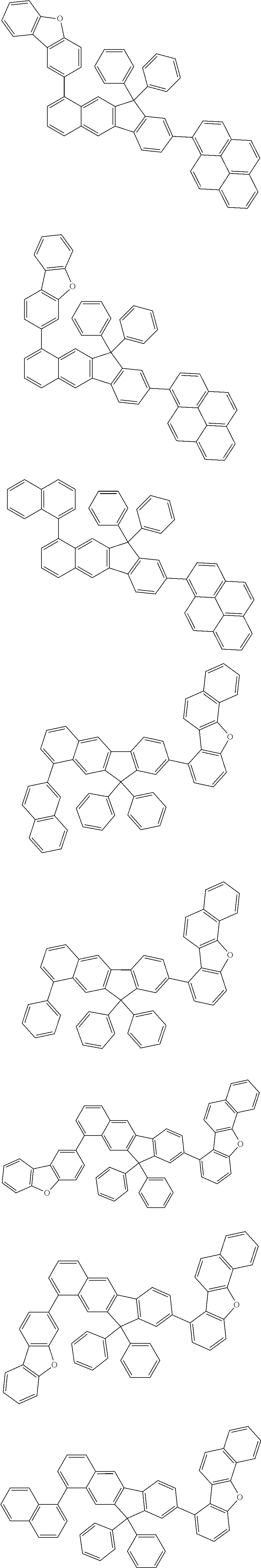

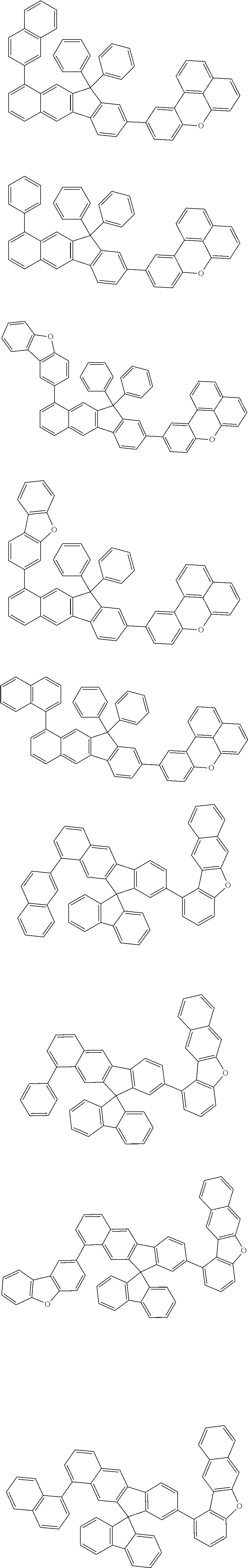

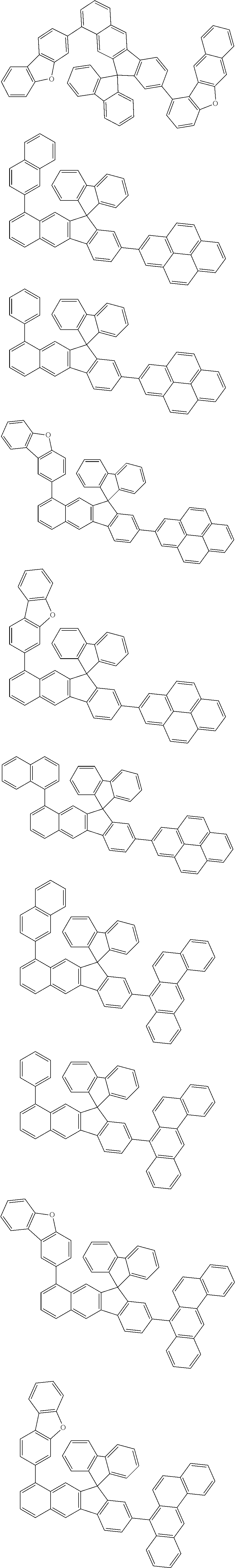

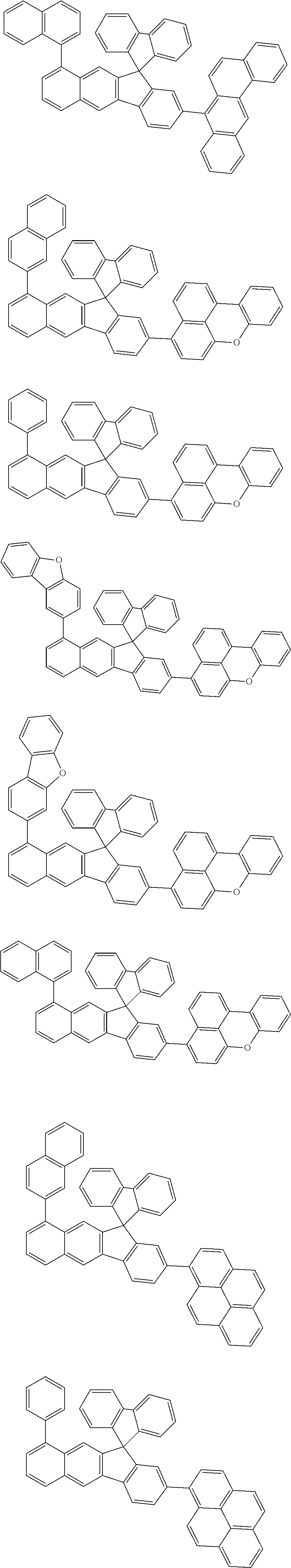

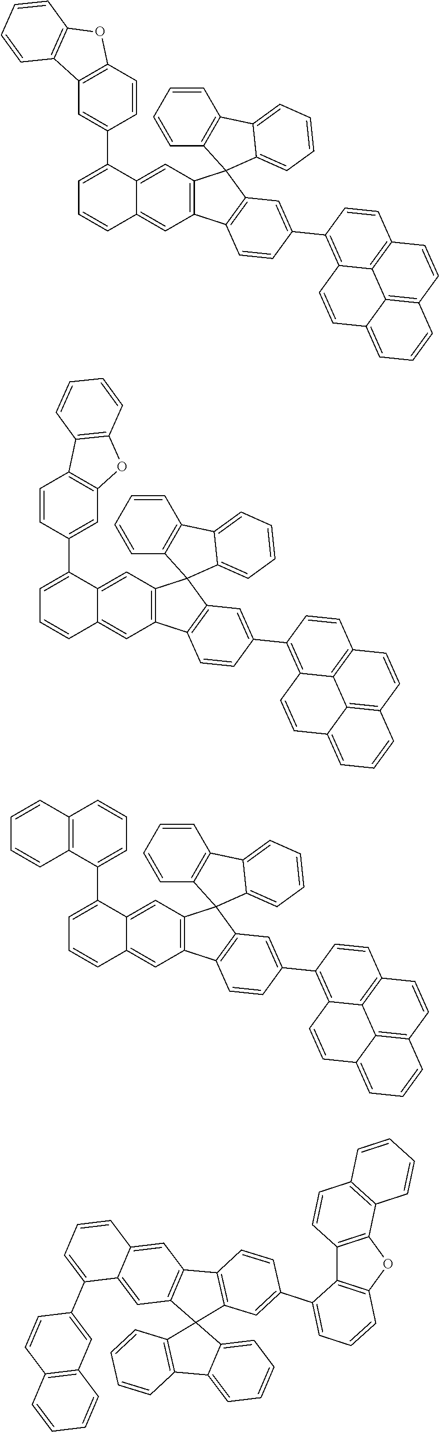

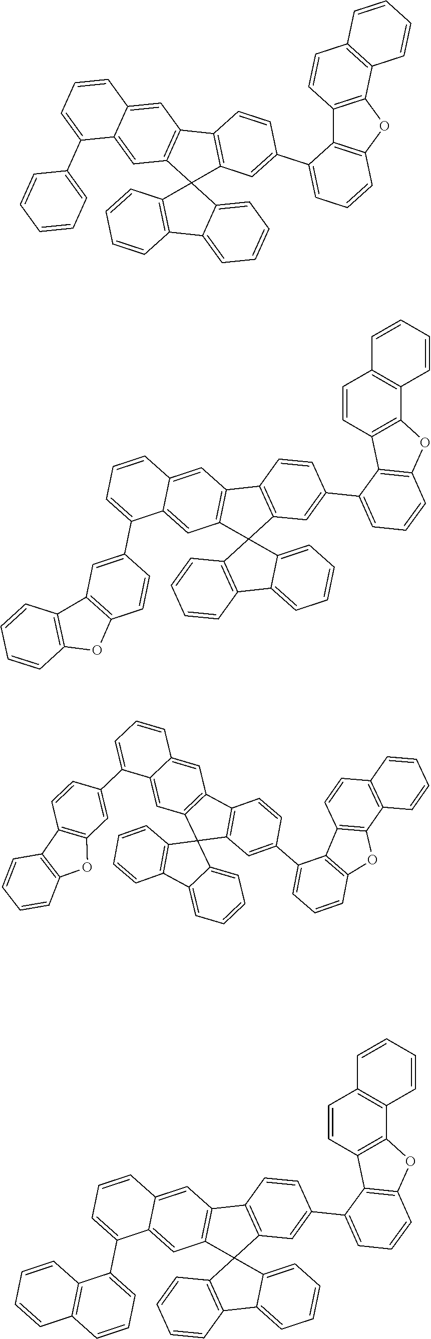 [SMILES: c1ccc(-c2cccc3cc4c(cc23)C2(c3ccccc3-c3ccccc32)c2cc(-c3cccc5oc6c7ccccc7ccc6c35)ccc2-4)cc1 ; c1ccc2c(c1)-c1ccccc1C21c2cc(-c3cccc4oc5c6ccccc6ccc5c34)ccc2-c2cc3cccc(-c4ccc5oc6ccccc6c5c4)c3cc21 ; c1ccc2c(c1)-c1ccccc1C21c2cc(-c3cccc4oc5c6ccccc6ccc5c34)ccc2-c2cc3cccc(-c4ccc5c(c4)oc4ccccc45)c3cc21 ; c1ccc2c(c1)-c1ccccc1C21c2cc(-c3cccc4oc5c6ccccc6ccc5c34)ccc2-c2cc3cccc(-c4cccc5ccccc45)c3cc21]

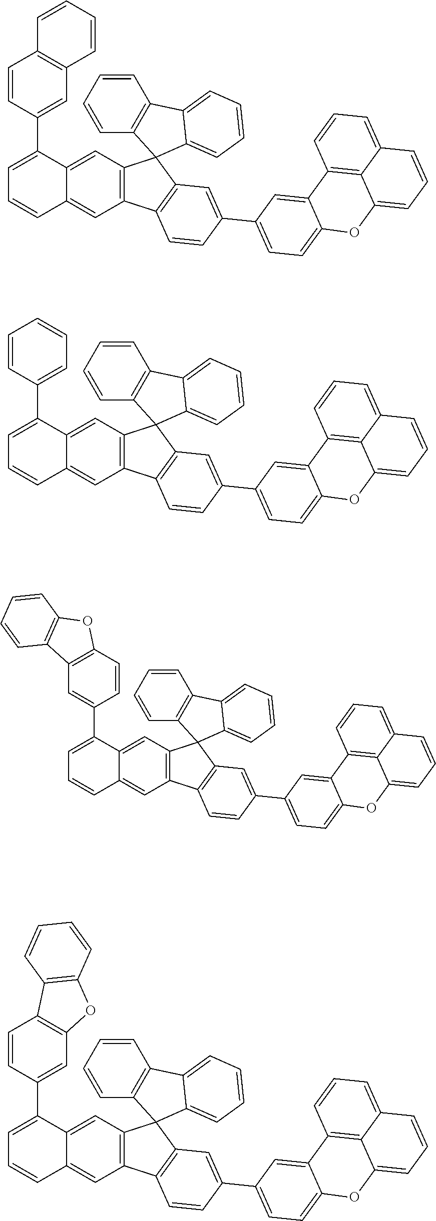 [SMILES: c1ccc2c(c1)-c1ccccc1C21c2cc(-c3ccc4c(c3)-c3cccc5cccc(c35)O4)ccc2-c2cc3cccc(-c4ccc5ccccc5c4)c3cc21 ; c1ccc(-c2cccc3cc4c(cc23)C2(c3ccccc3-c3ccccc32)c2cc(-c3ccc5c(c3)-c3cccc6cccc(c36)O5)ccc2-4)cc1 ; c1ccc2c(c1)-c1ccccc1C21c2cc(-c3ccc4c(c3)-c3cccc5cccc(c35)O4)ccc2-c2cc3cccc(-c4ccc5oc6ccccc6c5c4)c3cc21 ; c1ccc2c(c1)-c1ccccc1C21c2cc(-c3ccc4c(c3)-c3cccc5cccc(c35)O4)ccc2-c2cc3cccc(-c4ccc5c(c4)oc4ccccc45)c3cc21]

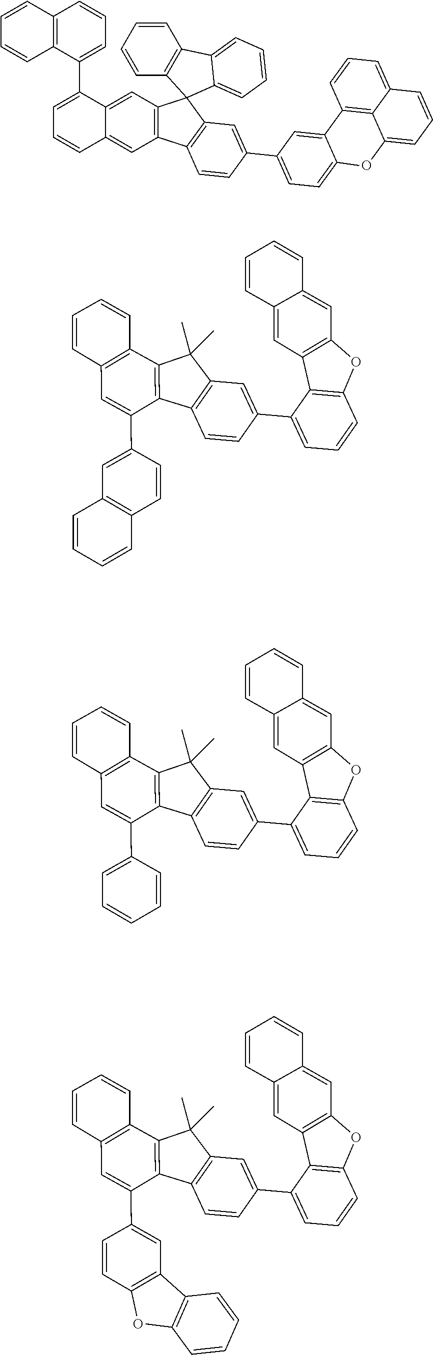 [SMILES: c1ccc2c(c1)-c1ccccc1C21c2cc(-c3ccc4c(c3)-c3cccc5cccc(c35)O4)ccc2-c2cc3cccc(-c4cccc5ccccc45)c3cc21 ; CC1(C)c2cc(-c3cccc4oc5cc6ccccc6cc5c34)ccc2-c2c(-c3ccc4ccccc4c3)cc3ccccc3c21 ; CC1(C)c2cc(-c3cccc4oc5cc6ccccc6cc5c34)ccc2-c2c(-c3ccccc3)cc3ccccc3c21 ; CC1(C)c2cc(-c3cccc4oc5cc6ccccc6cc5c34)ccc2-c2c(-c3ccc4oc5ccccc5c4c3)cc3ccccc3c21]

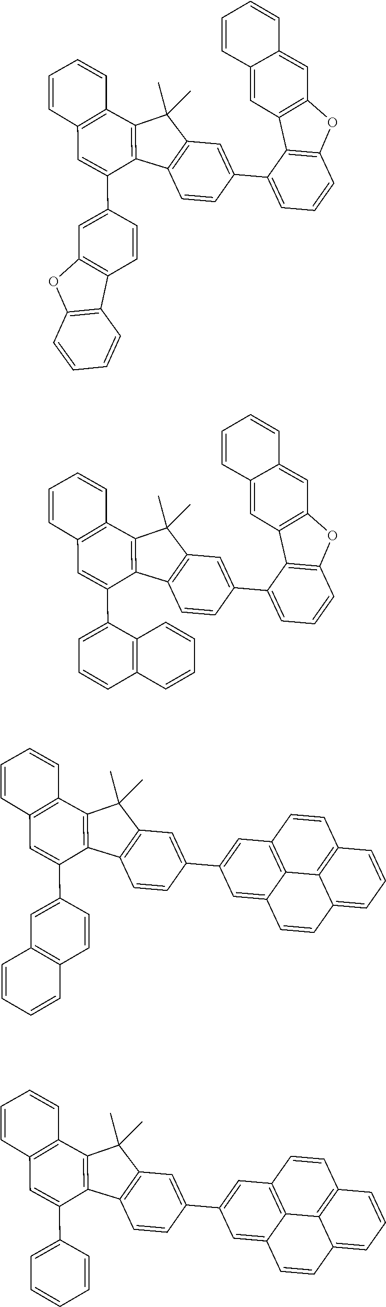

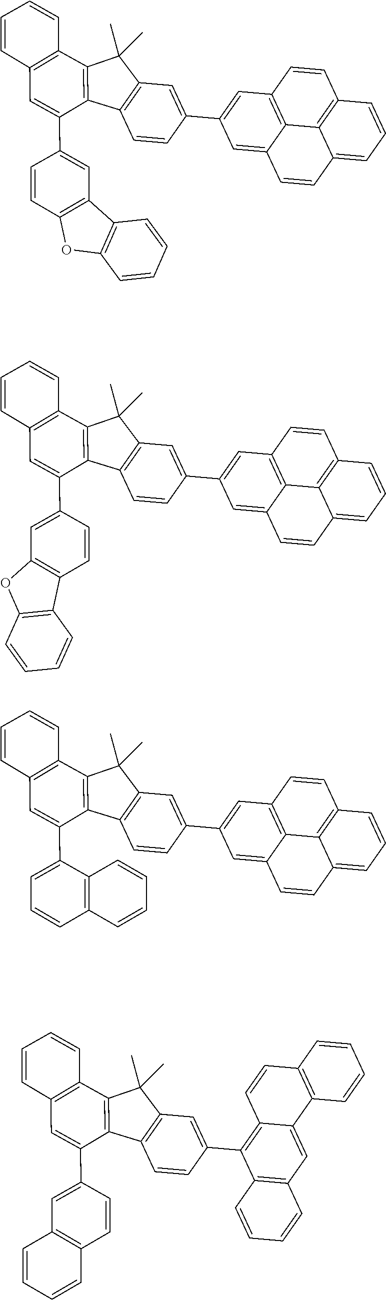

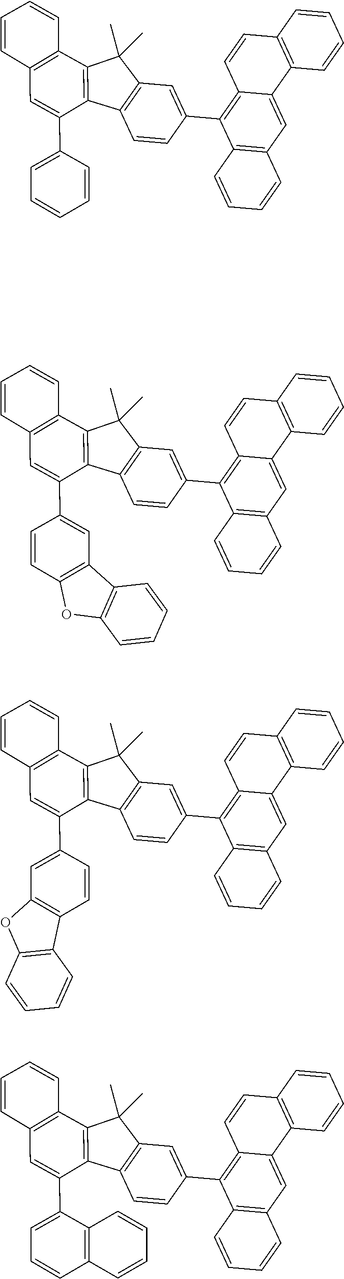

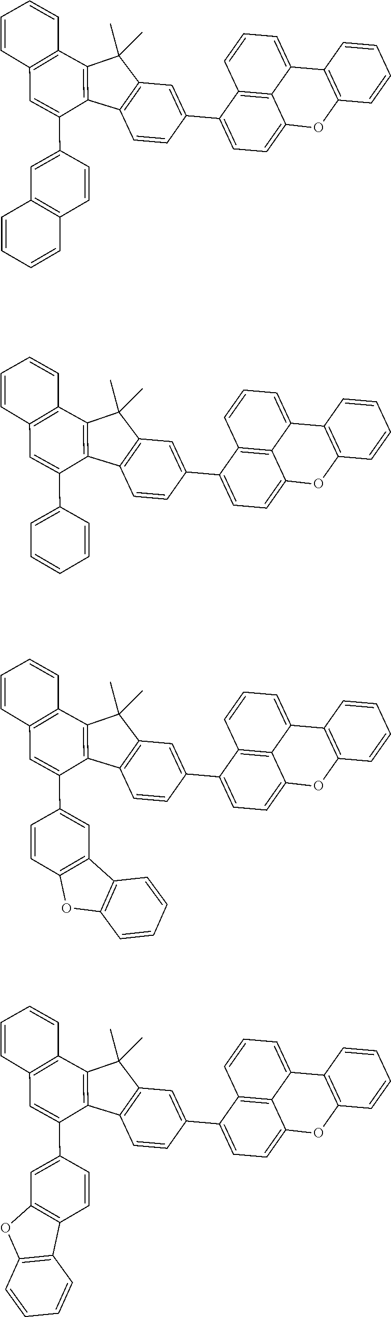 [SMILES: CC1(C)c2cc(-c3ccc4c5c(cccc35)-c3ccccc3O4)ccc2-c2c(-c3ccc4ccccc4c3)cc3ccccc3c21 ; CC1(C)c2cc(-c3ccc4c5c(cccc35)-c3ccccc3O4)ccc2-c2c(-c3ccccc3)cc3ccccc3c21 ; CC1(C)c2cc(-c3ccc4c5c(cccc35)-c3ccccc3O4)ccc2-c2c(-c3ccc4oc5ccccc5c4c3)cc3ccccc3c21 ; CC1(C)c2cc(-c3ccc4c5c(cccc35)-c3ccccc3O4)ccc2-c2c(-c3ccc4c(c3)oc3ccccc34)cc3ccccc3c21]

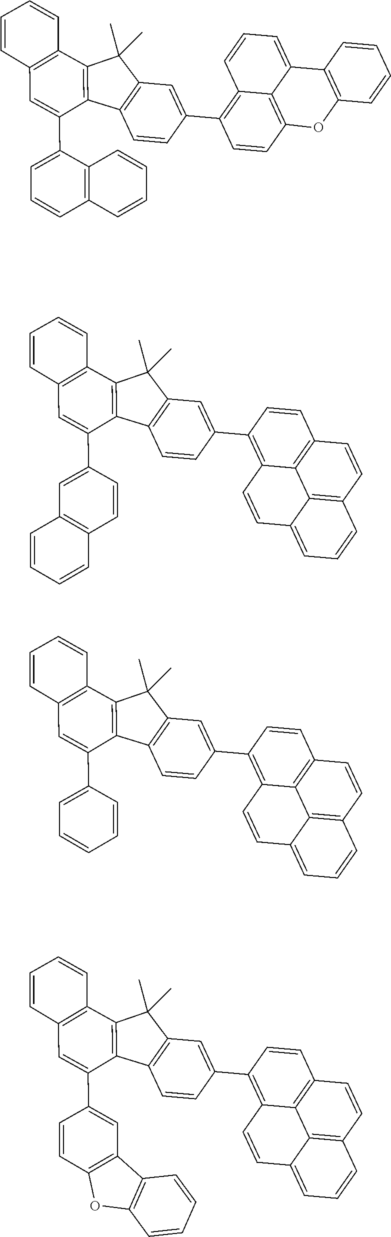

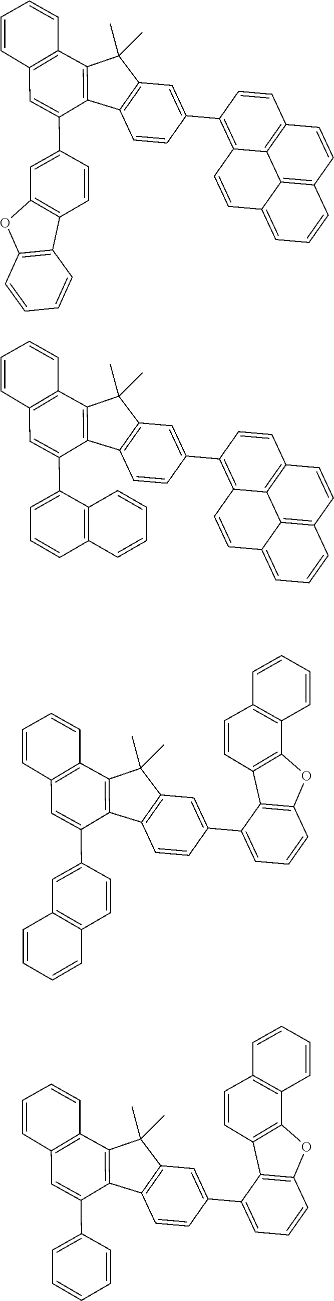

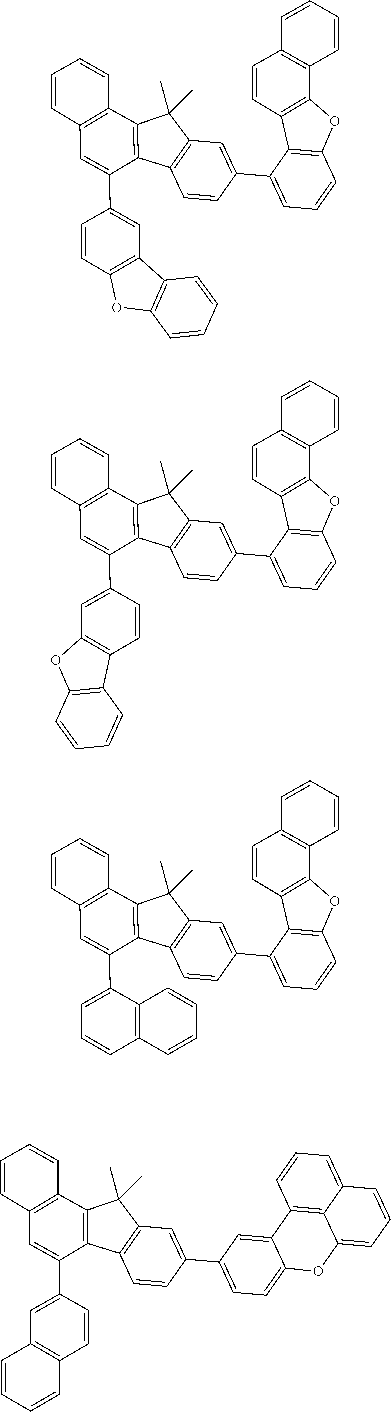 [SMILES: CC1(C)c2cc(-c3cccc4oc5c6ccccc6ccc5c34)ccc2-c2c(-c3ccc4oc5ccccc5c4c3)cc3ccccc3c21 ; CC1(C)c2cc(-c3cccc4oc5c6ccccc6ccc5c34)ccc2-c2c(-c3ccc4c(c3)oc3ccccc34)cc3ccccc3c21 ; CC1(C)c2cc(-c3cccc4oc5c6ccccc6ccc5c34)ccc2-c2c(-c3cccc4ccccc34)cc3ccccc3c21 ; CC1(C)c2cc(-c3ccc4c(c3)-c3cccc5cccc(c35)O4)ccc2-c2c(-c3ccc4ccccc4c3)cc3ccccc3c21]

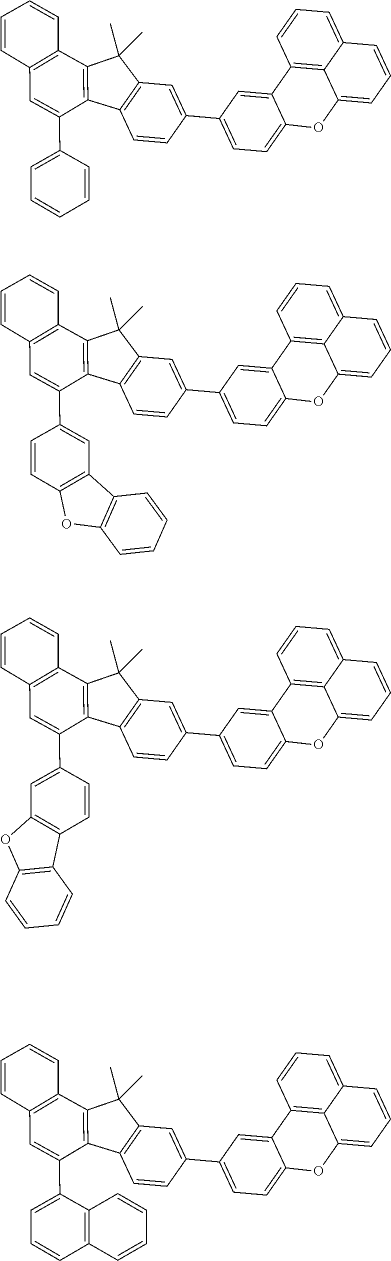 [SMILES: CC1(C)c2cc(-c3ccc4c(c3)-c3cccc5cccc(c35)O4)ccc2-c2c(-c3ccccc3)cc3ccccc3c21 ; CC1(C)c2cc(-c3ccc4c(c3)-c3cccc5cccc(c35)O4)ccc2-c2c(-c3ccc4oc5ccccc5c4c3)cc3ccccc3c21 ; CC1(C)c2cc(-c3ccc4c(c3)-c3cccc5cccc(c35)O4)ccc2-c2c(-c3ccc4c(c3)oc3ccccc34)cc3ccccc3c21 ; CC1(C)c2cc(-c3ccc4c(c3)-c3cccc5cccc(c35)O4)ccc2-c2c(-c3cccc4ccccc34)cc3ccccc3c21]

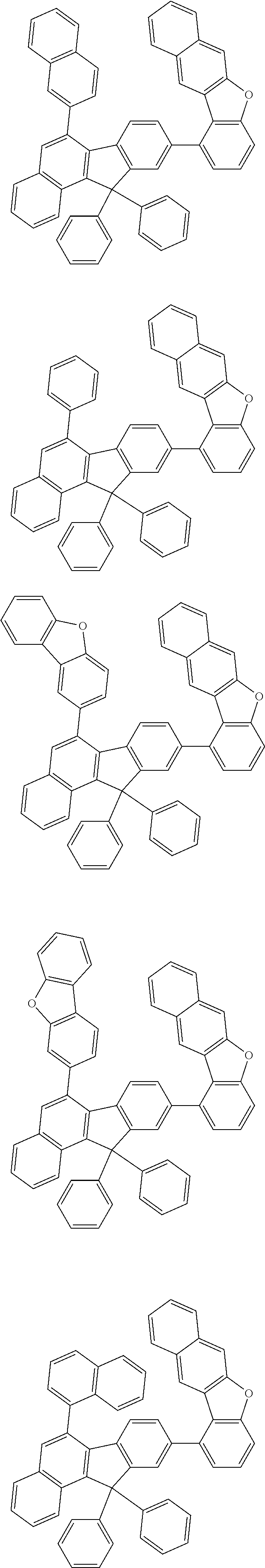 [SMILES: c1ccc(C2(c3ccccc3)c3cc(-c4cccc5oc6cc7ccccc7cc6c45)ccc3-c3c(-c4ccc5ccccc5c4)cc4ccccc4c32)cc1 ; c1ccc(-c2cc3ccccc3c3c2-c2ccc(-c4cccc5oc6cc7ccccc7cc6c45)cc2C3(c2ccccc2)c2ccccc2)cc1 ; c1ccc(C2(c3ccccc3)c3cc(-c4cccc5oc6cc7ccccc7cc6c45)ccc3-c3c(-c4ccc5oc6ccccc6c5c4)cc4ccccc4c32)cc1 ; c1ccc(C2(c3ccccc3)c3cc(-c4cccc5oc6cc7ccccc7cc6c45)ccc3-c3c(-c4ccc5c(c4)oc4ccccc45)cc4ccccc4c32)cc1 ; c1ccc(C2(c3ccccc3)c3cc(-c4cccc5oc6cc7ccccc7cc6c45)ccc3-c3c(-c4cccc5ccccc45)cc4ccccc4c32)cc1]

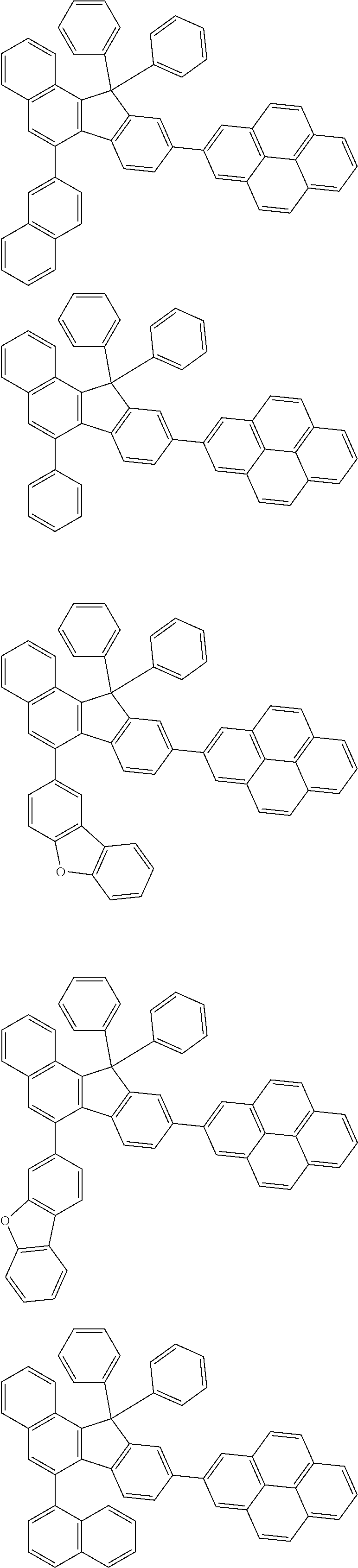

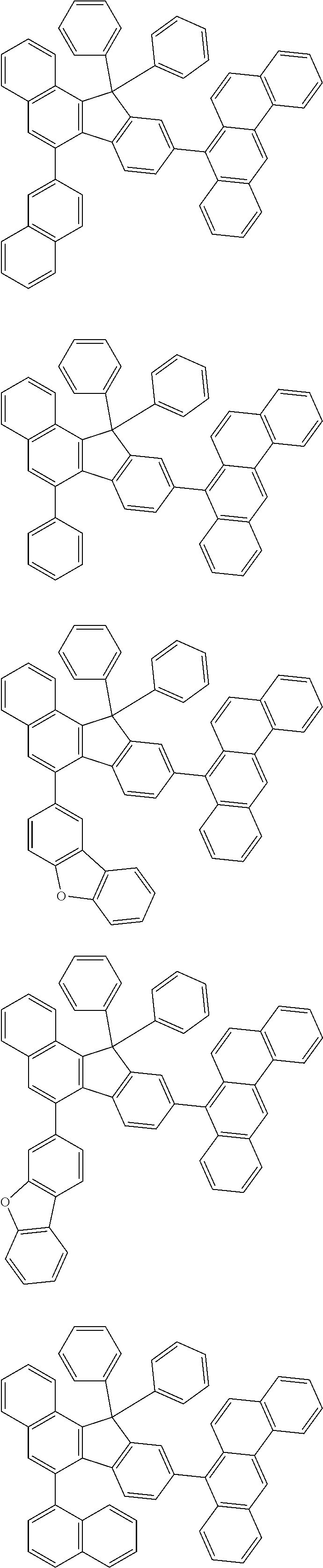

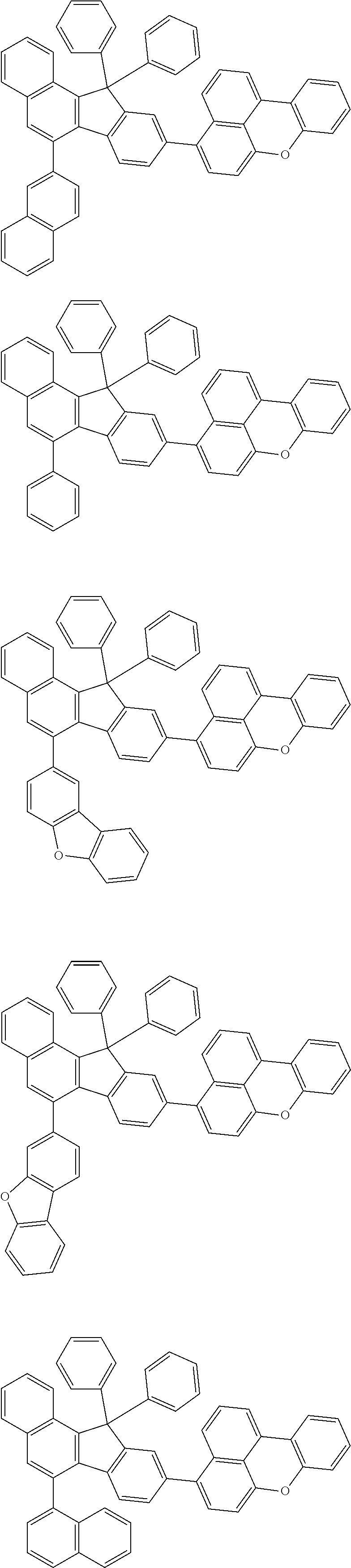 [SMILES: c1ccc(C2(c3ccccc3)c3cc(-c4ccc5c6c(cccc46)-c4ccccc4O5)ccc3-c3c(-c4ccc5ccccc5c4)cc4ccccc4c32)cc1 ; c1ccc(-c2cc3ccccc3c3c2-c2ccc(-c4ccc5c6c(cccc46)-c4ccccc4O5)cc2C3(c2ccccc2)c2ccccc2)cc1 ; c1ccc(C2(c3ccccc3)c3cc(-c4ccc5c6c(cccc46)-c4ccccc4O5)ccc3-c3c(-c4ccc5oc6ccccc6c5c4)cc4ccccc4c32)cc1 ; c1ccc(C2(c3ccccc3)c3cc(-c4ccc5c6c(cccc46)-c4ccccc4O5)ccc3-c3c(-c4ccc5c(c4)oc4ccccc45)cc4ccccc4c32)cc1 ; c1ccc(C2(c3ccccc3)c3cc(-c4ccc5c6c(cccc46)-c4ccccc4O5)ccc3-c3c(-c4cccc5ccccc45)cc4ccccc4c32)cc1]

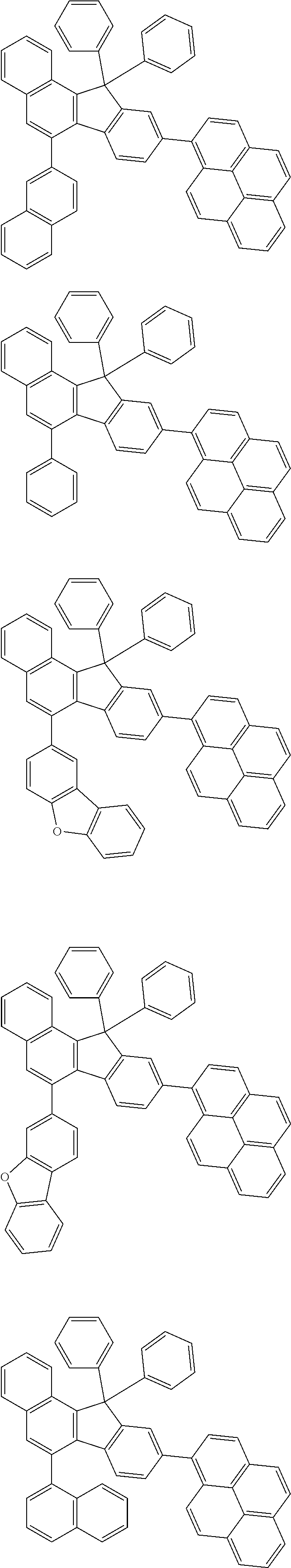

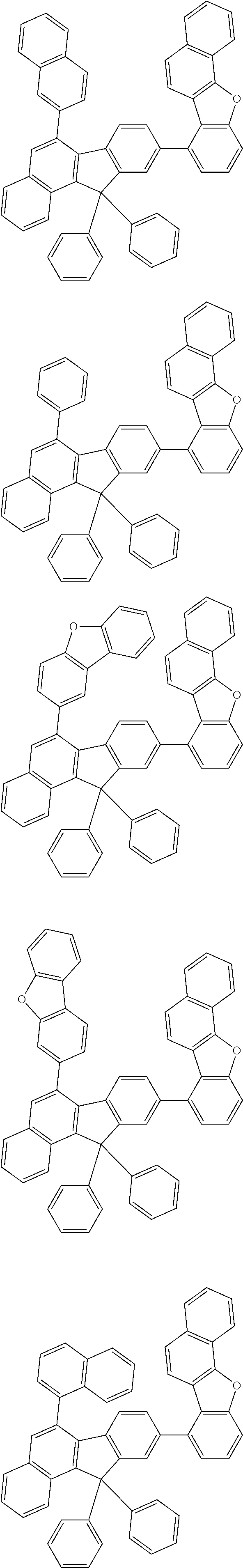 [SMILES: c1ccc(C2(c3ccccc3)c3cc(-c4cccc5oc6c7ccccc7ccc6c45)ccc3-c3c(-c4ccc5ccccc5c4)cc4ccccc4c32)cc1 ; c1ccc(-c2cc3ccccc3c3c2-c2ccc(-c4cccc5oc6c7ccccc7ccc6c45)cc2C3(c2ccccc2)c2ccccc2)cc1 ; c1ccc(C2(c3ccccc3)c3cc(-c4cccc5oc6c7ccccc7ccc6c45)ccc3-c3c(-c4ccc5oc6ccccc6c5c4)cc4ccccc4c32)cc1 ; c1ccc(C2(c3ccccc3)c3cc(-c4cccc5oc6c7ccccc7ccc6c45)ccc3-c3c(-c4ccc5c(c4)oc4ccccc45)cc4ccccc4c32)cc1 ; c1ccc(C2(c3ccccc3)c3cc(-c4cccc5oc6c7ccccc7ccc6c45)ccc3-c3c(-c4cccc5ccccc45)cc4ccccc4c32)cc1]

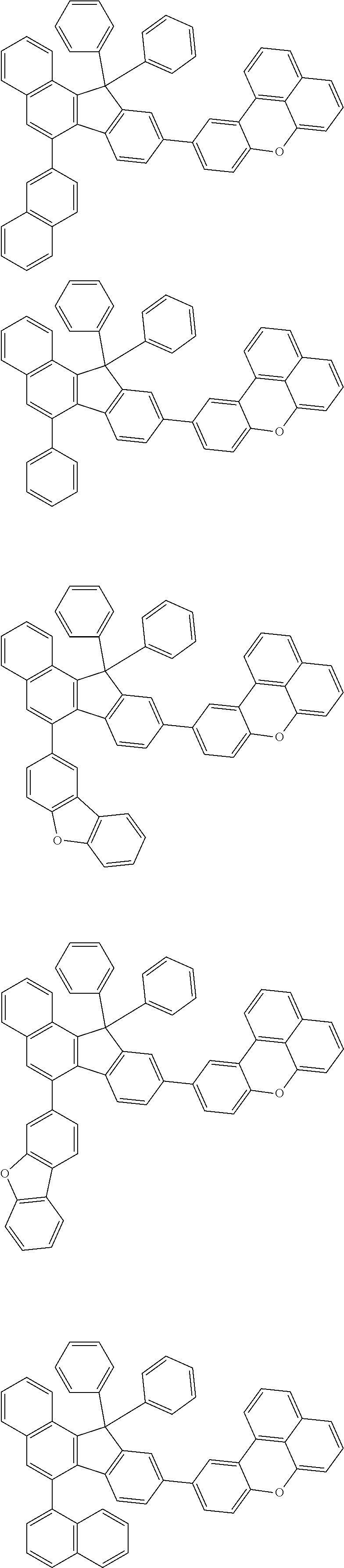

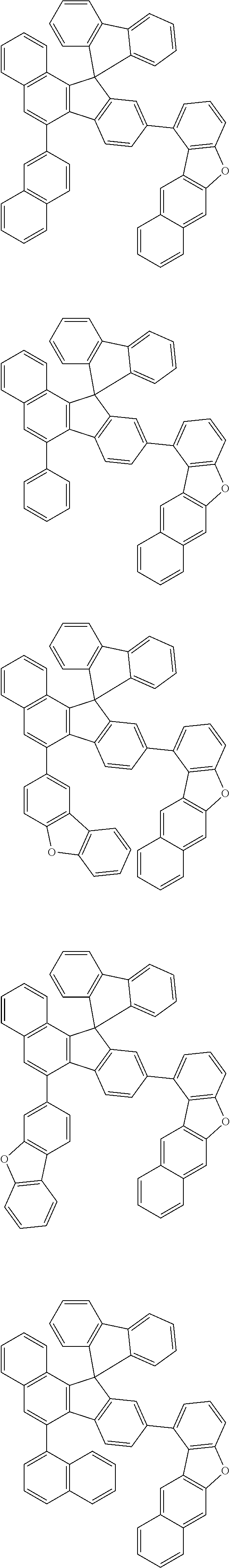 [SMILES: c1ccc2c(c1)-c1ccccc1C21c2cc(-c3cccc4oc5cc6ccccc6cc5c34)ccc2-c2c(-c3ccc4ccccc4c3)cc3ccccc3c21 ; c1ccc(-c2cc3ccccc3c3c2-c2ccc(-c4cccc5oc6cc7ccccc7cc6c45)cc2C32c3ccccc3-c3ccccc32)cc1 ; c1ccc2c(c1)-c1ccccc1C21c2cc(-c3cccc4oc5cc6ccccc6cc5c34)ccc2-c2c(-c3ccc4oc5ccccc5c4c3)cc3ccccc3c21 ; c1ccc2c(c1)-c1ccccc1C21c2cc(-c3cccc4oc5cc6ccccc6cc5c34)ccc2-c2c(-c3ccc4c(c3)oc3ccccc34)cc3ccccc3c21 ; c1ccc2c(c1)-c1ccccc1C21c2cc(-c3cccc4oc5cc6ccccc6cc5c34)ccc2-c2c(-c3cccc4ccccc34)cc3ccccc3c21]

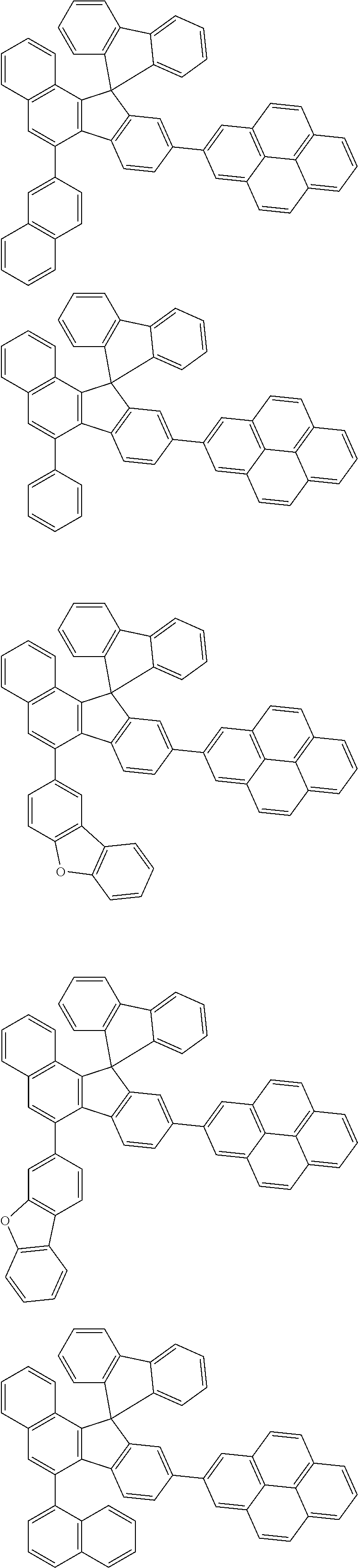

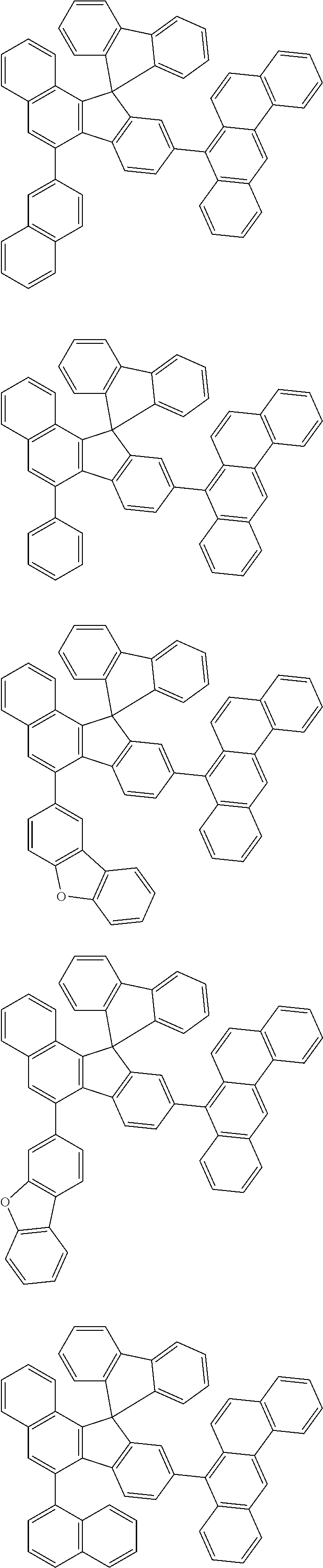

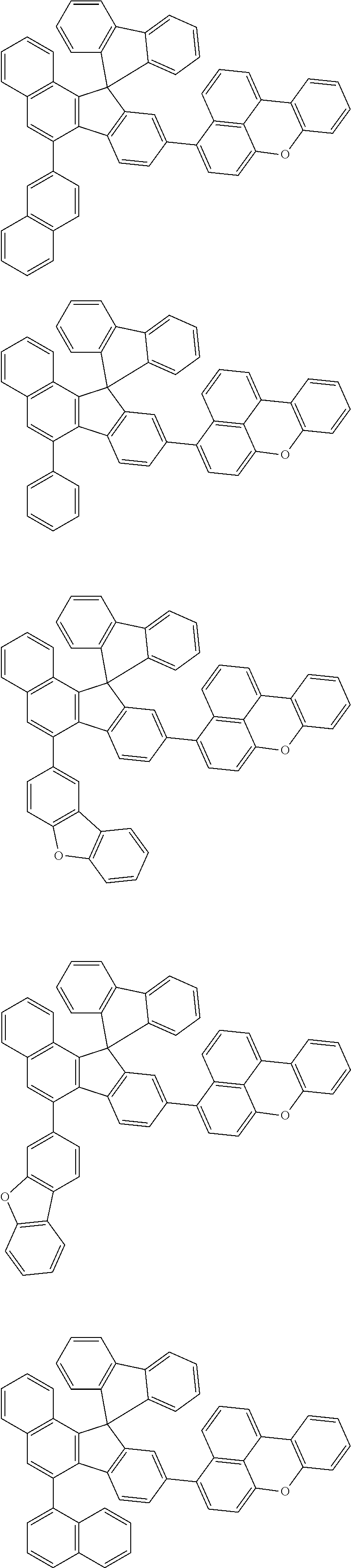 [SMILES: c1ccc2c(c1)Oc1ccc(-c3ccc4c(c3)C3(c5ccccc5-c5ccccc53)c3c-4c(-c4ccc5ccccc5c4)cc4ccccc34)c3cccc-2c13 ; c1ccc(-c2cc3ccccc3c3c2-c2ccc(-c4ccc5c6c(cccc46)-c4ccccc4O5)cc2C32c3ccccc3-c3ccccc32)cc1 ; c1ccc2c(c1)Oc1ccc(-c3ccc4c(c3)C3(c5ccccc5-c5ccccc53)c3c-4c(-c4ccc5oc6ccccc6c5c4)cc4ccccc34)c3cccc-2c13 ; c1ccc2c(c1)Oc1ccc(-c3ccc4c(c3)C3(c5ccccc5-c5ccccc53)c3c-4c(-c4ccc5c(c4)oc4ccccc45)cc4ccccc34)c3cccc-2c13 ; c1ccc2c(c1)Oc1ccc(-c3ccc4c(c3)C3(c5ccccc5-c5ccccc53)c3c-4c(-c4cccc5ccccc45)cc4ccccc34)c3cccc-2c13]

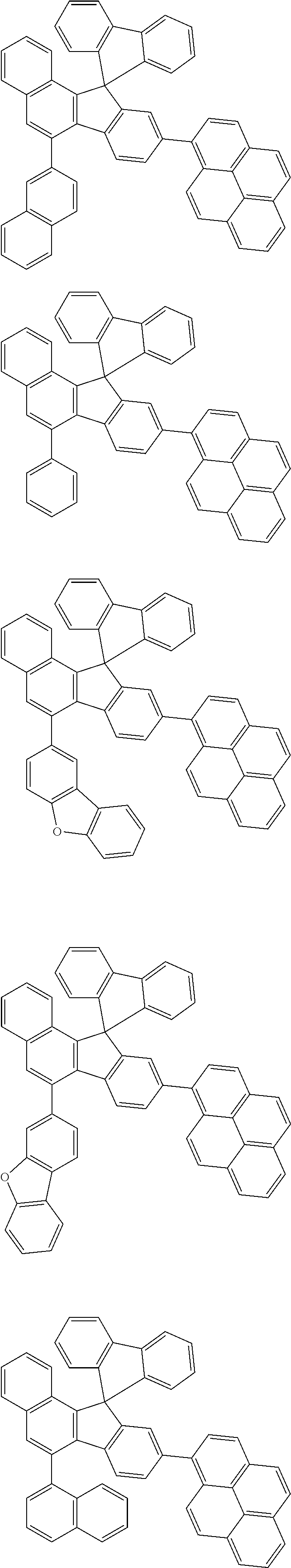

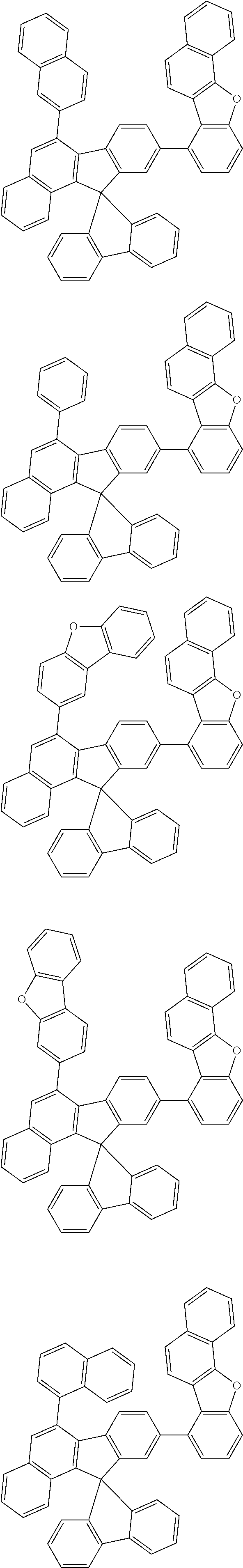 [SMILES: c1ccc2c(c1)-c1ccccc1C21c2cc(-c3cccc4oc5c6ccccc6ccc5c34)ccc2-c2c(-c3ccc4ccccc4c3)cc3ccccc3c21 ; c1ccc(-c2cc3ccccc3c3c2-c2ccc(-c4cccc5oc6c7ccccc7ccc6c45)cc2C32c3ccccc3-c3ccccc32)cc1 ; c1ccc2c(c1)-c1ccccc1C21c2cc(-c3cccc4oc5c6ccccc6ccc5c34)ccc2-c2c(-c3ccc4oc5ccccc5c4c3)cc3ccccc3c21 ; c1ccc2c(c1)-c1ccccc1C21c2cc(-c3cccc4oc5c6ccccc6ccc5c34)ccc2-c2c(-c3ccc4c(c3)oc3ccccc34)cc3ccccc3c21 ; c1ccc2c(c1)-c1ccccc1C21c2cc(-c3cccc4oc5c6ccccc6ccc5c34)ccc2-c2c(-c3cccc4ccccc34)cc3ccccc3c21]

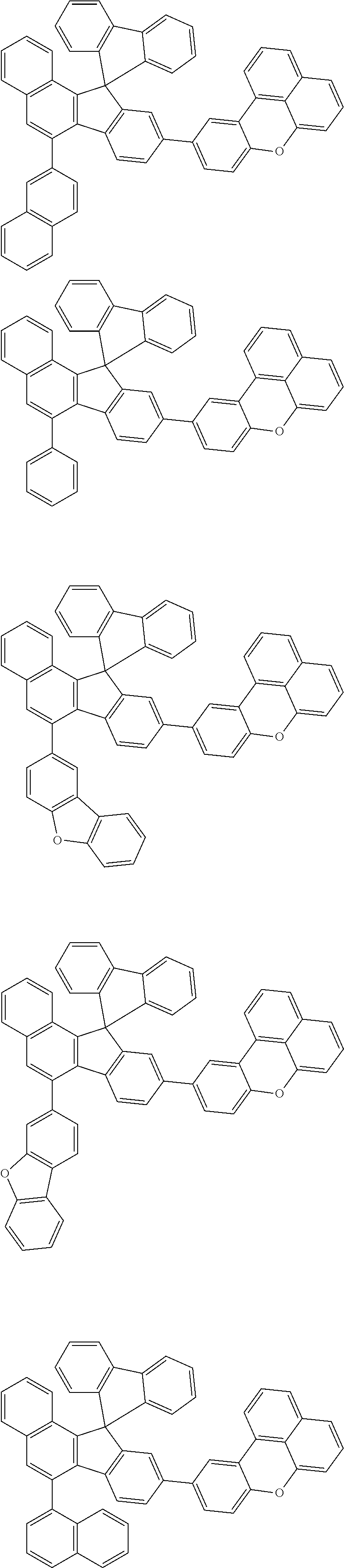

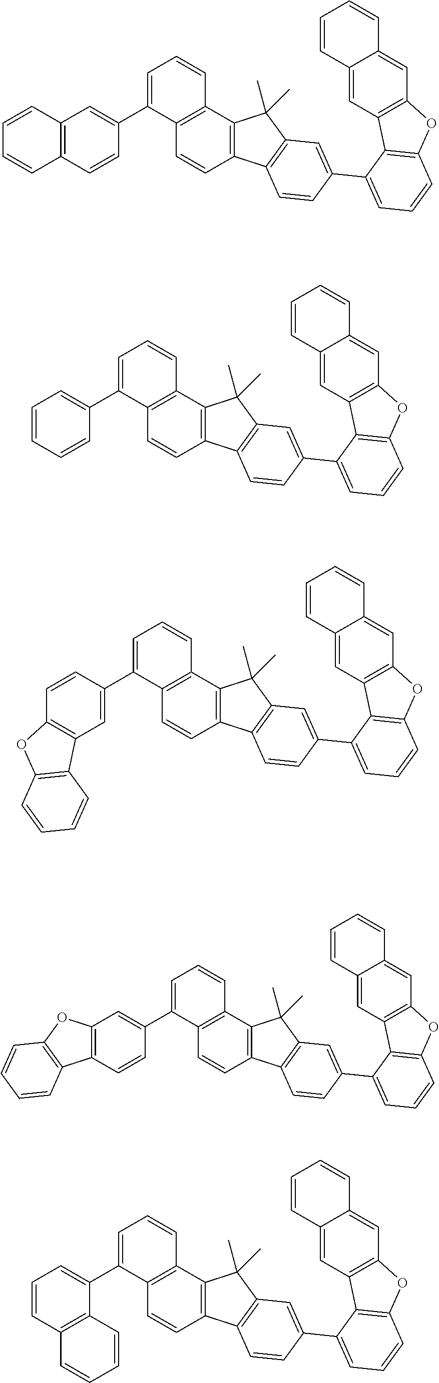 [SMILES: CC1(C)c2cc(-c3cccc4oc5cc6ccccc6cc5c34)ccc2-c2ccc3c(-c4ccc5ccccc5c4)cccc3c21 ; CC1(C)c2cc(-c3cccc4oc5cc6ccccc6cc5c34)ccc2-c2ccc3c(-c4ccccc4)cccc3c21 ; CC1(C)c2cc(-c3cccc4oc5cc6ccccc6cc5c34)ccc2-c2ccc3c(-c4ccc5oc6ccccc6c5c4)cccc3c21 ; CC1(C)c2cc(-c3cccc4oc5cc6ccccc6cc5c34)ccc2-c2ccc3c(-c4ccc5c(c4)oc4ccccc45)cccc3c21 ; CC1(C)c2cc(-c3cccc4oc5cc6ccccc6cc5c34)ccc2-c2ccc3c(-c4cccc5ccccc45)cccc3c21]

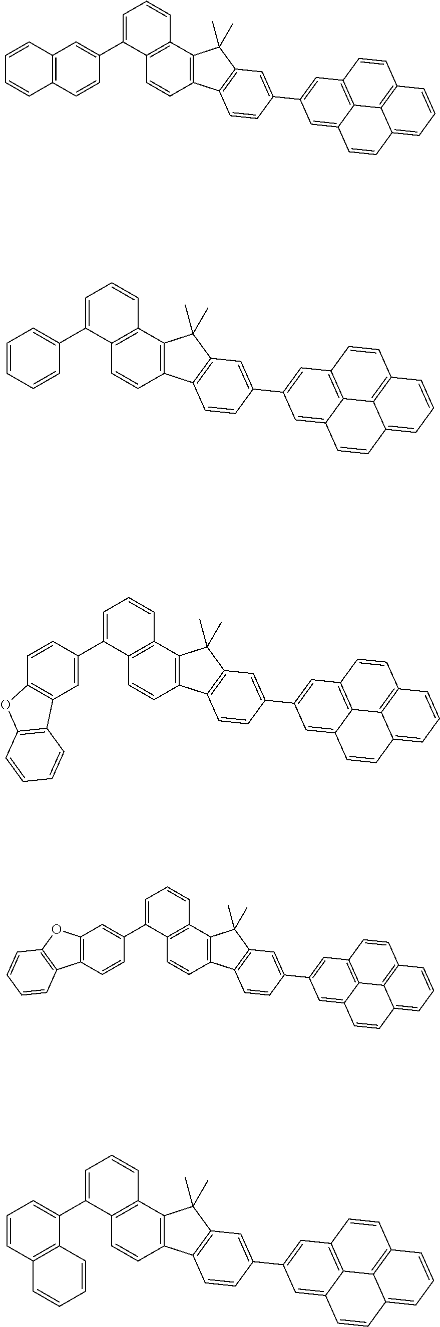

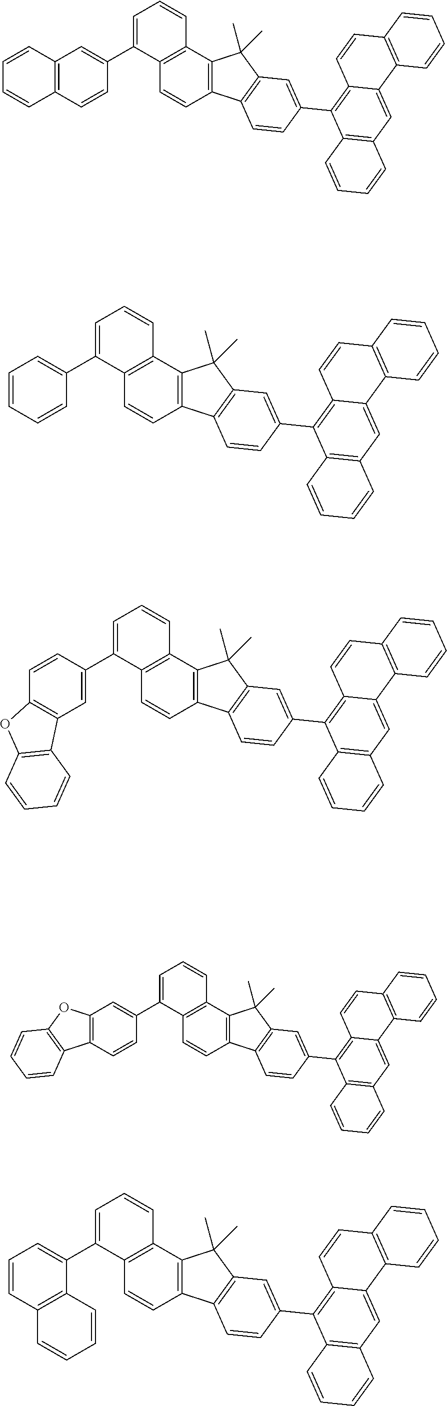

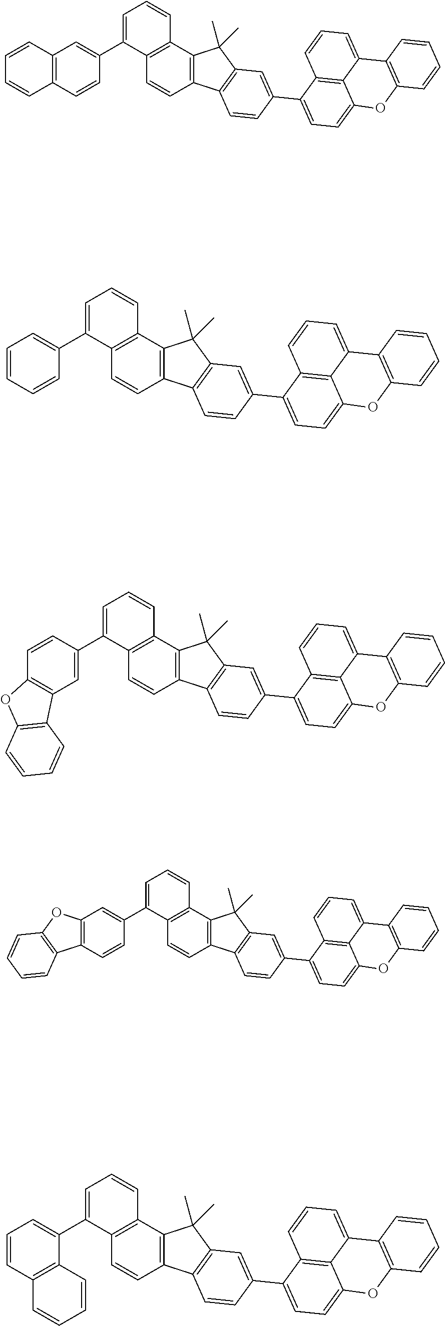 [SMILES: CC1(C)c2cc(-c3ccc4c5c(cccc35)-c3ccccc3O4)ccc2-c2ccc3c(-c4ccc5ccccc5c4)cccc3c21 ; CC1(C)c2cc(-c3ccc4c5c(cccc35)-c3ccccc3O4)ccc2-c2ccc3c(-c4ccccc4)cccc3c21 ; CC1(C)c2cc(-c3ccc4c5c(cccc35)-c3ccccc3O4)ccc2-c2ccc3c(-c4ccc5oc6ccccc6c5c4)cccc3c21 ; CC1(C)c2cc(-c3ccc4c5c(cccc35)-c3ccccc3O4)ccc2-c2ccc3c(-c4ccc5c(c4)oc4ccccc45)cccc3c21 ; CC1(C)c2cc(-c3ccc4c5c(cccc35)-c3ccccc3O4)ccc2-c2ccc3c(-c4cccc5ccccc45)cccc3c21]

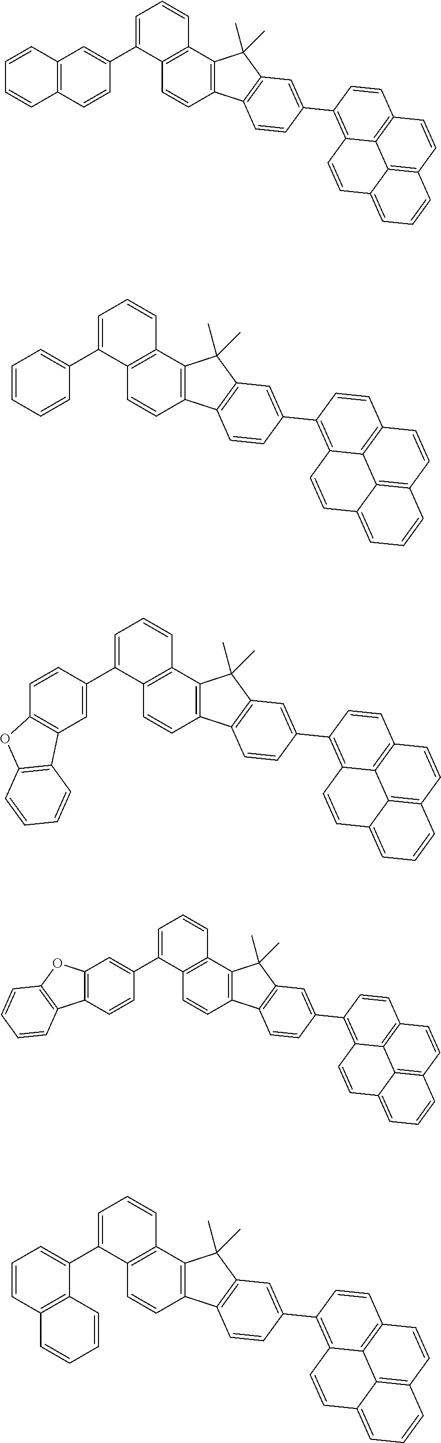

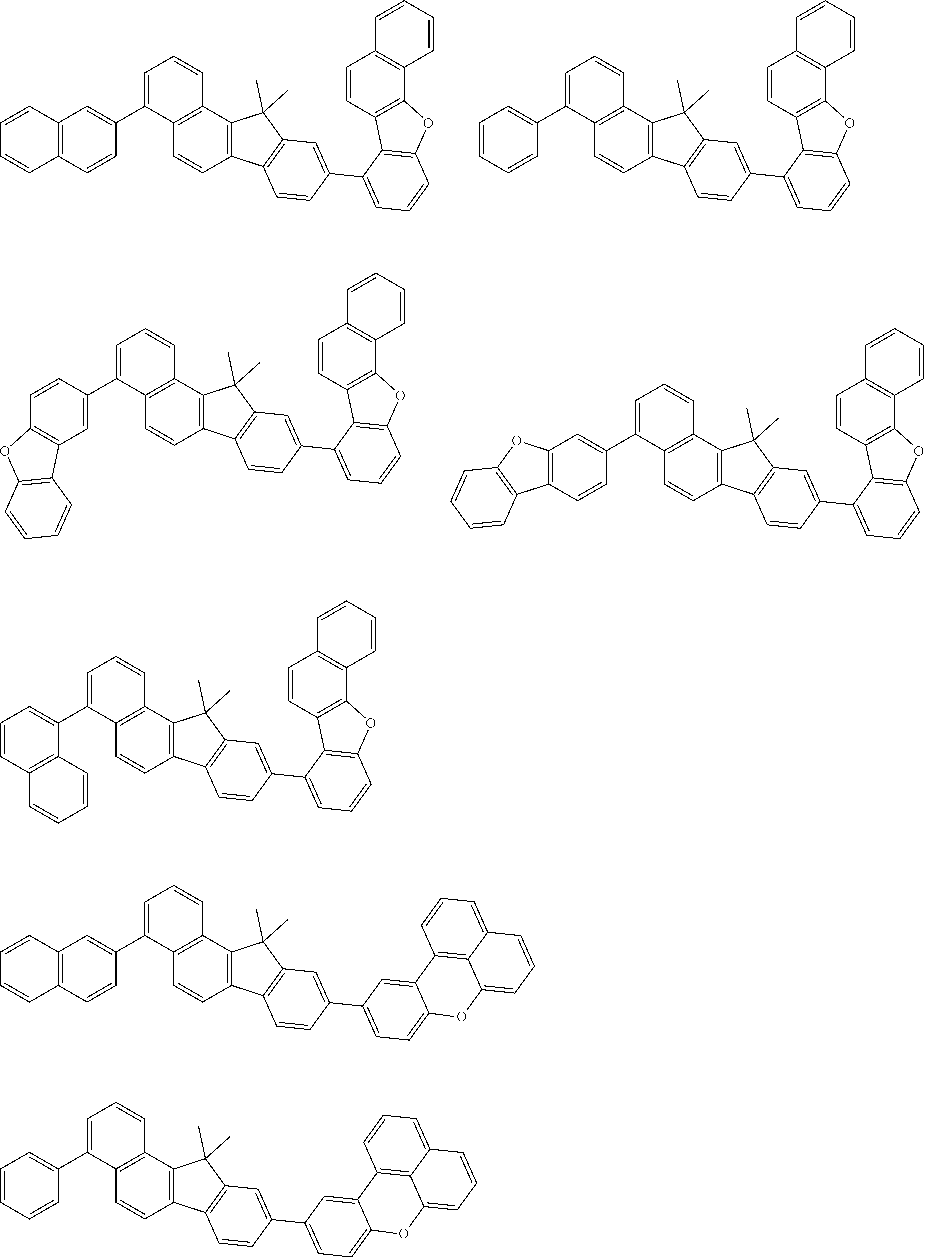

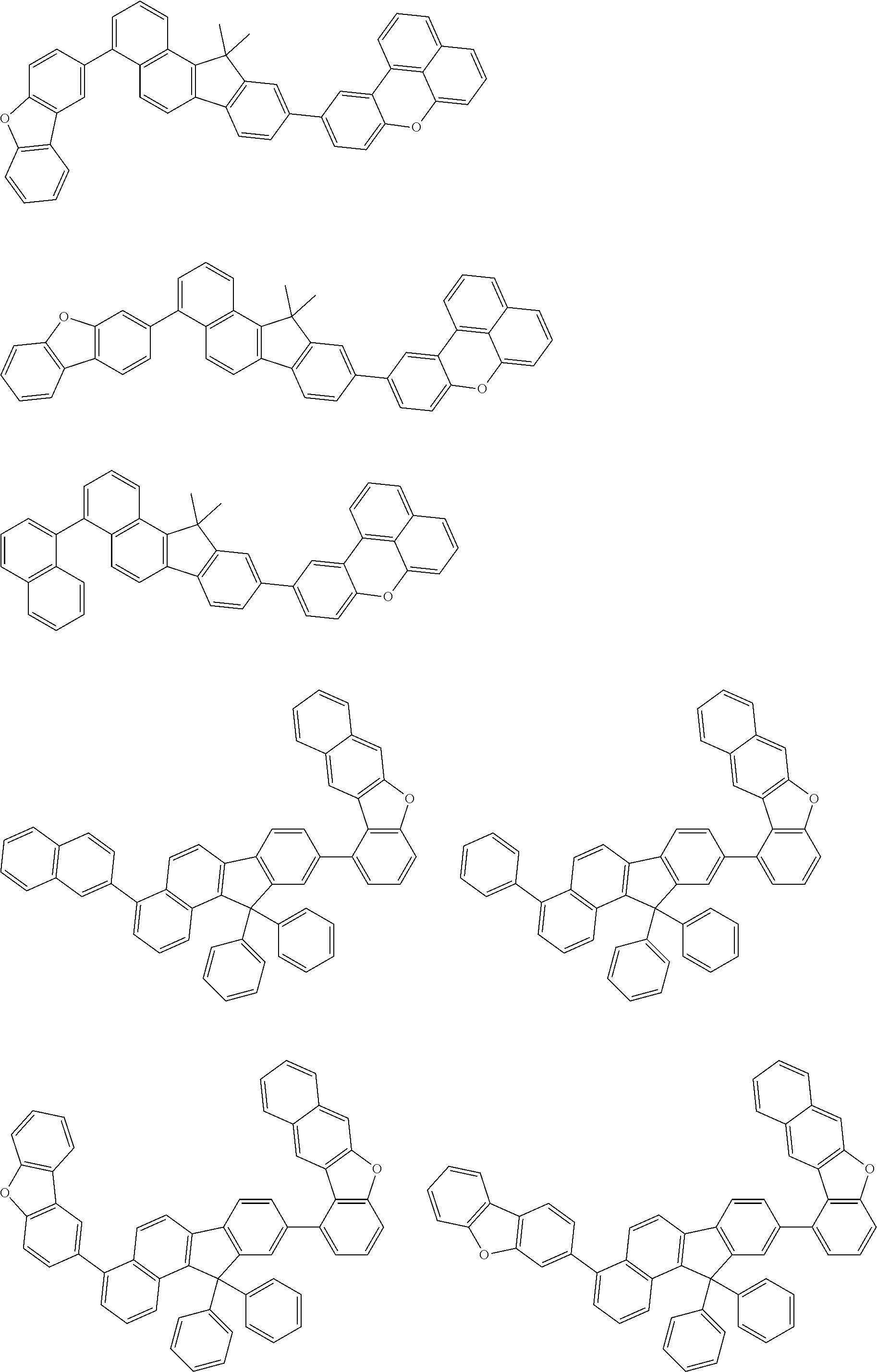

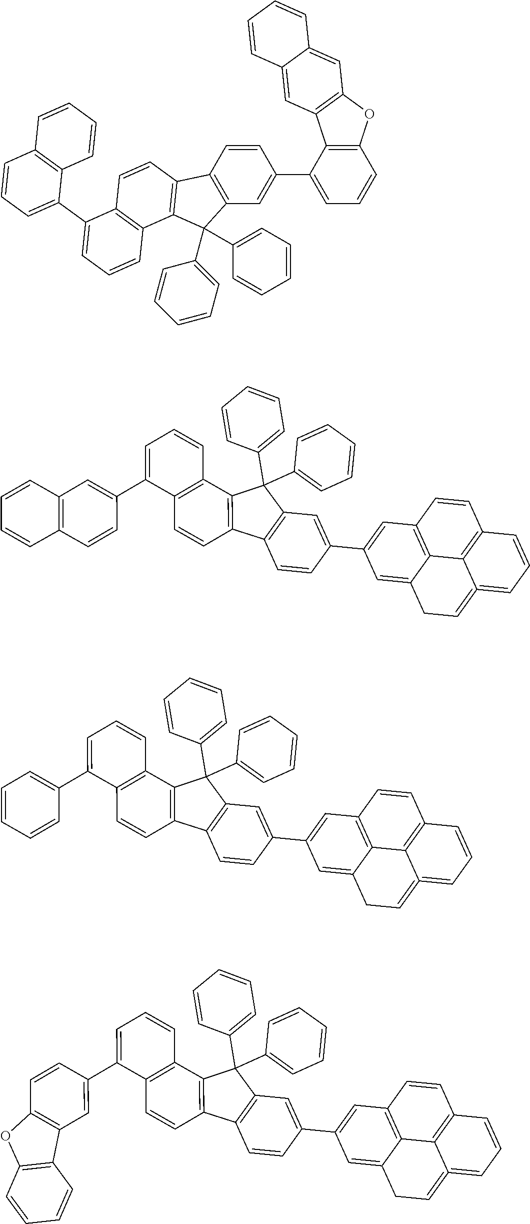

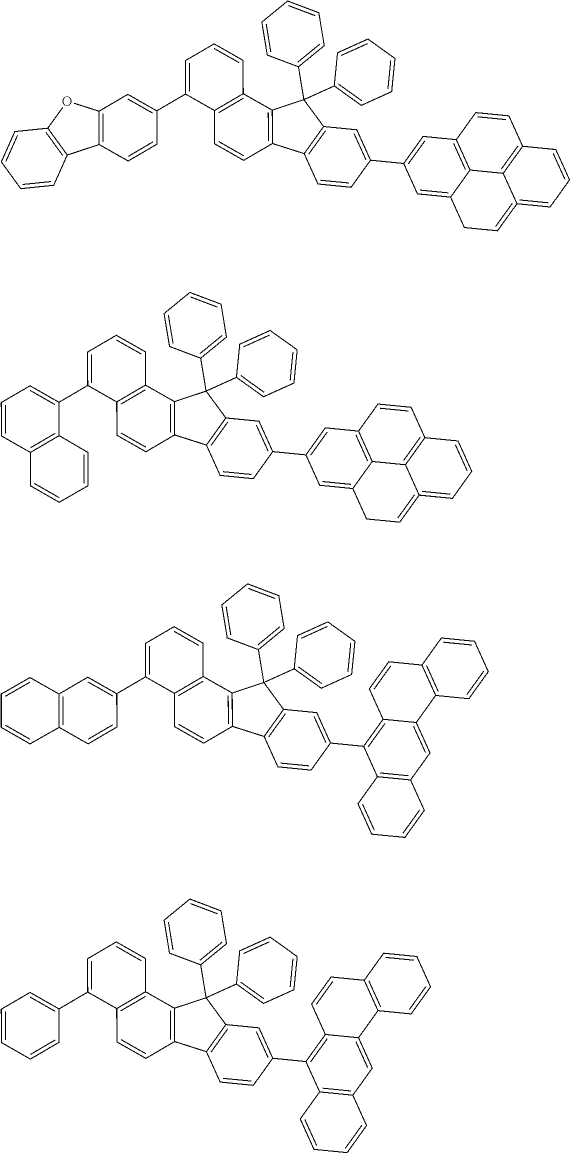

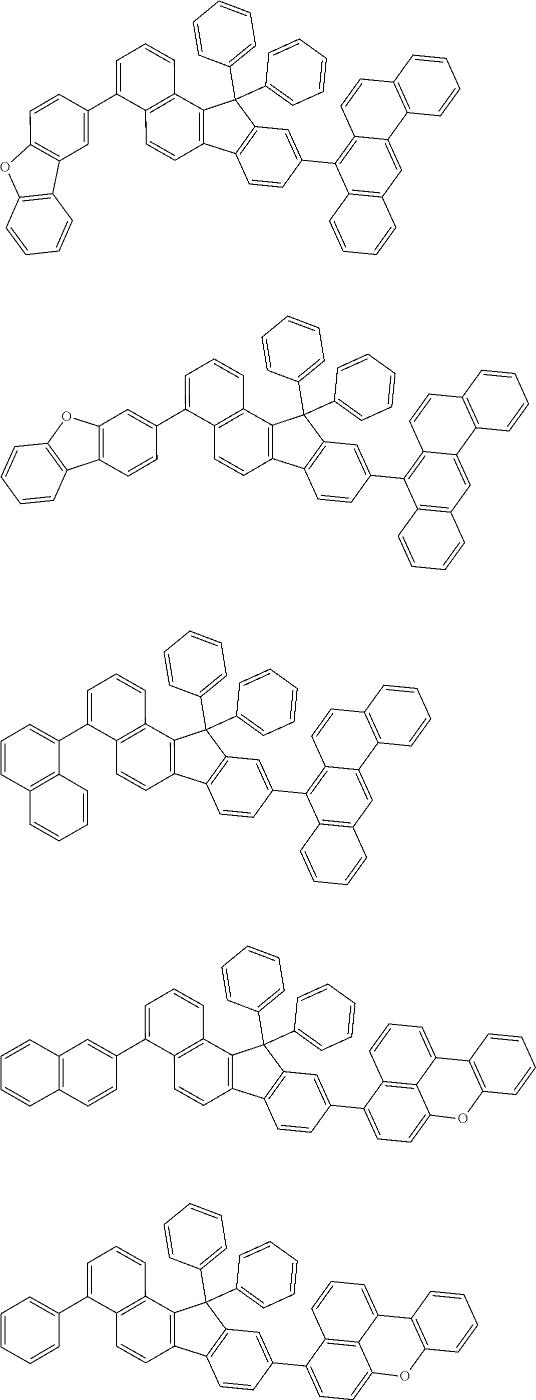

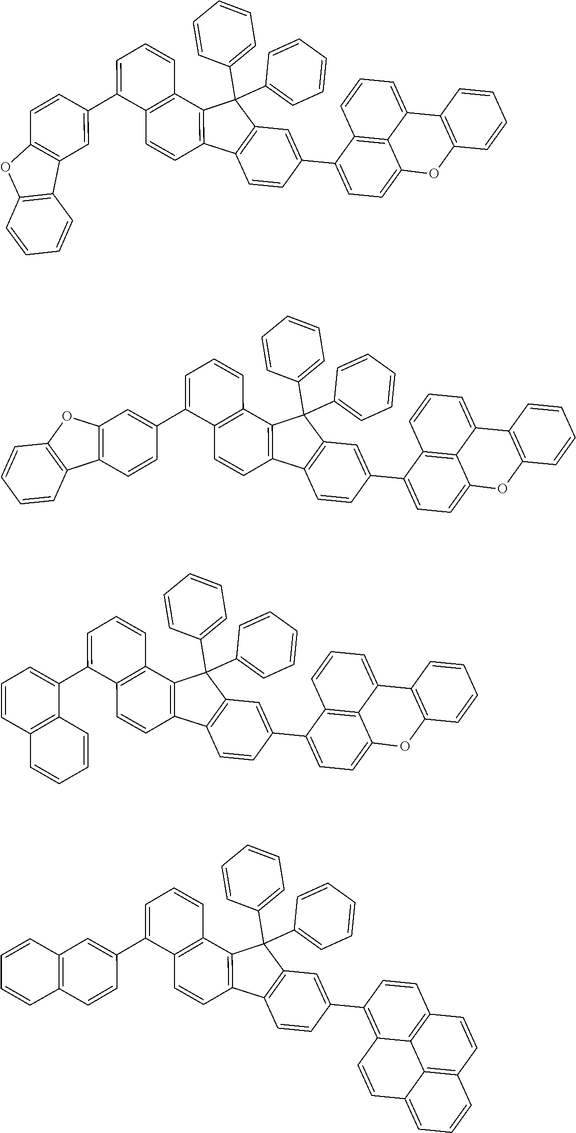

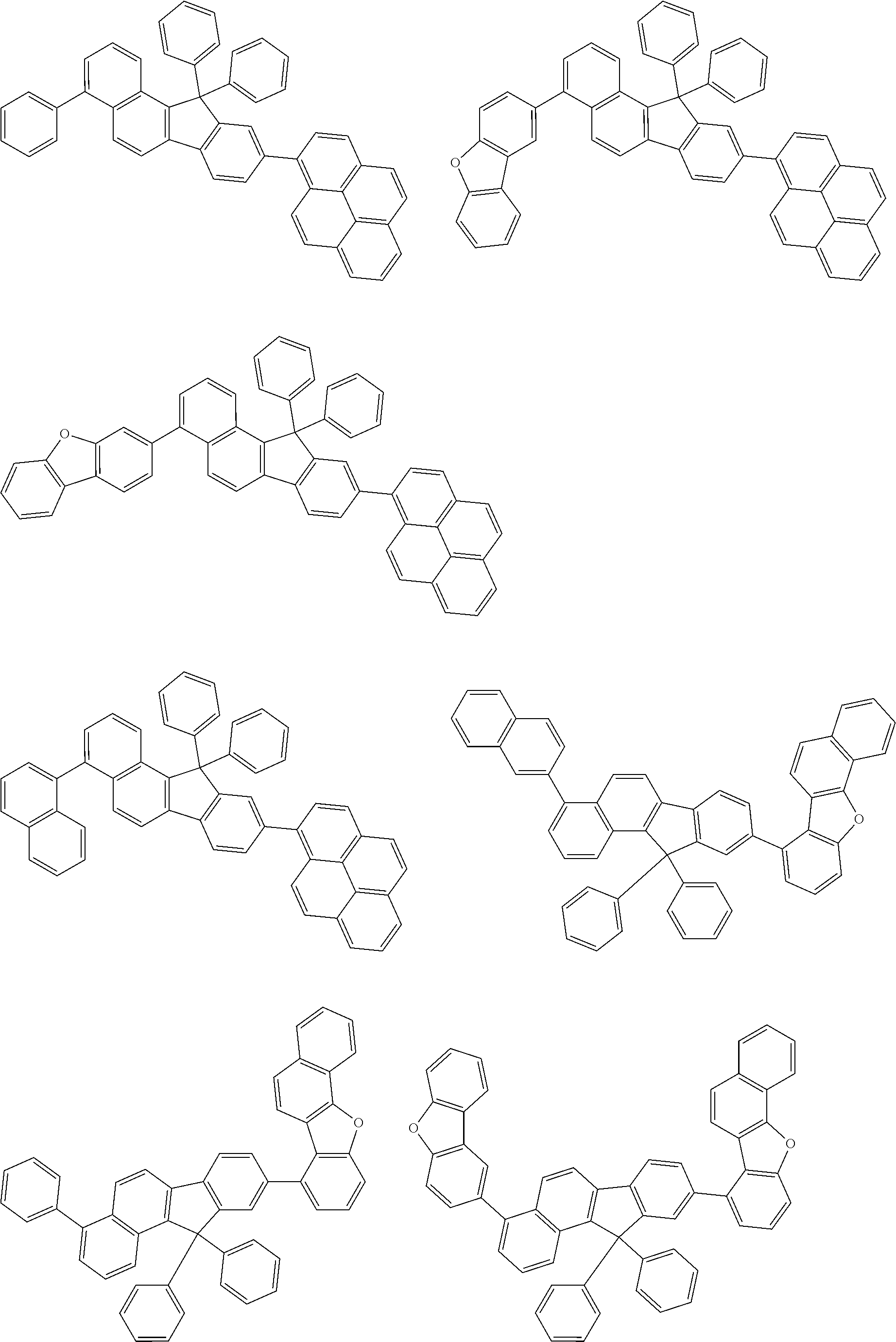

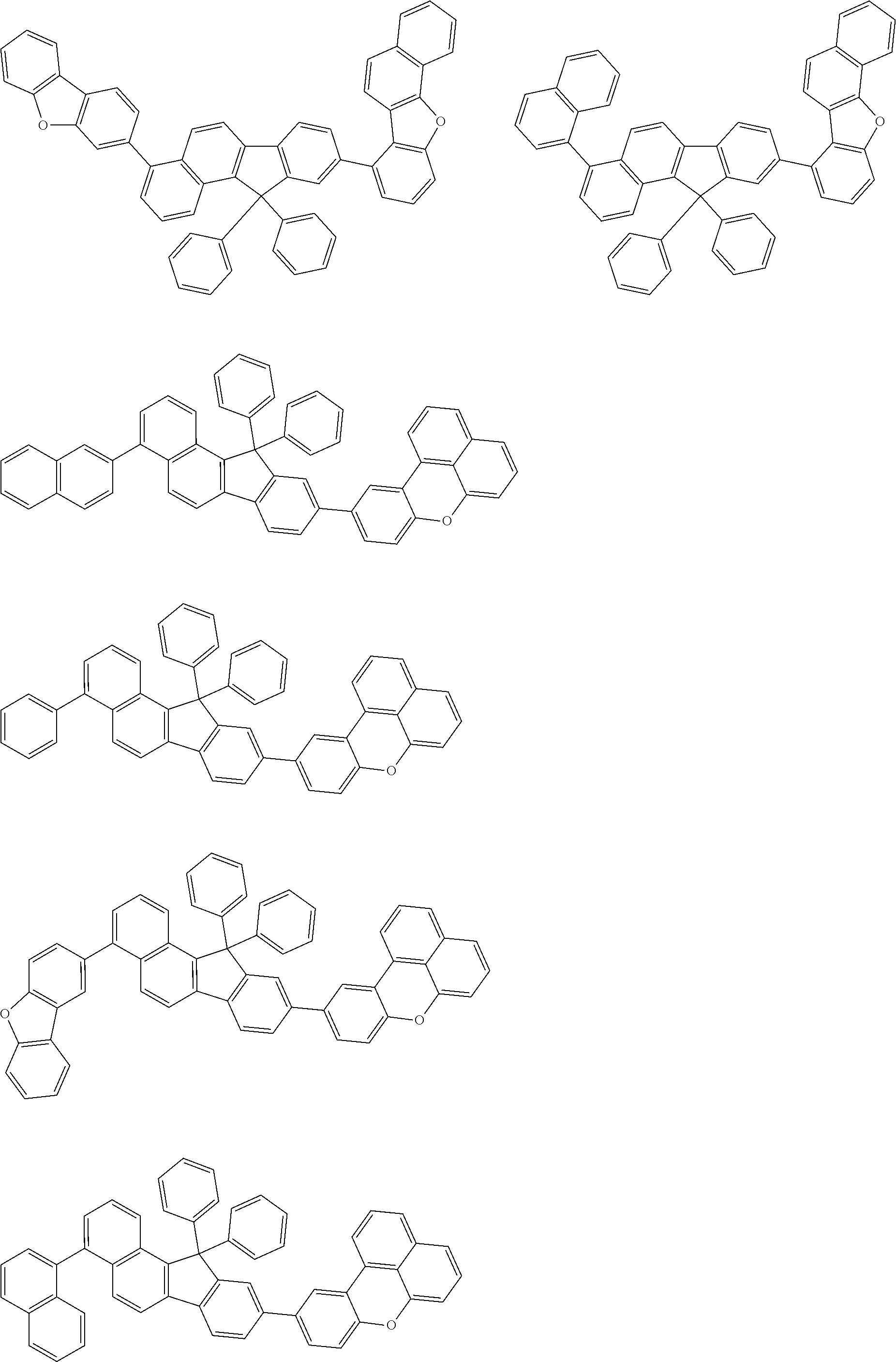

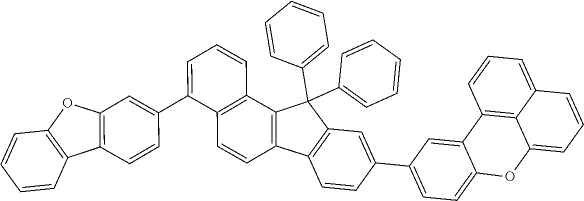 [SMILES: c1ccc(C2(c3ccccc3)c3cc(-c4ccc5c(c4)-c4cccc6cccc(c46)O5)ccc3-c3ccc4c(-c5ccc6c(c5)oc5ccccc56)cccc4c32)cc1]

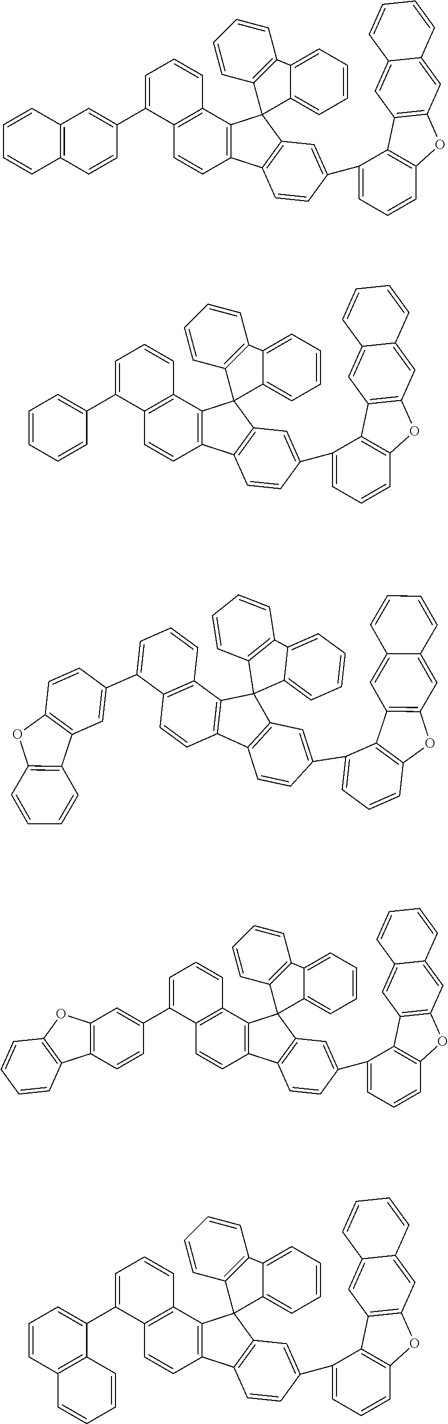 [SMILES: c1ccc2c(c1)-c1ccccc1C21c2cc(-c3cccc4oc5cc6ccccc6cc5c34)ccc2-c2ccc3c(-c4ccc5ccccc5c4)cccc3c21 ; c1ccc(-c2cccc3c4c(ccc23)-c2ccc(-c3cccc5oc6cc7ccccc7cc6c35)cc2C42c3ccccc3-c3ccccc32)cc1 ; c1ccc2c(c1)-c1ccccc1C21c2cc(-c3cccc4oc5cc6ccccc6cc5c34)ccc2-c2ccc3c(-c4ccc5oc6ccccc6c5c4)cccc3c21 ; c1ccc2c(c1)-c1ccccc1C21c2cc(-c3cccc4oc5cc6ccccc6cc5c34)ccc2-c2ccc3c(-c4ccc5c(c4)oc4ccccc45)cccc3c21 ; c1ccc2c(c1)-c1ccccc1C21c2cc(-c3cccc4oc5cc6ccccc6cc5c34)ccc2-c2ccc3c(-c4cccc5ccccc45)cccc3c21]

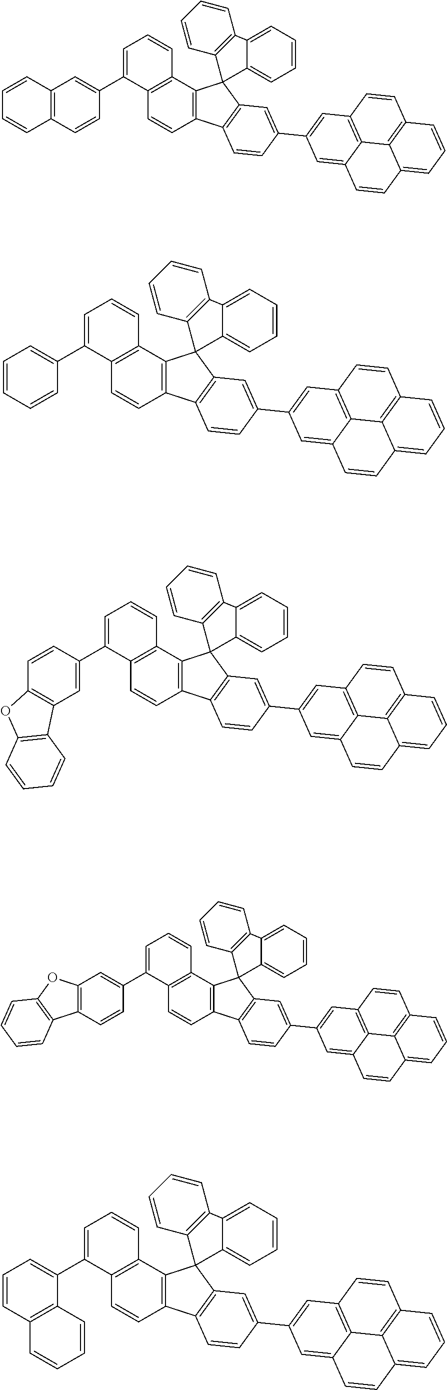

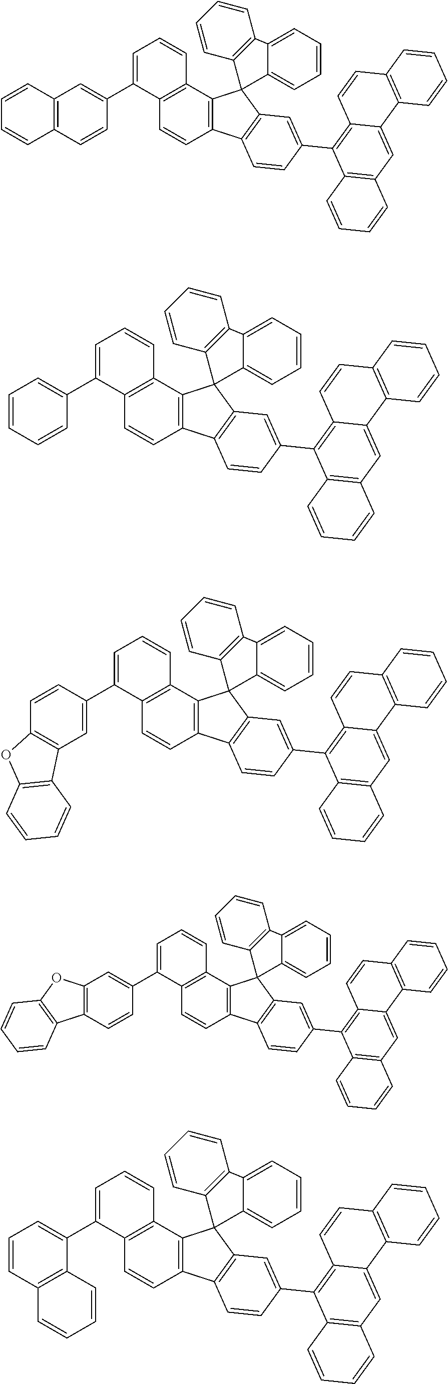

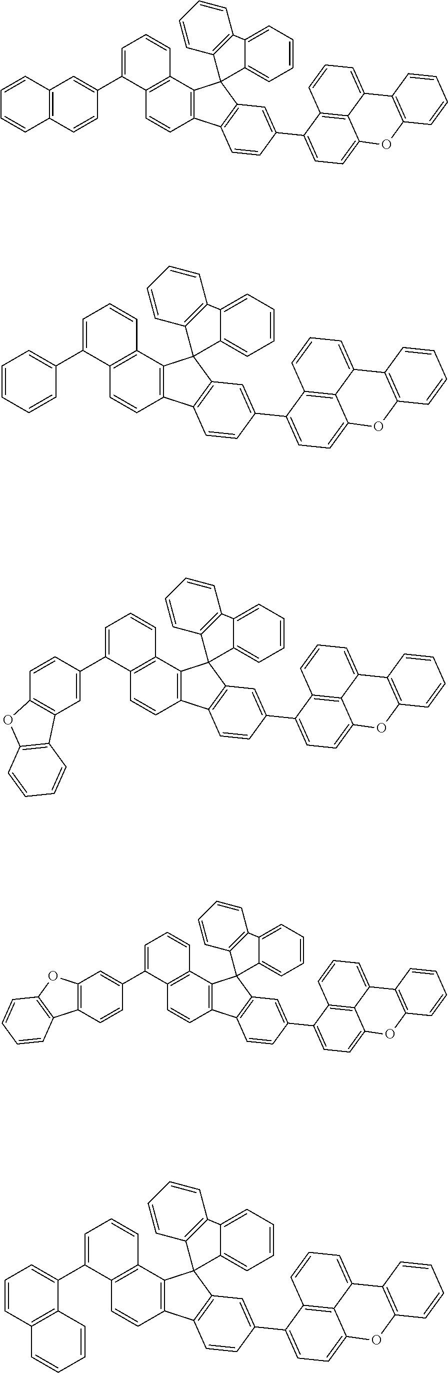

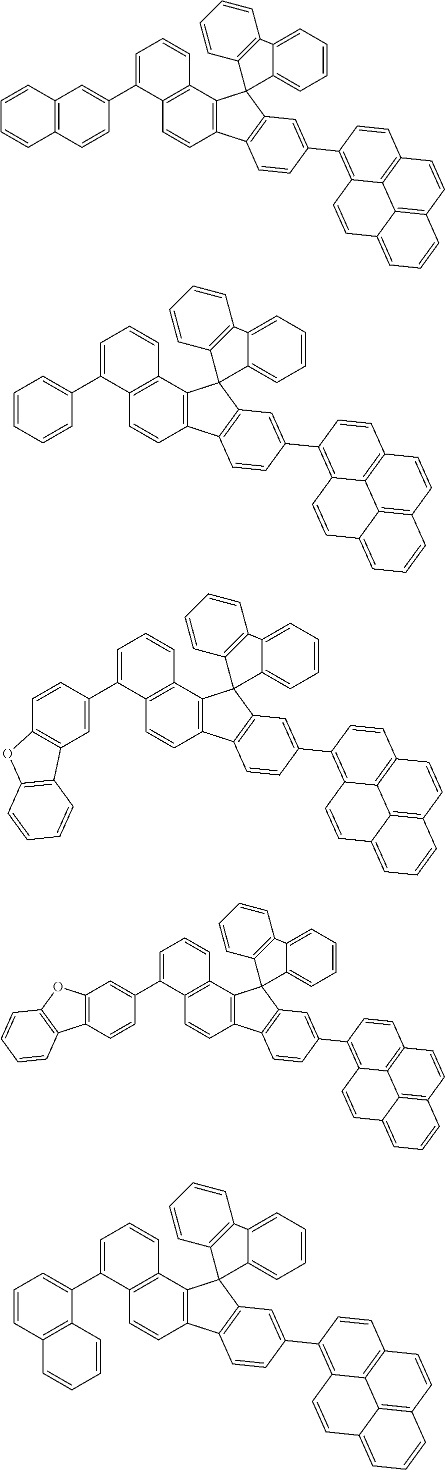

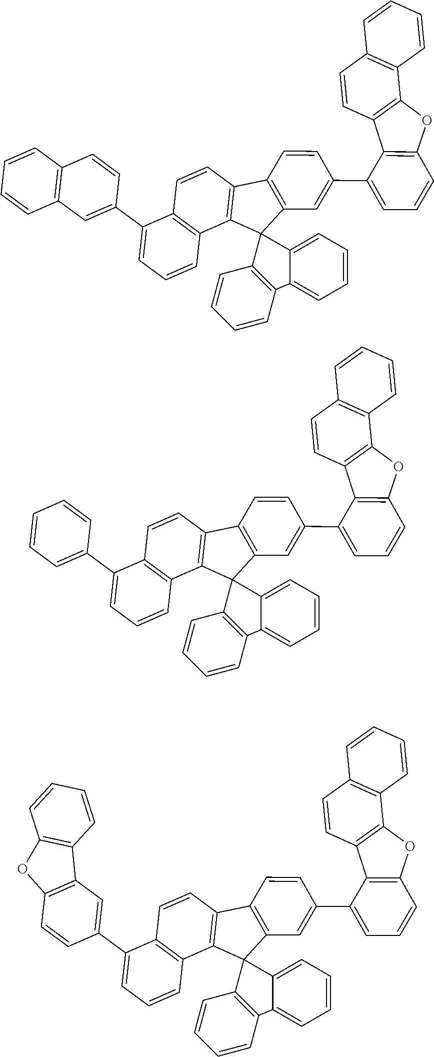 [SMILES: c1ccc2c(c1)-c1ccccc1C21c2cc(-c3cccc4oc5c6ccccc6ccc5c34)ccc2-c2ccc3c(-c4ccc5ccccc5c4)cccc3c21 ; c1ccc(-c2cccc3c4c(ccc23)-c2ccc(-c3cccc5oc6c7ccccc7ccc6c35)cc2C42c3ccccc3-c3ccccc32)cc1 ; c1ccc2c(c1)-c1ccccc1C21c2cc(-c3cccc4oc5c6ccccc6ccc5c34)ccc2-c2ccc3c(-c4ccc5oc6ccccc6c5c4)cccc3c21]

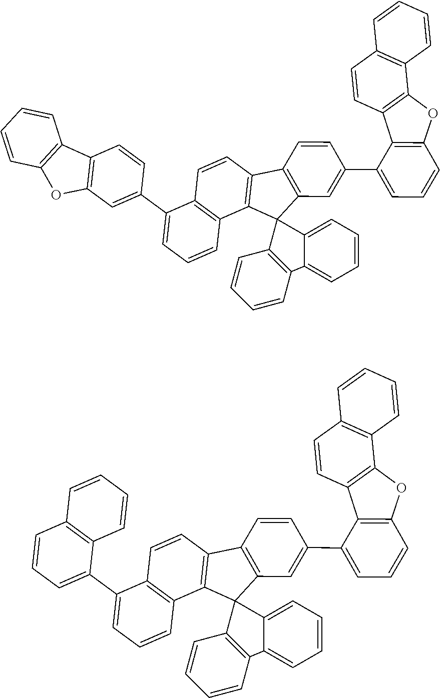 [SMILES: c1ccc2c(c1)-c1ccccc1C21c2cc(-c3cccc4oc5c6ccccc6ccc5c34)ccc2-c2ccc3c(-c4ccc5c(c4)oc4ccccc45)cccc3c21 ; c1ccc2c(c1)-c1ccccc1C21c2cc(-c3cccc4oc5c6ccccc6ccc5c34)ccc2-c2ccc3c(-c4cccc5ccccc45)cccc3c21]

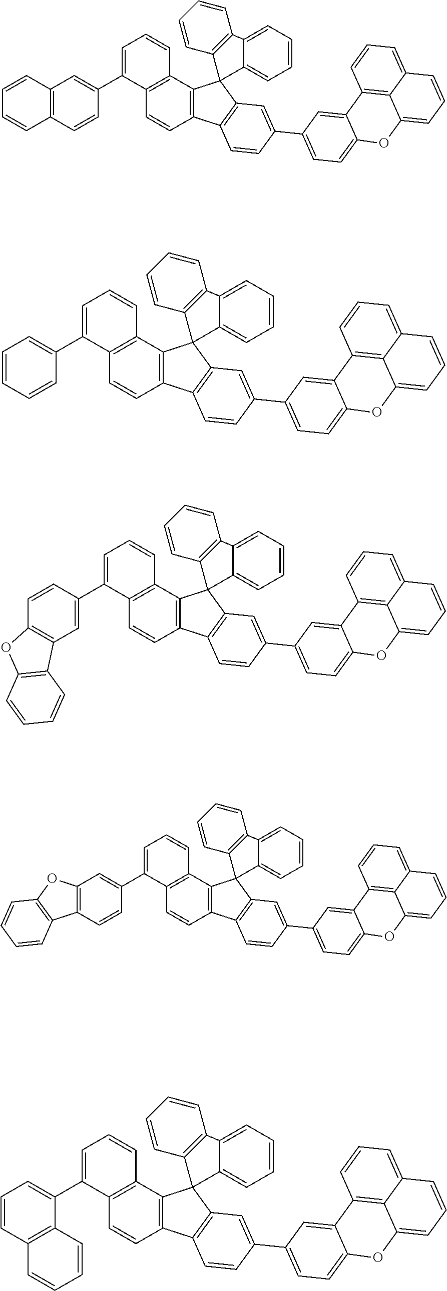

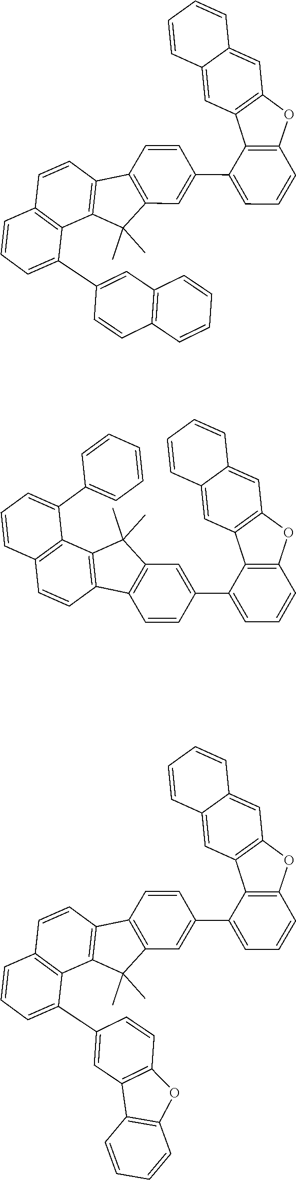 [SMILES: CC1(C)c2cc(-c3cccc4oc5cc6ccccc6cc5c34)ccc2-c2ccc3cccc(-c4ccc5ccccc5c4)c3c21 ; CC1(C)c2cc(-c3cccc4oc5cc6ccccc6cc5c34)ccc2-c2ccc3cccc(-c4ccccc4)c3c21 ; CC1(C)c2cc(-c3cccc4oc5cc6ccccc6cc5c34)ccc2-c2ccc3cccc(-c4ccc5oc6ccccc6c5c4)c3c21]

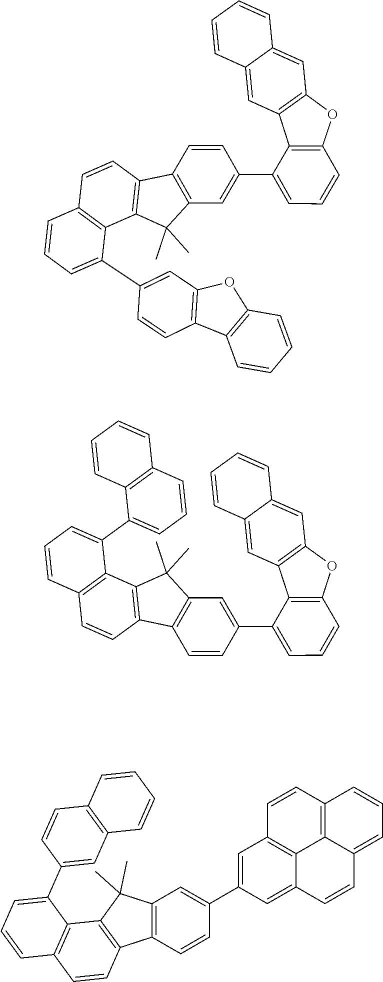

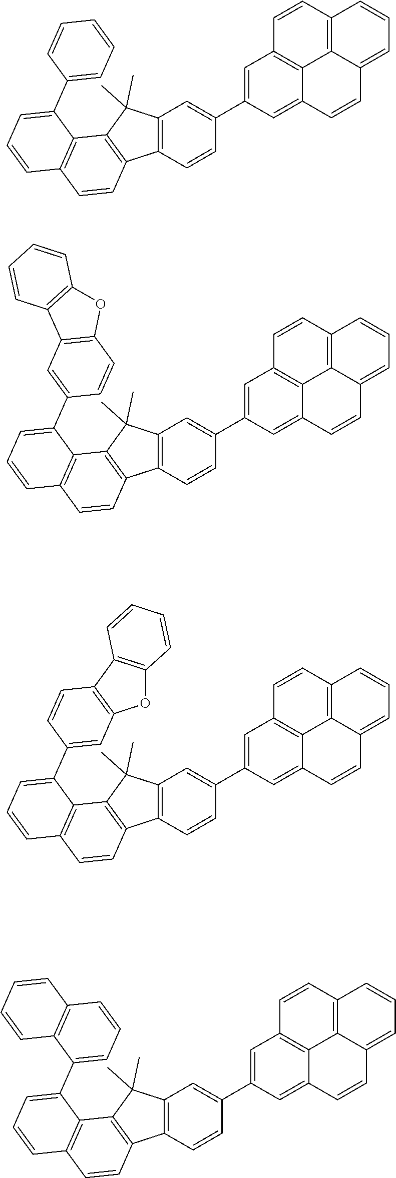

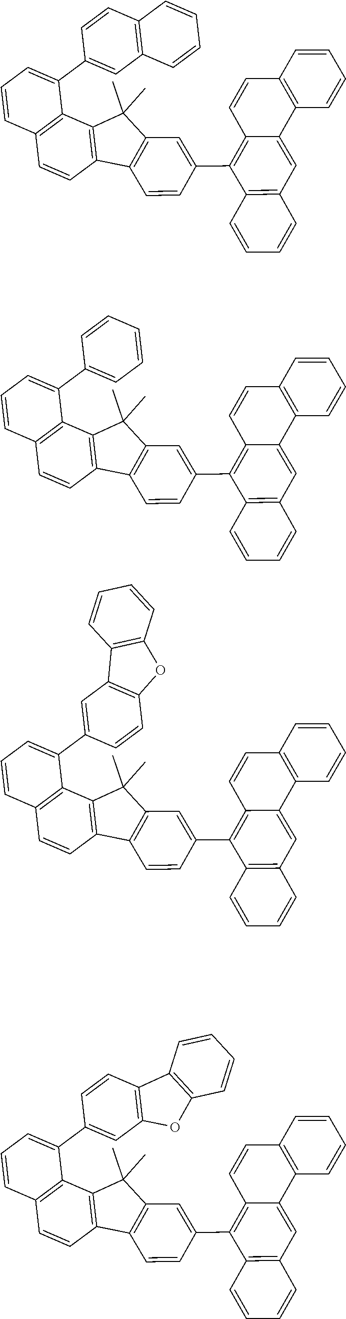

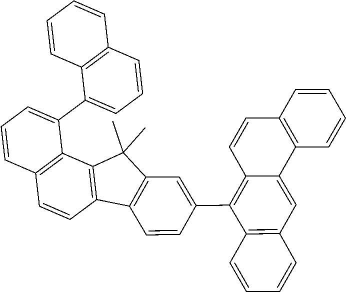 [SMILES: CC1(C)c2cc(-c3c4ccccc4cc4c3ccc3ccccc34)ccc2-c2ccc3cccc(-c4cccc5ccccc45)c3c21]

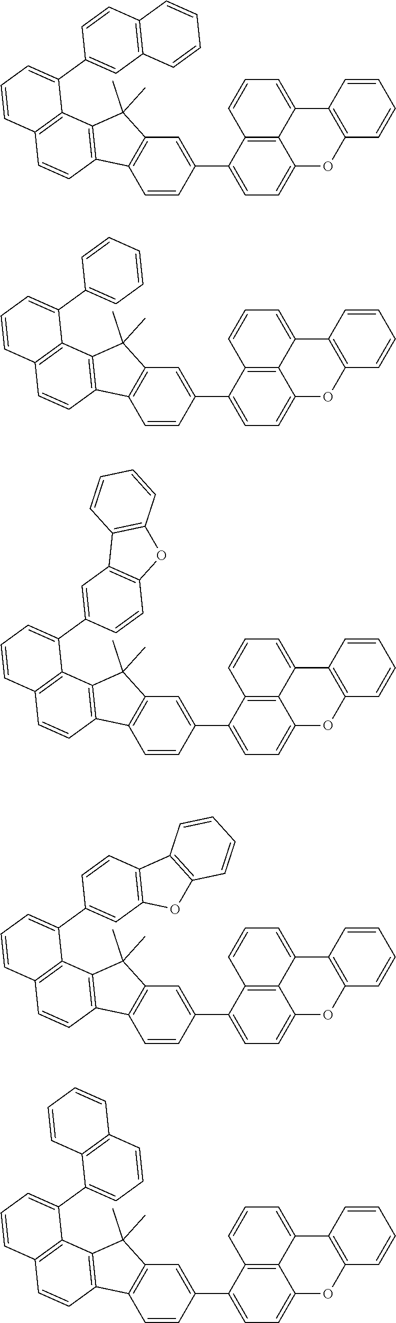

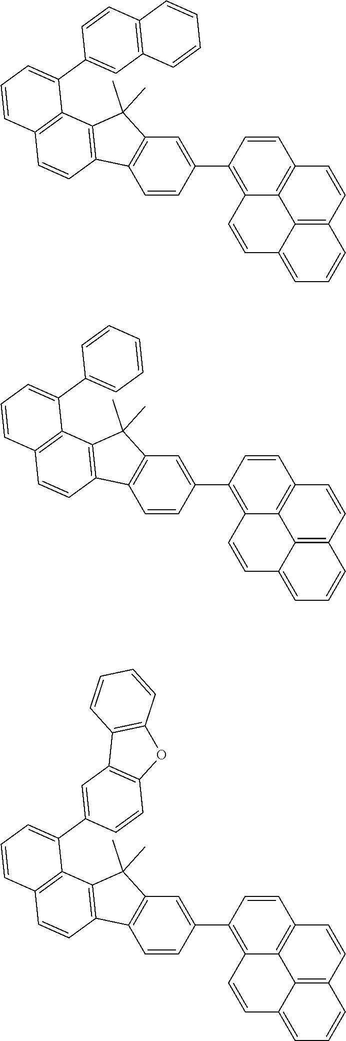

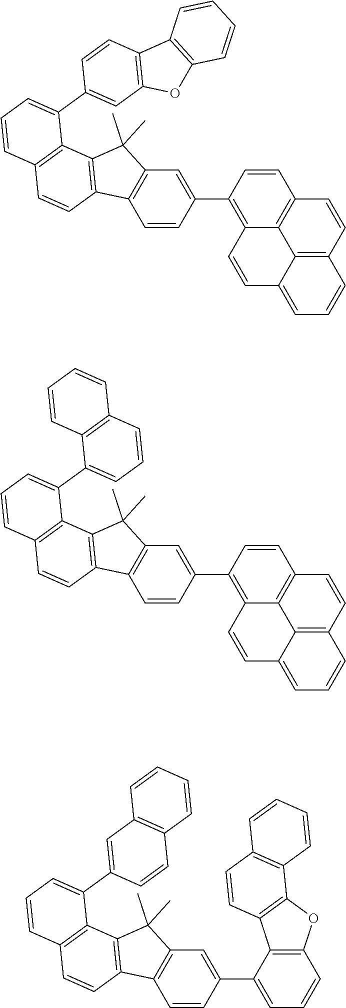

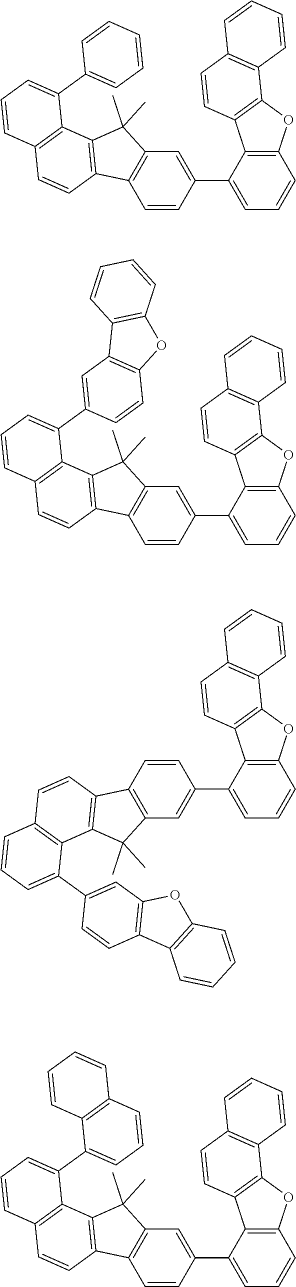 [SMILES: CC1(C)c2cc(-c3cccc4oc5c6ccccc6ccc5c34)ccc2-c2ccc3cccc(-c4ccccc4)c3c21 ; CC1(C)c2cc(-c3cccc4oc5c6ccccc6ccc5c34)ccc2-c2ccc3cccc(-c4ccc5oc6ccccc6c5c4)c3c21 ; CC1(C)c2cc(-c3cccc4oc5c6ccccc6ccc5c34)ccc2-c2ccc3cccc(-c4ccc5c(c4)oc4ccccc45)c3c21 ; CC1(C)c2cc(-c3cccc4oc5c6ccccc6ccc5c34)ccc2-c2ccc3cccc(-c4cccc5ccccc45)c3c21]

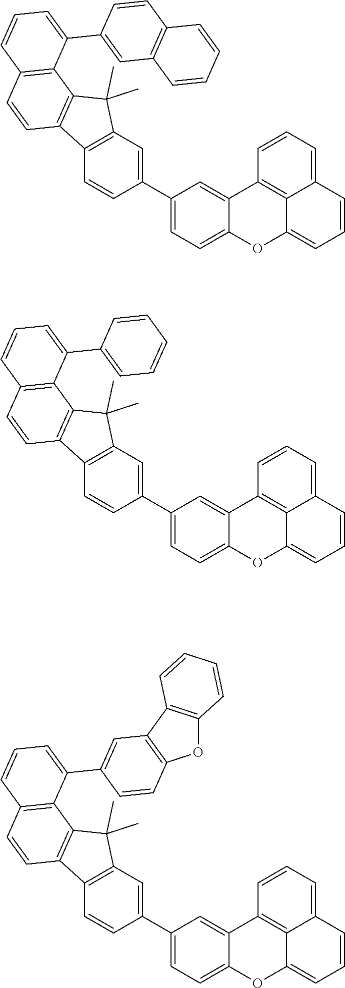 [SMILES: CC1(C)c2cc(-c3ccc4c(c3)-c3cccc5cccc(c35)O4)ccc2-c2ccc3cccc(-c4ccc5ccccc5c4)c3c21 ; CC1(C)c2cc(-c3ccc4c(c3)-c3cccc5cccc(c35)O4)ccc2-c2ccc3cccc(-c4ccccc4)c3c21 ; CC1(C)c2cc(-c3ccc4c(c3)-c3cccc5cccc(c35)O4)ccc2-c2ccc3cccc(-c4ccc5oc6ccccc6c5c4)c3c21]

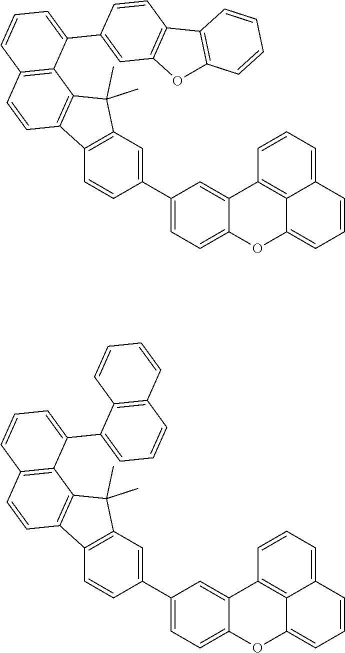 [SMILES: CC1(C)c2cc(-c3ccc4c(c3)-c3cccc5cccc(c35)O4)ccc2-c2ccc3cccc(-c4ccc5c(c4)oc4ccccc45)c3c21 ; CC1(C)c2cc(-c3ccc4c(c3)-c3cccc5cccc(c35)O4)ccc2-c2ccc3cccc(-c4cccc5ccccc45)c3c21]

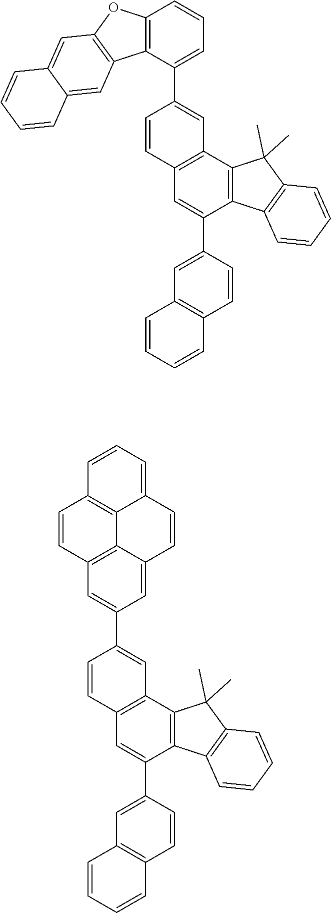

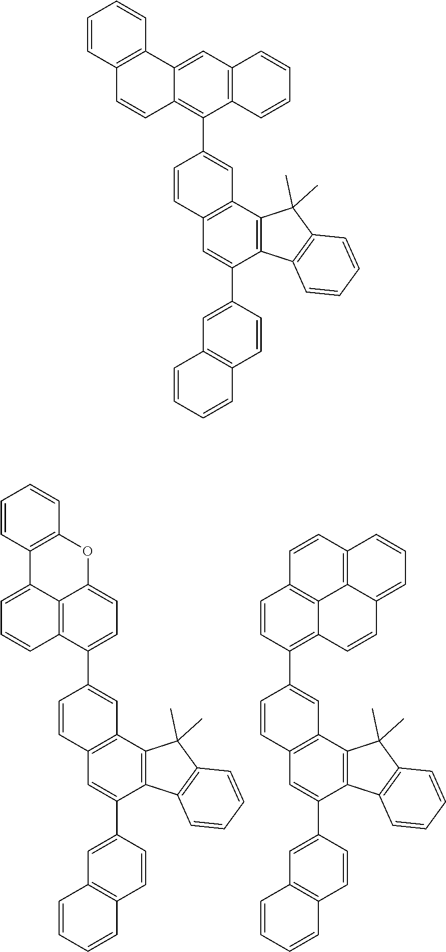

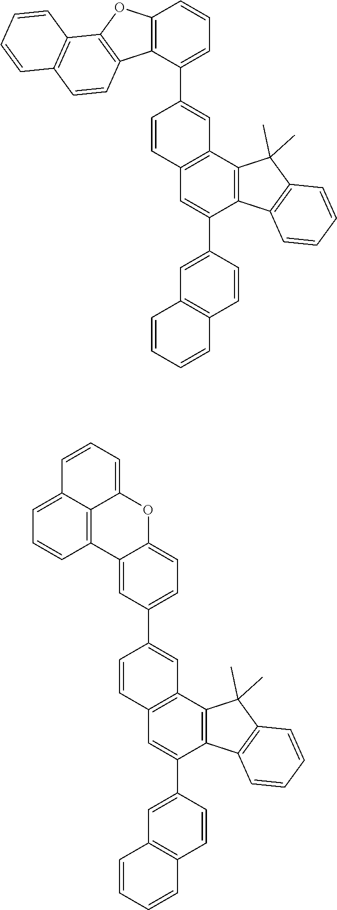 [SMILES: CC1(C)c2ccccc2-c2c(-c3ccc4ccccc4c3)cc3ccc(-c4cccc5oc6c7ccccc7ccc6c45)cc3c21 ; CC1(C)c2ccccc2-c2c(-c3ccc4ccccc4c3)cc3ccc(-c4ccc5c(c4)-c4cccc6cccc(c46)O5)cc3c21]

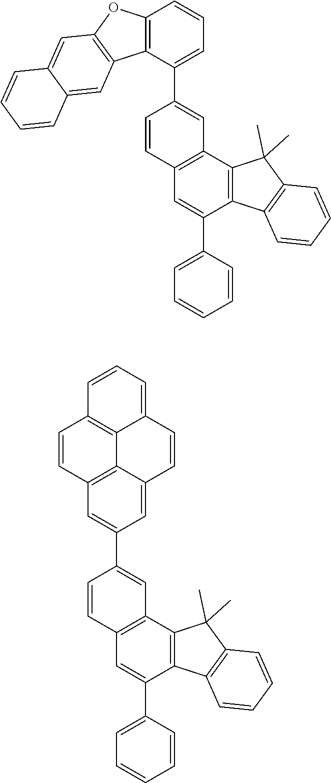

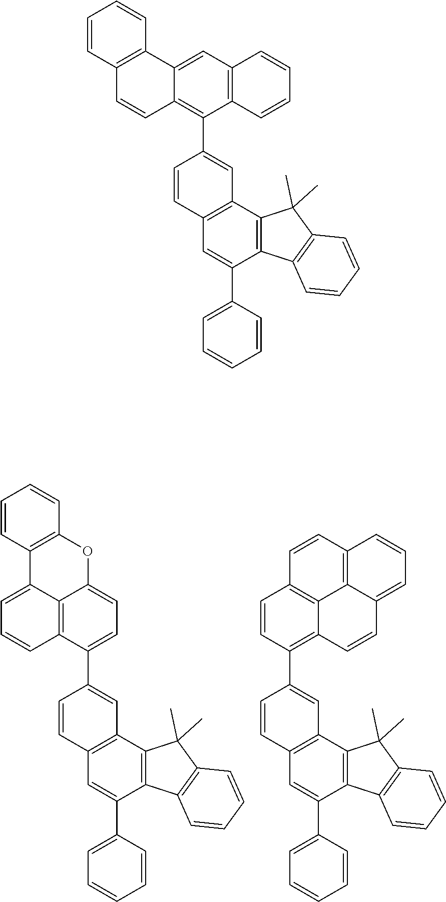

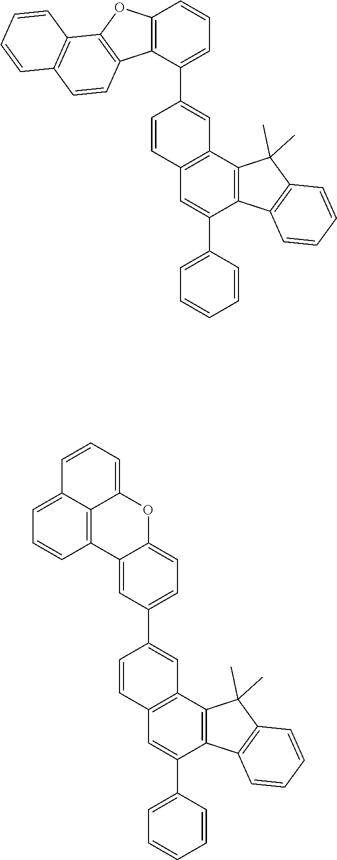 [SMILES: CC1(C)c2ccccc2-c2c(-c3ccccc3)cc3ccc(-c4cccc5oc6c7ccccc7ccc6c45)cc3c21 ; CC1(C)c2ccccc2-c2c(-c3ccccc3)cc3ccc(-c4ccc5c(c4)-c4cccc6cccc(c46)O5)cc3c21]

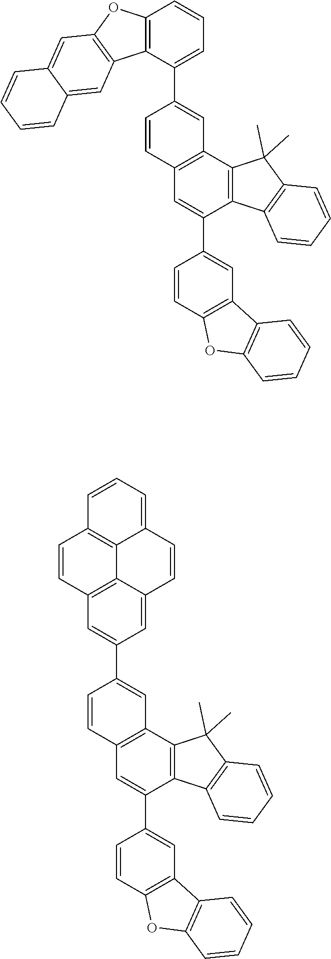 [SMILES: CC1(C)c2ccccc2-c2c(-c3ccc4oc5ccccc5c4c3)cc3ccc(-c4cccc5oc6cc7ccccc7cc6c45)cc3c21 ; CC1(C)c2ccccc2-c2c(-c3ccc4oc5ccccc5c4c3)cc3ccc(-c4cc5ccc6cccc7ccc(c4)c5c67)cc3c21]

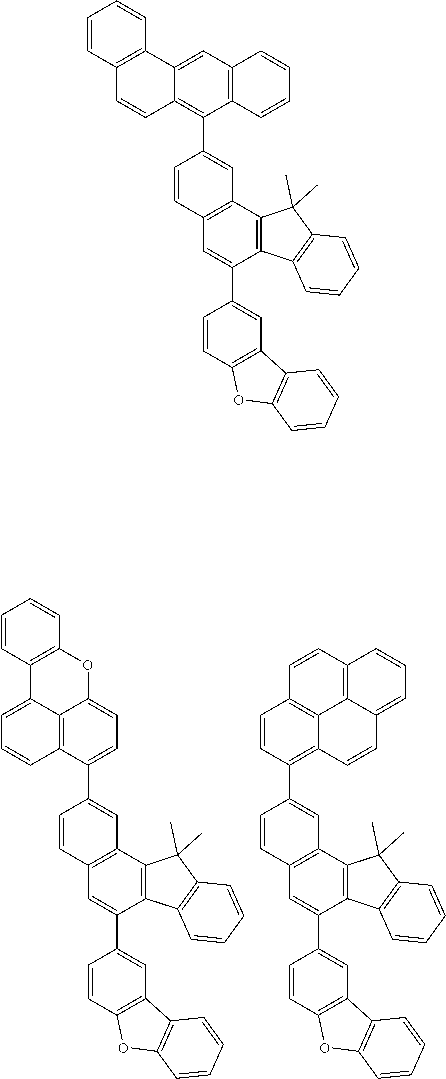 [SMILES: CC1(C)c2ccccc2-c2c(-c3ccc4oc5ccccc5c4c3)cc3ccc(-c4c5ccccc5cc5c4ccc4ccccc45)cc3c21 ; CC1(C)c2ccccc2-c2c(-c3ccc4oc5ccccc5c4c3)cc3ccc(-c4ccc5c6c(cccc46)-c4ccccc4O5)cc3c21 ; CC1(C)c2ccccc2-c2c(-c3ccc4oc5ccccc5c4c3)cc3ccc(-c4ccc5ccc6cccc7ccc4c5c67)cc3c21]

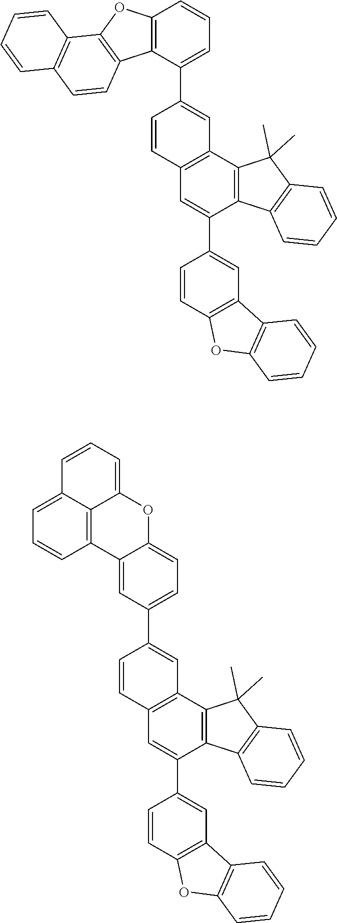 [SMILES: CC1(C)c2ccccc2-c2c(-c3ccc4oc5ccccc5c4c3)cc3ccc(-c4cccc5oc6c7ccccc7ccc6c45)cc3c21 ; CC1(C)c2ccccc2-c2c(-c3ccc4oc5ccccc5c4c3)cc3ccc(-c4ccc5c(c4)-c4cccc6cccc(c46)O5)cc3c21]

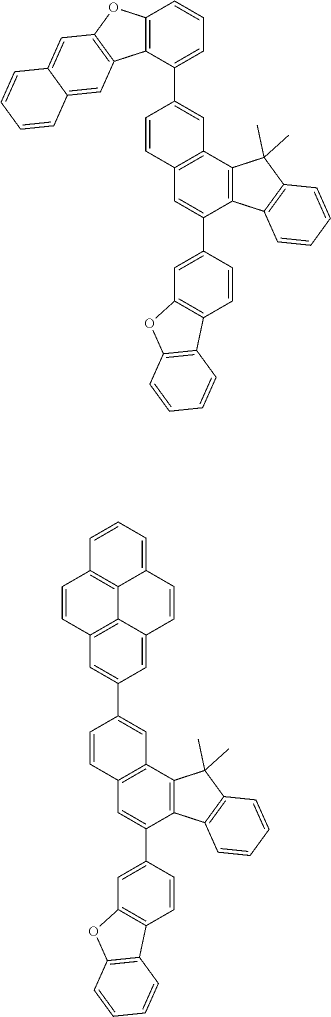 [SMILES: CC1(C)c2ccccc2-c2c(-c3ccc4c(c3)oc3ccccc34)cc3ccc(-c4cccc5oc6cc7ccccc7cc6c45)cc3c21 ; CC1(C)c2ccccc2-c2c(-c3ccc4c(c3)oc3ccccc34)cc3ccc(-c4cc5ccc6cccc7ccc(c4)c5c67)cc3c21]

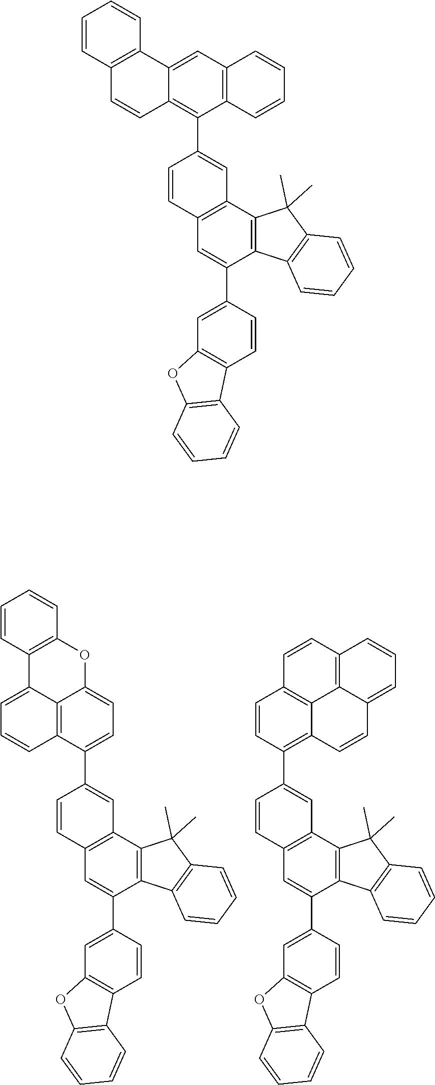 [SMILES: CC1(C)c2ccccc2-c2c(-c3ccc4c(c3)oc3ccccc34)cc3ccc(-c4c5ccccc5cc5c4ccc4ccccc45)cc3c21 ; CC1(C)c2ccccc2-c2c(-c3ccc4c(c3)oc3ccccc34)cc3ccc(-c4ccc5c6c(cccc46)-c4ccccc4O5)cc3c21 ; CC1(C)c2ccccc2-c2c(-c3ccc4c(c3)oc3ccccc34)cc3ccc(-c4ccc5ccc6cccc7ccc4c5c67)cc3c21]

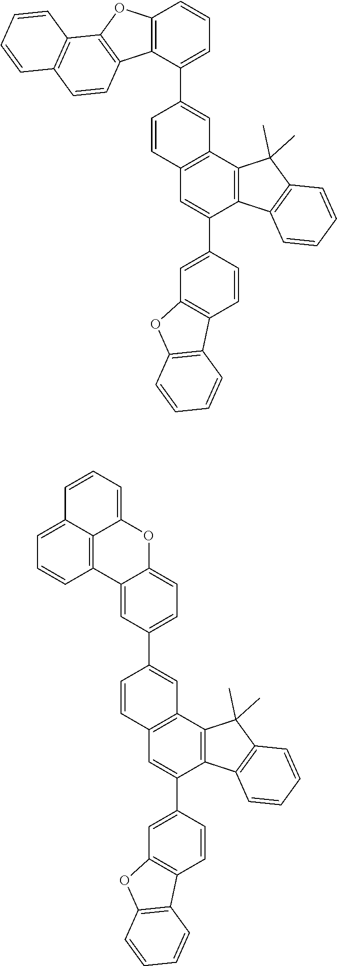 [SMILES: CC1(C)c2ccccc2-c2c(-c3ccc4c(c3)oc3ccccc34)cc3ccc(-c4cccc5oc6c7ccccc7ccc6c45)cc3c21 ; CC1(C)c2ccccc2-c2c(-c3ccc4c(c3)oc3ccccc34)cc3ccc(-c4ccc5c(c4)-c4cccc6cccc(c46)O5)cc3c21]

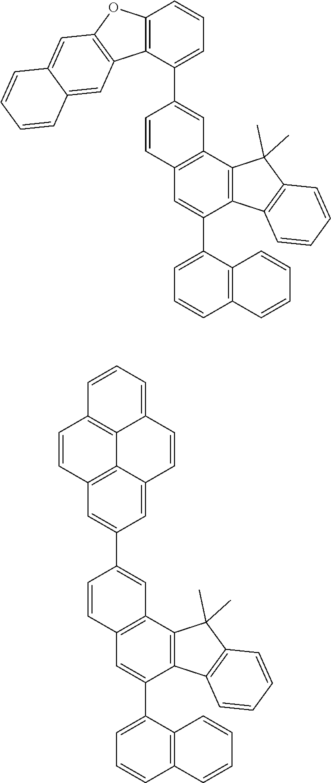

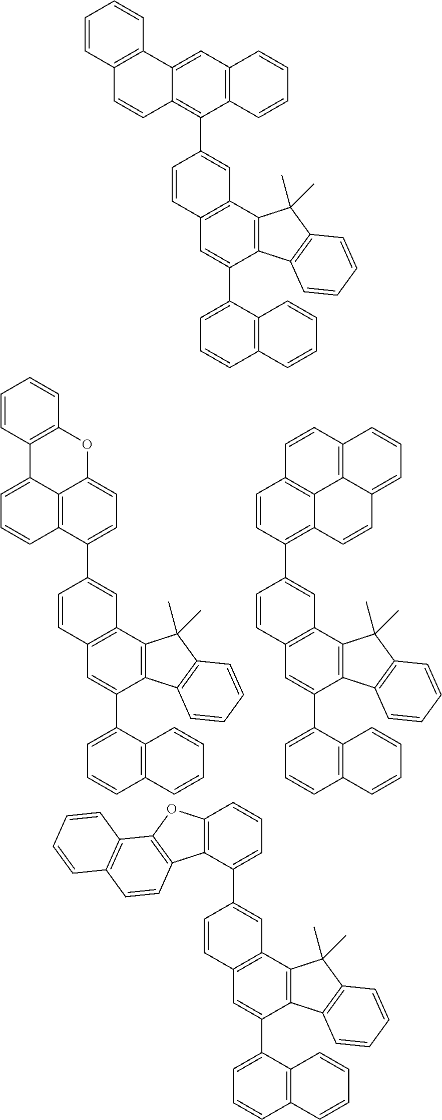

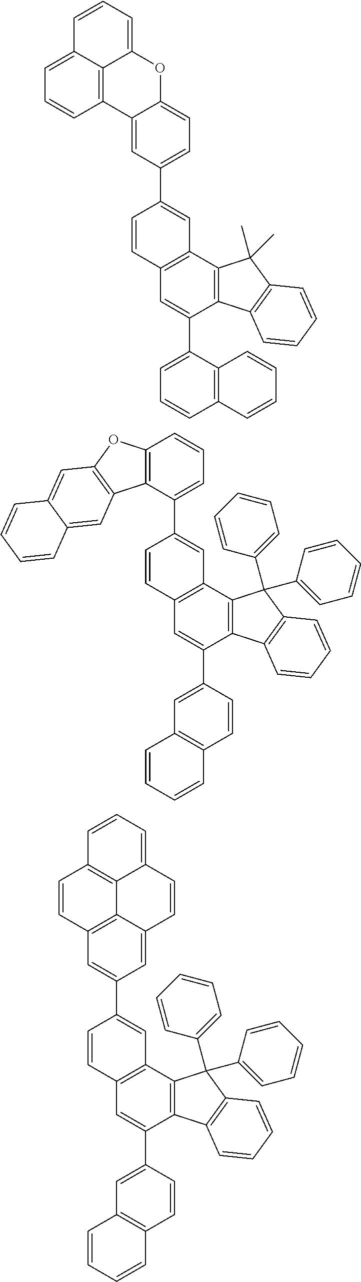

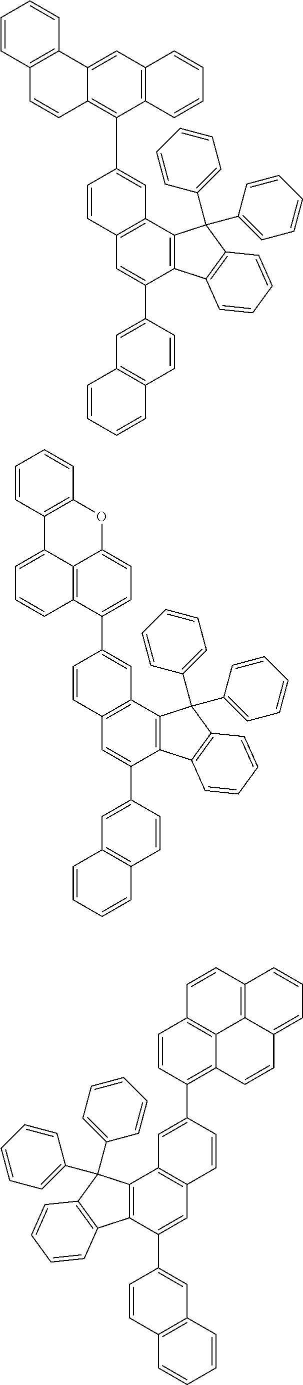

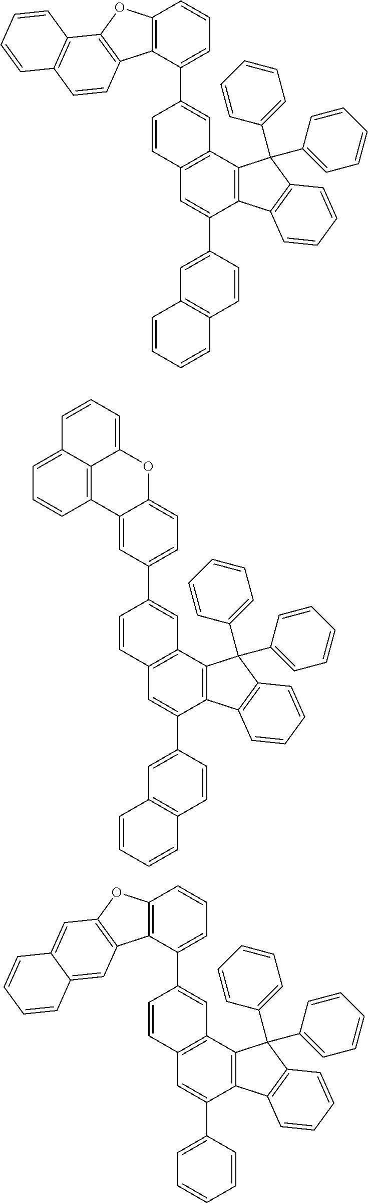

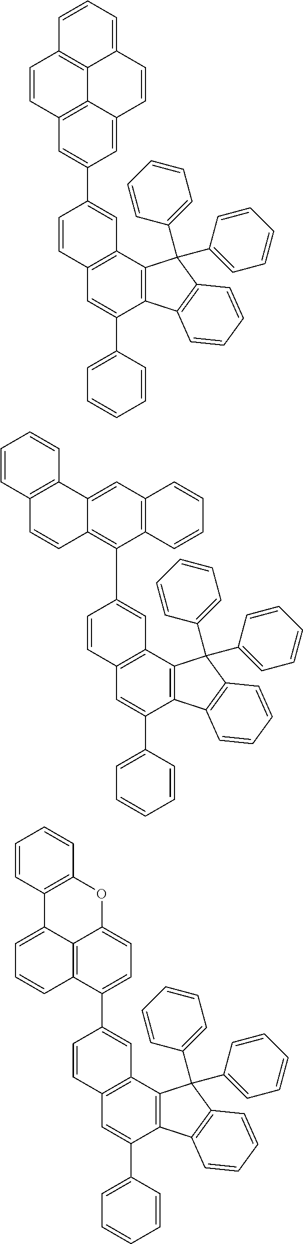

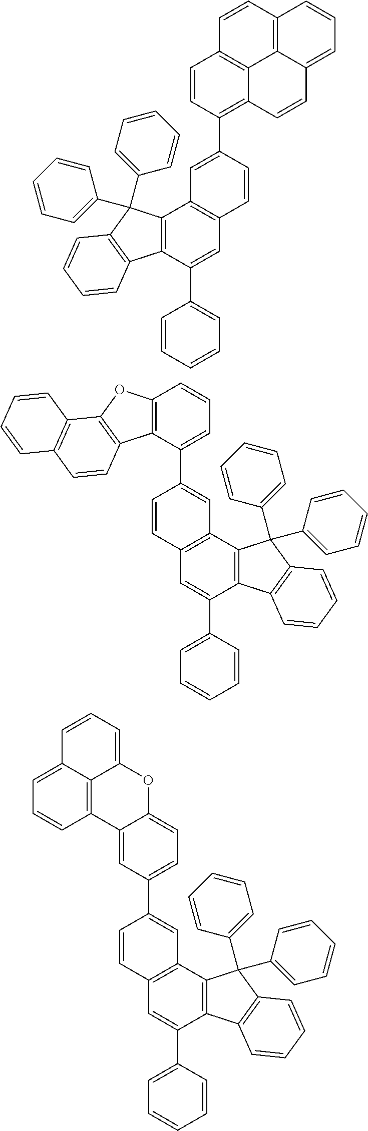

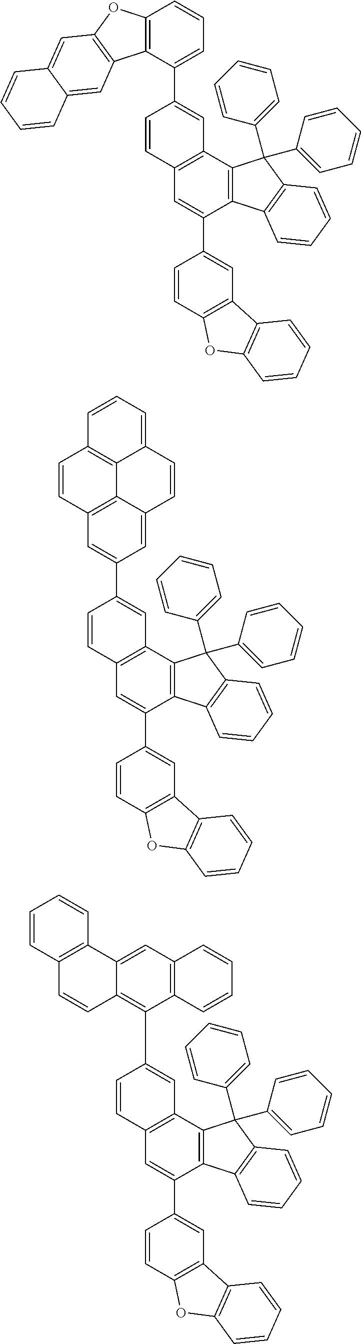 [SMILES: c1ccc(C2(c3ccccc3)c3ccccc3-c3c(-c4ccc5oc6ccccc6c5c4)cc4ccc(-c5cccc6oc7cc8ccccc8cc7c56)cc4c32)cc1 ; c1ccc(C2(c3ccccc3)c3ccccc3-c3c(-c4ccc5oc6ccccc6c5c4)cc4ccc(-c5cc6ccc7cccc8ccc(c5)c6c78)cc4c32)cc1 ; c1ccc(C2(c3ccccc3)c3ccccc3-c3c(-c4ccc5oc6ccccc6c5c4)cc4ccc(-c5c6ccccc6cc6c5ccc5ccccc56)cc4c32)cc1]

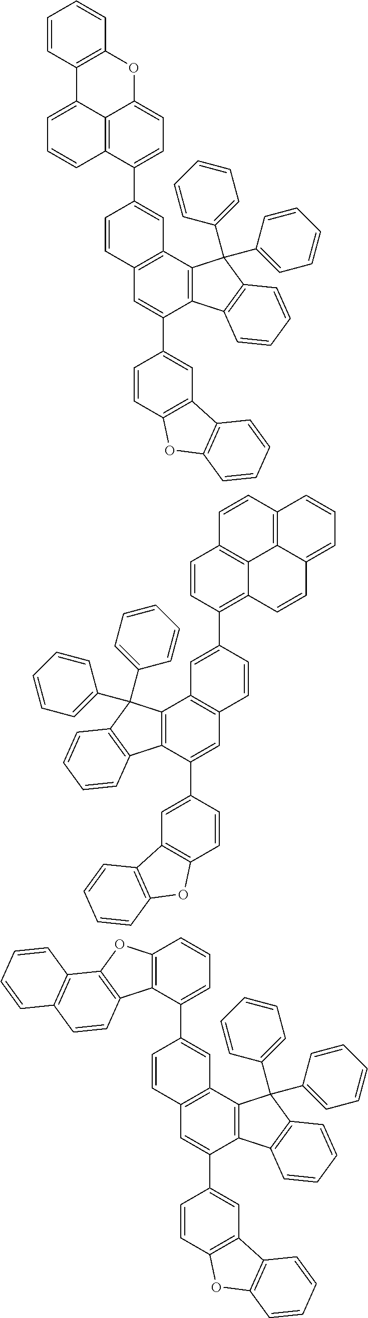

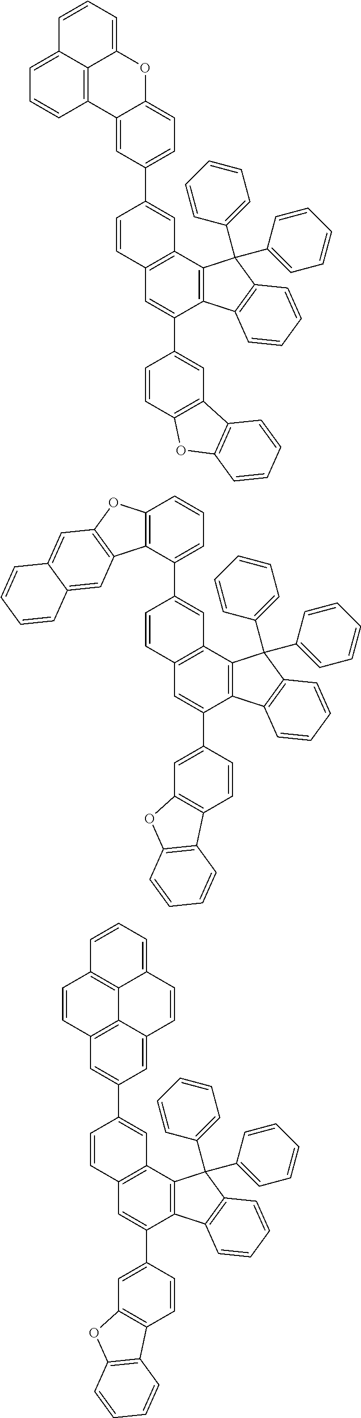

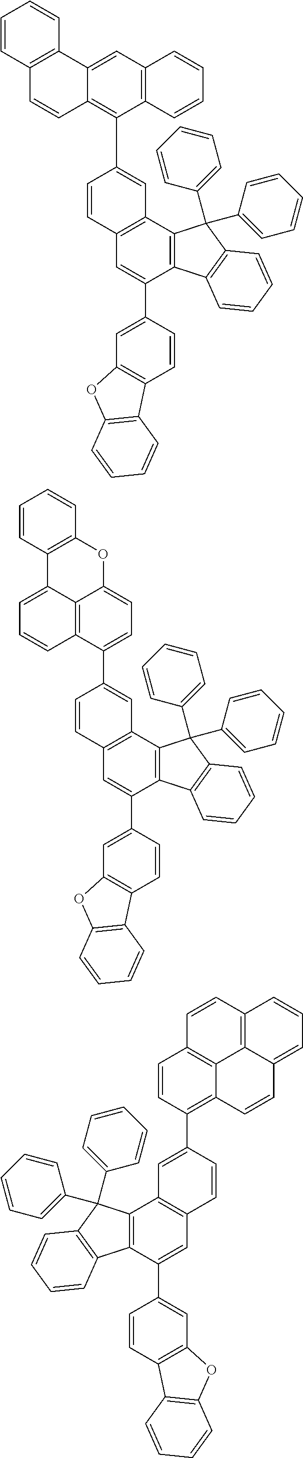

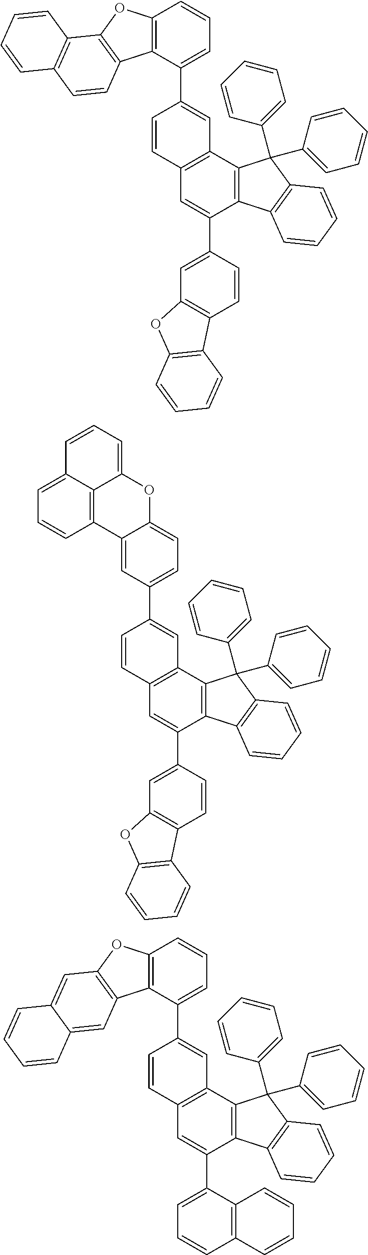

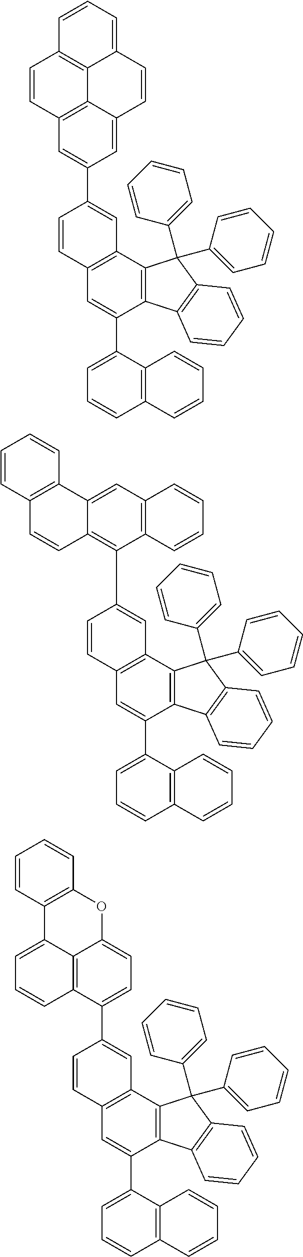

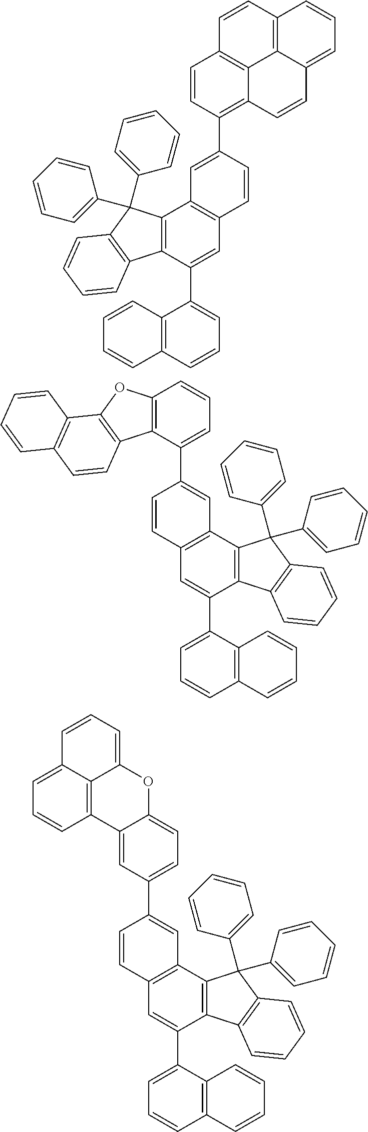

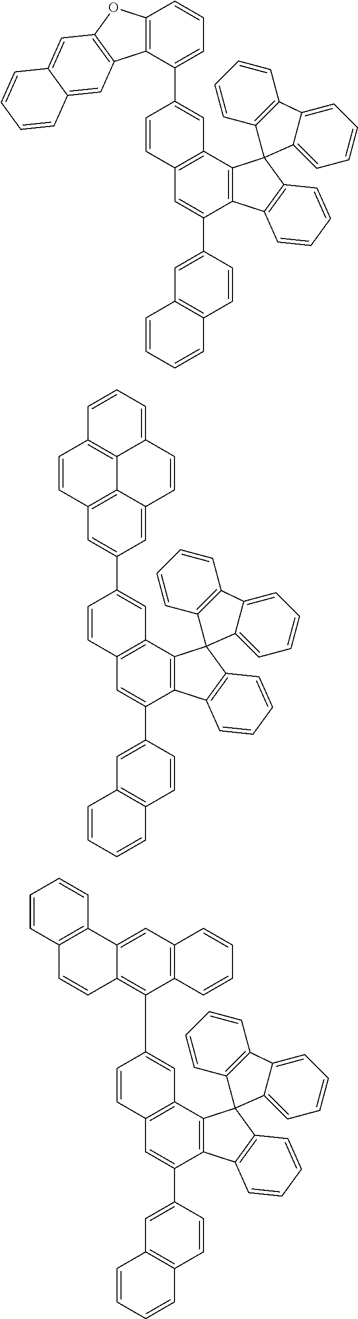

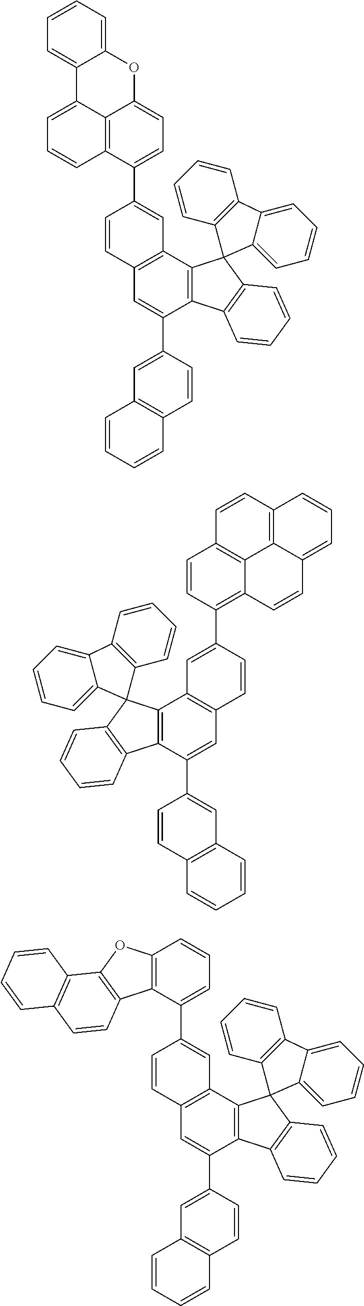

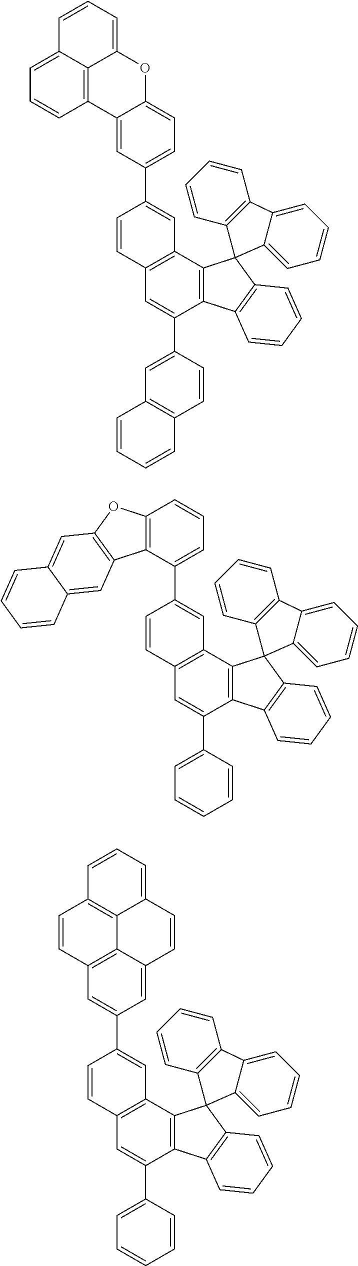

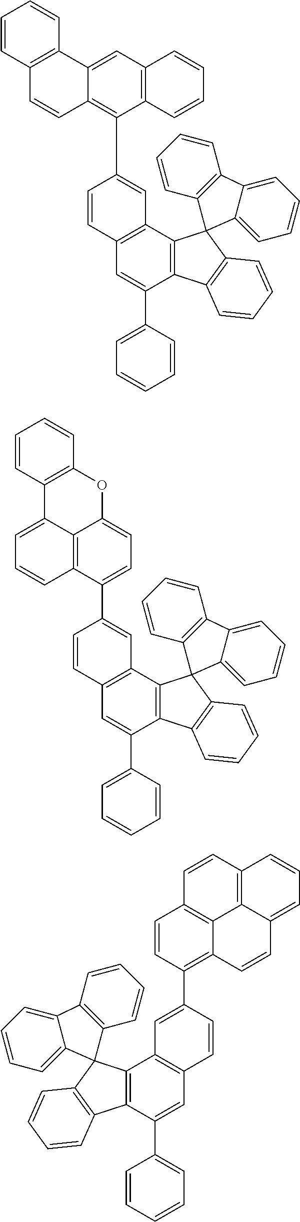

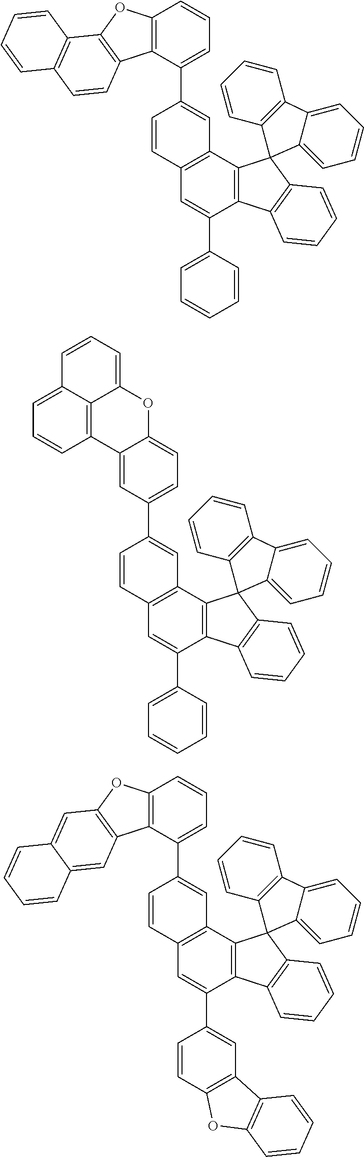

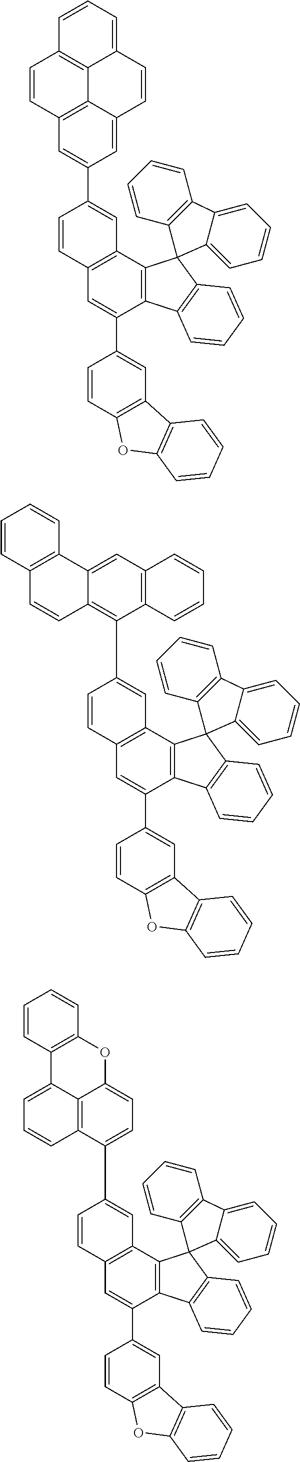 [SMILES: c1ccc2c(c1)-c1ccccc1C21c2ccccc2-c2c(-c3ccc4oc5ccccc5c4c3)cc3ccc(-c4cc5ccc6cccc7ccc(c4)c5c67)cc3c21 ; c1ccc2c(c1)-c1ccccc1C21c2ccccc2-c2c(-c3ccc4oc5ccccc5c4c3)cc3ccc(-c4c5ccccc5cc5c4ccc4ccccc45)cc3c21 ; c1ccc2c(c1)Oc1ccc(-c3ccc4cc(-c5ccc6oc7ccccc7c6c5)c5c(c4c3)C3(c4ccccc4-c4ccccc43)c3ccccc3-5)c3cccc-2c13]

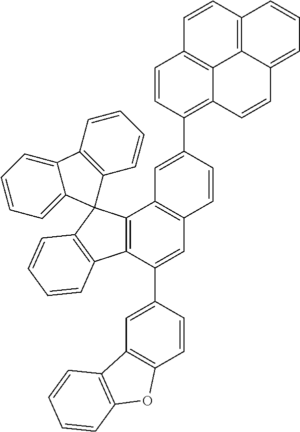 [SMILES: c1ccc2c(c1)-c1ccccc1C21c2ccccc2-c2c(-c3ccc4oc5ccccc5c4c3)cc3ccc(-c4ccc5ccc6cccc7ccc4c5c67)cc3c21]

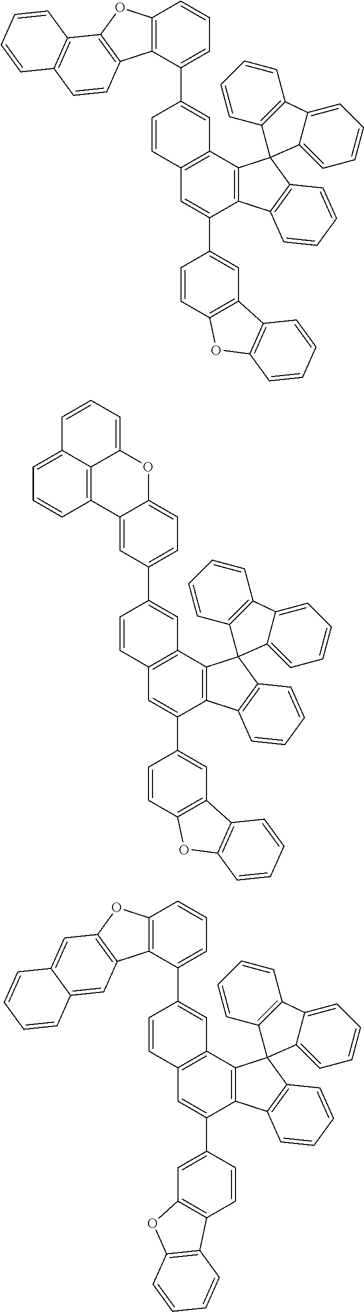 [SMILES: c1ccc2c(c1)-c1ccccc1C21c2ccccc2-c2c(-c3ccc4oc5ccccc5c4c3)cc3ccc(-c4cccc5oc6c7ccccc7ccc6c45)cc3c21 ; c1ccc2c(c1)-c1ccccc1C21c2ccccc2-c2c(-c3ccc4oc5ccccc5c4c3)cc3ccc(-c4ccc5c(c4)-c4cccc6cccc(c46)O5)cc3c21 ; c1ccc2c(c1)-c1ccccc1C21c2ccccc2-c2c(-c3ccc4c(c3)oc3ccccc34)cc3ccc(-c4cccc5oc6cc7ccccc7cc6c45)cc3c21]

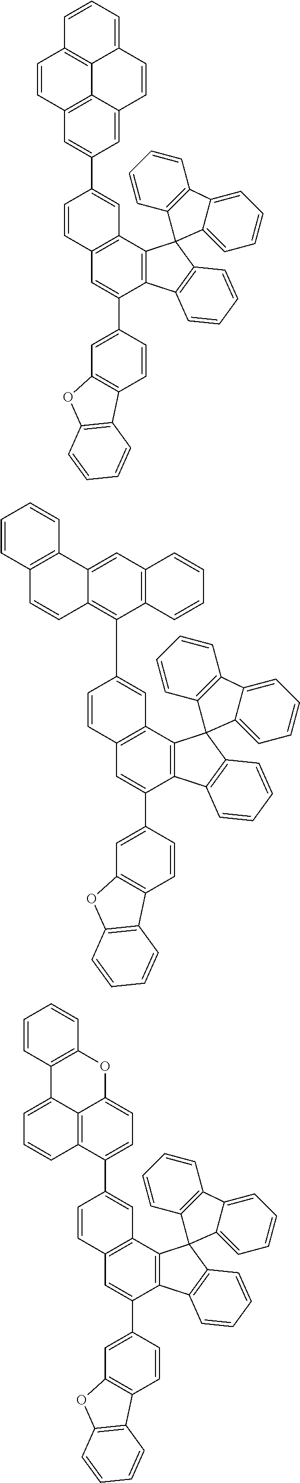 [SMILES: c1ccc2c(c1)-c1ccccc1C21c2ccccc2-c2c(-c3ccc4c(c3)oc3ccccc34)cc3ccc(-c4cc5ccc6cccc7ccc(c4)c5c67)cc3c21 ; c1ccc2c(c1)-c1ccccc1C21c2ccccc2-c2c(-c3ccc4c(c3)oc3ccccc34)cc3ccc(-c4c5ccccc5cc5c4ccc4ccccc45)cc3c21 ; c1ccc2c(c1)Oc1ccc(-c3ccc4cc(-c5ccc6c(c5)oc5ccccc56)c5c(c4c3)C3(c4ccccc4-c4ccccc43)c3ccccc3-5)c3cccc-2c13]

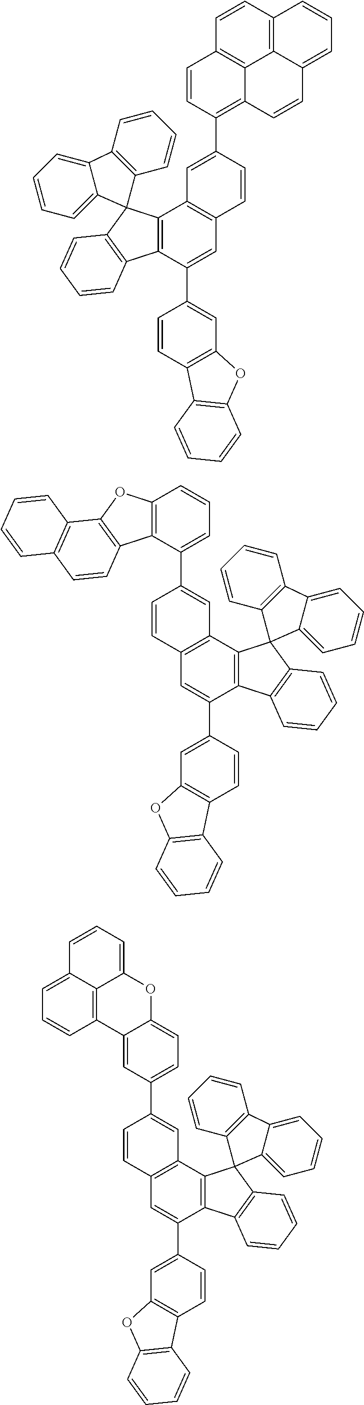

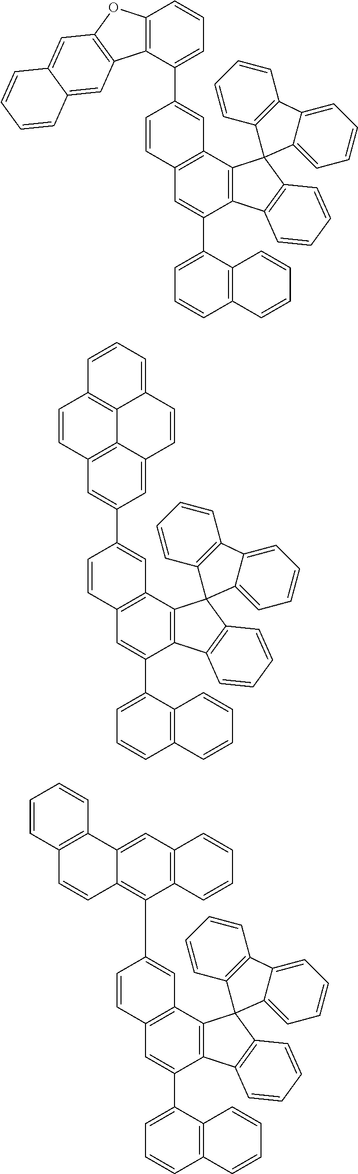

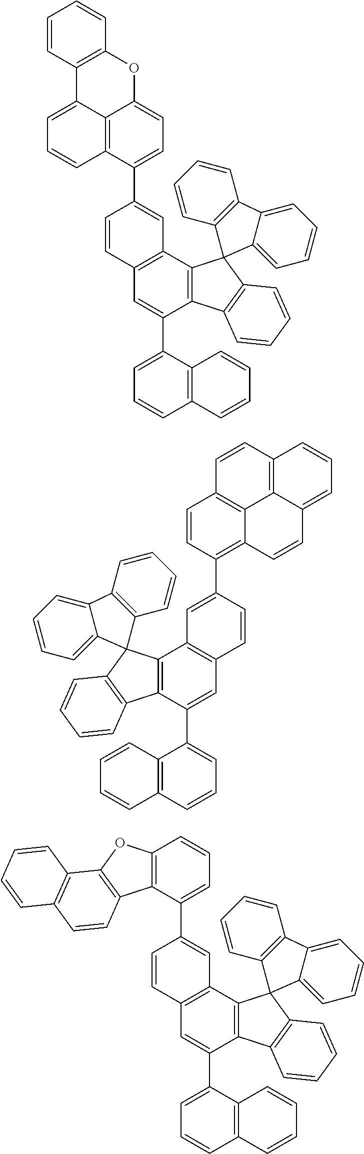

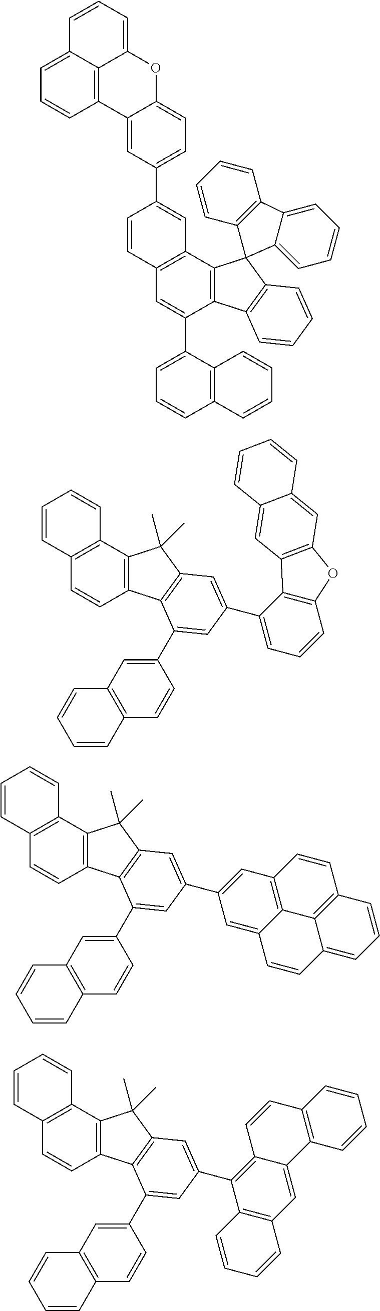

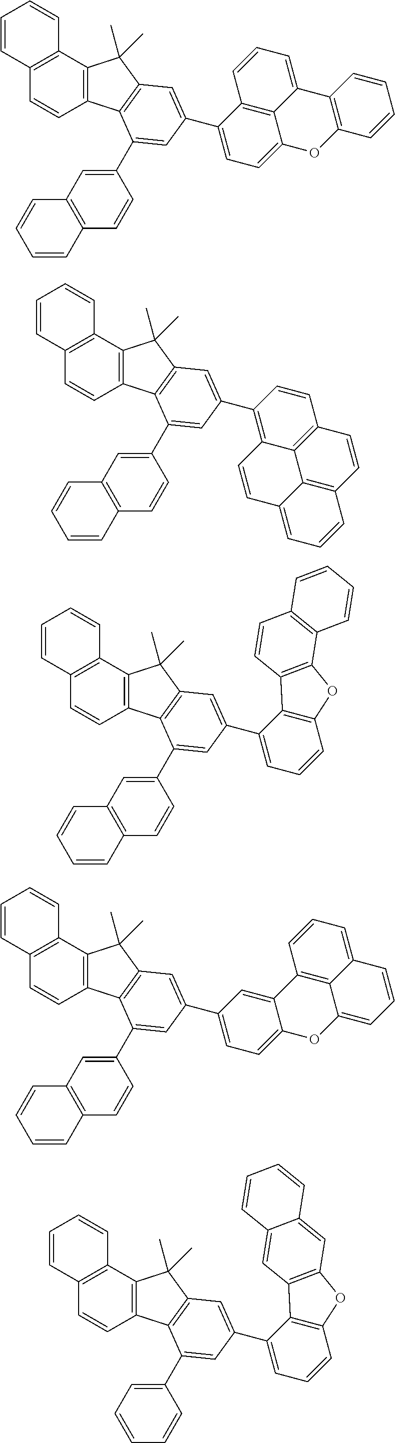

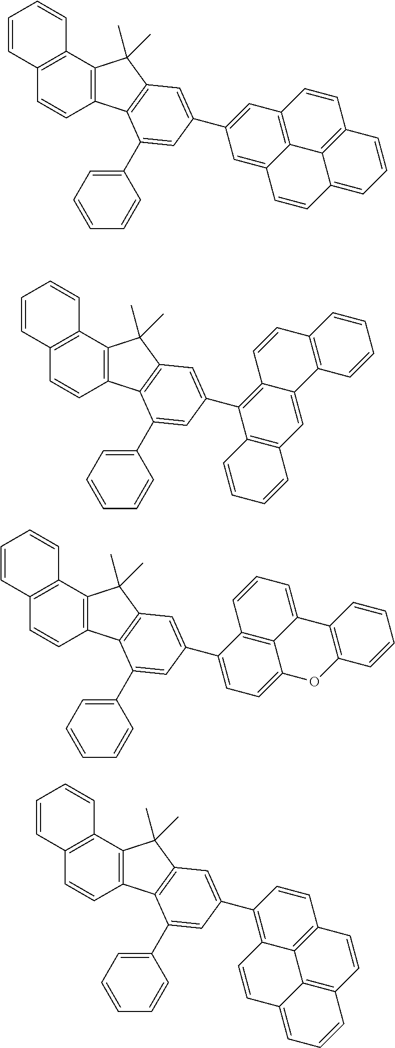

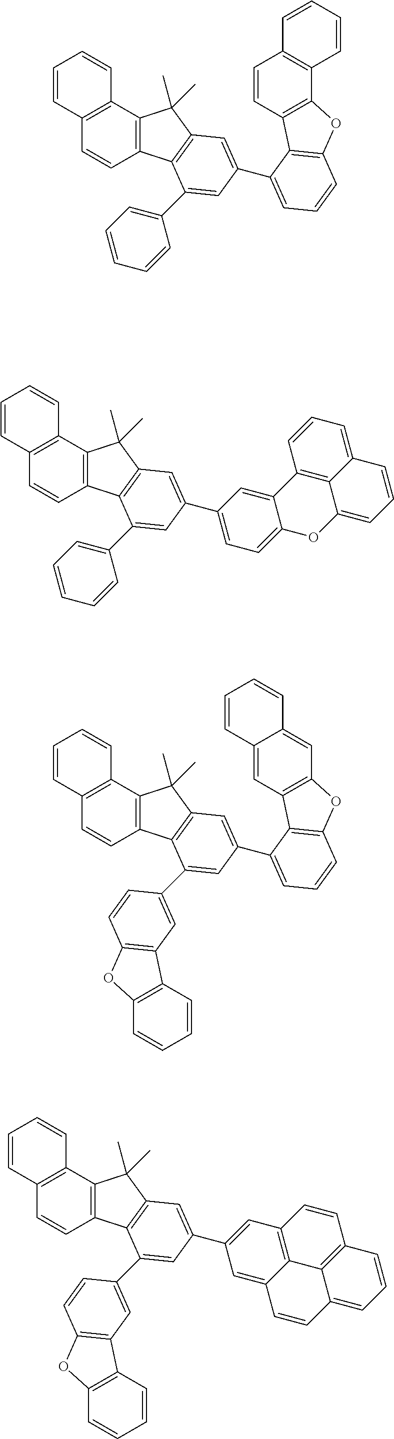 [SMILES: CC1(C)c2cc(-c3cccc4oc5c6ccccc6ccc5c34)cc(-c3ccccc3)c2-c2ccc3ccccc3c21 ; CC1(C)c2cc(-c3ccc4c(c3)-c3cccc5cccc(c35)O4)cc(-c3ccccc3)c2-c2ccc3ccccc3c21 ; CC1(C)c2cc(-c3cccc4oc5cc6ccccc6cc5c34)cc(-c3ccc4oc5ccccc5c4c3)c2-c2ccc3ccccc3c21 ; CC1(C)c2cc(-c3cc4ccc5cccc6ccc(c3)c4c56)cc(-c3ccc4oc5ccccc5c4c3)c2-c2ccc3ccccc3c21]

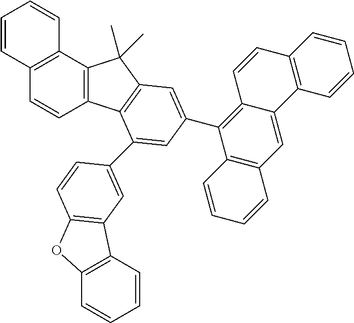 [SMILES: CC1(C)c2cc(-c3c4ccccc4cc4c3ccc3ccccc34)cc(-c3ccc4oc5ccccc5c4c3)c2-c2ccc3ccccc3c21]

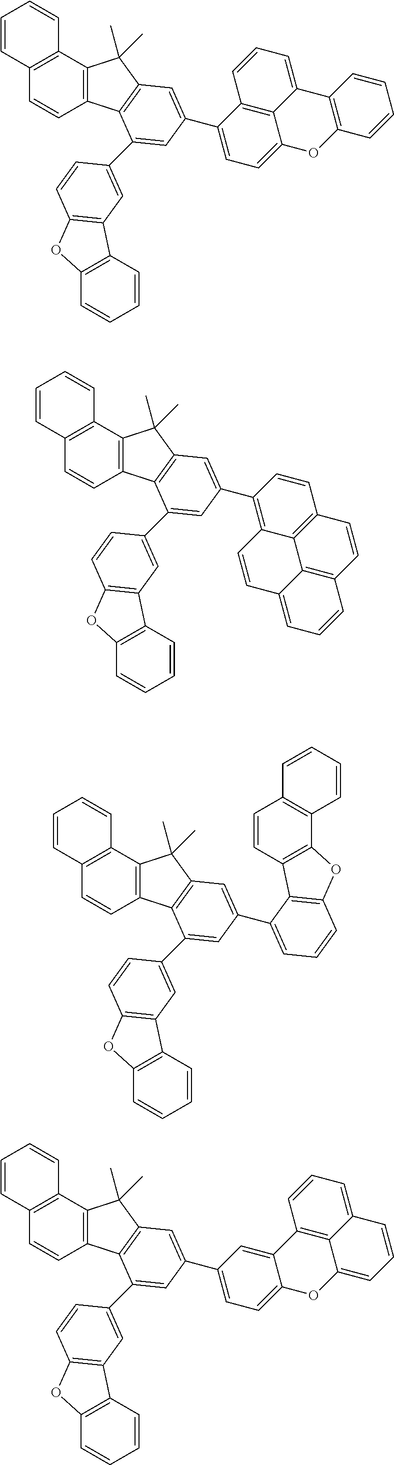 [SMILES: CC1(C)c2cc(-c3ccc4c5c(cccc35)-c3ccccc3O4)cc(-c3ccc4oc5ccccc5c4c3)c2-c2ccc3ccccc3c21 ; CC1(C)c2cc(-c3ccc4ccc5cccc6ccc3c4c56)cc(-c3ccc4oc5ccccc5c4c3)c2-c2ccc3ccccc3c21 ; CC1(C)c2cc(-c3cccc4oc5c6ccccc6ccc5c34)cc(-c3ccc4oc5ccccc5c4c3)c2-c2ccc3ccccc3c21 ; CC1(C)c2cc(-c3ccc4c(c3)-c3cccc5cccc(c35)O4)cc(-c3ccc4oc5ccccc5c4c3)c2-c2ccc3ccccc3c21]

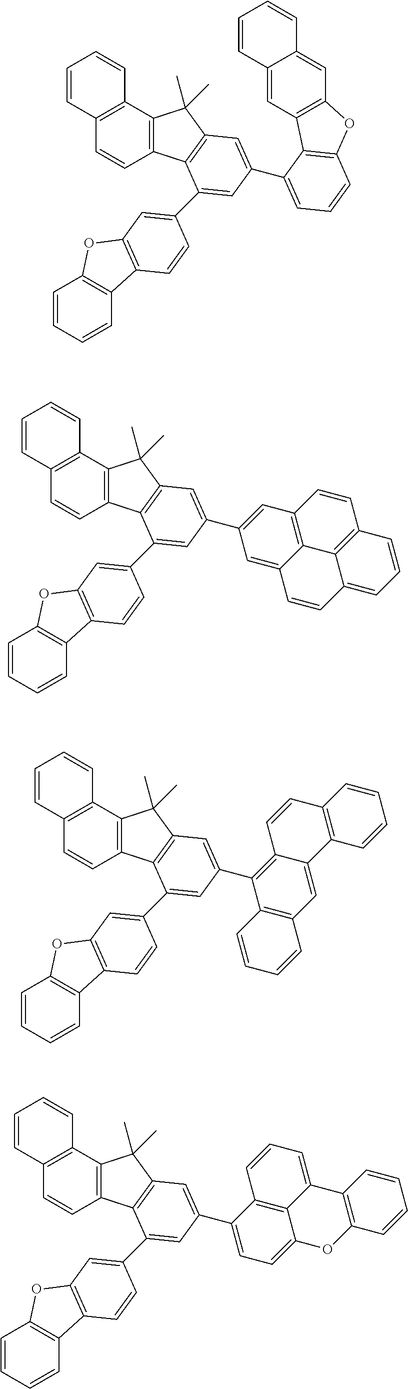 [SMILES: CC1(C)c2cc(-c3cccc4oc5cc6ccccc6cc5c34)cc(-c3ccc4c(c3)oc3ccccc34)c2-c2ccc3ccccc3c21 ; CC1(C)c2cc(-c3cc4ccc5cccc6ccc(c3)c4c56)cc(-c3ccc4c(c3)oc3ccccc34)c2-c2ccc3ccccc3c21 ; CC1(C)c2cc(-c3c4ccccc4cc4c3ccc3ccccc34)cc(-c3ccc4c(c3)oc3ccccc34)c2-c2ccc3ccccc3c21 ; CC1(C)c2cc(-c3ccc4c5c(cccc35)-c3ccccc3O4)cc(-c3ccc4c(c3)oc3ccccc34)c2-c2ccc3ccccc3c21]

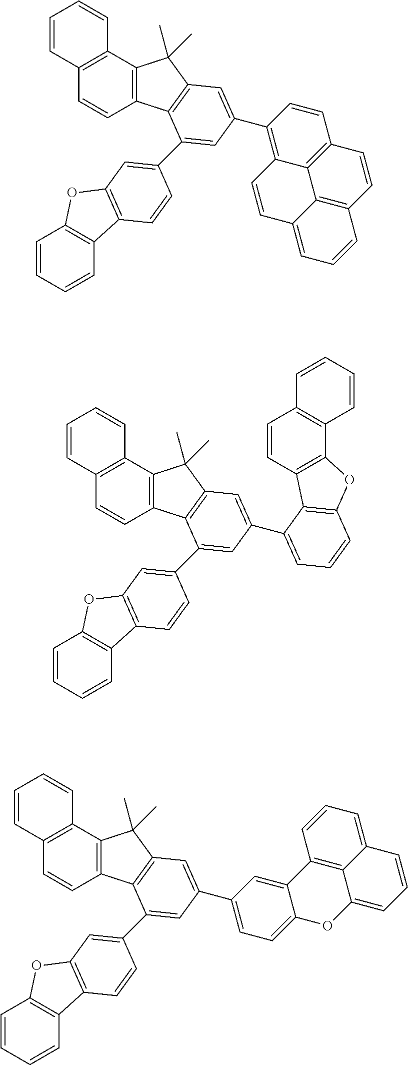 [SMILES: CC1(C)c2cc(-c3ccc4ccc5cccc6ccc3c4c56)cc(-c3ccc4c(c3)oc3ccccc34)c2-c2ccc3ccccc3c21 ; CC1(C)c2cc(-c3cccc4oc5c6ccccc6ccc5c34)cc(-c3ccc4c(c3)oc3ccccc34)c2-c2ccc3ccccc3c21 ; CC1(C)c2cc(-c3ccc4c(c3)-c3cccc5cccc(c35)O4)cc(-c3ccc4c(c3)oc3ccccc34)c2-c2ccc3ccccc3c21]

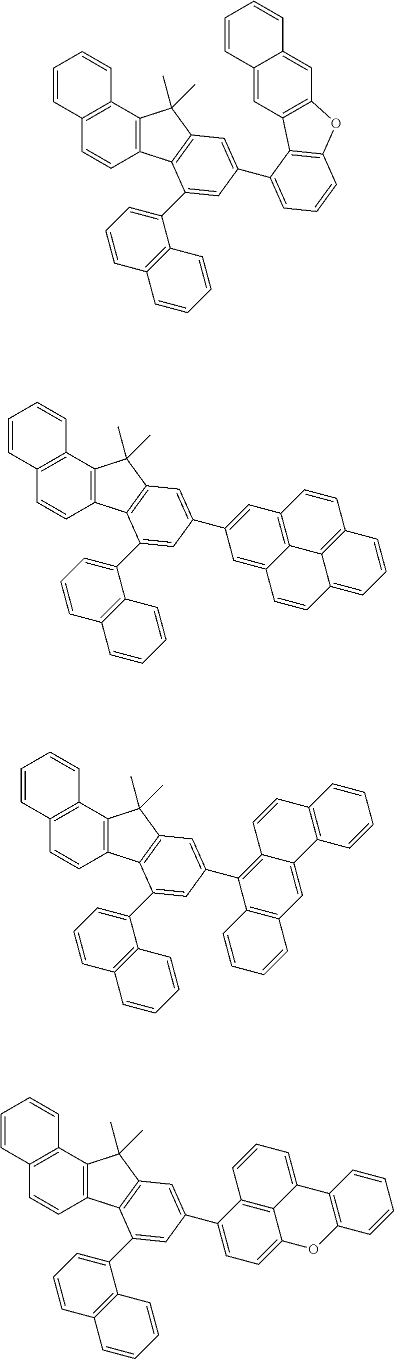

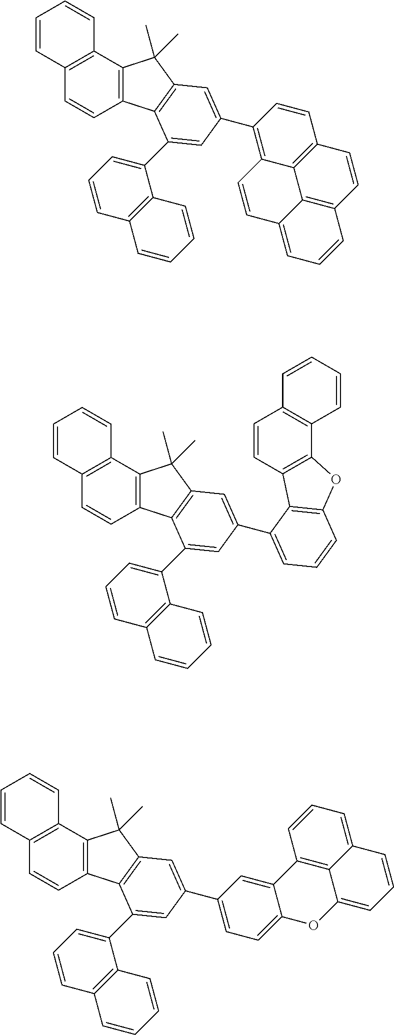

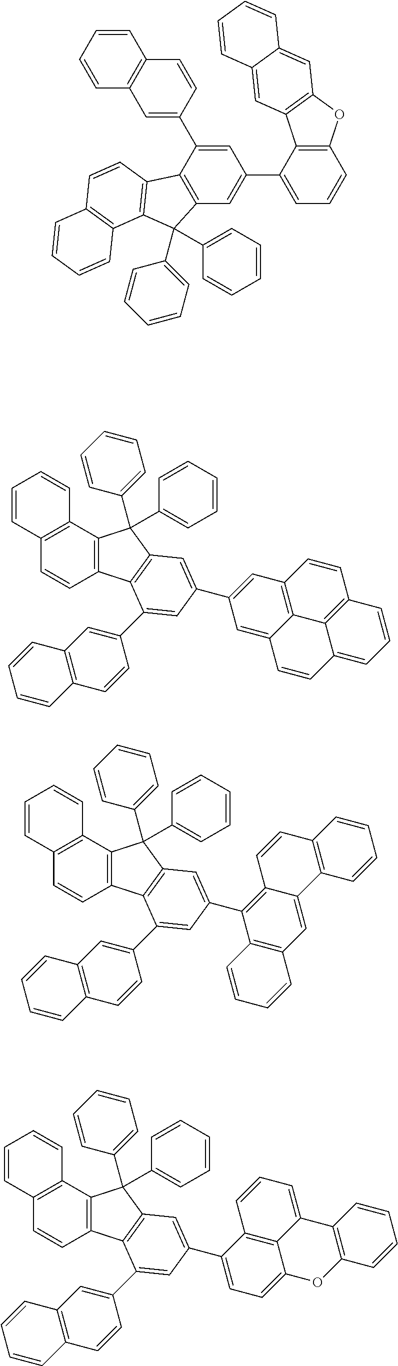

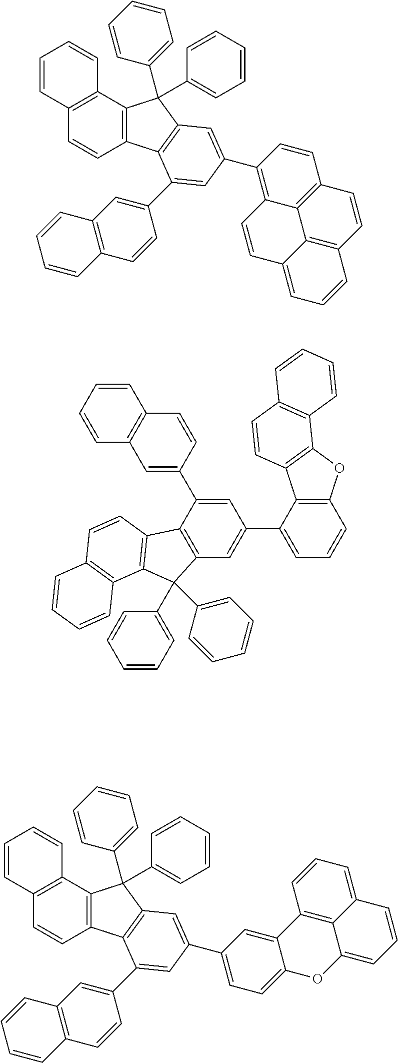

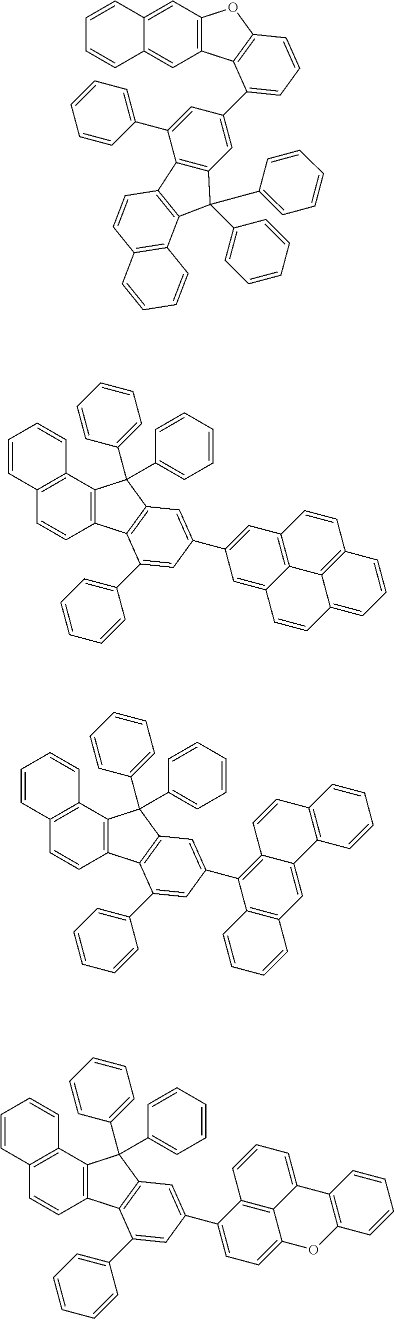

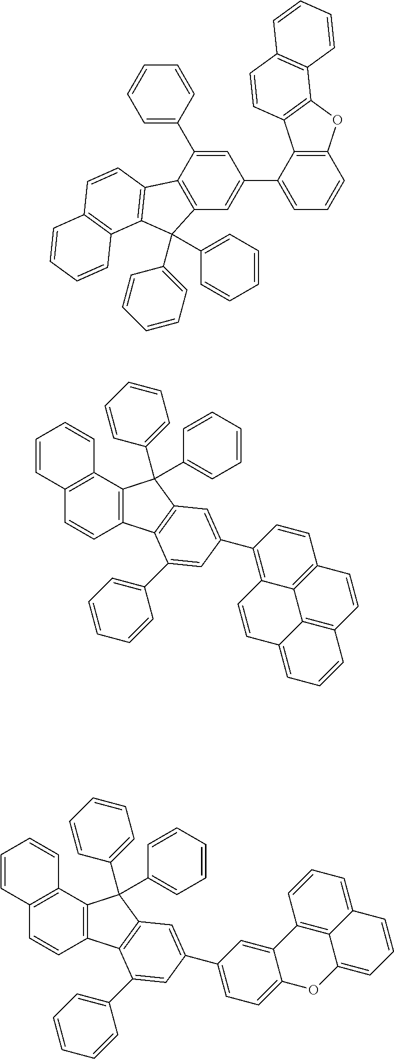

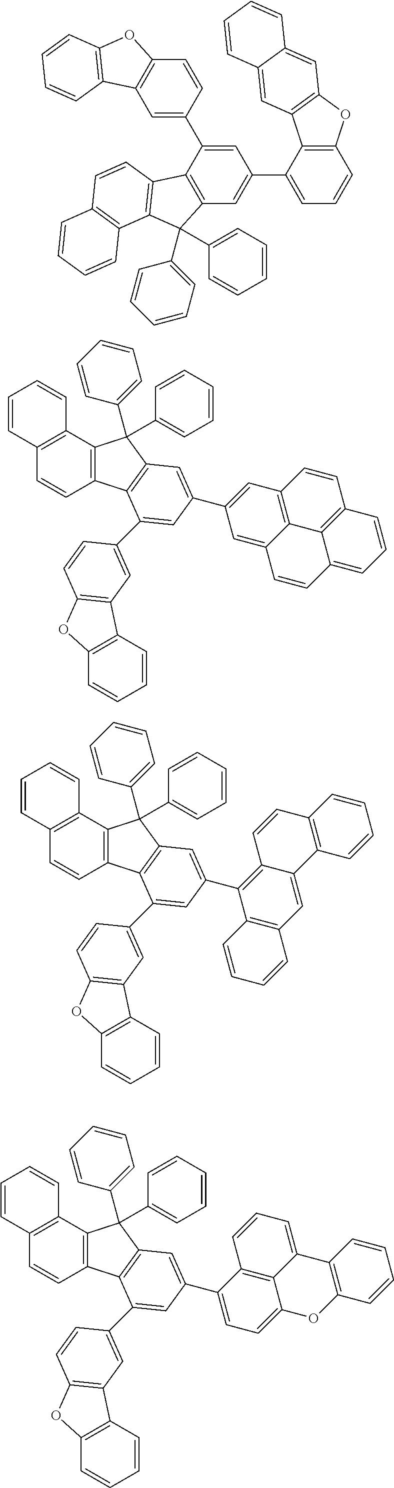

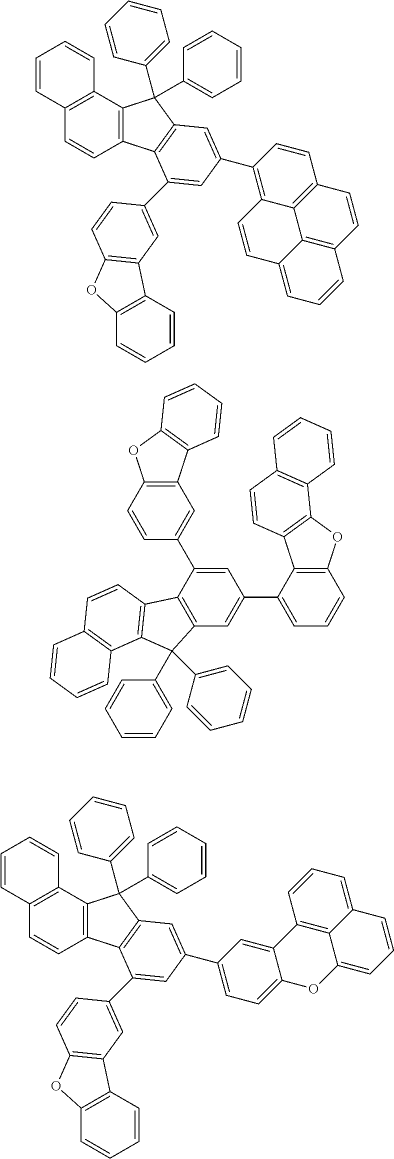

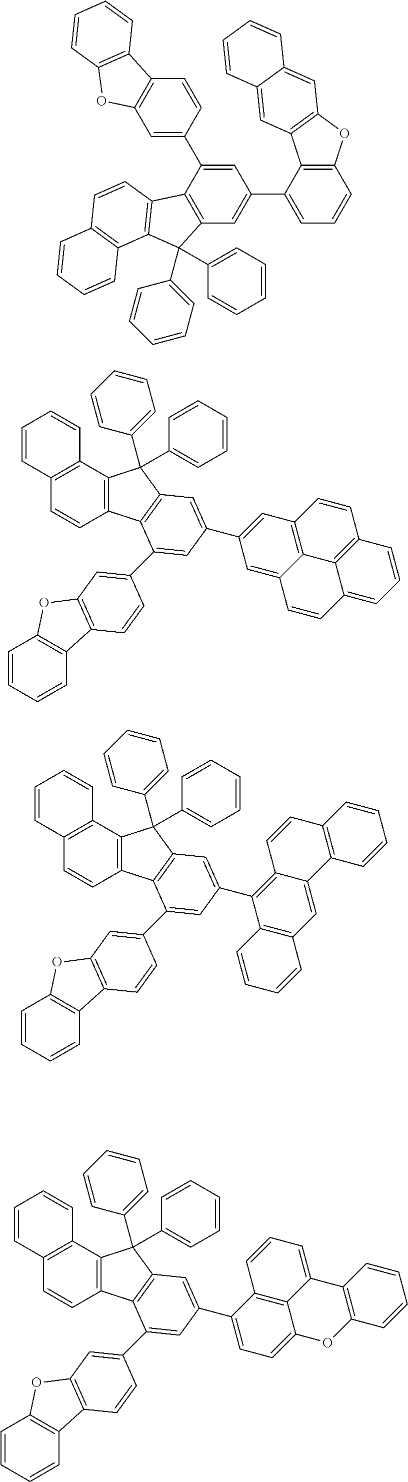

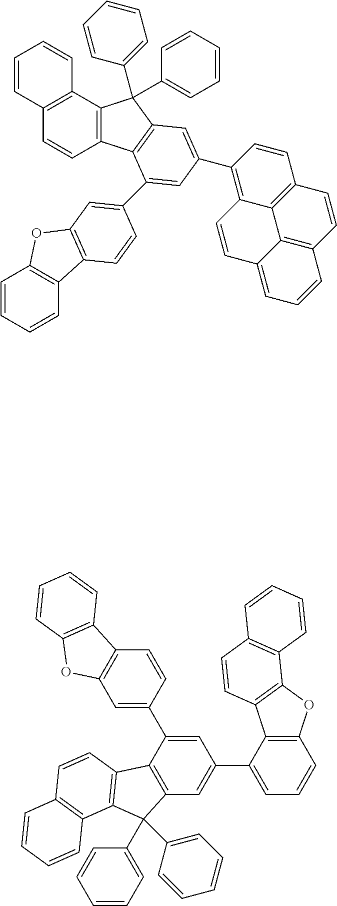 [SMILES: c1ccc(C2(c3ccccc3)c3cc(-c4ccc5ccc6cccc7ccc4c5c67)cc(-c4ccc5c(c4)oc4ccccc45)c3-c3ccc4ccccc4c32)cc1 ; c1ccc(C2(c3ccccc3)c3cc(-c4cccc5oc6c7ccccc7ccc6c45)cc(-c4ccc5c(c4)oc4ccccc45)c3-c3ccc4ccccc4c32)cc1]

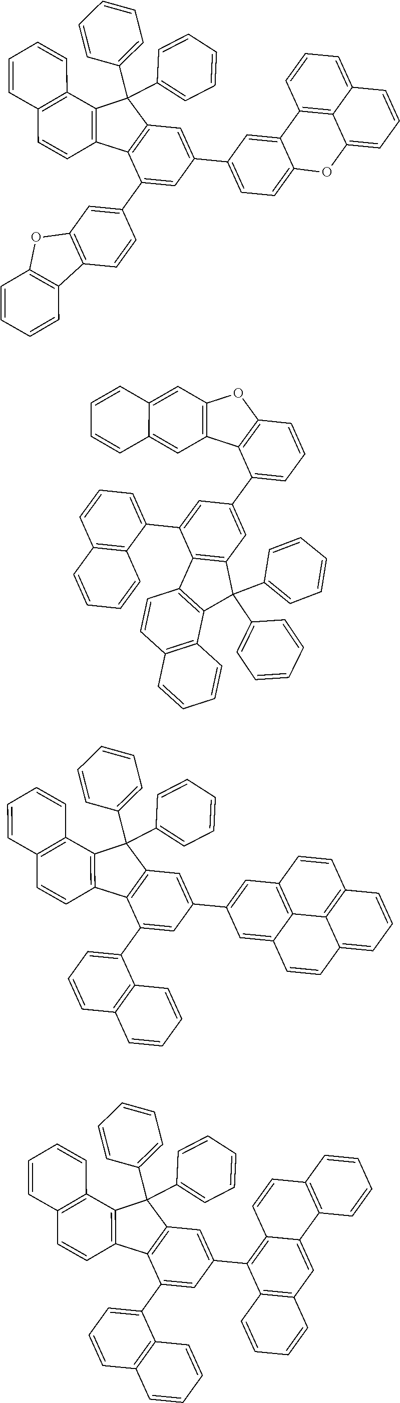

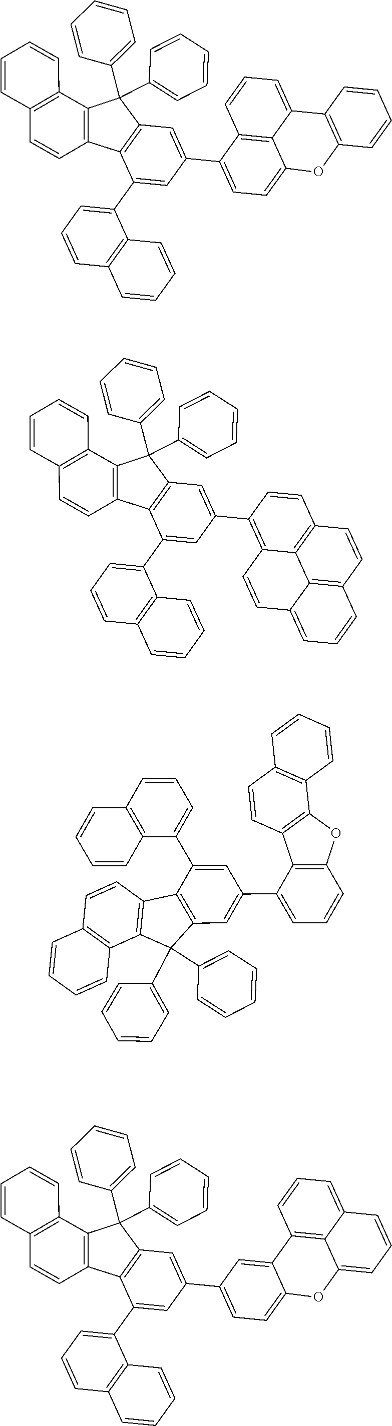

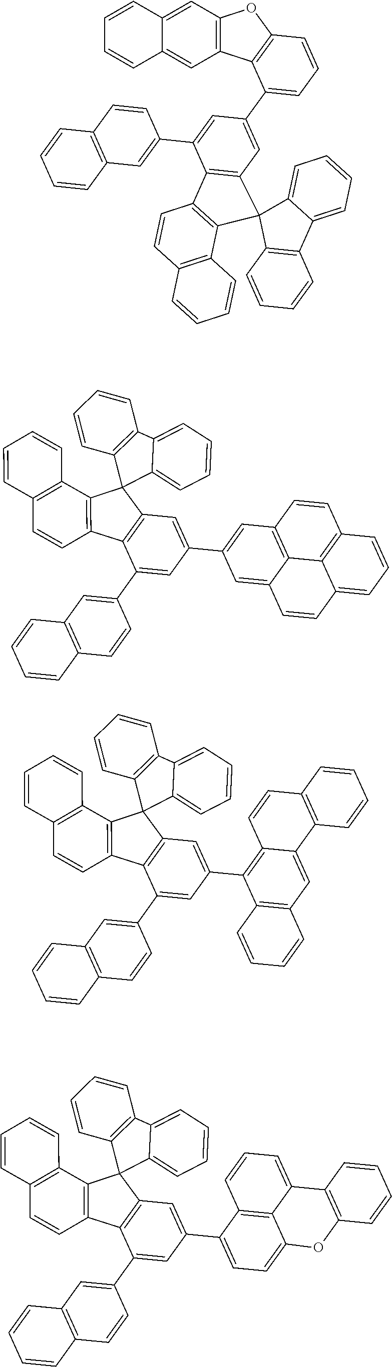

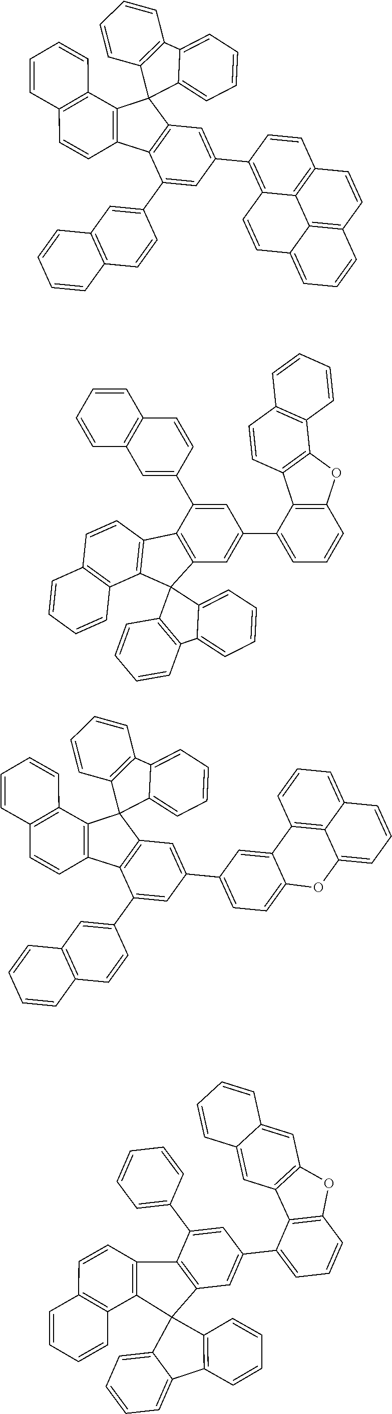

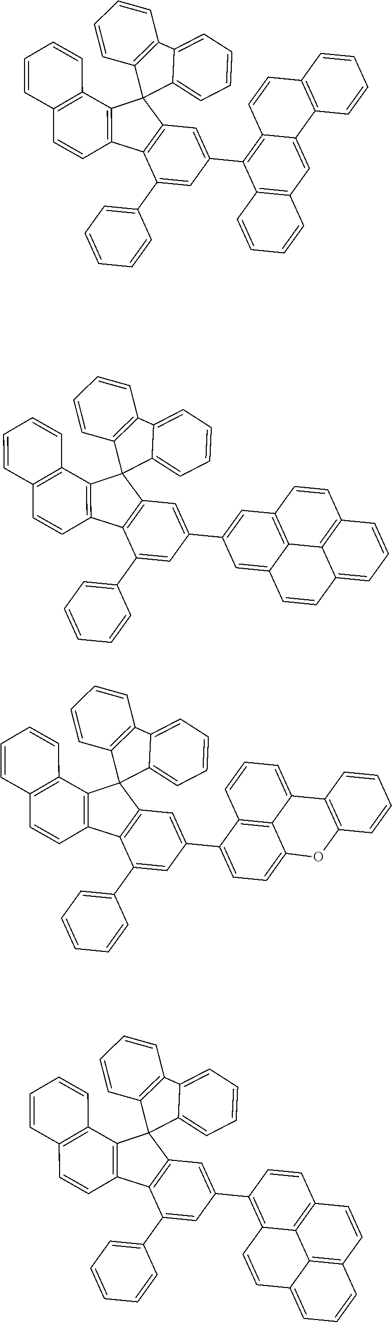

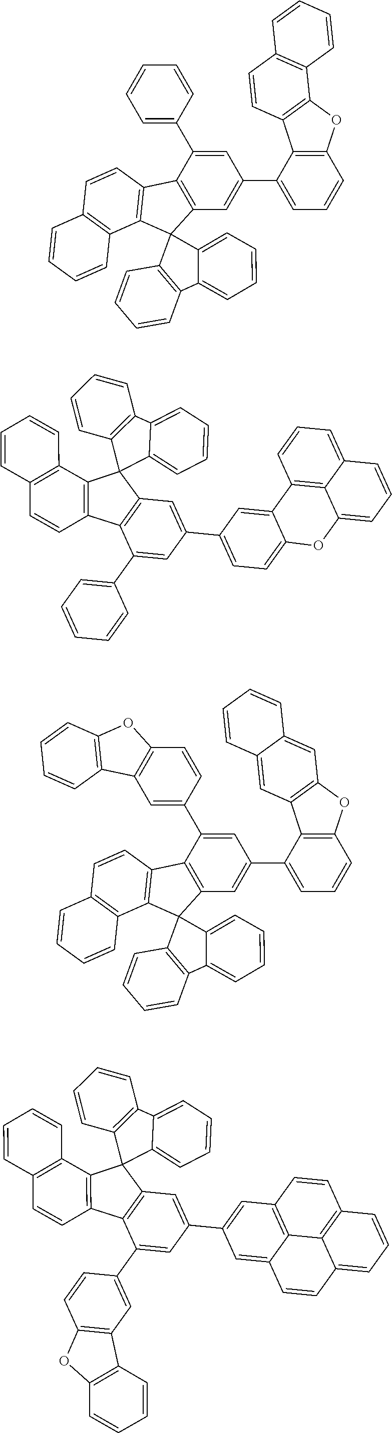 [SMILES: c1ccc(-c2cc(-c3cccc4oc5c6ccccc6ccc5c34)cc3c2-c2ccc4ccccc4c2C32c3ccccc3-c3ccccc32)cc1 ; c1ccc(-c2cc(-c3ccc4c(c3)-c3cccc5cccc(c35)O4)cc3c2-c2ccc4ccccc4c2C32c3ccccc3-c3ccccc32)cc1 ; c1ccc2c(c1)-c1ccccc1C21c2cc(-c3cccc4oc5cc6ccccc6cc5c34)cc(-c3ccc4oc5ccccc5c4c3)c2-c2ccc3ccccc3c21 ; c1ccc2c(c1)-c1ccccc1C21c2cc(-c3cc4ccc5cccc6ccc(c3)c4c56)cc(-c3ccc4oc5ccccc5c4c3)c2-c2ccc3ccccc3c21]

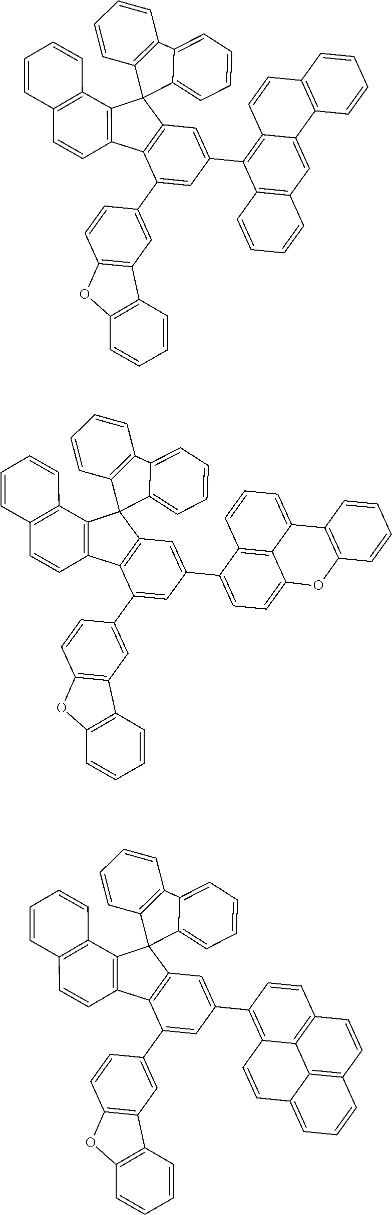 [SMILES: c1ccc2c(c1)-c1ccccc1C21c2cc(-c3c4ccccc4cc4c3ccc3ccccc34)cc(-c3ccc4oc5ccccc5c4c3)c2-c2ccc3ccccc3c21 ; c1ccc2c(c1)Oc1ccc(-c3cc(-c4ccc5oc6ccccc6c5c4)c4c(c3)C3(c5ccccc5-c5ccccc53)c3c-4ccc4ccccc34)c3cccc-2c13 ; c1ccc2c(c1)-c1ccccc1C21c2cc(-c3ccc4ccc5cccc6ccc3c4c56)cc(-c3ccc4oc5ccccc5c4c3)c2-c2ccc3ccccc3c21]

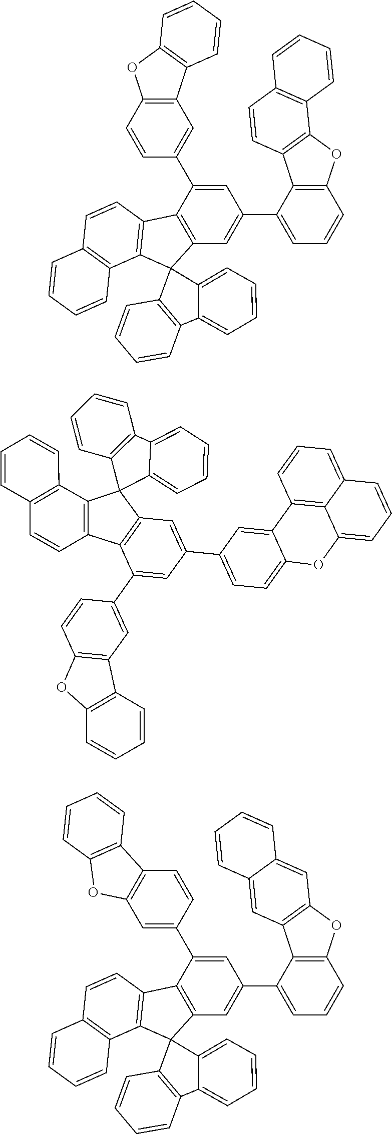

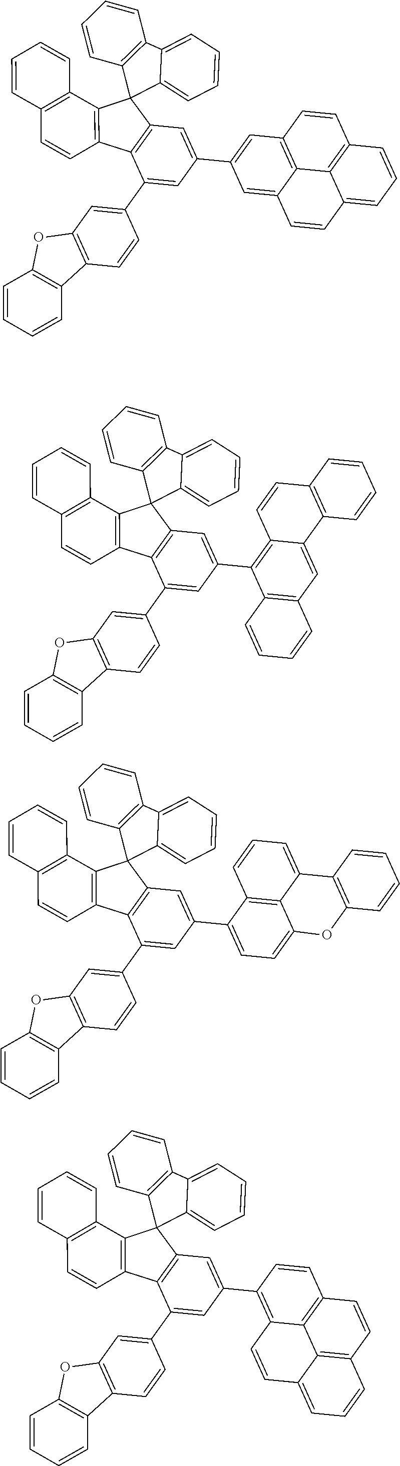

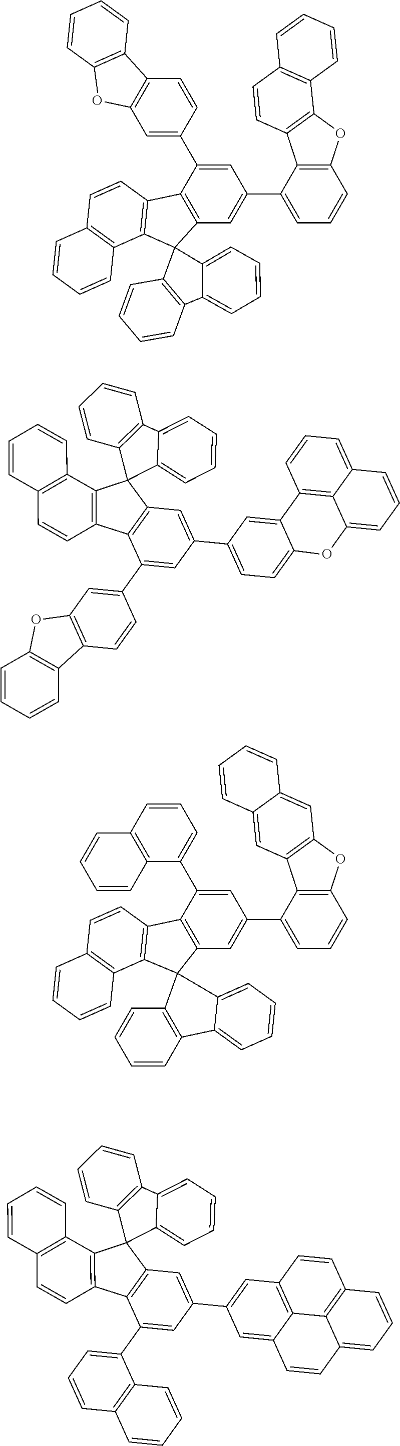

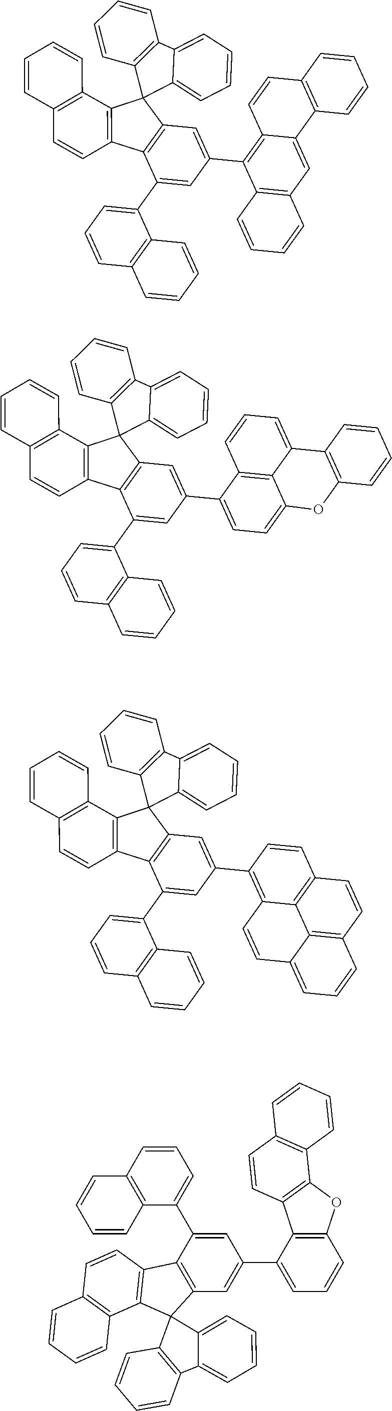

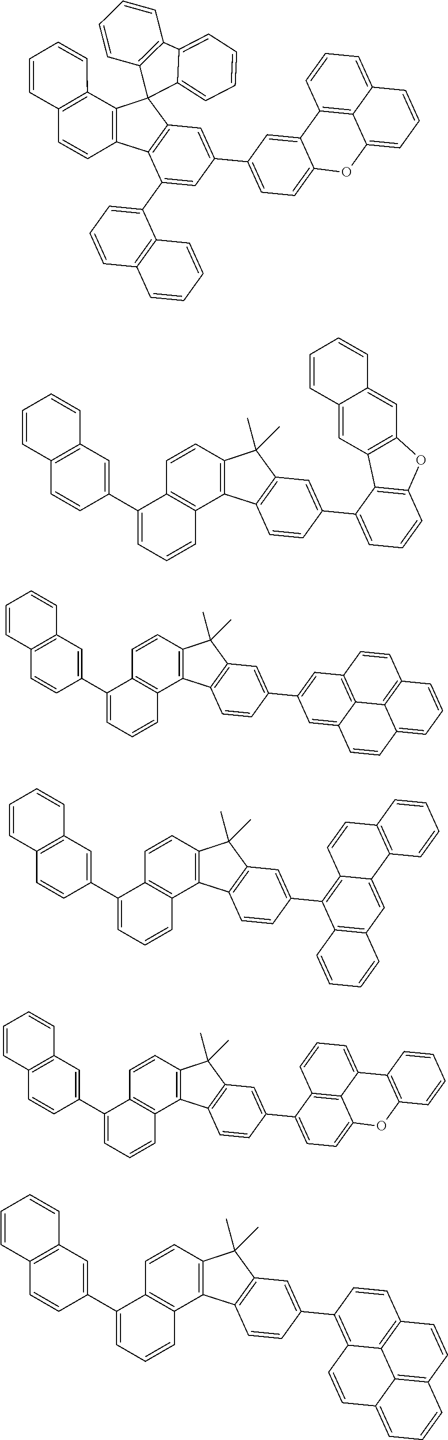

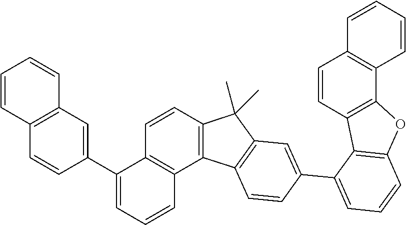 [SMILES: CC1(C)c2cc(-c3cccc4oc5c6ccccc6ccc5c34)ccc2-c2c1ccc1c(-c3ccc4ccccc4c3)cccc21]

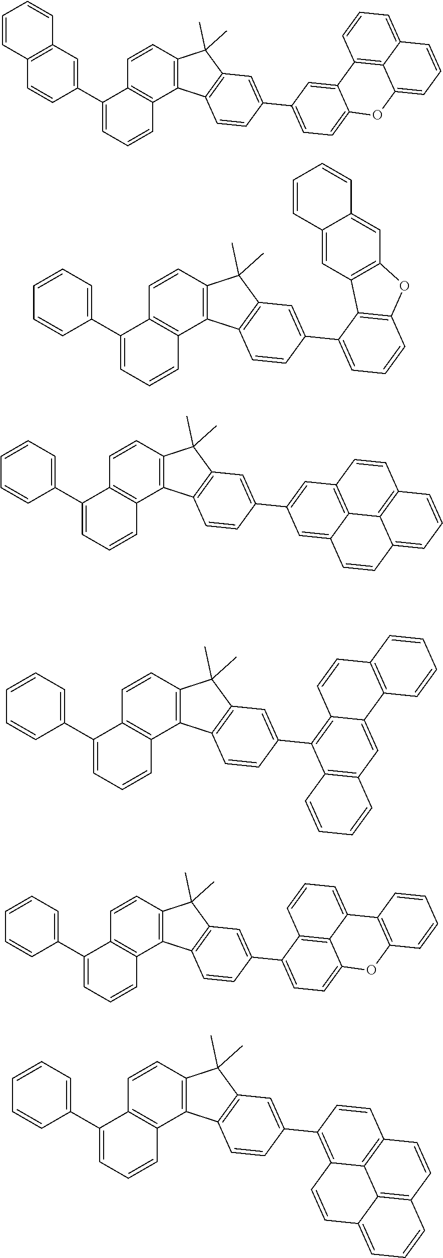

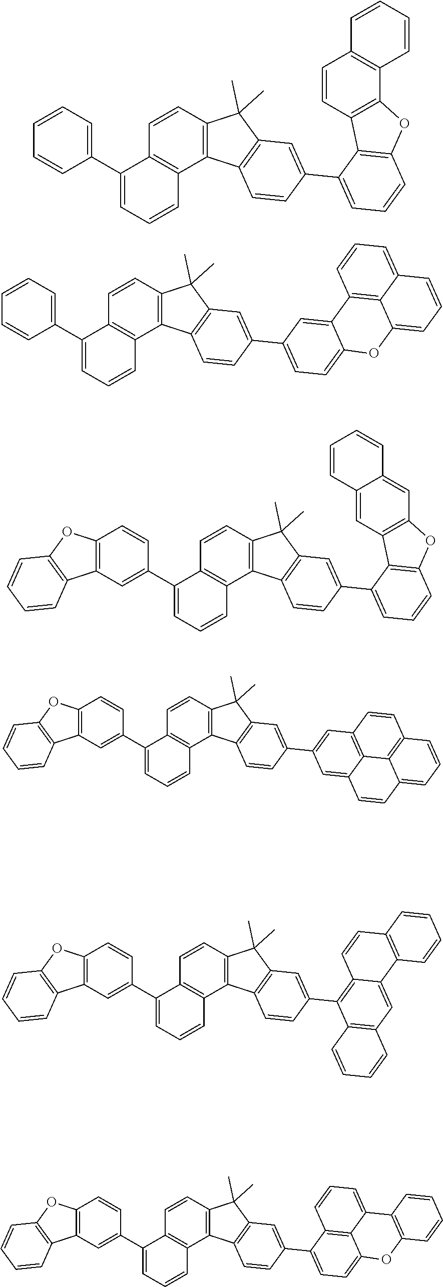

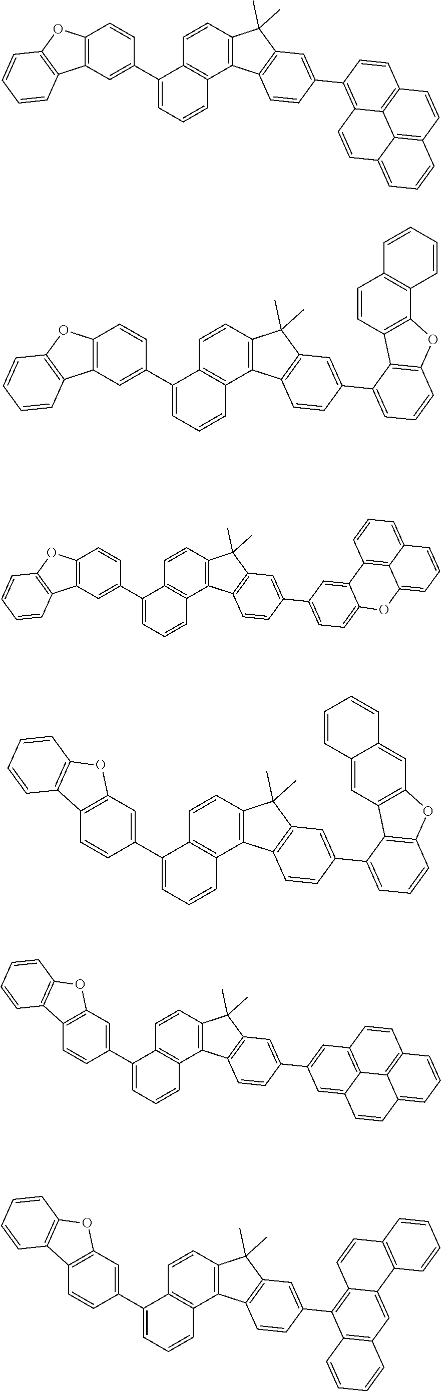

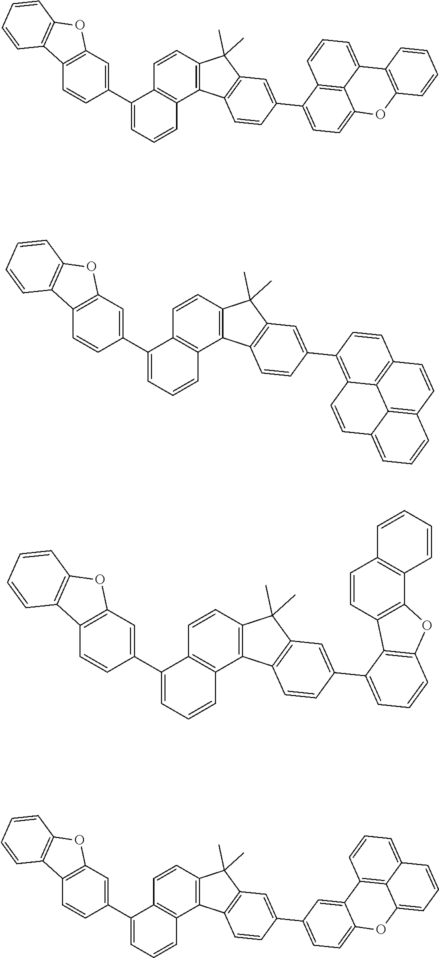 [SMILES: CC1(C)c2cc(-c3ccc4c5c(cccc35)-c3ccccc3O4)ccc2-c2c1ccc1c(-c3ccc4c(c3)oc3ccccc34)cccc21 ; CC1(C)c2cc(-c3ccc4ccc5cccc6ccc3c4c56)ccc2-c2c1ccc1c(-c3ccc4c(c3)oc3ccccc34)cccc21 ; CC1(C)c2cc(-c3cccc4oc5c6ccccc6ccc5c34)ccc2-c2c1ccc1c(-c3ccc4c(c3)oc3ccccc34)cccc21 ; CC1(C)c2cc(-c3ccc4c(c3)-c3cccc5cccc(c35)O4)ccc2-c2c1ccc1c(-c3ccc4c(c3)oc3ccccc34)cccc21]

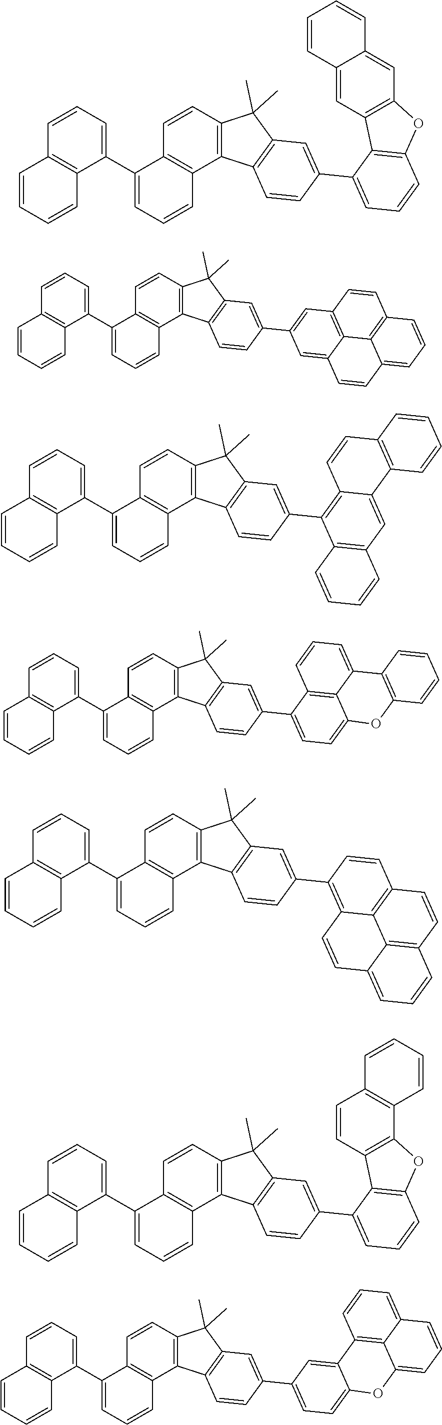

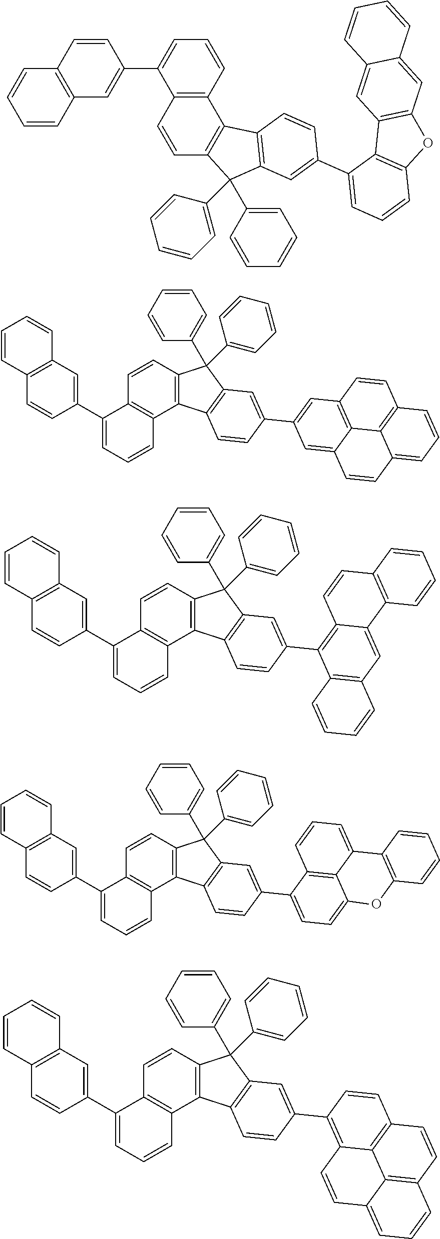

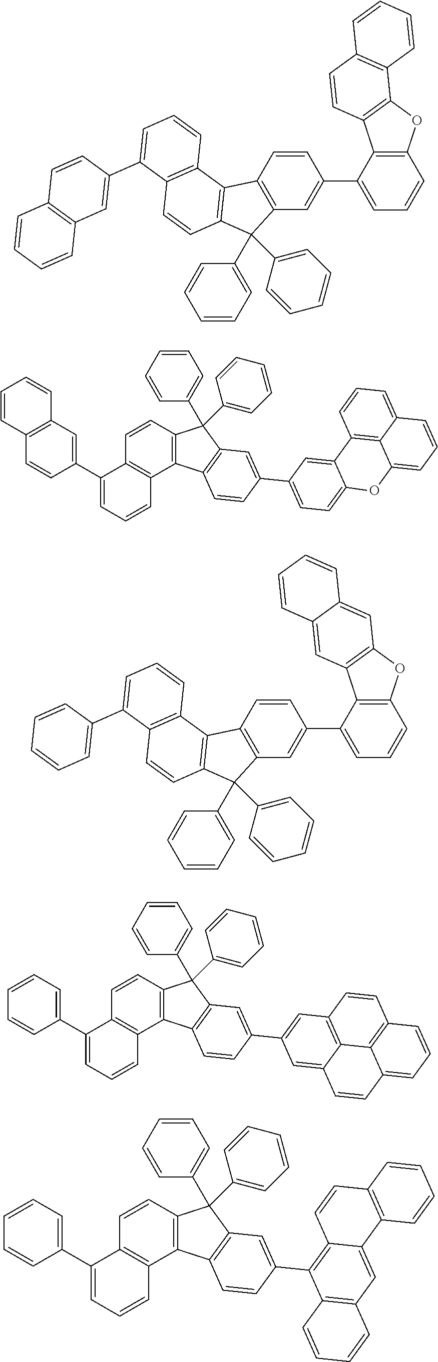

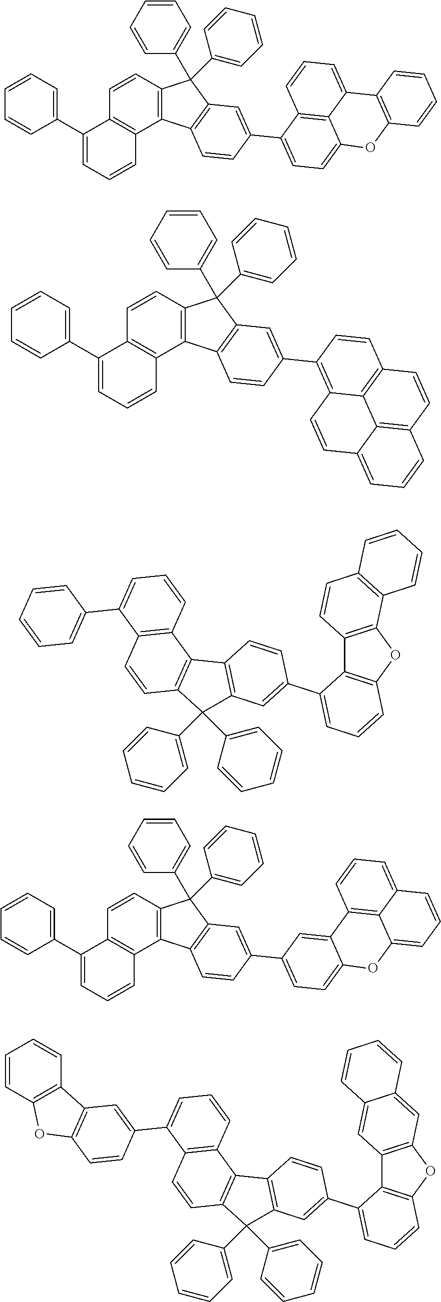

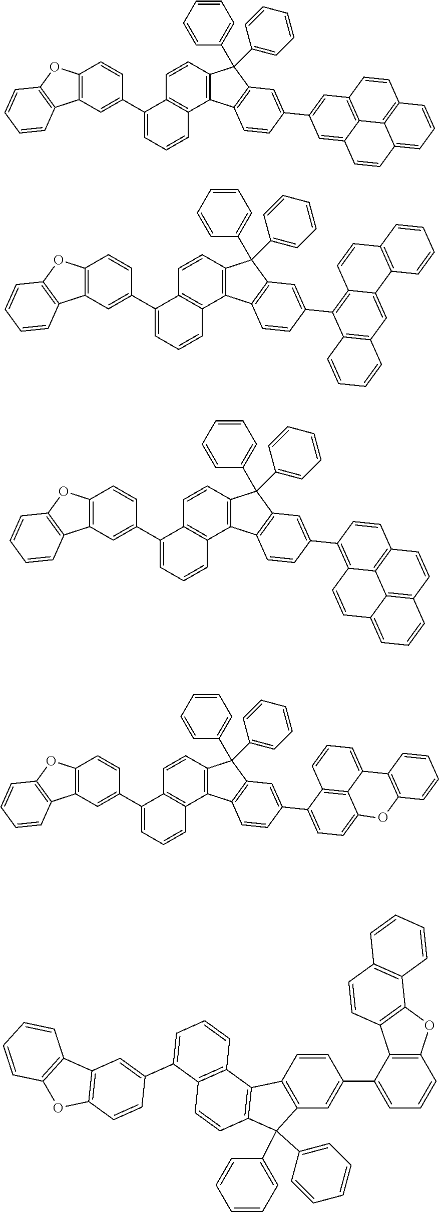 [SMILES: c1ccc(C2(c3ccccc3)c3cc(-c4cc5ccc6cccc7ccc(c4)c5c67)ccc3-c3c2ccc2c(-c4ccc5oc6ccccc6c5c4)cccc32)cc1 ; c1ccc(C2(c3ccccc3)c3cc(-c4c5ccccc5cc5c4ccc4ccccc45)ccc3-c3c2ccc2c(-c4ccc5oc6ccccc6c5c4)cccc32)cc1 ; c1ccc(C2(c3ccccc3)c3cc(-c4ccc5ccc6cccc7ccc4c5c67)ccc3-c3c2ccc2c(-c4ccc5oc6ccccc6c5c4)cccc32)cc1 ; c1ccc(C2(c3ccccc3)c3cc(-c4ccc5c6c(cccc46)-c4ccccc4O5)ccc3-c3c2ccc2c(-c4ccc5oc6ccccc6c5c4)cccc32)cc1 ; c1ccc(C2(c3ccccc3)c3cc(-c4cccc5oc6c7ccccc7ccc6c45)ccc3-c3c2ccc2c(-c4ccc5oc6ccccc6c5c4)cccc32)cc1]

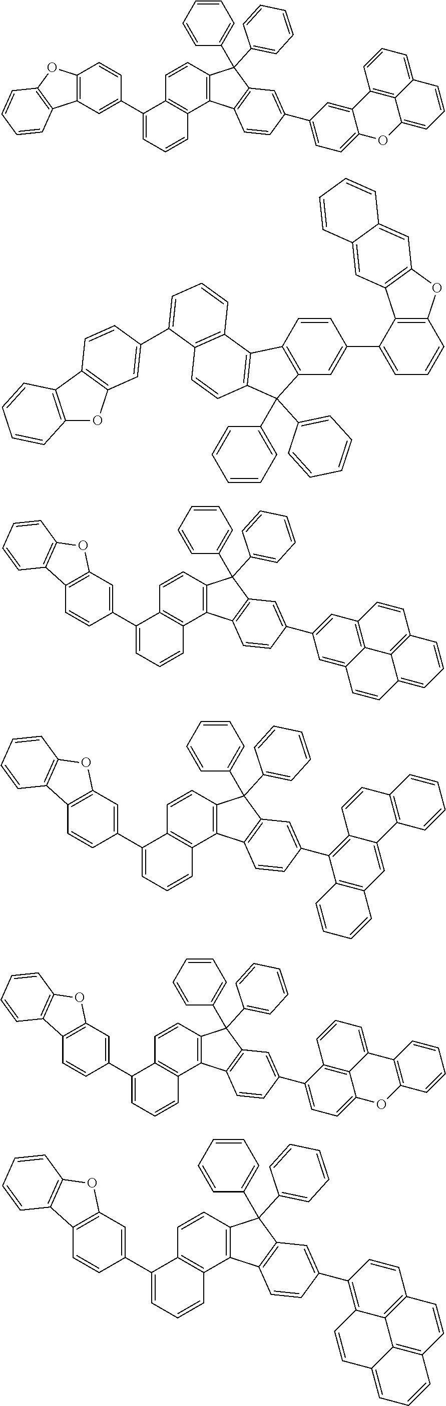

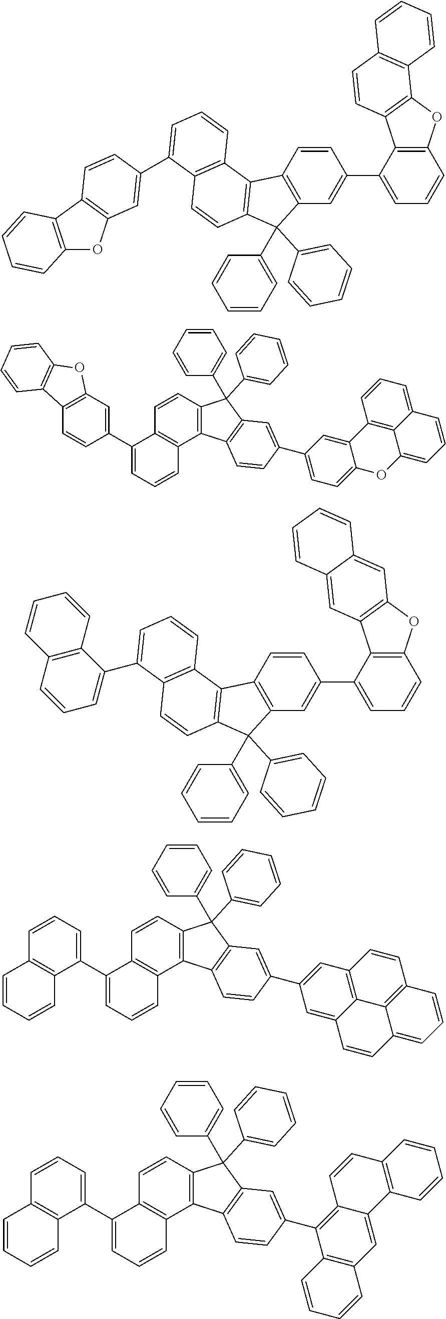

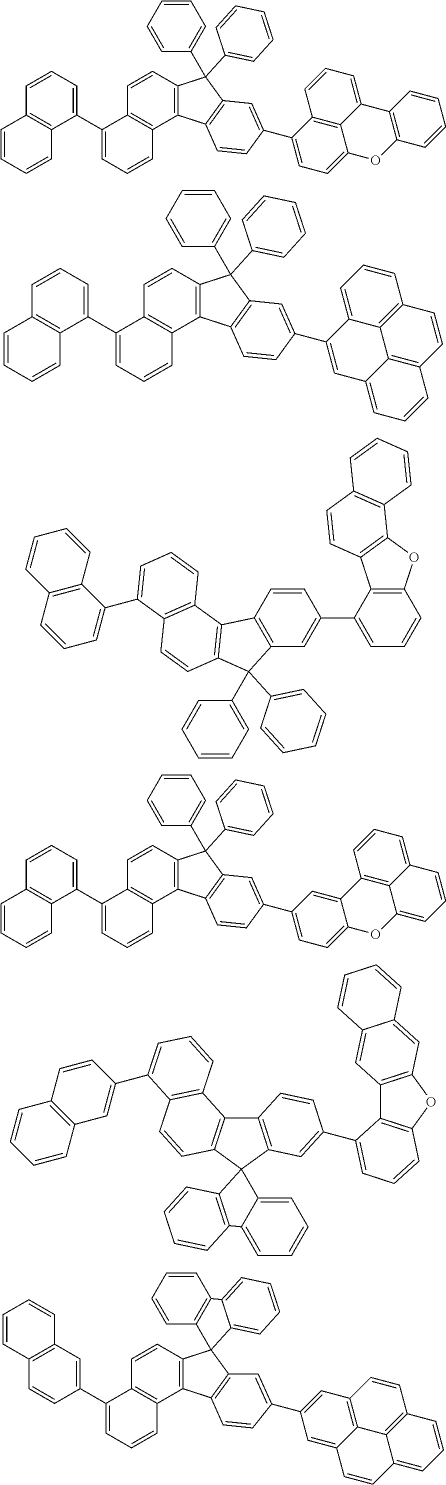

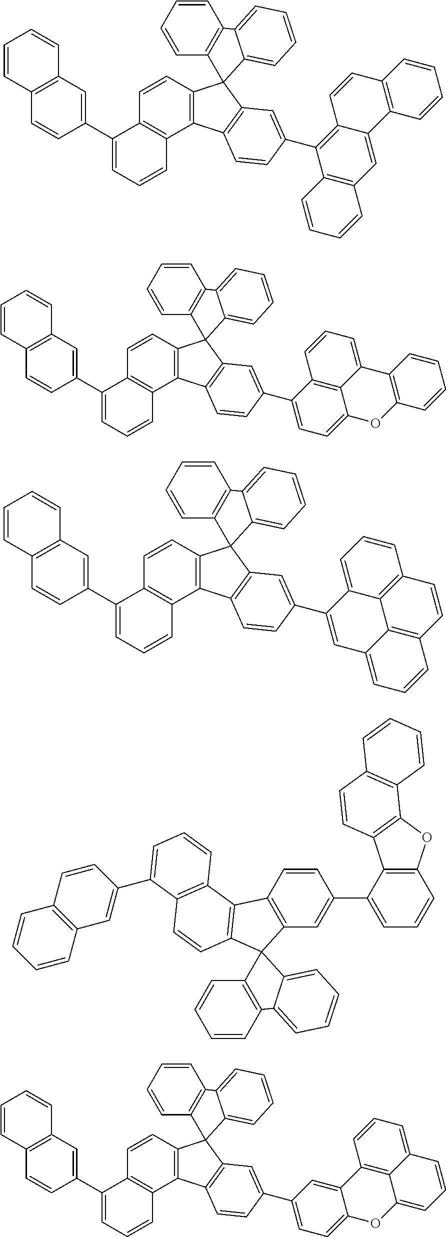

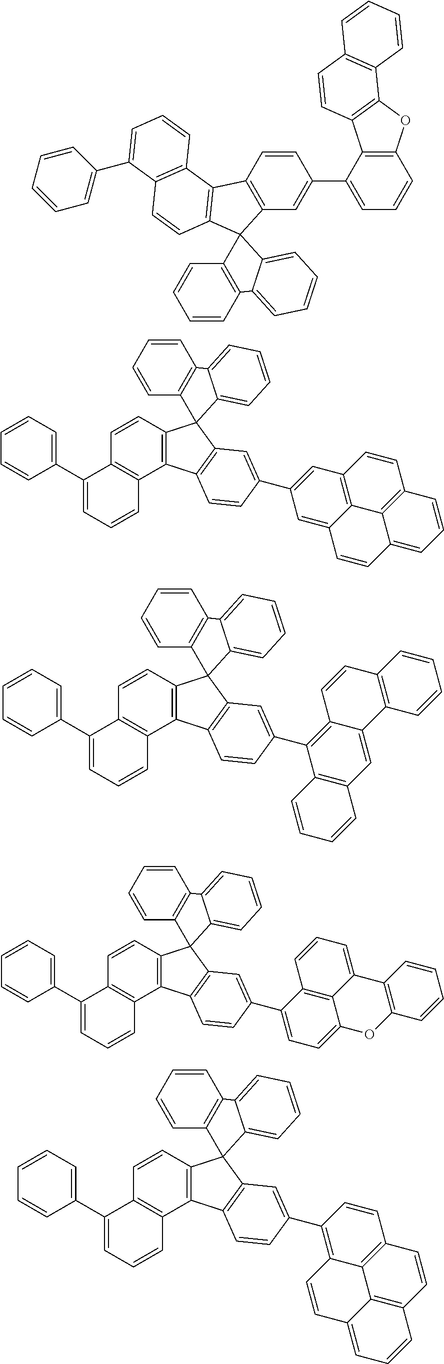

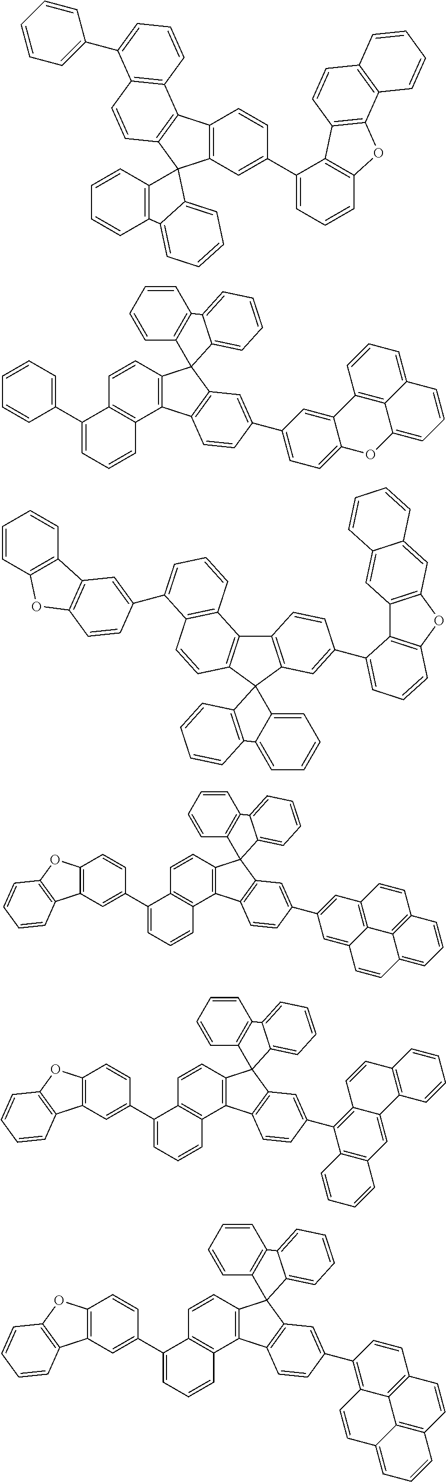

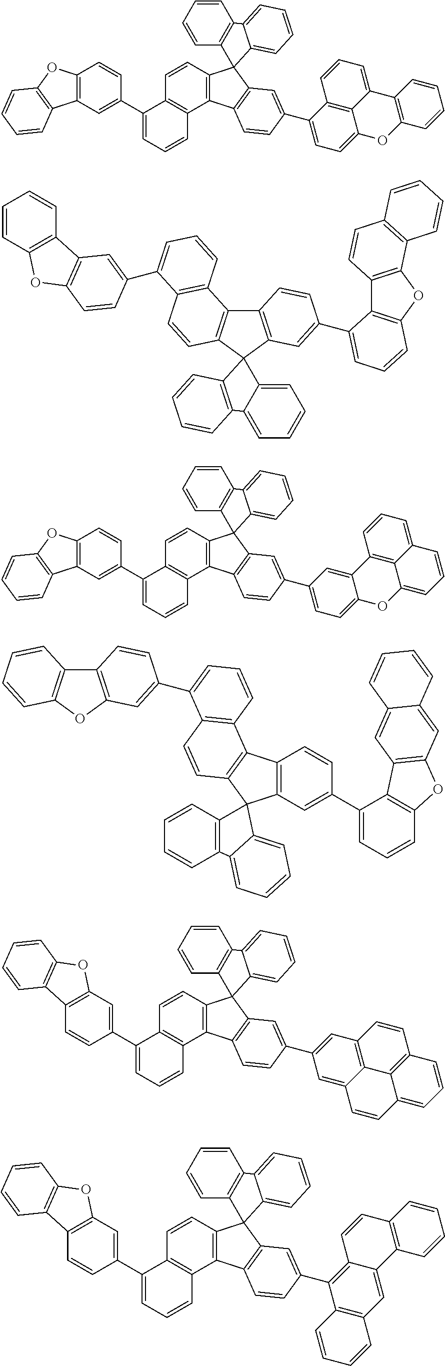

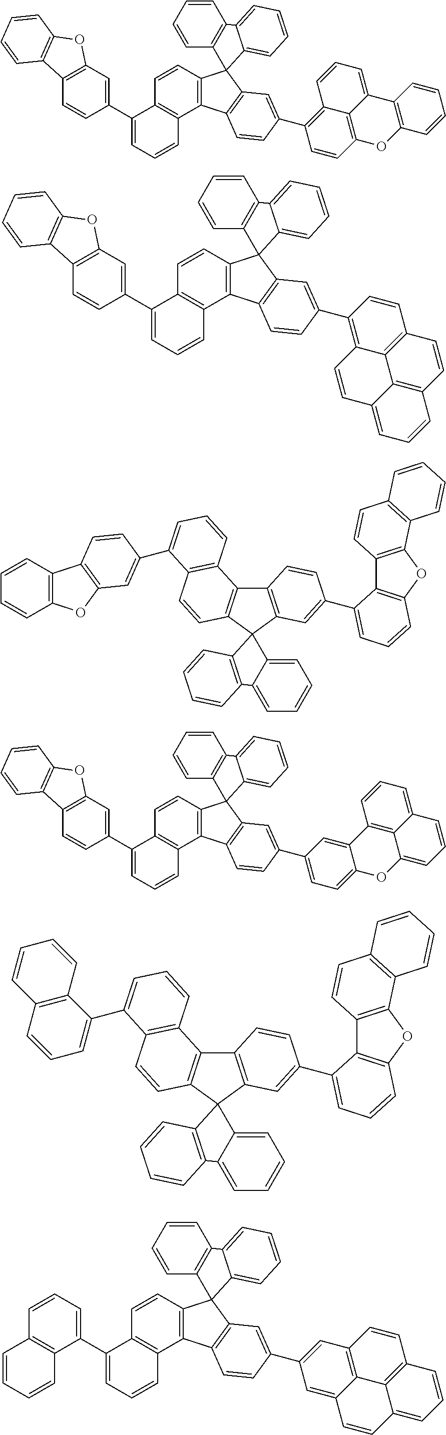

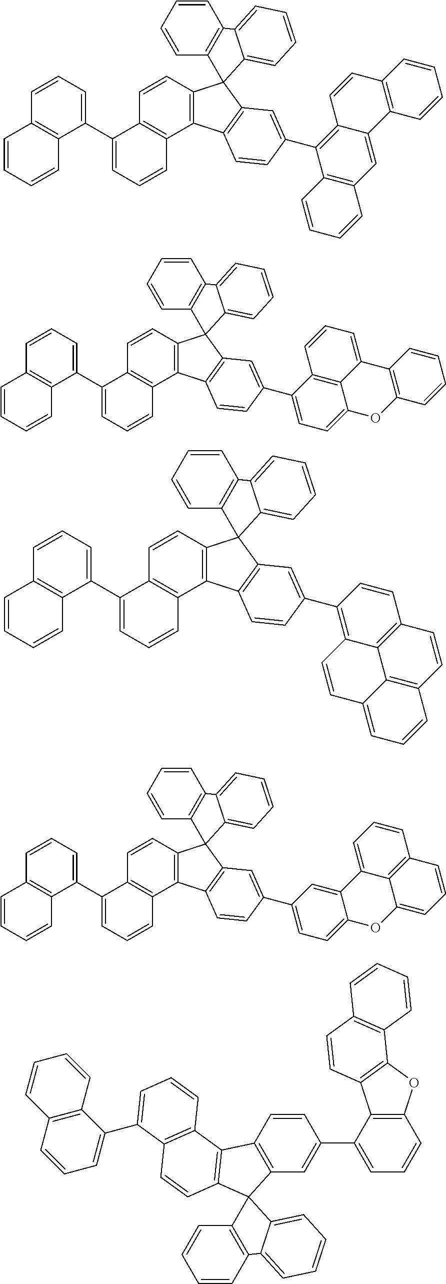

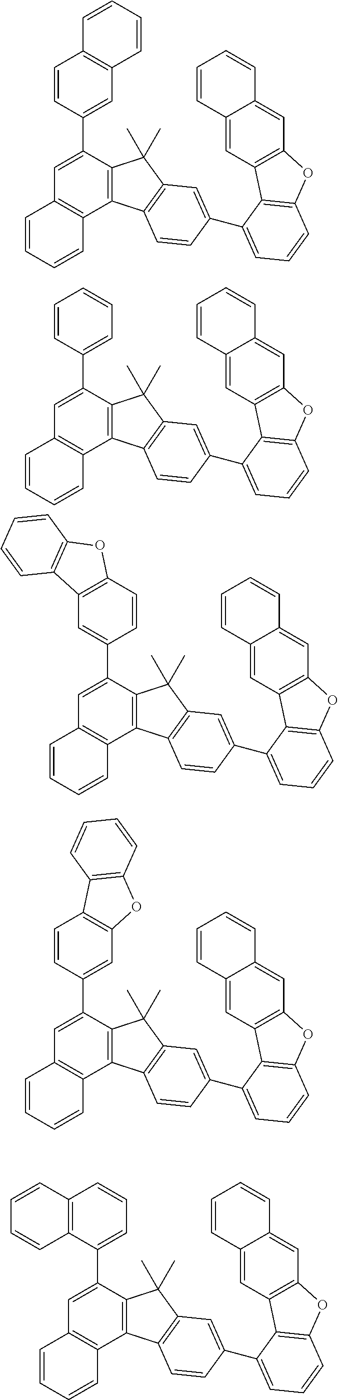

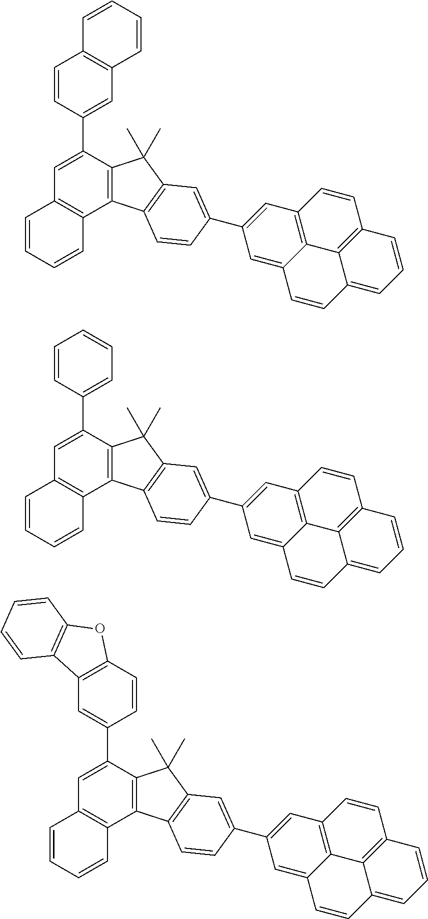

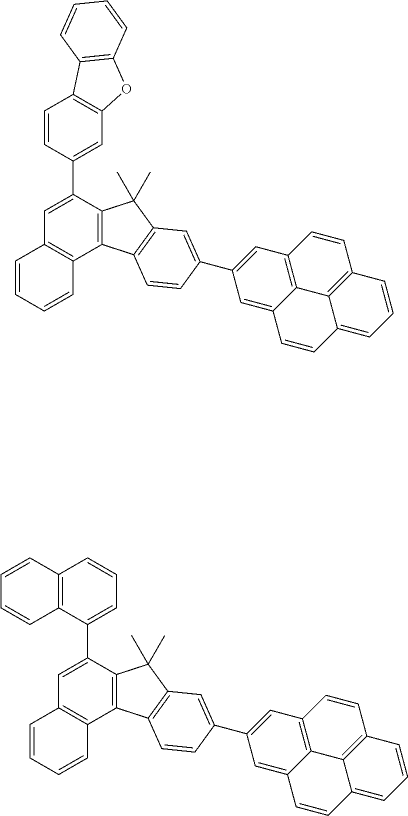

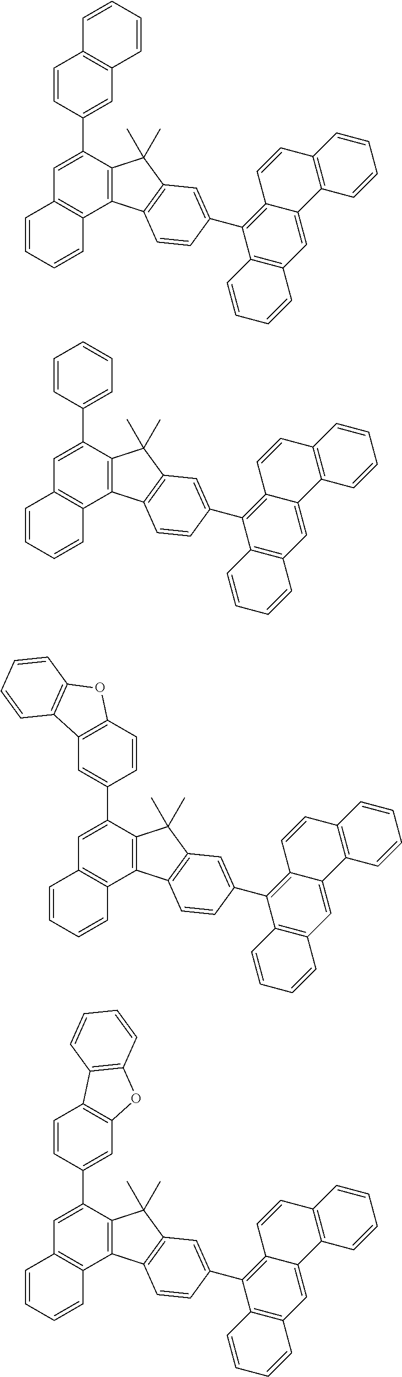

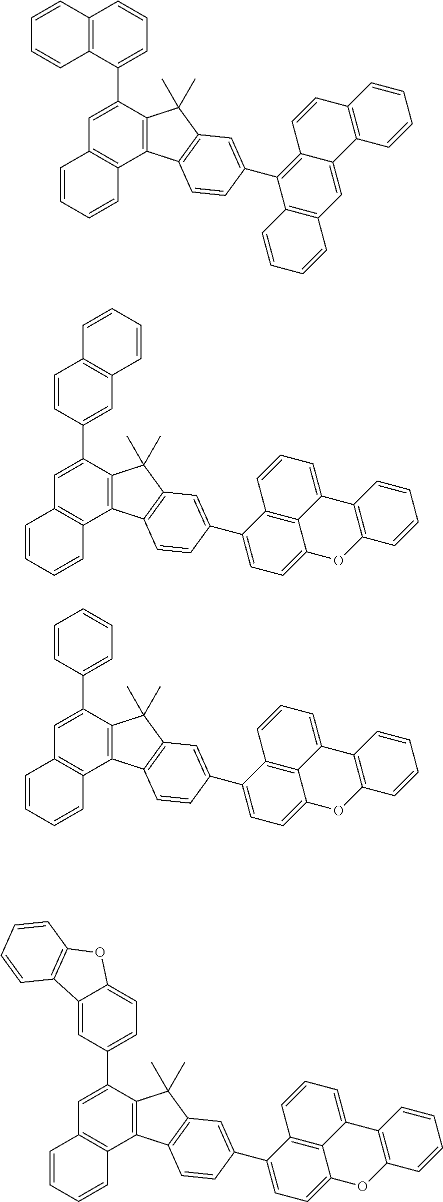

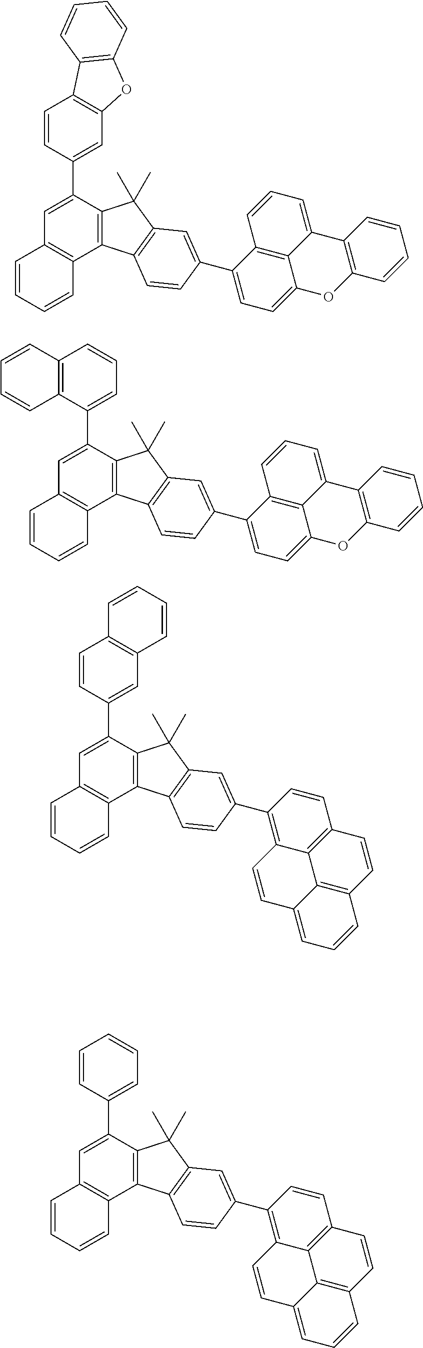

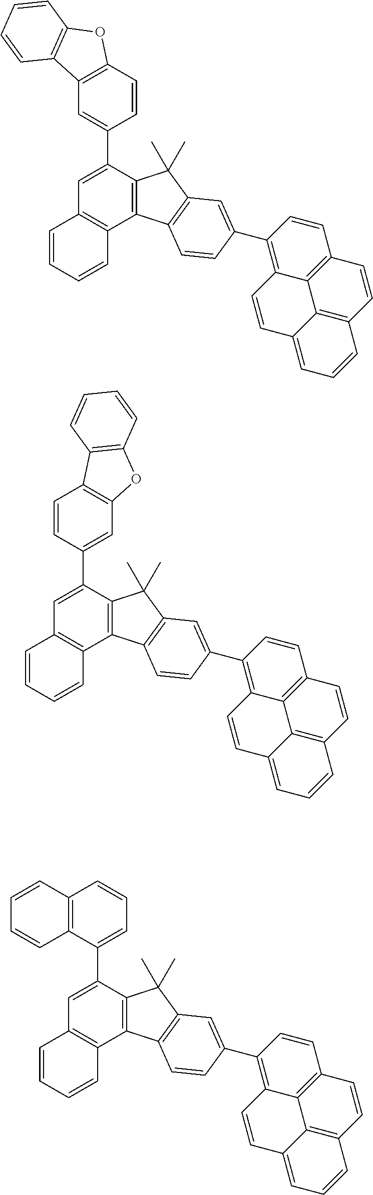

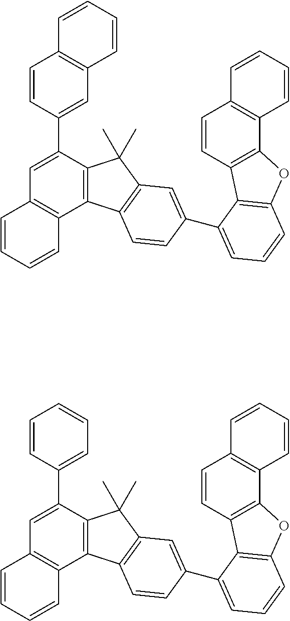 [SMILES: CC1(C)c2cc(-c3cccc4oc5c6ccccc6ccc5c34)ccc2-c2c1c(-c1ccc3ccccc3c1)cc1ccccc21 ; CC1(C)c2cc(-c3cccc4oc5c6ccccc6ccc5c34)ccc2-c2c1c(-c1ccccc1)cc1ccccc21]

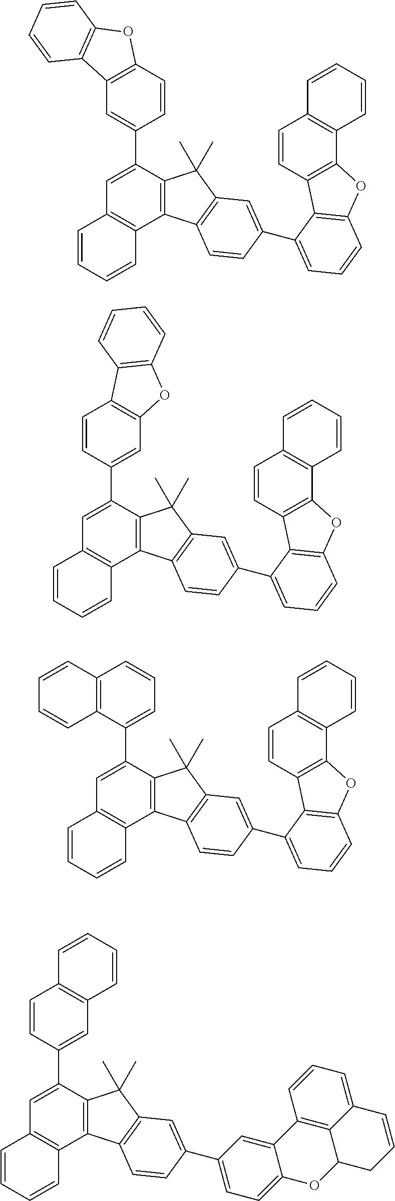

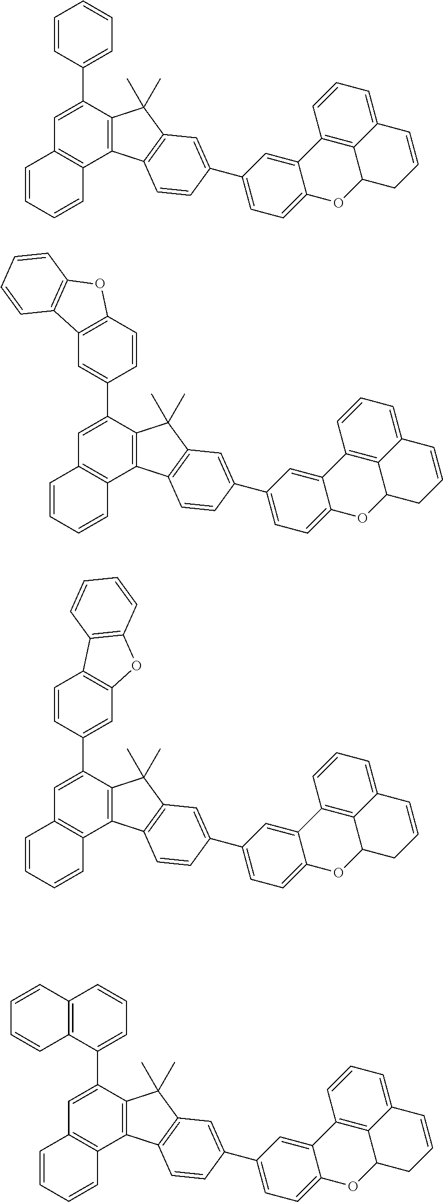

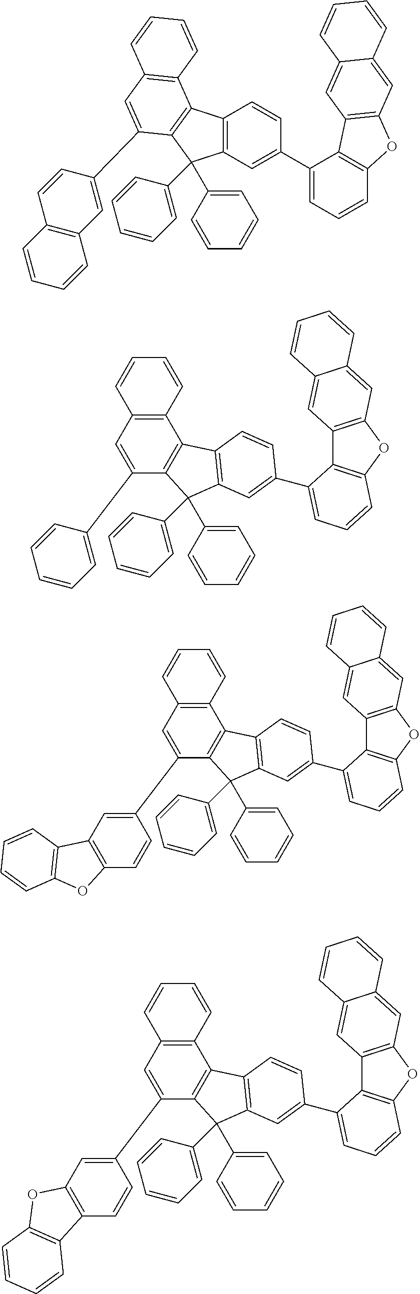 [SMILES: c1ccc(C2(c3ccccc3)c3cc(-c4cccc5oc6cc7ccccc7cc6c45)ccc3-c3c2c(-c2ccc4ccccc4c2)cc2ccccc32)cc1 ; c1ccc(-c2cc3ccccc3c3c2C(c2ccccc2)(c2ccccc2)c2cc(-c4cccc5oc6cc7ccccc7cc6c45)ccc2-3)cc1 ; c1ccc(C2(c3ccccc3)c3cc(-c4cccc5oc6cc7ccccc7cc6c45)ccc3-c3c2c(-c2ccc4oc5ccccc5c4c2)cc2ccccc32)cc1 ; c1ccc(C2(c3ccccc3)c3cc(-c4cccc5oc6cc7ccccc7cc6c45)ccc3-c3c2c(-c2ccc4c(c2)oc2ccccc24)cc2ccccc32)cc1]

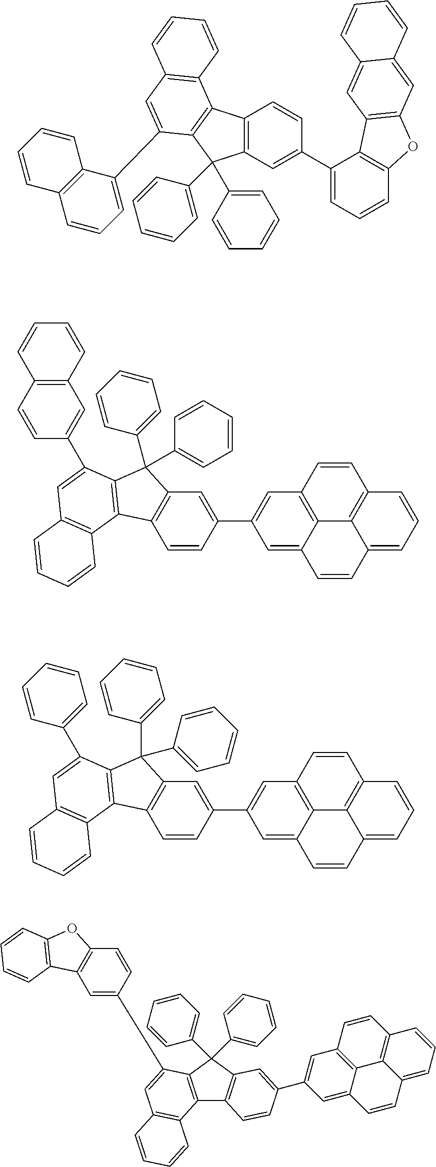

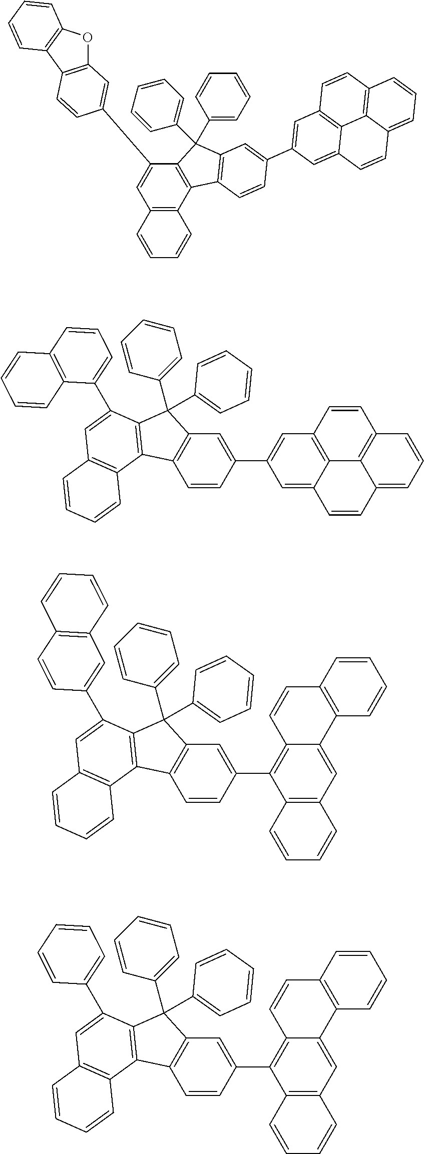

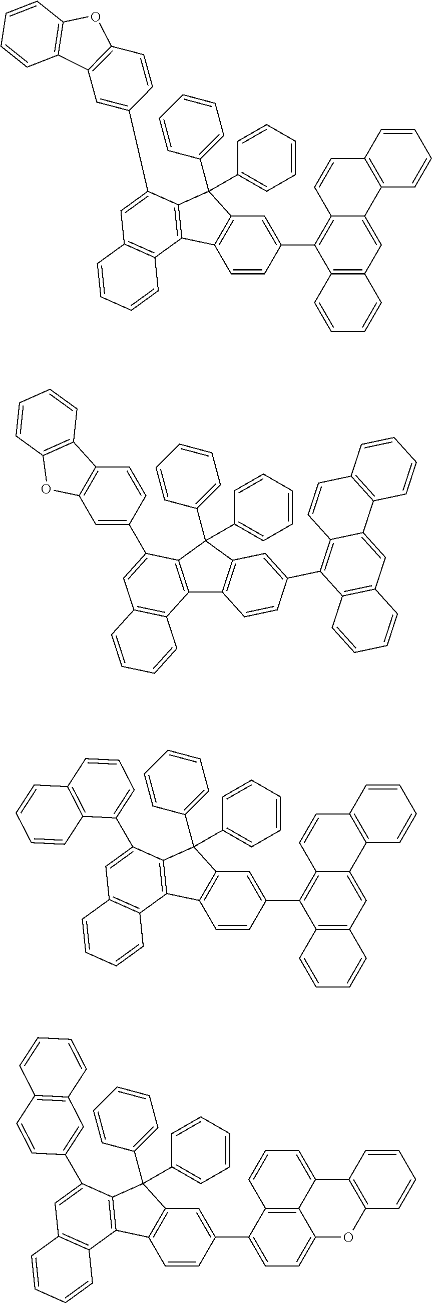

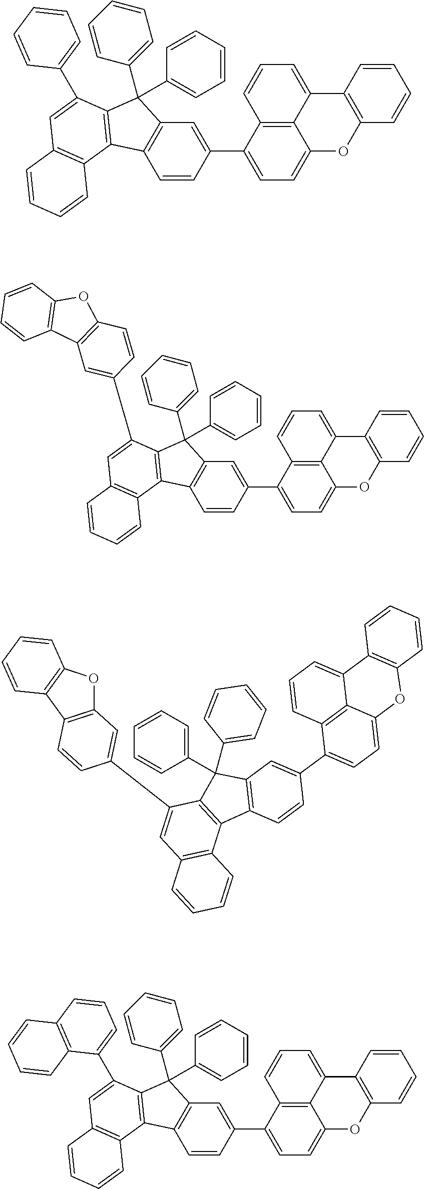 [SMILES: c1ccc(-c2cc3ccccc3c3c2C(c2ccccc2)(c2ccccc2)c2cc(-c4ccc5c6c(cccc46)-c4ccccc4O5)ccc2-3)cc1 ; c1ccc(C2(c3ccccc3)c3cc(-c4ccc5c6c(cccc46)-c4ccccc4O5)ccc3-c3c2c(-c2ccc4oc5ccccc5c4c2)cc2ccccc32)cc1 ; c1ccc(C2(c3ccccc3)c3cc(-c4ccc5c6c(cccc46)-c4ccccc4O5)ccc3-c3c2c(-c2ccc4c(c2)oc2ccccc24)cc2ccccc32)cc1 ; c1ccc(C2(c3ccccc3)c3cc(-c4ccc5c6c(cccc46)-c4ccccc4O5)ccc3-c3c2c(-c2cccc4ccccc24)cc2ccccc32)cc1]

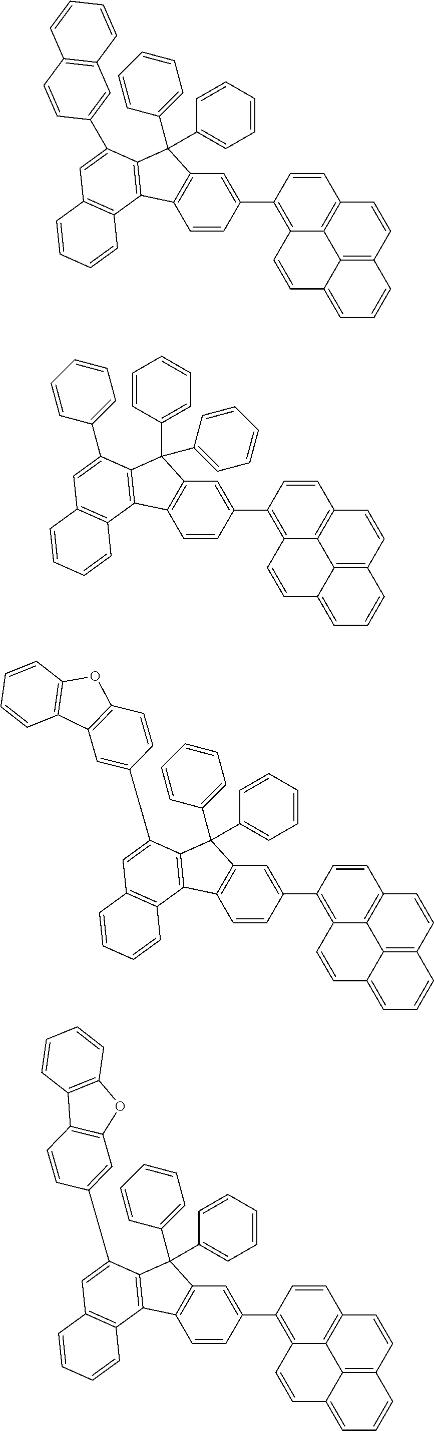

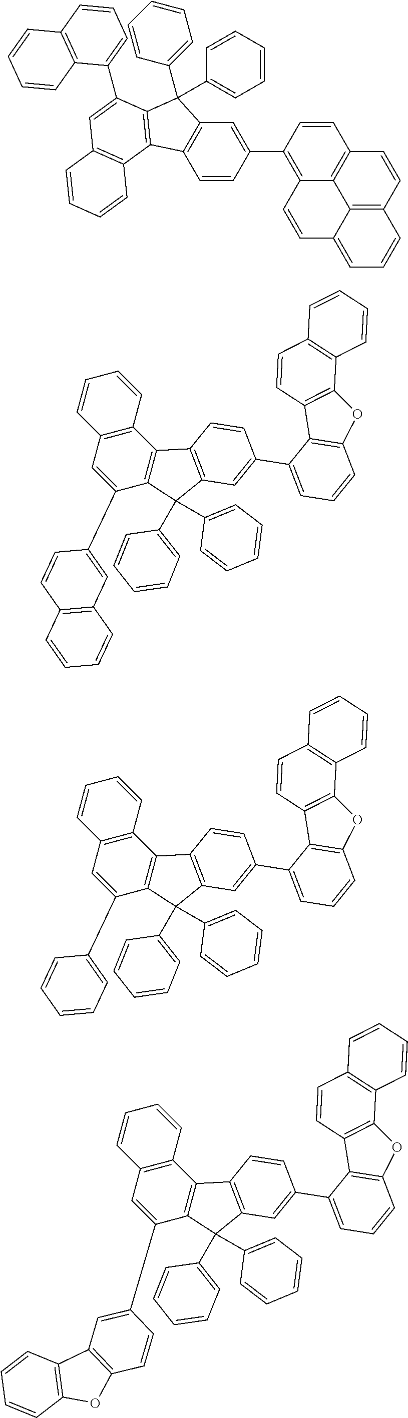

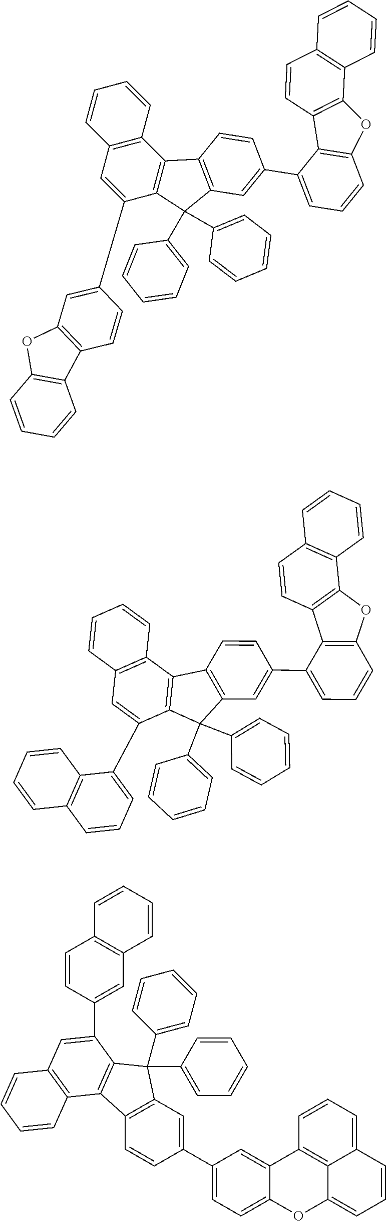 [SMILES: c1ccc(C2(c3ccccc3)c3cc(-c4cccc5oc6c7ccccc7ccc6c45)ccc3-c3c2c(-c2ccc4c(c2)oc2ccccc24)cc2ccccc32)cc1 ; c1ccc(C2(c3ccccc3)c3cc(-c4cccc5oc6c7ccccc7ccc6c45)ccc3-c3c2c(-c2cccc4ccccc24)cc2ccccc32)cc1 ; c1ccc(C2(c3ccccc3)c3cc(-c4ccc5c(c4)-c4cccc6cccc(c46)O5)ccc3-c3c2c(-c2ccc4ccccc4c2)cc2ccccc32)cc1]

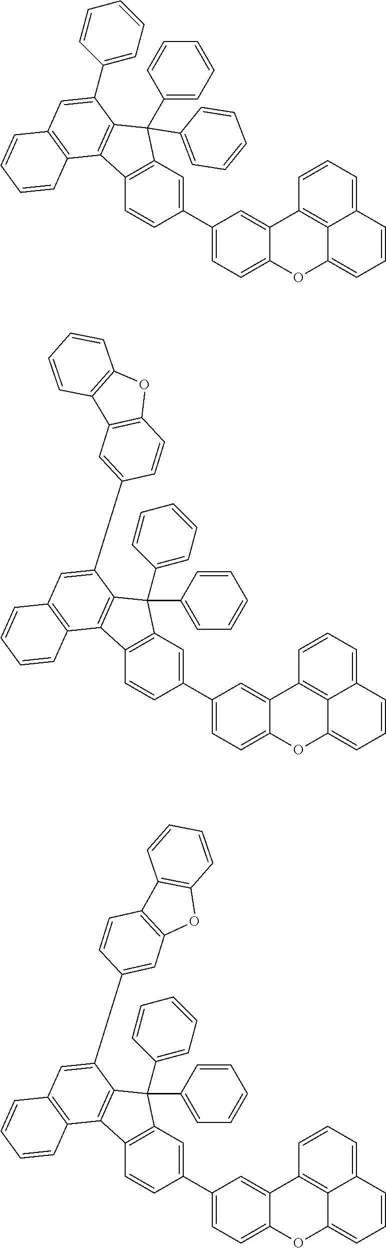 [SMILES: c1ccc(-c2cc3ccccc3c3c2C(c2ccccc2)(c2ccccc2)c2cc(-c4ccc5c(c4)-c4cccc6cccc(c46)O5)ccc2-3)cc1 ; c1ccc(C2(c3ccccc3)c3cc(-c4ccc5c(c4)-c4cccc6cccc(c46)O5)ccc3-c3c2c(-c2ccc4oc5ccccc5c4c2)cc2ccccc32)cc1 ; c1ccc(C2(c3ccccc3)c3cc(-c4ccc5c(c4)-c4cccc6cccc(c46)O5)ccc3-c3c2c(-c2ccc4c(c2)oc2ccccc24)cc2ccccc32)cc1]

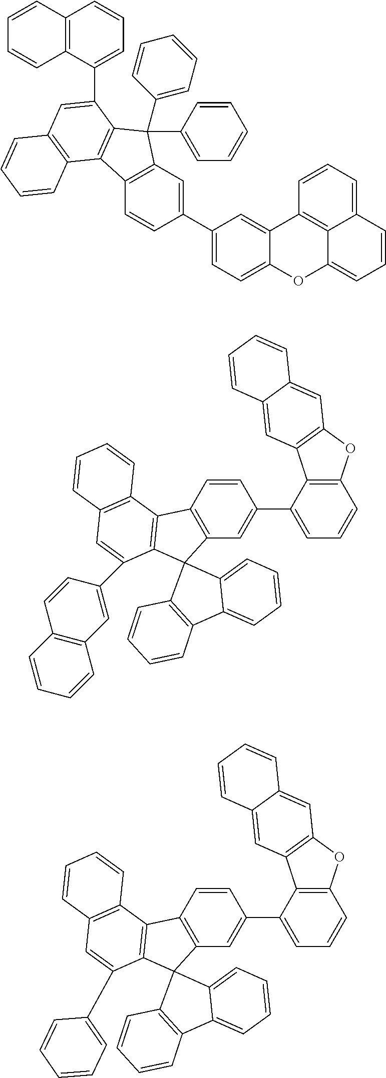 [SMILES: c1ccc(C2(c3ccccc3)c3cc(-c4ccc5c(c4)-c4cccc6cccc(c46)O5)ccc3-c3c2c(-c2cccc4ccccc24)cc2ccccc32)cc1 ; c1ccc2c(c1)-c1ccccc1C21c2cc(-c3cccc4oc5cc6ccccc6cc5c34)ccc2-c2c1c(-c1ccc3ccccc3c1)cc1ccccc21 ; c1ccc(-c2cc3ccccc3c3c2C2(c4ccccc4-c4ccccc42)c2cc(-c4cccc5oc6cc7ccccc7cc6c45)ccc2-3)cc1]

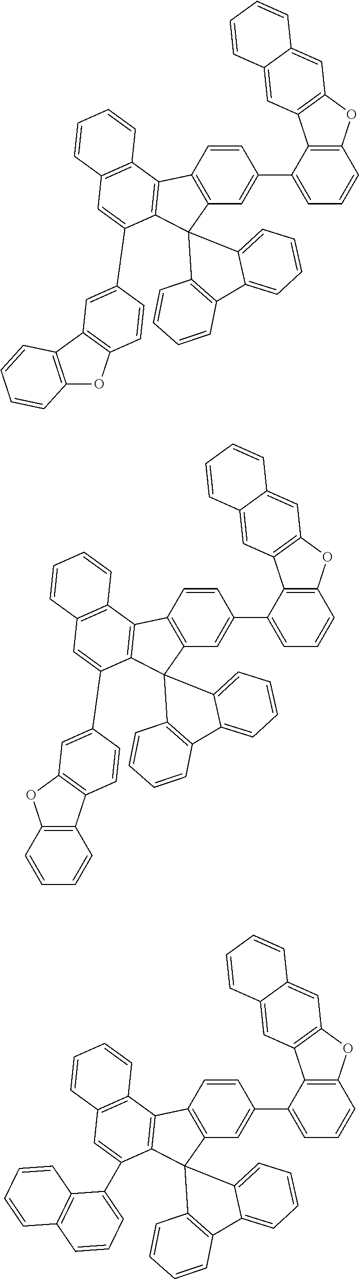 [SMILES: c1ccc2c(c1)-c1ccccc1C21c2cc(-c3cccc4oc5cc6ccccc6cc5c34)ccc2-c2c1c(-c1ccc3oc4ccccc4c3c1)cc1ccccc21 ; c1ccc2c(c1)-c1ccccc1C21c2cc(-c3cccc4oc5cc6ccccc6cc5c34)ccc2-c2c1c(-c1ccc3c(c1)oc1ccccc13)cc1ccccc21 ; c1ccc2c(c1)-c1ccccc1C21c2cc(-c3cccc4oc5cc6ccccc6cc5c34)ccc2-c2c1c(-c1cccc3ccccc13)cc1ccccc21]

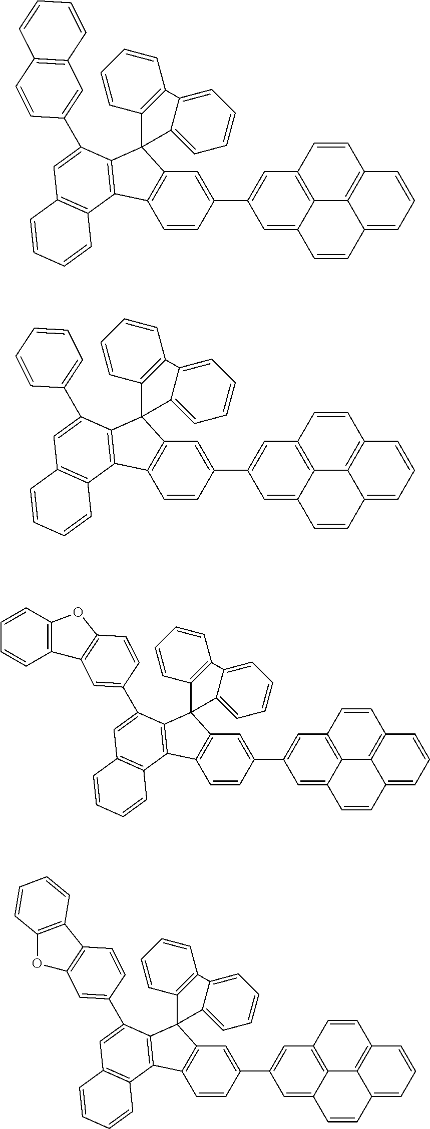

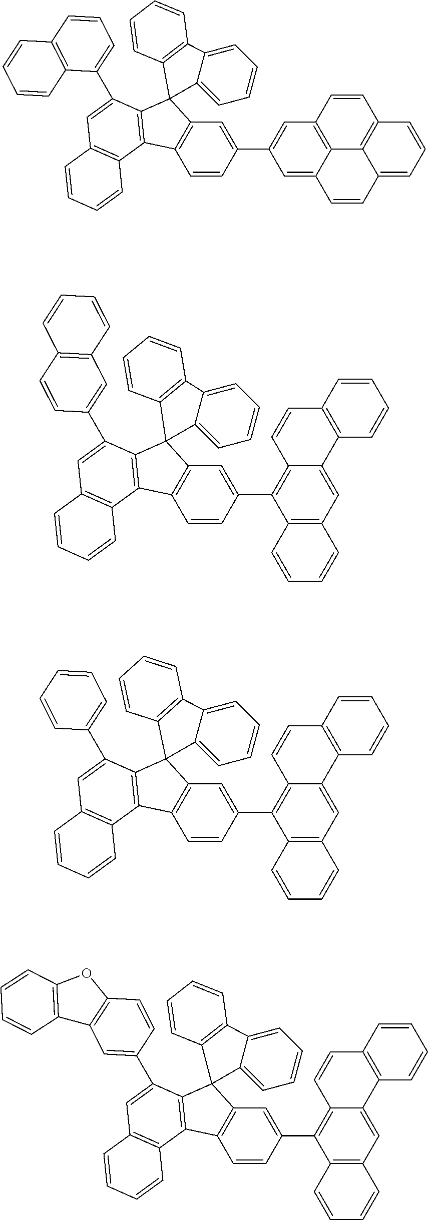

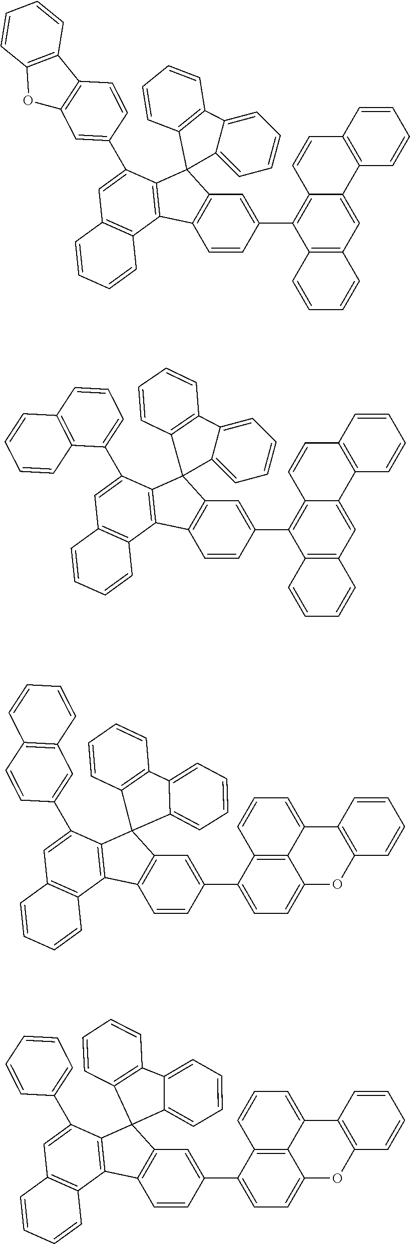

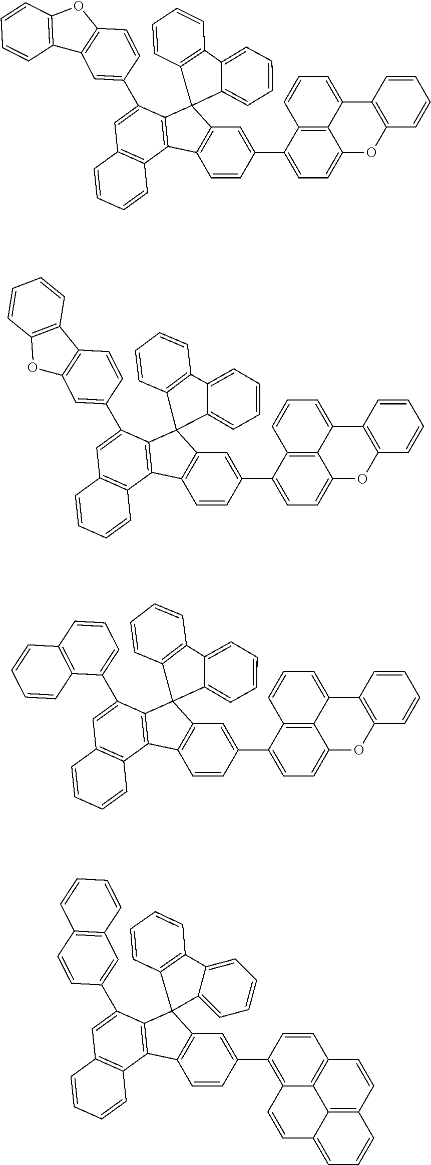

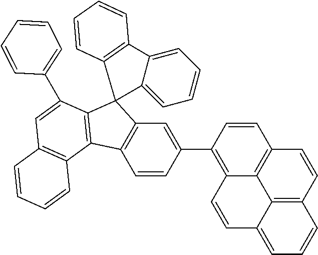 [SMILES: c1ccc(-c2cc3ccccc3c3c2C2(c4ccccc4-c4ccccc42)c2cc(-c4ccc5ccc6cccc7ccc4c5c67)ccc2-3)cc1]

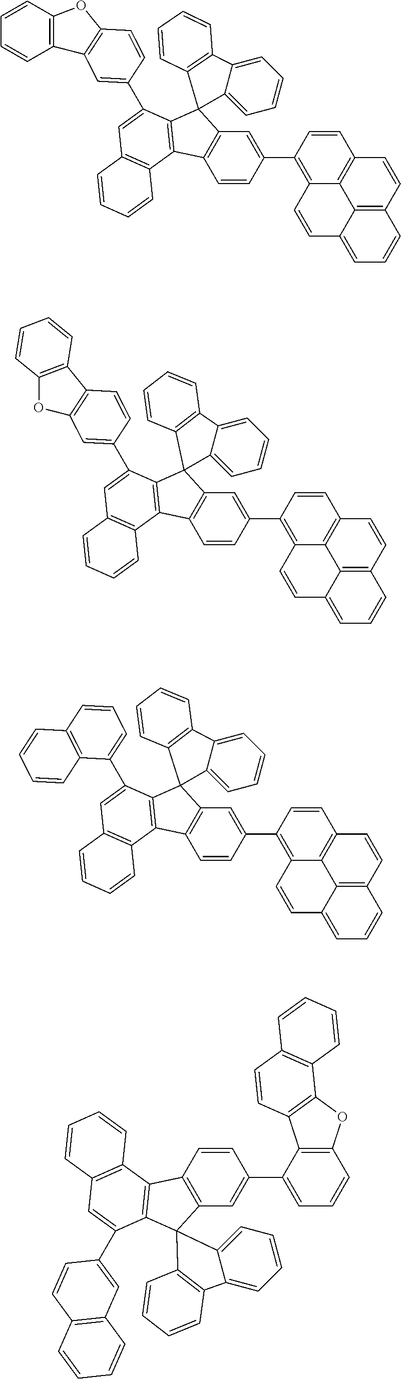

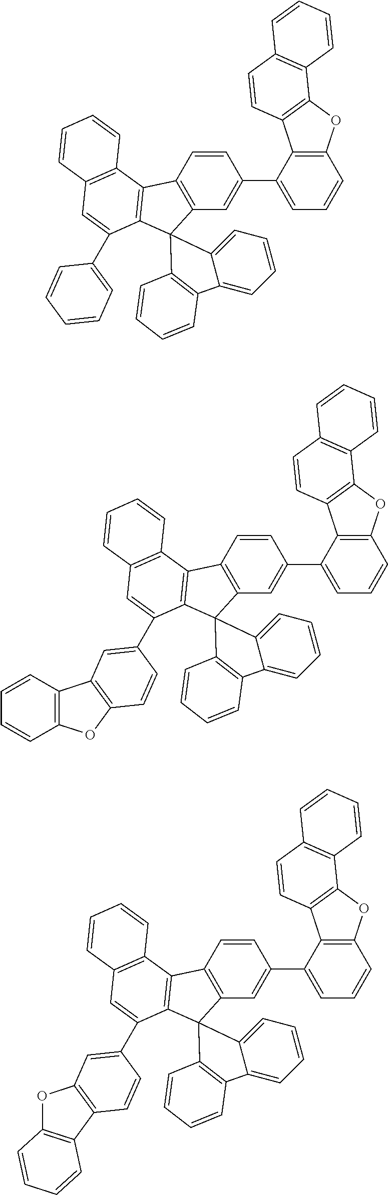 [SMILES: c1ccc(-c2cc3ccccc3c3c2C2(c4ccccc4-c4ccccc42)c2cc(-c4cccc5oc6c7ccccc7ccc6c45)ccc2-3)cc1 ; c1ccc2c(c1)-c1ccccc1C21c2cc(-c3cccc4oc5c6ccccc6ccc5c34)ccc2-c2c1c(-c1ccc3oc4ccccc4c3c1)cc1ccccc21 ; c1ccc2c(c1)-c1ccccc1C21c2cc(-c3cccc4oc5c6ccccc6ccc5c34)ccc2-c2c1c(-c1ccc3c(c1)oc1ccccc13)cc1ccccc21]

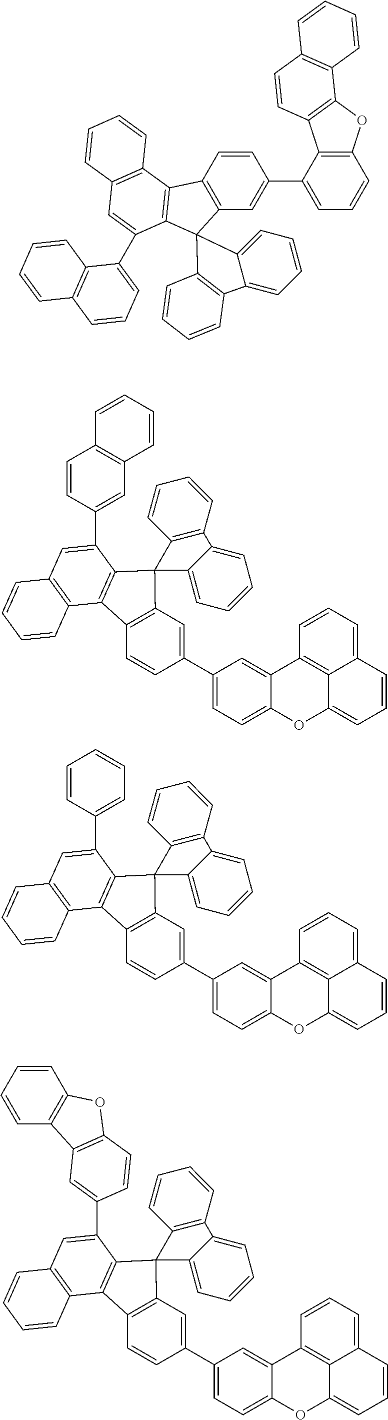 [SMILES: c1ccc2c(c1)-c1ccccc1C21c2cc(-c3cccc4oc5c6ccccc6ccc5c34)ccc2-c2c1c(-c1cccc3ccccc13)cc1ccccc21 ; c1ccc2c(c1)-c1ccccc1C21c2cc(-c3ccc4c(c3)-c3cccc5cccc(c35)O4)ccc2-c2c1c(-c1ccc3ccccc3c1)cc1ccccc21 ; c1ccc(-c2cc3ccccc3c3c2C2(c4ccccc4-c4ccccc42)c2cc(-c4ccc5c(c4)-c4cccc6cccc(c46)O5)ccc2-3)cc1 ; c1ccc2c(c1)-c1ccccc1C21c2cc(-c3ccc4c(c3)-c3cccc5cccc(c35)O4)ccc2-c2c1c(-c1ccc3oc4ccccc4c3c1)cc1ccccc21]

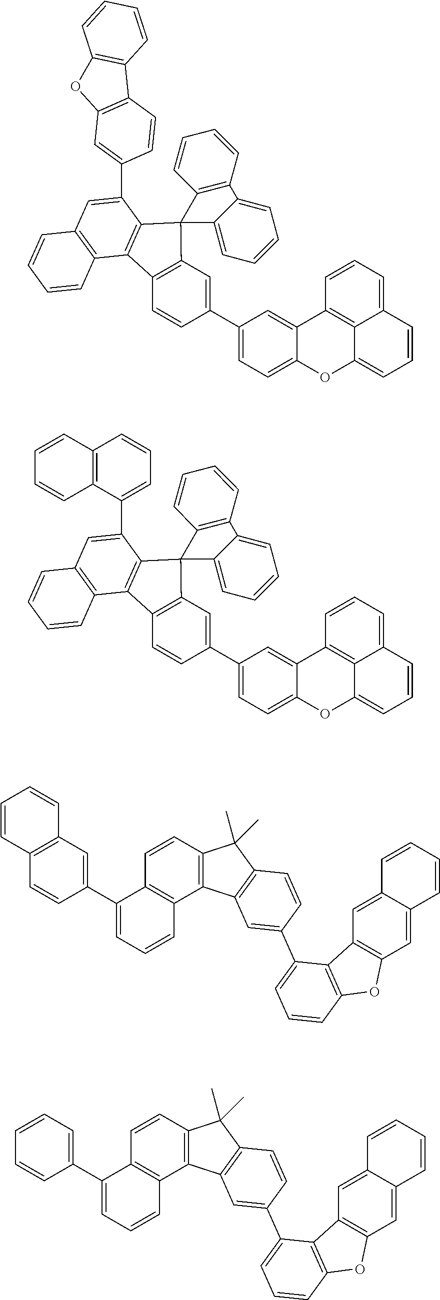 [SMILES: c1ccc2c(c1)-c1ccccc1C21c2cc(-c3ccc4c(c3)-c3cccc5cccc(c35)O4)ccc2-c2c1c(-c1ccc3c(c1)oc1ccccc13)cc1ccccc21 ; c1ccc2c(c1)-c1ccccc1C21c2cc(-c3ccc4c(c3)-c3cccc5cccc(c35)O4)ccc2-c2c1c(-c1cccc3ccccc13)cc1ccccc21 ; CC1(C)c2ccc(-c3cccc4oc5cc6ccccc6cc5c34)cc2-c2c1ccc1c(-c3ccc4ccccc4c3)cccc21 ; CC1(C)c2ccc(-c3cccc4oc5cc6ccccc6cc5c34)cc2-c2c1ccc1c(-c3ccccc3)cccc21]

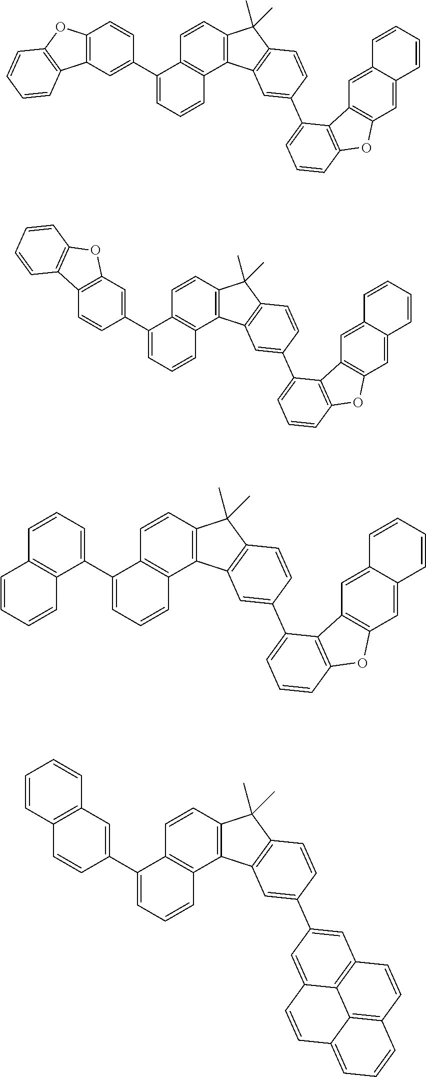

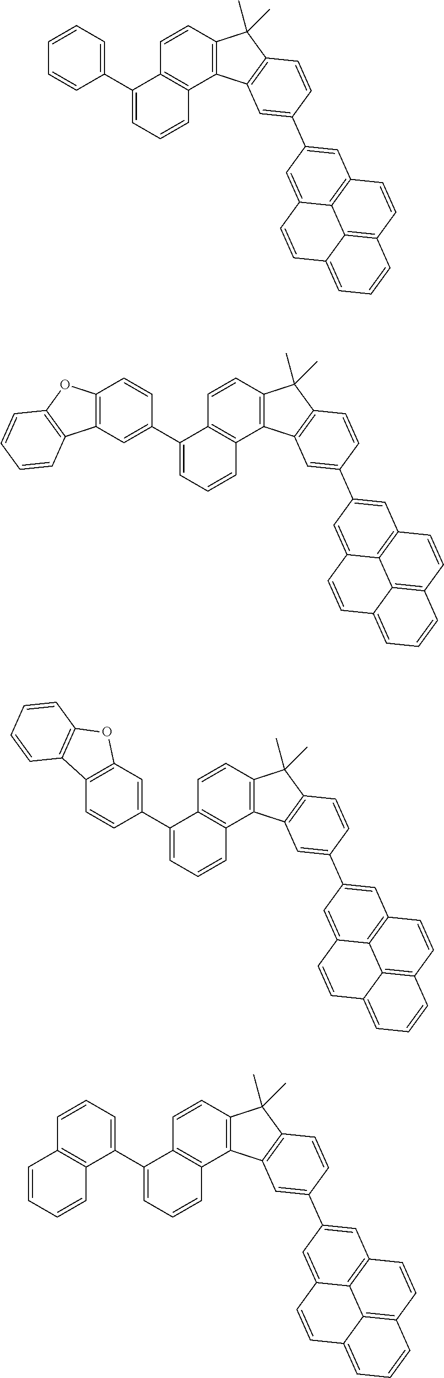

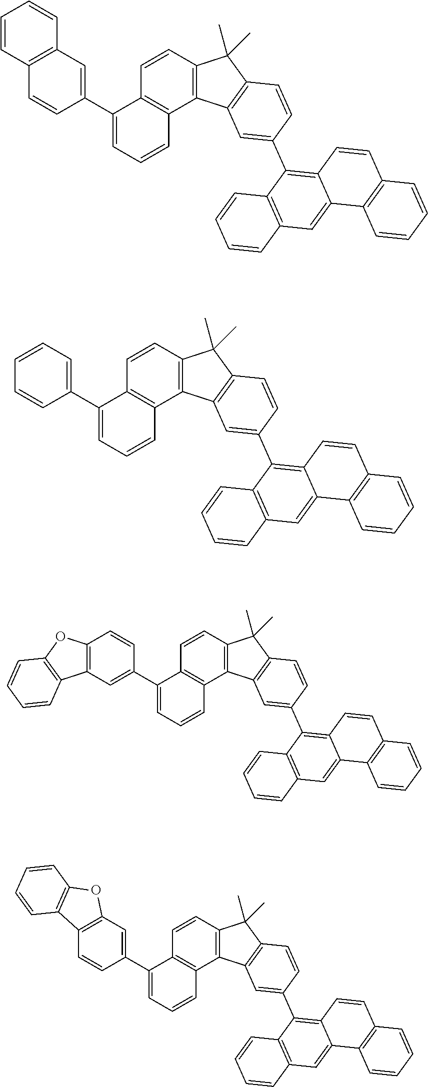

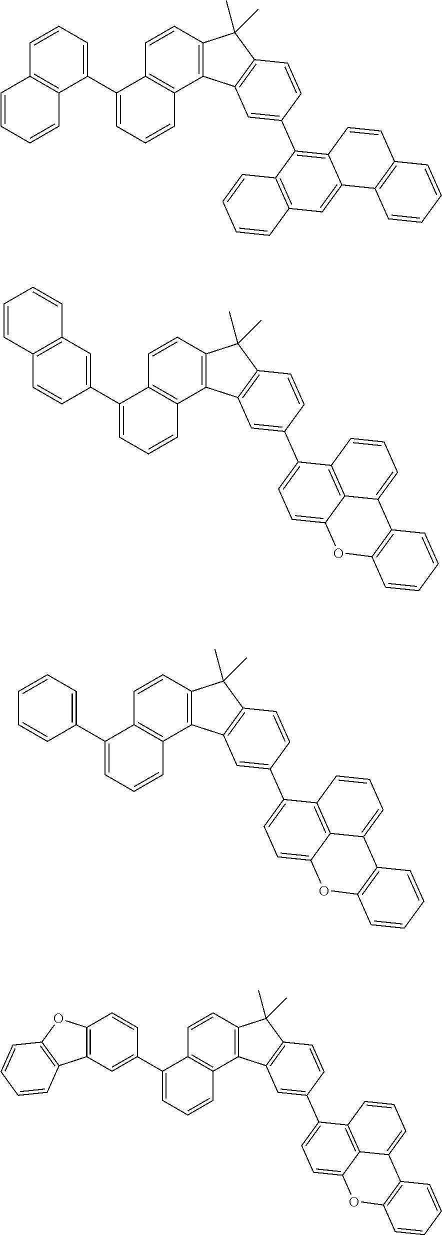

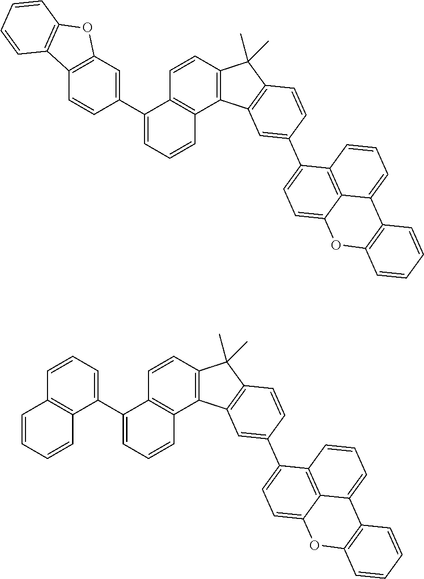 [SMILES: CC1(C)c2ccc(-c3ccc4c5c(cccc35)-c3ccccc3O4)cc2-c2c1ccc1c(-c3ccc4c(c3)oc3ccccc34)cccc21 ; CC1(C)c2ccc(-c3ccc4c5c(cccc35)-c3ccccc3O4)cc2-c2c1ccc1c(-c3cccc4ccccc34)cccc21]

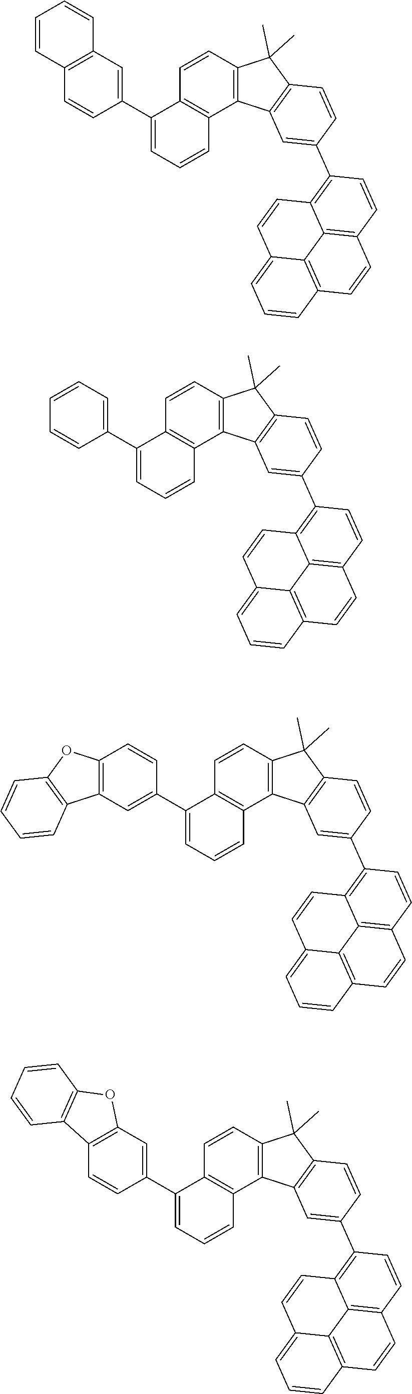

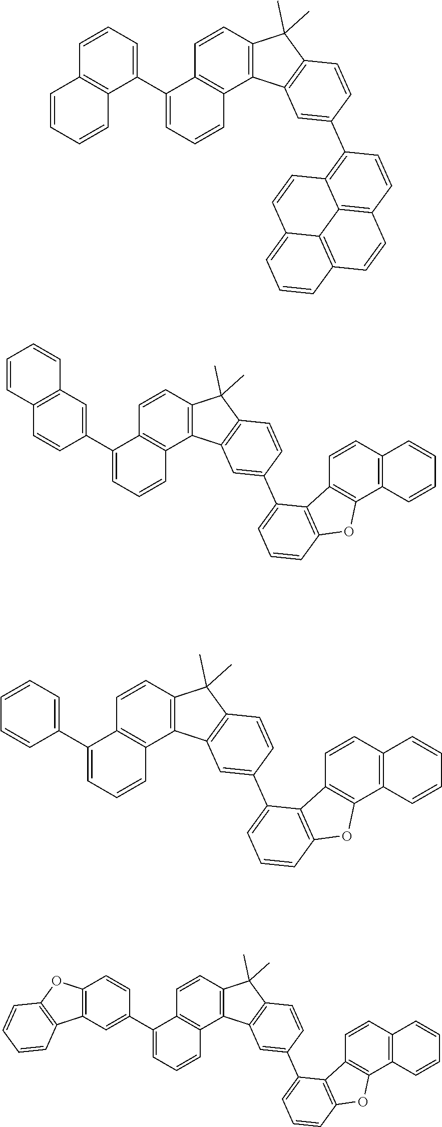

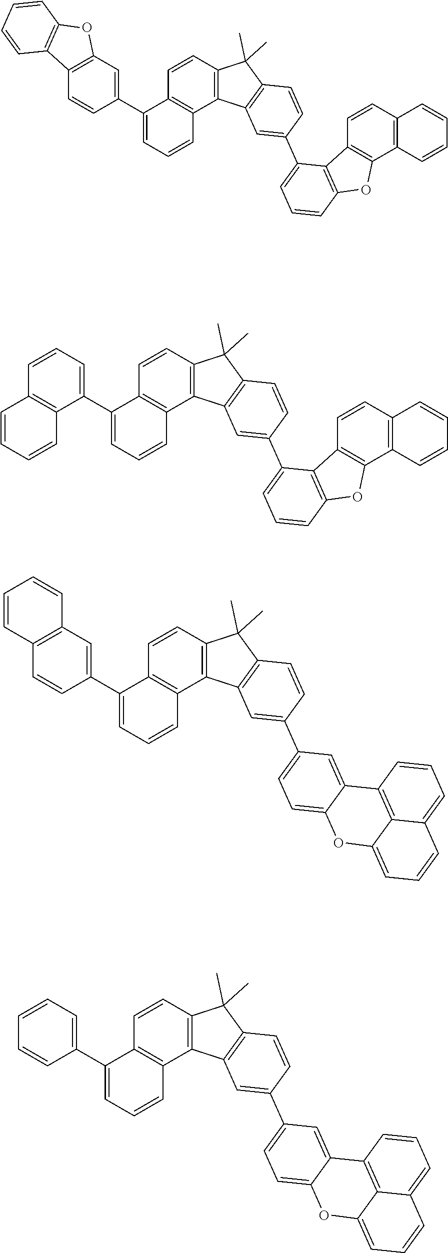 [SMILES: CC1(C)c2ccc(-c3cccc4oc5c6ccccc6ccc5c34)cc2-c2c1ccc1c(-c3ccc4c(c3)oc3ccccc34)cccc21 ; CC1(C)c2ccc(-c3cccc4oc5c6ccccc6ccc5c34)cc2-c2c1ccc1c(-c3cccc4ccccc34)cccc21 ; CC1(C)c2ccc(-c3ccc4c(c3)-c3cccc5cccc(c35)O4)cc2-c2c1ccc1c(-c3ccc4ccccc4c3)cccc21 ; CC1(C)c2ccc(-c3ccc4c(c3)-c3cccc5cccc(c35)O4)cc2-c2c1ccc1c(-c3ccccc3)cccc21]

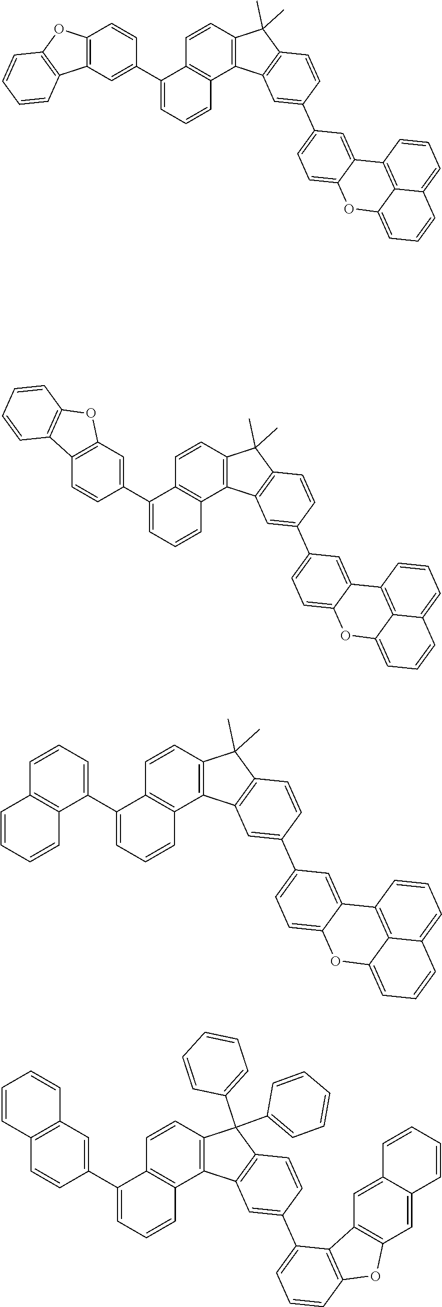 [SMILES: CC1(C)c2ccc(-c3ccc4c(c3)-c3cccc5cccc(c35)O4)cc2-c2c1ccc1c(-c3ccc4oc5ccccc5c4c3)cccc21 ; CC1(C)c2ccc(-c3ccc4c(c3)-c3cccc5cccc(c35)O4)cc2-c2c1ccc1c(-c3ccc4c(c3)oc3ccccc34)cccc21 ; CC1(C)c2ccc(-c3ccc4c(c3)-c3cccc5cccc(c35)O4)cc2-c2c1ccc1c(-c3cccc4ccccc34)cccc21 ; c1ccc(C2(c3ccccc3)c3ccc(-c4cccc5oc6cc7ccccc7cc6c45)cc3-c3c2ccc2c(-c4ccc5ccccc5c4)cccc32)cc1]

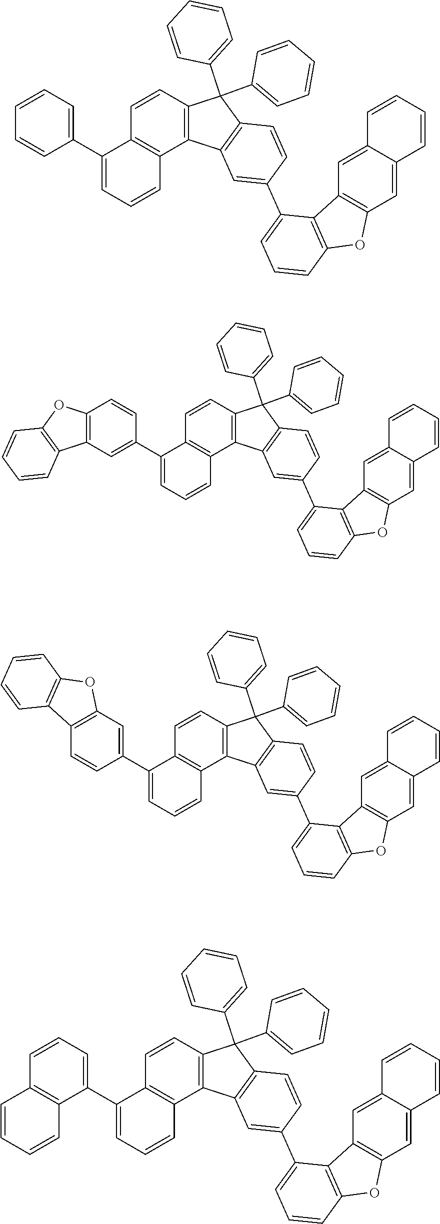 [SMILES: c1ccc(-c2cccc3c4c(ccc23)C(c2ccccc2)(c2ccccc2)c2ccc(-c3cccc5oc6cc7ccccc7cc6c35)cc2-4)cc1 ; c1ccc(C2(c3ccccc3)c3ccc(-c4cccc5oc6cc7ccccc7cc6c45)cc3-c3c2ccc2c(-c4ccc5oc6ccccc6c5c4)cccc32)cc1 ; c1ccc(C2(c3ccccc3)c3ccc(-c4cccc5oc6cc7ccccc7cc6c45)cc3-c3c2ccc2c(-c4ccc5c(c4)oc4ccccc45)cccc32)cc1 ; c1ccc(C2(c3ccccc3)c3ccc(-c4cccc5oc6cc7ccccc7cc6c45)cc3-c3c2ccc2c(-c4cccc5ccccc45)cccc32)cc1]

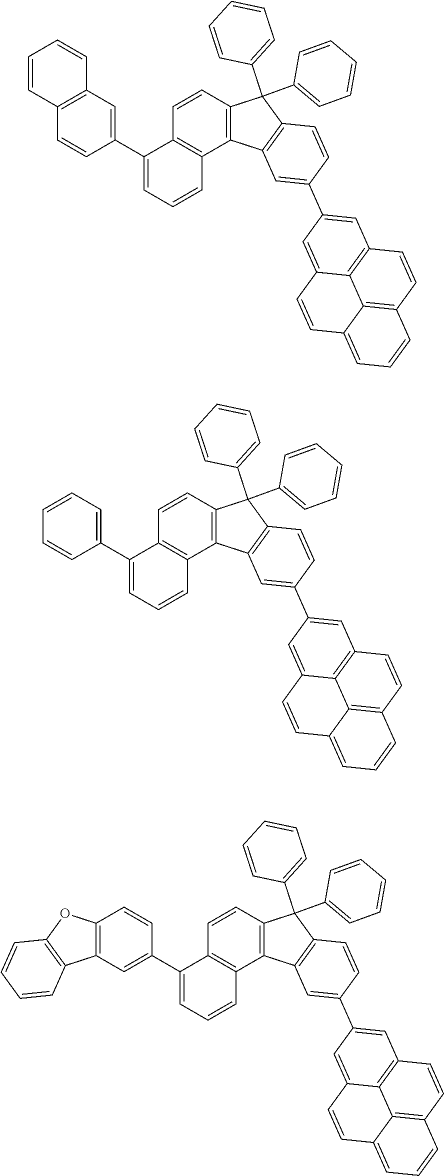

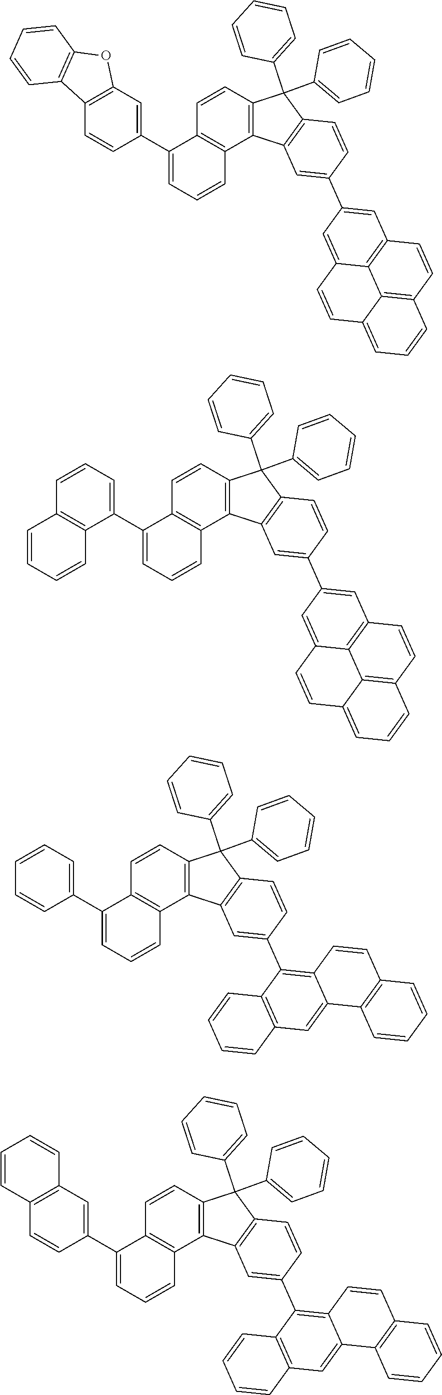

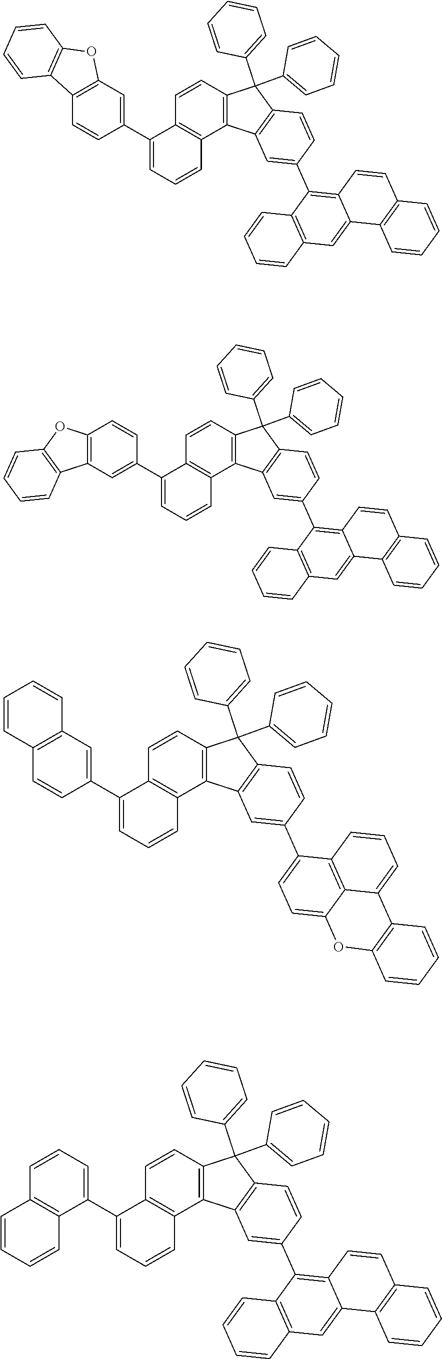

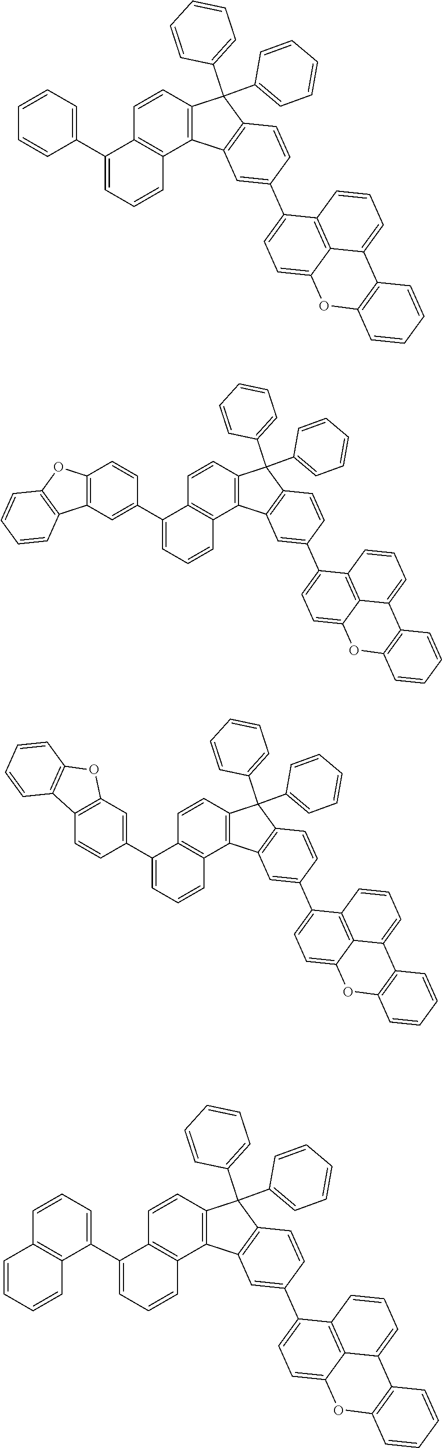 [SMILES: c1ccc(-c2cccc3c4c(ccc23)C(c2ccccc2)(c2ccccc2)c2ccc(-c3ccc5c6c(cccc36)-c3ccccc3O5)cc2-4)cc1 ; c1ccc(C2(c3ccccc3)c3ccc(-c4ccc5c6c(cccc46)-c4ccccc4O5)cc3-c3c2ccc2c(-c4ccc5oc6ccccc6c5c4)cccc32)cc1 ; c1ccc(C2(c3ccccc3)c3ccc(-c4ccc5c6c(cccc46)-c4ccccc4O5)cc3-c3c2ccc2c(-c4ccc5c(c4)oc4ccccc45)cccc32)cc1 ; c1ccc(C2(c3ccccc3)c3ccc(-c4ccc5c6c(cccc46)-c4ccccc4O5)cc3-c3c2ccc2c(-c4cccc5ccccc45)cccc32)cc1]

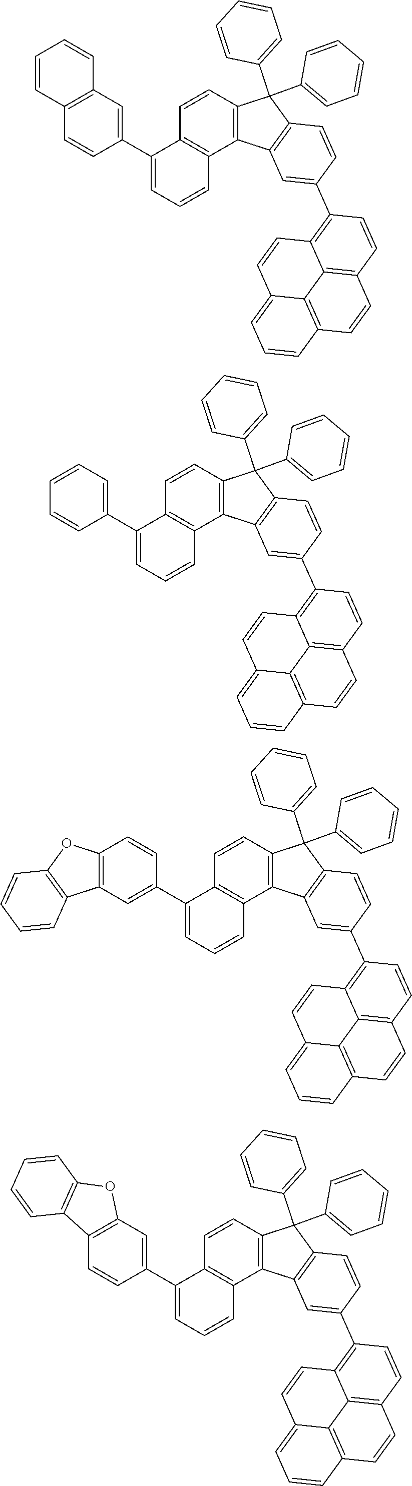

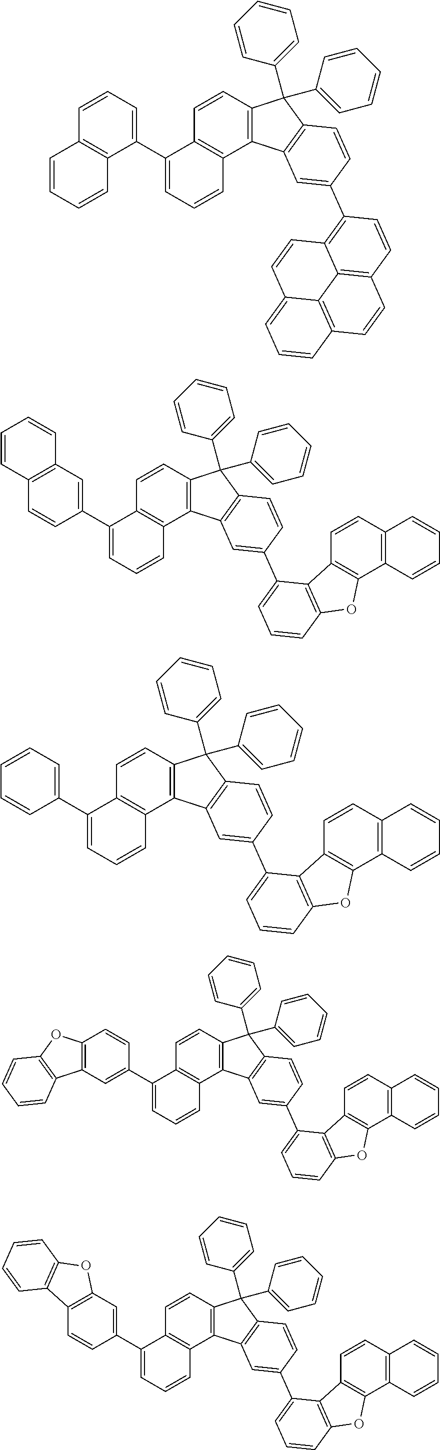

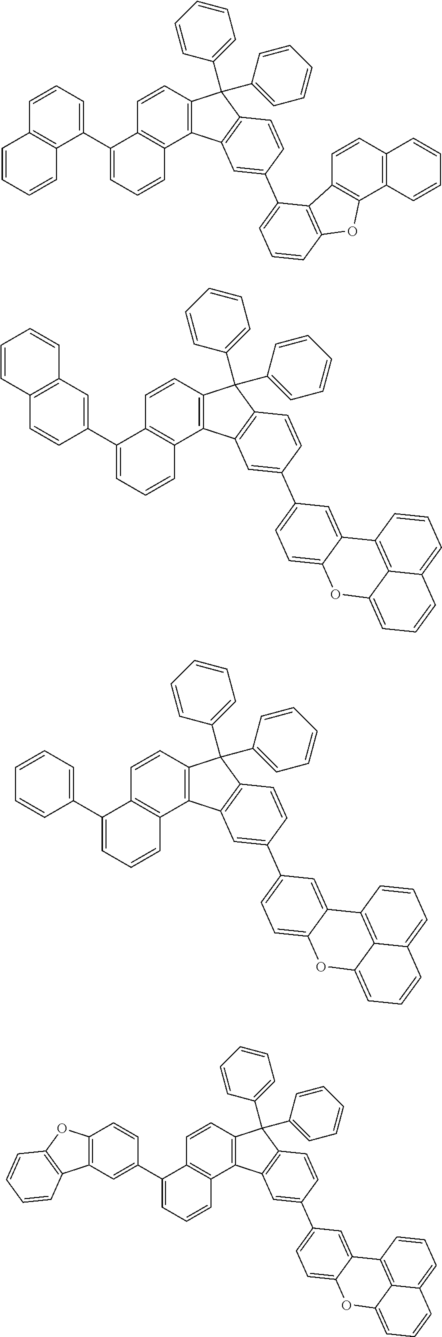 [SMILES: c1ccc(C2(c3ccccc3)c3ccc(-c4cccc5oc6c7ccccc7ccc6c45)cc3-c3c2ccc2c(-c4cccc5ccccc45)cccc32)cc1 ; c1ccc(C2(c3ccccc3)c3ccc(-c4ccc5c(c4)-c4cccc6cccc(c46)O5)cc3-c3c2ccc2c(-c4ccc5ccccc5c4)cccc32)cc1 ; c1ccc(-c2cccc3c4c(ccc23)C(c2ccccc2)(c2ccccc2)c2ccc(-c3ccc5c(c3)-c3cccc6cccc(c36)O5)cc2-4)cc1 ; c1ccc(C2(c3ccccc3)c3ccc(-c4ccc5c(c4)-c4cccc6cccc(c46)O5)cc3-c3c2ccc2c(-c4ccc5oc6ccccc6c5c4)cccc32)cc1]

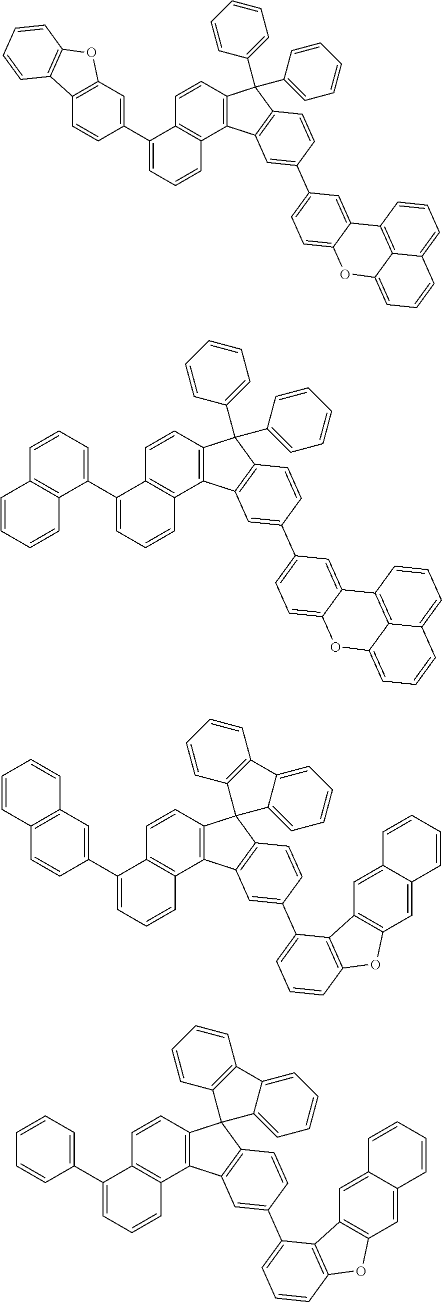 [SMILES: c1ccc(C2(c3ccccc3)c3ccc(-c4ccc5c(c4)-c4cccc6cccc(c46)O5)cc3-c3c2ccc2c(-c4ccc5c(c4)oc4ccccc45)cccc32)cc1 ; c1ccc(C2(c3ccccc3)c3ccc(-c4ccc5c(c4)-c4cccc6cccc(c46)O5)cc3-c3c2ccc2c(-c4cccc5ccccc45)cccc32)cc1 ; c1ccc2c(c1)-c1ccccc1C21c2ccc(-c3cccc4oc5cc6ccccc6cc5c34)cc2-c2c1ccc1c(-c3ccc4ccccc4c3)cccc21 ; c1ccc(-c2cccc3c4c(ccc23)C2(c3ccccc3-c3ccccc32)c2ccc(-c3cccc5oc6cc7ccccc7cc6c35)cc2-4)cc1]

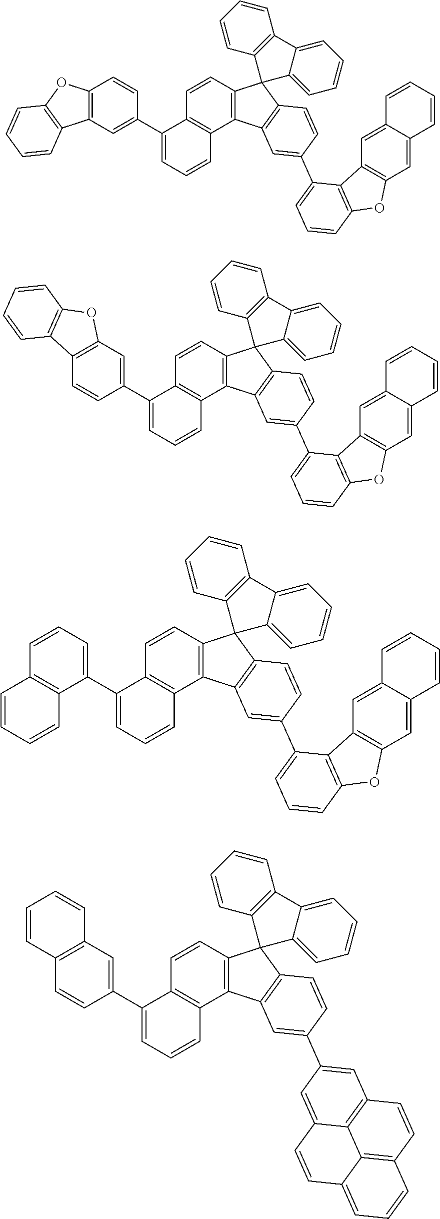

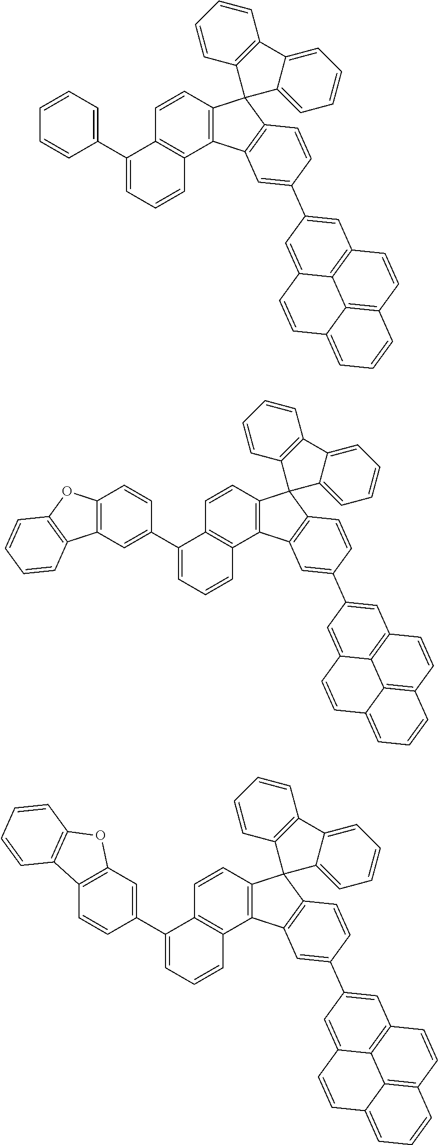

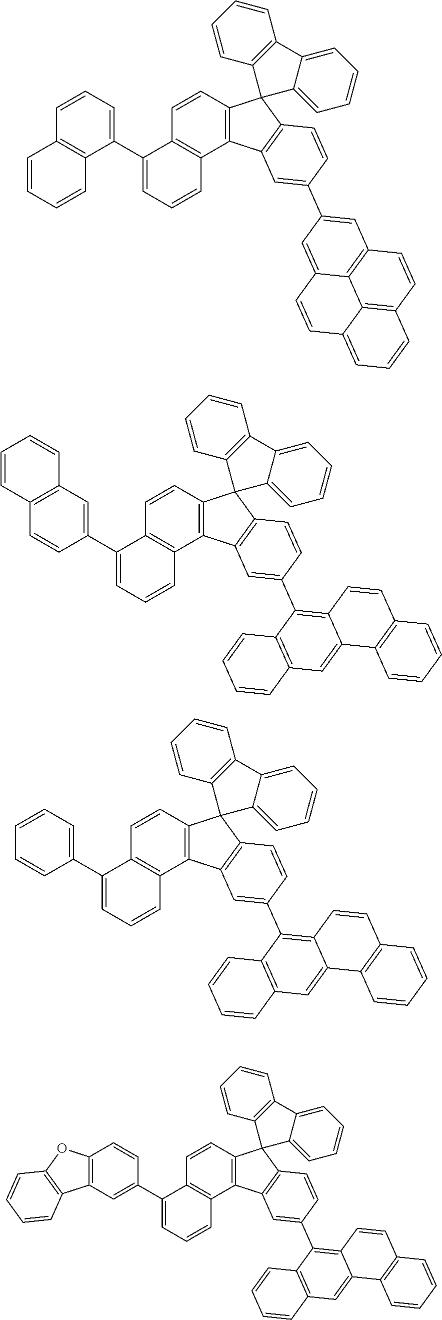

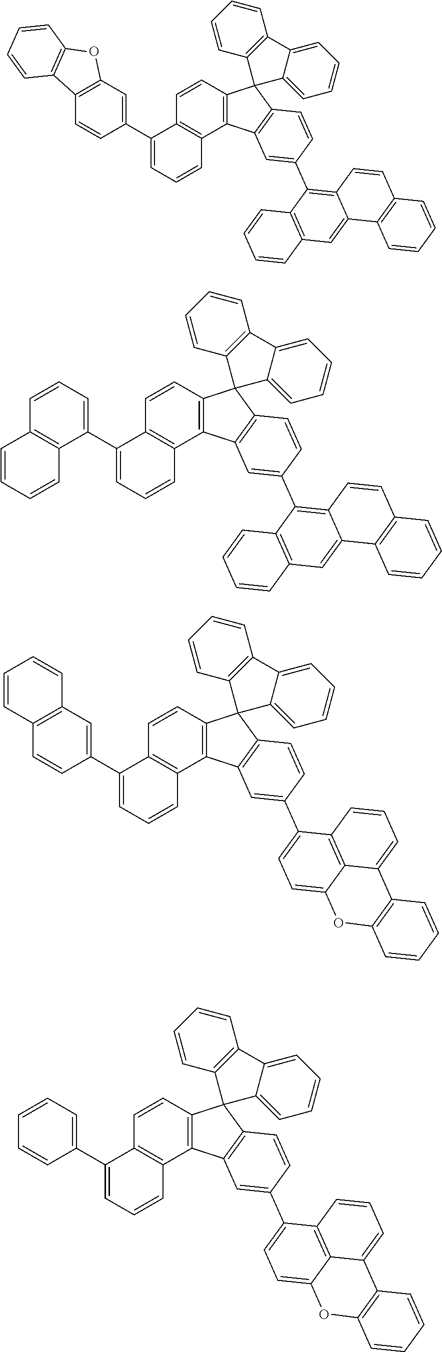

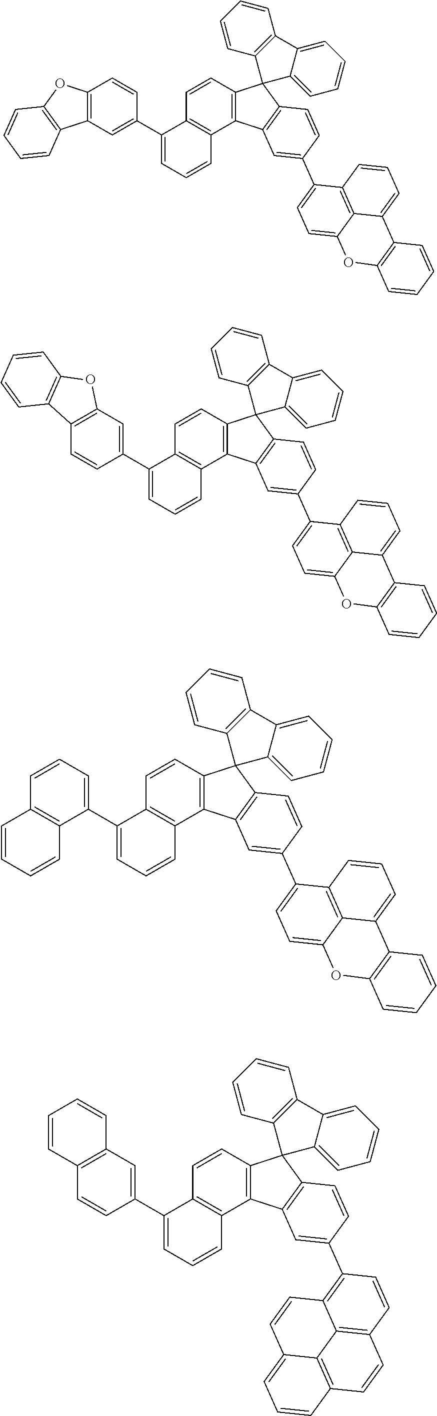

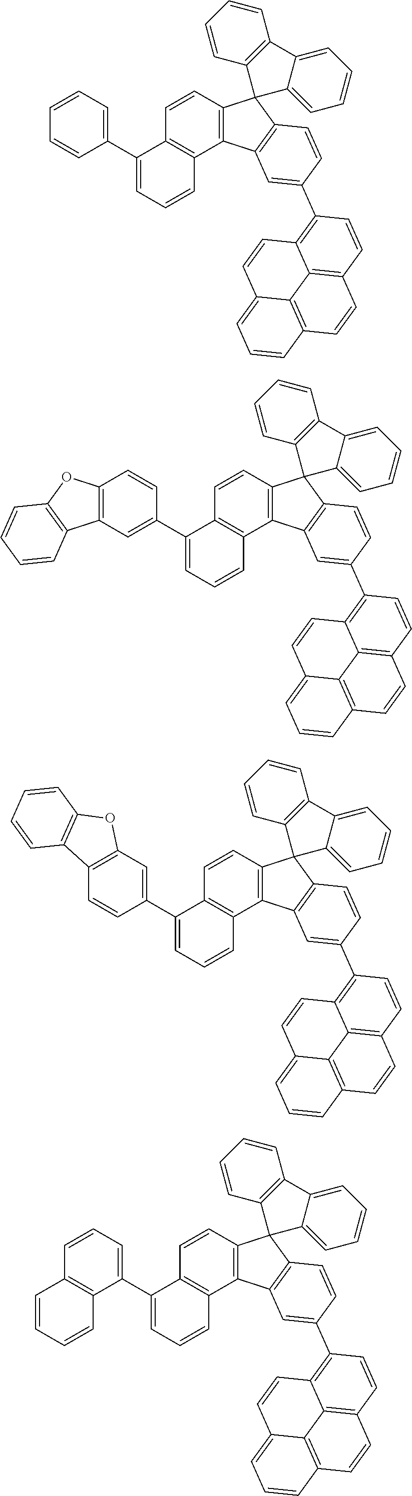

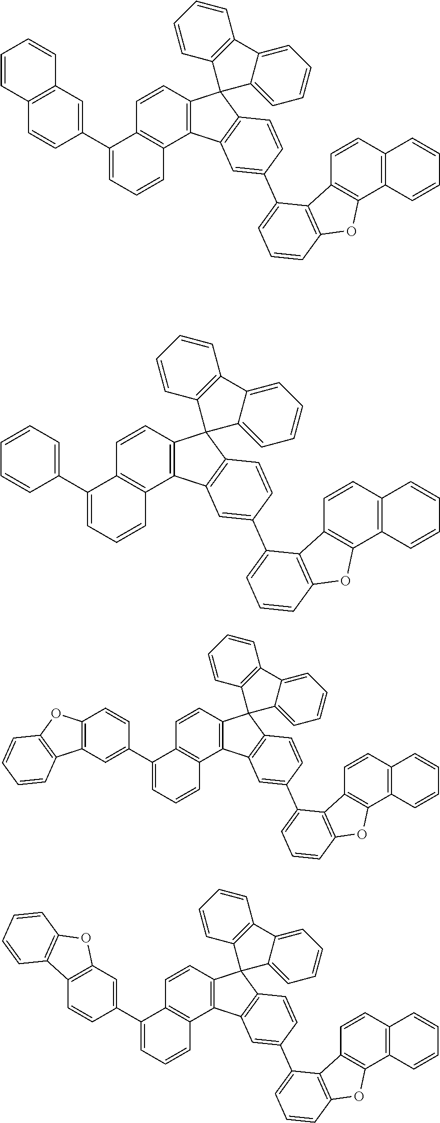

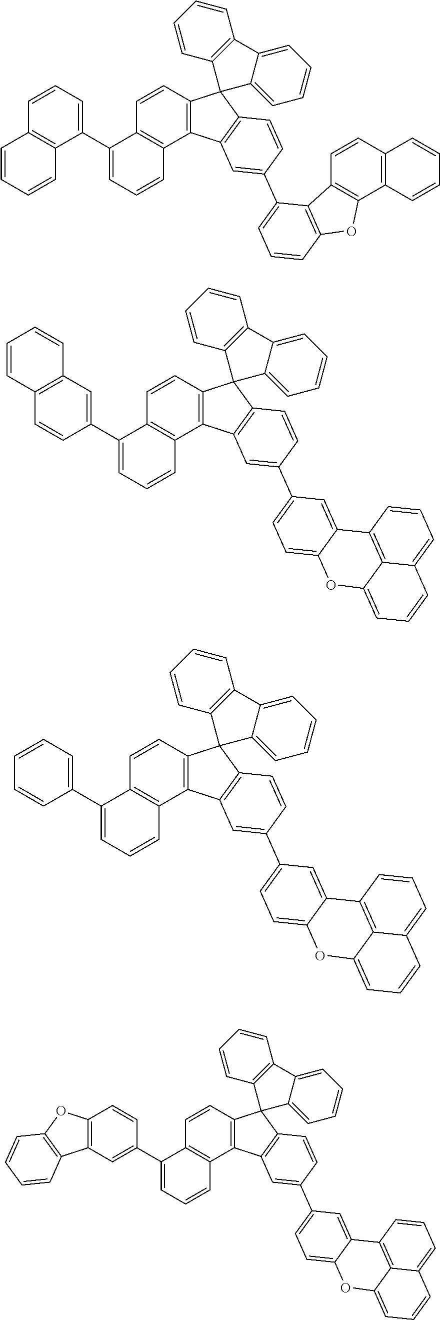 [SMILES: c1ccc2c(c1)-c1ccccc1C21c2ccc(-c3cccc4oc5c6ccccc6ccc5c34)cc2-c2c1ccc1c(-c3cccc4ccccc34)cccc21 ; c1ccc2c(c1)-c1ccccc1C21c2ccc(-c3ccc4c(c3)-c3cccc5cccc(c35)O4)cc2-c2c1ccc1c(-c3ccc4ccccc4c3)cccc21 ; c1ccc(-c2cccc3c4c(ccc23)C2(c3ccccc3-c3ccccc32)c2ccc(-c3ccc5c(c3)-c3cccc6cccc(c36)O5)cc2-4)cc1 ; c1ccc2c(c1)-c1ccccc1C21c2ccc(-c3ccc4c(c3)-c3cccc5cccc(c35)O4)cc2-c2c1ccc1c(-c3ccc4oc5ccccc5c4c3)cccc21]

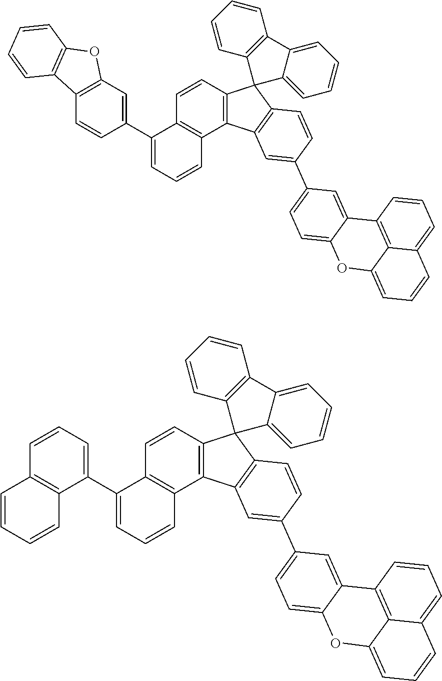 [SMILES: c1ccc2c(c1)-c1ccccc1C21c2ccc(-c3ccc4c(c3)-c3cccc5cccc(c35)O4)cc2-c2c1ccc1c(-c3ccc4c(c3)oc3ccccc34)cccc21 ; c1ccc2c(c1)-c1ccccc1C21c2ccc(-c3ccc4c(c3)-c3cccc5cccc(c35)O4)cc2-c2c1ccc1c(-c3cccc4ccccc34)cccc21]

The compound according to the exemplary embodiment has a fused fluorene structure in which a cyclic structure is fused with fluorene and the cyclic structure is further substituted at a particular position by a substituent Ry. This structure of the compound according to the embodiment suppresses interaction between molecules and aggregation of molecules. Therefore, luminous efficiency of an organic EL device is improved by using the compound according to the exemplary embodiment in the organic EL device. The compound according to the exemplary embodiment is suitably usable as a host material in the emitting layer of the organic EL device.

### Second Exemplary Embodiment

### Organic-Electroluminescence-Device Material

An organic-EL-device material according to the exemplary embodiment contains the compound according to the first exemplary embodiment. In an exemplary arrangement, the organic-EL-device material contains only the compound according to the first exemplary embodiment. In another exemplary arrangement, the organic-EL-device material contains the compound according to the first exemplary embodiment and a compound different from the compound according to the first exemplary embodiment.

In the organic-EL-device material according to the exemplary embodiment, the compound according to the first exemplary embodiment is preferably a host material. In this case, the organic-EL-device material may contain the compound according to the first exemplary embodiment as the host material and another compound such as a dopant material.

The compound according to the first exemplary embodiment is useful as the organic-EL-device material and useful as a material for an emitting layer of an organic EL device, especially, useful as a host material for a blue emitting layer.

### Third Exemplary Embodiment

### Organic Electroluminescence Device

An organic EL device according to a third exemplary embodiment will be described below.

The organic EL device according to the exemplary embodiment contains the compound according to the first exemplary embodiment as a first compound.

The organic EL device according to the exemplary embodiment includes an anode, a cathode, and an organic layer disposed between the anode and the cathode. The organic layer includes at least one layer formed from an organic compound(s). Alternatively, the organic layer includes a plurality of layers layered and formed from an organic compound(s). The organic layer may further contain an inorganic compound(s).

In the organic EL device according to the exemplary embodiment, at least one layer of the organic layer contains the first compound (compound according to the first exemplary embodiment).

In the organic EL device of the exemplary embodiment, at least one layer of the organic layer preferably includes an emitting region. In the organic EL device of the exemplary embodiment, the emitting region preferably includes at least one emitting layer. In an exemplary embodiment, the emitting layer contains a compound represented by the formula (1).

In the organic EL device of the exemplary embodiment, also preferably, the organic layer includes the emitting region, the emitting region includes a first emitting layer and a second emitting layer, and the first emitting layer contains the first compound as a first host material.

In a case where the emitting region includes the first emitting layer and the second emitting layer, the organic EL device according to the exemplary embodiment may also include, for instance, the anode, the first emitting layer, the second emitting layer, and the cathode in this order. Alternatively, for instance, the order of layering the first and second emitting layers may be reversed. Specifically, the organic EL device according to the exemplary embodiment may also include the anode, the second emitting layer, the first emitting layer, and the cathode in this order.

When the emitting region includes the first emitting layer and the second emitting layer, also preferably, the second emitting layer is provided between the anode and the cathode and the first emitting layer is provided between the anode and the second emitting layer in the organic EL device according to the exemplary embodiment.

When the emitting region includes the first emitting layer and the second emitting layer, also preferably, the first emitting layer is provided between the anode and the cathode and the second emitting layer is provided between the anode and the first emitting layer in the organic EL device according to the exemplary embodiment.

### Emission Wavelength of Organic EL Device

The organic EL device according to the exemplary embodiment preferably emits light having a maximum peak wavelength of 500 nm or less when being driven, more preferably emits light having a maximum peak wavelength in a range from 430 nm to 480 nm.

The maximum peak wavelength of the light emitted from the organic EL device when being driven is measured as follows. Voltage is applied on the organic EL device such that a current density becomes 10 mA/cm², where spectral radiance spectrum is measured by a spectroradiometer CS-2000 (manufactured by Konica Minolta, Inc.). A peak wavelength of an emission spectrum, at which the luminous intensity of the resultant spectral radiance spectrum is at the maximum, is measured and defined as the maximum peak wavelength (unit: nm).

A method of measuring the maximum peak wavelength of the compound herein is as follows. A toluene solution of a measurement target compound at a concentration ranging from 10⁻⁶ mol/L to 10⁻⁵ mol/L is prepared and put in a quartz cell. An emission spectrum (ordinate axis: luminous intensity, abscissa axis: wavelength) of the thus-obtained sample is measured at a normal temperature (300K). The emission spectrum can be measured using a spectrophotometer (machine name: F-7000) produced by Hitachi High-Tech Science Corporation. It should be noted that the apparatus for measuring the emission spectrum is not limited to the apparatus used herein.

A peak wavelength of the emission spectrum exhibiting the maximum luminous intensity is defined as the maximum peak wavelength. Herein, the maximum peak wavelength is occasionally referred to as a maximum fluorescence peak wavelength (FL-peak).

In the organic EL device according to the exemplary embodiment, the organic layer may consist of the emitting layer. Alternatively, the organic layer may further include, for instance, at least one layer selected from the group consisting of a hole injecting layer, a hole transporting layer, an electron injecting layer, an electron transporting layer, a hole blocking layer, and an electron blocking layer.

In the organic EL device according to the exemplary embodiment, the hole transporting layer is preferably provided between the anode and the emitting region.

In the organic EL device according to the exemplary embodiment, when the emitting region includes the first emitting layer and the second emitting layer that are layered in this order from a side close to the anode, the hole transporting layer is preferably provided between the anode and the first emitting layer. When the second emitting layer and the first emitting layer are layered in this order from the side close to the anode, the hole transporting layer is preferably provided between the anode and the second emitting layer.

In the organic EL device according to the exemplary embodiment, the electron transporting layer is preferably provided between the cathode and the emitting region.

In the organic EL device according to the exemplary embodiment, when the emitting region includes the first emitting layer and the second emitting layer that are layered in this order from a side close to the anode, the electron transporting layer is preferably provided between the cathode and the second emitting layer. When the second emitting layer and the first emitting layer are layered in this order from the side close to the anode, the electron transporting layer is preferably provided between the cathode and the first emitting layer.

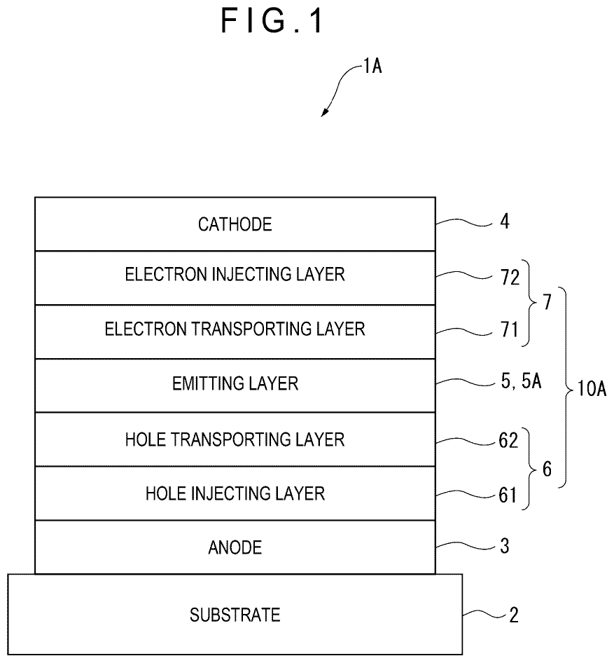

FIG. 1 schematically depicts an exemplary arrangement of the organic EL device according to the exemplary embodiment.

An organic EL device 1A depicted in FIG. 1 includes a substrate 2, an anode 3, a cathode 4, and an organic layer 10A disposed between the anode 3 and the cathode 4. The organic layer 10A includes a hole transporting zone 6, an emitting region 5A, and an electron transporting zone 7 in this order from a side close to the anode 3. The hole transporting zone 6 includes a hole injecting layer 61 and a hole transporting layer 62 in this order from the side close to the anode 3. The emitting region 5A includes a single emitting layer 5. The electron transporting zone 7 includes an electron transporting layer 71 and an electron injecting layer 72 in this order from a side close to the emitting region 5A.

### Emitting Layer

The emitting layer 5 contains a compound according to the first exemplary embodiment.

In the organic EL device 1A, the compound contained in the emitting layer 5 is preferably a compound represented by the formula (1).

### Luminescent Compound

In the organic EL device 1A, also preferably, the emitting layer 5 further contains a luminescent compound (preferably a fluorescent compound).

### Compound Represented by Formula (5)

In an exemplary arrangement of the organic EL device 1A, a luminescent compound contained in the emitting layer 5 is a compound represented by a formula (5) below.

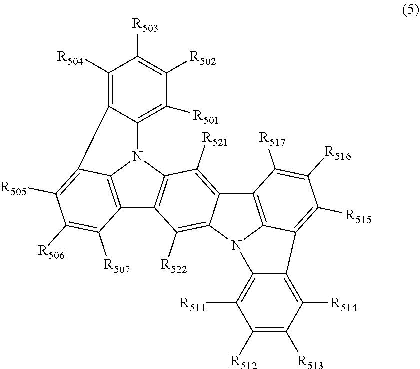

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded;
R₅₀₁ to R₅₀₇ and R₅₁₁ to R₅₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₅₂₁ and R₅₂₂ are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the luminescent compound, R₉₀₁, R₉₀₂, R₉₀₃, R₉₀₄, R₉₀₅, R₉₀₆ and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;

preferably, a substituted or unsubstituted alkyl group having 1 to 50 carbon atoms, or a substituted or unsubstituted aryl group having 6 to 50 ring carbon atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

“A combination of adjacent two or more of R₅₀₁ to R₅₀₇ and R₅₁₁ to R₅₁₇” refers to, for instance, a combination of R₅₀₁ and R₅₀₂, a combination of R₅₀₂ and R₅₀₃, a combination of R₅₀₃ and R₅₀₄, a combination of R₅₀₅ and R₅₀₆, a combination of R₅₀₆ and R₅₀₇, and a combination of R₅₀₁, R₅₀₂, and R₅₀₃.

In an exemplary embodiment, the compound represented by the formula (5) is a compound represented by a formula (52) below.

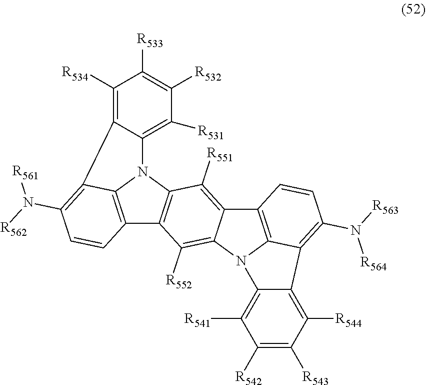

In the formula (52):
at least one combination of adjacent two or more of R₅₃₁ to R₅₃₄ and R₅₄₁ to
R₅₄₄ are mutually bonded to form a substituted or unsubstituted monocyclic ring, mutually bonded to form a substituted or unsubstituted fused ring, or not mutually bonded;
R₅₃₁ to R₅₃₄, R₅₄₁ to R₅₄₄, R₅₅₁, and R₅₅₂ forming neither the substituted or unsubstituted monocyclic ring nor the substituted or unsubstituted fused ring are each independently a hydrogen atom, a substituted or unsubstituted aryl group having 6 to 50 ring carbon atoms, or a substituted or unsubstituted heterocyclic group having 5 to 50 ring atoms; and
R₅₆₁ to R₅₆₄ are each independently a substituted or unsubstituted aryl group having 6 to 50 ring carbon atoms, or a substituted or unsubstituted heterocyclic group having 5 to 50 ring atoms.

### Compound Represented by Formula (6)

In an exemplary arrangement of the organic EL device 1A, the luminescent compound contained in the emitting layer 5 is preferably a compound represented by a formula (6) below.

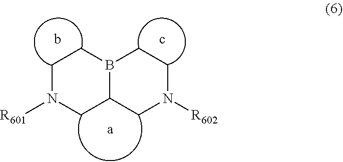

In the formula (6):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, the ring b or the ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In an exemplary arrangement of the organic EL device according to the exemplary embodiment, the ring a, ring b and ring c are each a ring (a substituted or unsubstituted aromatic hydrocarbon ring having 6 to 50 ring carbon atoms, or a substituted or unsubstituted heterocycle having 5 to 50 ring atoms) fused with a fused bicyclic structure formed of a boron atom and two nitrogen atoms at the center of the formula (6).

The “aromatic hydrocarbon ring” for the rings a, b, and c has the same structure as a compound formed by introducing a hydrogen atom to the “aryl group”.

Ring atoms of the “aromatic hydrocarbon ring” for the ring a include three carbon atoms on the fused bicyclic structure at the center of the formula (6).

Ring atoms of the “aromatic hydrocarbon ring” for the rings b and c include two carbon atoms on the fused bicyclic structure at the center of the formula (6).

Specific examples of the “substituted or unsubstituted aromatic hydrocarbon ring having 6 to 50 ring carbon atoms” include a compound formed by introducing a hydrogen atom to the “aryl group” described in the specific example group G1.

The “heterocycle” for the rings a, b, and c has the same structure as a compound formed by introducing a hydrogen atom to the “heterocyclic group” described above.

Ring atoms of the “heterocycle” for the ring a include three carbon atoms on the fused bicyclic structure at the center of the formula (6). Ring atoms of the “heterocycle” for the rings b and c include two carbon atoms on the fused bicyclic structure at the center of the formula (6). Specific examples of the “substituted or unsubstituted heterocycle having 5 to 50 ring atoms” include a compound formed by introducing a hydrogen atom to the “heterocyclic group” described in the specific example group G2.
R₆₀₁ and R₆₀₂ may be each independently bonded with the ring a, ring b, or ring c to form a substituted or unsubstituted heterocycle. The “heterocycle” in this arrangement includes a nitrogen atom on the fused bicyclic structure at the center of the formula (6). The heterocycle in the above arrangement optionally includes a hetero atom other than the nitrogen atom. R₆₀₁ and R₆₀₂ being bonded with the ring a, ring b, or ring c specifically means that atoms forming R₆₀₁ and R₆₀₂ are bonded with atoms forming the ring a, ring b, or ring c. For instance, R₆₀₁ may be bonded with the ring a to form a bicyclic (or tri- or -more cyclic) fused nitrogen-containing heterocycle, in which the ring including R₆₀₁ and the ring a are fused. Specific examples of the nitrogen-containing heterocycle include a compound corresponding to the nitrogen-containing bi(or-more) cyclic fused heterocyclic group in the specific example group G2.

The same applies to R₆₀₁ bonded with the ring b, R₆₀₂ bonded with the ring a, and R₆₀₂ bonded with the ring c.

Optionally, R₆₀₁ and R₆₀₂ are each independently not bonded with the ring a, ring b, or ring c.

In an exemplary embodiment, the ring a, ring b and ring c in the formula (6) are each independently a substituted or unsubstituted aromatic hydrocarbon ring having 6 to 50 ring carbon atoms.

In an exemplary embodiment, the ring a, ring b and ring c in the formula (6) are each independently a substituted or unsubstituted benzene ring or a substituted or unsubstituted naphthalene ring.

In an exemplary embodiment, R₆₀₁ and R₆₀₂ in the formula (6) are each independently a substituted or unsubstituted aryl group having 6 to 50 ring carbon atoms, or a substituted or unsubstituted heterocyclic group having 5 to 50 ring atoms, preferably, a substituted or unsubstituted aryl group having 6 to 50 ring carbon atoms.

In an exemplary embodiment, the compound represented by the formula (6) is a compound represented by a formula (62) below.

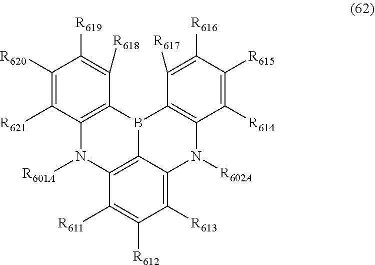

In the formula (62):
R_{601A} is bonded with at least one of R₆₁₁ or R₆₂₁ to form a substituted or unsubstituted heterocycle, or not bonded therewith to form no substituted or unsubstituted heterocycle;
R_{602A} is bonded with at least one of R₆₁₃ or R₆₁₄ to form a substituted or unsubstituted heterocycle, or not bonded therewith to form no substituted or unsubstituted heterocycle;
R_{601A} and R_{602A} not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
at least one combination of adjacent two or more of R₆₁₁ to R₆₂₁ are mutually bonded to form a substituted or unsubstituted monocyclic ring, mutually bonded to form a substituted or unsubstituted fused ring, or not mutually bonded; and
R₆₁₁ to R₆₂₁ not forming the substituted or unsubstituted heterocycle, not forming the monocyclic ring, and not forming the fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

In the formula (62), R₉₀₁, R₉₀₂, R₉₀₃, R₉₀₄, R₉₀₅, R₉₀₆, and R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.
R_{601A} and R_{602A} in the formula (62) are groups that respectively correspond to R₆₀₁ and R₆₀₂ in the formula (6).

For instance, R_{601A} and R₆₁₁ are optionally bonded with each other to form a bicyclic (or tri-or-more cyclic) fused nitrogen-containing heterocycle, in which the ring including R_{601A} and R₆₁₁ and a benzene ring corresponding to the ring a are fused. Specific examples of the nitrogen-containing heterocycle include a compound corresponding to the nitrogen-containing bi(or-more)cyclic fused heterocyclic group in the specific example group G2. The same applies to R_{601A} bonded with R₆₂₁, R_{602A} bonded with R₆₁₃, and R_{602A} bonded with R₆₁₄.

At least one combination of adjacent two or more of R₆₁₁ to R₆₂₁ are mutually bonded to form a substituted or unsubstituted monocyclic ring, or mutually bonded to form a substituted or unsubstituted fused ring.

For instance, R₆₁₁ and R₆₁₂ are optionally mutually bonded to form a structure in which a benzene ring, indole ring, pyrrole ring, benzofuran ring, benzothiophene ring or the like is fused to the six-membered ring bonded with R₆₁₁ and R₆₁₂, the resultant fused ring forming a naphthalene ring, carbazole ring, indole ring, dibenzofuran ring, or dibenzothiophene ring.

In an exemplary embodiment, the compound represented by the formula (6) is a compound represented by a formula (42-2) below.

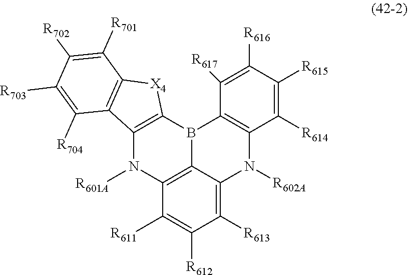

In the formula (42-2), R₆₁₁ to R₆₁₇, R_{601A} and R_{602A} each independently represent the same as R₆₁₁ to R₆₁₇, R_{601A} and R_{602A} in the formula (62);
X₄ is an oxygen atom or a sulfur atom;
at least one combination of adjacent two or more of R₇₀₁ to R₇₀₄ are mutually bonded to form a substituted or unsubstituted monocyclic ring, mutually bonded to form a substituted or unsubstituted fused ring, or not mutually bonded;
R₇₀₁ to R₇₀₄ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms; and
R₉₀₁, R₉₀₂, R₉₀₃, R₉₀₄, R₉₀₅, R₉₀₆, and R₉₀₇ in the formula (42-2) each independently represent the same as R₉₀₁, R₉₀₂, R₉₀₃, R₉₀₄, R₉₀₅, R₉₀₆, and R₉₀₇ in the formula (62).

### Compound Represented by Formula (3A)

In an exemplary arrangement of the organic EL device 1A, the luminescent compound contained in the emitting layer 5 is a compound represented by a formula (3A) below.

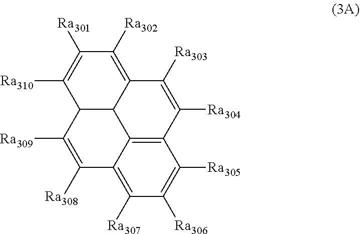

In the formula (3A):
at least one combination of adjacent two or more of Ra₃₀₁, Ra₃₀₂, Ra₃₀₃, Ra₃₀₄, Ra₃₀₅, Ra₃₀₆, Ra₃₀₇, Ra₃₀₈, Ra₃₀₉ and Ra₃₁₀ are mutually bonded to form a substituted or unsubstituted monocyclic ring, mutually bonded to form a substituted or unsubstituted fused ring, or not mutually bonded;
at least one of Ra₃₀₁ to Ra₃₁₀ is a monovalent group represented by a formula (31A) below; and
Ra₃₀₁ to Ra₃₁₀ forming neither the monocyclic ring nor the fused ring and not being the monovalent group represented by the formula (31A) are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

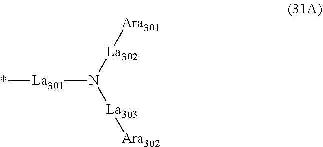

In the formula (31A):

Ara₃₀₁ and Ara₃₀₂ are each independently a substituted or unsubstituted aryl group having 6 to 50 ring carbon atoms, or a substituted or unsubstituted heterocyclic group having 5 to 50 ring atoms;

La₃₀₁, La₃₀₂, and La₃₀₃ are each independently a single bond, a substituted or unsubstituted arylene group having 6 to 30 ring carbon atoms, or a substituted or unsubstituted divalent heterocyclic group having 5 to 30 ring atoms, and
represents a bonding position to a pyrene ring in the formula (3A).

### Specific Example of Luminescent Compound

Specific examples of the luminescent compound are given below. It should however be noted that these specific examples are merely exemplary and the luminescent compound is not limited thereto.

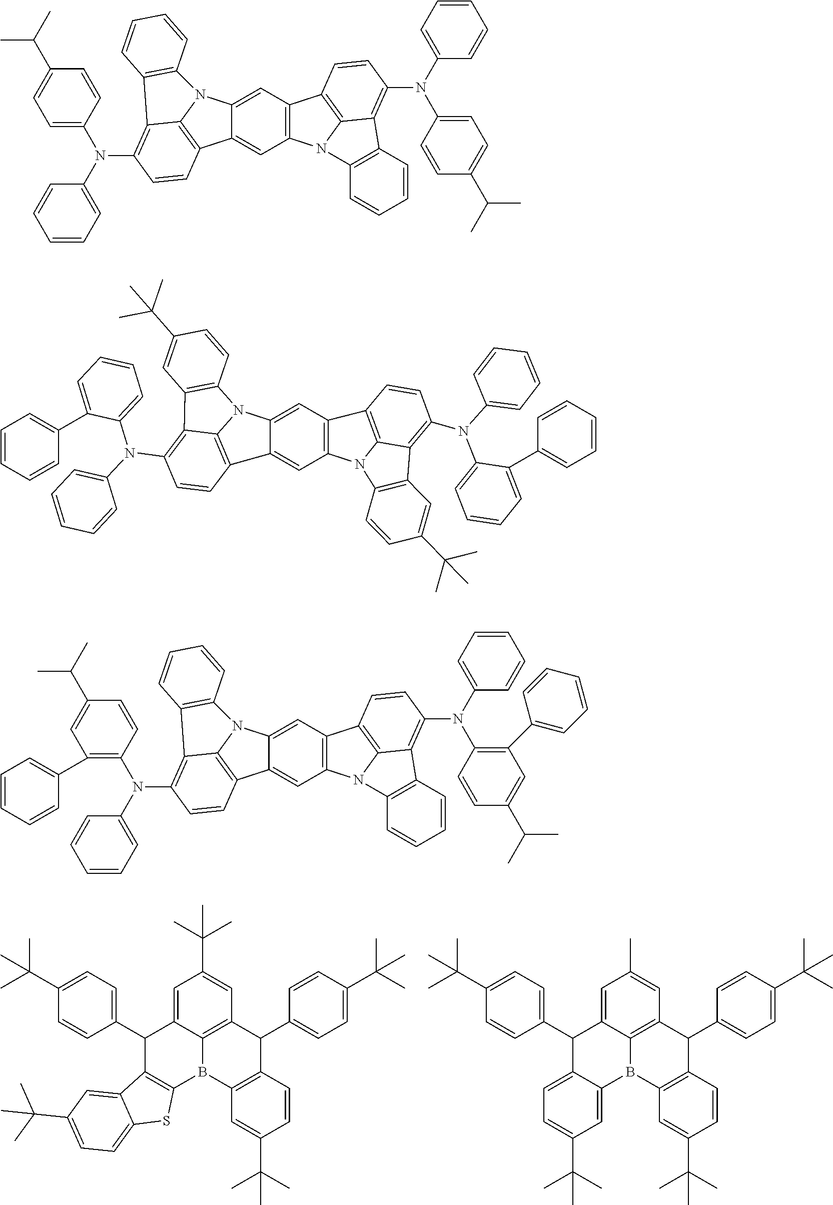

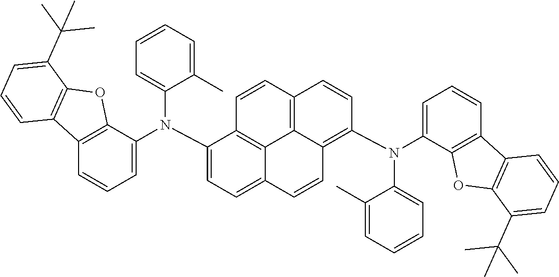 [SMILES: Cc1ccccc1N(c1ccc2ccc3c(N(c4ccccc4C)c4cccc5c4oc4c(C(C)(C)C)cccc45)ccc4ccc1c2c43)c1cccc2c1oc1c(C(C)(C)C)cccc12]

In the organic EL device 1A, the luminescent compound contained in the emitting layer 5 is preferably a compound that emits light having a maximum peak wavelength of 500 nm or less, more preferably a compound that emits light having a maximum peak wavelength in a range from 430 nm to 480 nm. In the organic EL device 1A, the luminescent compound contained in the emitting layer 5 is preferably a compound that emits fluorescence having a maximum peak wavelength of 500 nm or less, more preferably a compound that emits fluorescence having a maximum peak wavelength in a range from 430 nm to 480 nm.

When the emitting layer 5 of the organic EL device 1A contains a compound according to the first exemplary embodiment and a luminescent compound, the first compound according to the first exemplary embodiment is preferably a host material (occasionally referred to as a matrix material) and the luminescent compound is preferably a dopant material (occasionally referred to as a guest material, emitter, or luminescent material).

Herein, the “host material” refers to, for instance, a material that accounts for “50 mass % or more of the layer”. That is, for instance, the emitting layer 5 contains 50 mass % or more of a compound represented by the formula (1A) with respect to the total mass of the emitting layer in the organic EL device 1A.

### Film Thickness of Emitting Layer

A film thickness of the emitting layer 5 is preferably in a range from 5 nm to 50 nm, more preferably in a range from 7 nm to 50 nm, and still more preferably in a range from 10 nm to 50 nm. A film thickness of the emitting layer of 5 nm or more facilitates the formation of the emitting layer and the adjustment of chromaticity. A film thickness of the emitting layer of 50 nm or less easily inhibits an increase in drive voltage.

### Content Ratios of Compounds in Emitting Layer

When the emitting layer 5 contains a compound according to the first exemplary embodiment and a luminescent compound, content ratios of the compound according to the first exemplary embodiment and the luminescent compound in the emitting layer 5 preferably fall, for instance, within ranges below.

The content ratio of the compound according to the first exemplary embodiment falls within, preferably, in a range from 80 mass % to 99 mass %, more preferably in a range from 90 mass % to 99 mass %, and still more preferably in a range from 95 mass % to 99 mass %.

The content ratio of the luminescent compound falls within, preferably, in a range from 1 mass % to 10 mass %, more preferably in a range from 1 mass % to 7 mass %, and still more preferably in a range from 1 mass % to 5 mass %.

The upper limit of the total of the content ratios of the compound according to the first exemplary embodiment and the luminescent compound in the emitting layer 5 is 100 mass %.

In the exemplary embodiment, the emitting layer 5 may further contain any other material than the compound according to the first exemplary embodiment and the luminescent compound.

The emitting layer 5 may contain a single type or two or more types of compound according to the first exemplary embodiment. The emitting layer 5 may contain a single type of luminescent compound or two or more types of luminescent compounds.

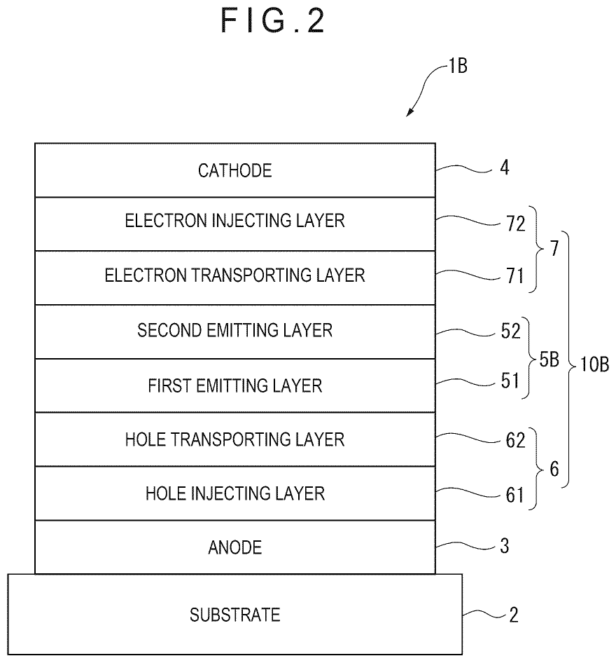

FIG. 2 schematically depicts another exemplary arrangement of the organic EL device according to the exemplary embodiment.

An organic EL device 1B depicted in FIG. 2 is different from the organic EL device 1A in that an organic layer 10B includes a first emitting region 5B, and the rest of components and arrangements of the organic EL device 1B are the same as those of the organic EL device 1A. The first emitting region 5B includes a first emitting layer 51 and a second emitting layer 52 in this order from a side close to the anode 3.

The first emitting layer 51 contains a first compound and the second emitting layer 52 contains a second compound.

### First Compound

In the organic EL device 1B, the first compound is a compound according to the first exemplary embodiment.

In the organic EL device according to the exemplary embodiment, the first compound is preferably a compound represented by the formula (1).

### Second Compound

In the organic EL device 1B, the second compound, which is not particularly limited to, is exemplified by a second compound represented by a formula (2) below.

In an exemplary arrangement of the organic EL device 1B, the second compound is a compound represented by the formula (2) below.

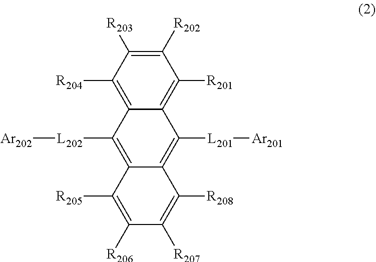

In the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O) R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms, and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the second compound, R₉₀₁, R₉₀₂, R₉₀₃, R₉₀₄, R₉₀₅, R₉₀₆, R₉₀₇, R₈₀₁ and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In an exemplary arrangement of the organic EL device 1B, the second compound has at least one group represented by a formula (HY1) below in a molecule.

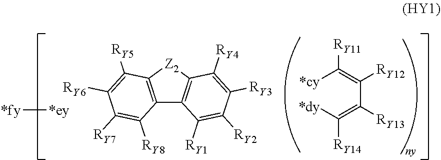

In the formula (HY1):
R_{Y1} to R_{Y8} and R_{Y1} to R_{Y14} are each independently a hydrogen atom, a halogen atom, a cyano group, a nitro group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aryl group having 6 to 50 ring carbon atoms, or a substituted or unsubstituted heterocyclic group having 5 to 50 ring atoms;
ny is 0 or 1;
when ny is 0, one of RY to R_{Y8} is a single bond with *ey;
when ny is 1, one of R_{Y1} and R_{Y2}, one of R_{Y2} and R_{Y3}, or one of R_{Y3} and R_{Y4} is a single bond with *cy while the other of R_{Y1} and R_{Y2}, the other of R_{Y2} and R_{Y3}, or the other of R_{Y3} and R_{Y4} is a single bond with *dy, and one of R_{Y5} to R_{Y8}, R_{Y11} to R_{Y14}, and R_{Y1} to R_{Y4} not being the single bonds with *cy and *dy is a single bond with *ey;
Z₂ is an oxygen atom or a sulfur atom; and
*fy represents a bonding position to an atom in the second compound.

In an exemplary arrangement of the organic EL device 1B, when ny is 0 in the formula (HY1), the group represented by the formula (HY1) is represented by a formula (HY10) below.

In an exemplary arrangement of the organic EL device 1B, the second compound has at least one group represented by the formula (HY10) below in a molecule.

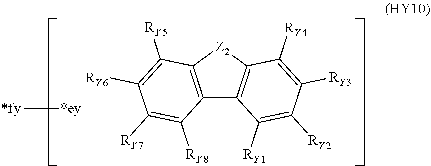

In the formula (HY10): Rvi to R_{Y8} and Z₂ respectively represent the same as R_{Y1} to R_{Y8} and Z₂ in the formula (HY1);
one of R_{Y1} to R_{Y8} is a single bond with *ey; and
*fy represents a bonding position to an atom in the second compound.

In an exemplary arrangement of the organic EL device 1B, the second compound has, in a molecule, at least one group represented by the formula (HY1) where ny is 1.

In an exemplary arrangement of the organic EL device 1B, the second compound has, in a molecule, at least one group selected from the group consisting of groups represented by formulae (HY11), (HY12) and (HY13) below.

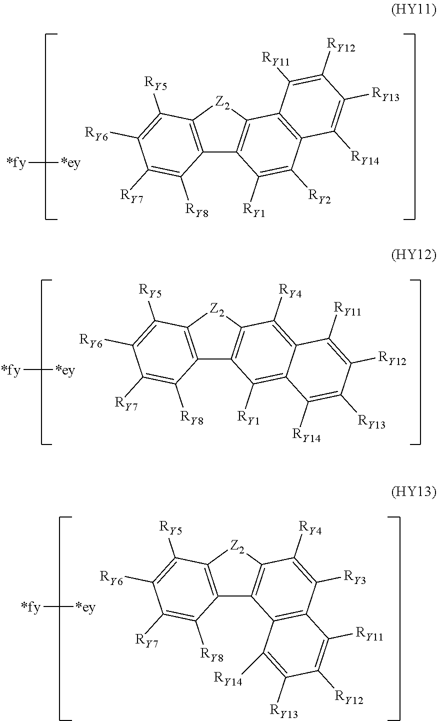

In the formulae (HY11), (HY12) and (HY13): R_{Y1} to R_{Y8}, R_{Y11} to R_{Y14}, and Z₂ respectively represent the same as R_{Y1} to R_{Y8}, R_{Y11} to R_{Y14}, and Z₂ in the formula (HY1);
one of R_{Y1} to R_{Y8} and R_{Y11} to R_{Y14} is a single bond with *ey; and
*fy represents a bonding position to an atom in the second compound.

In an exemplary arrangement of the organic EL device 1B, the second compound is represented by the formula (2) and has, in a molecule, at least one group represented by the formula (HY1).

In an exemplary arrangement of the organic EL device 1B, at least one of Ar₂₀₁ or Ar₂₀₂ in the formula (2) is a group represented by the formula (HY1).

In an exemplary arrangement of the organic EL device 1B, Ar₂₀₁ or Ar₂₀₂ in the formula (2) is a group represented by the formula (HY1).

In an exemplary arrangement of the organic EL device 1B, the second compound is represented by the formula (2) and has, in a molecule, at least one group selected from the group consisting of the groups represented by the formulae (HY11), (HY12) and (HY13).

In an exemplary arrangement of the organic EL device 1B, at least one of Ar₂₀₁ or Ar₂₀₂ in the formula (2) is any one group selected from the group consisting of the groups represented by the formulae (HY11), (HY12) and (HY13).

In an exemplary arrangement of the organic EL device 1B, Ar₂₀₁ or Ar₂₀₂ in the formula (2) is any one group selected from the group consisting of the groups represented by the formulae (HY11), (HY12) and (HY13).

In an exemplary arrangement of the organic EL device 1B, the second compound has, in a molecule, at least one group selected from the group consisting of the groups represented by the formulae (HY1), (HY10), (HY11), (HY12) and (HY13), thereby improving excitation resistance of the second compound. Use of the second compound described above in the second emitting layer makes it easy to prolong a lifetime of the organic EL device.

In an exemplary arrangement of the organic EL device 1B, the groups specified to be “substituted or unsubstituted” in the second compound are each an “unsubstituted” group.

### Method of Producing Second Compound

The second compound according to the exemplary embodiment is producible by a known method or through a known alternative reaction using a known material(s) tailored for the target compound in accordance with the known method.

### Specific Examples of Second Compound

Specific examples of the second compound according to the exemplary embodiment include the following compounds. However, the invention is by no means limited to the specific examples. Herein, a deuterium atom is denoted as D in formulae, and a protium atom is denoted as H or a description of a protium atom is omitted.

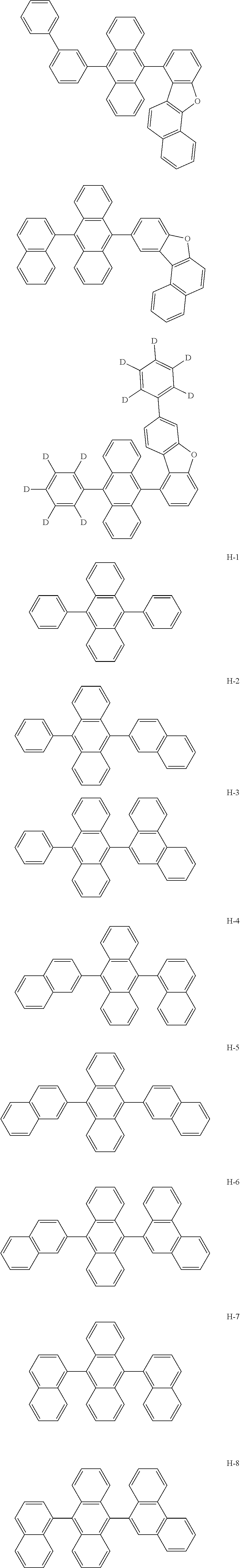

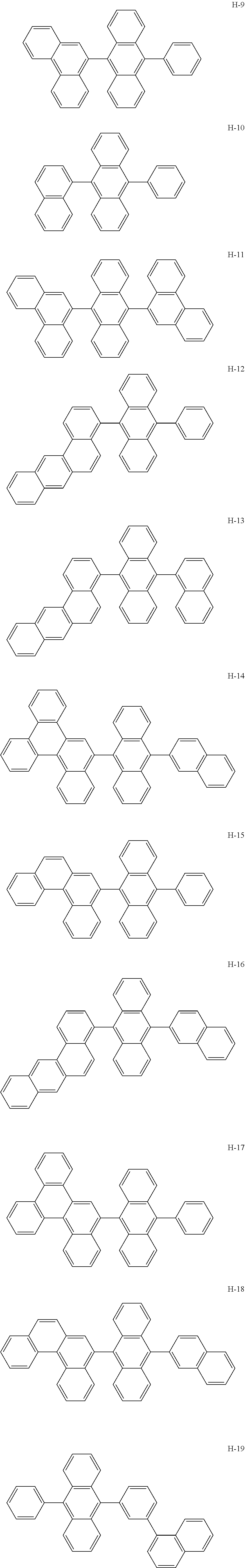

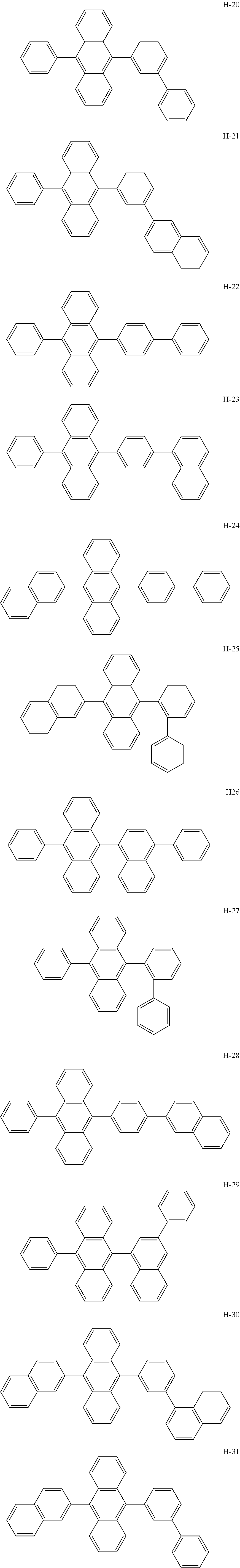

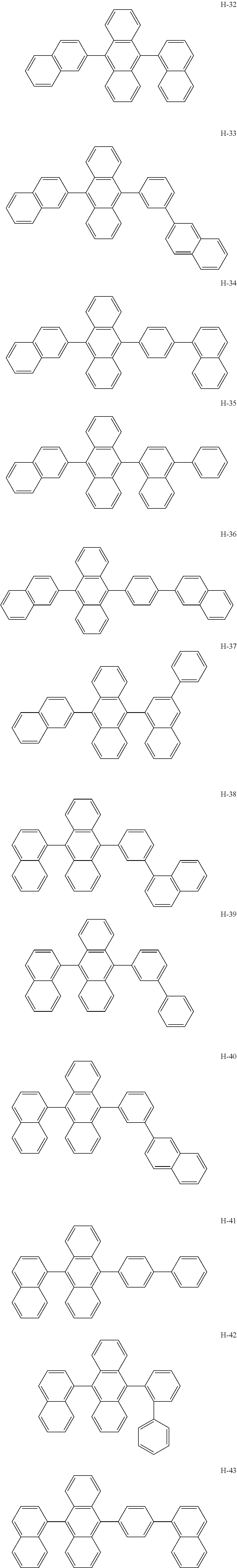

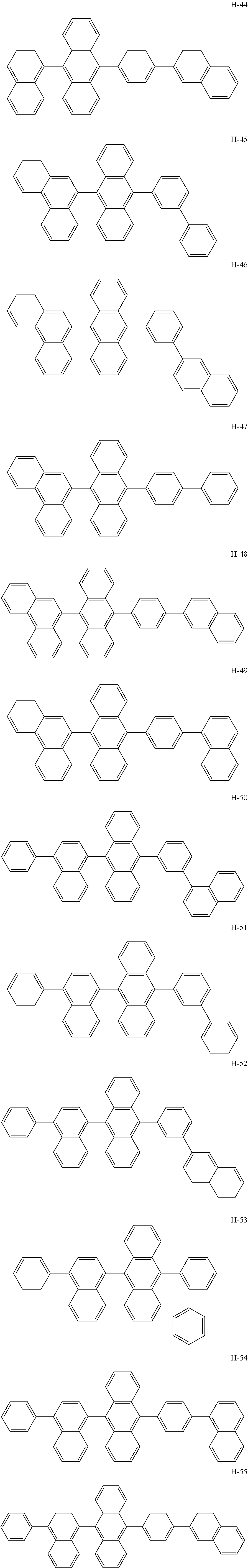

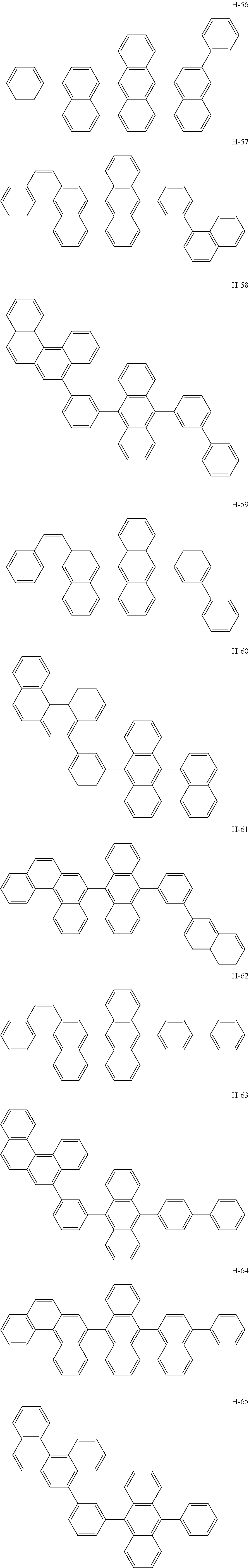

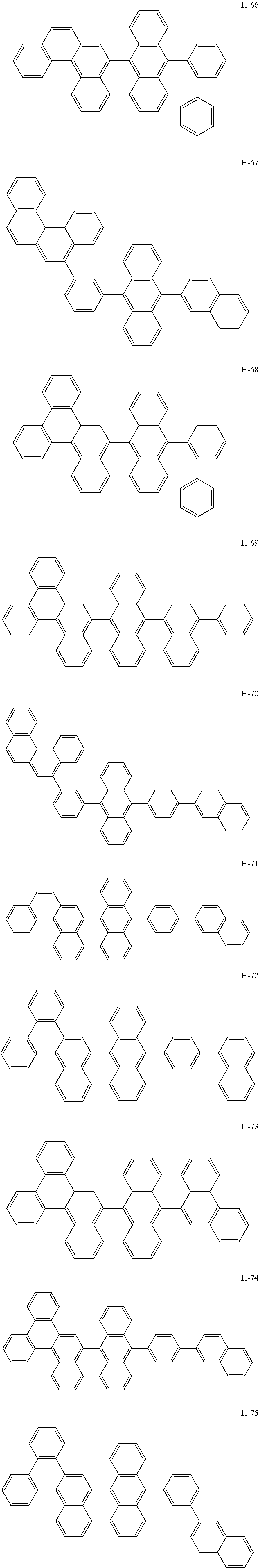

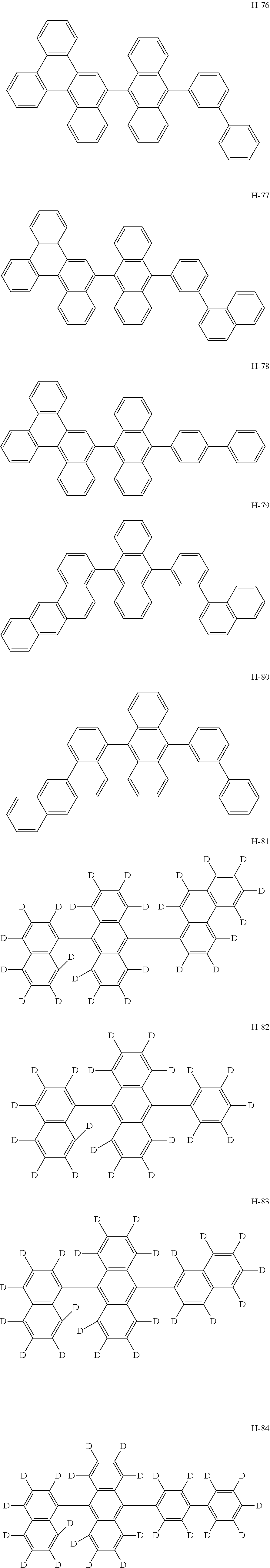

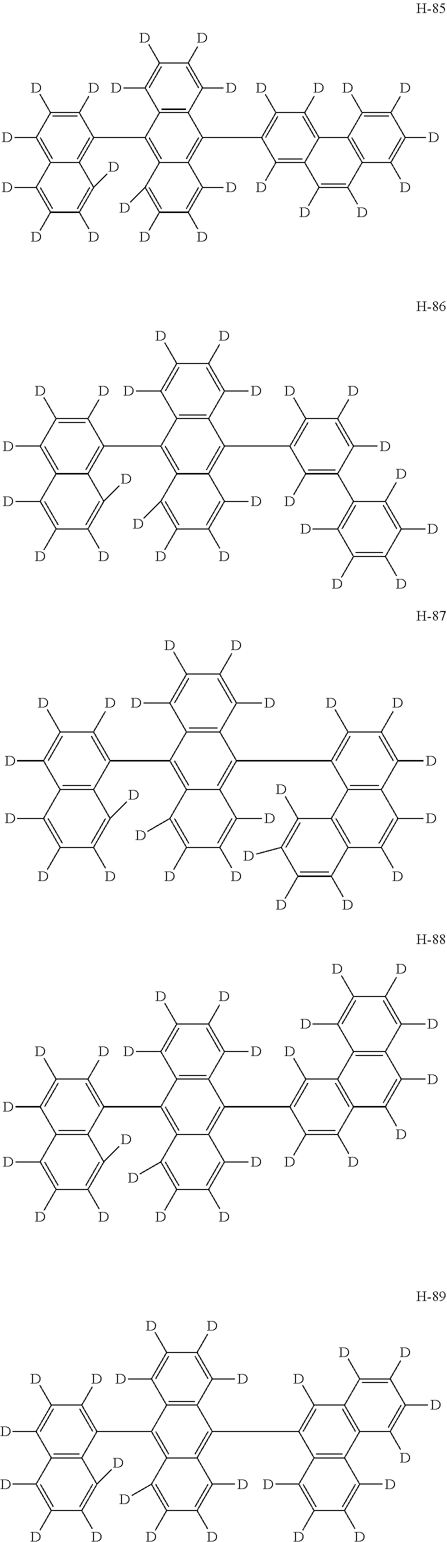

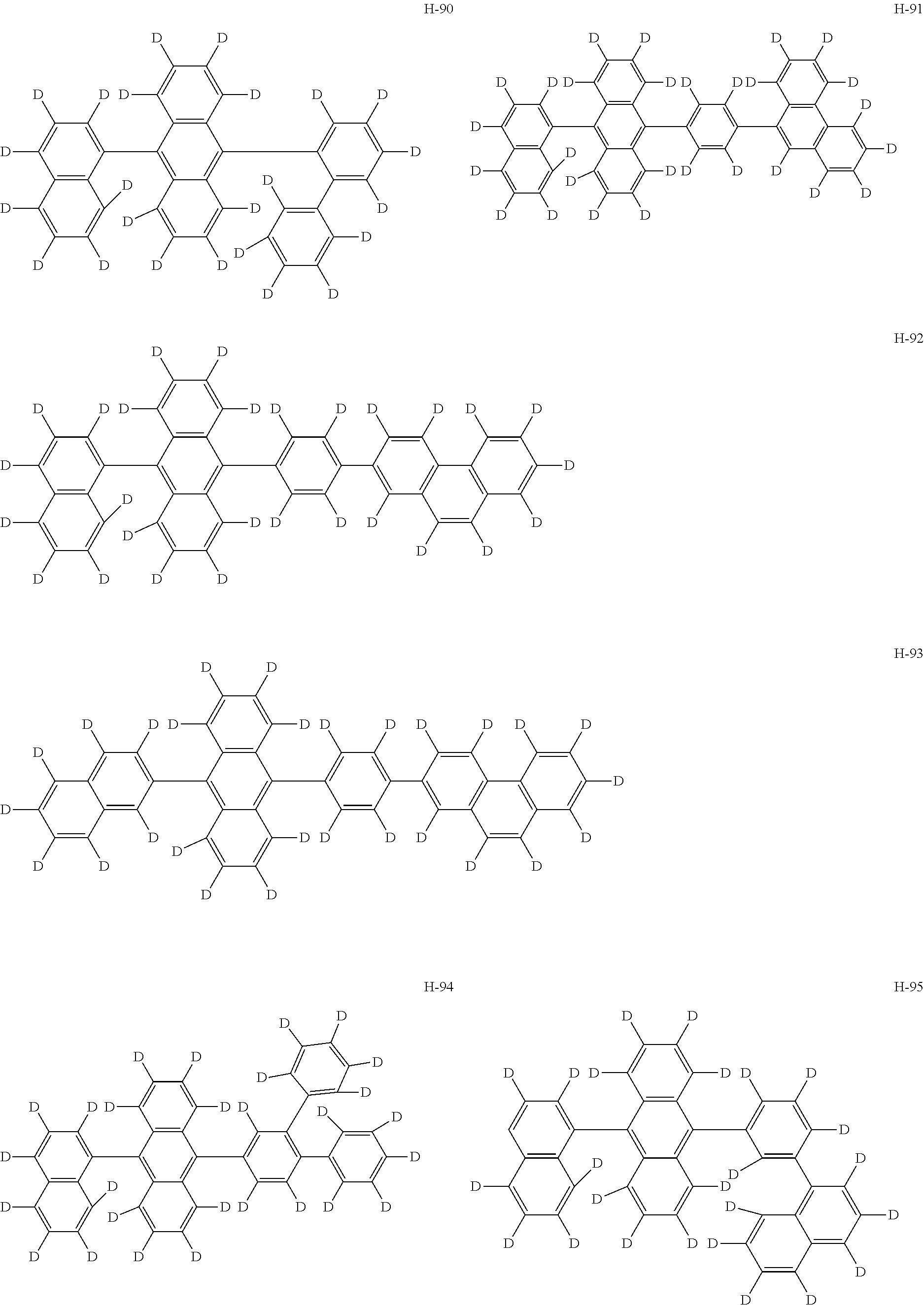

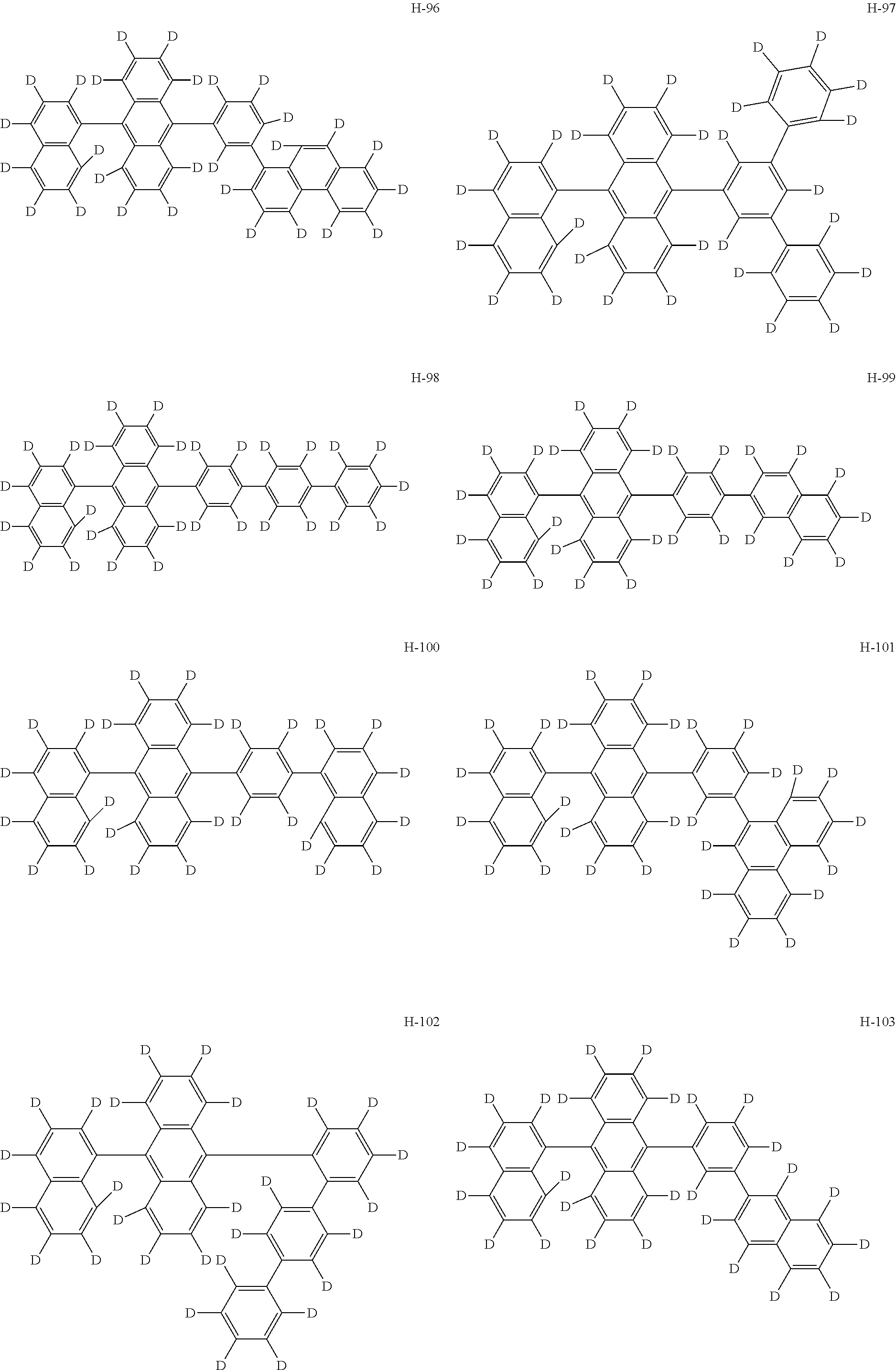

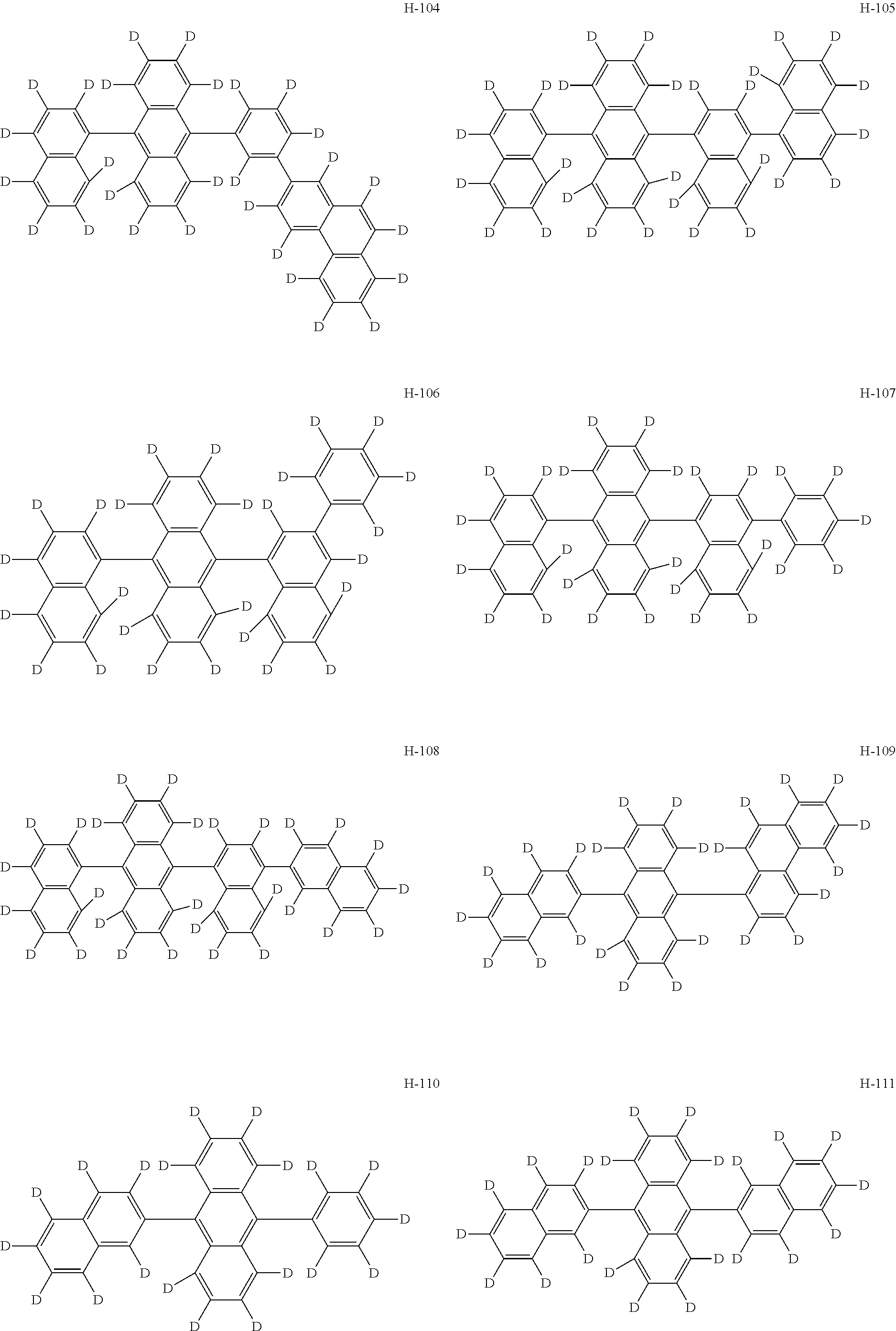

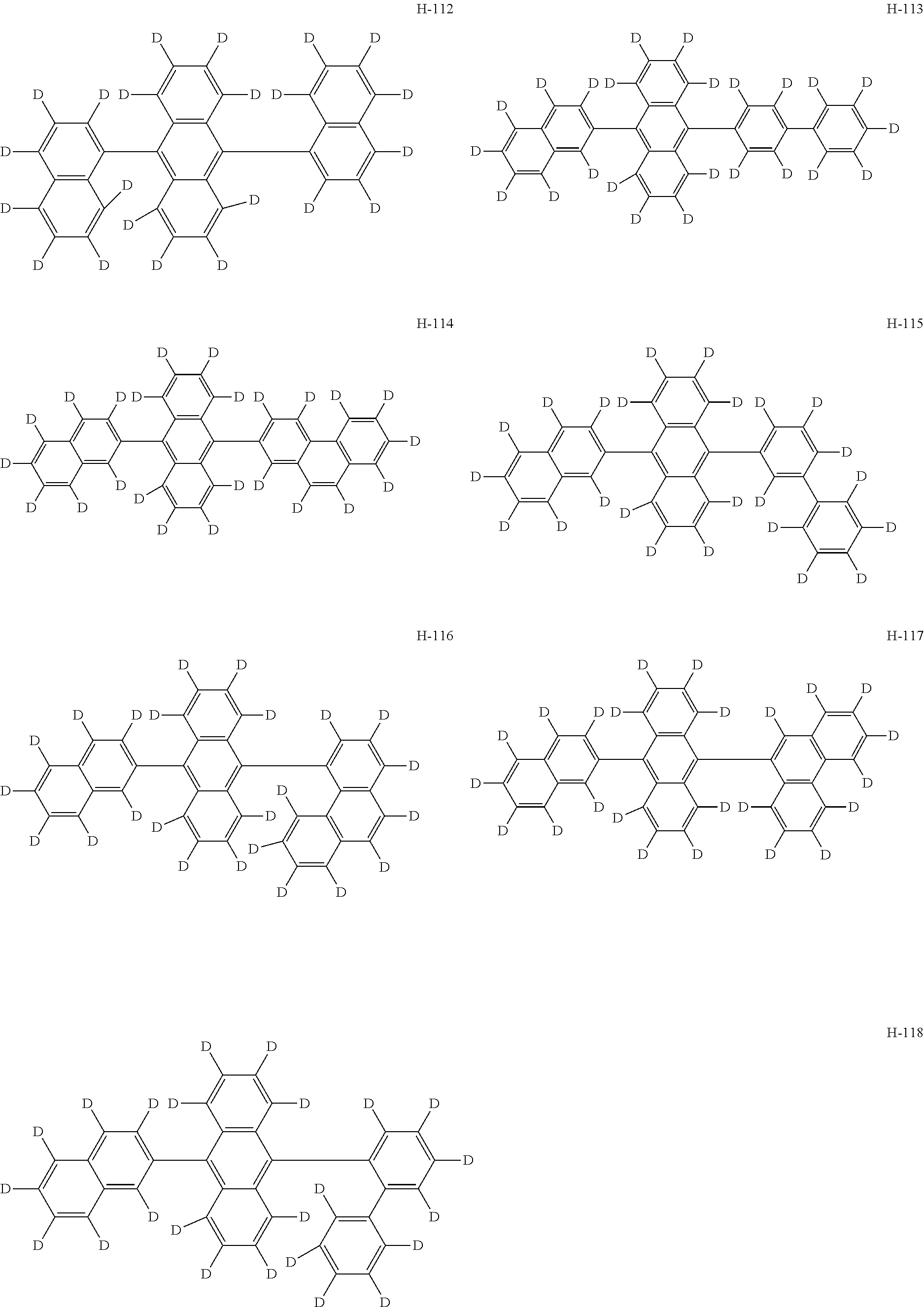

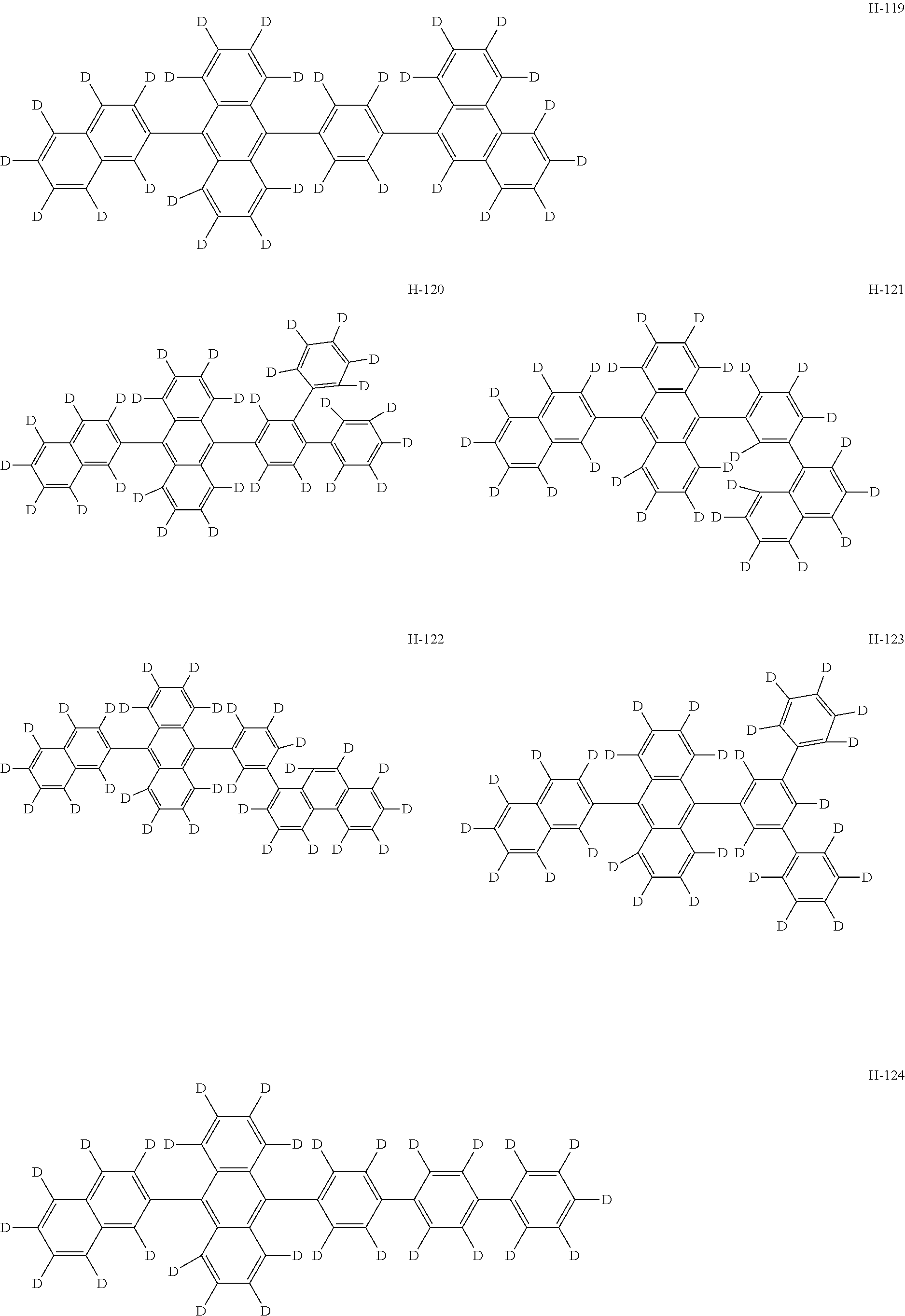

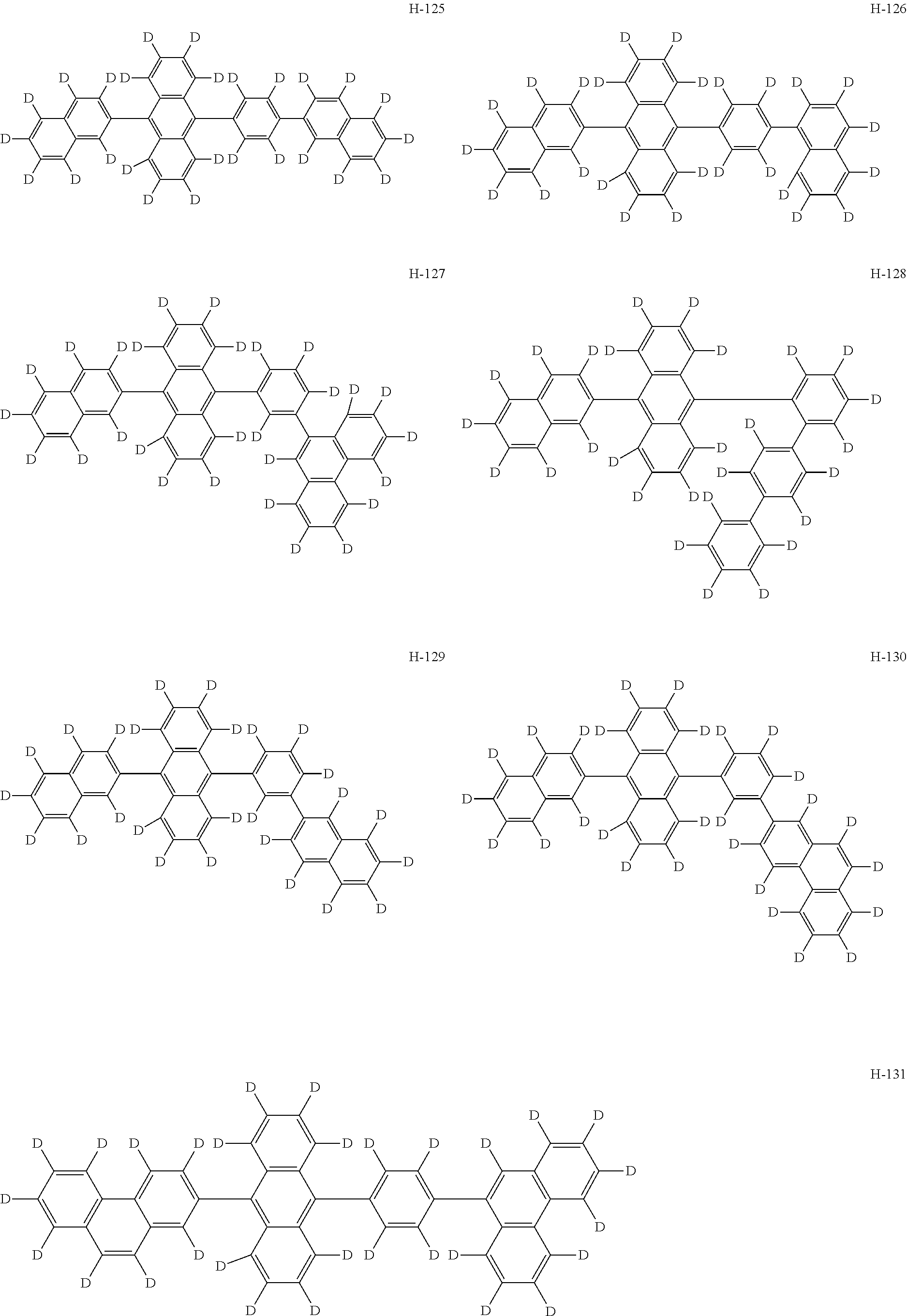

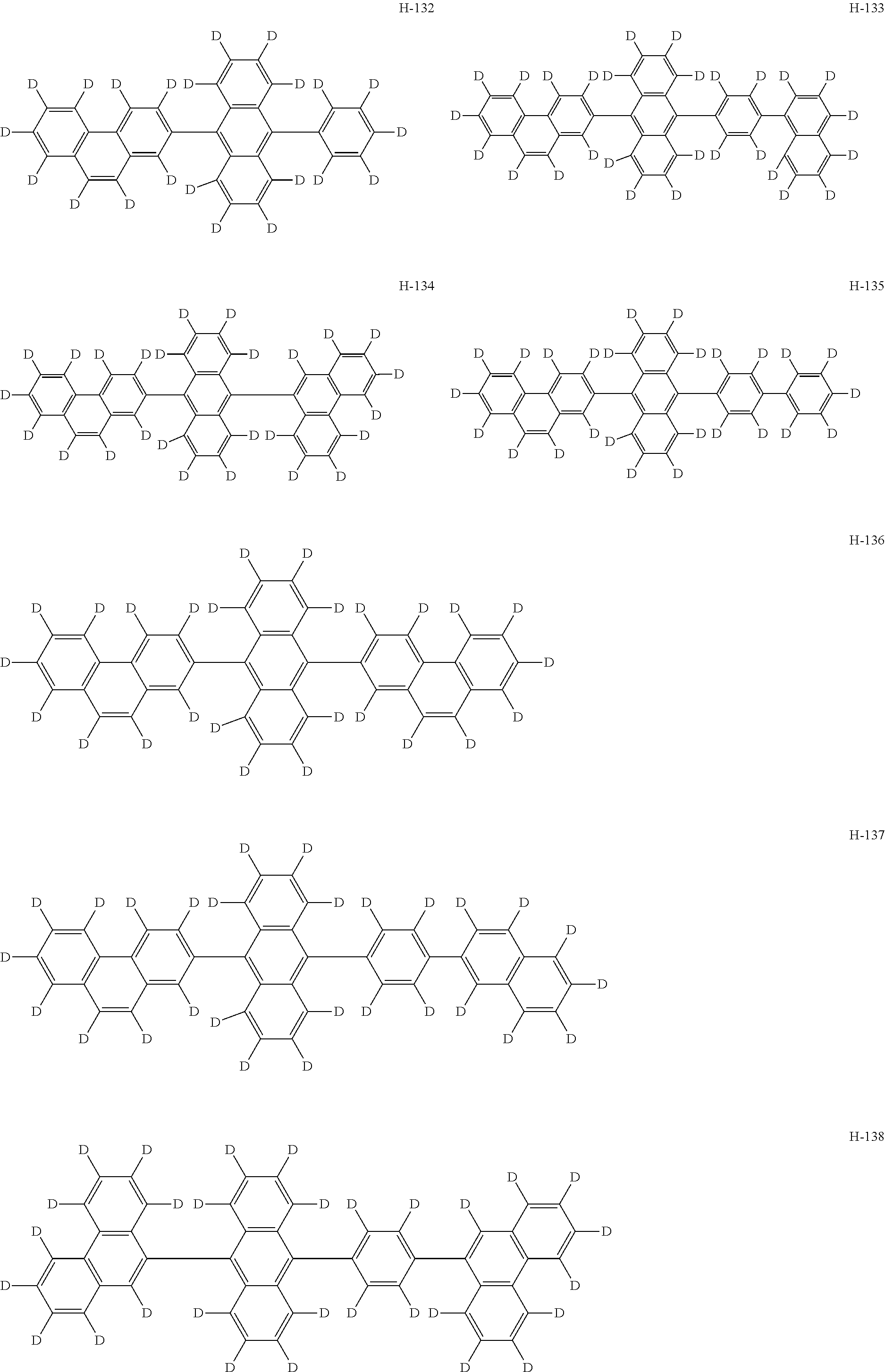

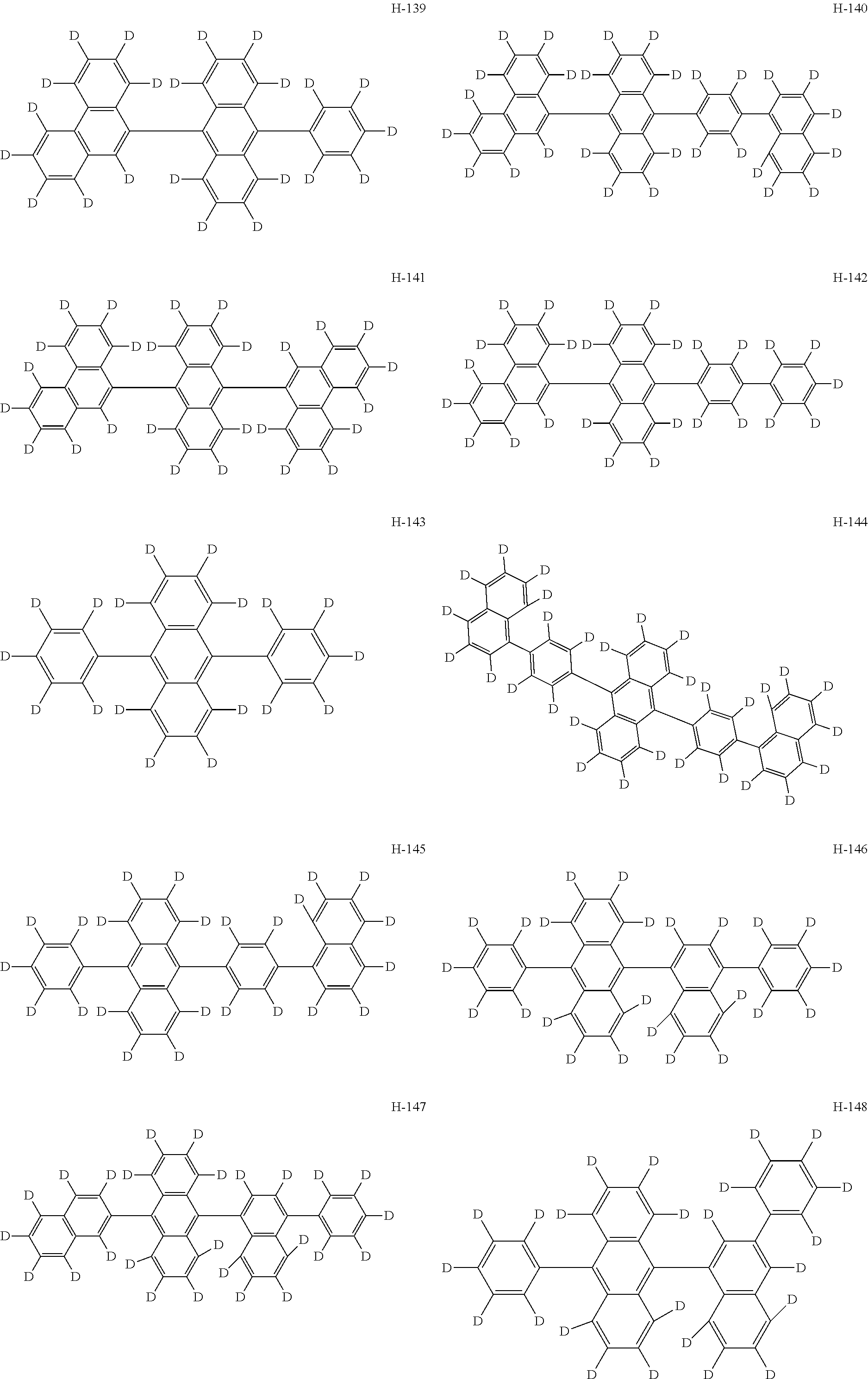

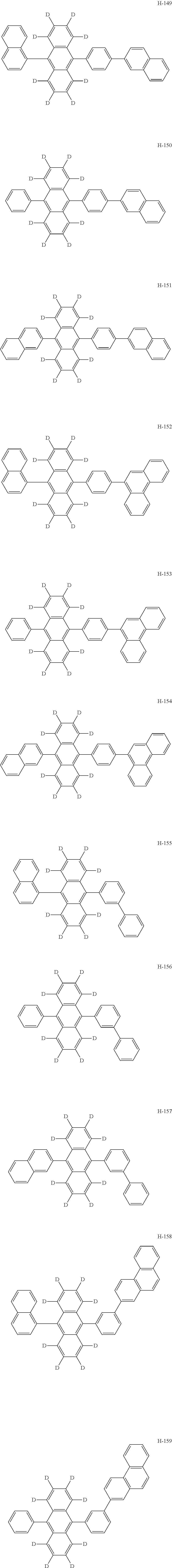

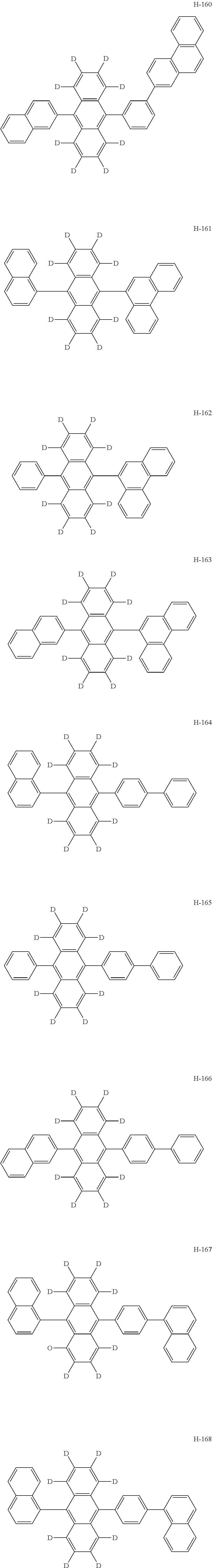

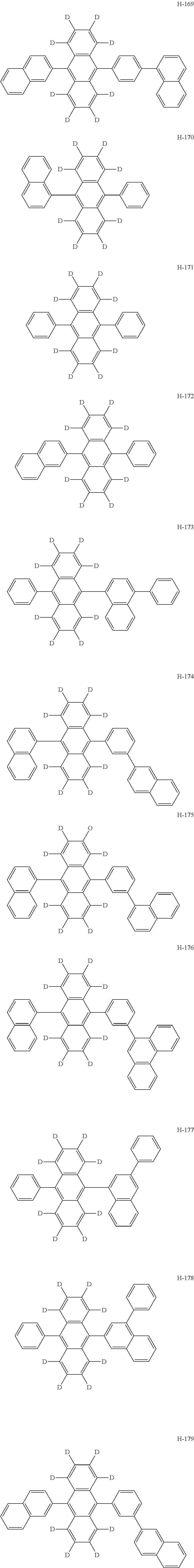

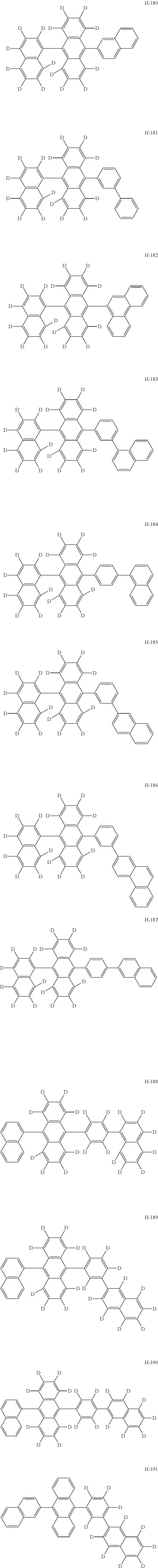

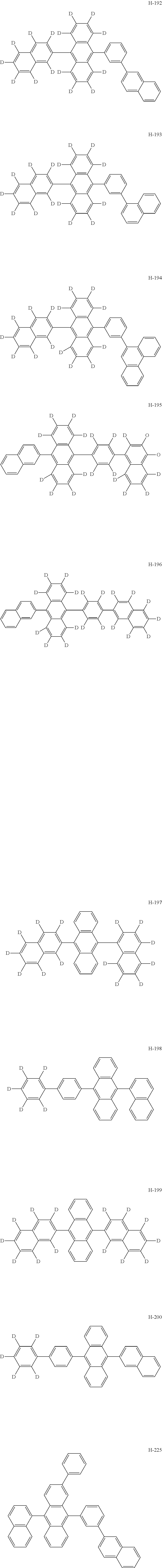

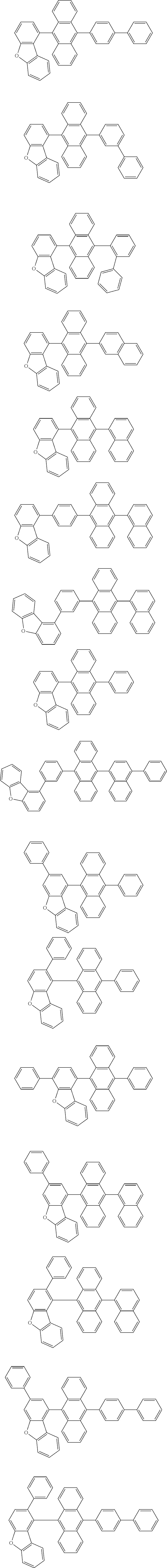

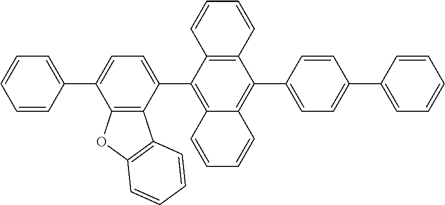 [SMILES: c1ccc(-c2ccc(-c3c4ccccc4c(-c4ccc(-c5ccccc5)c5oc6ccccc6c45)c4ccccc34)cc2)cc1]

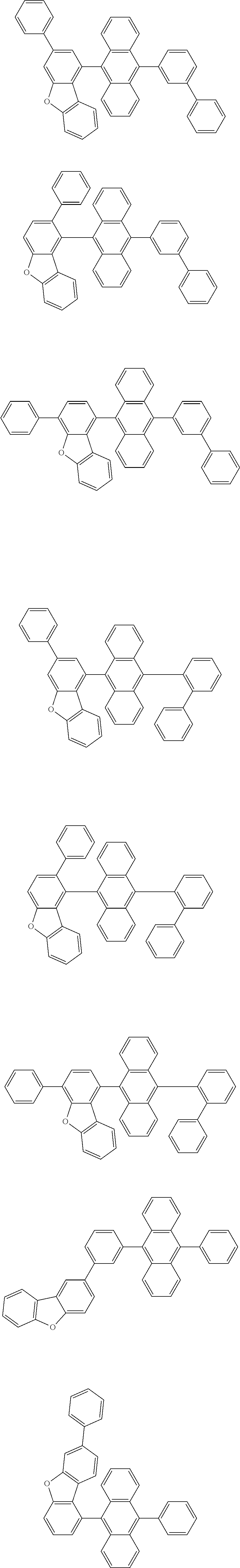

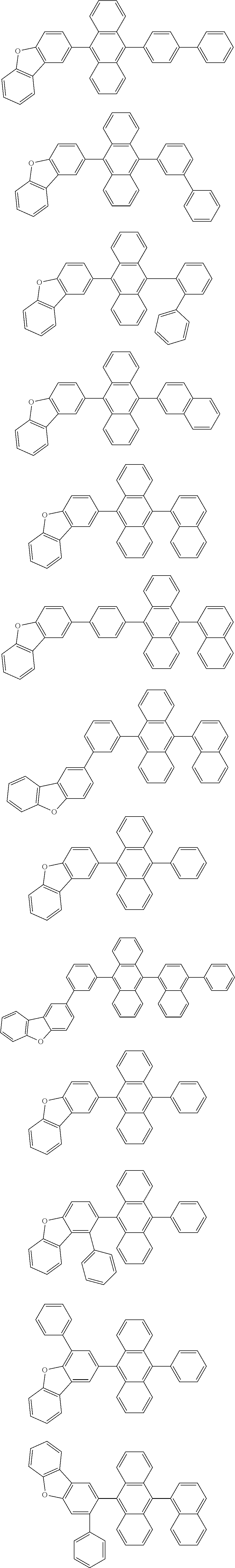

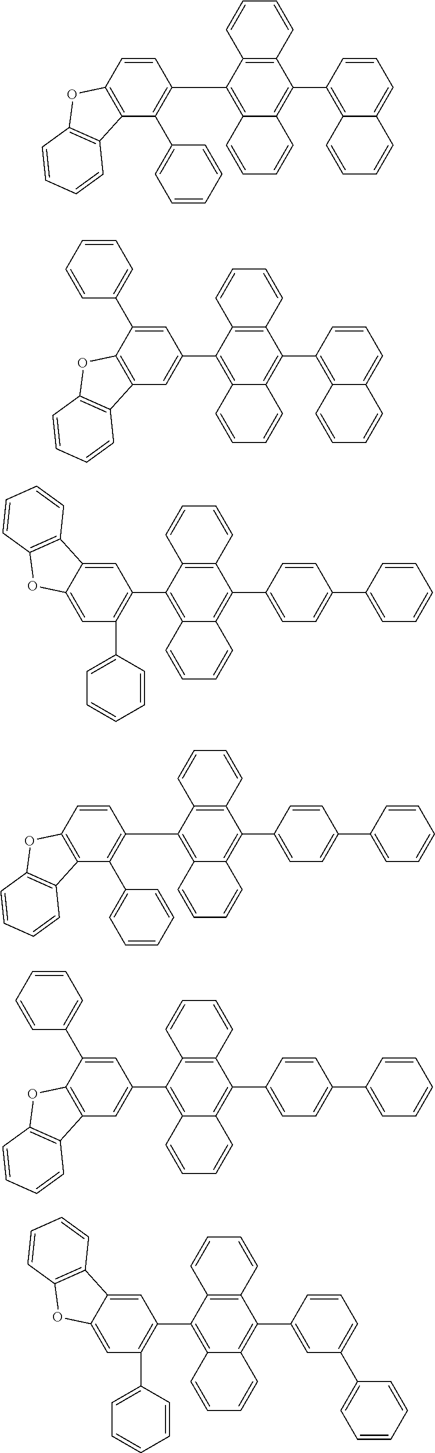

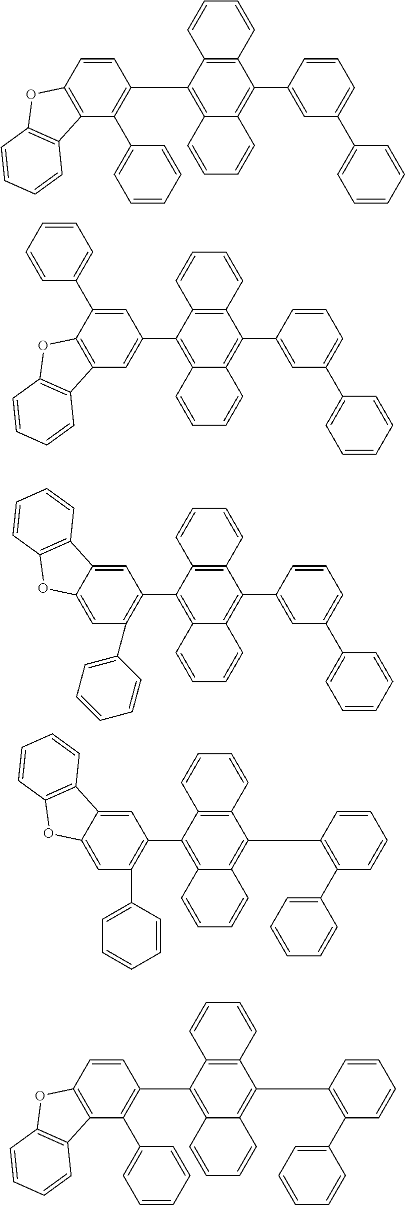

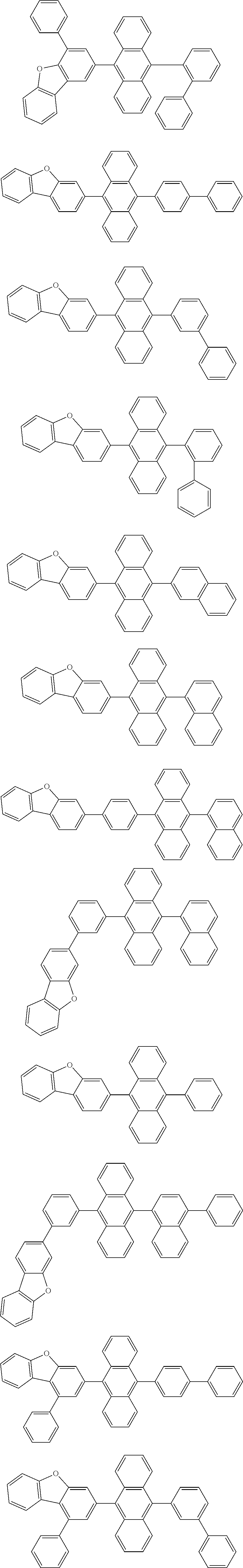

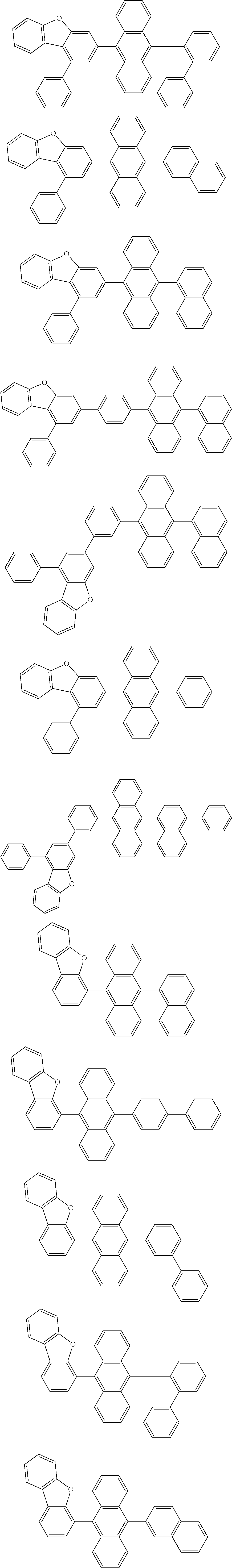

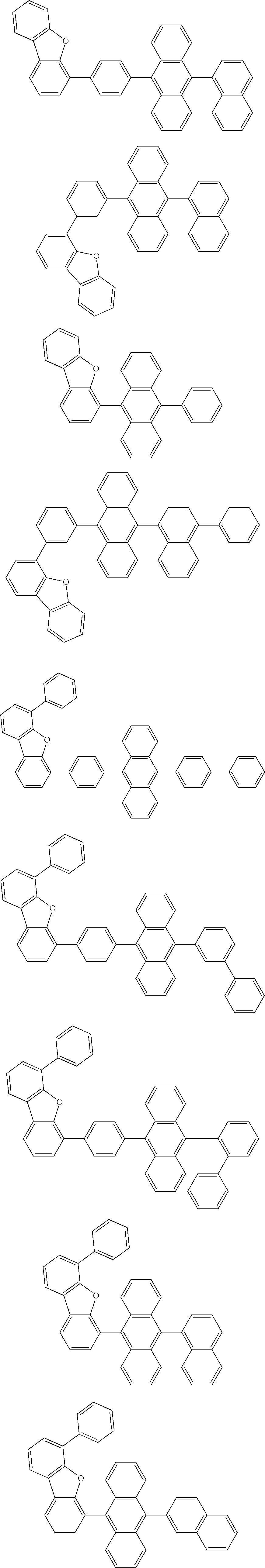

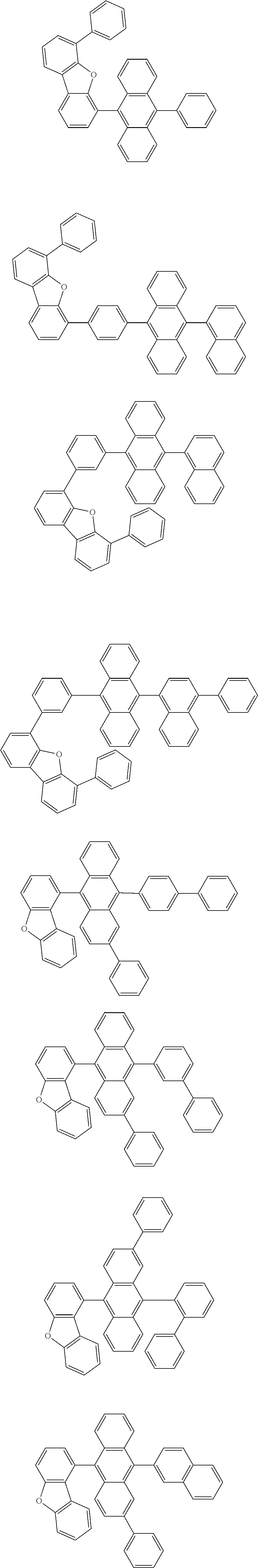

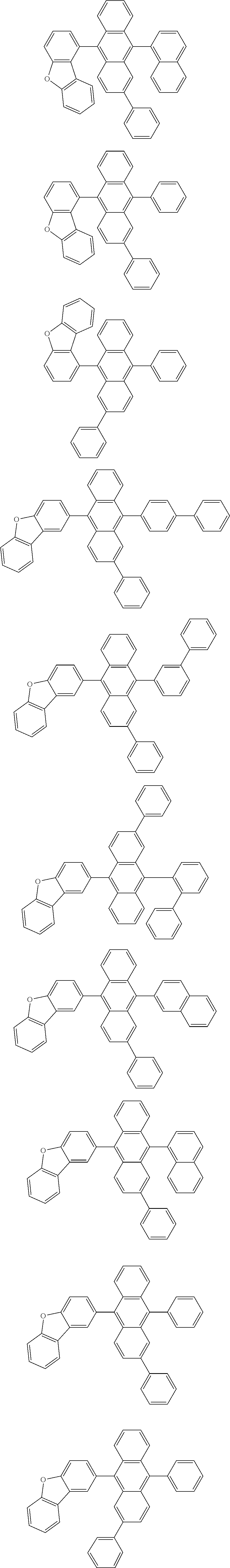

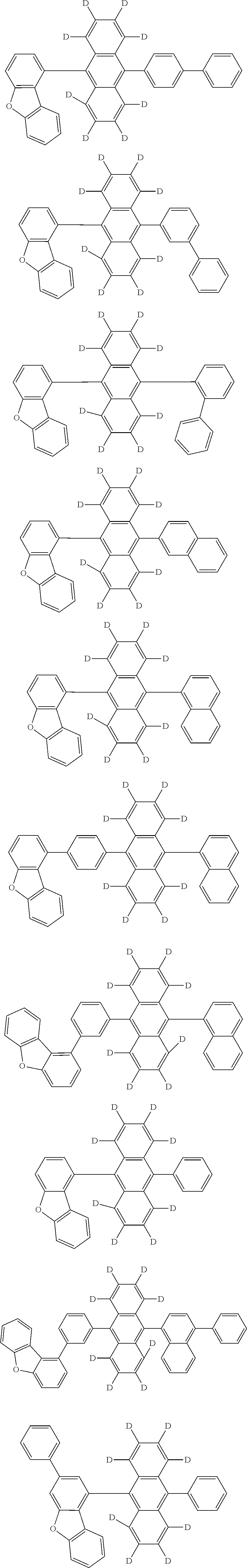

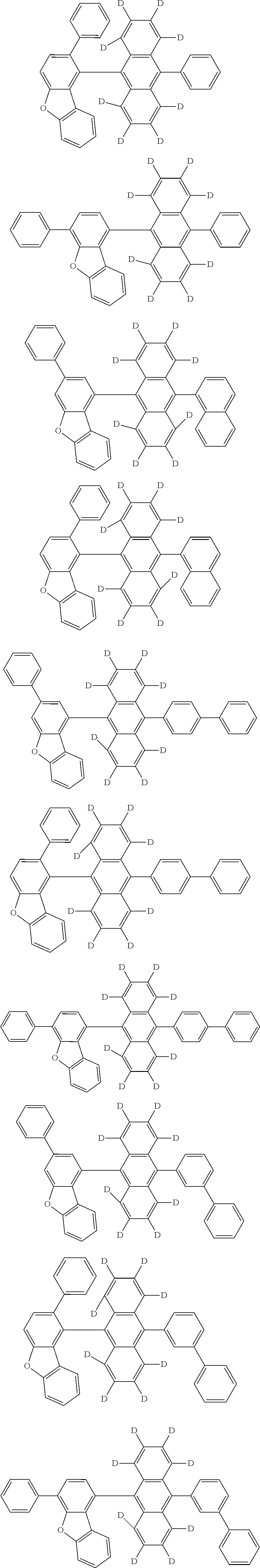

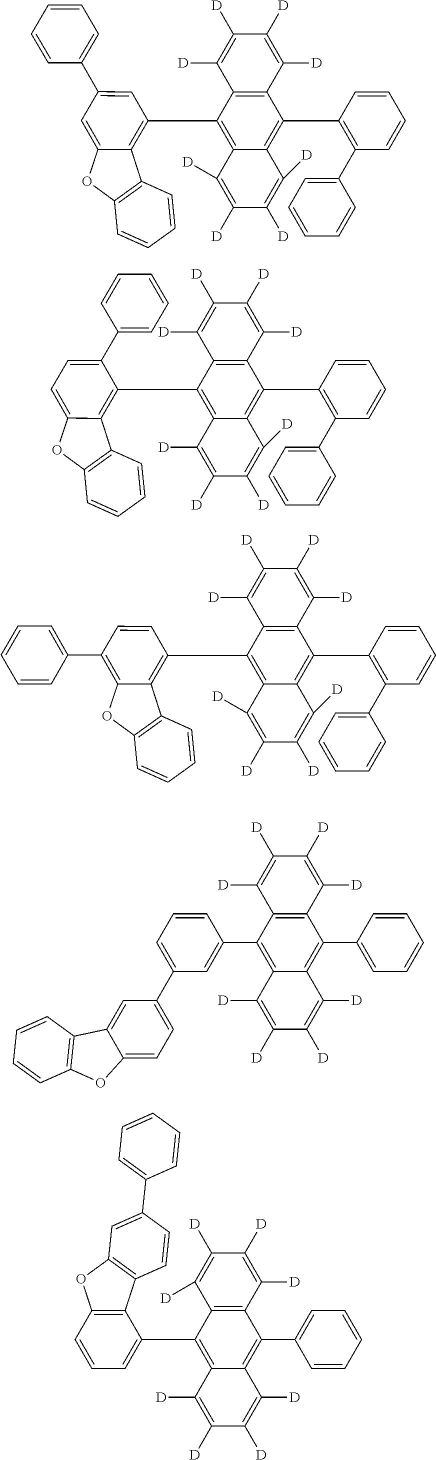

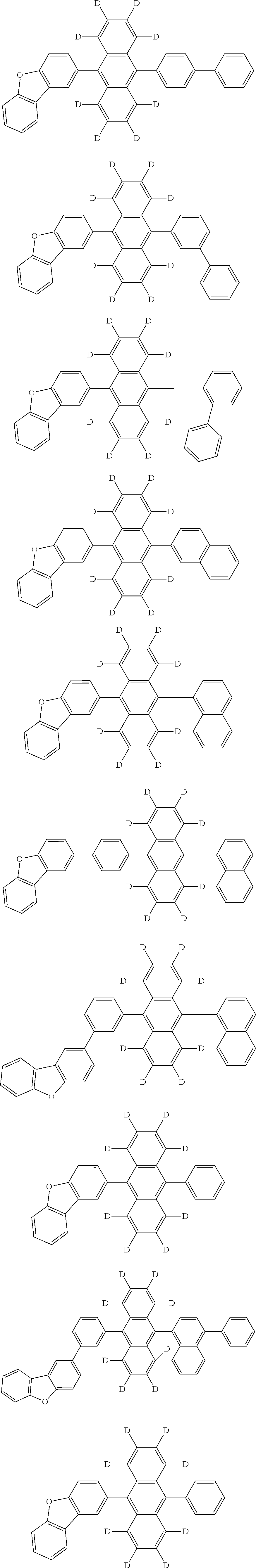

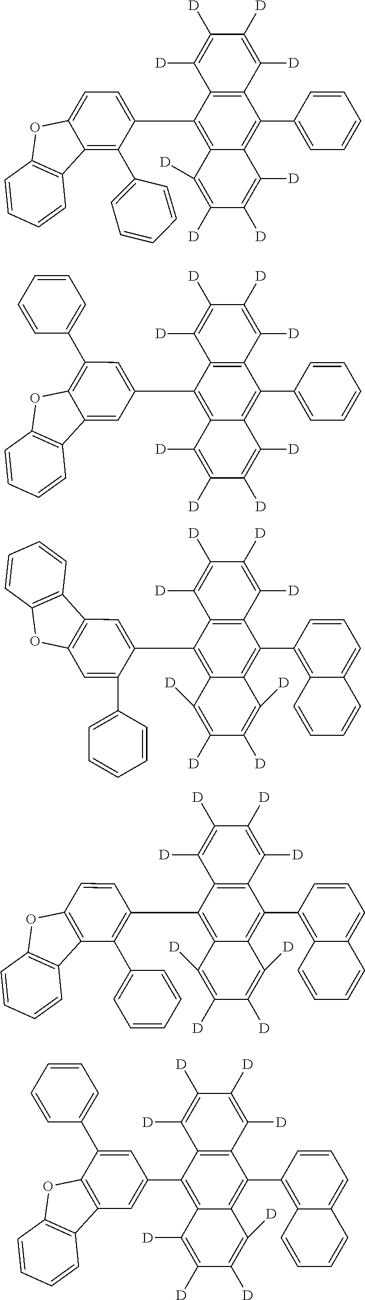

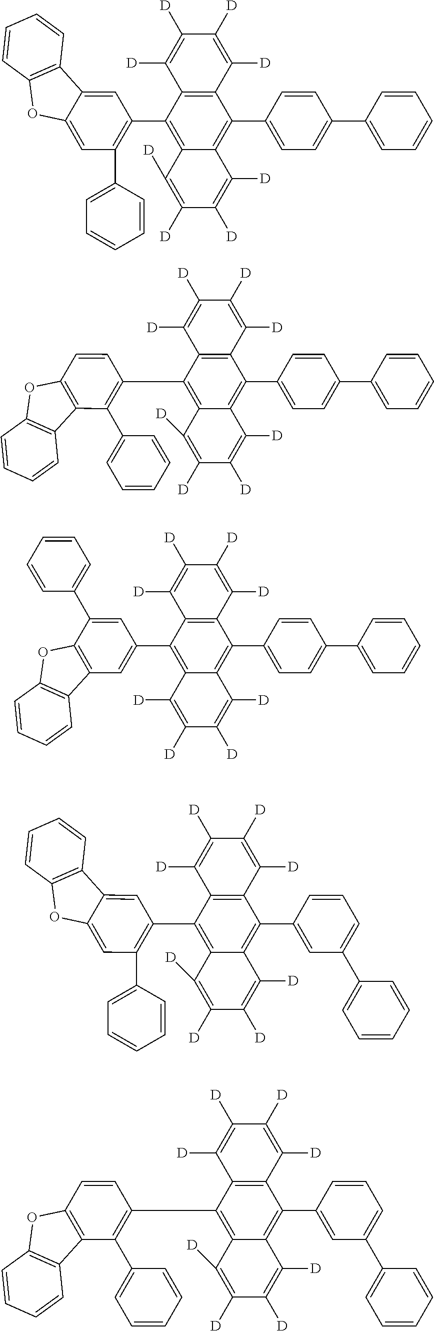

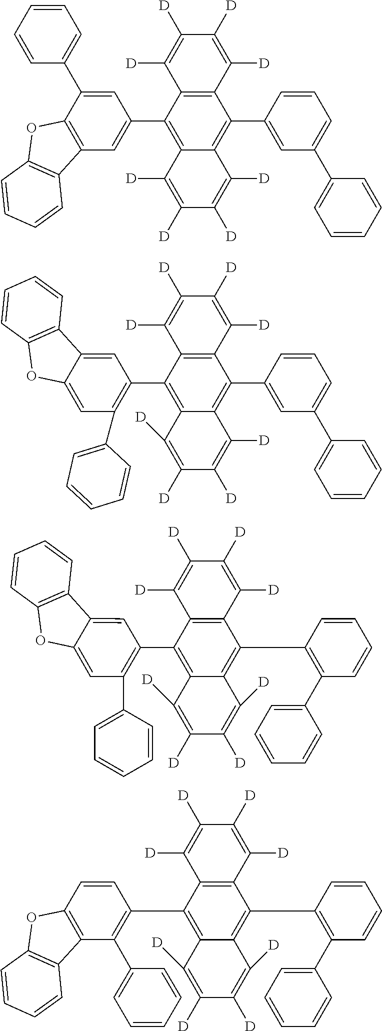

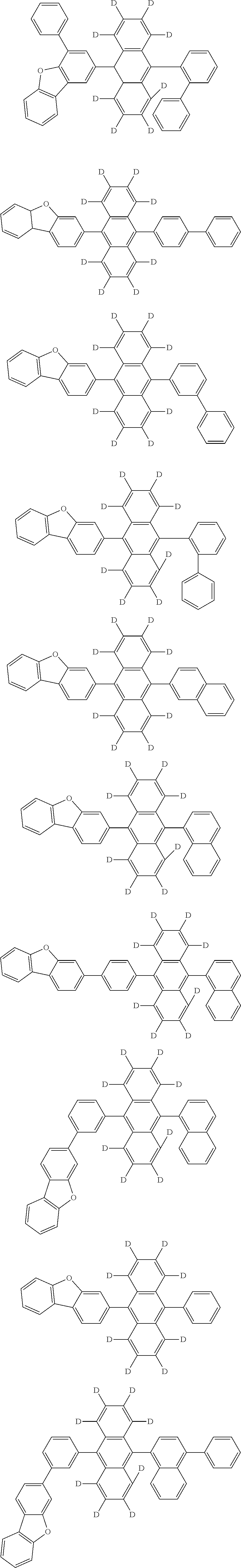

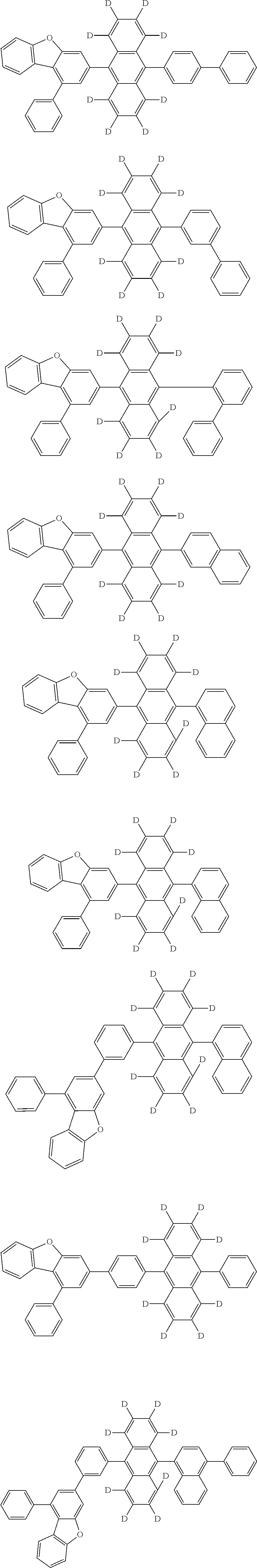

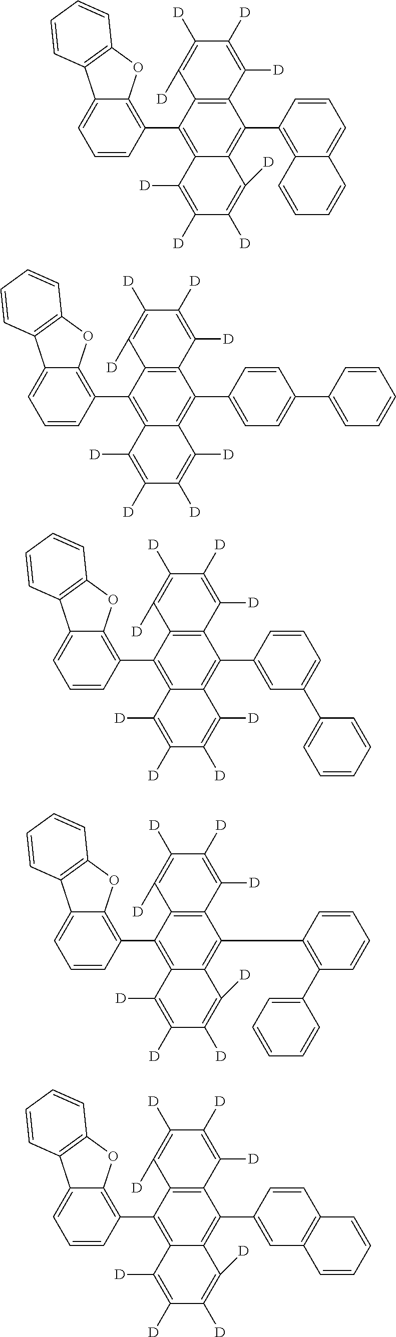

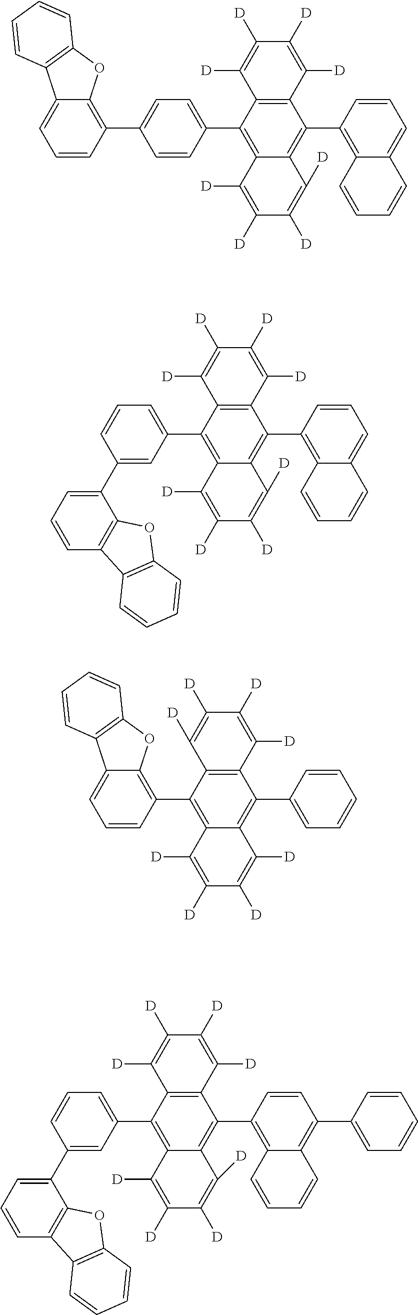 [SMILES: [2H]c1c([2H])c([2H])c2c(-c3cccc4ccccc34)c3c([2H])c([2H])c([2H])c([2H])c3c(-c3ccc(-c4cccc5c4oc4ccccc45)cc3)c2c1[2H] ; [2H]c1c([2H])c([2H])c2c(-c3cccc4ccccc34)c3c([2H])c([2H])c([2H])c([2H])c3c(-c3cccc(-c4cccc5c4oc4ccccc45)c3)c2c1[2H] ; [2H]c1c([2H])c([2H])c2c(-c3cccc4c3oc3ccccc34)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4cccc5c4oc4ccccc45)c3)c2c1[2H]]

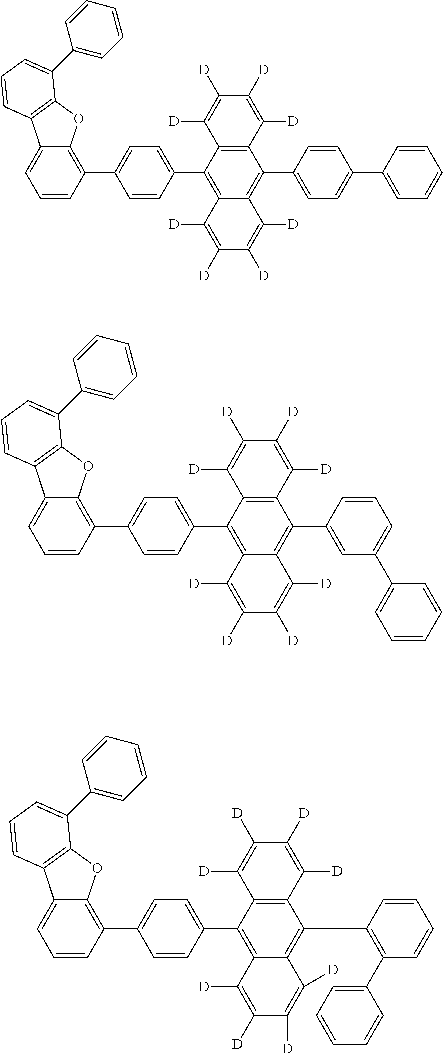 [SMILES: [2H]c1c([2H])c([2H])c2c(-c3ccc(-c4cccc5c4oc4c(-c6ccccc6)cccc45)cc3)c3c([2H])c([2H])c([2H])c([2H])c3c(-c3ccc(-c4ccccc4)cc3)c2c1[2H] ; [2H]c1c([2H])c([2H])c2c(-c3cccc(-c4ccccc4)c3)c3c([2H])c([2H])c([2H])c([2H])c3c(-c3ccc(-c4cccc5c4oc4c(-c6ccccc6)cccc45)cc3)c2c1[2H] ; [2H]c1c([2H])c([2H])c2c(-c3ccccc3-c3ccccc3)c3c([2H])c([2H])c([2H])c([2H])c3c(-c3ccc(-c4cccc5c4oc4c(-c6ccccc6)cccc45)cc3)c2c1[2H]]

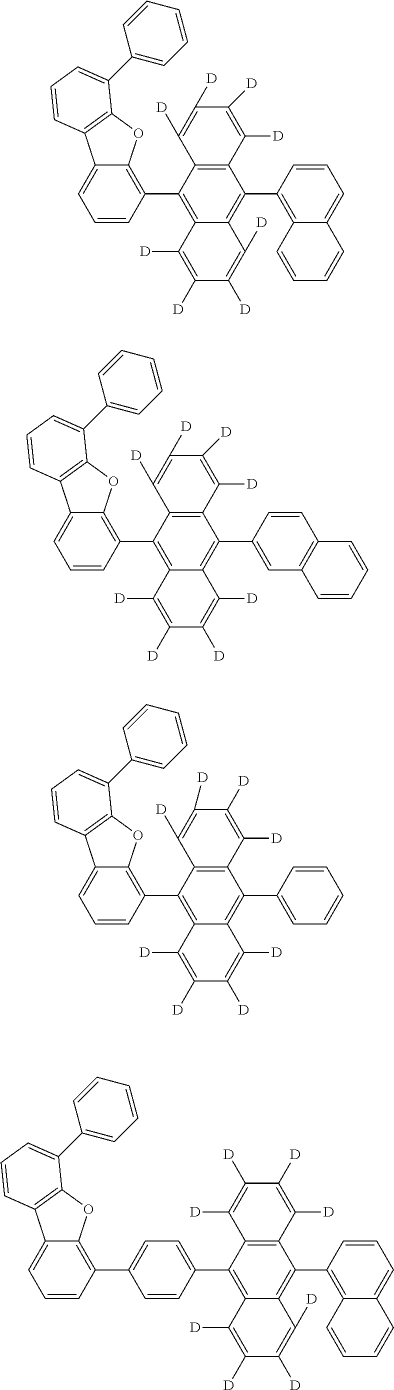 [SMILES: [2H]c1c([2H])c([2H])c2c(-c3cccc4c3oc3c(-c5ccccc5)cccc34)c3c([2H])c([2H])c([2H])c([2H])c3c(-c3cccc4ccccc34)c2c1[2H] ; [2H]c1c([2H])c([2H])c2c(-c3cccc4c3oc3c(-c5ccccc5)cccc34)c3c([2H])c([2H])c([2H])c([2H])c3c(-c3ccc4ccccc4c3)c2c1[2H] ; [2H]c1c([2H])c([2H])c2c(-c3cccc4c3oc3c(-c5ccccc5)cccc34)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc(-c4cccc5c4oc4c(-c6ccccc6)cccc45)cc3)c2c1[2H]]

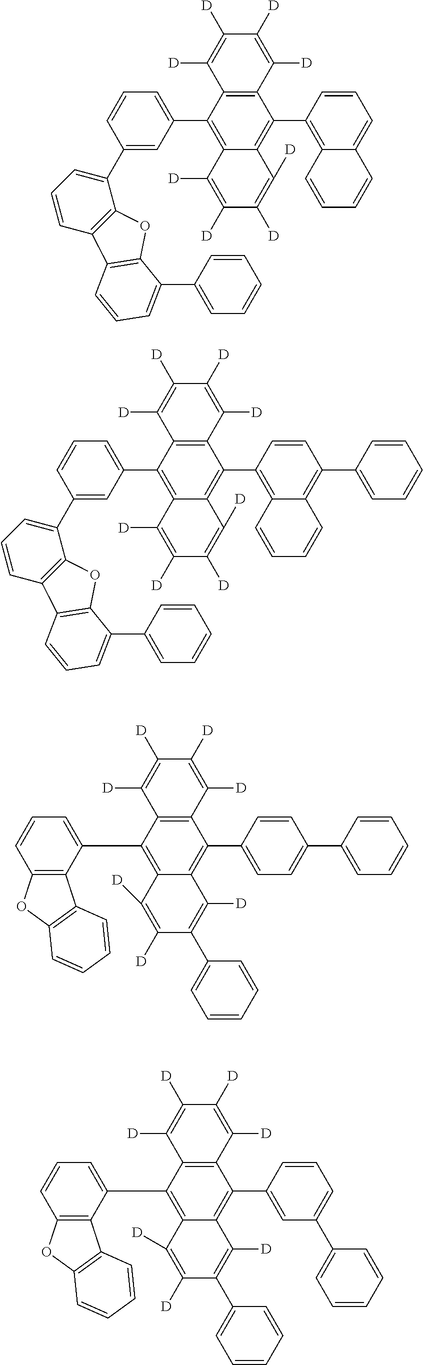

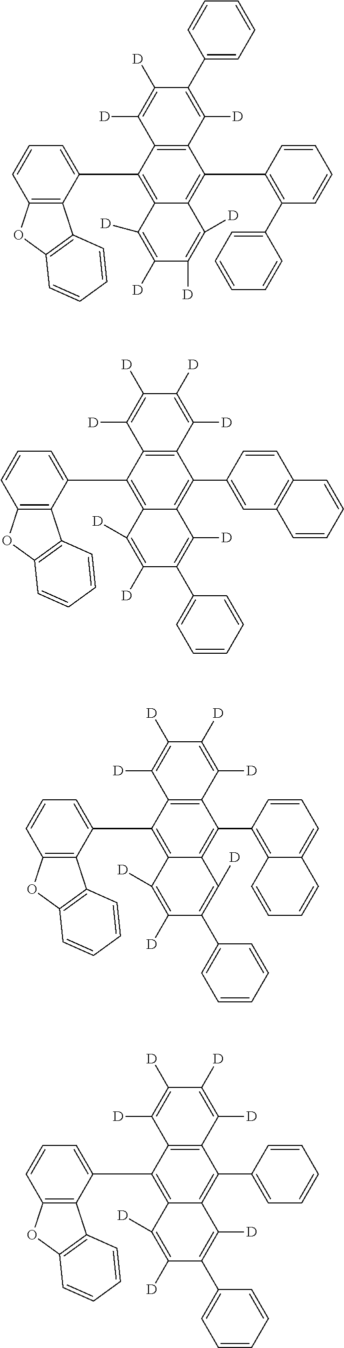 [SMILES: [2H]c1c([2H])c([2H])c2c(-c3cccc4oc5ccccc5c34)c3c([2H])c([2H])c(-c4ccccc4)c([2H])c3c(-c3ccccc3-c3ccccc3)c2c1[2H] ; [2H]c1c([2H])c([2H])c2c(-c3cccc4oc5ccccc5c34)c3c([2H])c([2H])c(-c4ccccc4)c([2H])c3c(-c3ccc4ccccc4c3)c2c1[2H] ; [2H]c1c([2H])c([2H])c2c(-c3cccc4oc5ccccc5c34)c3c([2H])c([2H])c(-c4ccccc4)c([2H])c3c(-c3cccc4ccccc34)c2c1[2H] ; [2H]c1c([2H])c([2H])c2c(-c3cccc4oc5ccccc5c34)c3c([2H])c([2H])c(-c4ccccc4)c([2H])c3c(-c3ccccc3)c2c1[2H]]

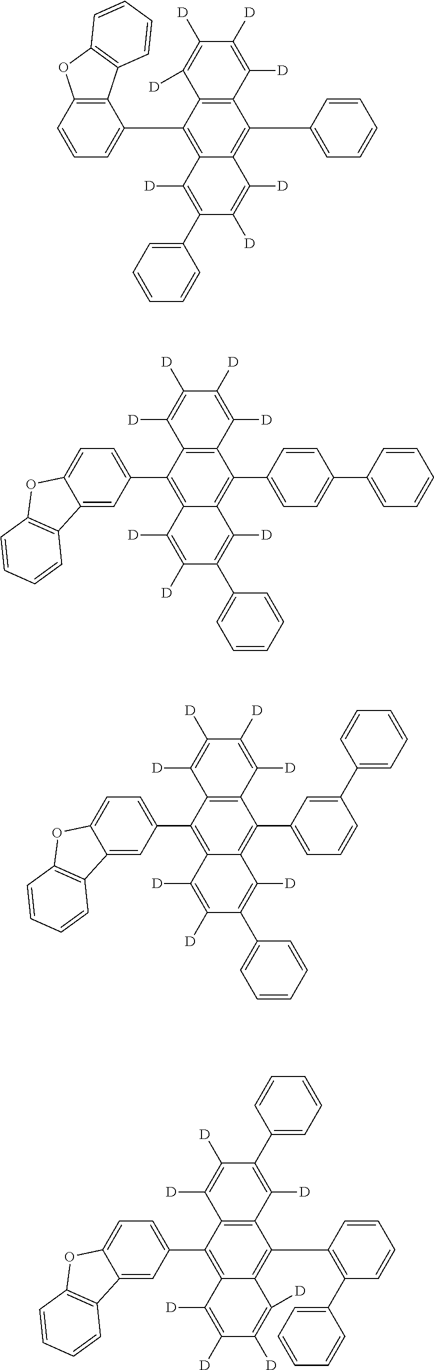 [SMILES: [2H]c1c([2H])c([2H])c2c(-c3cccc4oc5ccccc5c34)c3c([2H])c(-c4ccccc4)c([2H])c([2H])c3c(-c3ccccc3)c2c1[2H] ; [2H]c1c([2H])c([2H])c2c(-c3ccc(-c4ccccc4)cc3)c3c([2H])c(-c4ccccc4)c([2H])c([2H])c3c(-c3ccc4oc5ccccc5c4c3)c2c1[2H] ; [2H]c1c([2H])c([2H])c2c(-c3cccc(-c4ccccc4)c3)c3c([2H])c(-c4ccccc4)c([2H])c([2H])c3c(-c3ccc4oc5ccccc5c4c3)c2c1[2H] ; [2H]c1c([2H])c([2H])c2c(-c3ccccc3-c3ccccc3)c3c([2H])c(-c4ccccc4)c([2H])c([2H])c3c(-c3ccc4oc5ccccc5c4c3)c2c1[2H]]

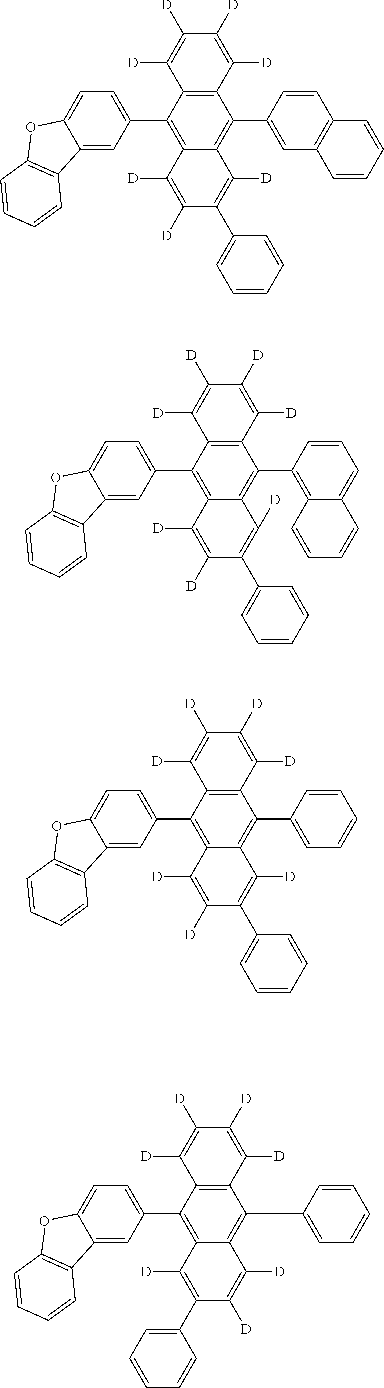 [SMILES: [2H]c1c([2H])c([2H])c2c(-c3ccc4ccccc4c3)c3c([2H])c(-c4ccccc4)c([2H])c([2H])c3c(-c3ccc4oc5ccccc5c4c3)c2c1[2H] ; [2H]c1c([2H])c([2H])c2c(-c3cccc4ccccc34)c3c([2H])c(-c4ccccc4)c([2H])c([2H])c3c(-c3ccc4oc5ccccc5c4c3)c2c1[2H] ; [2H]c1c([2H])c([2H])c2c(-c3ccccc3)c3c([2H])c(-c4ccccc4)c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c(-c4ccccc4)c([2H])c([2H])c3c(-c3ccccc3)c2c1[2H]]

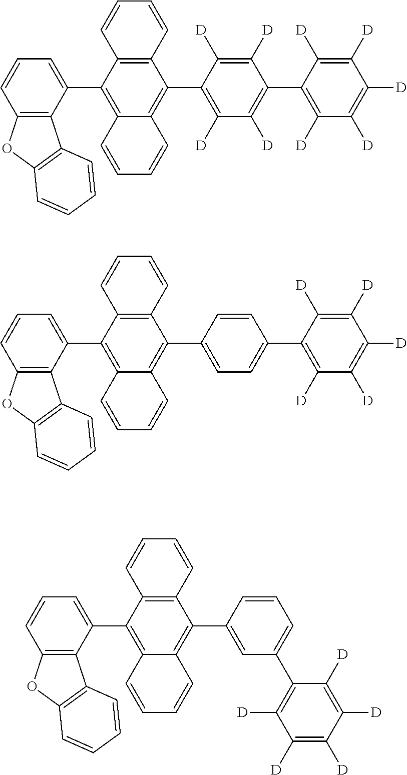 [SMILES: [2H]c1c([2H])c([2H])c(-c2c([2H])c([2H])c(-c3c4ccccc4c(-c4cccc5oc6ccccc6c45)c4ccccc34)c([2H])c2[2H])c([2H])c1[2H] ; [2H]c1c([2H])c([2H])c(-c2ccc(-c3c4ccccc4c(-c4cccc5oc6ccccc6c45)c4ccccc34)cc2)c([2H])c1[2H] ; [2H]c1c([2H])c([2H])c(-c2cccc(-c3c4ccccc4c(-c4cccc5oc6ccccc6c45)c4ccccc34)c2)c([2H])c1[2H]]

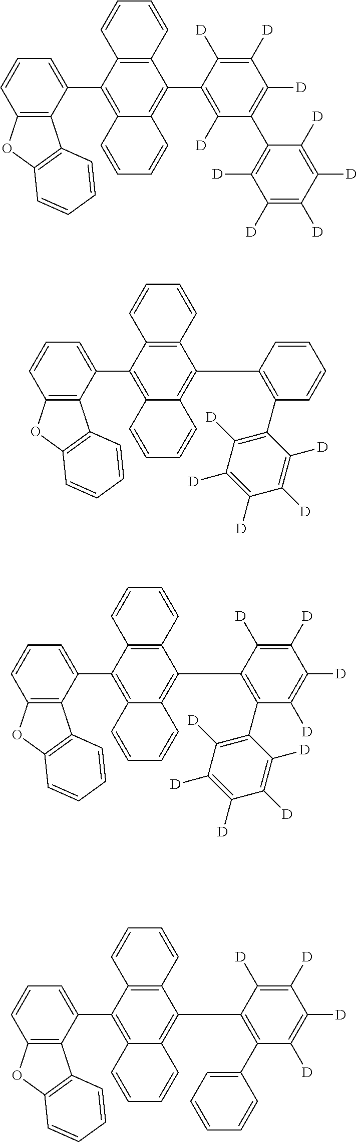 [SMILES: [2H]c1c([2H])c([2H])c(-c2c([2H])c([2H])c([2H])c(-c3c4ccccc4c(-c4cccc5oc6ccccc6c45)c4ccccc34)c2[2H])c([2H])c1[2H] ; [2H]c1c([2H])c([2H])c(-c2ccccc2-c2c3ccccc3c(-c3cccc4oc5ccccc5c34)c3ccccc23)c([2H])c1[2H] ; [2H]c1c([2H])c([2H])c(-c2c([2H])c([2H])c([2H])c([2H])c2-c2c3ccccc3c(-c3cccc4oc5ccccc5c34)c3ccccc23)c([2H])c1[2H] ; [2H]c1c([2H])c([2H])c(-c2c3ccccc3c(-c3cccc4oc5ccccc5c34)c3ccccc23)c(-c2ccccc2)c1[2H]]

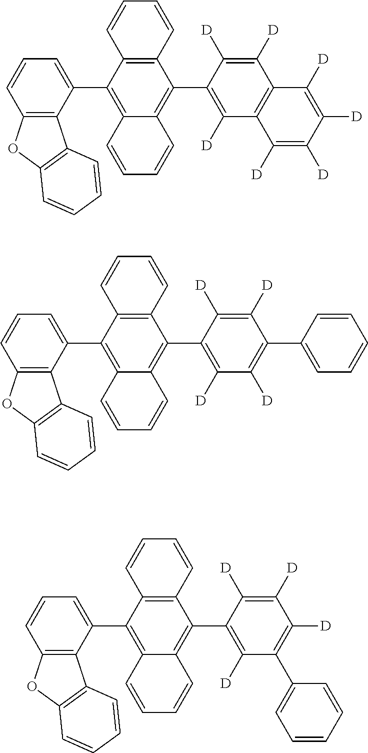 [SMILES: [2H]c1c([2H])c([2H])c2c([2H])c(-c3c4ccccc4c(-c4cccc5oc6ccccc6c45)c4ccccc34)c([2H])c([2H])c2c1[2H] ; [2H]c1c([2H])c(-c2c3ccccc3c(-c3cccc4oc5ccccc5c34)c3ccccc23)c([2H])c([2H])c1-c1ccccc1 ; [2H]c1c([2H])c(-c2ccccc2)c([2H])c(-c2c3ccccc3c(-c3cccc4oc5ccccc5c34)c3ccccc23)c1[2H]]

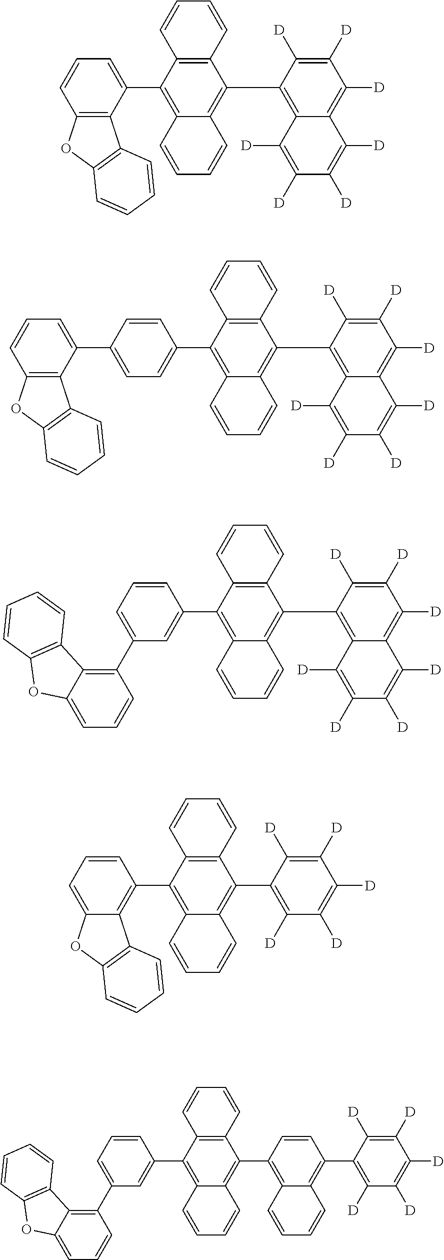

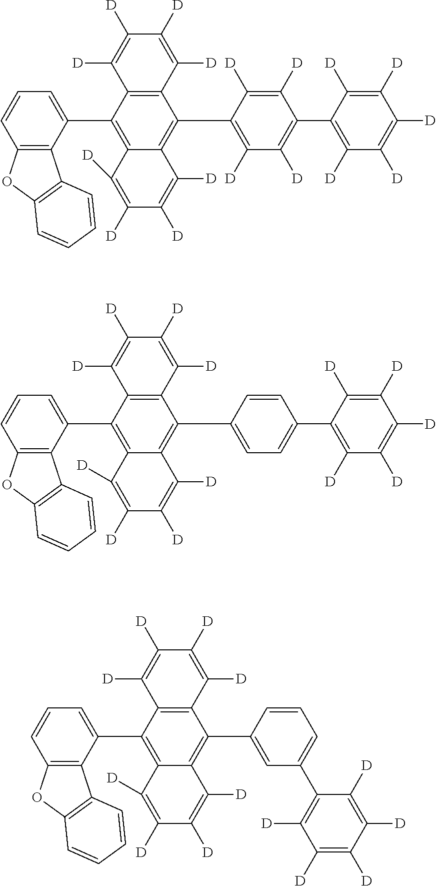 [SMILES: [2H]c1c([2H])c([2H])c(-c2c([2H])c([2H])c(-c3c4c([2H])c([2H])c([2H])c([2H])c4c(-c4cccc5oc6ccccc6c45)c4c([2H])c([2H])c([2H])c([2H])c34)c([2H])c2[2H])c([2H])c1[2H] ; [2H]c1c([2H])c([2H])c(-c2ccc(-c3c4c([2H])c([2H])c([2H])c([2H])c4c(-c4cccc5oc6ccccc6c45)c4c([2H])c([2H])c([2H])c([2H])c34)cc2)c([2H])c1[2H] ; [2H]c1c([2H])c([2H])c(-c2cccc(-c3c4c([2H])c([2H])c([2H])c([2H])c4c(-c4cccc5oc6ccccc6c45)c4c([2H])c([2H])c([2H])c([2H])c34)c2)c([2H])c1[2H]]

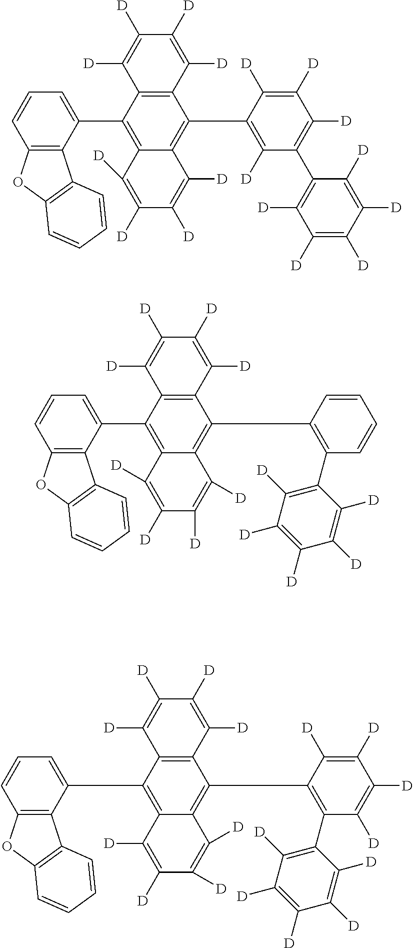 [SMILES: [2H]c1c([2H])c([2H])c(-c2c([2H])c([2H])c([2H])c(-c3c4c([2H])c([2H])c([2H])c([2H])c4c(-c4cccc5oc6ccccc6c45)c4c([2H])c([2H])c([2H])c([2H])c34)c2[2H])c([2H])c1[2H] ; [2H]c1c([2H])c([2H])c(-c2ccccc2-c2c3c([2H])c([2H])c([2H])c([2H])c3c(-c3cccc4oc5ccccc5c34)c3c([2H])c([2H])c([2H])c([2H])c23)c([2H])c1[2H] ; [2H]c1c([2H])c([2H])c(-c2c([2H])c([2H])c([2H])c([2H])c2-c2c3c([2H])c([2H])c([2H])c([2H])c3c(-c3cccc4oc5ccccc5c34)c3c([2H])c([2H])c([2H])c([2H])c23)c([2H])c1[2H]]

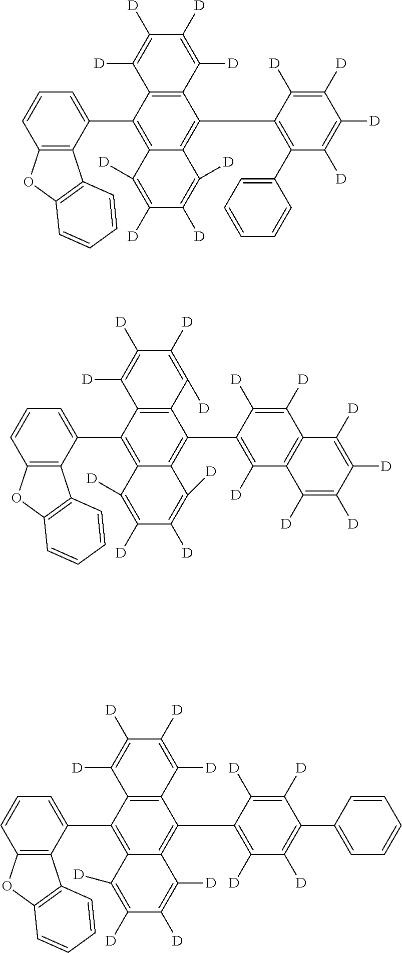 [SMILES: [2H]c1c([2H])c([2H])c(-c2c3c([2H])c([2H])c([2H])c([2H])c3c(-c3cccc4oc5ccccc5c34)c3c([2H])c([2H])c([2H])c([2H])c23)c(-c2ccccc2)c1[2H] ; [2H]c1c([2H])c([2H])c2c([2H])c(-c3c4c([2H])c([2H])c([2H])c([2H])c4c(-c4cccc5oc6ccccc6c45)c4c([2H])c([2H])c([2H])c([2H])c34)c([2H])c([2H])c2c1[2H] ; [2H]c1c([2H])c(-c2c3c([2H])c([2H])c([2H])c([2H])c3c(-c3cccc4oc5ccccc5c34)c3c([2H])c([2H])c([2H])c([2H])c23)c([2H])c([2H])c1-c1ccccc1]

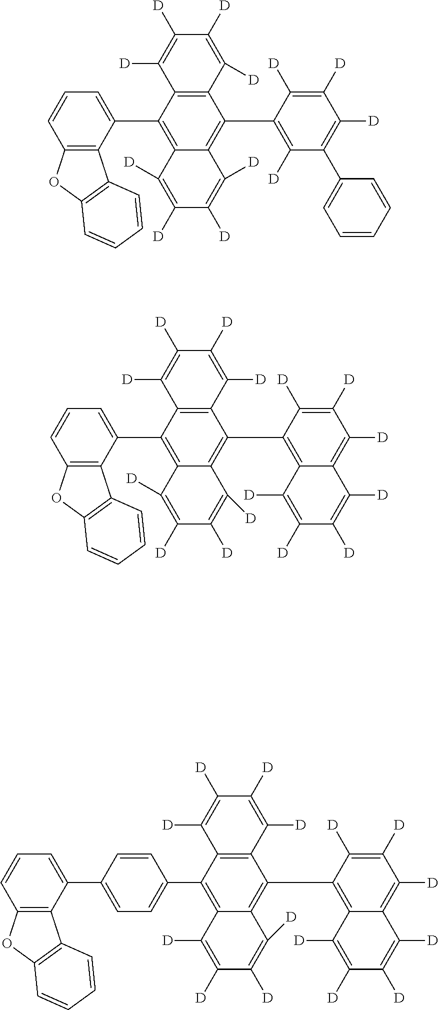 [SMILES: [2H]c1c([2H])c(-c2ccccc2)c([2H])c(-c2c3c([2H])c([2H])c([2H])c([2H])c3c(-c3cccc4oc5ccccc5c34)c3c([2H])c([2H])c([2H])c([2H])c23)c1[2H] ; [2H]c1c([2H])c([2H])c2c(-c3c4c([2H])c([2H])c([2H])c([2H])c4c(-c4cccc5oc6ccccc6c45)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4ccc(-c5cccc6oc7ccccc7c56)cc4)c4c([2H])c([2H])c([2H])c([2H])c34)c([2H])c([2H])c([2H])c2c1[2H]]

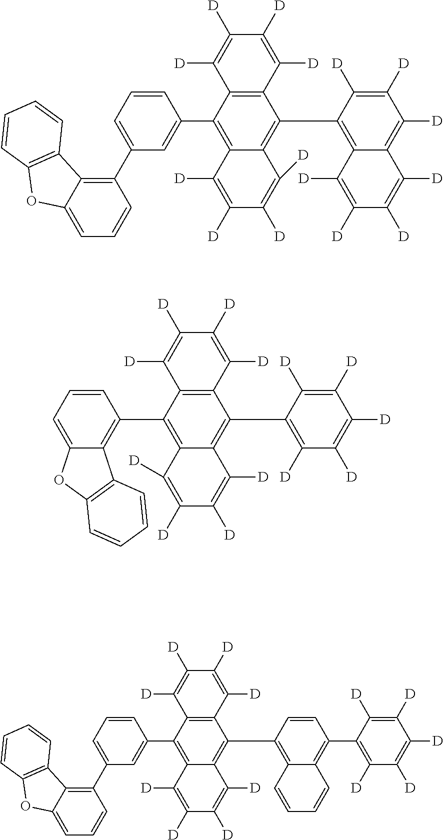 [SMILES: [2H]c1c([2H])c([2H])c2c(-c3c4c([2H])c([2H])c([2H])c([2H])c4c(-c4cccc(-c5cccc6oc7ccccc7c56)c4)c4c([2H])c([2H])c([2H])c([2H])c34)c([2H])c([2H])c([2H])c2c1[2H] ; [2H]c1c([2H])c([2H])c(-c2c3c([2H])c([2H])c([2H])c([2H])c3c(-c3cccc4oc5ccccc5c34)c3c([2H])c([2H])c([2H])c([2H])c23)c([2H])c1[2H] ; [2H]c1c([2H])c([2H])c(-c2ccc(-c3c4c([2H])c([2H])c([2H])c([2H])c4c(-c4cccc(-c5cccc6oc7ccccc7c56)c4)c4c([2H])c([2H])c([2H])c([2H])c34)c3ccccc23)c([2H])c1[2H]]

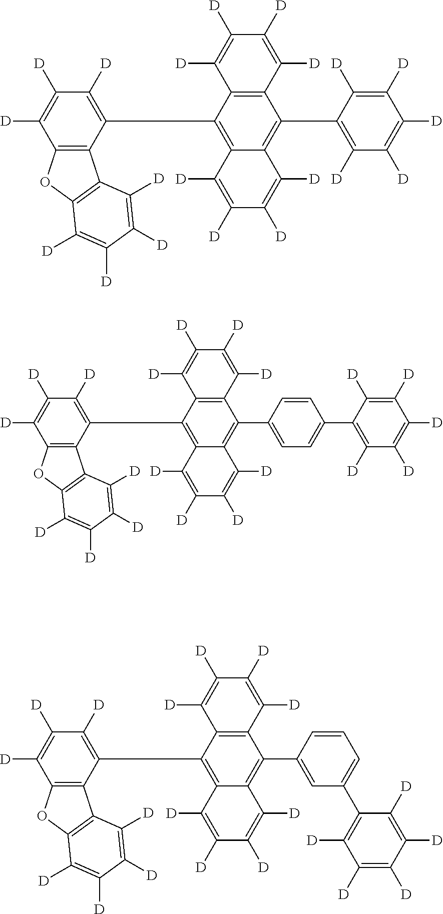

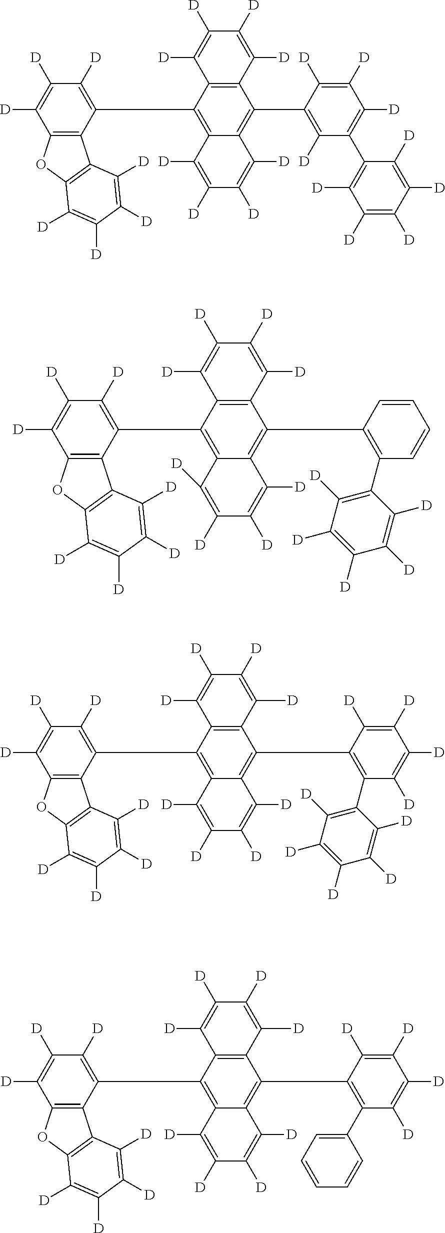

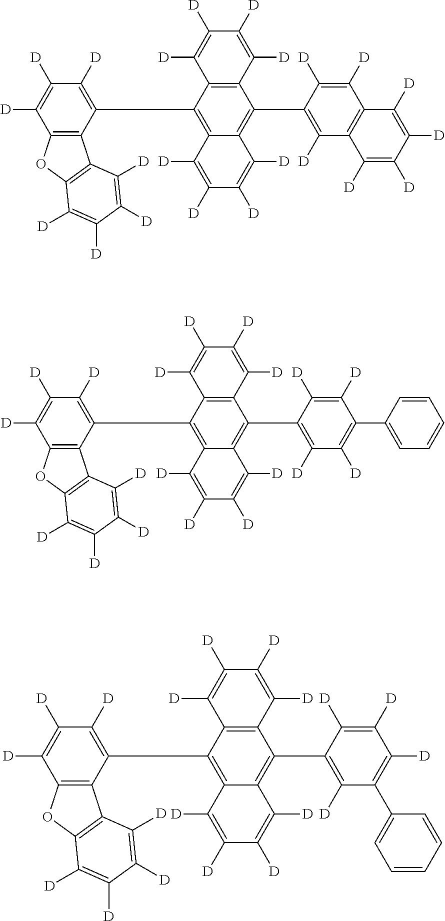

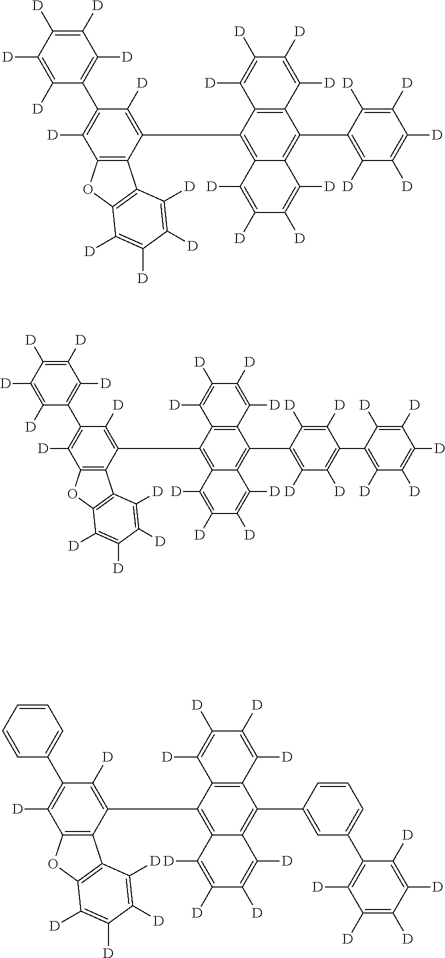

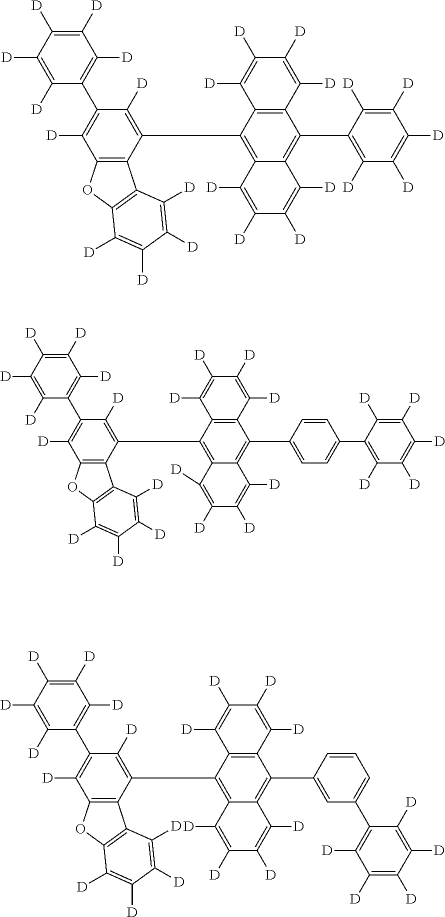

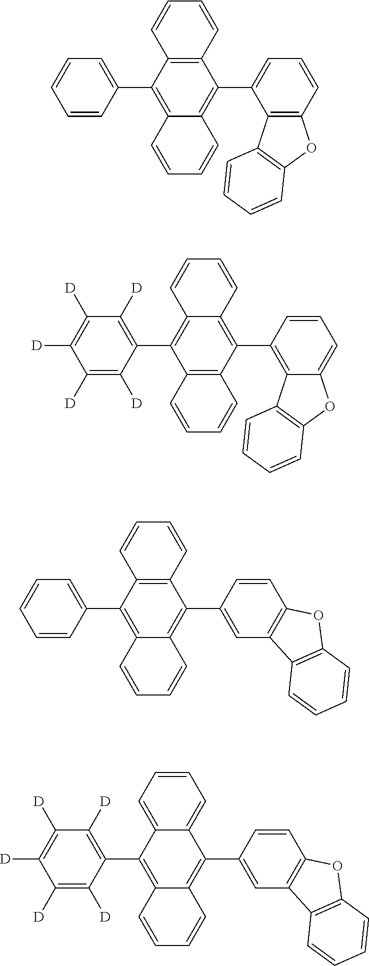 [SMILES: c1ccc(-c2c3ccccc3c(-c3cccc4oc5ccccc5c34)c3ccccc23)cc1 ; [2H]c1c([2H])c([2H])c(-c2c3ccccc3c(-c3cccc4oc5ccccc5c34)c3ccccc23)c([2H])c1[2H] ; c1ccc(-c2c3ccccc3c(-c3ccc4oc5ccccc5c4c3)c3ccccc23)cc1 ; [2H]c1c([2H])c([2H])c(-c2c3ccccc3c(-c3ccc4oc5ccccc5c4c3)c3ccccc23)c([2H])c1[2H]]

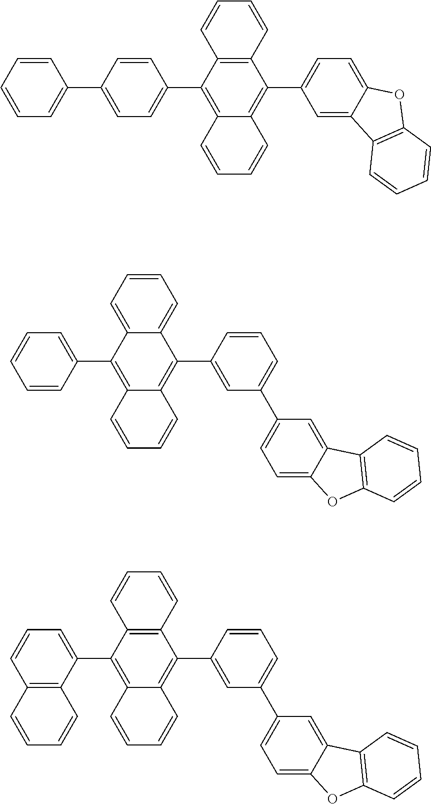 [SMILES: c1ccc(-c2ccc(-c3c4ccccc4c(-c4ccc5oc6ccccc6c5c4)c4ccccc34)cc2)cc1 ; c1ccc(-c2c3ccccc3c(-c3cccc(-c4ccc5oc6ccccc6c5c4)c3)c3ccccc23)cc1 ; c1cc(-c2ccc3oc4ccccc4c3c2)cc(-c2c3ccccc3c(-c3cccc4ccccc34)c3ccccc23)c1]

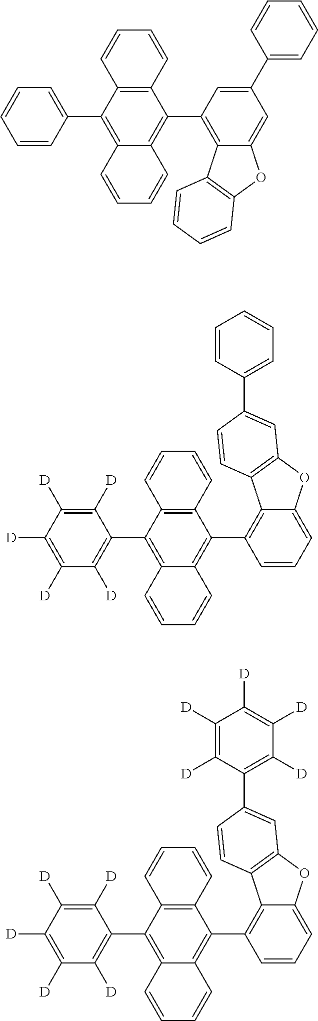 [SMILES: c1ccc(-c2cc(-c3c4ccccc4c(-c4ccccc4)c4ccccc34)c3c(c2)oc2ccccc23)cc1 ; [2H]c1c([2H])c([2H])c(-c2c3ccccc3c(-c3cccc4oc5cc(-c6ccccc6)ccc5c34)c3ccccc23)c([2H])c1[2H] ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H]]

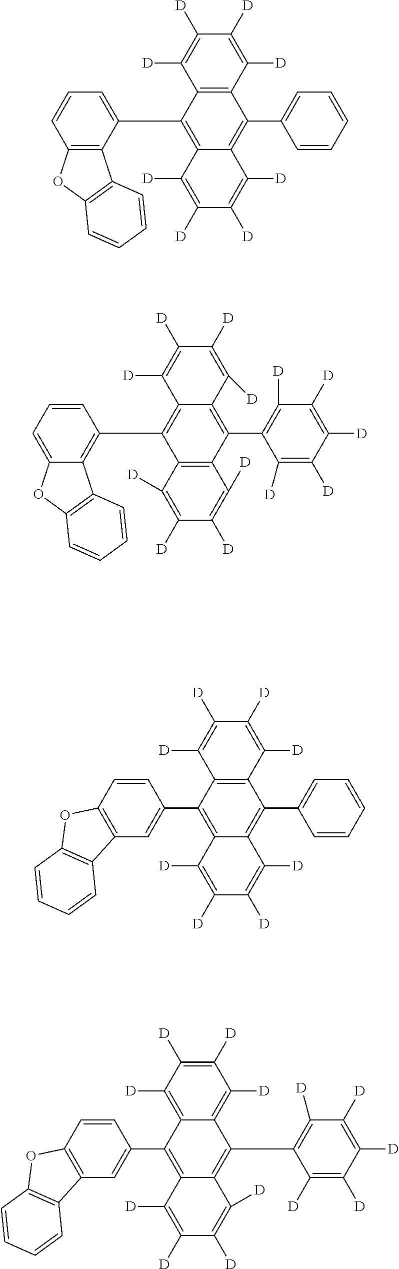 [SMILES: [2H]c1c([2H])c([2H])c2c(-c3cccc4oc5ccccc5c34)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3cccc4oc5ccccc5c34)c3c([2H])c([2H])c([2H])c([2H])c23)c([2H])c1[2H] ; [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3ccc4oc5ccccc5c4c3)c3c([2H])c([2H])c([2H])c([2H])c23)c([2H])c1[2H]]

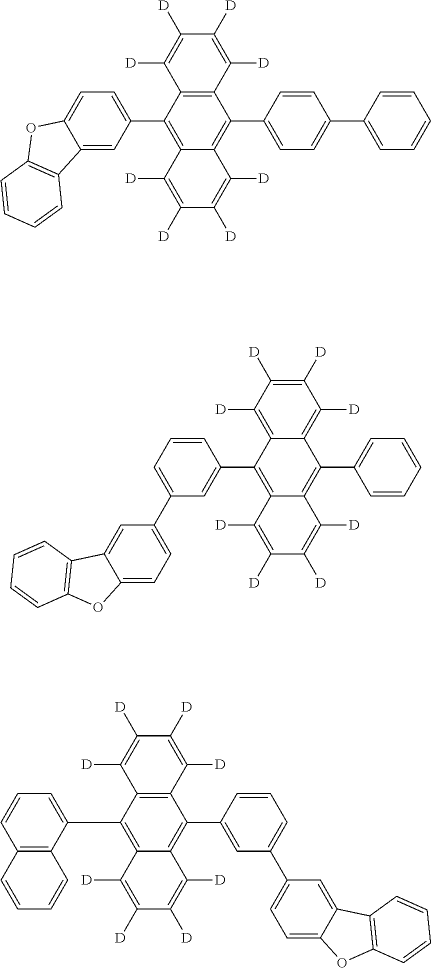 [SMILES: [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3cccc(-c4ccc5oc6ccccc6c5c4)c3)c2c1[2H]]

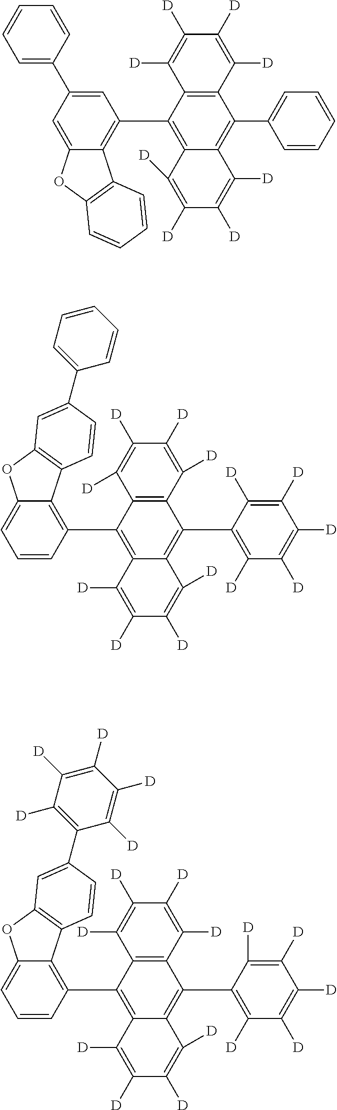 [SMILES: [2H]c1c([2H])c([2H])c2c(-c3cc(-c4ccccc4)cc4oc5ccccc5c34)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3cccc4oc5cc(-c6ccccc6)ccc5c34)c3c([2H])c([2H])c([2H])c([2H])c23)c([2H])c1[2H] ; [2H]c1c([2H])c([2H])c(-c2ccc3c(c2)oc2cccc(-c4c5c([2H])c([2H])c([2H])c([2H])c5c(-c5c([2H])c([2H])c([2H])c([2H])c5[2H])c5c([2H])c([2H])c([2H])c([2H])c45)c23)c([2H])c1[2H]]

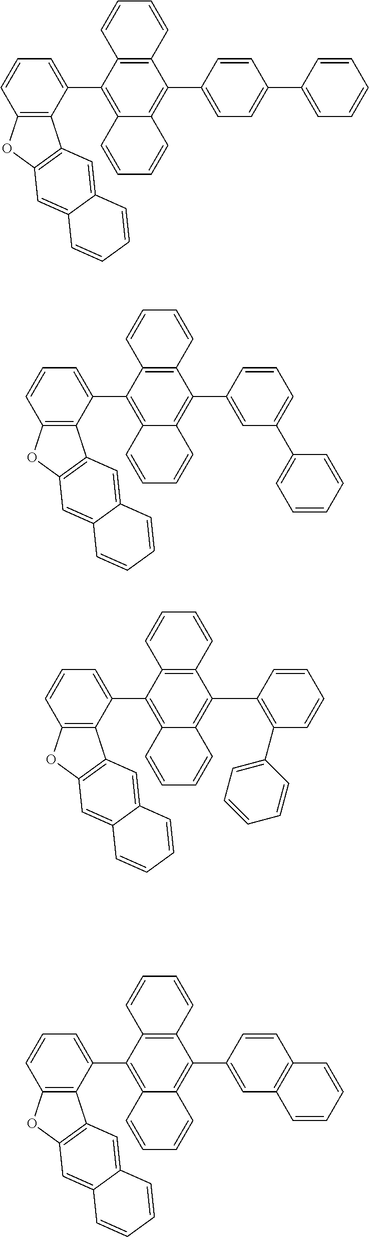 [SMILES: c1ccc(-c2ccc(-c3c4ccccc4c(-c4cccc5oc6cc7ccccc7cc6c45)c4ccccc34)cc2)cc1 ; c1ccc(-c2cccc(-c3c4ccccc4c(-c4cccc5oc6cc7ccccc7cc6c45)c4ccccc34)c2)cc1 ; c1ccc(-c2ccccc2-c2c3ccccc3c(-c3cccc4oc5cc6ccccc6cc5c34)c3ccccc23)cc1 ; c1ccc2cc(-c3c4ccccc4c(-c4cccc5oc6cc7ccccc7cc6c45)c4ccccc34)ccc2c1]

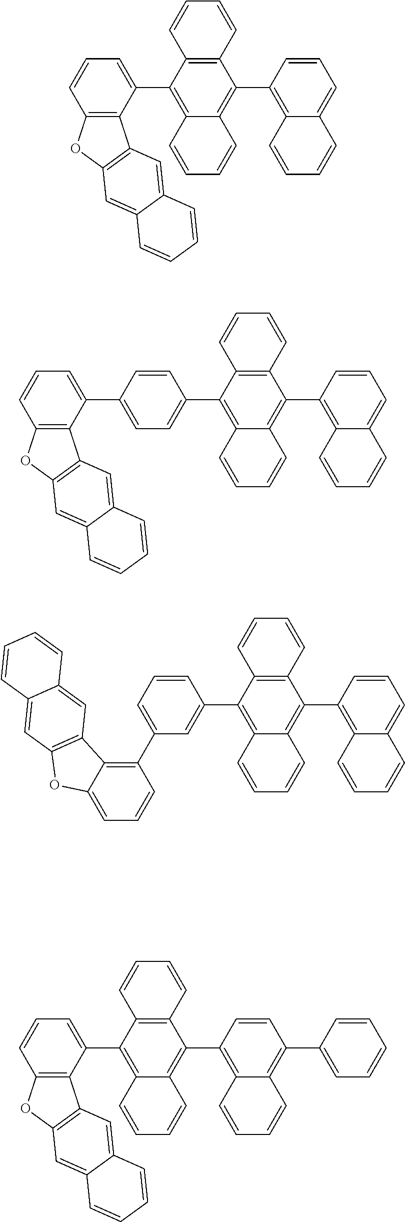 [SMILES: c1ccc2cc3c(cc2c1)oc1cccc(-c2c4ccccc4c(-c4cccc5ccccc45)c4ccccc24)c13 ; c1ccc2cc3c(cc2c1)oc1cccc(-c2ccc(-c4c5ccccc5c(-c5cccc6ccccc56)c5ccccc45)cc2)c13 ; c1cc(-c2c3ccccc3c(-c3cccc4ccccc34)c3ccccc23)cc(-c2cccc3oc4cc5ccccc5cc4c23)c1 ; c1ccc(-c2ccc(-c3c4ccccc4c(-c4cccc5oc6cc7ccccc7cc6c45)c4ccccc34)c3ccccc23)cc1]

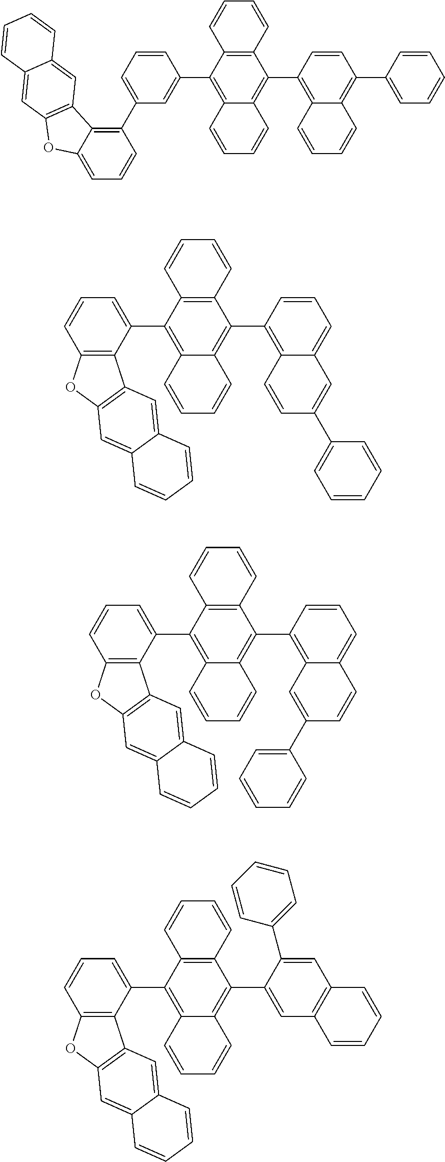 [SMILES: c1ccc(-c2ccc(-c3c4ccccc4c(-c4cccc(-c5cccc6oc7cc8ccccc8cc7c56)c4)c4ccccc34)c3ccccc23)cc1 ; c1ccc(-c2ccc3c(-c4c5ccccc5c(-c5cccc6oc7cc8ccccc8cc7c56)c5ccccc45)cccc3c2)cc1 ; c1ccc(-c2ccc3cccc(-c4c5ccccc5c(-c5cccc6oc7cc8ccccc8cc7c56)c5ccccc45)c3c2)cc1 ; c1ccc(-c2cc3ccccc3cc2-c2c3ccccc3c(-c3cccc4oc5cc6ccccc6cc5c34)c3ccccc23)cc1]

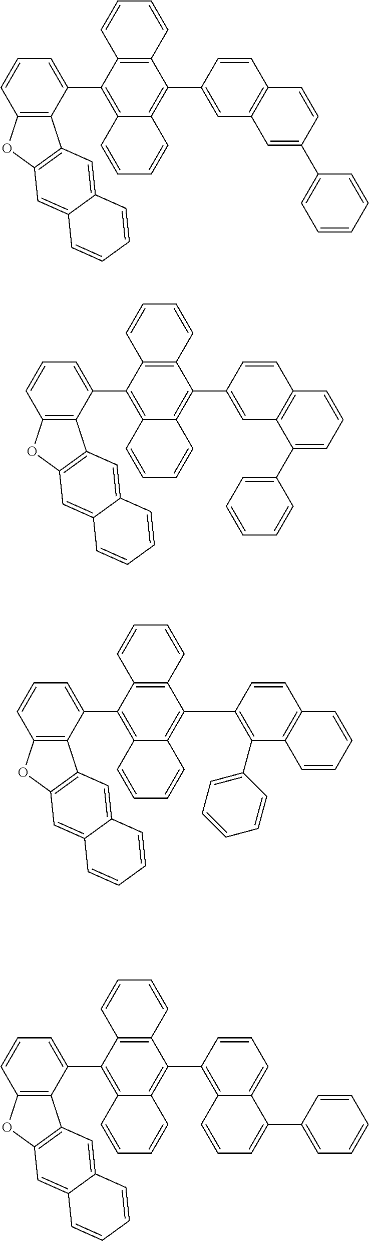 [SMILES: c1ccc(-c2ccc3ccc(-c4c5ccccc5c(-c5cccc6oc7cc8ccccc8cc7c56)c5ccccc45)cc3c2)cc1 ; c1ccc(-c2cccc3ccc(-c4c5ccccc5c(-c5cccc6oc7cc8ccccc8cc7c56)c5ccccc45)cc23)cc1 ; c1ccc(-c2c(-c3c4ccccc4c(-c4cccc5oc6cc7ccccc7cc6c45)c4ccccc34)ccc3ccccc23)cc1 ; c1ccc(-c2cccc3c(-c4c5ccccc5c(-c5cccc6oc7cc8ccccc8cc7c56)c5ccccc45)cccc23)cc1]

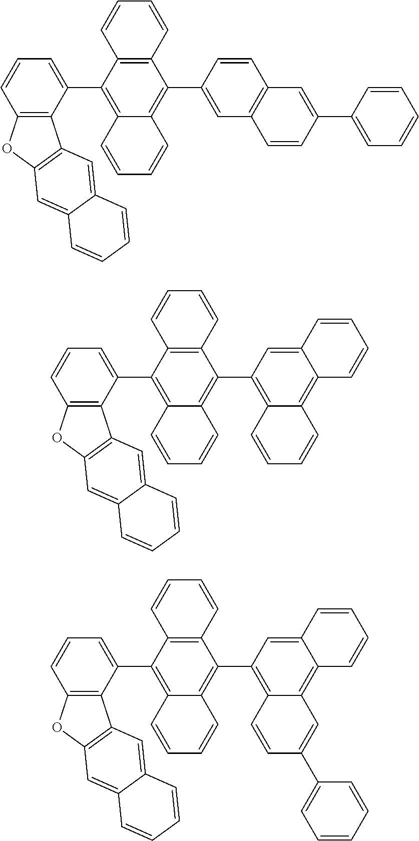

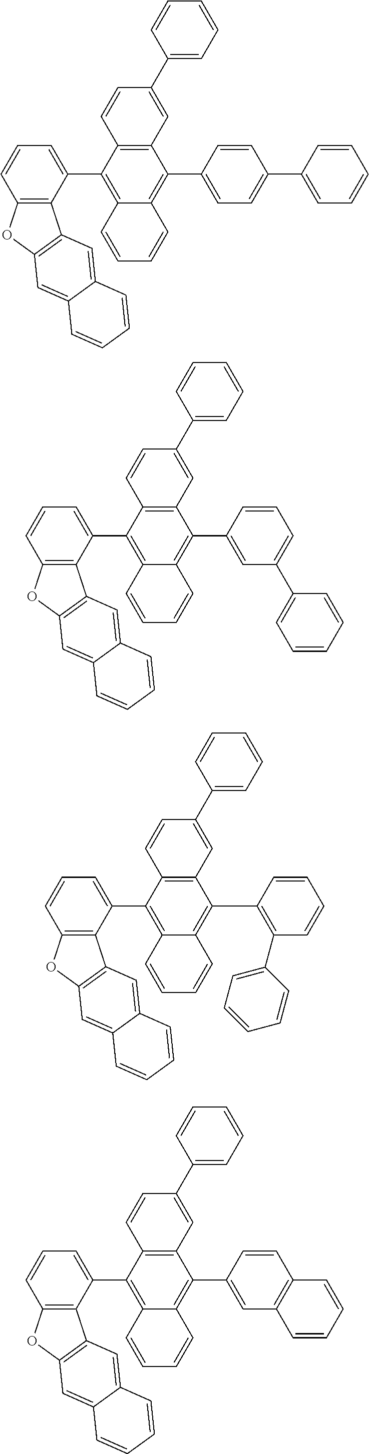 [SMILES: c1ccc(-c2ccc(-c3c4ccccc4c(-c4cccc5oc6cc7ccccc7cc6c45)c4ccc(-c5ccccc5)cc34)cc2)cc1 ; c1ccc(-c2cccc(-c3c4ccccc4c(-c4cccc5oc6cc7ccccc7cc6c45)c4ccc(-c5ccccc5)cc34)c2)cc1 ; c1ccc(-c2ccc3c(-c4cccc5oc6cc7ccccc7cc6c45)c4ccccc4c(-c4ccccc4-c4ccccc4)c3c2)cc1 ; c1ccc(-c2ccc3c(-c4cccc5oc6cc7ccccc7cc6c45)c4ccccc4c(-c4ccc5ccccc5c4)c3c2)cc1]

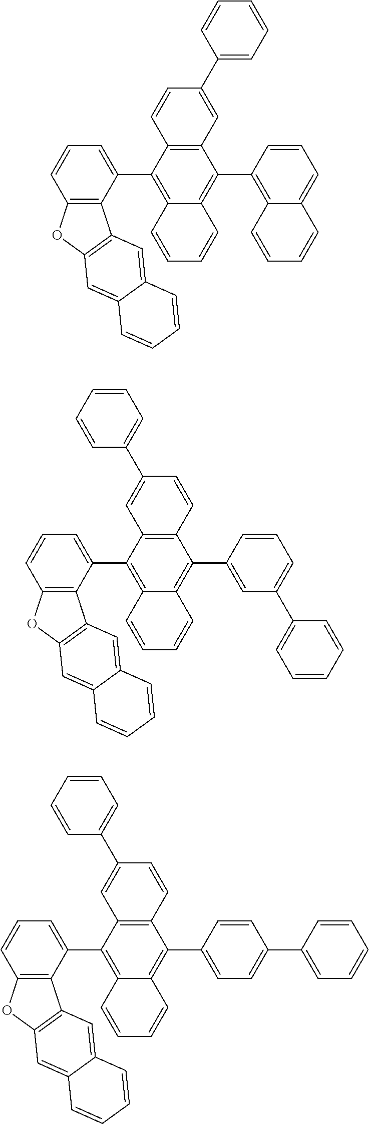 [SMILES: c1ccc(-c2ccc3c(-c4cccc5oc6cc7ccccc7cc6c45)c4ccccc4c(-c4cccc5ccccc45)c3c2)cc1 ; c1ccc(-c2cccc(-c3c4ccccc4c(-c4cccc5oc6cc7ccccc7cc6c45)c4cc(-c5ccccc5)ccc34)c2)cc1 ; c1ccc(-c2ccc(-c3c4ccccc4c(-c4cccc5oc6cc7ccccc7cc6c45)c4cc(-c5ccccc5)ccc34)cc2)cc1]

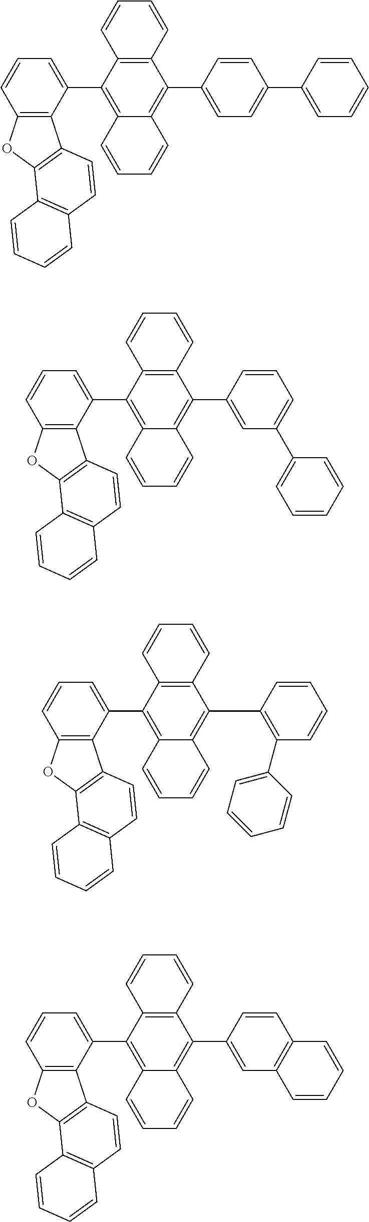 [SMILES: c1ccc(-c2ccc(-c3c4ccccc4c(-c4cccc5oc6c7ccccc7ccc6c45)c4ccccc34)cc2)cc1 ; c1ccc(-c2cccc(-c3c4ccccc4c(-c4cccc5oc6c7ccccc7ccc6c45)c4ccccc34)c2)cc1 ; c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1]

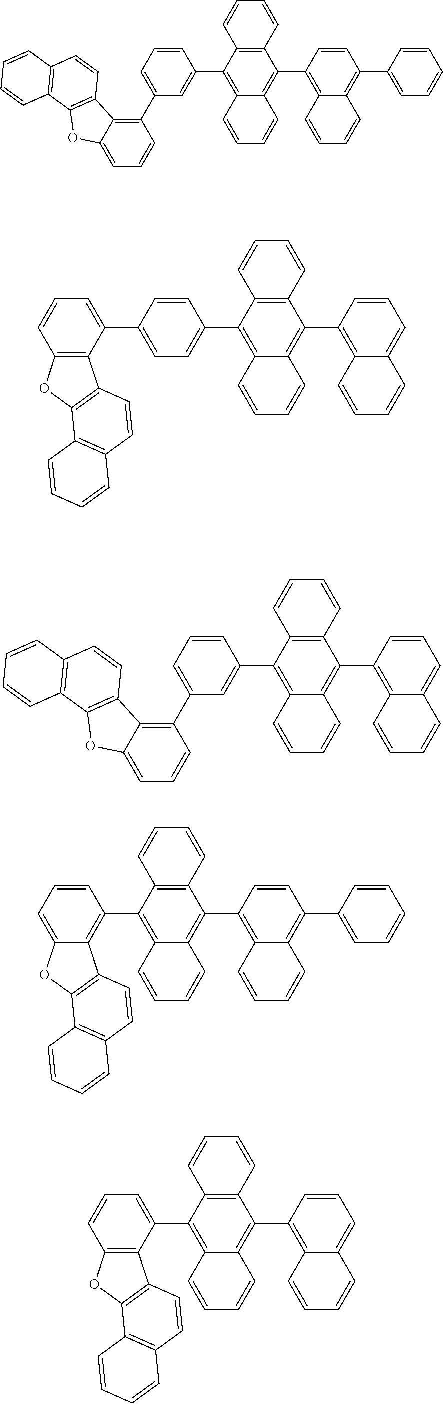 [SMILES: c1ccc(-c2ccc(-c3c4ccccc4c(-c4cccc(-c5cccc6oc7c8ccccc8ccc7c56)c4)c4ccccc34)c3ccccc23)cc1 ; c1ccc2c(-c3c4ccccc4c(-c4ccc(-c5cccc6oc7c8ccccc8ccc7c56)cc4)c4ccccc34)cccc2c1 ; c1cc(-c2c3ccccc3c(-c3cccc4ccccc34)c3ccccc23)cc(-c2cccc3oc4c5ccccc5ccc4c23)c1 ; c1ccc(-c2ccc(-c3c4ccccc4c(-c4cccc5oc6c7ccccc7ccc6c45)c4ccccc34)c3ccccc23)cc1 ; c1ccc2c(-c3c4ccccc4c(-c4cccc5oc6c7ccccc7ccc6c45)c4ccccc34)cccc2c1]

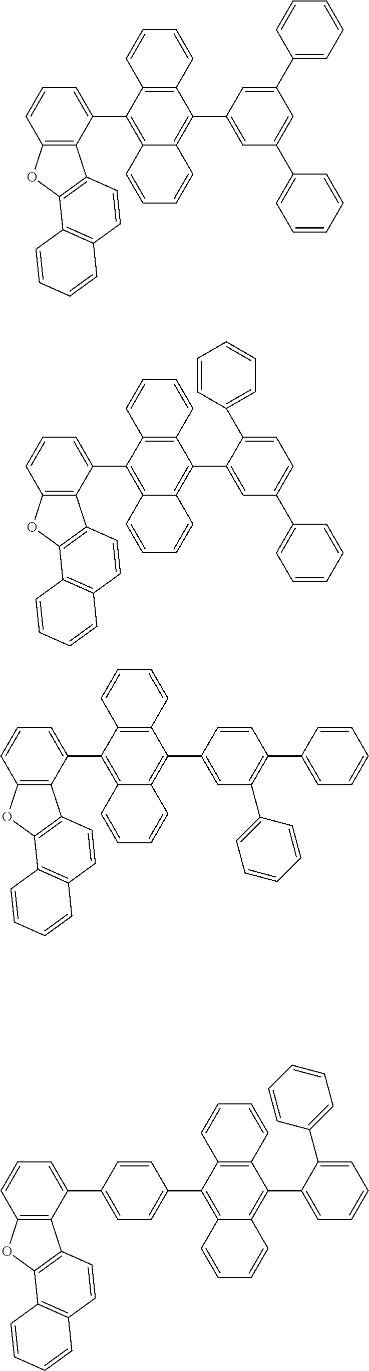 [SMILES: c1ccc(-c2cc(-c3ccccc3)cc(-c3c4ccccc4c(-c4cccc5oc6c7ccccc7ccc6c45)c4ccccc34)c2)cc1 ; c1ccc(-c2ccc(-c3ccccc3)c(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2-c2ccccc2)cc1 ; c1ccc(-c2ccccc2-c2c3ccccc3c(-c3ccc(-c4cccc5oc6c7ccccc7ccc6c45)cc3)c3ccccc23)cc1]

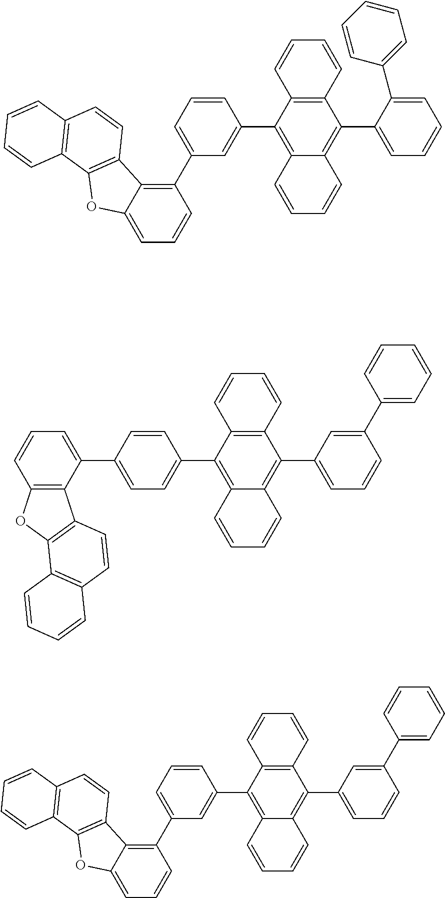 [SMILES: c1ccc(-c2ccccc2-c2c3ccccc3c(-c3cccc(-c4cccc5oc6c7ccccc7ccc6c45)c3)c3ccccc23)cc1 ; c1ccc(-c2cccc(-c3c4ccccc4c(-c4ccc(-c5cccc6oc7c8ccccc8ccc7c56)cc4)c4ccccc34)c2)cc1 ; c1ccc(-c2cccc(-c3c4ccccc4c(-c4cccc(-c5cccc6oc7c8ccccc8ccc7c56)c4)c4ccccc34)c2)cc1]

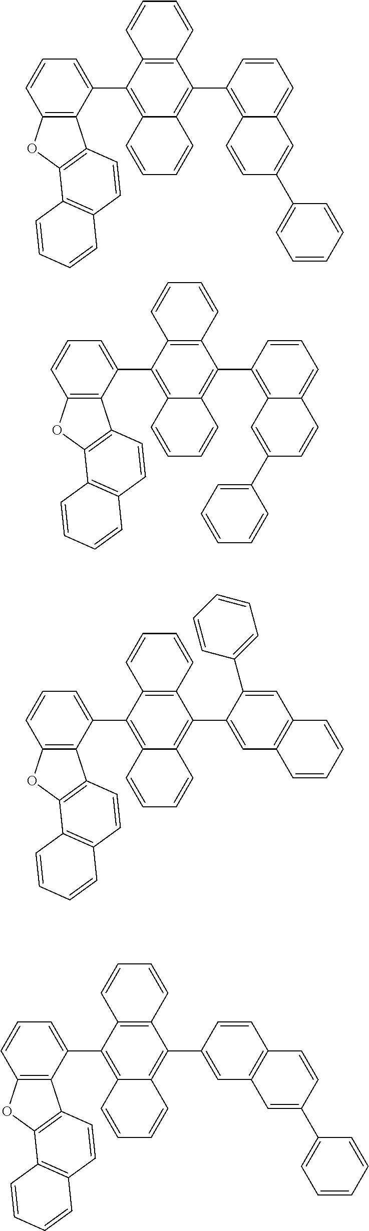 [SMILES: c1ccc(-c2ccc3c(-c4c5ccccc5c(-c5cccc6oc7c8ccccc8ccc7c56)c5ccccc45)cccc3c2)cc1 ; c1ccc(-c2ccc3cccc(-c4c5ccccc5c(-c5cccc6oc7c8ccccc8ccc7c56)c5ccccc45)c3c2)cc1 ; c1ccc(-c2cc3ccccc3cc2-c2c3ccccc3c(-c3cccc4oc5c6ccccc6ccc5c34)c3ccccc23)cc1 ; c1ccc(-c2ccc3ccc(-c4c5ccccc5c(-c5cccc6oc7c8ccccc8ccc7c56)c5ccccc45)cc3c2)cc1]

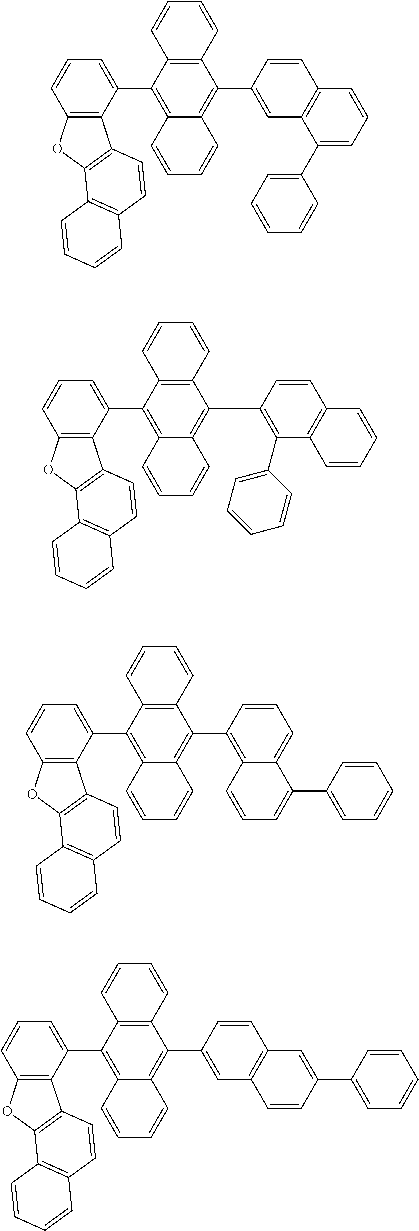 [SMILES: c1ccc(-c2cccc3ccc(-c4c5ccccc5c(-c5cccc6oc7c8ccccc8ccc7c56)c5ccccc45)cc23)cc1 ; c1ccc(-c2c(-c3c4ccccc4c(-c4cccc5oc6c7ccccc7ccc6c45)c4ccccc34)ccc3ccccc23)cc1 ; c1ccc(-c2cccc3c(-c4c5ccccc5c(-c5cccc6oc7c8ccccc8ccc7c56)c5ccccc45)cccc23)cc1 ; c1ccc(-c2ccc3cc(-c4c5ccccc5c(-c5cccc6oc7c8ccccc8ccc7c56)c5ccccc45)ccc3c2)cc1]

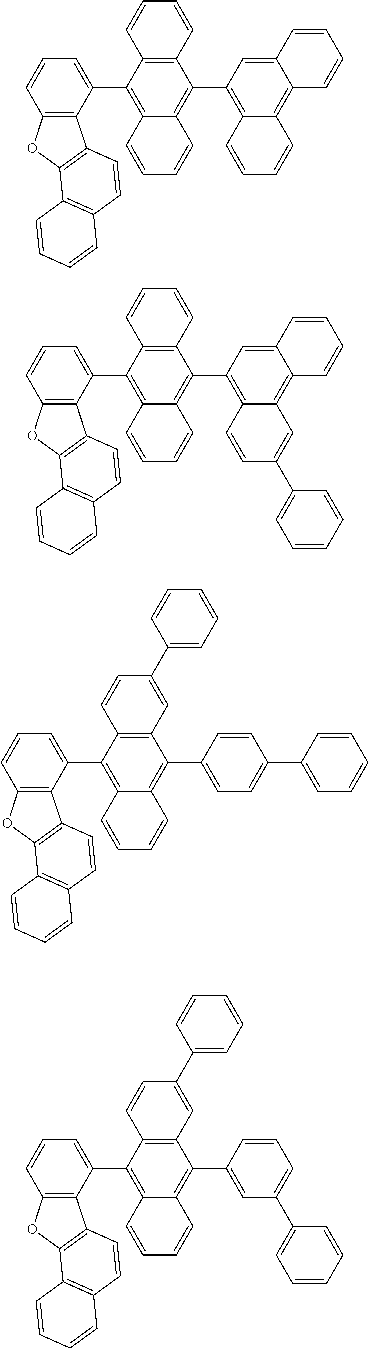 [SMILES: c1ccc2c(c1)cc(-c1c3ccccc3c(-c3cccc4oc5c6ccccc6ccc5c34)c3ccccc13)c1ccccc12 ; c1ccc(-c2ccc3c(-c4c5ccccc5c(-c5cccc6oc7c8ccccc8ccc7c56)c5ccccc45)cc4ccccc4c3c2)cc1 ; c1ccc(-c2ccc(-c3c4ccccc4c(-c4cccc5oc6c7ccccc7ccc6c45)c4ccc(-c5ccccc5)cc34)cc2)cc1 ; c1ccc(-c2cccc(-c3c4ccccc4c(-c4cccc5oc6c7ccccc7ccc6c45)c4ccc(-c5ccccc5)cc34)c2)cc1]

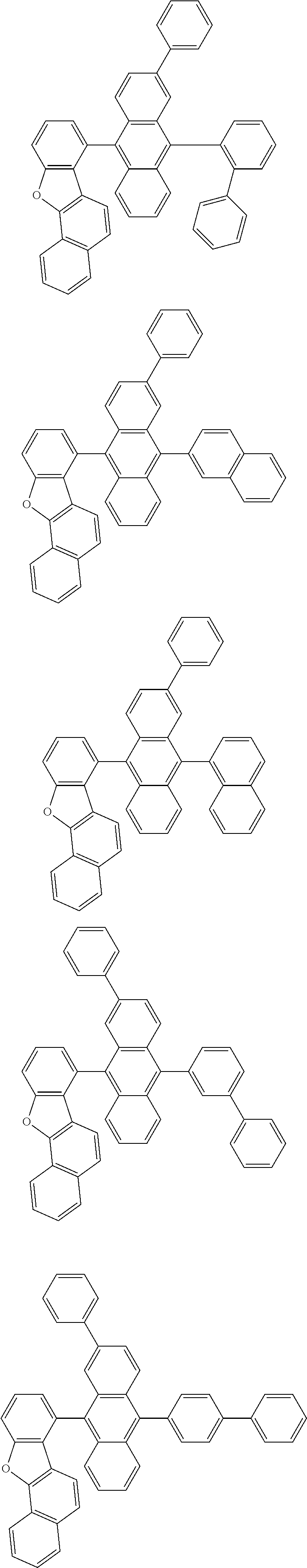

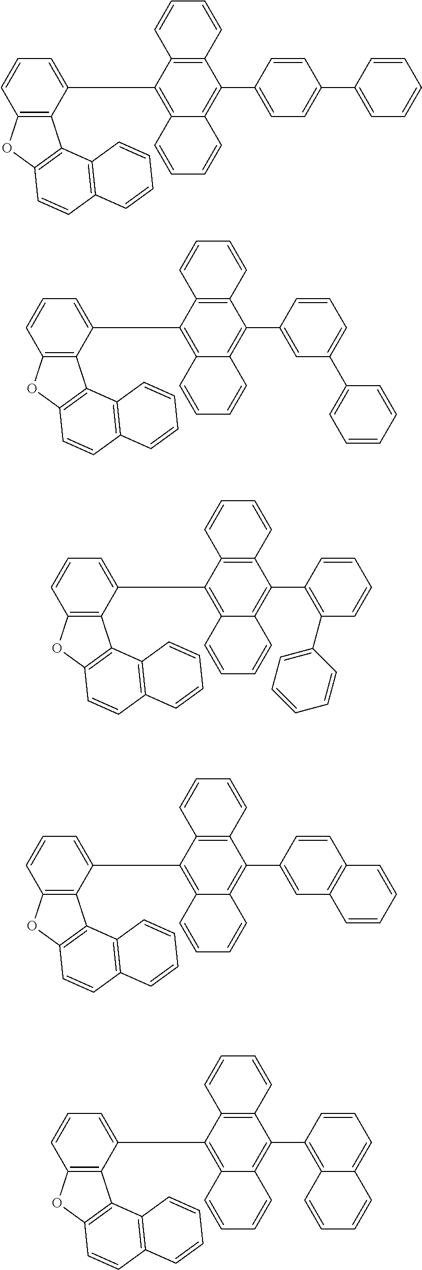 [SMILES: c1ccc(-c2ccc(-c3c4ccccc4c(-c4cccc5oc6ccc7ccccc7c6c45)c4ccccc34)cc2)cc1 ; c1ccc(-c2cccc(-c3c4ccccc4c(-c4cccc5oc6ccc7ccccc7c6c45)c4ccccc34)c2)cc1 ; c1ccc(-c2ccccc2-c2c3ccccc3c(-c3cccc4oc5ccc6ccccc6c5c34)c3ccccc23)cc1 ; c1ccc2cc(-c3c4ccccc4c(-c4cccc5oc6ccc7ccccc7c6c45)c4ccccc34)ccc2c1 ; c1ccc2c(-c3c4ccccc4c(-c4cccc5oc6ccc7ccccc7c6c45)c4ccccc34)cccc2c1]

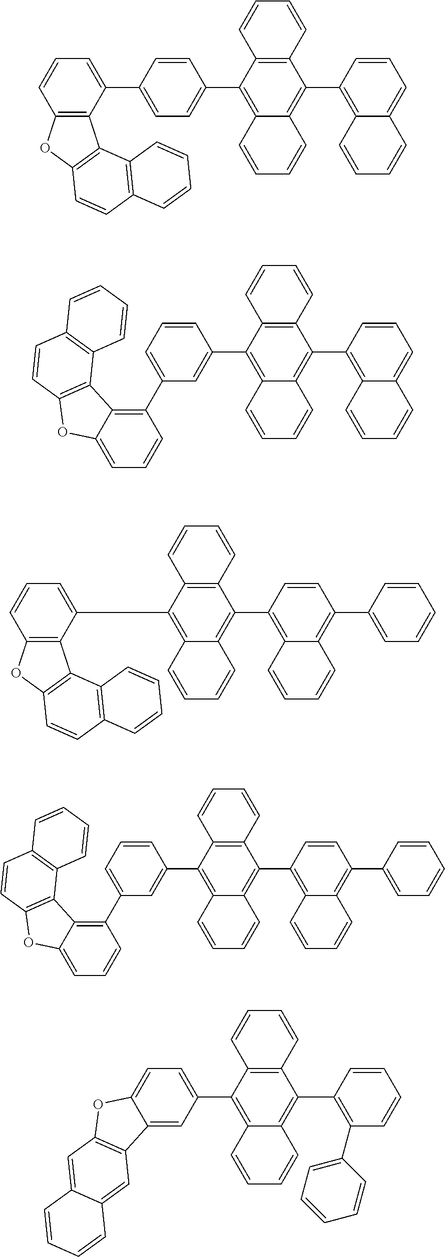 [SMILES: c1ccc2c(-c3c4ccccc4c(-c4ccc(-c5cccc6oc7ccc8ccccc8c7c56)cc4)c4ccccc34)cccc2c1 ; c1cc(-c2c3ccccc3c(-c3cccc4ccccc34)c3ccccc23)cc(-c2cccc3oc4ccc5ccccc5c4c23)c1 ; c1ccc(-c2ccc(-c3c4ccccc4c(-c4cccc5oc6ccc7ccccc7c6c45)c4ccccc34)c3ccccc23)cc1 ; c1ccc(-c2ccc(-c3c4ccccc4c(-c4cccc(-c5cccc6oc7ccc8ccccc8c7c56)c4)c4ccccc34)c3ccccc23)cc1 ; c1ccc(-c2ccccc2-c2c3ccccc3c(-c3ccc4oc5cc6ccccc6cc5c4c3)c3ccccc23)cc1]

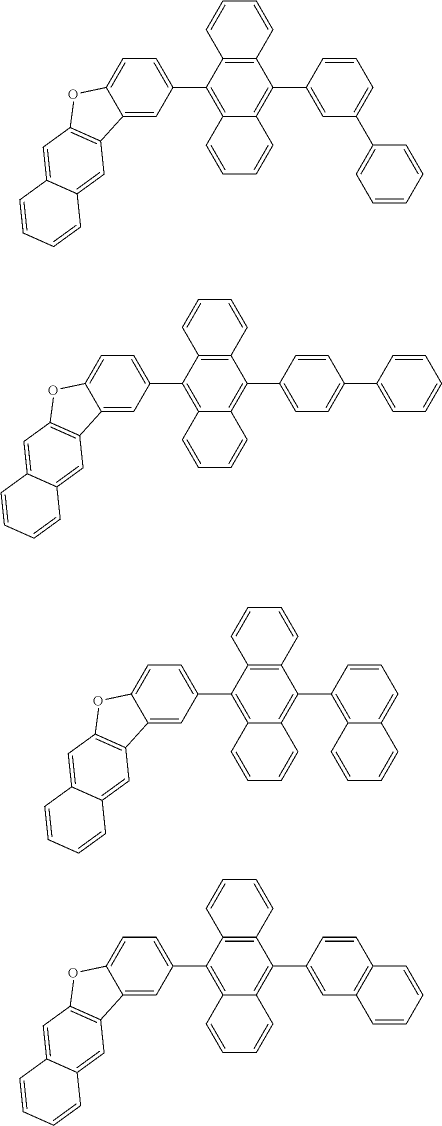 [SMILES: c1ccc(-c2cccc(-c3c4ccccc4c(-c4ccc5oc6cc7ccccc7cc6c5c4)c4ccccc34)c2)cc1 ; c1ccc(-c2ccc(-c3c4ccccc4c(-c4ccc5oc6cc7ccccc7cc6c5c4)c4ccccc34)cc2)cc1 ; c1ccc2cc3c(cc2c1)oc1ccc(-c2c4ccccc4c(-c4cccc5ccccc45)c4ccccc24)cc13 ; c1ccc2cc(-c3c4ccccc4c(-c4ccc5oc6cc7ccccc7cc6c5c4)c4ccccc34)ccc2c1]

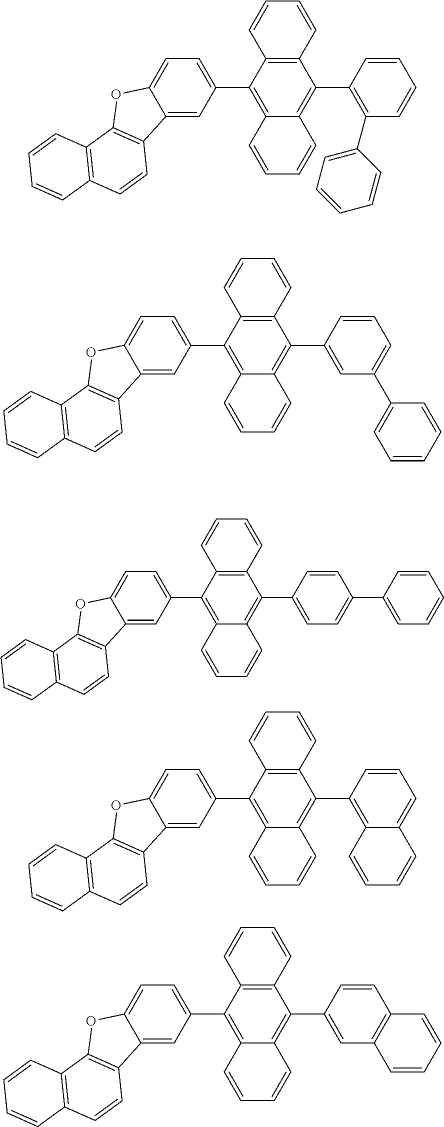

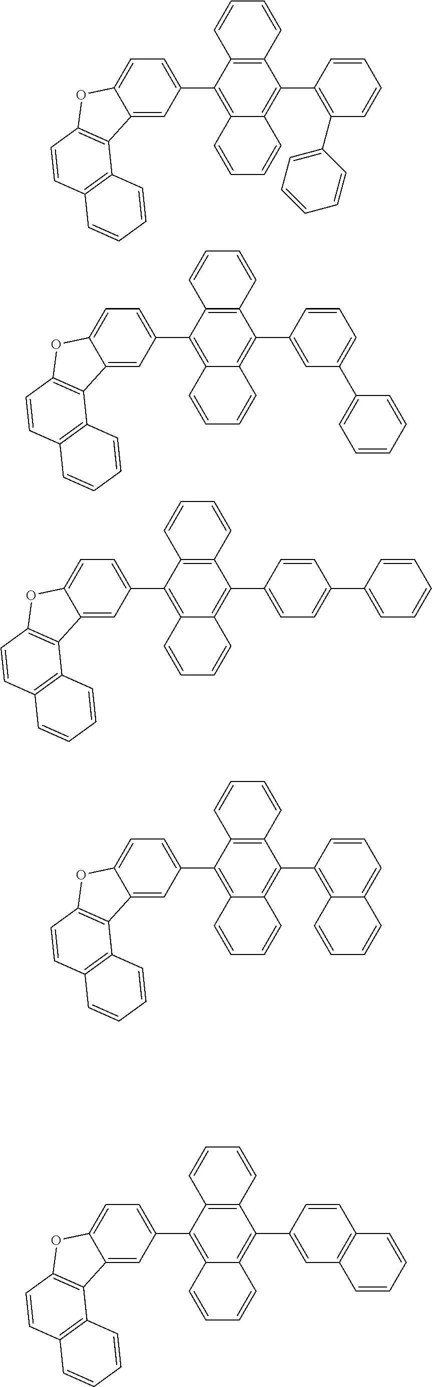 [SMILES: c1ccc(-c2ccccc2-c2c3ccccc3c(-c3ccc4oc5ccc6ccccc6c5c4c3)c3ccccc23)cc1 ; c1ccc(-c2cccc(-c3c4ccccc4c(-c4ccc5oc6ccc7ccccc7c6c5c4)c4ccccc34)c2)cc1 ; c1ccc(-c2ccc(-c3c4ccccc4c(-c4ccc5oc6ccc7ccccc7c6c5c4)c4ccccc34)cc2)cc1 ; c1ccc2c(-c3c4ccccc4c(-c4ccc5oc6ccc7ccccc7c6c5c4)c4ccccc34)cccc2c1 ; c1ccc2cc(-c3c4ccccc4c(-c4ccc5oc6ccc7ccccc7c6c5c4)c4ccccc34)ccc2c1]

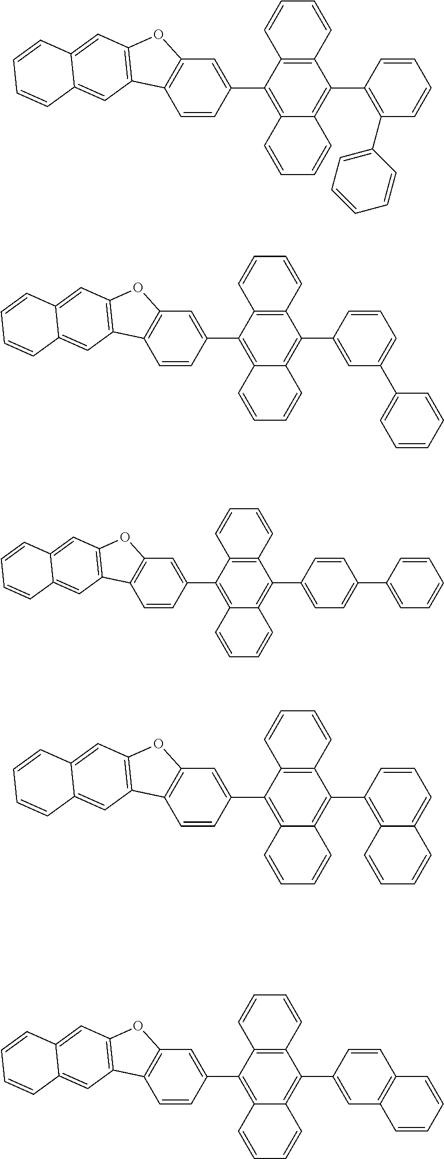 [SMILES: c1ccc(-c2ccccc2-c2c3ccccc3c(-c3ccc4c(c3)oc3cc5ccccc5cc34)c3ccccc23)cc1 ; c1ccc(-c2cccc(-c3c4ccccc4c(-c4ccc5c(c4)oc4cc6ccccc6cc45)c4ccccc34)c2)cc1 ; c1ccc(-c2ccc(-c3c4ccccc4c(-c4ccc5c(c4)oc4cc6ccccc6cc45)c4ccccc34)cc2)cc1 ; c1ccc2cc3c(cc2c1)oc1cc(-c2c4ccccc4c(-c4cccc5ccccc45)c4ccccc24)ccc13 ; c1ccc2cc(-c3c4ccccc4c(-c4ccc5c(c4)oc4cc6ccccc6cc45)c4ccccc34)ccc2c1]

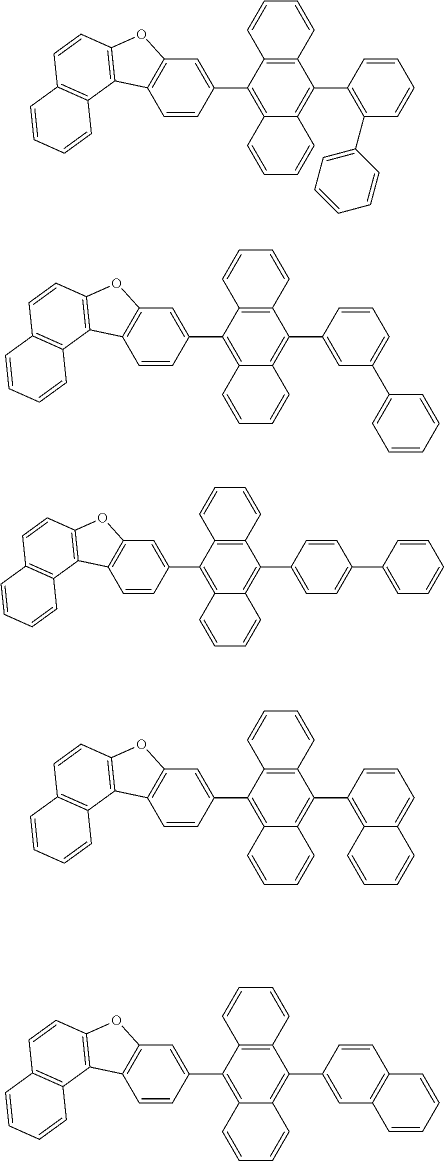 [SMILES: c1ccc(-c2ccccc2-c2c3ccccc3c(-c3ccc4c(c3)oc3ccc5ccccc5c34)c3ccccc23)cc1 ; c1ccc(-c2cccc(-c3c4ccccc4c(-c4ccc5c(c4)oc4ccc6ccccc6c45)c4ccccc34)c2)cc1 ; c1ccc(-c2ccc(-c3c4ccccc4c(-c4ccc5c(c4)oc4ccc6ccccc6c45)c4ccccc34)cc2)cc1 ; c1ccc2c(-c3c4ccccc4c(-c4ccc5c(c4)oc4ccc6ccccc6c45)c4ccccc34)cccc2c1 ; c1ccc2cc(-c3c4ccccc4c(-c4ccc5c(c4)oc4ccc6ccccc6c45)c4ccccc34)ccc2c1]

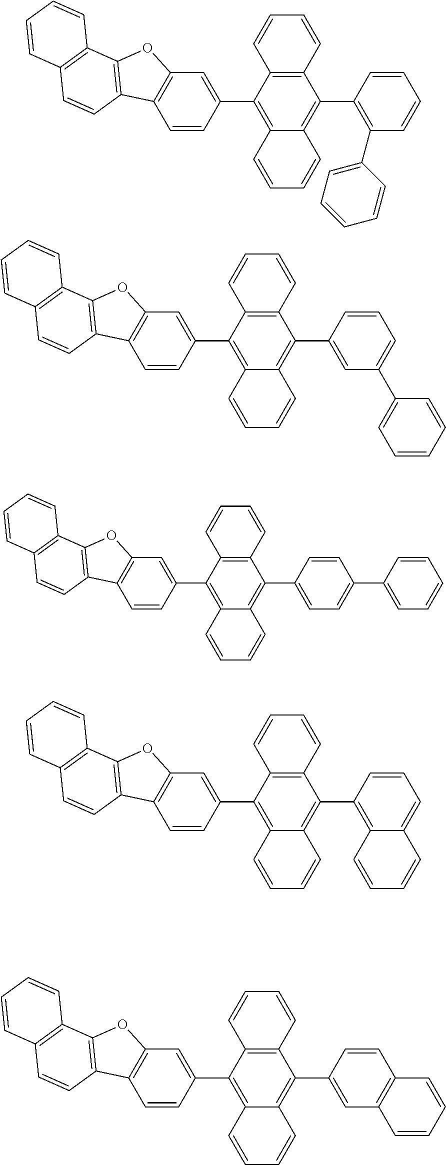 [SMILES: c1ccc(-c2ccccc2-c2c3ccccc3c(-c3ccc4c(c3)oc3c5ccccc5ccc43)c3ccccc23)cc1 ; c1ccc(-c2cccc(-c3c4ccccc4c(-c4ccc5c(c4)oc4c6ccccc6ccc54)c4ccccc34)c2)cc1 ; c1ccc(-c2ccc(-c3c4ccccc4c(-c4ccc5c(c4)oc4c6ccccc6ccc54)c4ccccc34)cc2)cc1 ; c1ccc2c(-c3c4ccccc4c(-c4ccc5c(c4)oc4c6ccccc6ccc54)c4ccccc34)cccc2c1 ; c1ccc2cc(-c3c4ccccc4c(-c4ccc5c(c4)oc4c6ccccc6ccc54)c4ccccc34)ccc2c1]

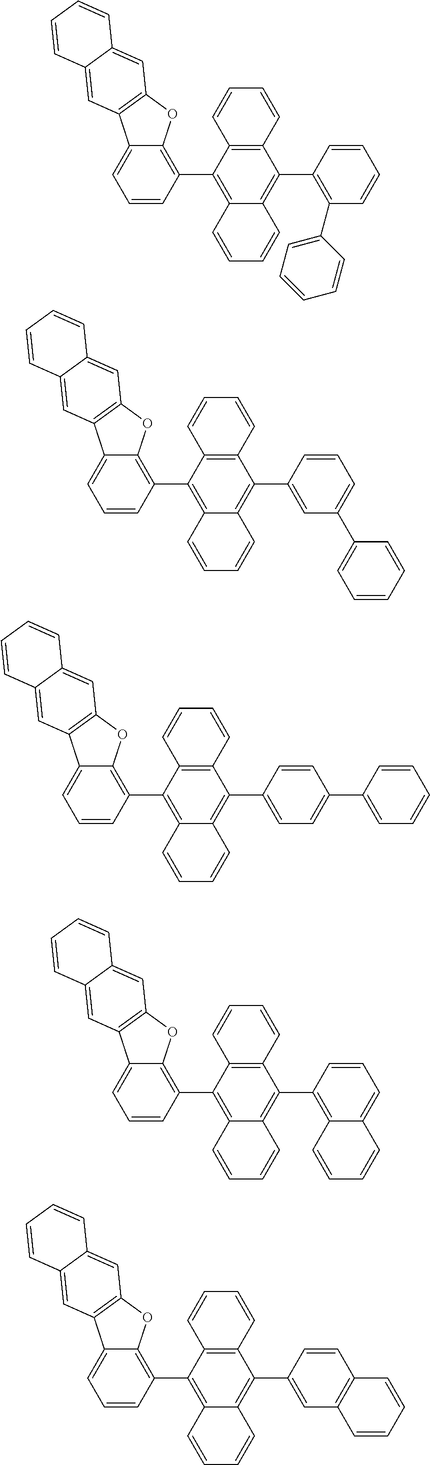 [SMILES: c1ccc(-c2ccccc2-c2c3ccccc3c(-c3cccc4c3oc3cc5ccccc5cc34)c3ccccc23)cc1 ; c1ccc(-c2cccc(-c3c4ccccc4c(-c4cccc5c4oc4cc6ccccc6cc45)c4ccccc34)c2)cc1 ; c1ccc(-c2ccc(-c3c4ccccc4c(-c4cccc5c4oc4cc6ccccc6cc45)c4ccccc34)cc2)cc1 ; c1ccc2cc3c(cc2c1)oc1c(-c2c4ccccc4c(-c4cccc5ccccc45)c4ccccc24)cccc13 ; c1ccc2cc(-c3c4ccccc4c(-c4cccc5c4oc4cc6ccccc6cc45)c4ccccc34)ccc2c1]

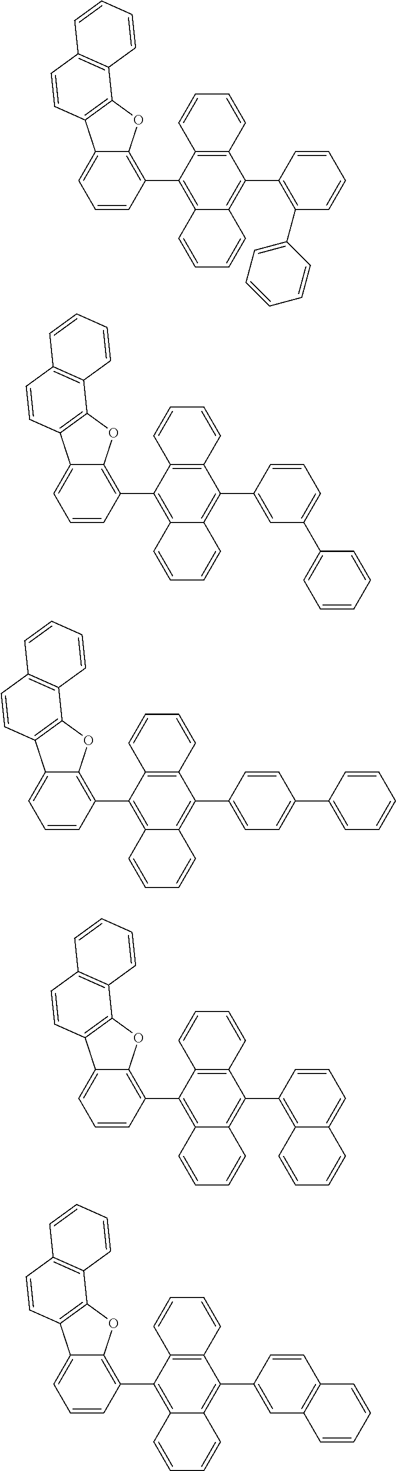 [SMILES: c1ccc(-c2ccccc2-c2c3ccccc3c(-c3cccc4c3oc3c5ccccc5ccc43)c3ccccc23)cc1 ; c1ccc(-c2cccc(-c3c4ccccc4c(-c4cccc5c4oc4c6ccccc6ccc54)c4ccccc34)c2)cc1 ; c1ccc(-c2ccc(-c3c4ccccc4c(-c4cccc5c4oc4c6ccccc6ccc54)c4ccccc34)cc2)cc1 ; c1ccc2c(-c3c4ccccc4c(-c4cccc5c4oc4c6ccccc6ccc54)c4ccccc34)cccc2c1 ; c1ccc2cc(-c3c4ccccc4c(-c4cccc5c4oc4c6ccccc6ccc54)c4ccccc34)ccc2c1]

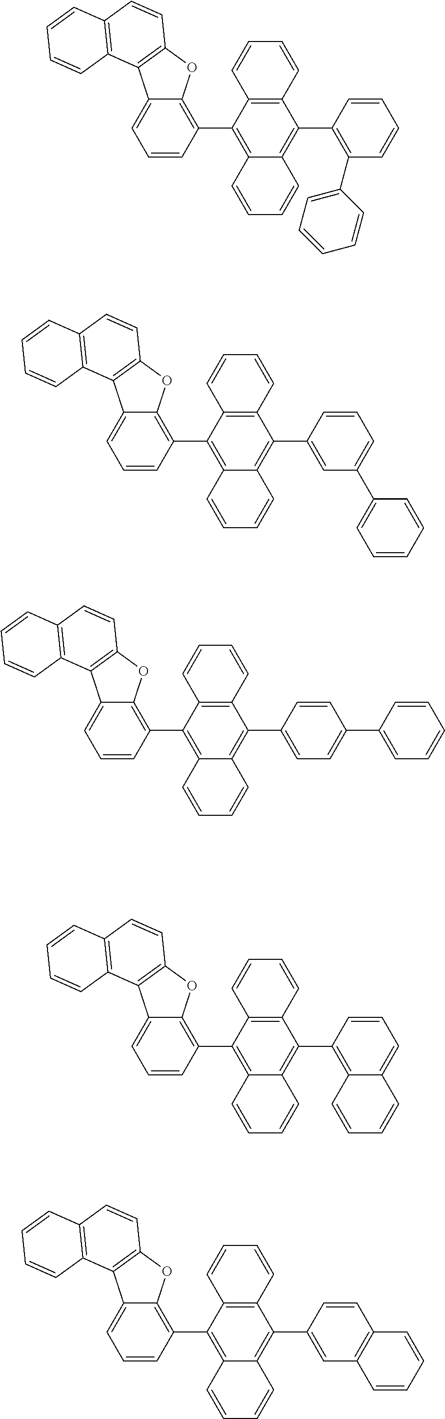 [SMILES: c1ccc(-c2ccccc2-c2c3ccccc3c(-c3cccc4c3oc3ccc5ccccc5c34)c3ccccc23)cc1 ; c1ccc(-c2cccc(-c3c4ccccc4c(-c4cccc5c4oc4ccc6ccccc6c45)c4ccccc34)c2)cc1 ; c1ccc(-c2ccc(-c3c4ccccc4c(-c4cccc5c4oc4ccc6ccccc6c45)c4ccccc34)cc2)cc1 ; c1ccc2c(-c3c4ccccc4c(-c4cccc5c4oc4ccc6ccccc6c45)c4ccccc34)cccc2c1 ; c1ccc2cc(-c3c4ccccc4c(-c4cccc5c4oc4ccc6ccccc6c45)c4ccccc34)ccc2c1]

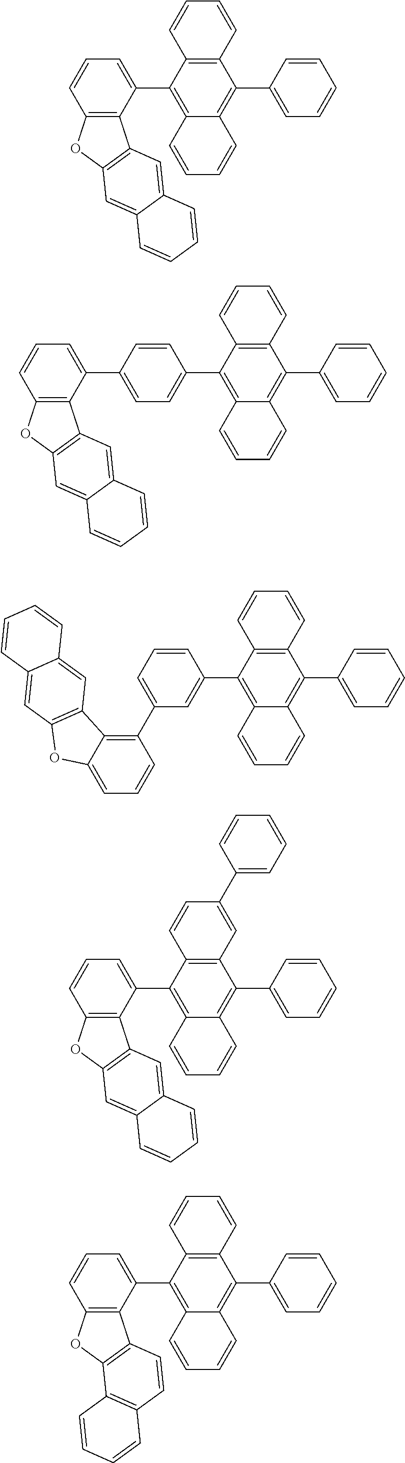

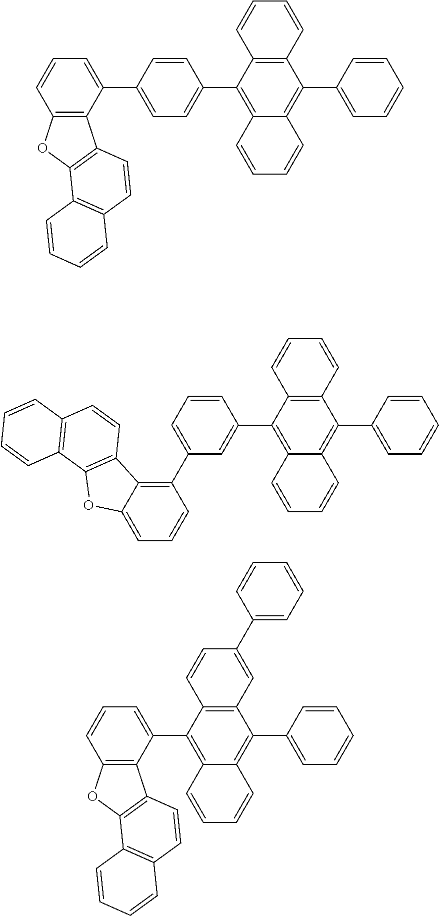 [SMILES: c1ccc(-c2c3ccccc3c(-c3ccc(-c4cccc5oc6c7ccccc7ccc6c45)cc3)c3ccccc23)cc1 ; c1ccc(-c2c3ccccc3c(-c3cccc(-c4cccc5oc6c7ccccc7ccc6c45)c3)c3ccccc23)cc1 ; c1ccc(-c2ccc3c(-c4cccc5oc6c7ccccc7ccc6c45)c4ccccc4c(-c4ccccc4)c3c2)cc1]

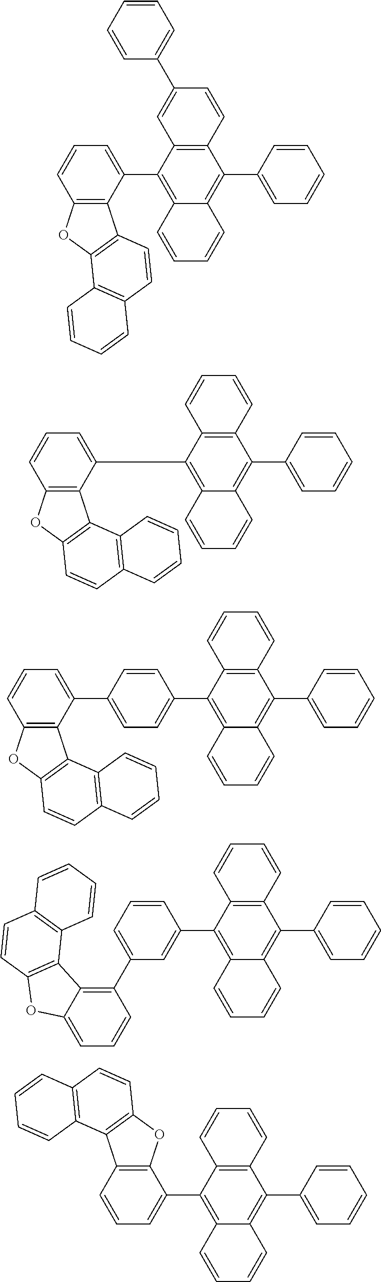

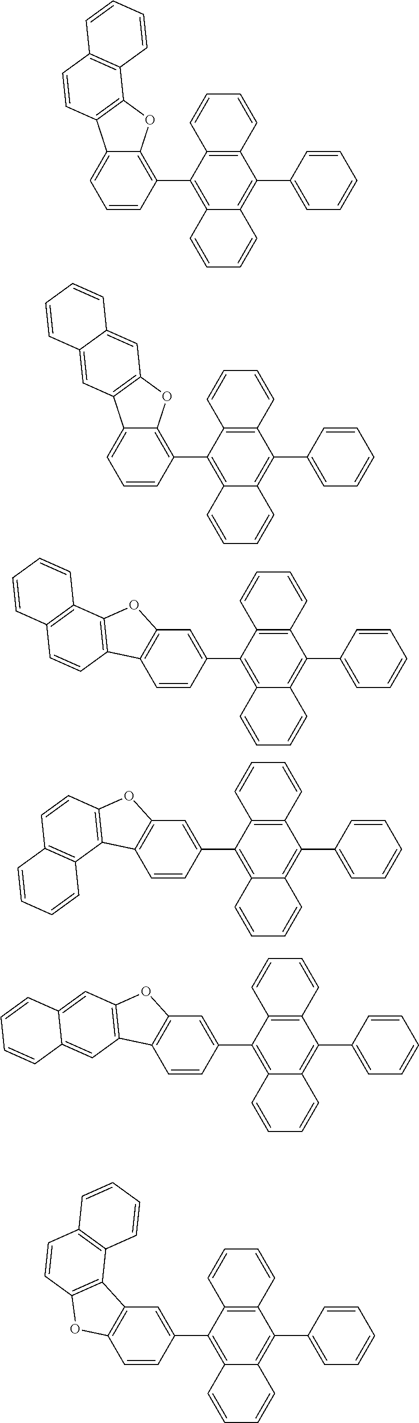

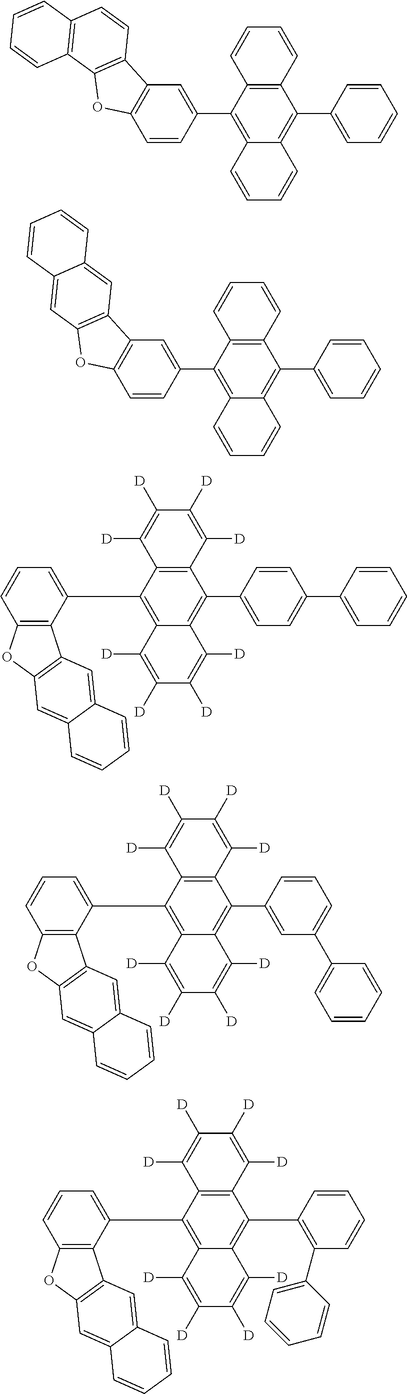

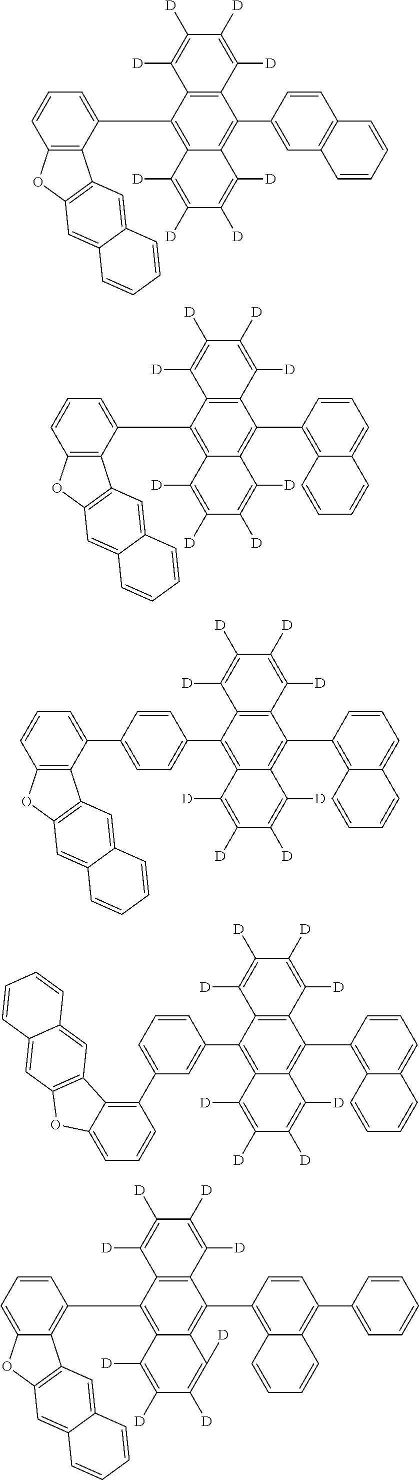

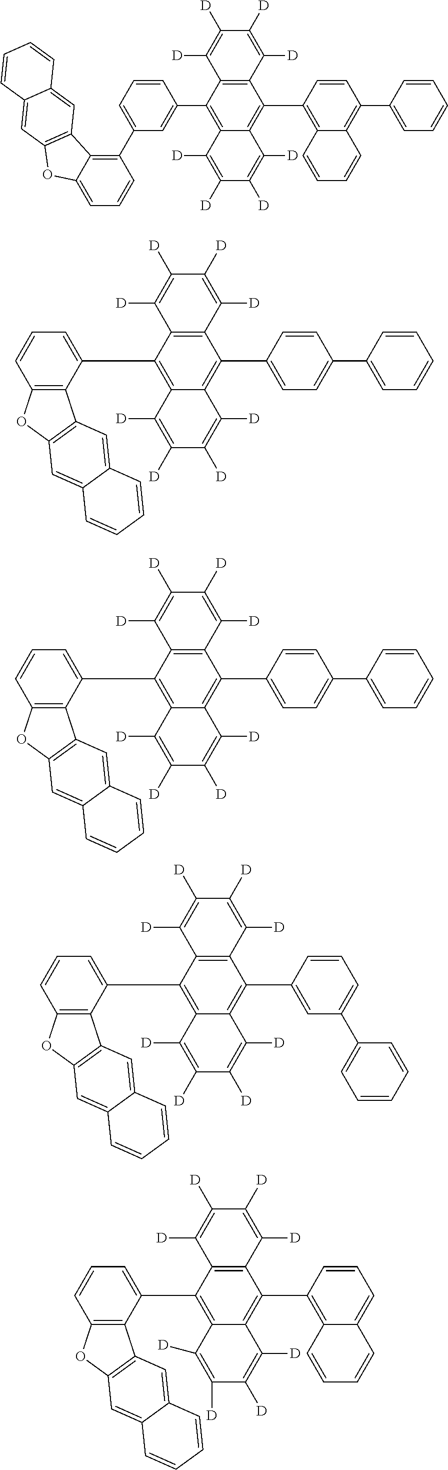

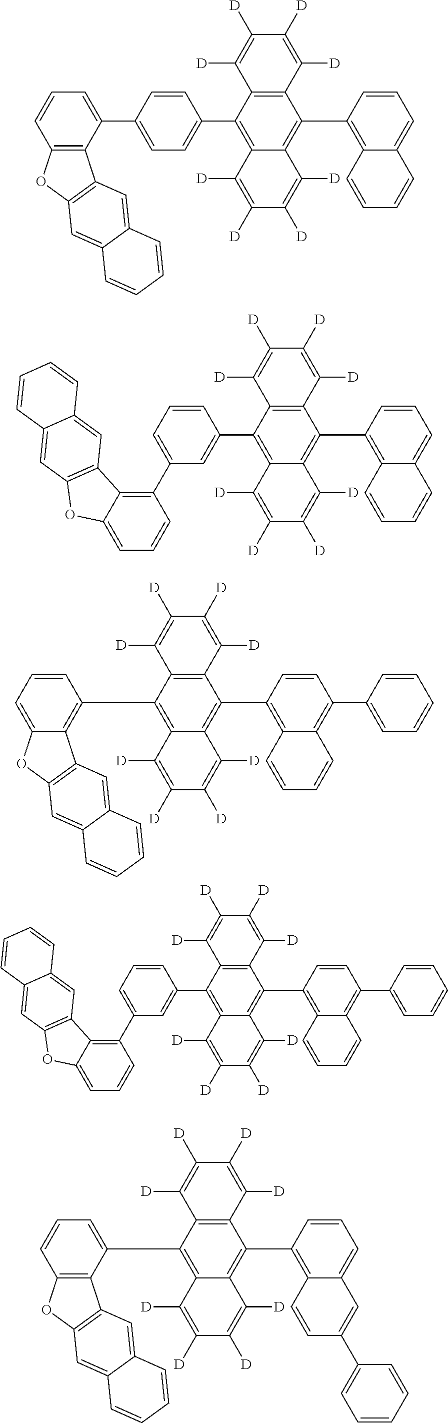

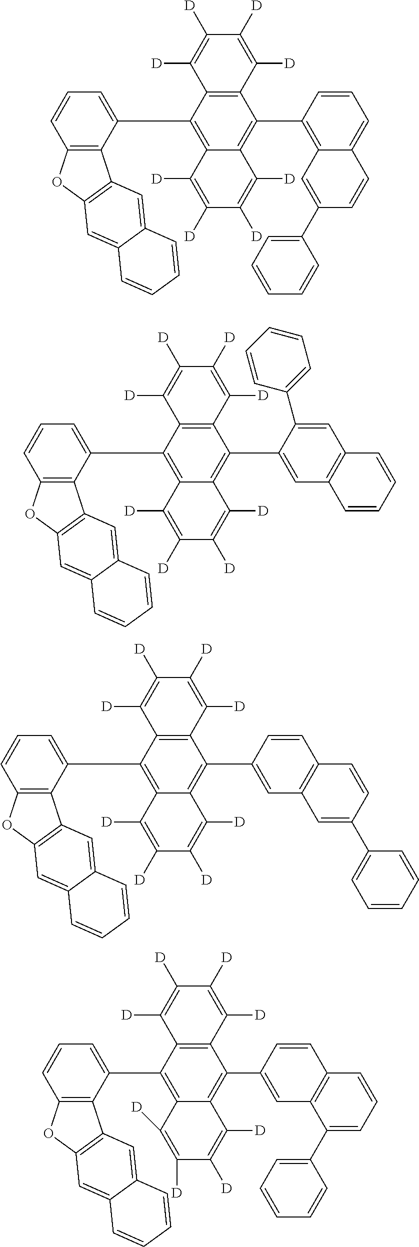

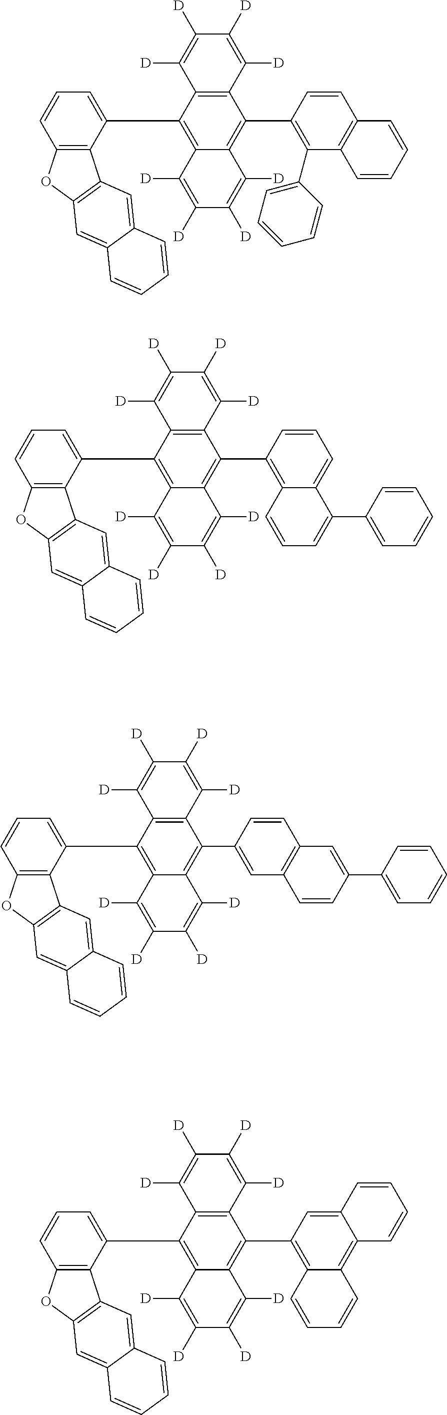 [SMILES: [2H]c1c([2H])c([2H])c2c(-c3cccc4oc5cc6ccccc6cc5c34)c3c([2H])c([2H])c([2H])c([2H])c3c(-c3ccc4ccccc4c3-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3cccc4c(-c5ccccc5)cccc34)c2c1[2H] ; [2H]c1c([2H])c([2H])c2c(-c3cccc4oc5cc6ccccc6cc5c34)c3c([2H])c([2H])c([2H])c([2H])c3c(-c3ccc4cc(-c5ccccc5)ccc4c3)c2c1[2H] ; [2H]c1c([2H])c([2H])c2c(-c3cccc4oc5cc6ccccc6cc5c34)c3c([2H])c([2H])c([2H])c([2H])c3c(-c3cc4ccccc4c4ccccc34)c2c1[2H]]

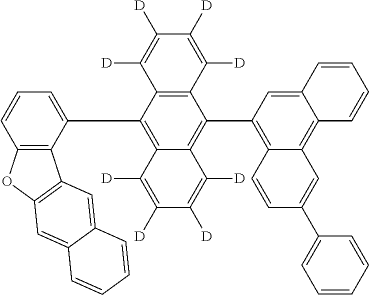 [SMILES: [2H]c1c([2H])c([2H])c2c(-c3cccc4oc5cc6ccccc6cc5c34)c3c([2H])c([2H])c([2H])c([2H])c3c(-c3cc4ccccc4c4cc(-c5ccccc5)ccc34)c2c1[2H]]

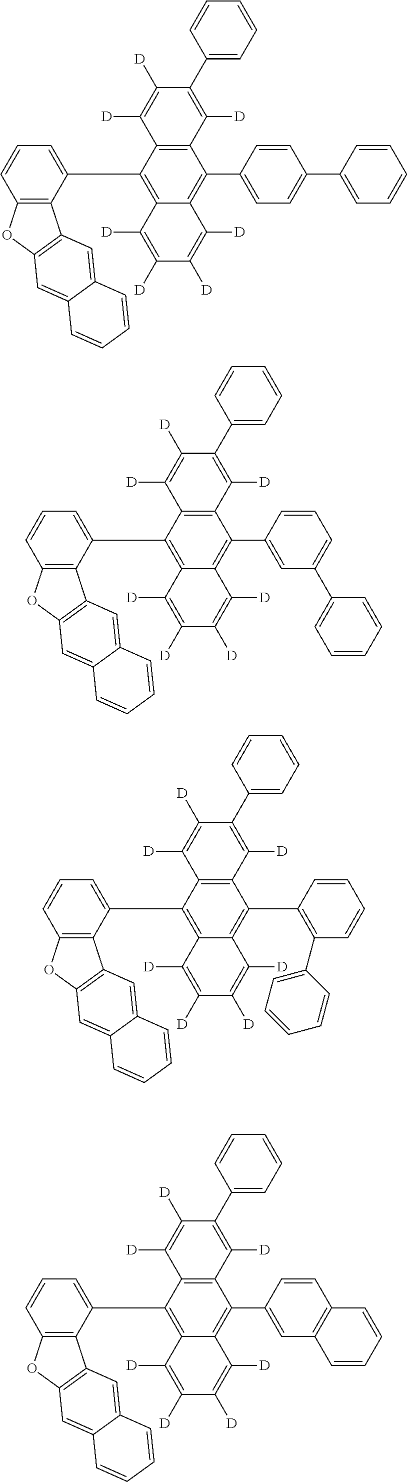 [SMILES: [2H]c1c([2H])c([2H])c2c(-c3cccc4oc5cc6ccccc6cc5c34)c3c([2H])c([2H])c(-c4ccccc4)c([2H])c3c(-c3ccc(-c4ccccc4)cc3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3cccc(-c4ccccc4)c3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3ccccc3-c3ccccc3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3ccc4ccccc4c3)c2c1[2H]]

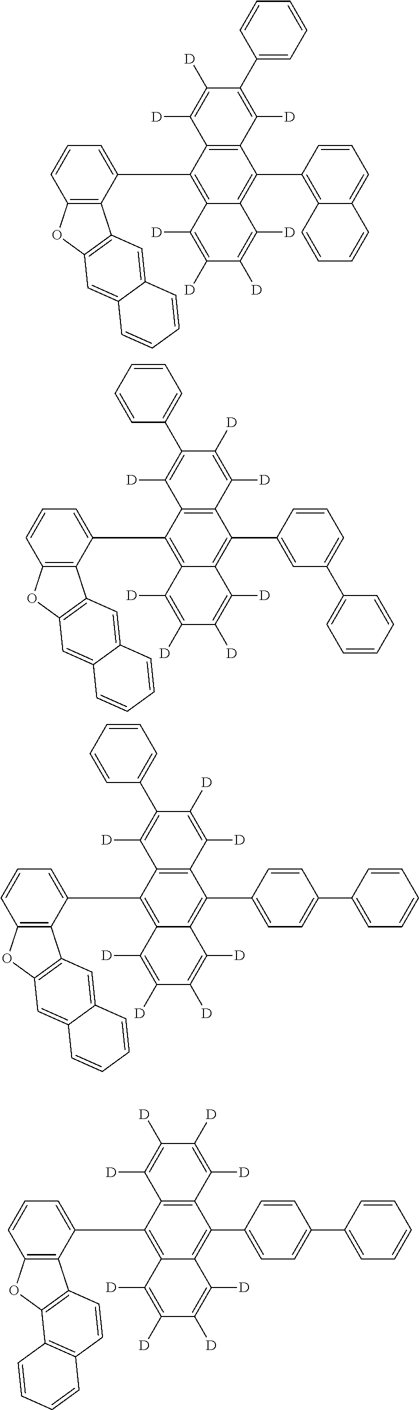

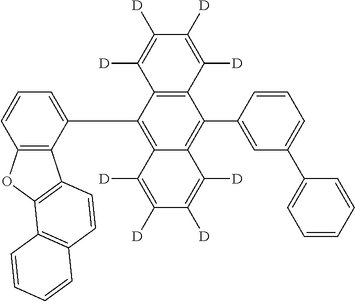 [SMILES: [2H]c1c([2H])c([2H])c2c(-c3cccc4oc5c6ccccc6ccc5c34)c3c([2H])c([2H])c([2H])c([2H])c3c(-c3cccc(-c4ccccc4)c3)c2c1[2H]]

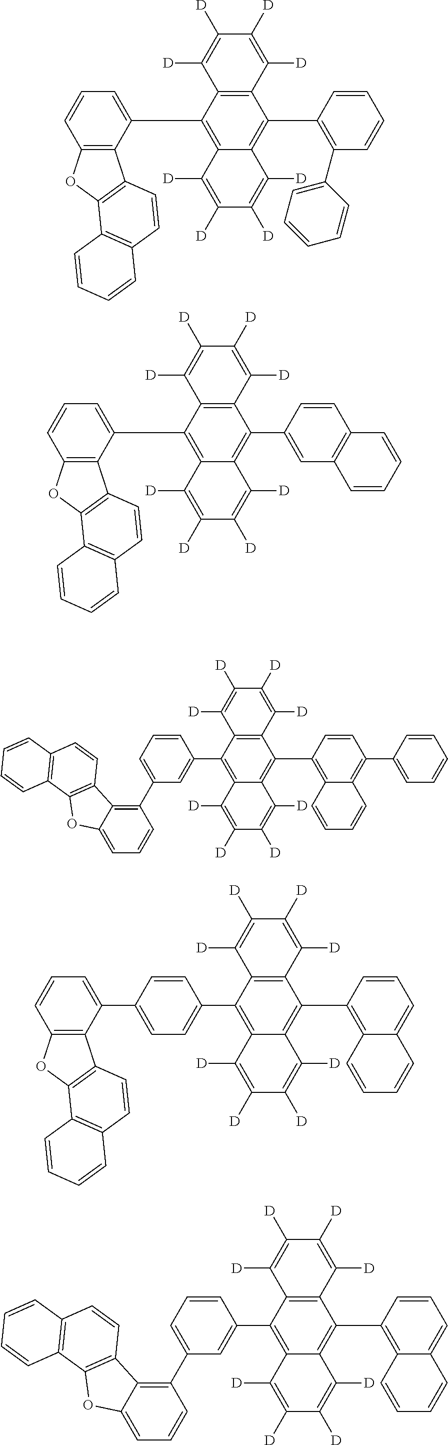

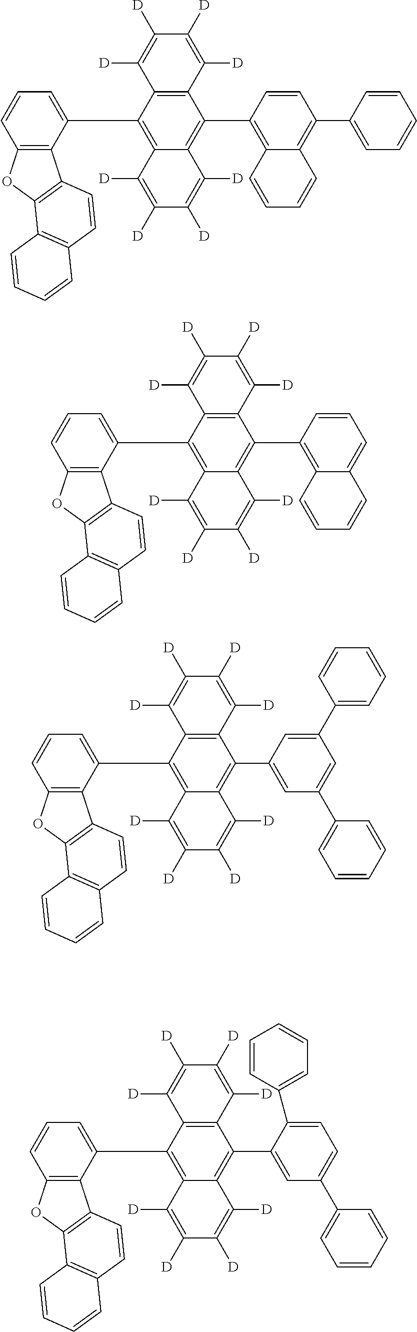 [SMILES: [2H]c1c([2H])c([2H])c2c(-c3cccc4oc5c6ccccc6ccc5c34)c3c([2H])c([2H])c([2H])c([2H])c3c(-c3ccc(-c4ccccc4)c4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cccc4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3cccc4oc5c6ccccc6ccc5c34)c3c([2H])c([2H])c([2H])c([2H])c3c(-c3cc(-c4ccccc4)ccc3-c3ccccc3)c2c1[2H]]

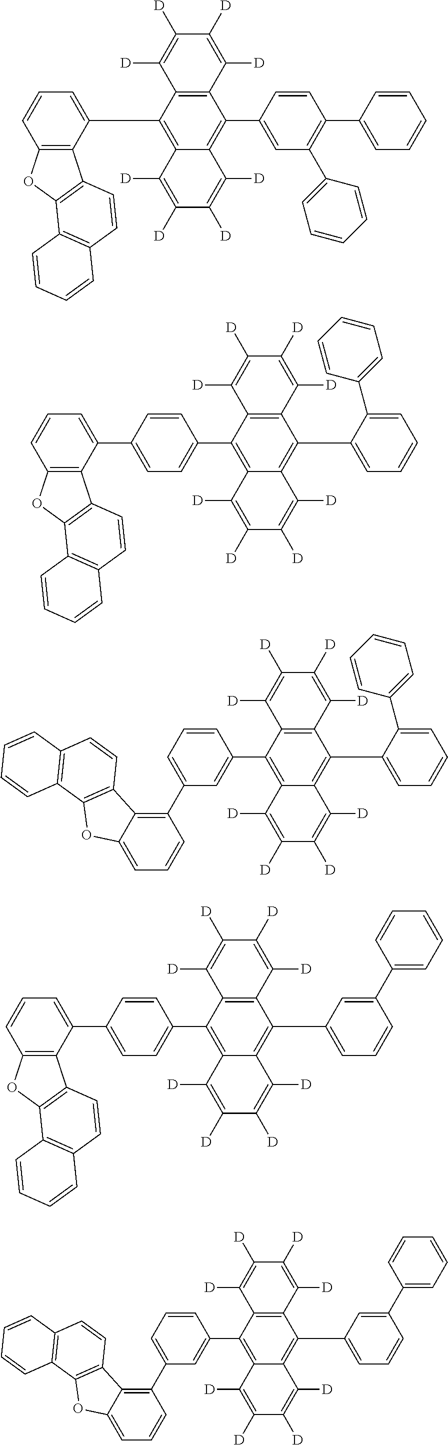

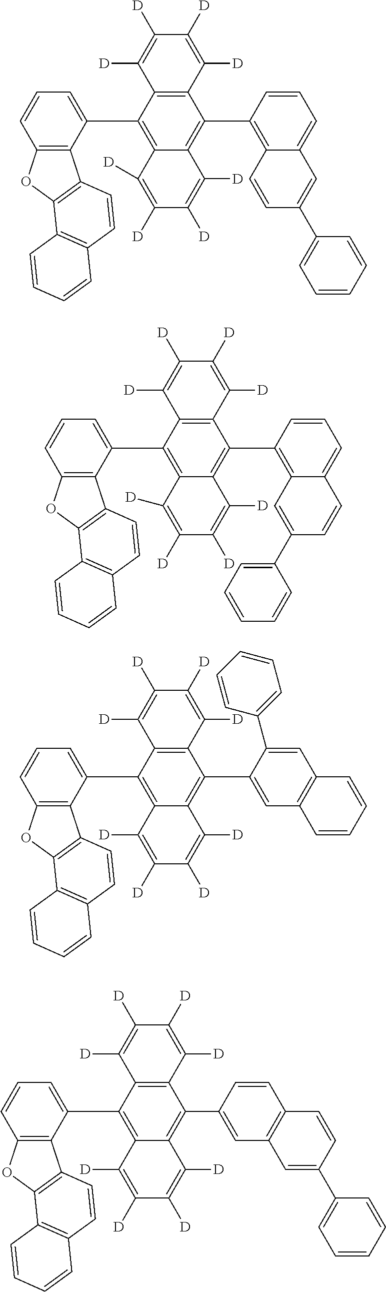

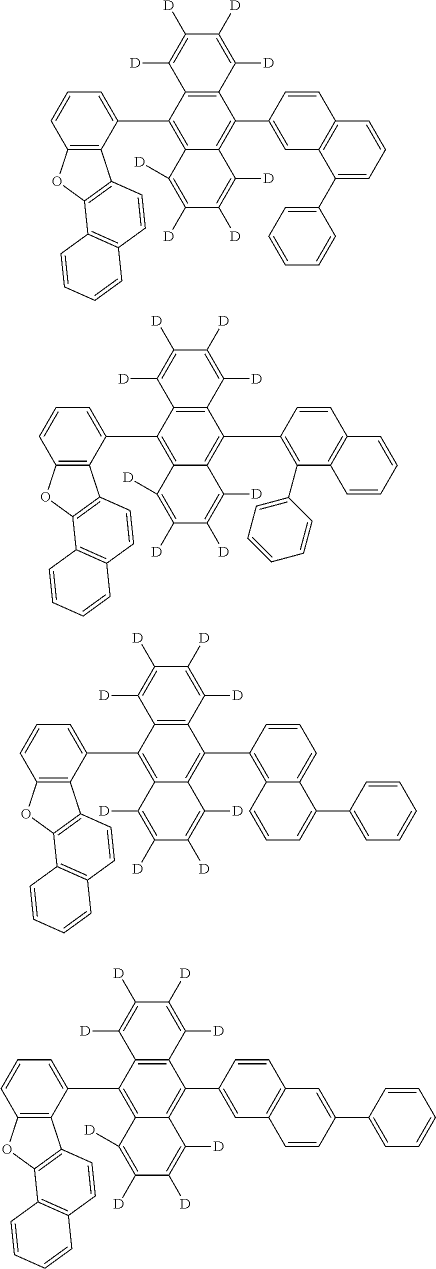 [SMILES: [2H]c1c([2H])c([2H])c2c(-c3cccc4oc5c6ccccc6ccc5c34)c3c([2H])c([2H])c([2H])c([2H])c3c(-c3ccc4cccc(-c5ccccc5)c4c3)c2c1[2H] ; [2H]c1c([2H])c([2H])c2c(-c3cccc4oc5c6ccccc6ccc5c34)c3c([2H])c([2H])c([2H])c([2H])c3c(-c3ccc4ccccc4c3-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3cccc4c(-c5ccccc5)cccc34)c2c1[2H] ; [2H]c1c([2H])c([2H])c2c(-c3cccc4oc5c6ccccc6ccc5c34)c3c([2H])c([2H])c([2H])c([2H])c3c(-c3ccc4cc(-c5ccccc5)ccc4c3)c2c1[2H]]

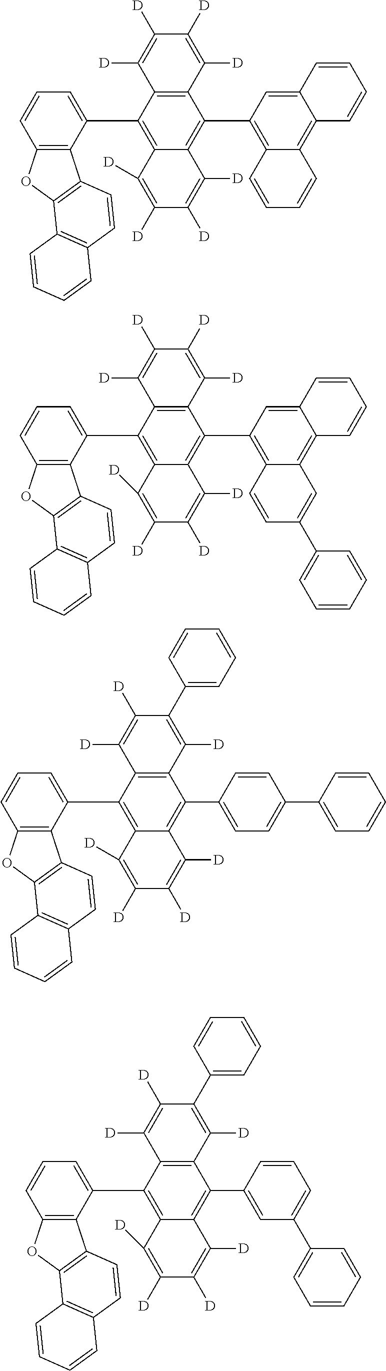 [SMILES: [2H]c1c([2H])c([2H])c2c(-c3cccc4oc5c6ccccc6ccc5c34)c3c([2H])c([2H])c([2H])c([2H])c3c(-c3cc4ccccc4c4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cc4ccccc4c4cc(-c5ccccc5)ccc34)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccc(-c4ccccc4)cc3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3cccc(-c4ccccc4)c3)c2c1[2H]]

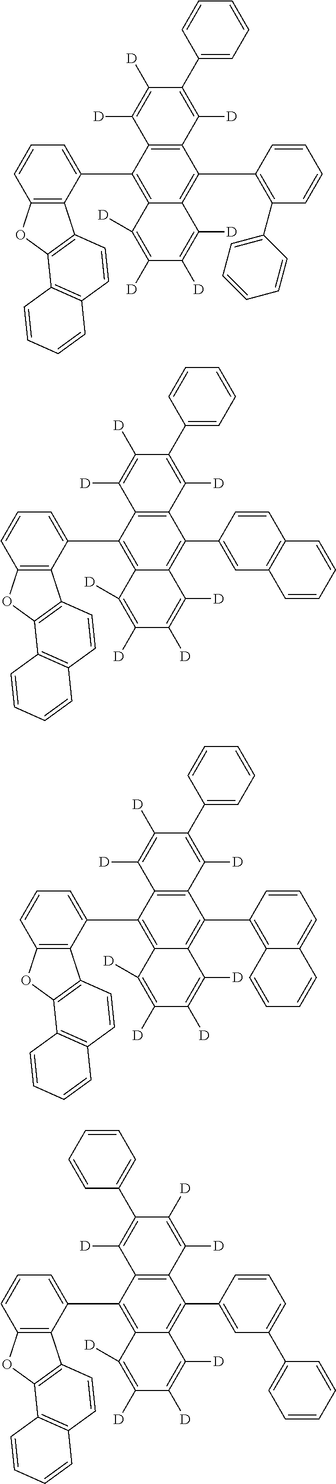

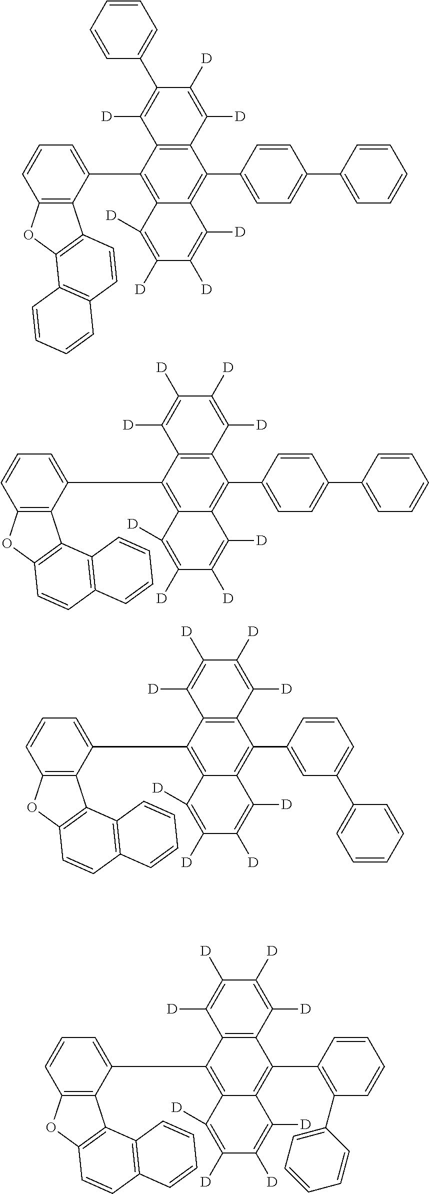

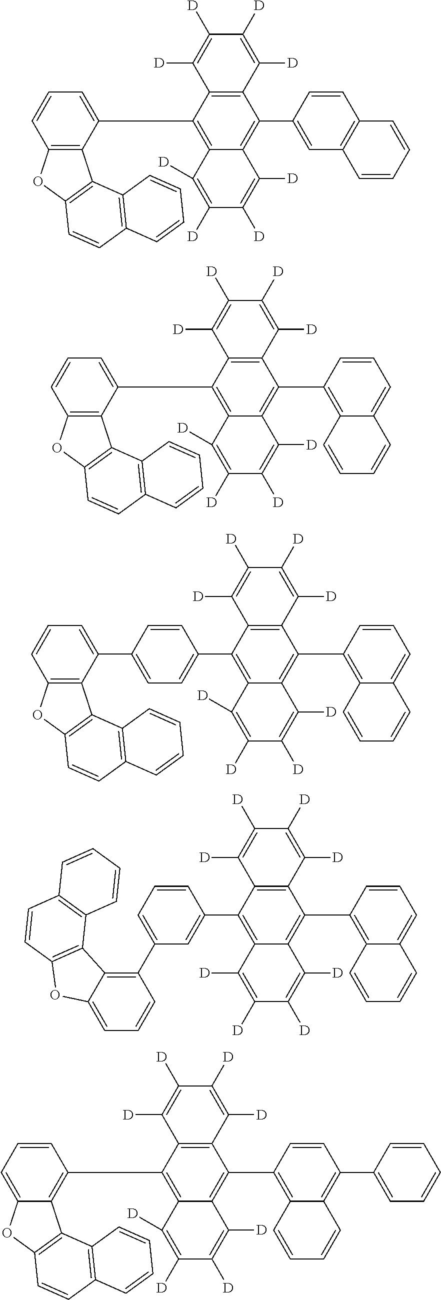

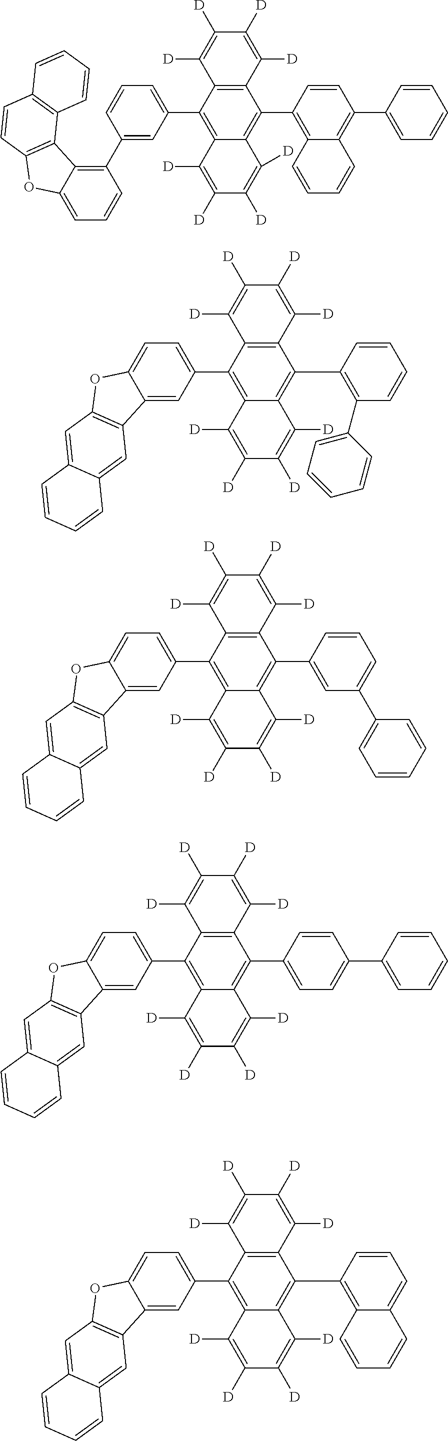

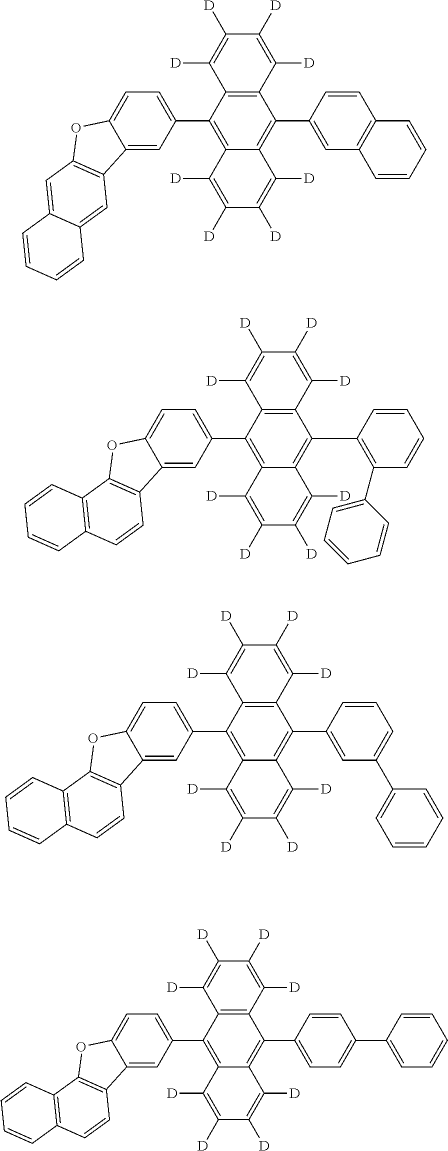 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccccc3-c3ccccc3)c3c([2H])c([2H])c([2H])c([2H])c3c(-c3ccc4oc5c6ccccc6ccc5c4c3)c2c1[2H] ; [2H]c1c([2H])c([2H])c2c(-c3ccc4oc5c6ccccc6ccc5c4c3)c3c([2H])c([2H])c([2H])c([2H])c3c(-c3cccc(-c4ccccc4)c3)c2c1[2H] ; [2H]c1c([2H])c([2H])c2c(-c3ccc4oc5c6ccccc6ccc5c4c3)c3c([2H])c([2H])c([2H])c([2H])c3c(-c3ccc(-c4ccccc4)cc3)c2c1[2H]]

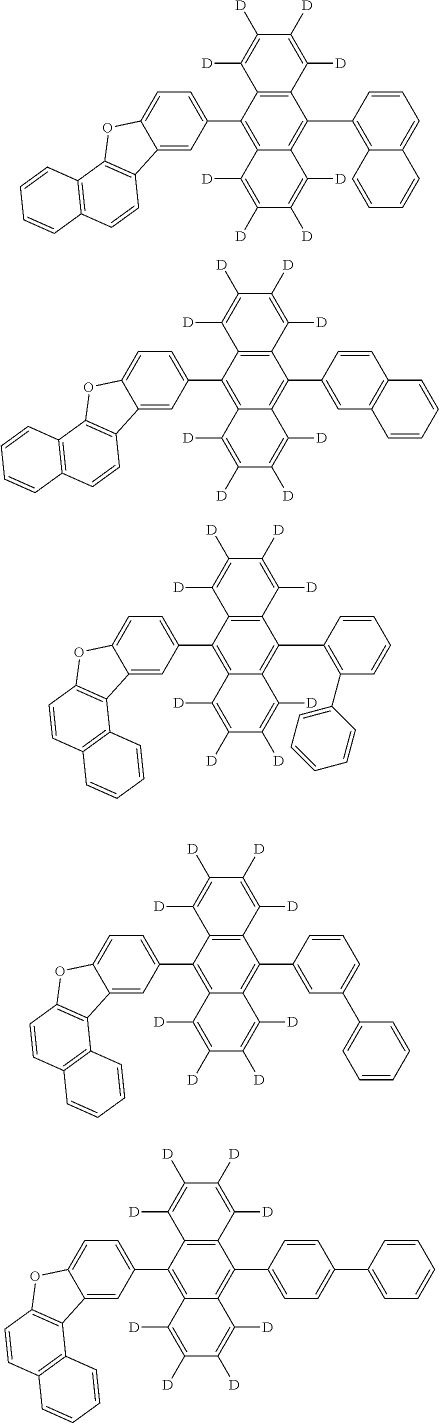

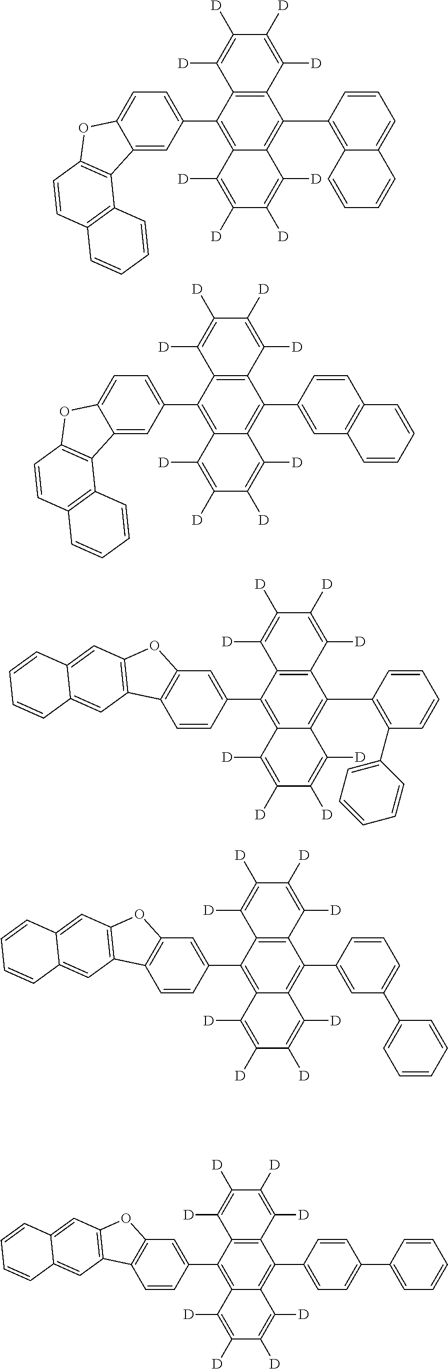

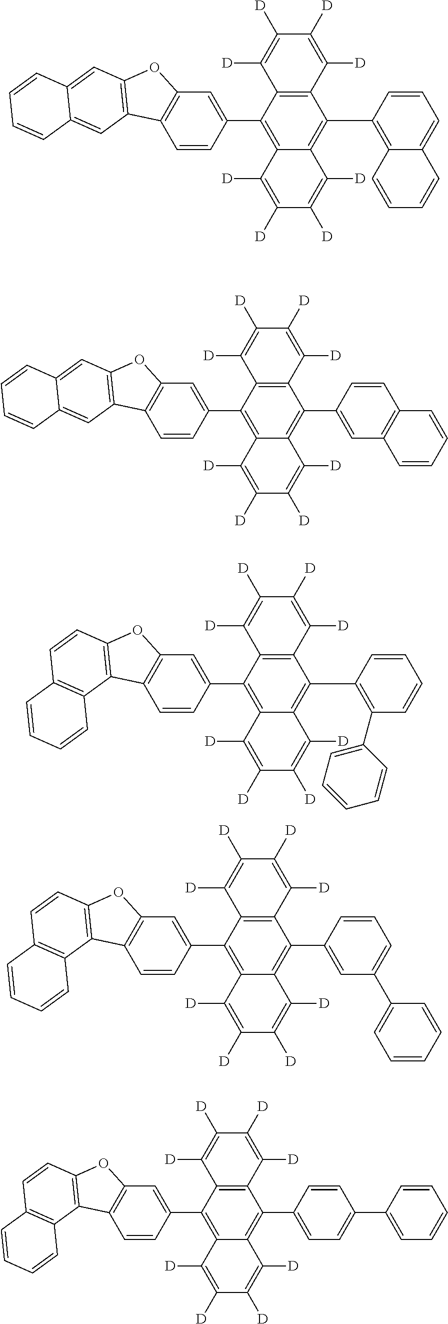

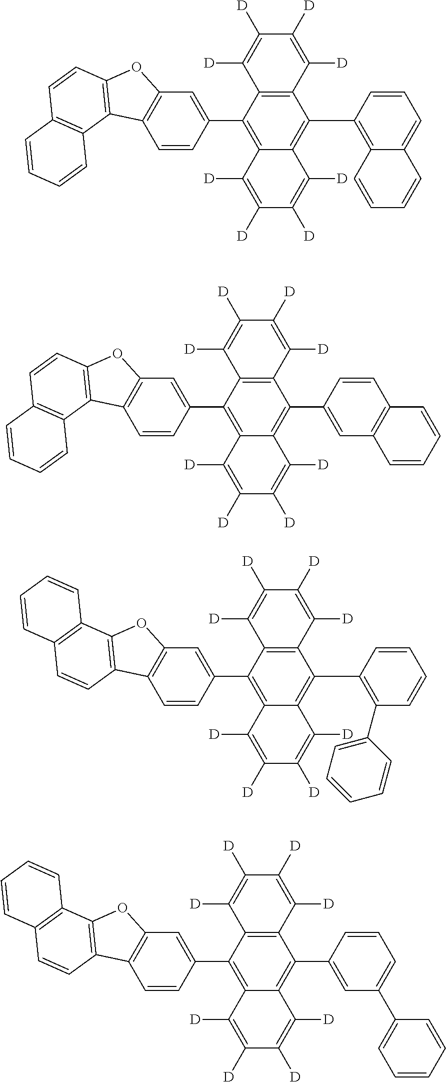

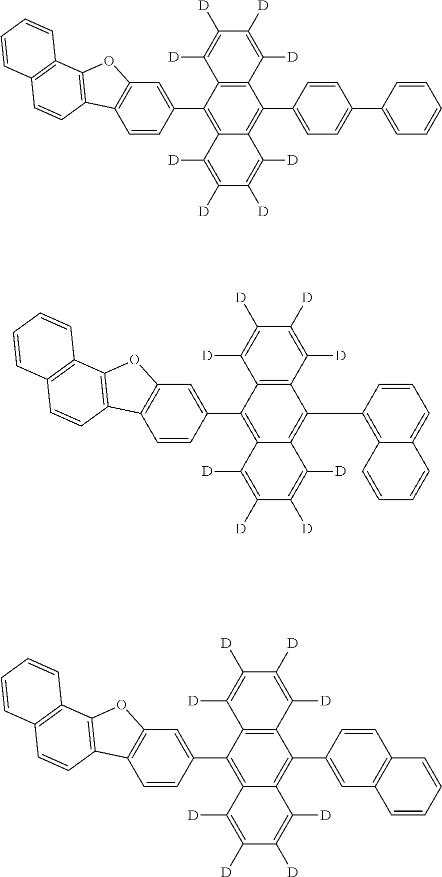 [SMILES: [2H]c1c([2H])c([2H])c2c(-c3ccc4c(c3)oc3c5ccccc5ccc43)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4c(c3)oc3c5ccccc5ccc43)c2c1[2H] ; [2H]c1c([2H])c([2H])c2c(-c3ccc4c(c3)oc3c5ccccc5ccc43)c3c([2H])c([2H])c([2H])c([2H])c3c(-c3ccc4ccccc4c3)c2c1[2H]]

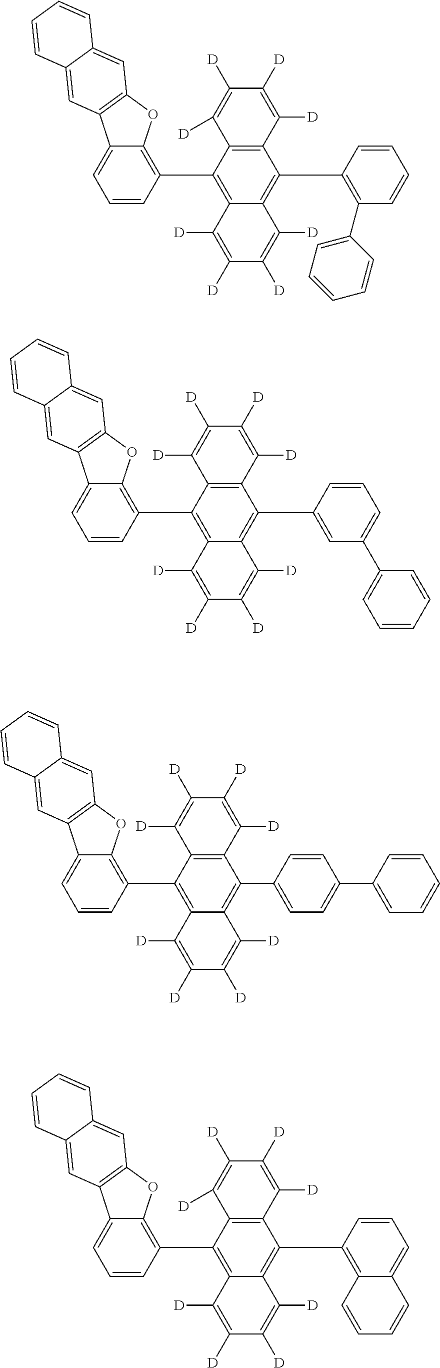 [SMILES: [2H]c1c([2H])c([2H])c2c(-c3cccc4c3oc3cc5ccccc5cc34)c3c([2H])c([2H])c([2H])c([2H])c3c(-c3ccccc3-c3ccccc3)c2c1[2H] ; [2H]c1c([2H])c([2H])c2c(-c3cccc4c3oc3cc5ccccc5cc34)c3c([2H])c([2H])c([2H])c([2H])c3c(-c3cccc(-c4ccccc4)c3)c2c1[2H] ; [2H]c1c([2H])c([2H])c2c(-c3cccc4c3oc3cc5ccccc5cc34)c3c([2H])c([2H])c([2H])c([2H])c3c(-c3ccc(-c4ccccc4)cc3)c2c1[2H] ; [2H]c1c([2H])c([2H])c2c(-c3cccc4c3oc3cc5ccccc5cc34)c3c([2H])c([2H])c([2H])c([2H])c3c(-c3cccc4ccccc34)c2c1[2H]]

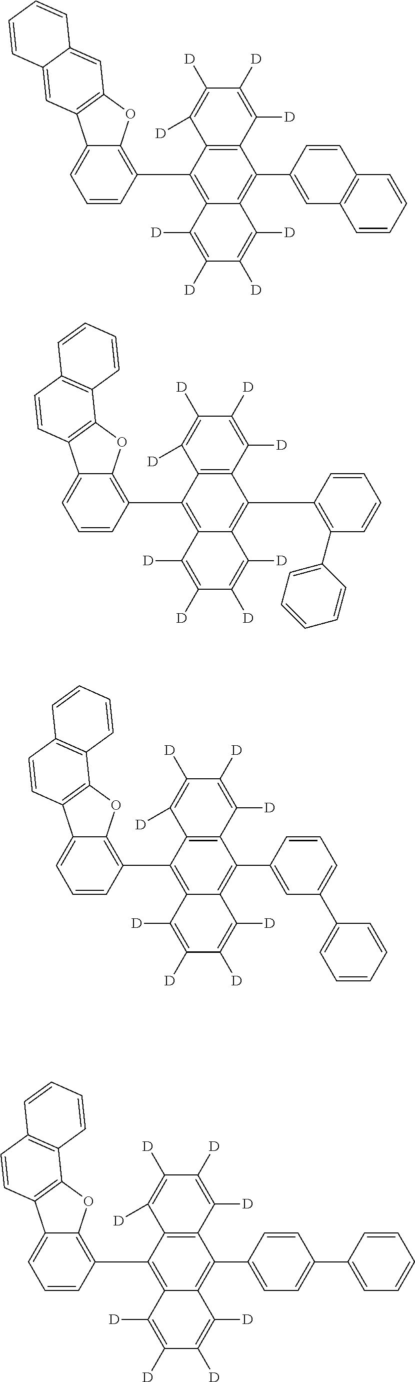 [SMILES: [2H]c1c([2H])c([2H])c2c(-c3cccc4c3oc3cc5ccccc5cc34)c3c([2H])c([2H])c([2H])c([2H])c3c(-c3ccc4ccccc4c3)c2c1[2H] ; [2H]c1c([2H])c([2H])c2c(-c3cccc4c3oc3c5ccccc5ccc43)c3c([2H])c([2H])c([2H])c([2H])c3c(-c3ccccc3-c3ccccc3)c2c1[2H] ; [2H]c1c([2H])c([2H])c2c(-c3cccc4c3oc3c5ccccc5ccc43)c3c([2H])c([2H])c([2H])c([2H])c3c(-c3cccc(-c4ccccc4)c3)c2c1[2H] ; [2H]c1c([2H])c([2H])c2c(-c3cccc4c3oc3c5ccccc5ccc43)c3c([2H])c([2H])c([2H])c([2H])c3c(-c3ccc(-c4ccccc4)cc3)c2c1[2H]]

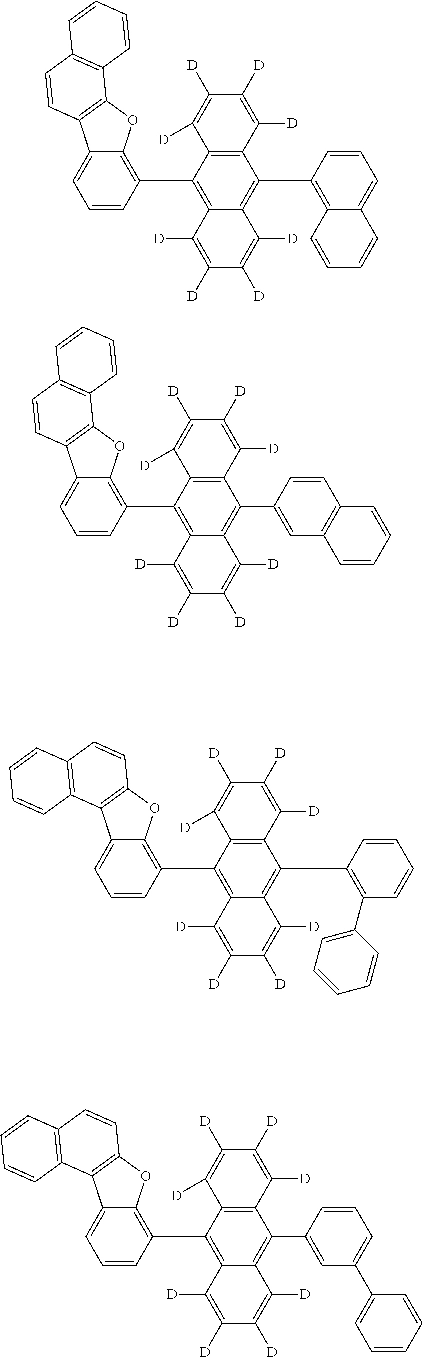 [SMILES: [2H]c1c([2H])c([2H])c2c(-c3cccc4c3oc3c5ccccc5ccc43)c3c([2H])c([2H])c([2H])c([2H])c3c(-c3cccc4ccccc34)c2c1[2H] ; [2H]c1c([2H])c([2H])c2c(-c3cccc4c3oc3c5ccccc5ccc43)c3c([2H])c([2H])c([2H])c([2H])c3c(-c3ccc4ccccc4c3)c2c1[2H] ; [2H]c1c([2H])c([2H])c2c(-c3cccc4c3oc3ccc5ccccc5c34)c3c([2H])c([2H])c([2H])c([2H])c3c(-c3ccccc3-c3ccccc3)c2c1[2H] ; [2H]c1c([2H])c([2H])c2c(-c3cccc4c3oc3ccc5ccccc5c34)c3c([2H])c([2H])c([2H])c([2H])c3c(-c3cccc(-c4ccccc4)c3)c2c1[2H]]

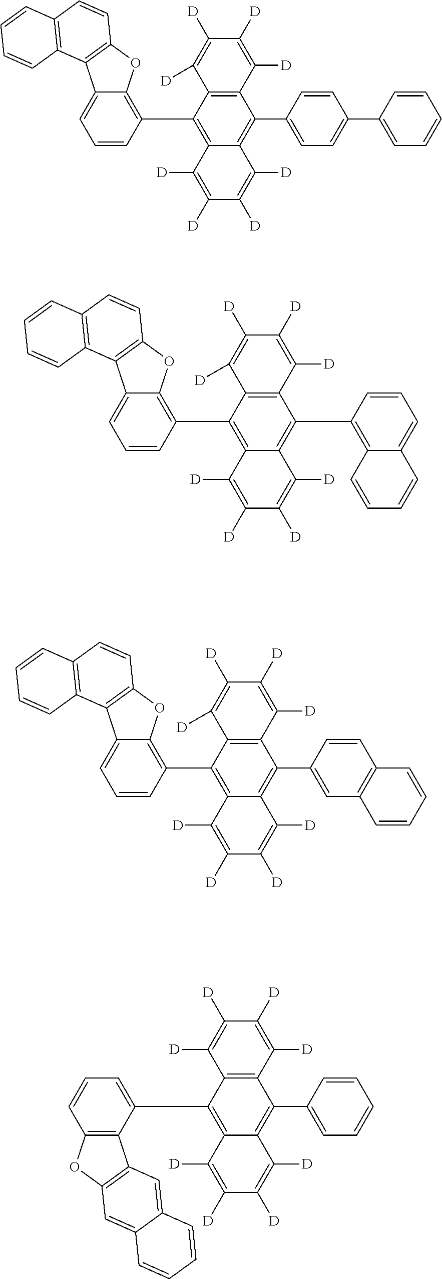 [SMILES: [2H]c1c([2H])c([2H])c2c(-c3cccc4c3oc3ccc5ccccc5c34)c3c([2H])c([2H])c([2H])c([2H])c3c(-c3ccc(-c4ccccc4)cc3)c2c1[2H] ; [2H]c1c([2H])c([2H])c2c(-c3cccc4c3oc3ccc5ccccc5c34)c3c([2H])c([2H])c([2H])c([2H])c3c(-c3cccc4ccccc34)c2c1[2H] ; [2H]c1c([2H])c([2H])c2c(-c3cccc4c3oc3ccc5ccccc5c34)c3c([2H])c([2H])c([2H])c([2H])c3c(-c3ccc4ccccc4c3)c2c1[2H] ; [2H]c1c([2H])c([2H])c2c(-c3cccc4oc5cc6ccccc6cc5c34)c3c([2H])c([2H])c([2H])c([2H])c3c(-c3ccccc3)c2c1[2H]]

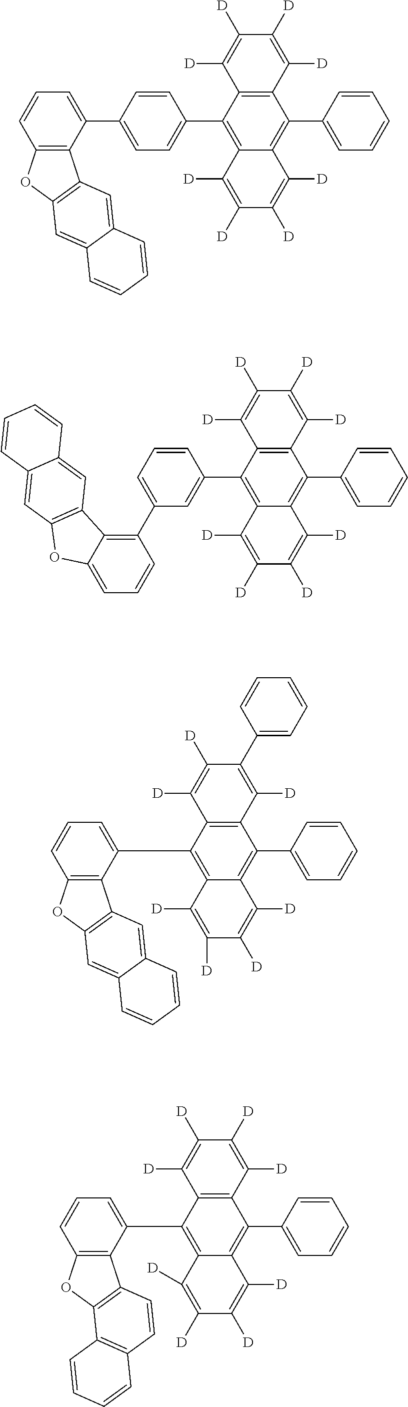 [SMILES: [2H]c1c([2H])c([2H])c2c(-c3ccc(-c4cccc5oc6cc7ccccc7cc6c45)cc3)c3c([2H])c([2H])c([2H])c([2H])c3c(-c3ccccc3)c2c1[2H] ; [2H]c1c([2H])c([2H])c2c(-c3cccc(-c4cccc5oc6cc7ccccc7cc6c45)c3)c3c([2H])c([2H])c([2H])c([2H])c3c(-c3ccccc3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3ccccc3)c2c1[2H]]

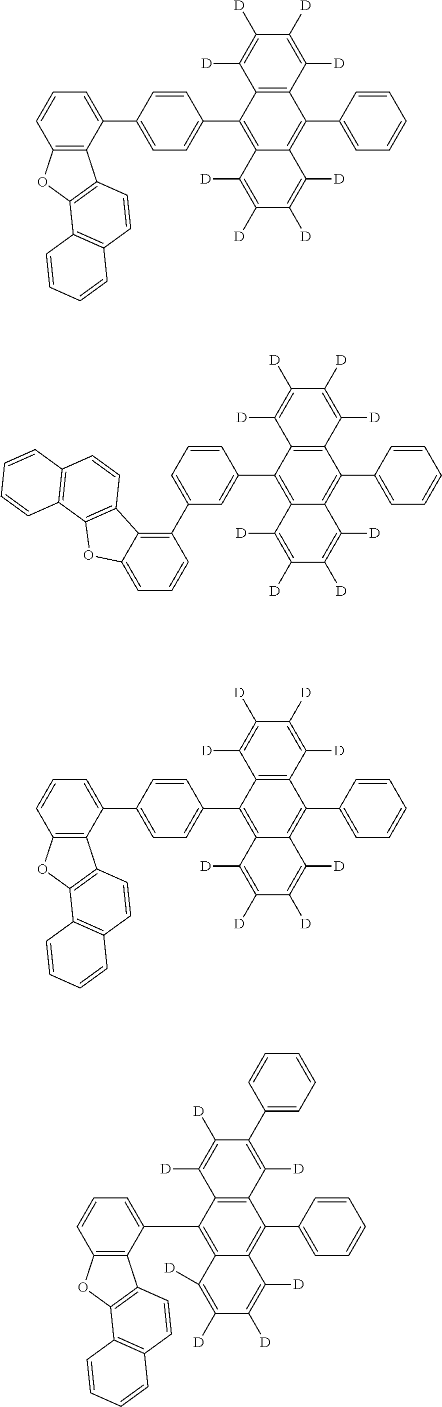 [SMILES: [2H]c1c([2H])c([2H])c2c(-c3ccc(-c4cccc5oc6c7ccccc7ccc6c45)cc3)c3c([2H])c([2H])c([2H])c([2H])c3c(-c3ccccc3)c2c1[2H] ; [2H]c1c([2H])c([2H])c2c(-c3cccc(-c4cccc5oc6c7ccccc7ccc6c45)c3)c3c([2H])c([2H])c([2H])c([2H])c3c(-c3ccccc3)c2c1[2H] ; [2H]c1c([2H])c([2H])c2c(-c3ccc(-c4cccc5oc6c7ccccc7ccc6c45)cc3)c3c([2H])c([2H])c([2H])c([2H])c3c(-c3ccccc3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccccc3)c2c1[2H]]

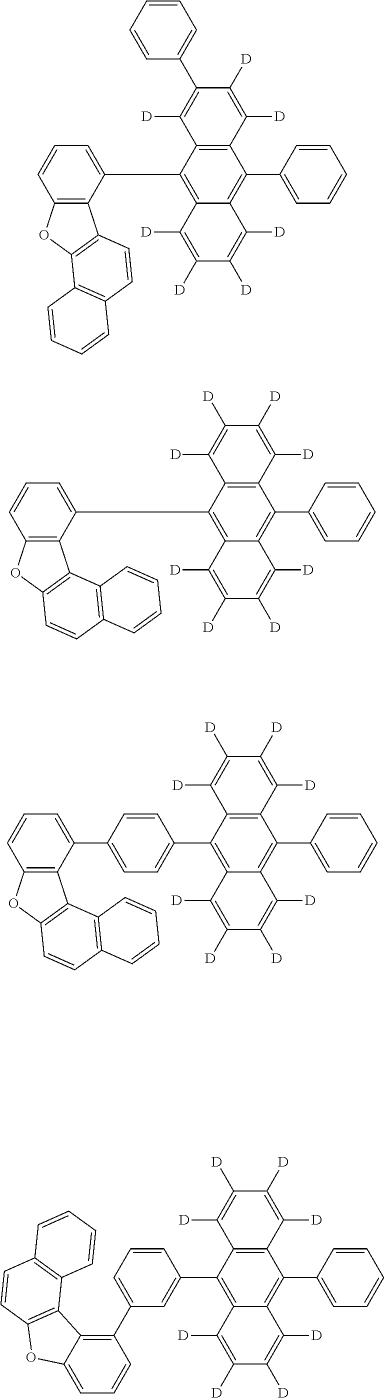 [SMILES: [2H]c1c([2H])c([2H])c2c(-c3cccc4oc5c6ccccc6ccc5c34)c3c([2H])c(-c4ccccc4)c([2H])c([2H])c3c(-c3ccccc3)c2c1[2H] ; [2H]c1c([2H])c([2H])c2c(-c3cccc4oc5ccc6ccccc6c5c34)c3c([2H])c([2H])c([2H])c([2H])c3c(-c3ccccc3)c2c1[2H] ; [2H]c1c([2H])c([2H])c2c(-c3ccc(-c4cccc5oc6ccc7ccccc7c6c45)cc3)c3c([2H])c([2H])c([2H])c([2H])c3c(-c3ccccc3)c2c1[2H] ; [2H]c1c([2H])c([2H])c2c(-c3cccc(-c4cccc5oc6ccc7ccccc7c6c45)c3)c3c([2H])c([2H])c([2H])c([2H])c3c(-c3ccccc3)c2c1[2H]]

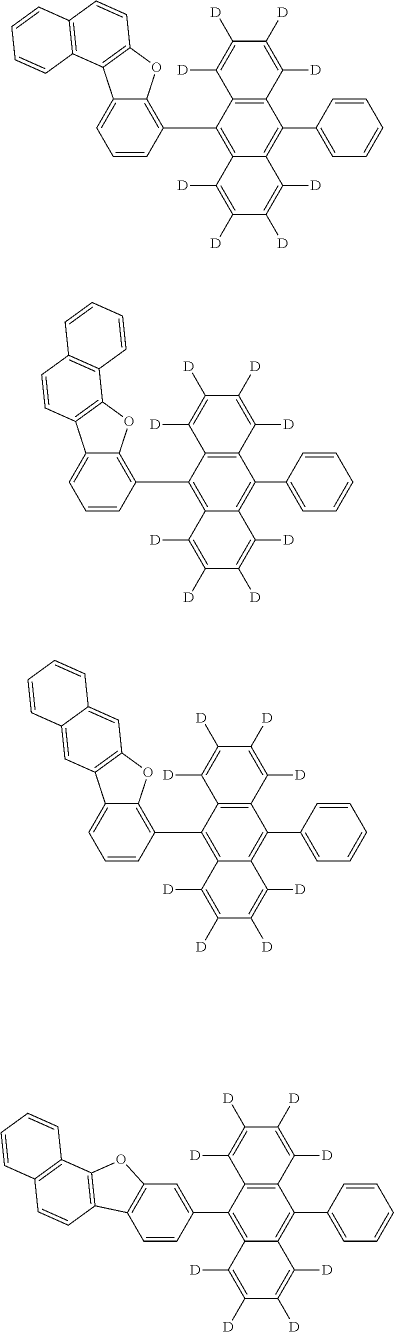 [SMILES: [2H]c1c([2H])c([2H])c2c(-c3cccc4c3oc3ccc5ccccc5c34)c3c([2H])c([2H])c([2H])c([2H])c3c(-c3ccccc3)c2c1[2H] ; [2H]c1c([2H])c([2H])c2c(-c3cccc4c3oc3c5ccccc5ccc43)c3c([2H])c([2H])c([2H])c([2H])c3c(-c3ccccc3)c2c1[2H] ; [2H]c1c([2H])c([2H])c2c(-c3cccc4c3oc3cc5ccccc5cc34)c3c([2H])c([2H])c([2H])c([2H])c3c(-c3ccccc3)c2c1[2H] ; [2H]c1c([2H])c([2H])c2c(-c3ccc4c(c3)oc3c5ccccc5ccc43)c3c([2H])c([2H])c([2H])c([2H])c3c(-c3ccccc3)c2c1[2H]]

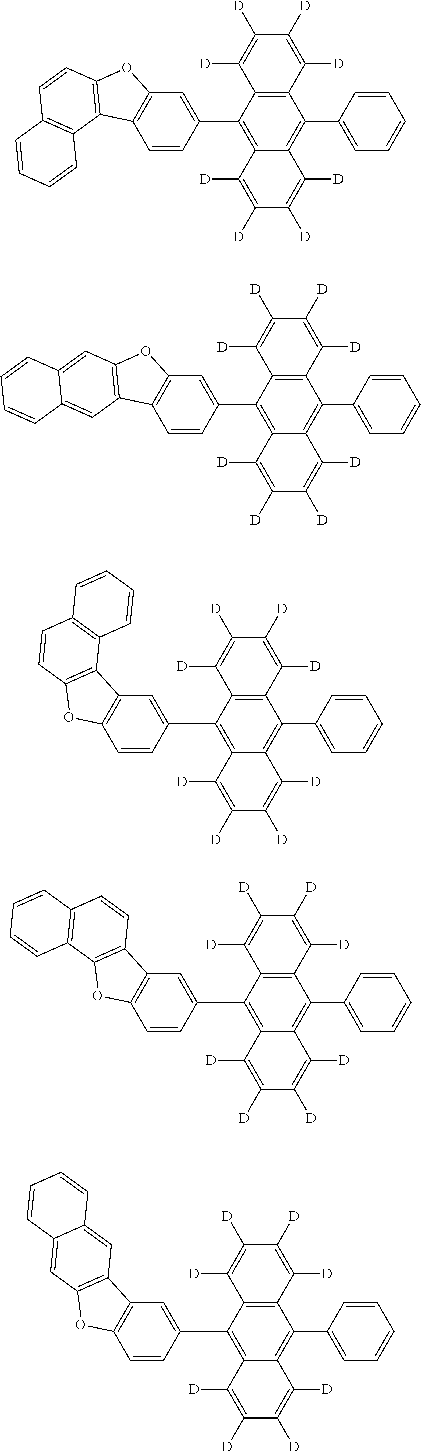

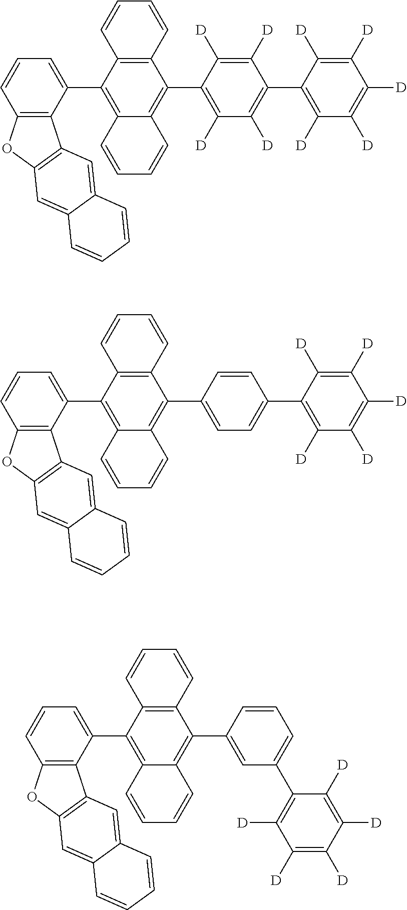 [SMILES: [2H]c1c([2H])c([2H])c(-c2c([2H])c([2H])c(-c3c4ccccc4c(-c4cccc5oc6cc7ccccc7cc6c45)c4ccccc34)c([2H])c2[2H])c([2H])c1[2H] ; [2H]c1c([2H])c([2H])c(-c2ccc(-c3c4ccccc4c(-c4cccc5oc6cc7ccccc7cc6c45)c4ccccc34)cc2)c([2H])c1[2H] ; [2H]c1c([2H])c([2H])c(-c2cccc(-c3c4ccccc4c(-c4cccc5oc6cc7ccccc7cc6c45)c4ccccc34)c2)c([2H])c1[2H]]

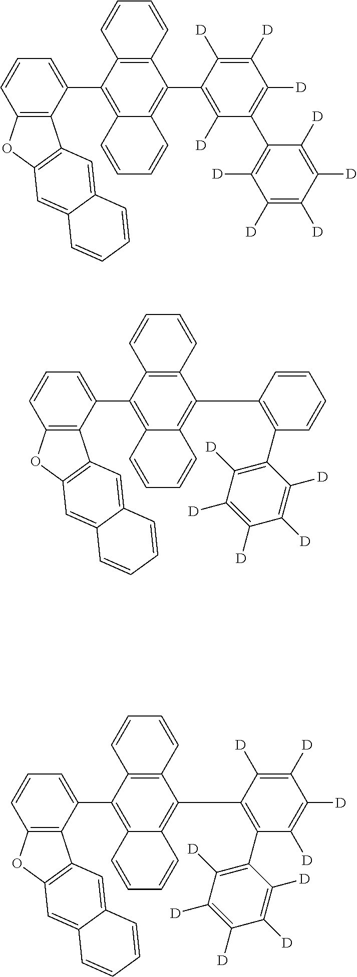 [SMILES: [2H]c1c([2H])c([2H])c(-c2c([2H])c([2H])c([2H])c(-c3c4ccccc4c(-c4cccc5oc6cc7ccccc7cc6c45)c4ccccc34)c2[2H])c([2H])c1[2H] ; [2H]c1c([2H])c([2H])c(-c2ccccc2-c2c3ccccc3c(-c3cccc4oc5cc6ccccc6cc5c34)c3ccccc23)c([2H])c1[2H] ; [2H]c1c([2H])c([2H])c(-c2c([2H])c([2H])c([2H])c([2H])c2-c2c3ccccc3c(-c3cccc4oc5cc6ccccc6cc5c34)c3ccccc23)c([2H])c1[2H]]

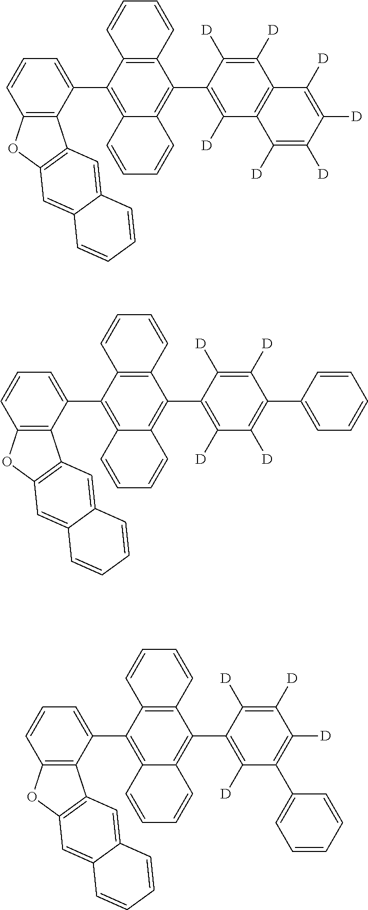 [SMILES: [2H]c1c([2H])c([2H])c2c([2H])c(-c3c4ccccc4c(-c4cccc5oc6cc7ccccc7cc6c45)c4ccccc34)c([2H])c([2H])c2c1[2H] ; [2H]c1c([2H])c(-c2c3ccccc3c(-c3cccc4oc5cc6ccccc6cc5c34)c3ccccc23)c([2H])c([2H])c1-c1ccccc1 ; [2H]c1c([2H])c(-c2ccccc2)c([2H])c(-c2c3ccccc3c(-c3cccc4oc5cc6ccccc6cc5c34)c3ccccc23)c1[2H]]

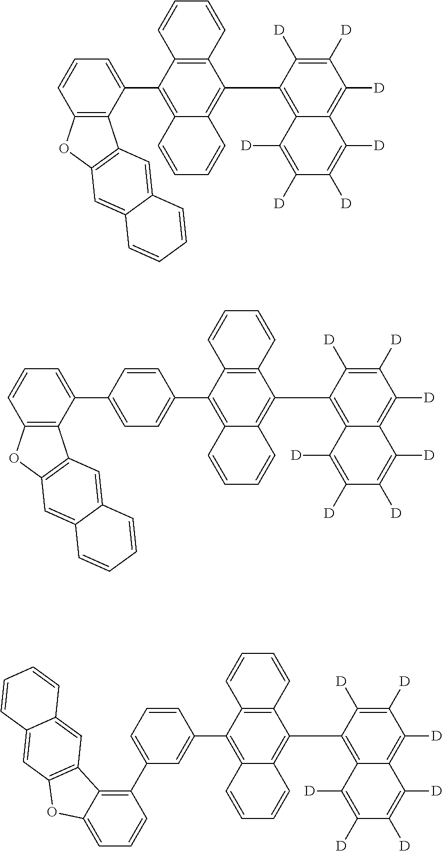 [SMILES: [2H]c1c([2H])c([2H])c2c(-c3c4ccccc4c(-c4cccc5oc6cc7ccccc7cc6c45)c4ccccc34)c([2H])c([2H])c([2H])c2c1[2H] ; [2H]c1c([2H])c([2H])c2c(-c3c4ccccc4c(-c4ccc(-c5cccc6oc7cc8ccccc8cc7c56)cc4)c4ccccc34)c([2H])c([2H])c([2H])c2c1[2H] ; [2H]c1c([2H])c([2H])c2c(-c3c4ccccc4c(-c4cccc(-c5cccc6oc7cc8ccccc8cc7c56)c4)c4ccccc34)c([2H])c([2H])c([2H])c2c1[2H]]

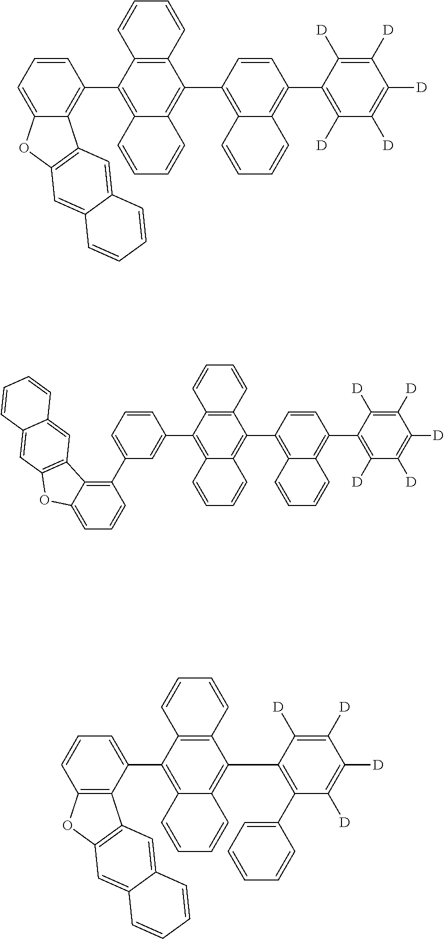 [SMILES: [2H]c1c([2H])c([2H])c(-c2ccc(-c3c4ccccc4c(-c4cccc5oc6cc7ccccc7cc6c45)c4ccccc34)c3ccccc23)c([2H])c1[2H] ; [2H]c1c([2H])c([2H])c(-c2ccc(-c3c4ccccc4c(-c4cccc(-c5cccc6oc7cc8ccccc8cc7c56)c4)c4ccccc34)c3ccccc23)c([2H])c1[2H] ; [2H]c1c([2H])c([2H])c(-c2c3ccccc3c(-c3cccc4oc5cc6ccccc6cc5c34)c3ccccc23)c(-c2ccccc2)c1[2H]]

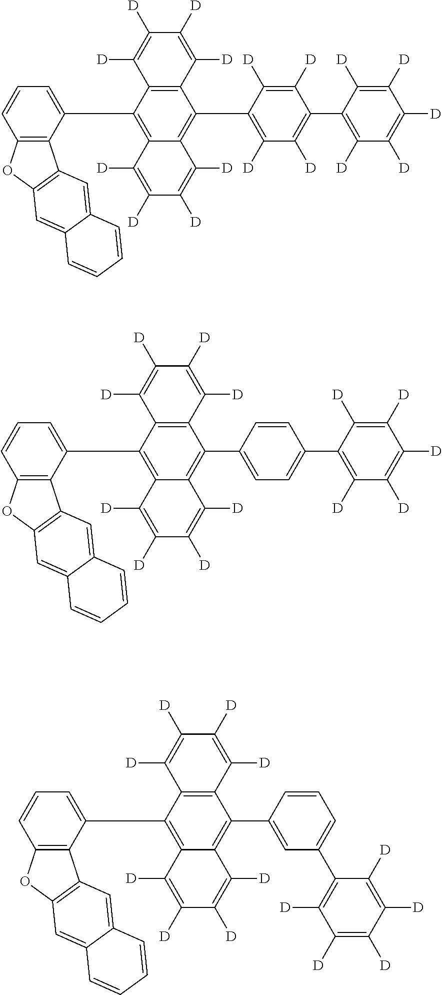 [SMILES: [2H]c1c([2H])c([2H])c(-c2c([2H])c([2H])c(-c3c4c([2H])c([2H])c([2H])c([2H])c4c(-c4cccc5oc6cc7ccccc7cc6c45)c4c([2H])c([2H])c([2H])c([2H])c34)c([2H])c2[2H])c([2H])c1[2H] ; [2H]c1c([2H])c([2H])c(-c2ccc(-c3c4c([2H])c([2H])c([2H])c([2H])c4c(-c4cccc5oc6cc7ccccc7cc6c45)c4c([2H])c([2H])c([2H])c([2H])c34)cc2)c([2H])c1[2H] ; [2H]c1c([2H])c([2H])c(-c2cccc(-c3c4c([2H])c([2H])c([2H])c([2H])c4c(-c4cccc5oc6cc7ccccc7cc6c45)c4c([2H])c([2H])c([2H])c([2H])c34)c2)c([2H])c1[2H]]

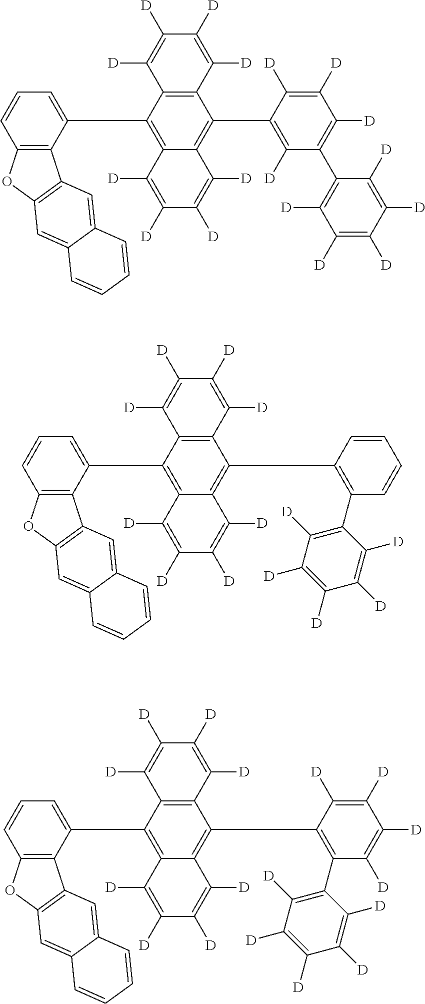 [SMILES: [2H]c1c([2H])c([2H])c(-c2c([2H])c([2H])c([2H])c(-c3c4c([2H])c([2H])c([2H])c([2H])c4c(-c4cccc5oc6cc7ccccc7cc6c45)c4c([2H])c([2H])c([2H])c([2H])c34)c2[2H])c([2H])c1[2H] ; [2H]c1c([2H])c([2H])c(-c2ccccc2-c2c3c([2H])c([2H])c([2H])c([2H])c3c(-c3cccc4oc5cc6ccccc6cc5c34)c3c([2H])c([2H])c([2H])c([2H])c23)c([2H])c1[2H] ; [2H]c1c([2H])c([2H])c(-c2c([2H])c([2H])c([2H])c([2H])c2-c2c3c([2H])c([2H])c([2H])c([2H])c3c(-c3cccc4oc5cc6ccccc6cc5c34)c3c([2H])c([2H])c([2H])c([2H])c23)c([2H])c1[2H]]

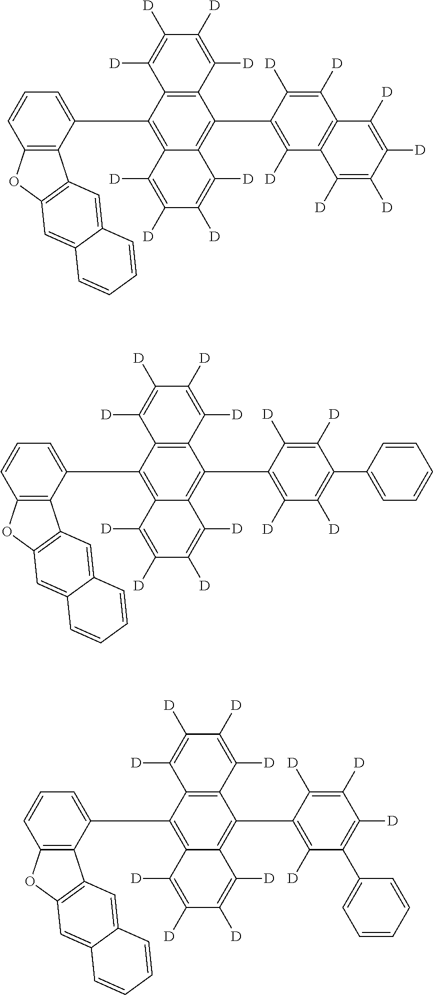 [SMILES: [2H]c1c([2H])c([2H])c2c([2H])c(-c3c4c([2H])c([2H])c([2H])c([2H])c4c(-c4cccc5oc6cc7ccccc7cc6c45)c4c([2H])c([2H])c([2H])c([2H])c34)c([2H])c([2H])c2c1[2H] ; [2H]c1c([2H])c(-c2c3c([2H])c([2H])c([2H])c([2H])c3c(-c3cccc4oc5cc6ccccc6cc5c34)c3c([2H])c([2H])c([2H])c([2H])c23)c([2H])c([2H])c1-c1ccccc1 ; [2H]c1c([2H])c(-c2ccccc2)c([2H])c(-c2c3c([2H])c([2H])c([2H])c([2H])c3c(-c3cccc4oc5cc6ccccc6cc5c34)c3c([2H])c([2H])c([2H])c([2H])c23)c1[2H]]

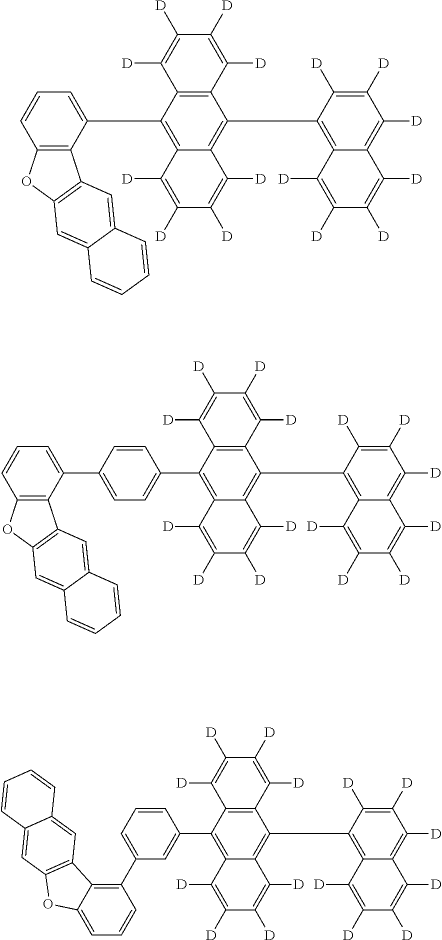 [SMILES: [2H]c1c([2H])c([2H])c2c(-c3c4c([2H])c([2H])c([2H])c([2H])c4c(-c4cccc5oc6cc7ccccc7cc6c45)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4ccc(-c5cccc6oc7cc8ccccc8cc7c56)cc4)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4cccc(-c5cccc6oc7cc8ccccc8cc7c56)c4)c4c([2H])c([2H])c([2H])c([2H])c34)c([2H])c([2H])c([2H])c2c1[2H]]

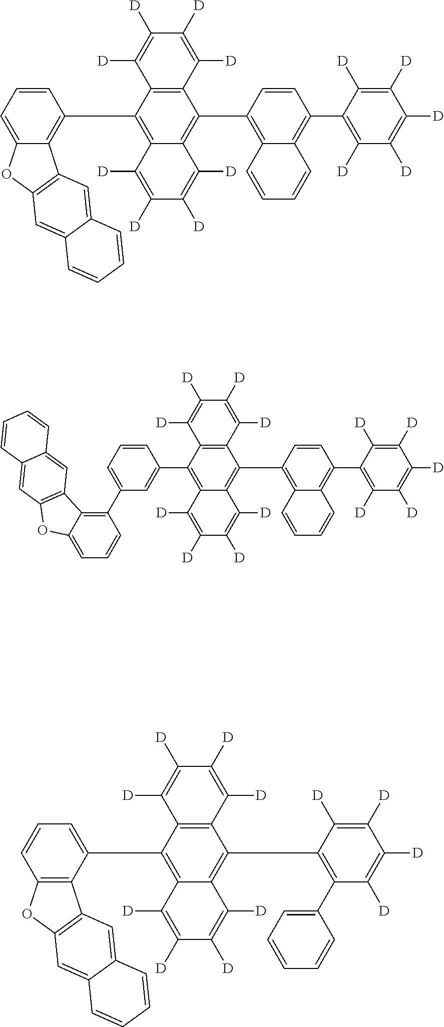 [SMILES: [2H]c1c([2H])c([2H])c(-c2ccc(-c3c4c([2H])c([2H])c([2H])c([2H])c4c(-c4cccc5oc6cc7ccccc7cc6c45)c4c([2H])c([2H])c([2H])c([2H])c34)c3ccccc23)c([2H])c1[2H] ; [2H]c1c([2H])c([2H])c(-c2ccc(-c3c4c([2H])c([2H])c([2H])c([2H])c4c(-c4cccc(-c5cccc6oc7cc8ccccc8cc7c56)c4)c4c([2H])c([2H])c([2H])c([2H])c34)c3ccccc23)c([2H])c1[2H] ; [2H]c1c([2H])c([2H])c(-c2c3c([2H])c([2H])c([2H])c([2H])c3c(-c3cccc4oc5cc6ccccc6cc5c34)c3c([2H])c([2H])c([2H])c([2H])c23)c(-c2ccccc2)c1[2H]]

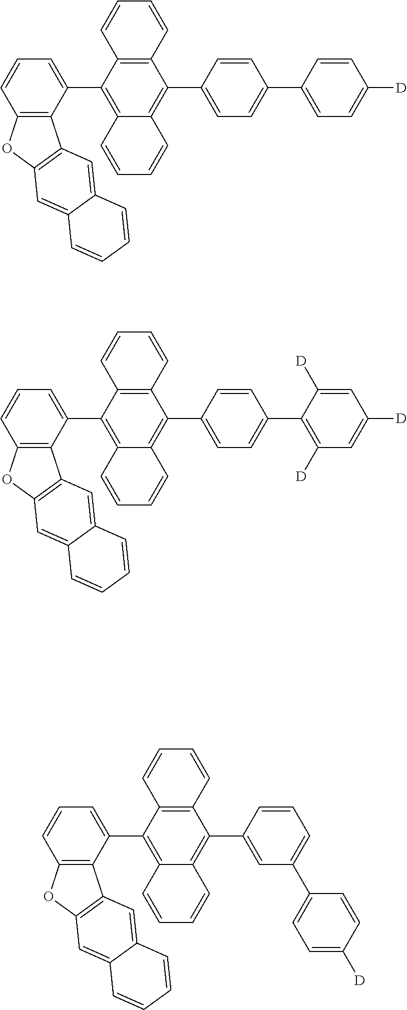 [SMILES: [2H]c1ccc(-c2ccc(-c3c4ccccc4c(-c4cccc5oc6cc7ccccc7cc6c45)c4ccccc34)cc2)cc1 ; [2H]c1cc([2H])c(-c2ccc(-c3c4ccccc4c(-c4cccc5oc6cc7ccccc7cc6c45)c4ccccc34)cc2)c([2H])c1 ; [2H]c1ccc(-c2cccc(-c3c4ccccc4c(-c4cccc5oc6cc7ccccc7cc6c45)c4ccccc34)c2)cc1]

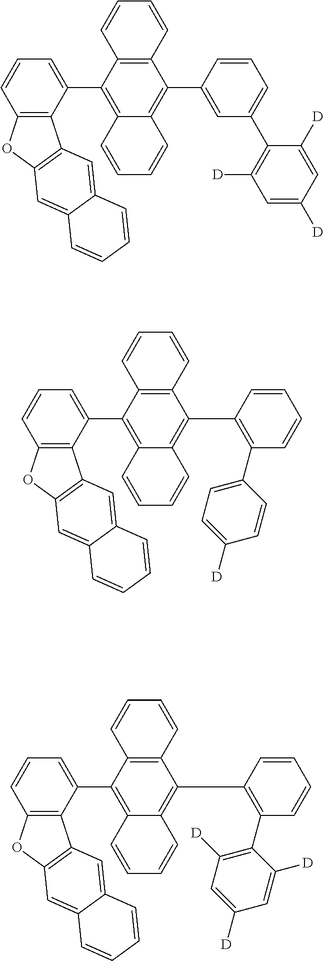 [SMILES: [2H]c1cc([2H])c(-c2cccc(-c3c4ccccc4c(-c4cccc5oc6cc7ccccc7cc6c45)c4ccccc34)c2)c([2H])c1 ; [2H]c1ccc(-c2ccccc2-c2c3ccccc3c(-c3cccc4oc5cc6ccccc6cc5c34)c3ccccc23)cc1 ; [2H]c1cc([2H])c(-c2ccccc2-c2c3ccccc3c(-c3cccc4oc5cc6ccccc6cc5c34)c3ccccc23)c([2H])c1]

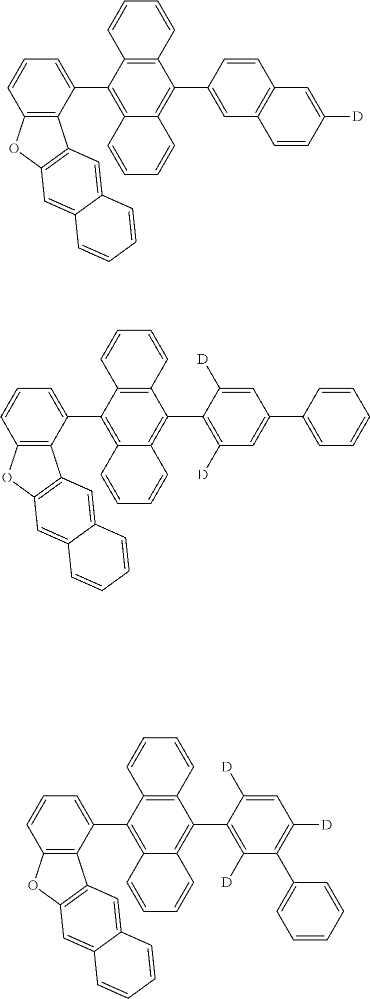 [SMILES: [2H]c1ccc2cc(-c3c4ccccc4c(-c4cccc5oc6cc7ccccc7cc6c45)c4ccccc34)ccc2c1 ; [2H]c1cc(-c2ccccc2)cc([2H])c1-c1c2ccccc2c(-c2cccc3oc4cc5ccccc5cc4c23)c2ccccc12 ; [2H]c1cc([2H])c(-c2c3ccccc3c(-c3cccc4oc5cc6ccccc6cc5c34)c3ccccc23)c([2H])c1-c1ccccc1]

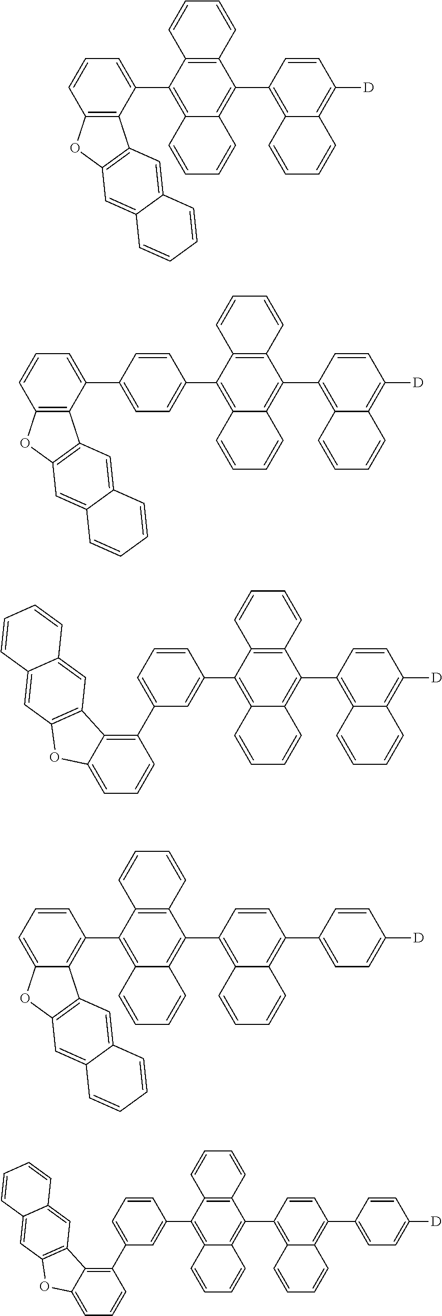 [SMILES: [2H]c1ccc(-c2c3ccccc3c(-c3cccc4oc5cc6ccccc6cc5c34)c3ccccc23)c2ccccc12 ; [2H]c1ccc(-c2c3ccccc3c(-c3ccc(-c4cccc5oc6cc7ccccc7cc6c45)cc3)c3ccccc23)c2ccccc12 ; [2H]c1ccc(-c2c3ccccc3c(-c3cccc(-c4cccc5oc6cc7ccccc7cc6c45)c3)c3ccccc23)c2ccccc12 ; [2H]c1ccc(-c2ccc(-c3c4ccccc4c(-c4cccc5oc6cc7ccccc7cc6c45)c4ccccc34)c3ccccc23)cc1 ; [2H]c1ccc(-c2ccc(-c3c4ccccc4c(-c4cccc(-c5cccc6oc7cc8ccccc8cc7c56)c4)c4ccccc34)c3ccccc23)cc1]

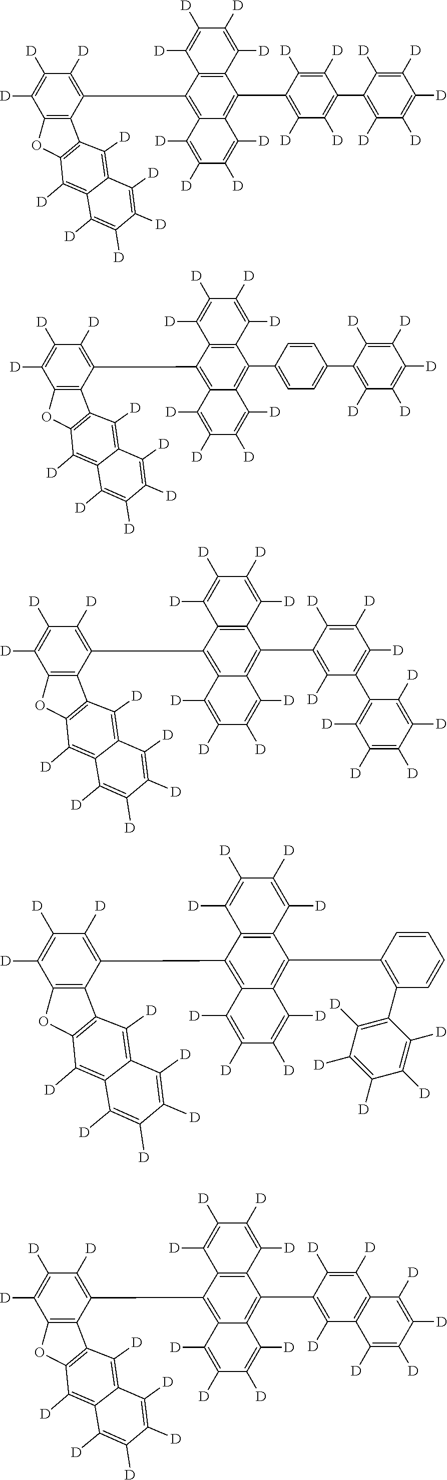

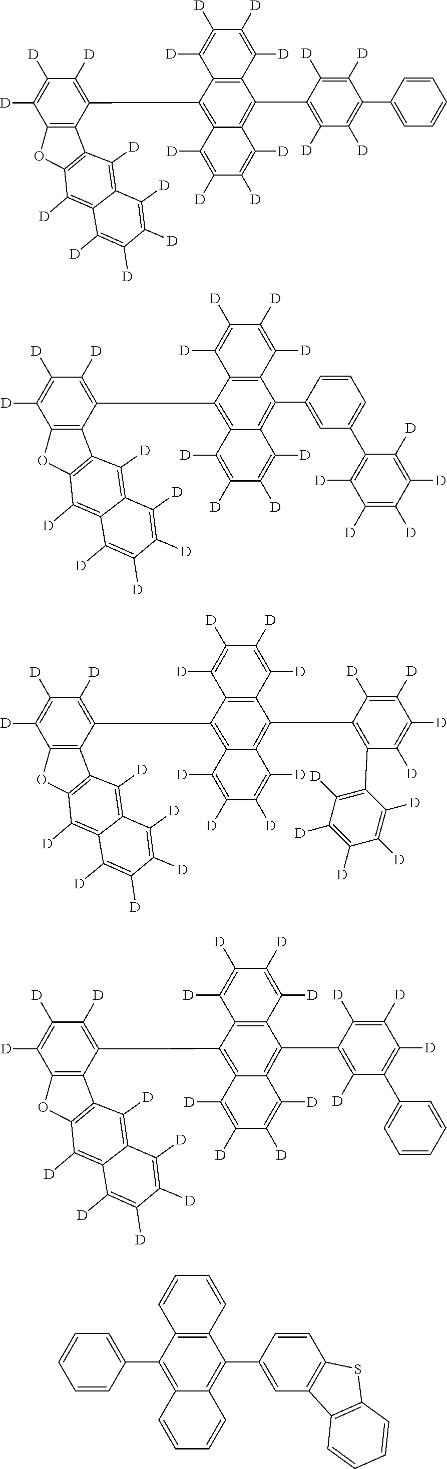

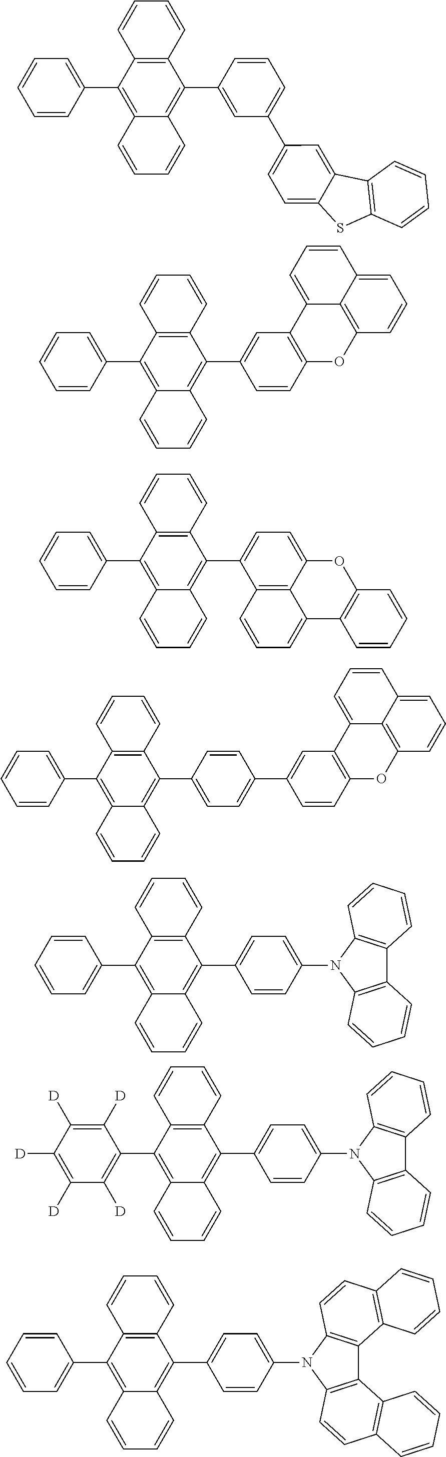

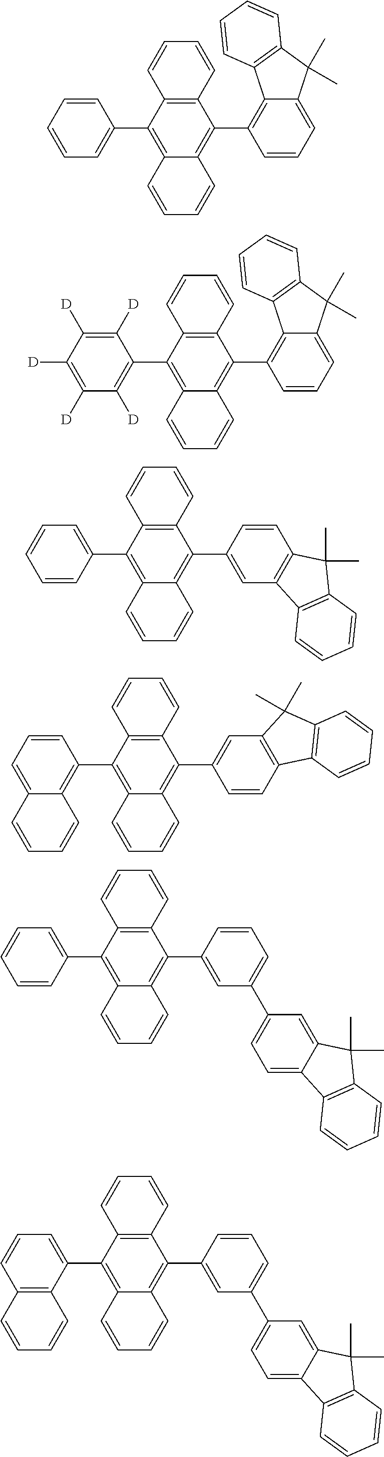

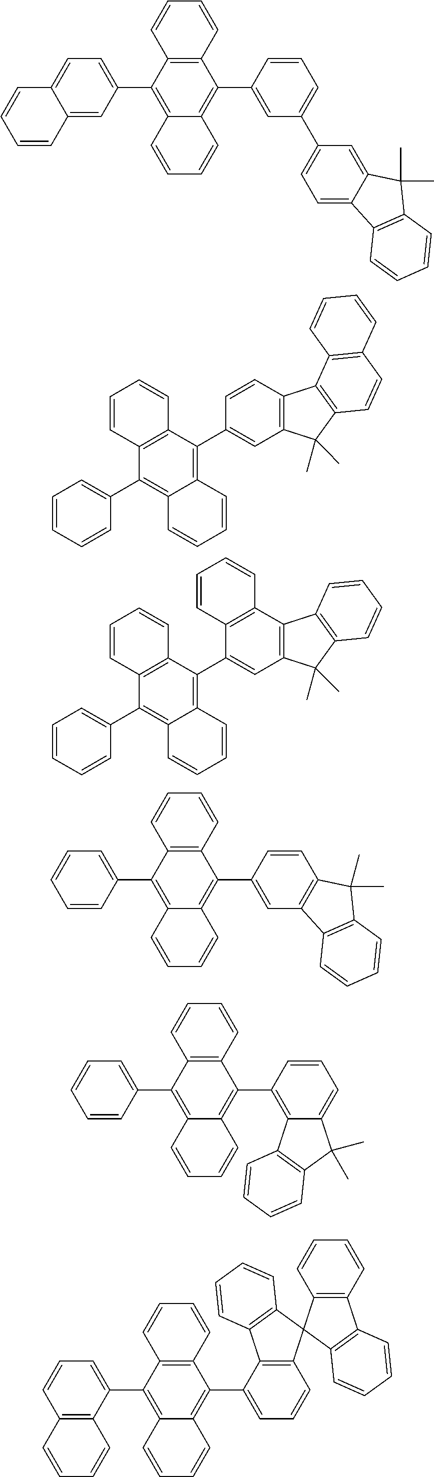

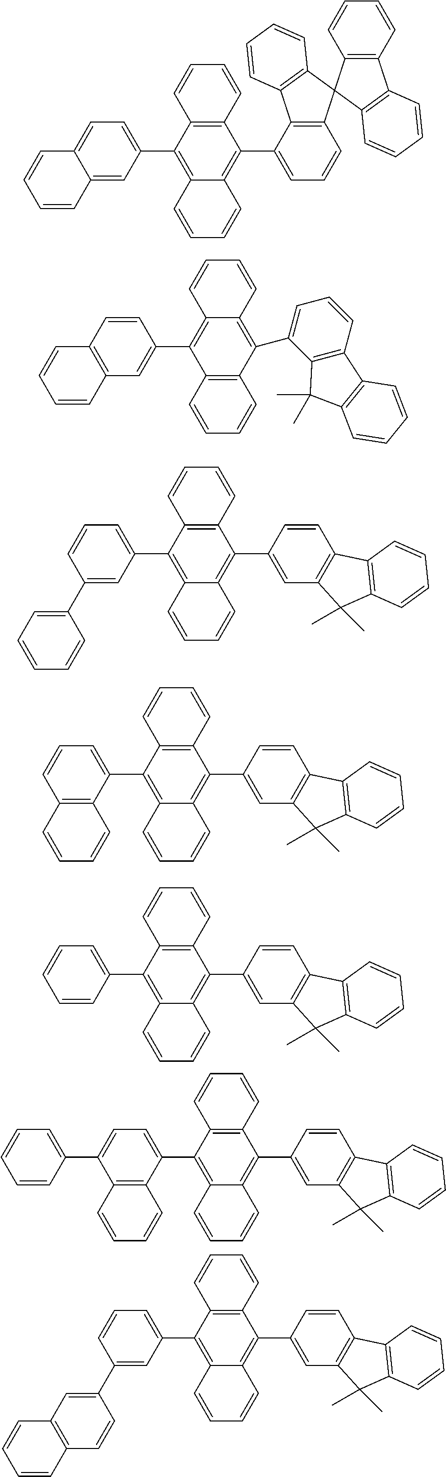

In the following specific examples of the compounds, D represents a deuterium atom, z, z1, z4, z5 and z6 each represent the number of the deuterium atom bonded to a ring, z is an integer of 1 to 8, z1 is an integer of 1 to 9, z4 to z5 are each an integer of 1 to 5, and z6 is an integer of 1 to 7.

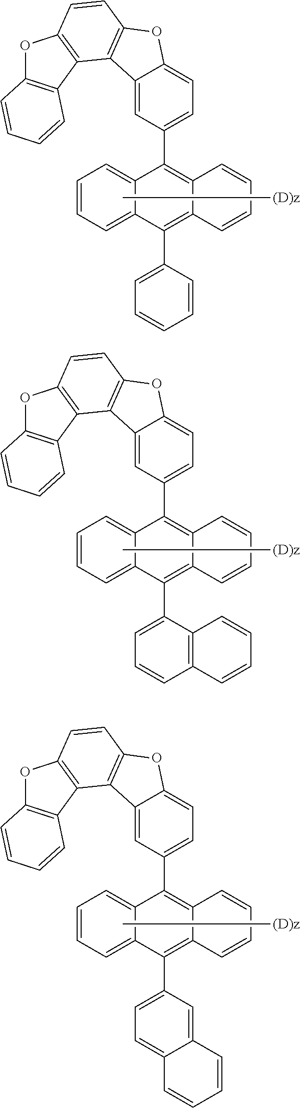

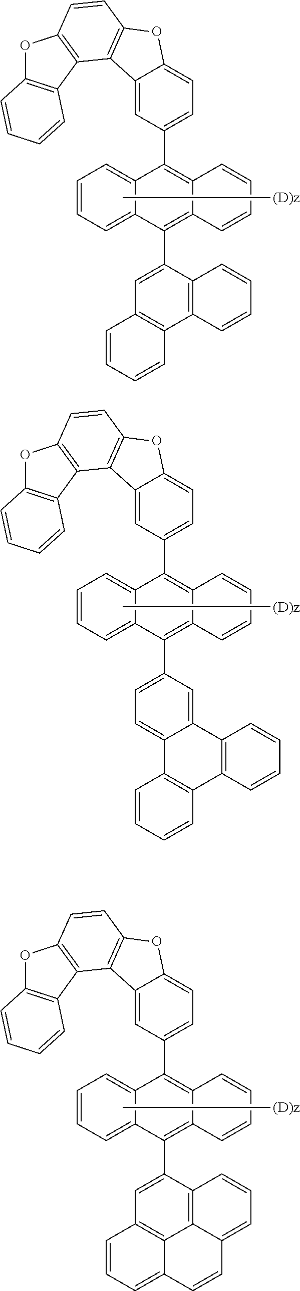

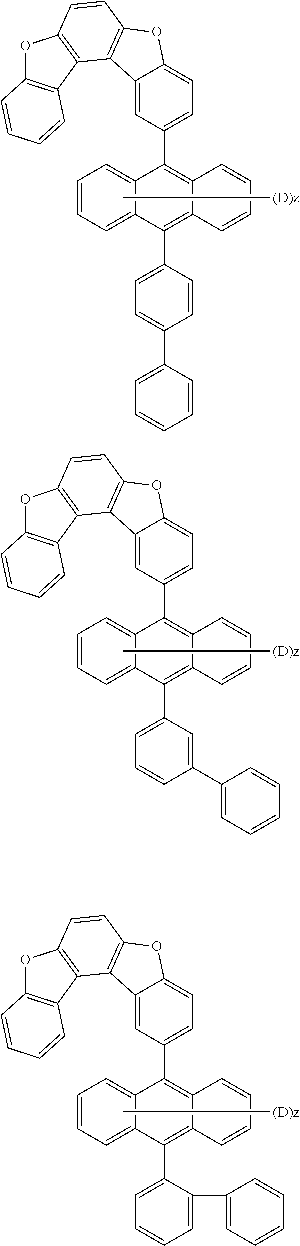

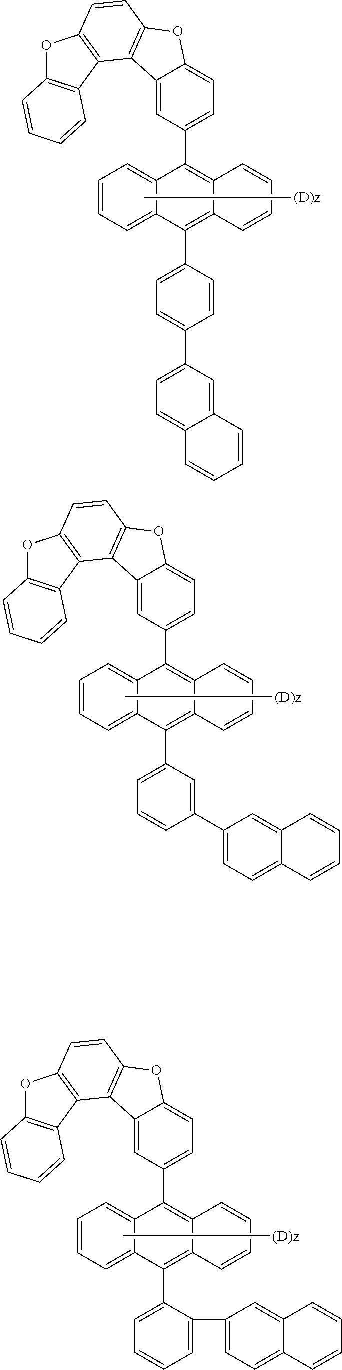

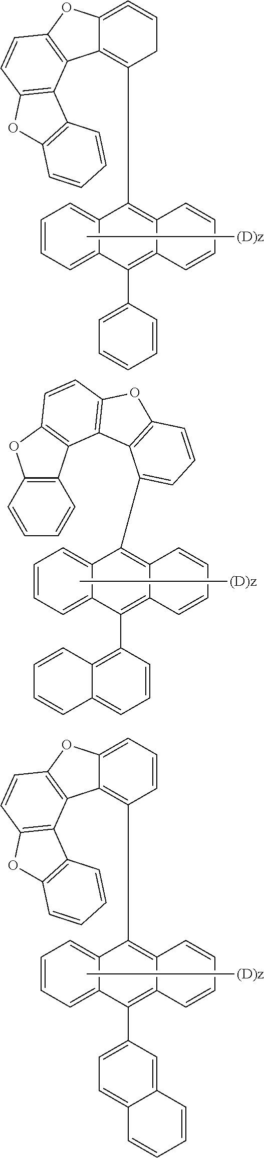

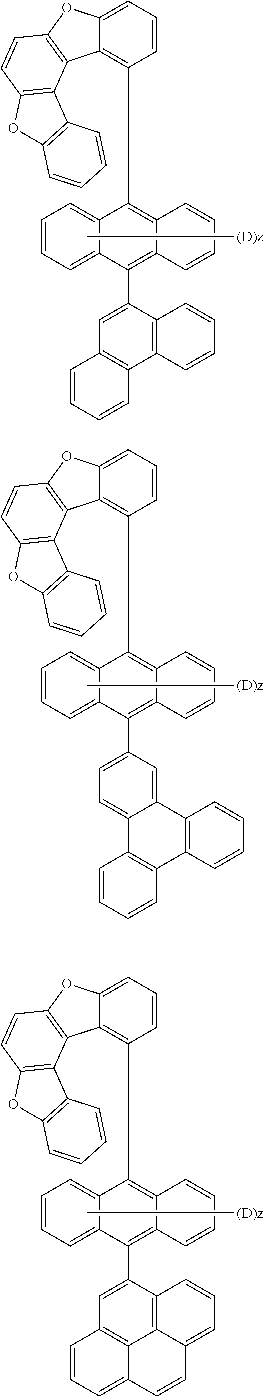

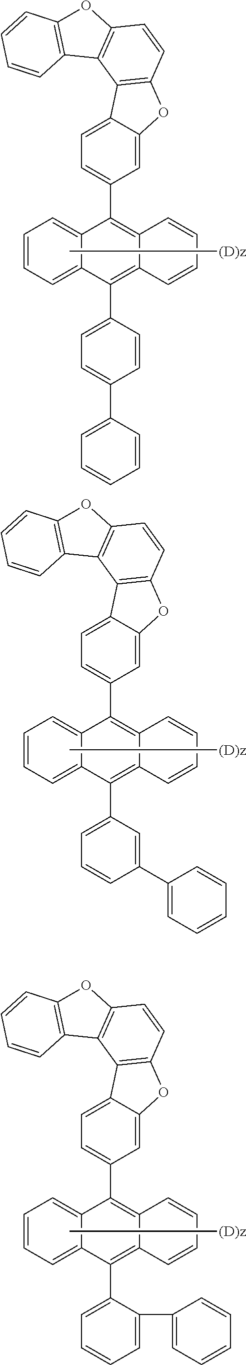

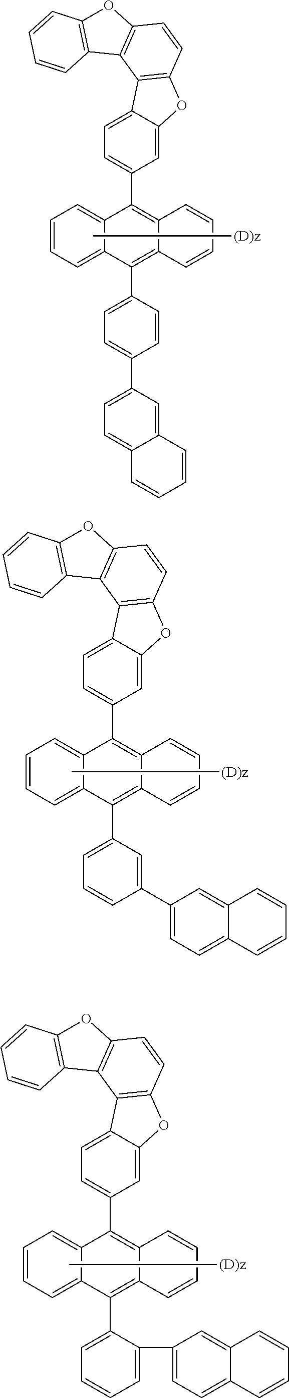

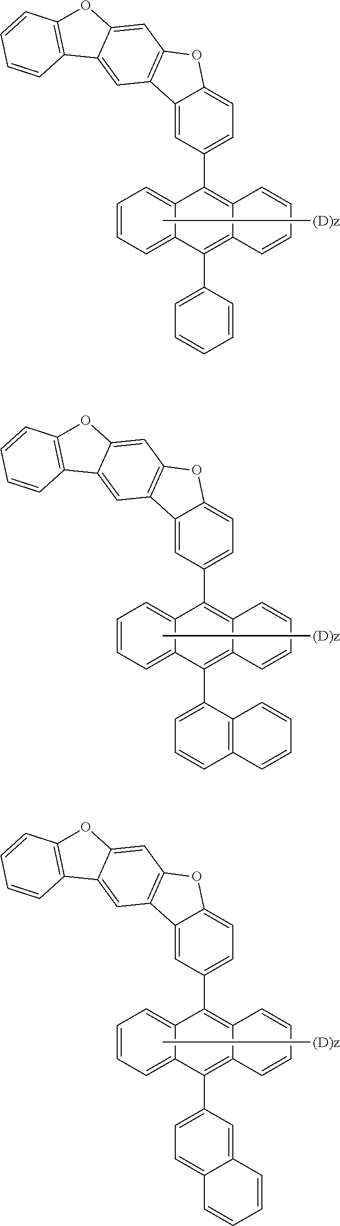

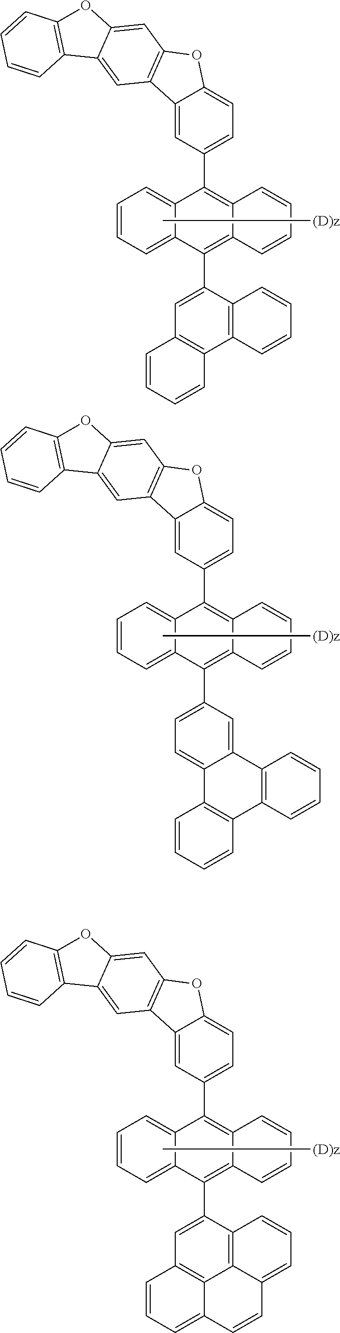

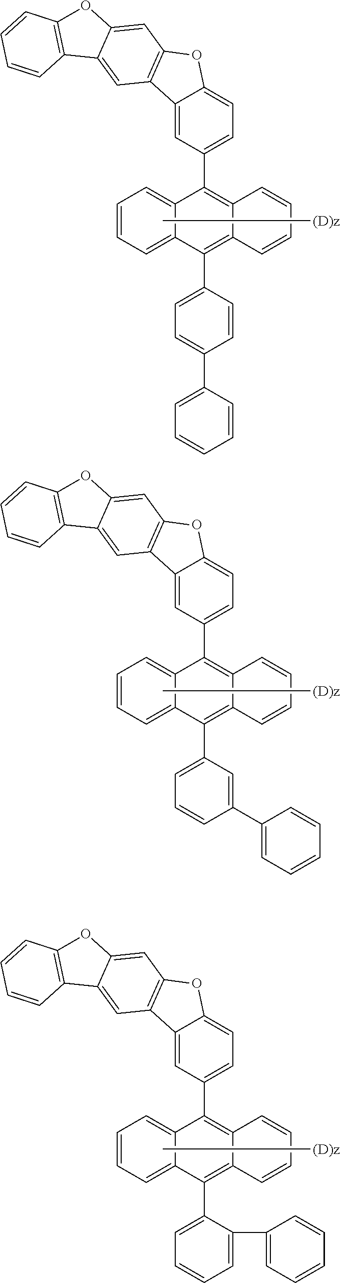

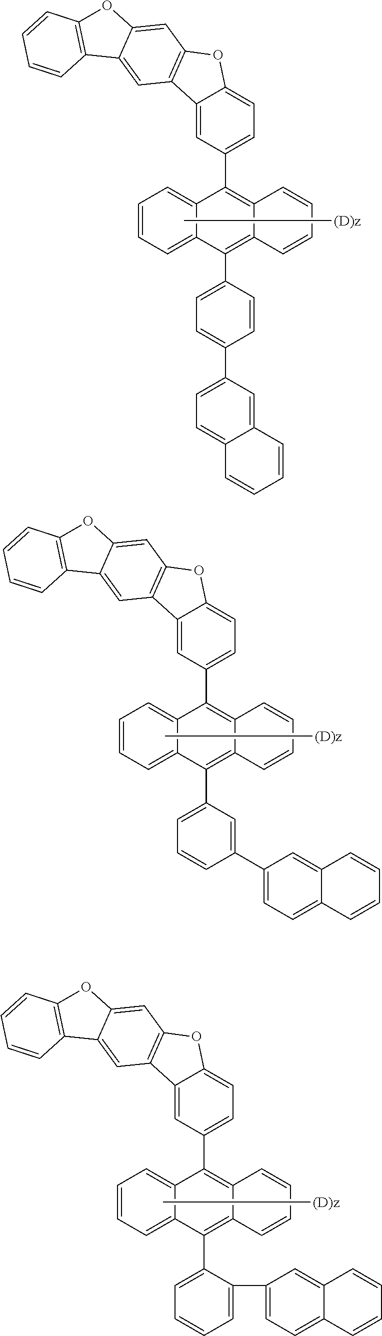

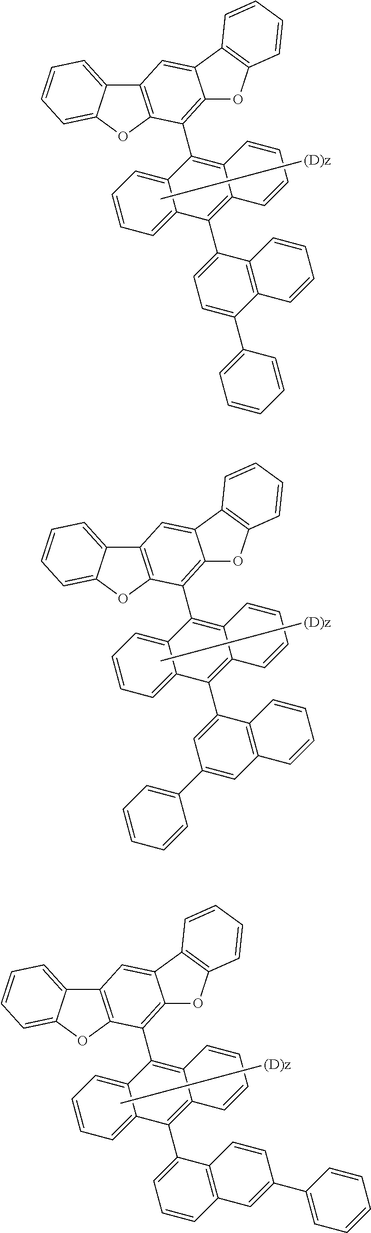

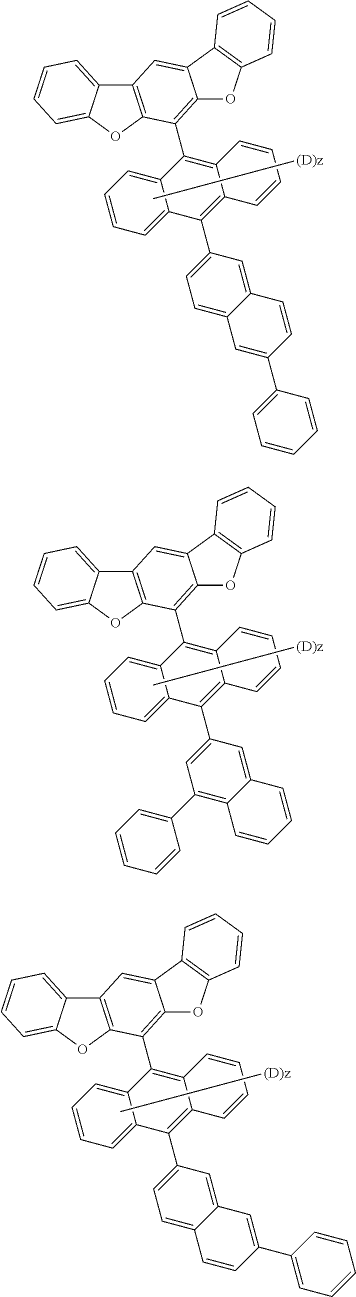

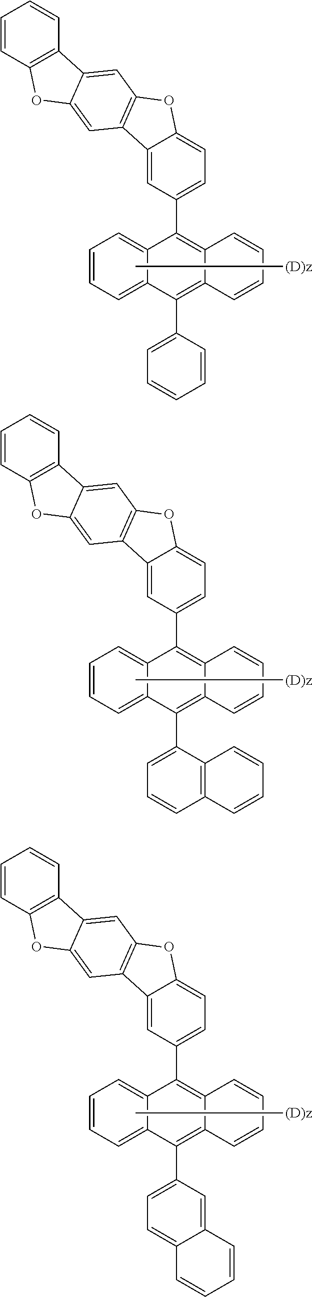

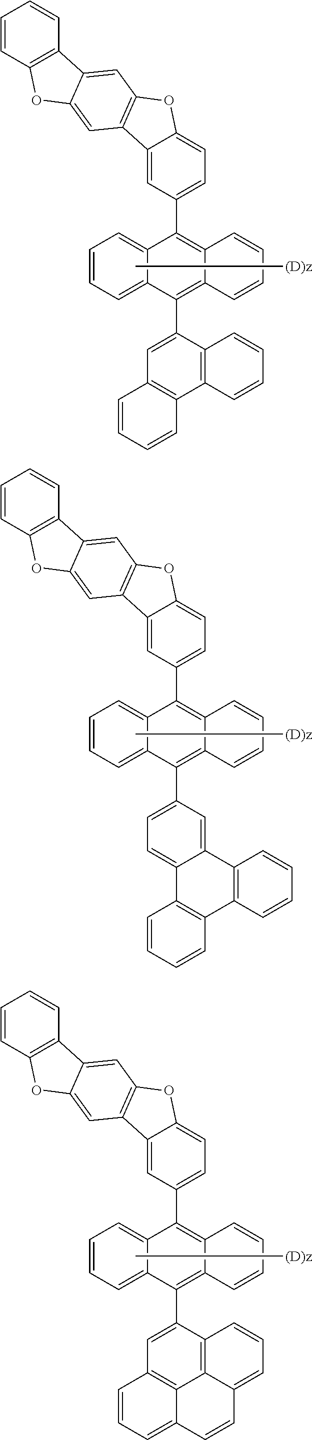

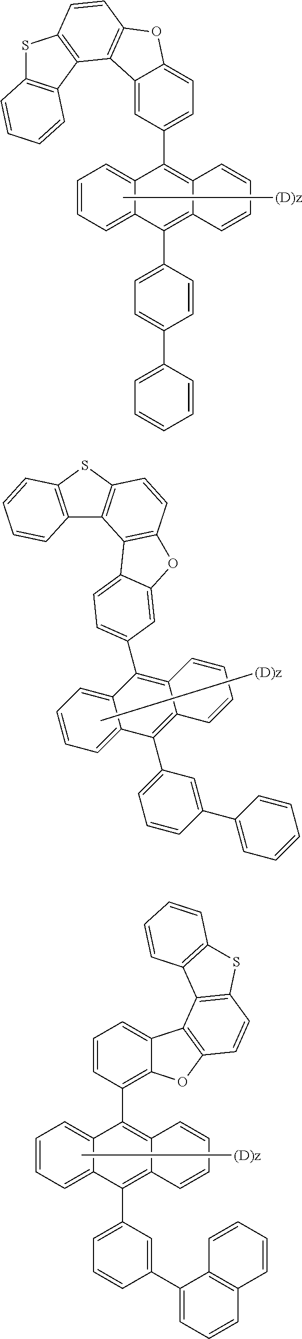

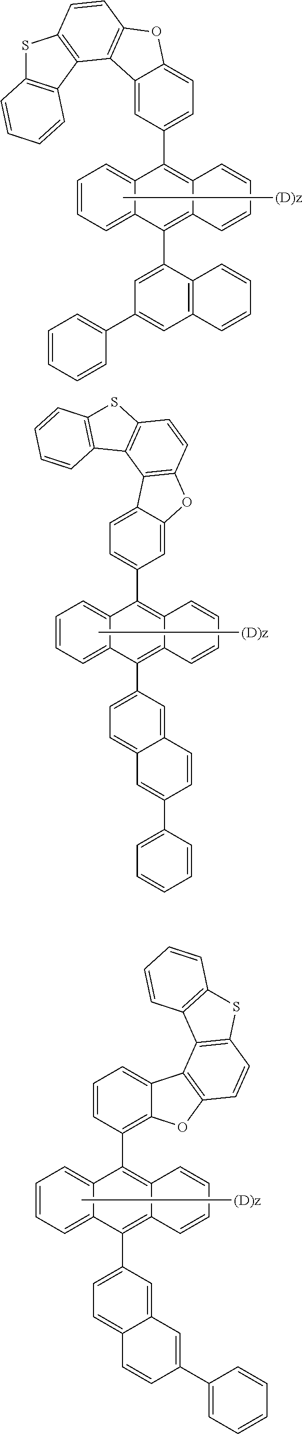

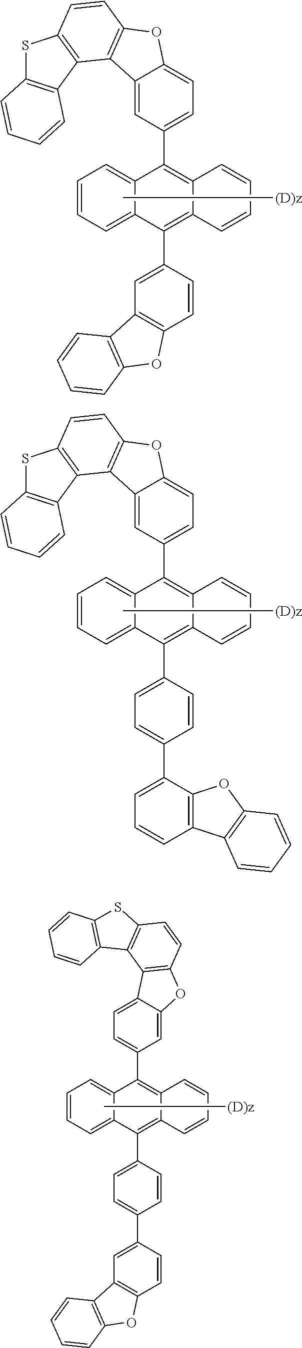

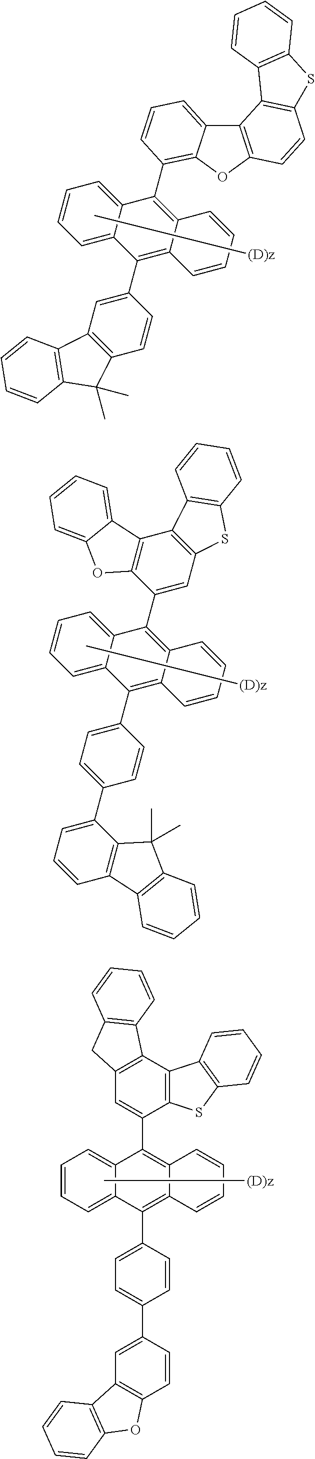

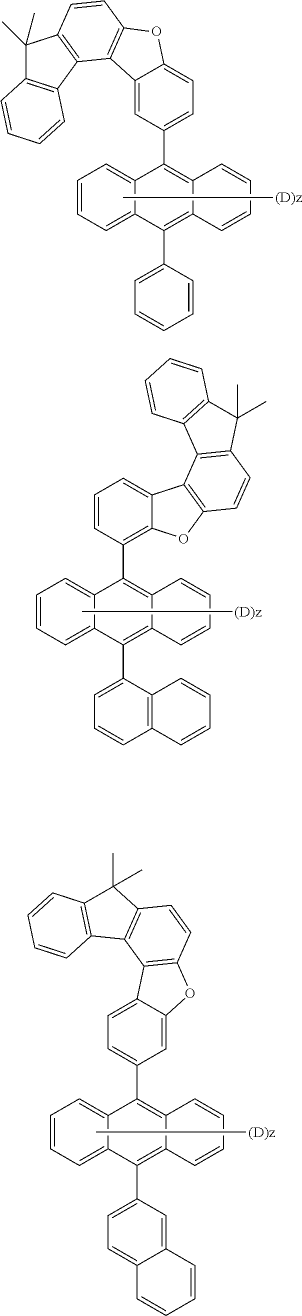

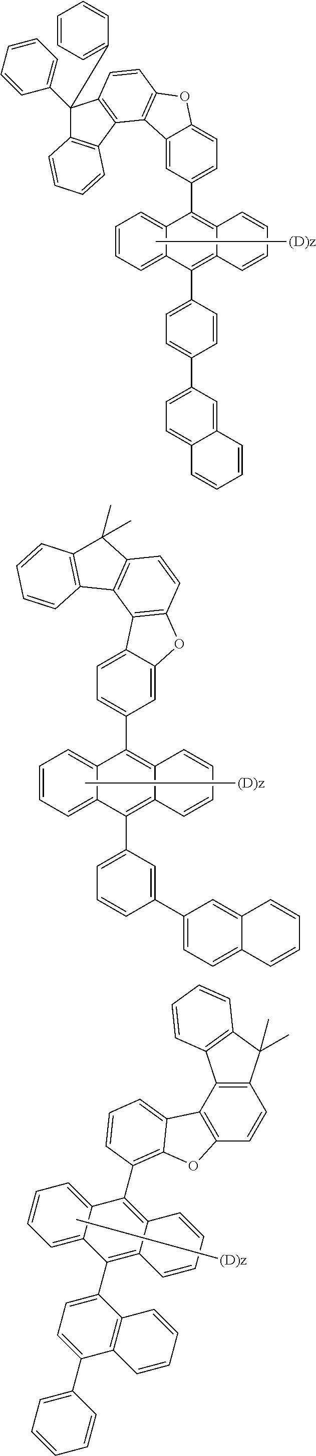

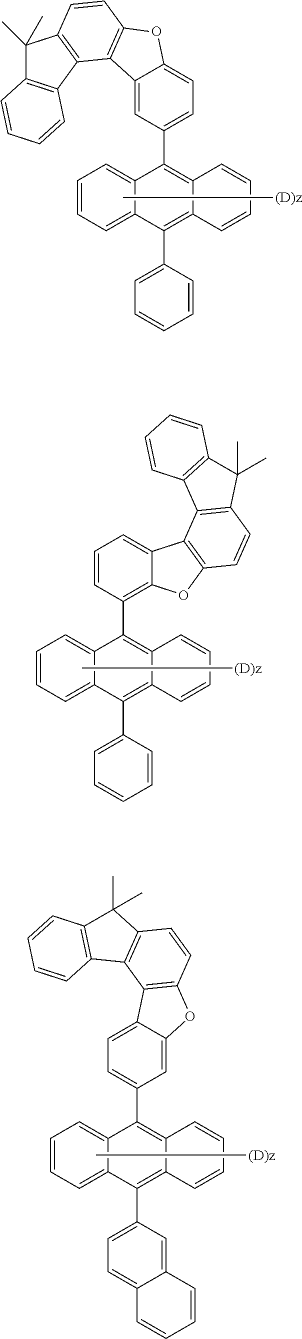

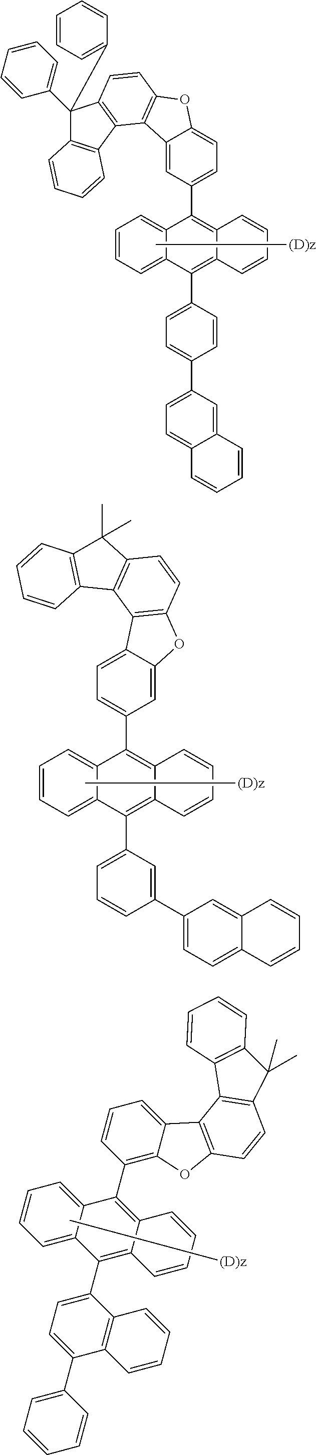

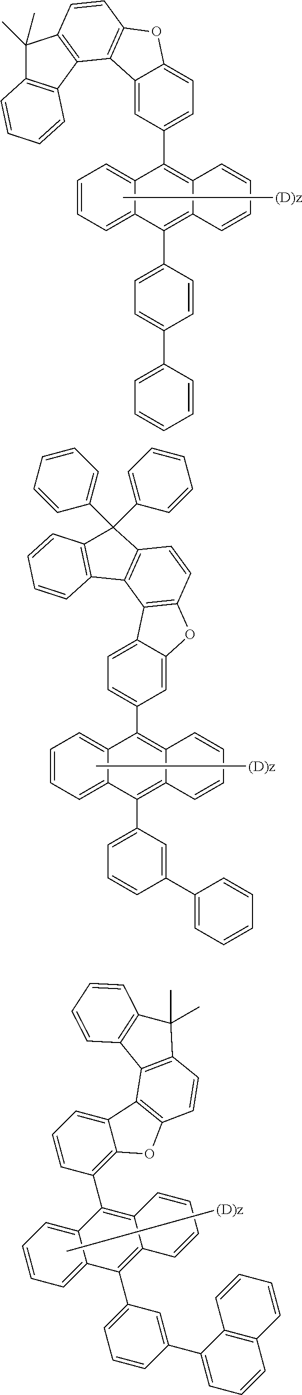

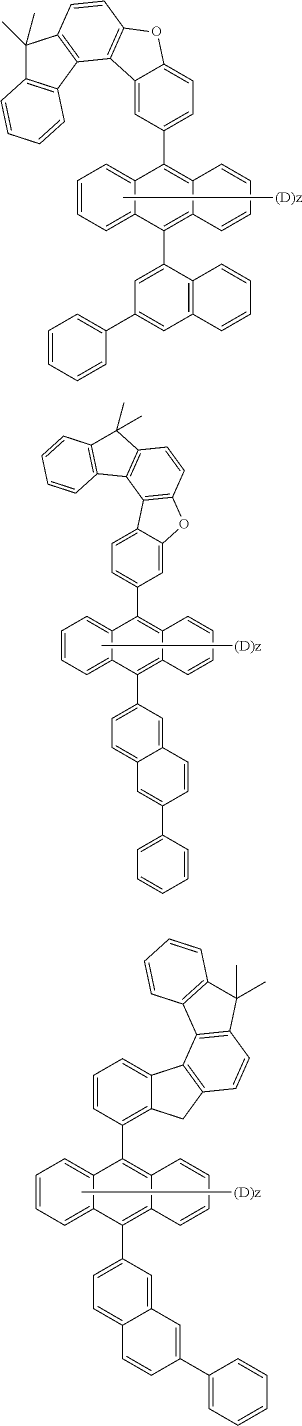

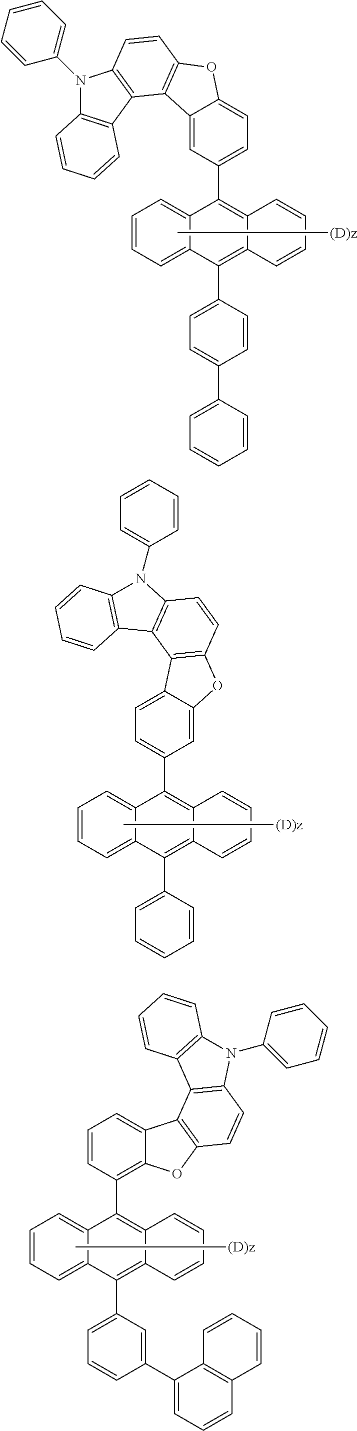

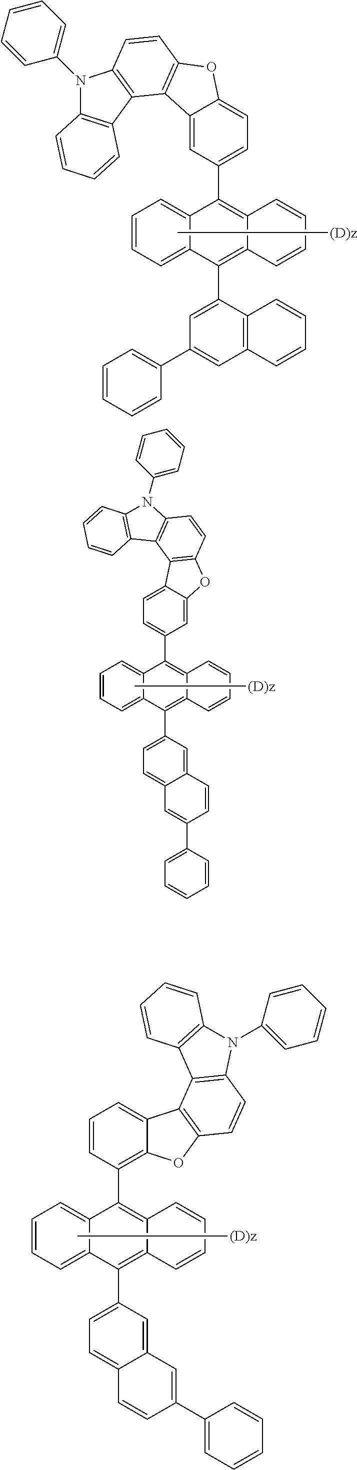

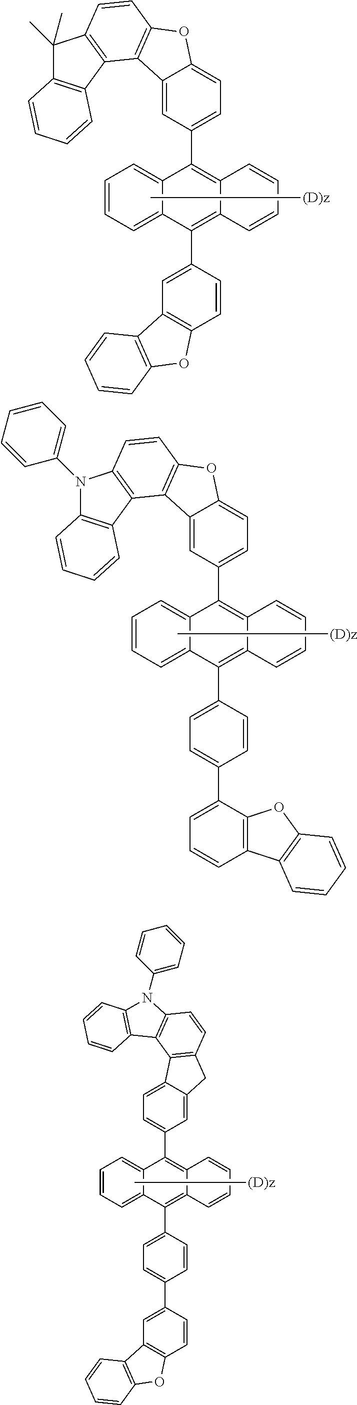

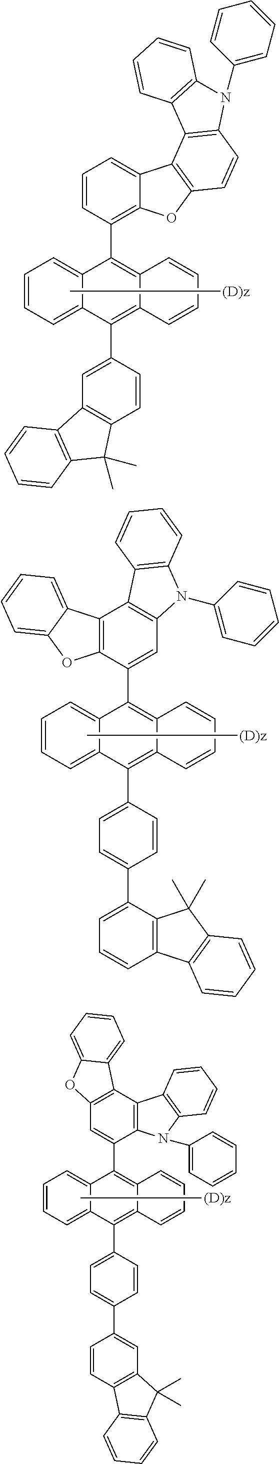

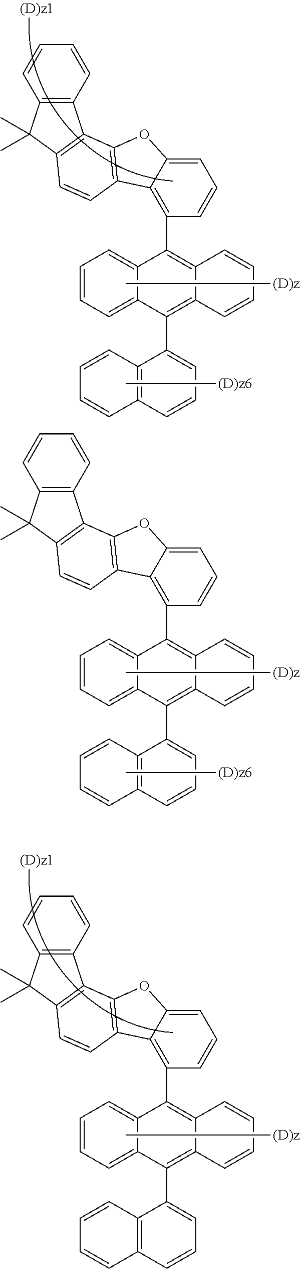

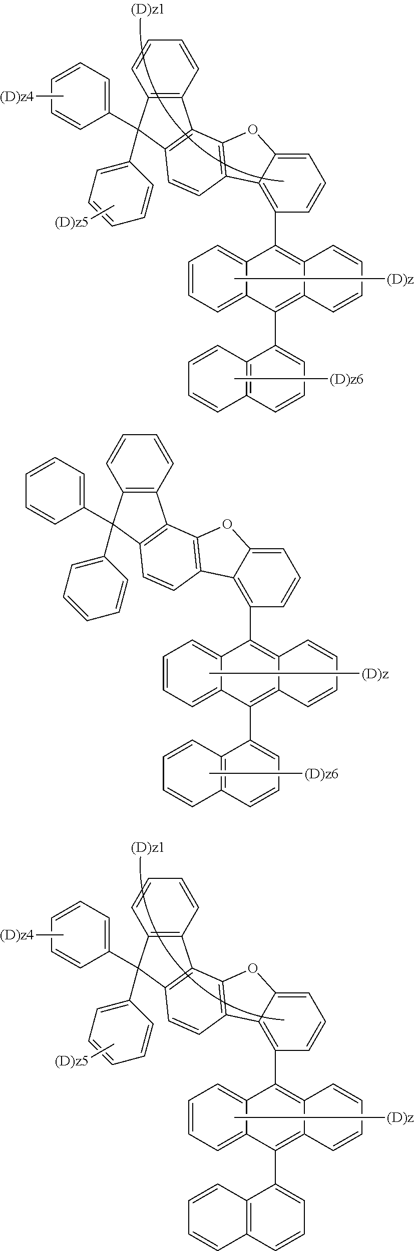

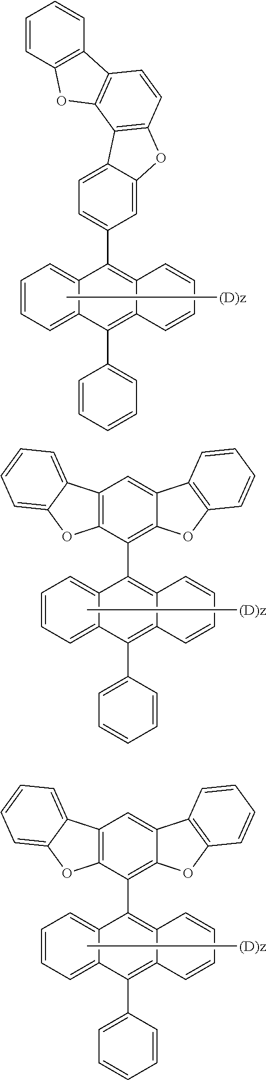

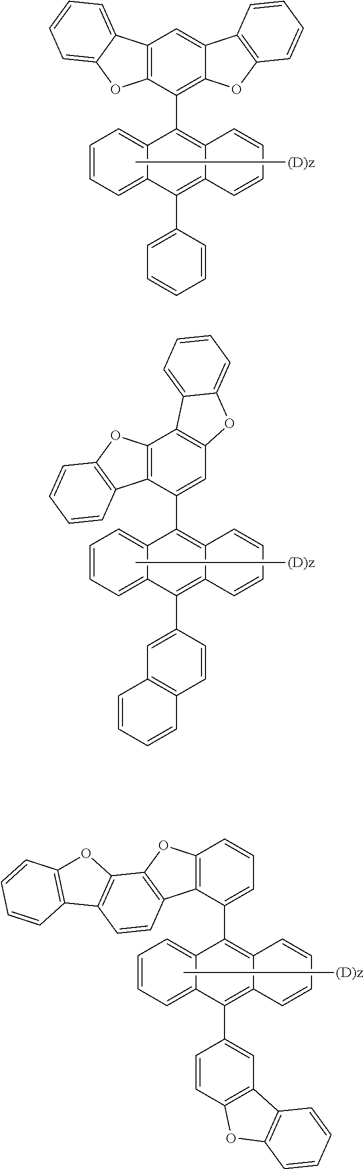

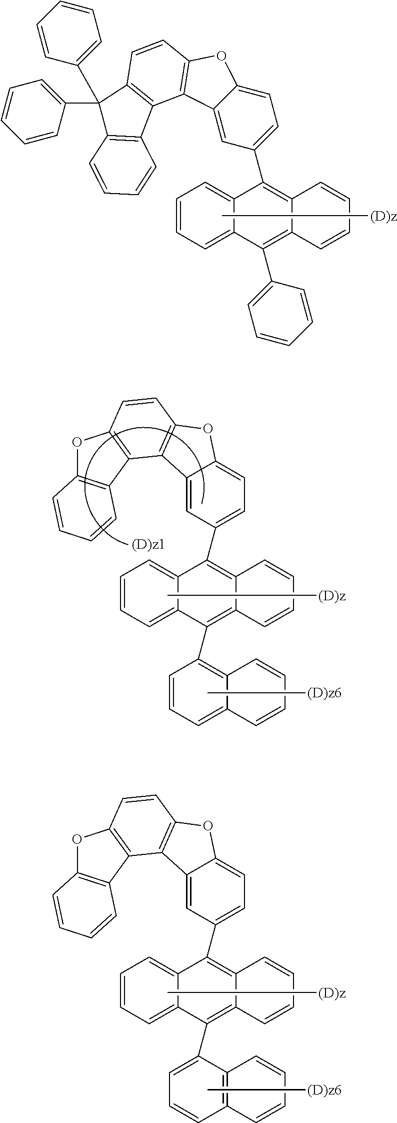

### First Luminescent Compound and Second Luminescent Compound

In the organic EL device 1B, the first emitting layer 51 also preferably includes a first luminescent compound. The first luminescent compound is preferably a fluorescent compound.

In the organic EL device 1B, the second emitting layer 52 also preferably includes a second luminescent compound. The second luminescent compound is preferably a fluorescent compound.

The first luminescent compound contained in the first emitting layer 51 and the second luminescent compound contained in the second emitting layer 52 are mutually the same or different.

Examples of the first luminescent compound and the second luminescent compound are similar to those exemplified in the luminescent compound in the organic EL device 1A.

In the organic EL device 1B, the first luminescent compound contained in the first emitting layer 51 is preferably a compound that emits light having a maximum peak wavelength of 500 nm or less, more preferably a compound that emits light having a maximum peak wavelength in a range from 430 nm to 480 nm.

In the organic EL device 1B, the first luminescent compound contained in the first emitting layer 51 is preferably a compound that emits fluorescence having a maximum peak wavelength of 500 nm or less, more preferably a compound that emits fluorescence having a maximum peak wavelength in a range from 430 nm to 480 nm.

In a case where the first emitting layer 51 of the organic EL device 1B contains the first compound and the first luminescent compound, it is preferable that the first compound is the first host material and the first luminescent compound is a first dopant material.

Moreover, in a case where the second emitting layer 52 of the organic EL device 1B contains the second compound and the second luminescent compound, it is preferable that the second compound is a second host material and the second luminescent compound is a second dopant material.

In the organic EL device 1B, it is preferable that the second emitting layer 52 contains the second host material and a triplet energy of the first host material T₁(H1) and a triplet energy of the second host material T₁(H2) satisfy a relationship of a numerical formula (Numerical Formula 1) below.

T₁(H1)>T₁(H2) (Numerical Formula 1)

Conventionally, triplet-triplet annihilation (occasionally referred to as TTA) is known as a technique for enhancing the luminous efficiency of the organic EL device. TTA is a mechanism in which triplet excitons collide with one another to generate singlet excitons. The TTA mechanism is occasionally also referred to as a TTF mechanism as described in International Publication No. WO2010/134350.

The TTF phenomenon will be described. Holes injected from an anode and electrons injected from a cathode are recombined in an emitting layer to generate excitons. As for the spin state, as is conventionally known, singlet excitons account for 25% and triplet excitons account for 75%. In a conventionally known fluorescent device, light is emitted when singlet excitons of 25% are relaxed to the ground state. The remaining triplet excitons of 75% are returned to the ground state without emitting light through a thermal deactivation process. Accordingly, the theoretical limit value of the internal quantum efficiency of the conventional fluorescent device is believed to be 25%.

The behavior of triplet excitons generated within an organic substance has been theoretically examined. According to S. M. Bachilo et al. (J. Phys. Chem. A, 104, 7711 (2000)), assuming that high-order excitons such as quintet excitons are quickly returned to triplet excitons, triplet excitons (hereinafter abbreviated as 3A′) collide with one another with an increase in density thereof, whereby a reaction shown by the following formula occurs. In the formula, 1A represents the ground state and 1A′ represents the lowest singlet excitons.

3A*+3A*→(4/9)¹A+(1/9)¹A*+(13/9)³A*

In other words, 53A*→4¹A+1A′ is satisfied, and it is expected that, among triplet excitons initially generated, which account for 75%, one fifth thereof (i.e., 20%) is changed to singlet excitons. Accordingly, the amount of singlet excitons which contribute to emission is 40%, which is a value obtained by adding 15% (75%×(1/5)=15%) to 25%, which is the amount ratio of initially generated singlet excitons. At this time, a ratio of luminous intensity derived from TTF (TTF ratio) relative to the total luminous intensity is 15/40, i.e., 37.5%. Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(1/2)=37.5%) to 25% (the amount ratio of initially generated singlet excitons). At this time, the TTF ratio is 37.5/62.5=60%.

In the organic EL device according to the exemplary embodiment, it is considered that triplet excitons generated by recombination of holes and electrons in the first emitting layer and present on an interface between the first emitting layer and the organic layer in direct contact therewith are not likely to be quenched even under the presence of excessive carriers on the interface between the first emitting layer and the organic layer. For instance, the presence of a recombination region locally on an interface between the first emitting layer and a hole transporting layer or an electron blocking layer is considered to cause quenching by excessive electrons. Meanwhile, the presence of a recombination region locally on an interface between the first emitting layer and the electron transporting layer or the hole blocking layer is considered to cause quenching by excessive holes.

When the organic EL device 1B includes at least two emitting layers (i.e., the first emitting layer 51 and the second emitting layer 52) satisfying a predetermined relationship, specifically, includes the first emitting layer 51 and the second emitting layer 52 so that the triplet energy of the first compound T₁ (H1) in the first emitting layer 51 and the triplet energy of the second compound T₁ (H2) in the second emitting layer 52 satisfy the relationship of the numerical formula (Numerical Formula 1), triplet excitons generated in the first emitting layer 51 can transfer to the second emitting layer 52 without being quenched by excessive carriers and be inhibited from back-transferring from the second emitting layer 52 to the first emitting layer 51. Consequently, the second emitting layer 52 exhibits the TTF mechanism to effectively generate singlet excitons, thereby improving the luminous efficiency.

Accordingly, the organic EL device 1B includes, as different regions, the first emitting layer 51 mainly generating triplet excitons and the second emitting layer 52 mainly exhibiting the TTF mechanism with triplet excitons having transferred from the first emitting layer 51, and a difference in triplet energy is provided by using a compound having a smaller triplet energy than that of the first compound in the first emitting layer as the second compound in the second emitting layer 52, thereby improving the luminous efficiency.

### Triplet Energy T₁

A method of measuring a triplet energy T₁ is exemplified by a method below.

A measurement target compound is dissolved in EPA (diethylether:isopentane:ethanol=5:5:2 in volume ratio) so as to fall within a range from 10-5 mol/L to 10⁻⁴ mol/L to prepare a solution, and the obtained solution is encapsulated in a quartz cell to provide a measurement sample. A phosphorescence spectrum (ordinate axis: phosphorescent luminous intensity, abscissa axis: wavelength) of the measurement sample is measured at a low temperature (77K). A tangent is drawn to the rise of the phosphorescence spectrum close to the short-wavelength region. An energy amount is calculated by a conversion equation (F1) below on a basis of a wavelength value λ_{edge} [nm] at an intersection of the tangent and the abscissa axis.

The calculated energy amount is defined as triplet energy T₁.

$$Conversion Equation ( F 1 ) : {T}_{1} [ e V ] = 1239 \text{.85} / {λ}_{edge}$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 (produced by Hitachi High-Technologies Corporation) is usable. The measurement apparatus is not limited thereto. A combination of a cooling unit, a low temperature container, an excitation light source and a light-receiving unit may be used for measurement.

When the first emitting layer 51 of the organic EL device 1B contains the first compound and the first luminescent compound, a singlet energy of the first compound S₁(H1) and a singlet energy of the first luminescent compound S₁(D3) preferably satisfy a relationship of a numerical formula (Numerical Formula 2) below.

$${S}_{1} ( H 1 ) > {S}_{1} ( D 3 ) \qquad ( Numerical Formula 2 )$$

### Singlet Energy S₁

A method of measuring a singlet energy S₁ with use of a solution (occasionally referred to as a solution method) is exemplified by a method below.

A toluene solution of a measurement target compound at a concentration ranging from 10⁻⁵ mol/L to 10⁻⁴ mol/L is prepared and put in a quartz cell. An absorption spectrum (ordinate axis: absorption intensity, abscissa axis: wavelength) of the thus-obtained sample is measured at a normal temperature (300K). A tangent is drawn to the fall of the absorption spectrum close to the long-wavelength region, and a wavelength value λ_{edge} (nm) at an intersection of the tangent and the abscissa axis is assigned to a conversion equation (F2) below to calculate singlet energy S₁.

$$Conversion Equation ( F 2 ) : {S}_{1} [ e V ] = 1239 \text{.85} / λedge$$

Any apparatus for measuring the absorption spectrum is usable. For instance, a spectrophotometer (U3310 produced by Hitachi, Ltd.) is usable.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

In a case where the second emitting layer 52 of the organic EL device 1B contains the second compound and the second luminescent compound, a singlet energy of the second compound S₁(H2) and a singlet energy of the second luminescent compound S₁(D4) preferably satisfy a relationship of a numerical formula (Numerical Formula 3) below.

$${S}_{1} ( H 2 ) > {S}_{1} ( D 4 ) \qquad ( Numerical Formula 3 )$$

The first emitting layer 51 and the second emitting layer 52 preferably contain no phosphorescent material (no dopant material).

Further, the first emitting layer 51 and the second emitting layer 52 preferably contain no heavy-metal complex and no phosphorescent rare earth metal complex. Here, examples of the heavy-metal complex include an iridium complex, osmium complex, and platinum complex.

Furthermore, the first emitting layer 51 and the second emitting layer 52 also preferably contain no metal complex.

### Film Thickness of Emitting Layer

A film thickness of each of the first emitting layer 51 and the second emitting layer 52 in the organic EL device 1B is preferably in a range from 5 nm to 50 nm, also preferably in a range from 7 nm to 50 nm, and also preferably in a range from 10 nm to 50 nm. A film thickness of the emitting layer of 5 nm or more facilitates the formation of the emitting layer and the adjustment of chromaticity. A film thickness of the emitting layer of 50 nm or less easily inhibits an increase in drive voltage.

### Content Ratios of Compounds in Emitting Layer

In a case where the first emitting layer 51 contains the first compound and the first luminescent compound, content ratios of the first compound and the first luminescent compound in the first emitting layer 51 preferably fall, for instance, within ranges below.

The content ratio of the first compound falls within, preferably, in a range from 80 mass % to 99 mass %, more preferably in a range from 90 mass % to 99 mass %, and still more preferably in a range from 95 mass % to 99 mass %.

The content ratio of the first luminescent compound falls within, preferably, in a range from 1 mass % to 10 mass %, more preferably in a range from 1 mass % to 7 mass %, and still more preferably in a range from 1 mass % to 5 mass %.

The upper limit of the total of the content ratios of the first compound and the first luminescent compound in the first emitting layer 51 is 100 mass %.

In the exemplary embodiment, the first emitting layer 51 may further contain any other material than the first compound and the first luminescent compound.

The first emitting layer 51 may contain a single type or two or more types of the first compound. The first emitting layer 51 may contain a single type or two or more types of the first luminescent compound.

In a case where the second emitting layer 52 contains the second compound and the second luminescent compound, content ratios of the second compound and the second luminescent compound in the second emitting layer 52 preferably fall, for instance, within ranges below.

The content ratio of the second compound falls within, preferably, in a range from 80 mass % to 99 mass %, more preferably in a range from 90 mass % to 99 mass %, and still more preferably in a range from 95 mass % to 99 mass %.

The content ratio of the second luminescent compound falls within, preferably, in a range from 1 mass % to 10 mass %, more preferably in a range from 1 mass % to 7 mass %, and still more preferably in a range from 1 mass % to 5 mass %.

The upper limit of the total of the content ratios of the second compound and the second luminescent compound in the second emitting layer 52 is 100 mass %.

In the exemplary embodiment, the second emitting layer 52 may further contain any other material than the second compound and the second luminescent compound.

The second emitting layer 52 may contain a single type or two or more types of the second compound. The second emitting layer 52 may contain a single type or two or more types of the second luminescent compound.

In the organic EL device 1B, the first emitting layer 51 and the second emitting layer 52 are also preferably in direct contact with each other.

Herein, a layer arrangement in which the first emitting layer 51 and the second emitting layer 52 are in direct contact with each other in the organic EL device 1B may include one of arrangements (LS1), (LS2) and (LS3) below.

(LS1) An arrangement in which a region containing both the first compound as a host material (hereinafter occasionally referred to as a first host material) and the second compound as a host material (hereinafter occasionally referred to as a second host material) is generated in a process of vapor-depositing the compound of the first emitting layer 51 and vapor-depositing the compound of the second emitting layer 52, and is present on the interface between the first emitting layer 51 and the second emitting layer 52.

(LS2) An arrangement in which in a case of containing a luminescent compound in the first emitting layer 51 and the second emitting layer 52, a region containing the first host material, the second host material and the luminescent compound is generated in a process of vapor-depositing the compound of the first emitting layer 51 and vapor-depositing the compound of the second emitting layer 52, and is present on the interface between the first emitting layer 51 and the second emitting layer 52.

(LS3) An arrangement in which in a case of containing a luminescent compound in the first emitting layer 51 and the second emitting layer 52, a region containing the luminescent compound, a region containing the first host material, or a region containing the second host material is generated in a process of vapor-depositing the compound of the first emitting layer 51 and vapor-depositing the compound of the second emitting layer 52, and is present on the interface between the first emitting layer 51 and the second emitting layer 52.

In a case where the organic EL device 1B includes a third emitting layer, the first emitting layer 51 and the second emitting layer 52 are preferably in direct contact with each other and the second emitting layer 52 and the third emitting layer are preferably in direct contact with each other.

A layer arrangement in which the second emitting layer 52 and the third emitting layer are in direct contact with each other in the organic EL device 1B may include one of arrangements (LS4), (LS5) and (LS6) below.

(LS4) An arrangement in which a region containing both the second host material and a third host material (host material contained in the third emitting layer) is generated in a process of vapor-depositing the compound of the second emitting layer 52 and vapor-depositing the compound of the third emitting layer, and is present on the interface between the second emitting layer 52 and the third emitting layer.

(LS5) An arrangement in which in a case of containing a luminescent compound in the second emitting layer 52 and the third emitting layer, a region containing the second host material, the third host material and the luminescent compound is generated in a process of vapor-depositing the compound of the second emitting layer 52 and vapor-depositing the compound of the third emitting layer, and is present on the interface between the second emitting layer 52 and the third emitting layer.

(LS6) An arrangement in which in a case of containing a luminescent compound in the second emitting layer 52 and the third emitting layer, a region containing the luminescent compound, a region containing the second host material, or a region containing the third host material is generated in a process of vapor-depositing the compound of the second emitting layer 52 and vapor-depositing the compound of the third emitting layer, and is present on the interface between the second emitting layer 52 and the third emitting layer.

Also preferably, the organic EL device 1B further includes an interposed layer.

When the organic EL device 1B includes an interposed layer, the interposed layer is preferably disposed between the first emitting layer 51 and the second emitting layer 52.

### Interposed Layer

The interposed layer is preferably a non-doped layer. The interposed layer preferably contains no metal atom.

The interposed layer contains an interposed layer material. The interposed layer material is preferably not a luminescent compound.

The interposed layer material, which is not particularly limited, is preferably any other material than the luminescent compound.

Examples of the interposed layer material include: 1) a heterocyclic compound such as an oxadiazole derivative, benzimidazole derivative, or phenanthroline derivative; 2) a fused aromatic compound such as a carbazole derivative, anthracene derivative, phenanthrene derivative, pyrene derivative or chrysene derivative; and 3) an aromatic amine compound such as a triarylamine derivative or a fused polycyclic aromatic amine derivative.

The interposed layer material may be one or both of the first compound contained in the first emitting layer 51 and the second compound contained in the second emitting layer 52.

When the interposed layer contains a plurality of interposed layer materials, the content of each interposed layer material is preferably 10 mass % or more with respect to the total mass of the interposed layer.

The interposed layer contains the interposed layer material preferably at 60 mass % or more, more preferably at 70 mass % or more, still more preferably at 80 mass % or more, still further more preferably at 90 mass % or more, and yet still further more preferably at 95 mass % or more, with respect to the total mass of the interposed layer.

The interposed layer may contain a single type of interposed layer material or two or more types of interposed layer materials.

When the interposed layer contains two or more types of interposed layer materials, the upper limit of the total of the content ratios of the two or more types of interposed layer materials is 100 mass %.

It should be noted that the interposed layer of the exemplary embodiment may further contain any other material than the interposed layer material.

The interposed layer may be provided in the form of a single layer or a laminate of two or more layers.

A film thickness of the interposed layer, which is not particularly limited, is preferably in a range from 3 nm to 15 nm, more preferably in a range from 5 nm to 10 nm per layer.

The arrangements of respective layers common to the organic EL devices 1A and 1B will be further described below. It should be noted that the reference numerals are occasionally omitted below.

### Substrate

The substrate 2 is used as a support for the organic EL device. For instance, glass, quartz, plastics and the like are usable for the substrate 2. A flexible substrate is also usable. The flexible substrate, which is a bendable substrate, is exemplified by a plastic substrate. Examples of a material for the plastic substrate include polycarbonate, polyarylate, polyethersulfone, polypropylene, polyester, polyvinyl fluoride, polyvinyl chloride, polyimide, and polyethylene naphthalate. Further, an inorganic vapor deposition film is also usable.

### Anode

Metal, an alloy, an electrically conductive compound, a mixture thereof, or the like having a large work function (specifically, 4.0 eV or more) is preferably used as the anode 3 formed on the substrate. Specific examples of the material include indium tin oxide (ITO), indium oxide-tin oxide containing silicon or silicon oxide, indium oxide-zinc oxide, indium oxide containing tungsten oxide and zinc oxide, and graphene. In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method. For instance, the indium oxide-zinc oxide can be formed into a film by the sputtering method using a target in which zinc oxide in a range from 1 mass % to 10 mass % is added to indium oxide. Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide. In addition, the anode may be formed by a vacuum deposition method, a coating method, an inkjet method, a spin coating method or the like.

Among the organic layers formed on the anode, since the hole injecting layer adjacent to the anode is formed of a composite material into which holes are easily injectable irrespective of the work function of the anode, a material usable as an electrode material (e.g., metal, an alloy, an electroconductive compound, a mixture thereof, and the elements belonging to the group 1 or 2 of the periodic table) is also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AILi) including the alkali metal or the alkaline earth metal, a rare earth metal such as europium (Eu) and ytterbium (Yb), alloys including the rare earth metal are also usable for the anode. It should be noted that the vacuum deposition method and the sputtering method are usable for forming the anode using the alkali metal, alkaline earth metal and the alloy thereof. Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode 4. Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AILi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof. Further, when a silver paste is used for the cathode, the coating method and the inkjet method are usable.

By providing the electron injecting layer, various conductive materials such as Al, Ag, ITO, graphene, and indium oxide-tin oxide containing silicon or silicon oxide may be used for forming the cathode regardless of the work function. The conductive materials can be formed into a film using the sputtering method, inkjet method, spin coating method, or the like.

### Hole Injecting Layer

The hole injecting layer 61 is a layer containing a substance exhibiting a high hole injectability. Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, the examples of the substance exhibiting a high hole injectability include: aromatic amine compounds, which are low-molecule organic compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}—N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazole-3-yl)-N-phenylamino]-9-phenylcarbazole (abbreviation: PCzPCA1), 3,6-bis[N-(9-phenylcarbazole-3-yl)-N-phenylamino]-9-phenylcarbazole (abbreviation: PCzPCA2), and thereof 3-[N-(1-naphthyl)-N-(9-phenylcarbazole-3-yl)amino]-9-phenylcarbazole (abbreviation: PCzPCN1); and dipyrazino [2,3-f: 20,30-h]quinoxaline-2,3,6,7,10,11-hexacarbonitrile (HAT-CN), which is also a low-molecule organic compound.

In addition, a high polymer compound (e.g., oligomer, dendrimer and polymer) is usable as the substance exhibiting a high hole injectability. Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamide] (abbreviation: PTPDMA), and poly[N,N′-bis(4-butylphenyl)-N,N′-bis(phenyl)benzidine] (abbreviation: Poly-TPD). Moreover, an acid-added high polymer compound such as poly(3,4-ethylenedioxythiophene)/poly(styrene sulfonic acid) (PEDOT/PSS) and polyaniline/poly(styrene sulfonic acid) (PAni/PSS) are also usable.

### Hole Transporting Layer

The hole transporting layer 62 is a layer containing a substance exhibiting a high hole transportability. An aromatic amine compound, carbazole derivative, anthracene derivative and the like are usable for the hole transporting layer 62. Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and thereof 4,4′-bis[N-(spiro-9,9′-bifluorene-2-yl)-N-phenylamino]biphenyl (abbreviation: BSPB). The above-described substances mostly have a hole mobility of 10⁻⁶ cm²/(V·s) or more.

For the hole transporting layer 62, a carbazole derivative such as CBP, thereof 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used. A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used. It should be noted that the layer containing the substance exhibiting a high hole transportability may be not only a single layer but also a laminate of two or more layers formed of the above substance(s).

### Specific Examples of Hole Transporting Zone Material

Specific examples of the hole transporting zone material include the following compounds. However, the invention is by no means limited to the specific examples of the hole transporting zone material.

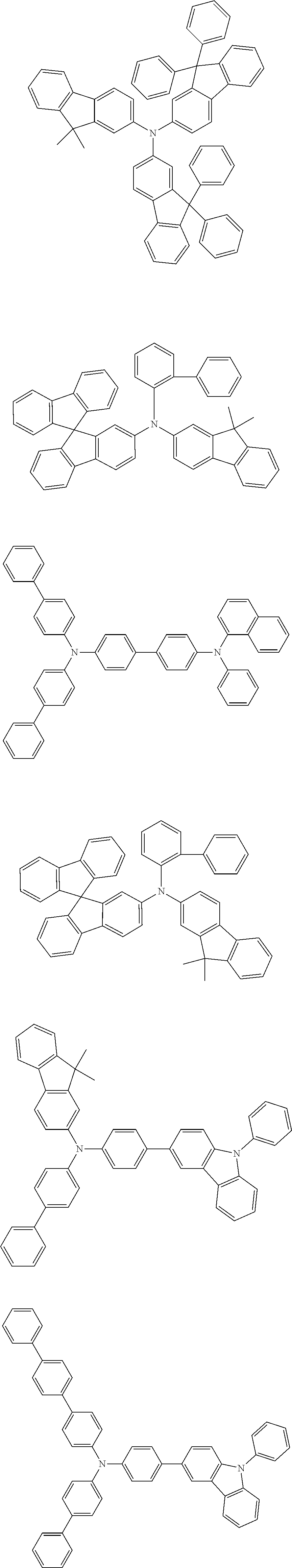

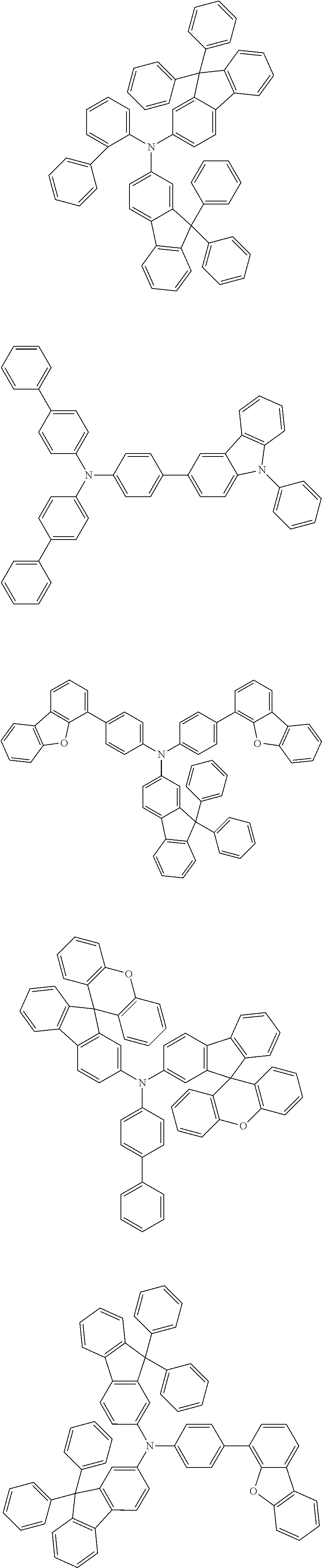

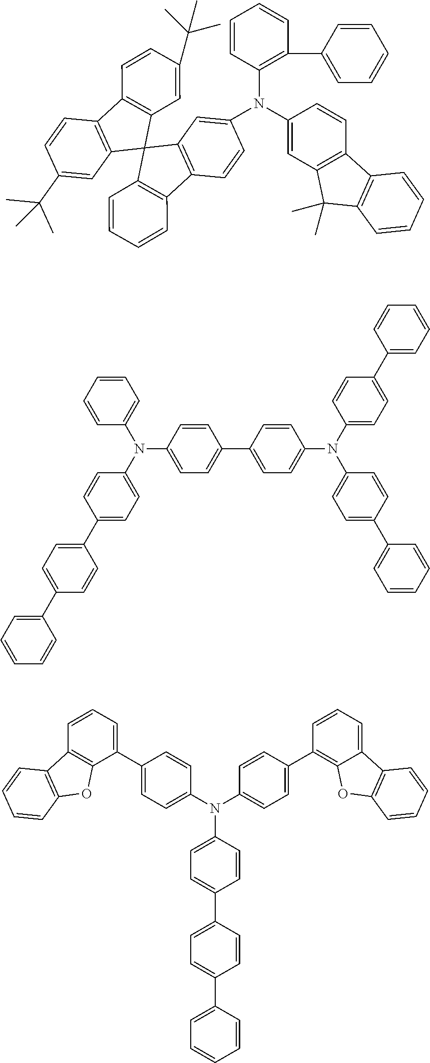 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

### Electron Transporting Layer

The electron transporting layer 71 is a layer containing a substance exhibiting a high electron transportability. For the electron transporting layer 71, 1) a metal complex such as an aluminum complex, beryllium complex, and thereof zinc complex, 2) a hetero aromatic compound such as imidazole derivative, benzimidazole derivative, azine derivative, carbazole derivative, and phenanthroline derivative, and 3) a high polymer compound are usable. Specifically, as a low-molecule organic compound, a metal complex such as Alq, tris(4-methyl-8-quinolinolato)aluminum (abbreviation: Almq₃), bis(10-hydroxybenzo[h]quinolinato) beryllium (abbreviation: BeBq₂), BAlq, Znq, ZnPBO and ZnBTZ is usable. In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable. In the exemplary embodiment, a benzimidazole compound is suitably usable. The above-described substances mostly have an electron mobility of 10⁻⁶ cm²/Vs or more. It should be noted that any other substance than the above substances may be used for the electron transporting layer as long as the substance exhibits a higher electron transportability than the hole transportability. The electron transporting layer may be a single layer or a laminate of two or more layers formed of the above substance.

Further, a high polymer compound is usable for the electron transporting layer 71. For instance, poly[(9,9-dihexylfluorene-2,7-diyl)-co-(pyridine-3,5-diyl)] (abbreviation: PF-Py), and poly[(9,9-dioctylfluorene-2,7-diyl)-co-(2,2′-bipyridine-6,6′-diyl)] (abbreviation: PF-BPy) are usable.

### Specific Examples of Electron Transporting Material

Specific examples of the electron transporting material usable for the electron transporting layer include the following compounds. However, the invention is by no means limited to the specific examples of the electron transporting material.

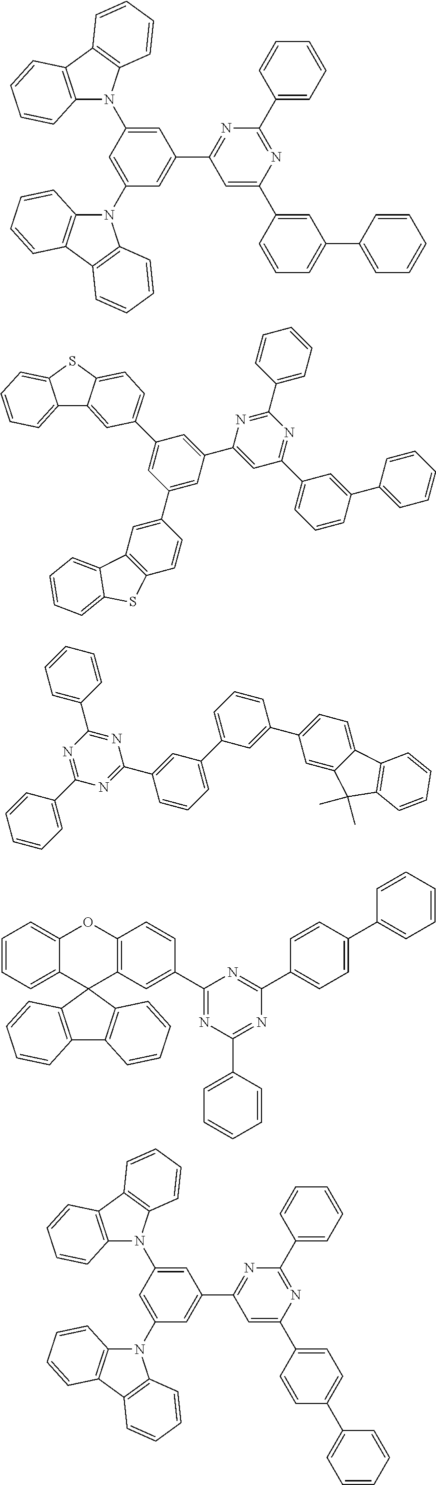

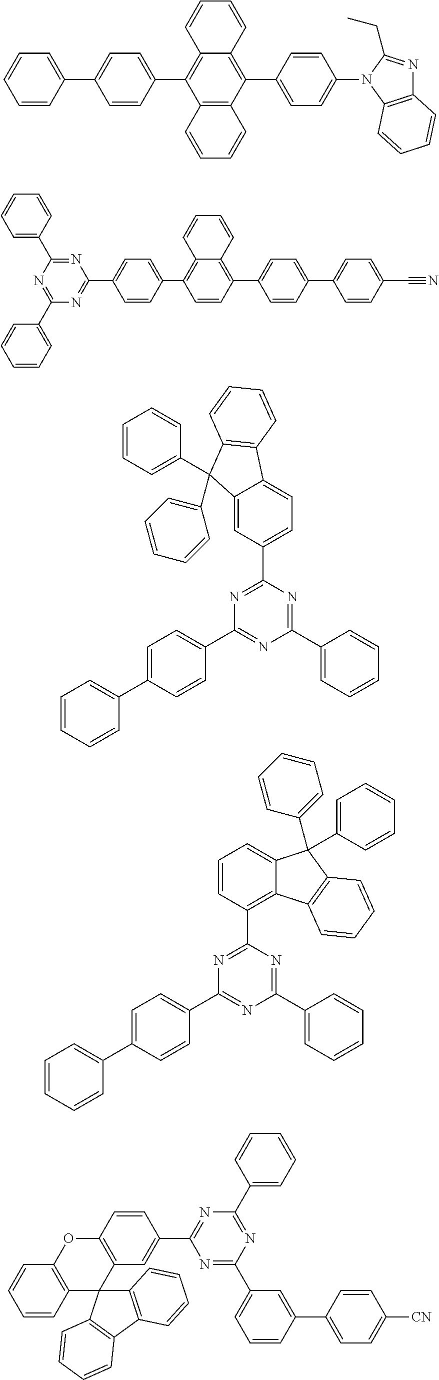 [SMILES: CCc1nc2ccccc2n1-c1ccc(-c2c3ccccc3c(-c3ccc(-c4ccccc4)cc3)c3ccccc23)cc1 ; N#Cc1ccc(-c2ccc(-c3ccc(-c4ccc(-c5nc(-c6ccccc6)nc(-c6ccccc6)n5)cc4)c4ccccc34)cc2)cc1 ; c1ccc(-c2ccc(-c3nc(-c4ccccc4)nc(-c4ccc5c(c4)C(c4ccccc4)(c4ccccc4)c4ccccc4-5)n3)cc2)cc1 ; c1ccc(-c2ccc(-c3nc(-c4ccccc4)nc(-c4cccc5c4-c4ccccc4C5(c4ccccc4)c4ccccc4)n3)cc2)cc1 ; N#Cc1ccc(-c2cccc(-c3nc(-c4ccccc4)nc(-c4ccc5c(c4)C4(c6ccccc6O5)c5ccccc5-c5ccccc54)n3)c2)cc1]

### Electron Injecting Layer

The electron injecting layer 72 is a layer containing a substance exhibiting a high electron injectability. Examples of a material for the electron injecting layer 72 include an alkali metal, alkaline earth metal and a compound thereof, examples of which include lithium (Li), cesium (Cs), calcium (Ca), lithium fluoride (LiF), cesium fluoride (CsF), calcium fluoride (CaF₂), and thereof lithium oxide (LiOx). In addition, the alkali metal, alkaline earth metal or the compound thereof may be added to the substance exhibiting the electron transportability in use. Specifically, for instance, magnesium (Mg) added to Alq may be used. In this case, the electrons can be more efficiently injected from the cathode.

Alternatively, the electron injecting layer 72 may be provided by a composite material in a form of a mixture of the organic compound and the electron donor. Such a composite material exhibits excellent electron injectability and electron transportability since electrons are generated in the organic compound by the electron donor. In this case, the organic compound is preferably a material excellent in transporting the generated electrons. Specifically, the above examples (e.g., the metal complex and the hetero aromatic compound) of the substance forming the electron transporting layer are usable. As the electron donor, any substance exhibiting electron donating property to the organic compound is usable. Specifically, the electron donor is preferably alkali metal, alkaline earth metal and rare earth metal such as lithium, cesium, magnesium, calcium, erbium and ytterbium. The electron donor is also preferably alkali metal oxide and alkaline earth metal oxide such as lithium oxide, calcium oxide, and barium oxide. Moreover, a Lewis base such as magnesium oxide is usable. Further, the organic compound such as tetrathiafulvalene (abbreviation: TTF) is usable.

### Layer Formation Method

A method of forming each layer of the organic EL device in the exemplary embodiment is subject to no limitation except for the above particular description. However, known methods of dry film-forming such as vacuum deposition, sputtering, plasma or ion plating and wet film-forming such as thereof spin coating, dipping, flow coating or ink-jet are applicable.

### Film Thickness

The film thickness of each layer of the organic layer of the organic EL device in the exemplary embodiment is not limited unless otherwise specified in the above. In general, the thickness preferably ranges from several nanometers to 1 μm because an excessively small film thickness is likely to cause defects (e.g. pin holes) and an excessively large thickness leads to the necessity of applying high voltage and consequent reduction in efficiency.

According to the exemplary embodiment, an organic electroluminescence device with an improved luminous efficiency can be provided.

### Fourth Exemplary Embodiment

### Electronic Device

An electronic device according to a fourth exemplary embodiment includes the organic electroluminescence device according to the third exemplary embodiment. Examples of the electronic device include a display device and a light-emitting unit. Examples of the display device include a display component (e.g., an organic EL panel module), TV, mobile phone, tablet and personal computer. Examples of the light-emitting unit include an illuminator and a vehicle light. The light-emitting apparatus also can be used for a display device, for instance, as a backlight of the display device.

### Modification of Embodiments

The scope of the invention is not limited by the above exemplary embodiments but includes any modification and improvement as long as such modification and improvement are compatible with the invention.

For instance, the number of the emitting layers included in the organic EL device is not limited to one or two, but three or more emitting layers may be layered. In a case where the organic EL device includes a plurality of emitting layers (two or more emitting layers), it is only required that at least two emitting layers (first and second emitting layers) satisfy the conditions described in the above exemplary embodiments. For instance, the rest of the emitting layers may be a fluorescent emitting layer or a phosphorescent emitting layer with use of emission caused by electron transfer from the triplet excited state directly to the ground state.

In a case where the organic EL device includes a plurality of emitting layers, these emitting layers may be mutually adjacently provided, or may form a so-called tandem organic EL device, in which a plurality of emitting units are layered via an intermediate layer.

For instance, a blocking layer may be provided adjacent to at least one of a side of the emitting layer close to the anode or a side of the emitting layer close to the cathode. The blocking layer is preferably provided in contact with the emitting layer to block at least one of holes, electrons, or excitons.

For instance, when the blocking layer is provided in contact with the side of the emitting layer close to the cathode, the blocking layer permits transport of electrons, and blocks holes from reaching a layer provided closer to the cathode (e.g., the electron transporting layer) beyond the blocking layer. When the organic EL device includes the electron transporting layer, the blocking layer is preferably interposed between the emitting layer and the electron transporting layer.

When the blocking layer is provided in contact with the side of the emitting layer close to the anode, the blocking layer permits transport of holes and blocks electrons from reaching a layer provided closer to the anode (e.g., the hole transporting layer) beyond the blocking layer. When the organic EL device includes the hole transporting layer, the blocking layer is preferably interposed between the emitting layer and the hole transporting layer.

Alternatively, the blocking layer may be provided adjacent to the emitting layer so that the excitation energy does not leak out from the emitting layer toward neighboring layer(s). The blocking layer blocks excitons generated in the emitting layer from being transferred to a layer(s) (e.g., the electron transporting layer and the hole transporting layer) closer to the electrode(s) beyond the blocking layer.

The emitting layer is preferably bonded with the blocking layer.

The specific structure, shape, and the like of the components in the invention may be designed in any manner as long as an object of the invention can be achieved.

### EXAMPLES

The invention will be described in further detail with reference to Examples. The scope of the invention is by no means limited to Examples.

### Compounds

Structures of a compound represented by the formula (1) and used for producing organic EL devices in Examples 1 to 4 are given below.

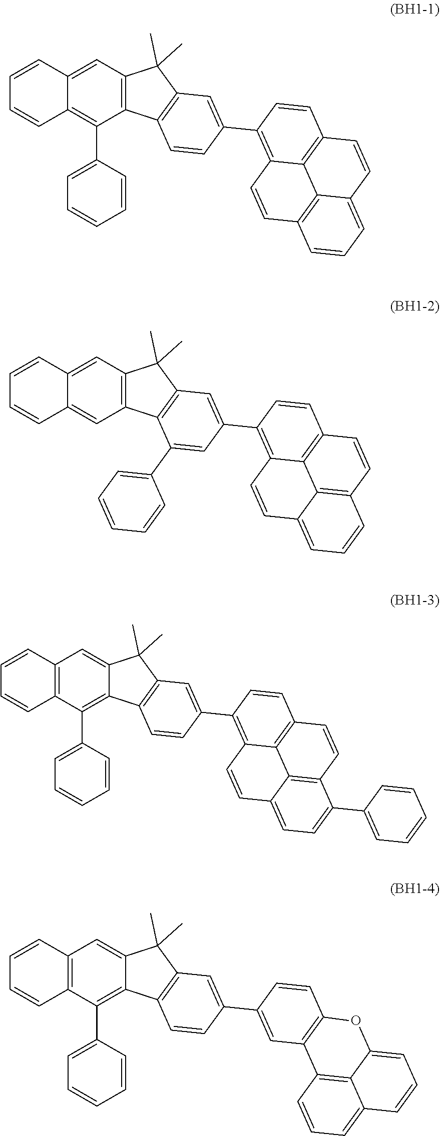

A structure of a comparative compound used for producing an organic EL device in Comparative 1 is given below.

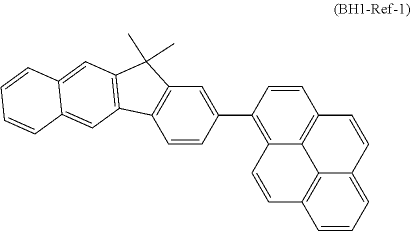 [SMILES: CC1(C)c2cc(-c3ccc4ccc5cccc6ccc3c4c56)ccc2-c2cc3ccccc3cc21]

Structures of other compounds used for producing the organic EL devices in Examples 1 to 4 and Comparative 1 are given below.

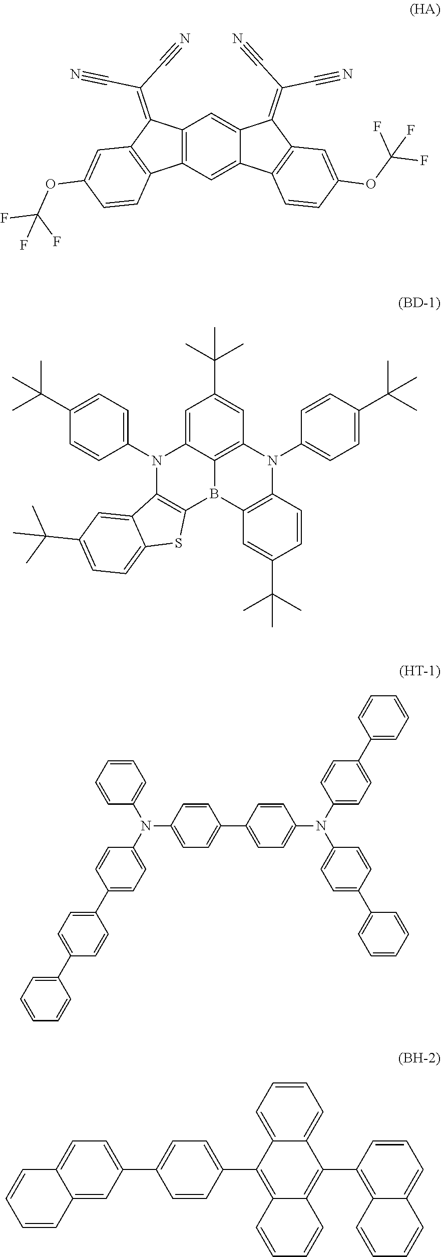

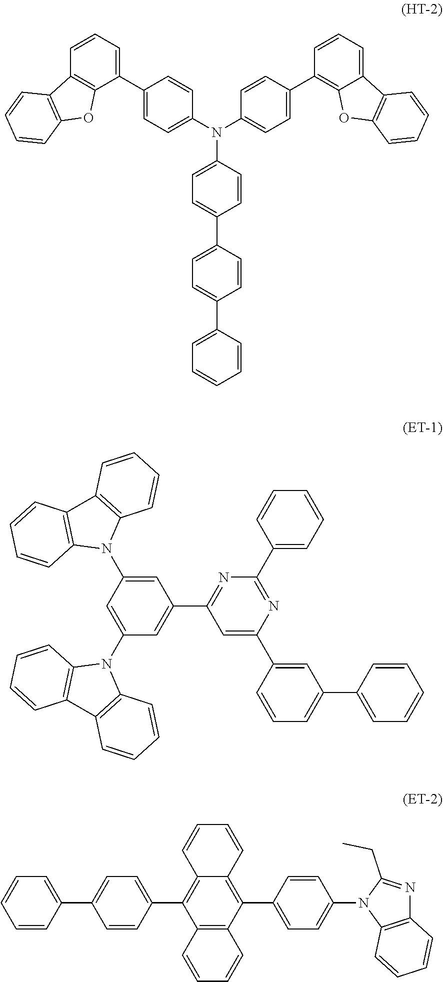 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cccc(-c3cc(-c4cc(-n5c6ccccc6c6ccccc65)cc(-n5c6ccccc6c6ccccc65)c4)nc(-c4ccccc4)n3)c2)cc1 ; CCc1nc2ccccc2n1-c1ccc(-c2c3ccccc3c(-c3ccc(-c4ccccc4)cc3)c3ccccc23)cc1]

### Example 1

A glass substrate (size: 25 mm×75 mm×1.1 mm thick, produced by Geomatec Co., Ltd.) having an indium tin oxide (ITO) transparent electrode (anode) was ultrasonic-cleaned in isopropyl alcohol for five minutes, and then UV-ozone-cleaned for 30 minutes. The film thickness of the ITO transparent electrode was 130 nm.

After the glass substrate having the transparent electrode line was cleaned, the glass substrate was mounted on a substrate holder of a vacuum deposition apparatus. Firstly, a compound HA was vapor-deposited on a surface of the glass substrate where the transparent electrode line was provided in a manner to cover the transparent electrode, thereby forming a 5-nm-thick hole injecting layer.

After forming the hole injecting layer, a compound HT-1 was vapor-deposited on the hole injecting layer to form an 80-nm-thick first hole transporting layer.

After forming the first hole transporting layer, a compound HT-2 was vapor-deposited on the first hole transporting layer to form a 10-nm-thick second hole transporting layer (also referred to as an electron blocking layer).

A compound BH1-1 as the first compound and a compound BD-1 as the first luminescent compound were co-deposited on the second hole transporting layer such that the ratio of the compound BH1-1 accounted for 98 mass % and the ratio of the compound BD-1 accounted for 2 mass %, thereby forming a 5-nm-thick first emitting layer.

A compound BH-2 as the second compound and the compound BD-1 as the second luminescent compound were co-deposited on the first emitting layer such that the ratio of the compound BH-2 accounted for 98 mass % and the ratio of the compound BD-1 accounted for 2 mass %, thereby forming a 20-nm-thick second emitting layer.

A compound ET-1 was vapor-deposited on the second emitting layer to form a 10-nm-thick first electron transporting layer (also referred to as a hole blocking layer).

A compound ET-2 was vapor-deposited on the first electron transporting layer to form a 15-nm-thick second electron transporting layer.

LiF was vapor-deposited on the second electron transporting layer to form a 1-nm-thick electron injecting layer.

Metal Al was vapor-deposited on the electron injecting layer to form an 80-nm-thick cathode.

A device arrangement of the organic EL device in Example 1 is roughly shown as follows.
ITO(130)/HA(5)/HT-1(80)/HT-2(10)/BH1-1:BD-1(5,98%:2%)/BH-2:BD-1(20,98%:2%)/ET-1(10)/ET-2(15)/LiF(1)/Al(80)

In the above-described device arrangement, numerals in parentheses represent a film thickness (unit: nm).

The numerals (98%:2%) represented by percentage in the same parentheses indicate a ratio (mass %) between the compound BH1-1 or the compound BH-2 and the compound BD-1 in the first emitting layer or the second emitting layer.

### Examples 2 to 4

Organic EL devices in Examples 2 to 4 were produced in the same manner as the organic EL device in Example 1 except that the compound BH1-1 as the first compound used for forming the first emitting layer was replaced with compounds listed in Table 1.

### Comparative 1

An organic EL device in Comparative 1 was produced in the same manner as in Example 1 except that the first compound of the first emitting layer was replaced with a compound listed in Table 1.

### Evaluation of Organic EL Devices

The organic EL devices produced in Examples 1 to 4 and Comparative 1 were evaluated as follows. Table 1 shows the evaluation results.

### External Quantum Efficiency EQE

Voltage was applied to each organic EL device such that a current density was 10 mA/cm², where spectral radiance spectrum was measured with a spectroradiometer CS-2000 (produced by Konica Minolta, Inc.). The external quantum efficiency EQE (unit: %) was calculated based on the obtained spectral radiance spectra, assuming that the spectra was provided under a Lambertian radiation. Table 1 shows “EQE (relative value)” (unit: %).

Relative values of EQE shown in Table 1 were calculated based on the measurement values of EQE in Examples and according to a numerical formula (Numerical Formula X1) below.

EQE (relative value: %)=(EQE of each Example/EQE of Comparative 1)×100 (Numerical Formula X₁)

### Maximum Peak Wavelength λp

Voltage was applied to each organic EL device such that a current density was 10 mA/cm², where spectral radiance spectrum was measured with a spectroradiometer CS-2000 (produced by Konica Minolta, Inc.). The maximum peak wavelength λp (unit: nm) was calculated from the obtained spectral radiance spectrum.

TABLE 1

| | First emitting layer | | | | | | | Second emitting layer | | | | | Device evaluation | |
|---|---|---|---|---|---|---|---|---|---|---|---|---|---|---|
| | | | | First luminscent | | | Film | Second | | | Second | Film | EQE | |
| | First compound | | | compound | | | thick- | compound | | | luminscent | thick- | (relative | |
| | | S₁ | T₁ | | S₁ | T₁ | ness | | S₁ | T₁ | compound | ness | value) | λₚ |
| | Name | [eV] | [eV] | Name | [eV] | [eV] | [nm] | Name | [eV] | [eV] | Name | [nm] | [%] | [nm] |
| Example 1 | BH1-1 | 3.13 | 2.09 | BD-1 | 2.80 | 2.45 | 5 | BH-2 | 3.01 | 1.86 | BD-1 | 20 | 105.7 | 463 |
| Example 2 | BH1-2 | 3.28 | 2.08 | BD-1 | 2.80 | 2.45 | 5 | BH-2 | 3.01 | 1.86 | BD-1 | 20 | 102 | 463 |
| Example 3 | BH1-3 | 3.05 | 2.05 | BD-1 | 2.80 | 2.45 | 5 | BH-2 | 3.01 | 1.86 | BD-1 | 20 | 103 | 463 |
| Example 4 | BH1-4 | 3.06 | 2.22 | BD-1 | 2.80 | 2.45 | 5 | BH-2 | 3.01 | 1.86 | BD-1 | 20 | 103 | 463 |
| Comparative | BH1-Ref1 | 3.15 | 2.10 | BD-1 | 2.80 | 2.45 | 5 | BH-2 | 3.01 | 1.86 | BD-1 | 20 | 100 | 464 |
| 1 | | | | | | | | | | | | | | |

The organic EL devices produced using a compound represented by the formula (1) in Examples 1 to 4 exhibited a higher luminous efficiency than the organic EL device in Comparative 1.

### Evaluation on Compounds

### Triplet Energy T₁

A measurement target compound was dissolved in EPA (diethylether:isopentane:ethanol=5:5:2 in volume ratio) at a concentration of 10 μmol/L to prepare a solution, and the solution was put in a quartz cell to provide a measurement sample. A phosphorescence spectrum (ordinate axis: phosphorescent luminous intensity, abscissa axis: wavelength) of the measurement sample was measured at a low temperature (77K). A tangent was drawn to the rise of the phosphorescence spectrum close to the short-wavelength region. An energy amount was calculated by a conversion equation (F1) below on a basis of a wavelength value λ_{edge} [nm] at an intersection of the tangent and the abscissa axis. The calculated energy amount was defined as triplet energy T₁. Table 1 shows the results.

It should be noted that the triplet energy T₁ may have an error of about plus or minus 0.02 eV depending on measurement conditions.

$$Conversion Equation ( F 1 ) : {T}_{1} [ e V ] = 1239 \text{.85} / {λ}_{edge}$$

The tangent to the rise of the phosphorescence spectrum close to the short-wavelength region is drawn as follows. While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum. An inclination of the tangent is increased along the rise of the curve (i.e., a value of the ordinate axis is increased). A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 manufactured by Hitachi High-Technologies Corporation was used.

### Singlet Energy S₁

A toluene solution of a measurement target compound at a concentration of 10 μmol/L was prepared and put in a quartz cell. An absorption spectrum (ordinate axis: absorption intensity, abscissa axis: wavelength) of the thus-obtained sample was measured at a normal temperature (300K). A tangent was drawn to the fall of the absorption spectrum close to the long-wavelength region, and a wavelength value λ_{edge} (nm) at an intersection of the tangent and the abscissa axis was assigned to a conversion equation (F2) below to calculate singlet energy S₁. Table 1 shows the results.

$$Conversion Equation ( F 2 ) : {S}_{1} [ e V ] = 1239 \text{.85} / {λ}_{edge}$$

A spectrophotometer (U3310 manufactured by Hitachi, Ltd.) was used for measuring absorption spectrum.

The tangent to the fall of the absorption spectrum close to the long-wavelength region is drawn as follows. While moving on a curve of the absorption spectrum from the local maximum value closest to the long-wavelength region, among the local maximum values of the absorption spectrum, in a long-wavelength direction, a tangent at each point on the curve is checked. An inclination of the tangent is decreased and increased in a repeated manner as the curve falls (i.e., a value of the ordinate axis is decreased). A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

### Maximum Peak Wavelength of Compounds

A maximum peak wavelength λ of each compound was measured as follows.

A toluene solution of each measurement target compound at a concentration of 5 μmol/L was prepared and put in a quartz cell. An emission spectrum (ordinate axis: luminous intensity, abscissa axis: wavelength) of the thus-obtained sample was measured at a normal temperature (300K). In Examples, the emission spectrum was measured with a spectrophotometer (device name: F-7000) manufactured by Hitachi High-Tech Corporation. It should be noted that the apparatus for measuring the emission spectrum is not limited to the apparatus used herein. A peak wavelength of the emission spectrum exhibiting the maximum luminous intensity was defined as the maximum peak wavelength A.

The maximum peak wavelength A of the compound BD-1 was 457 nm.

### Synthesis of Compound

### Synthesis Example 1

### Synthesis of Compound BH1-1

The compound BH1-1 was synthesized through a synthesis pathway below.

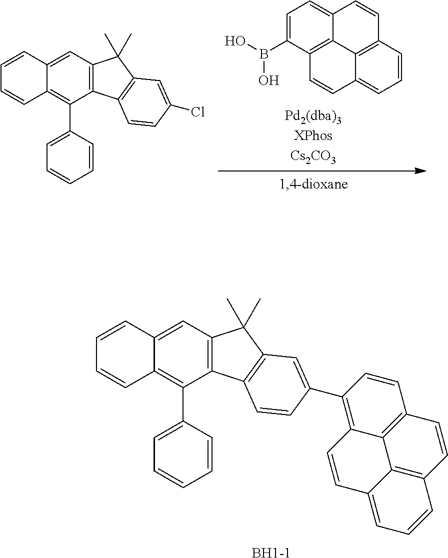

### Synthesis of Compound BH1-1

Under a nitrogen atmosphere, into a 200-mL three-necked flask, 2-chloro-11,11-dimethyl-5-phenyl-11H-benzo[b]fluorene (2.5 g, 7.04 mmol), pyrene-1-boronic acid (1.65 g, 6.71 mmol), Pd₂(dba)₃ (123 mg, 0.13 mmol), XPhos (256 mg, 0.54 mmol), cesium carbonate (4.37 g, 13.4 mmol), 1,4-dioxane (57 ml), and water (10 ml) were put. The mixture was heated to reflux for four hours. Methanol was added to the reaction solution. The deposited solid was collected by filtration. The obtained solid was purified by recrystallization and silica gel chromatography to obtain 2.5 g of a white solid. The white solid was identified as the compound BH1-1 (a yield of 71%) by ASAP-MS analysis (mass spectrometry using an atmospheric pressure solid sample probe). ASAP-MS is an abbreviation of Atmospheric Pressure solid Analysis Probe Mass Spectrometry.

### Synthesis Example 2

### Synthesis of Compound BH1-2

A compound BH1-2 was synthesized through a synthesis pathway below.

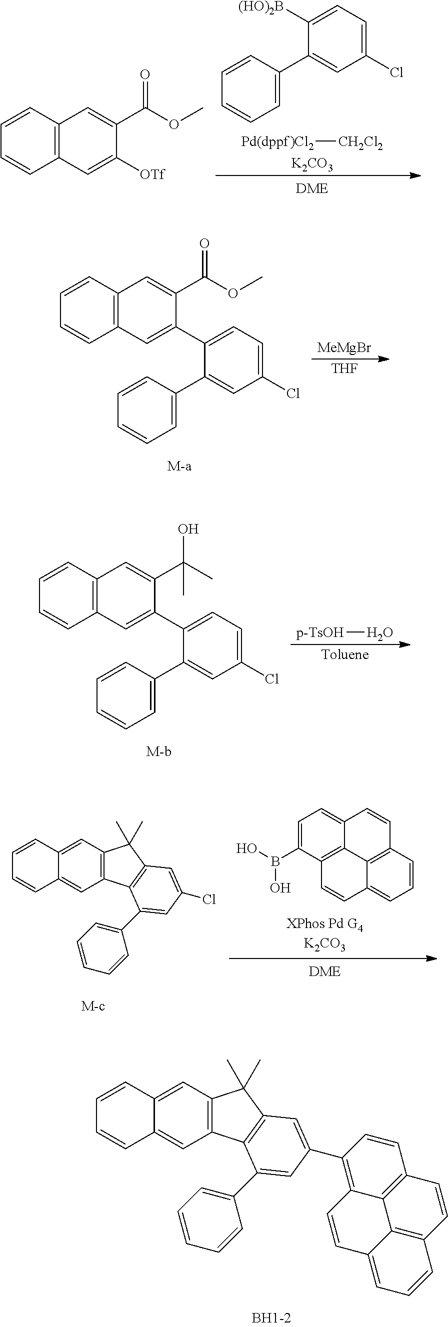

### Synthesis of Intermediate M-a

Under a nitrogen atmosphere, methyl 3-(((trifluoromethyl)sulfonyl)oxy)-2-naphthoate (9.53 g, 28.5 mmol), (5-chloro-[1,1′-biphenyl]-2-yl)boronic acid (6.68 g, 28.7 mmol), Pd(dppf)Cl₂—CH₂Cl₂ (dichloro[1,1′-bis(diphenylphosphino)ferrocene]palladium (II) dichloromethane adduct) (704 mg, 862 mmol), potassium carbonate (5.96 g, 43.1 mmol), DME (1,2-dimethoxyethane) (120 ml), and water (29 ml) were put into a 500-mL three-necked flask, and the mixture was stirred at 80 degrees C. for six hours. The reaction mixture was extracted with toluene and concentrated. The obtained brown oil was purified by silica gel chromatography to obtain 10 g of colorless oil. This colorless oil was identified as an intermediate M-a by analysis according to ASAP-MS (a yield of 93%).

### Synthesis of Intermediate M-b

Under a nitrogen atmosphere, the intermediate M-a (9.0 g, 24.1 mmol) and THF (tetrahydrofuran) (120 ml) were put into a 500-mL three-necked flask and stirred while cooling with ice. MeMgBr (methylmagnesium bromide) (3M diethyl ether solution) (24.1 ml) was added dropwise thereto, and the mixture was stirred at room temperature for five hours. The reaction mixture was quenched by adding an aqueous ammonium chloride solution, extracted with ethyl acetate, and concentrated. 7.3 g of the obtained orange solid was used as it was in the next reaction without being purified. This orange solid was identified as an intermediate M-b by analysis according to ASAP-MS.

### Synthesis of Intermediate M-c

Under a nitrogen atmosphere, the intermediate M-b (7.3 g, 19.6 mmol), p-TsOH—H₂O (p-toluenesulfonic acid monohydrate) (261 mg, 1.37 mmol), and toluene (196 ml) were put into a 500-mL three-necked flask and heated under reflux for five hours. The reaction mixture was added with water and extracted with toluene. The organic layer was concentrated and dried. The obtained orange solid was purified by silica gel chromatography and recrystallization to obtain 1.4 g of a white solid. This white solid was identified as an intermediate M-c by analysis according to ASAP-MS (a yield of 16%).

### Synthesis of Compound BH1-2

Under a nitrogen atmosphere, the intermediate M-c (1.4 g, 3.82 mmol), pyrene-1-boronic acid (940 mg, 3.82 mmol), XPhos Pd G₄ (90 mg, 0.112 mmol), potassium carbonate (1.6 g, 11.5 mmol), DME (1,2-dimethoxyethane (31 ml) and water (8 ml) were put into a 100-mL three necked flask. The mixture was heated to reflux for three hours. The reaction mixture was extracted with toluene and concentrated. The obtained yellow-brown solid was purified by silica gel chromatography and recrystallization to obtain 1.3 g of a white solid. This white solid was identified as a compound BH1-2 by analysis according to ASAP-MS (a yield of 62%).

### Synthesis Example 3

### Synthesis of Compound BH1-3

A compound BH1-3 was synthesized through a synthesis pathway below.

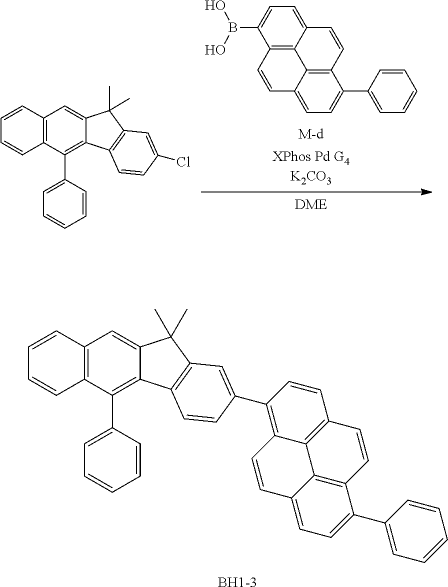

### Synthesis of Compound BH1-3

The compound BH1-3 was synthesized in the same manner as the compound BH1-2 except that the intermediate M-c was replaced by 2-chloro-11,11-dimethyl-5-phenyl-11H-benzo[b]fluorene and pyrene-1-boronic acid was replaced by an intermediate M-d, thereby obtaining 1.96 g of a white solid. The white solid was identified as the compound BH1-3 by analysis according to ASAP-MS (a yield of 65%).

### Synthesis Example 4

### Synthesis of Compound BH1-4

A compound BH1-4 was synthesized through a synthesis pathway below.

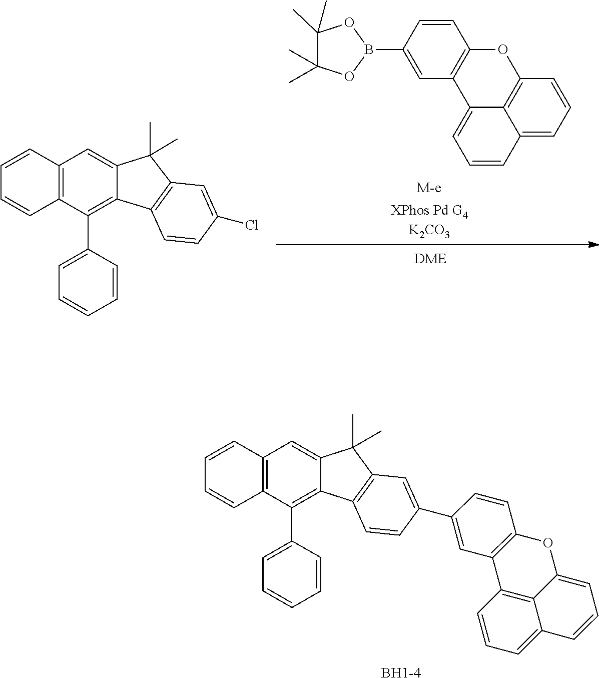 [SMILES: CC1(C)c2cc(Cl)ccc2-c2c1cc1ccccc1c2-c1ccccc1 ; CC1(C)OB(c2ccc3c(c2)-c2cccc4cccc(c24)O3)OC1(C)C ; CC1(C)c2cc(-c3ccc4c(c3)-c3cccc5cccc(c35)O4)ccc2-c2c1cc1ccccc1c2-c1ccccc1 ; "BH1-4"]

### Synthesis of Compound BH1-4

The compound BH1-4 was synthesized in the same manner as the compound BH1-2 except that the intermediate M-c was replaced by 2-chloro-11,11-dimethyl-5-phenyl-11H-benzo[b]fluorene and pyrene-1-boronic acid was replaced by an intermediate M-e, thereby obtaining 2.20 g of a white solid. The white solid was identified as the compound BH1-4 by analysis according to ASAP-MS (a yield of 80%).

What is claimed is:

1. A compound represented by a formula (1) below, 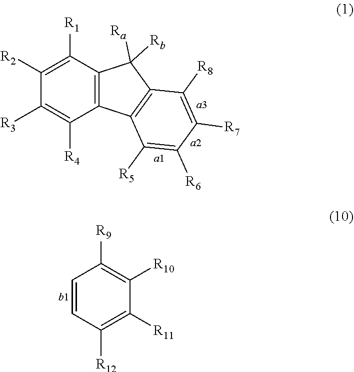
where, in the formula (1):
a combination of Rₐ and R_{b} are mutually bonded to form a substituted or unsubstituted monocyclic ring, mutually bonded to form a substituted or unsubstituted fused ring, or not mutually bonded;
Rₐ and R_{b} forming neither the substituted or unsubstituted monocyclic ring nor the substituted or unsubstituted fused ring are each independently a substituted or unsubstituted alkyl group having 1 to 4 carbon atoms, a substituted or unsubstituted aryl group having 6 to 50 ring carbon atoms, or a substituted or unsubstituted heterocyclic group having 5 to 50 ring atoms;
the heterocyclic group as Rₐ and R_{b} is a heterocyclic group only including at least one atom selected from the group consisting of an oxygen atom, a sulfur atom, and a silicon atom as a hetero atom;
any one combination of a combination of R₅ and R₆, a combination of R₆ and R₇, and a combination of R₇ and R₈ are mutually bonded to form a ring represented by the formula (10);
when a combination of R₅ and R₆ form a ring represented by the formula (10), fused positions are a1 and b1;
when a combination of R₆ and R₇ form a ring represented by the formula (10), fused positions are a2 and b1;
when a combination of R₇ and R₈ form a ring represented by the formula (10), fused positions are a3 and b1;
at least one of R₁ to R₄, R₅ to R₈ not forming a ring represented by the formula (10), or R₉ to R₁₂ in the formula (10) is a substituent Rx;
the substituent Rx is each independently a substituted or unsubstituted aryl group fused with four or more six-membered rings, or a substituted or unsubstituted heterocyclic ring group fused with four or more rings;
the heterocyclic group as the substituent Rx is a heterocyclic group only including at least one atom selected from the group consisting of an oxygen atom, a sulfur atom, and a silicon atom as a hetero atom;
at least one of R₄, R₉, or R₁₂ not being the substituent Rx, or R₅, R₆, or R₈ neither forming a ring represented by the formula (10) nor being the substituent Rx is a substituent Ry;
the substituent Ry is each independently a substituted or unsubstituted phenyl group, a substituted or unsubstituted naphthyl group, a substituted or unsubstituted phenanthryl group, a substituted or unsubstituted dibenzofuranyl group, or a substituted or an unsubstituted dibenzothienyl group;
when the substituent Ry is a substituted 1-naphthyl group, a carbon atom at position 4 of the 1-naphthyl group is unsubstituted;
R₁ to R₄ and R₉ to R₁₂ being neither the substituent Rx nor the substituent Ry, and R₅ to R₈ neither forming a ring represented by the formula (10) nor being the substituent Rx nor being the substituent Ry are each independently a hydrogen atom, a substituted or unsubstituted alkyl group having 1 to 50 carbon atoms, a substituted or unsubstituted cycloalkyl group having 3 to 50 carbon atoms, a substituted or unsubstituted aryl group having 6 to 50 ring carbon atoms, or a substituted or unsubstituted heterocyclic group having 5 to 50 ring atoms;
the heterocyclic group as R₁ to R₁₂ being neither the substituent Rx nor the substituent Ry is a heterocyclic group only including at least one atom selected from the group consisting of an oxygen atom, a sulfur atom, and a silicon atom as a hetero atom; and
a combination of adjacent two or more of R₁ to R₄ and R₉ to R₁₂ being neither the substituent Rx nor the substituent Ry, and R₅ to R₈ neither forming a ring represented by the formula (10) nor being the substituent Rx nor being the substituent Ry are not bonded to each other to form no ring.

2. The compound according to claim 1, wherein at least one of R₂ or R₃ is the substituent Rx.

3. The compound according to claim 1, wherein at least one of R₄, R₅, R₉, or R₁₂ is the substituent Ry.

4. The compound according to claim 1, wherein a combination of Rₐ and R_{b} are not bonded to each other to form no ring.

5. The compound according to claim 1, wherein R^{a} and R_{b} are each independently a substituted or unsubstituted alkyl group having 1 to 4 carbon atoms.

6. The compound according to claim 1, wherein
the substituent Rx is each independently a substituted or unsubstituted pyrenyl group, a substituted or unsubstituted benzanthryl group, a substituted or unsubstituted benzoxanthenyl group, or a substituted or unsubstituted naphthobenzofuranyl group,
the pyrenyl group is represented by a formula (Rx-1) below,
the benzanthryl group is represented by a formula (Rx-2) below,
the benzoxanthenyl group is represented by a formula (Rx-3) below, and
the naphthobenzofuranyl group is represented by a formula (Rx-4) below, 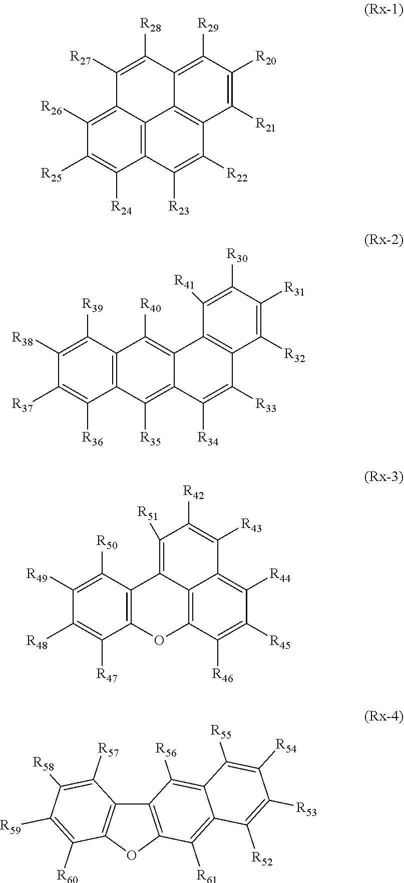
where, in the formula (Rx-1), one of R₂₀ to R₂₉ is a bond of a substituent Rx, and R₂₀ to R₂₉ other than the bond are each independently a hydrogen atom or a substituent,
in the formula (Rx-2), one of R₃₀ to R₄₁ is a bond of a substituent Rx, and R₃₀ to R₄₁ other than the bond are each independently a hydrogen atom or a substituent,
in the formula (Rx-3), one of R₄₂ to R₅₁ is a bond of a substituent Rx, and R₄₂ to R₅₁ other than the bond are each independently a hydrogen atom or a substituent, and
in the formula (Rx-4), one of R₅₂ to R₆₁ is a bond of a substituent Rx, and R₅₂ to R₆₁ other than the bond are each independently a hydrogen atom or a substituent.

7. The compound according to claim 1, wherein the compound represented by the formula (1) is a compound represented by a formula (1A) below, 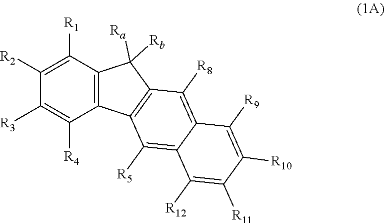
where, in the formula (1A): Rₐ, R_{b}, R₁ to R₅, and R₈ each represent the same as Rₐ, R_{b}, R₁ to R₅, and R₈ in the formula (1); and R₉ to R₁₂ each independently represent the same as R₉ to R₁₂ in the formula (10).

8. The compound according to claim 7, wherein R₂ is the substituent Rx, and R₄ or R₅ is the substituent Ry.

9. The compound according to claim 7, wherein R^{a} and R_{b} are each independently a substituted or unsubstituted alkyl group having 1 to 4 carbon atoms.

10. The compound according to claim 7, wherein R₁ to R₅ and R₈ to R₁₂ being neither the substituent Rx nor the substituent Ry are each a hydrogen atom.

11. The compound according to claim 7, wherein the compound represented by the formula (1A) is a compound represented by a formula (1A-A), (1A-B), (1A-C), or (1A-D) below, 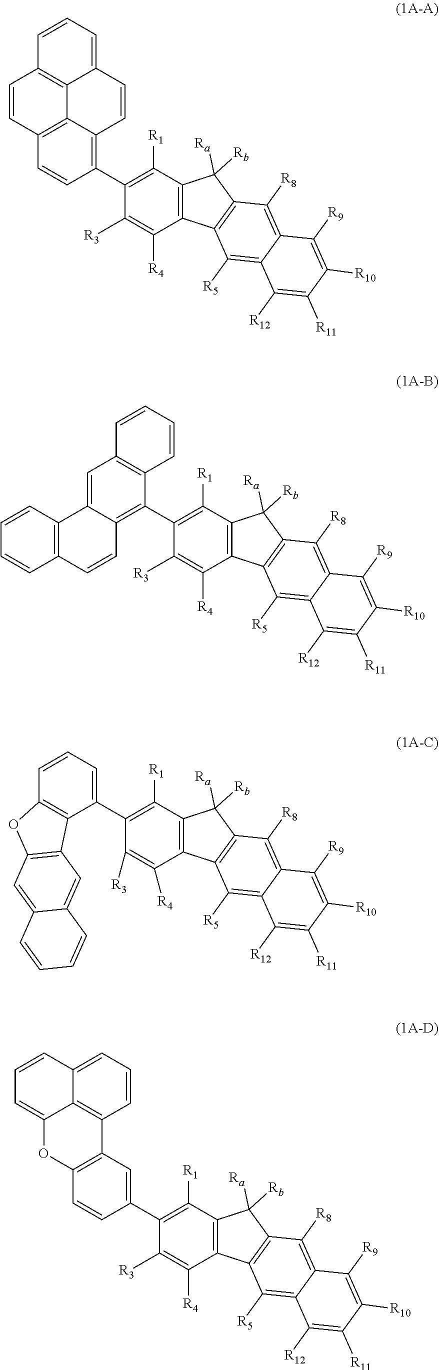
where, in the formulae (1A-A), (1A-B), (1A-C), and (1A-D):
R₄ or R₅ is each independently the substituent Ry; Rₐ, R_{b}, R₁, R₃, and R₈ represent each independently the same as Rₐ, R_{b}, R₁, R₃, and R₈ in the formula (1); and R₉ to R₁₂ each independently represent the same as R₉ to R₁₂ in the formula (10).

12. The compound according to claim 1, wherein the compound represented by the formula (1) is a compound represented by a formula (1B) below, 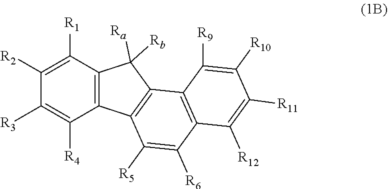
where, in the formula (1B): Rₐ, R_{b} and R₁ to R₆ each independently represent the same as Rₐ, R_{b} and R₁ to R₆ in the formula (1); and R₉ to R₁₂ each independently represent the same as R₉ to R₁₂ in the formula (10).

13. The compound according to claim 7, wherein R₃ is the substituent Rx, and R₁₂ is the substituent Ry.

14. The compound according to claim 12, wherein Rₐ and R_{b} are each independently a substituted or unsubstituted alkyl group having 1 to 4 carbon atoms.

15. The compound according to claim 12, wherein R₁ to R₆ and R₉ to R₁₂ being neither the substituent Rx nor the substituent Ry are each a hydrogen atom.

16. The compound according to claim 12, wherein the compound represented by the formula (1B) is a compound represented by a formula (1B-A), (1B-B), (1B-C), or (1B-D) below, 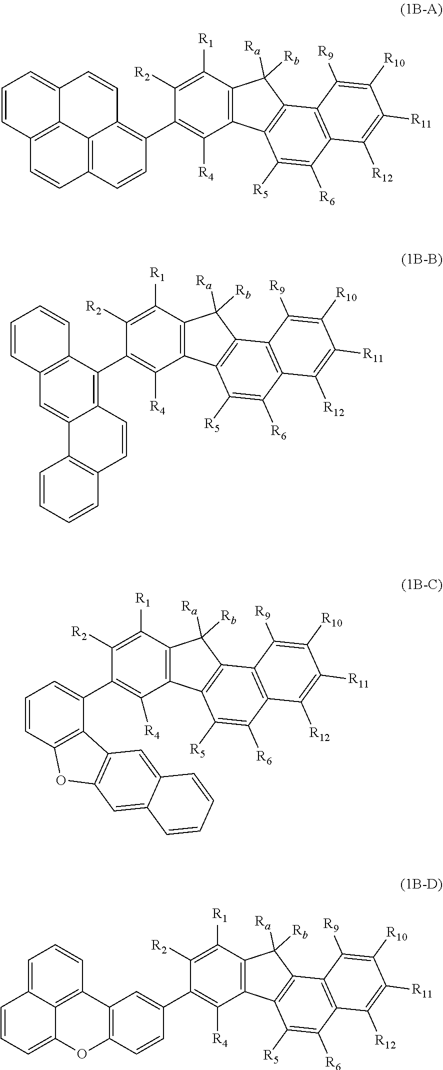
where, in the formulae (1B-A), (1B-B), (1B-C), and (1B-D): R₁₂ is the substituent Ry; Rₐ, R_{b}, R₁, R₂, and R₄ to Re each independently represent the same as Rₐ, R_{b}, R₁, R₂, and R₄ to R₆ in the formula (1); and R₉ to R₁₁ each independently represent the same as R₉ to R₁₁ in the formula (10).

17. The compound according to claim 1, wherein the compound represented by the formula (1) is a compound represented by a formula (1C) below, 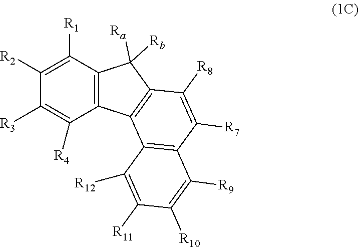
where, in the formula (1C): Rₐ, R_{b}, R₁ to R₄, R₇, and R₈ each independently represent the same as Rₐ, R_{b}, R₁ to R₄, R₇, and R₈ in the formula (1); and Ry to R₁₂ each independently represent the same as R₉ to R₁₂ in the formula (10).

18. The compound according to claim 17, wherein R₂ is the substituent Rx, and R₉ is the substituent Ry.

19. The compound according to claim 17, wherein Rₐ and R_{b} are each independently a substituted or unsubstituted alkyl group having 1 to 4 carbon atoms.

20. The compound according to claim 17, wherein R₁ to R₄ and R₇ to R₁₂ being neither the substituent Rx nor the substituent Ry are each a hydrogen atom.

21. The compound according to claim 17, wherein the compound represented by the formula (1C) is a compound represented by a formula (1C-A), (1C-B), (1C-C), or (1C-D) below, 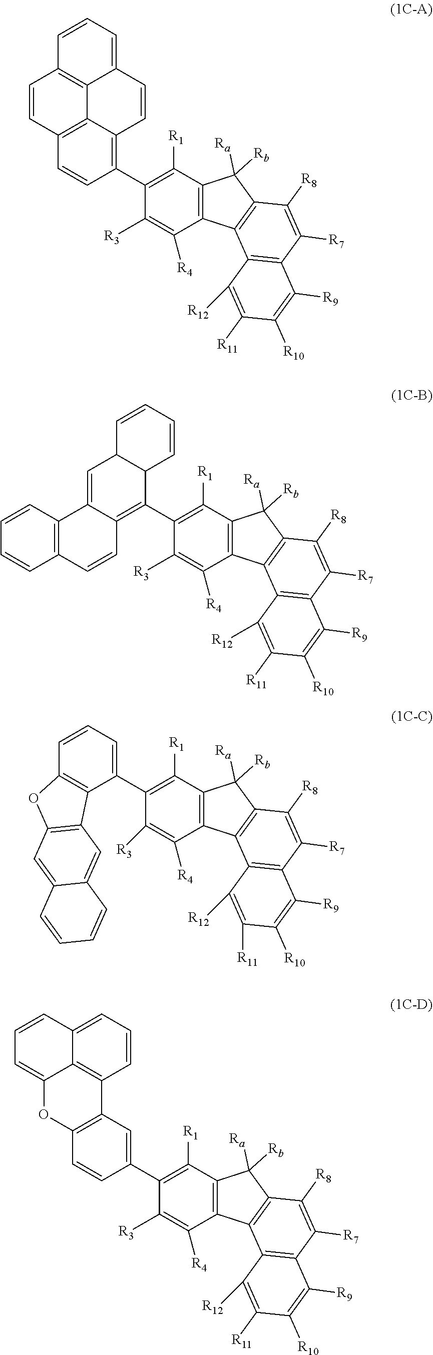
where, in the formulae (1C-A), (1C-B), (1C-C), and (1C-D): R₉ is the substituent Ry; Rₐ, R_{b}, R₁, R₃, R₄, R₇ and R₈ each independently represent the same as Rₐ, R_{b}, R₁, R₃, R₄, R₇ and R₈ in the formula (1); and R₁₀ to R₁₂ each independently represent the same as R₁₀ to R₁₂ in the formula (10).

22. An organic electroluminescence device comprising: the compound according to claim 1 as a first compound.

23. The organic electroluminescence device according to claim 22, comprising:
an anode;
a cathode; and
an organic layer disposed between the anode and the cathode, wherein
at least one layer of the organic layer comprises the first compound.

24. The organic electroluminescence device according to claim 23, wherein
the organic layer comprises an emitting region,
the emitting region comprises a first emitting layer and a second emitting layer, and
the first emitting layer comprises the first compound as a first host material.

25. The organic electroluminescence device according to claim 24, wherein
the first emitting layer comprises a first luminescent compound, and
the first luminescent compound emits light having a maximum peak wavelength of 500 nm or less.

26. The organic electroluminescence device according to claim 24, wherein
the second emitting layer comprises a second host material, and
a triplet energy of the first host material T₁(H1) and a triplet energy of the second host material T₁(H2) satisfy a relationship of a numerical formula (Numerical Formula 1) below,
$${T}_{1} ( H 1 ) > {T}_{1} ( H 2 ) . \qquad ( Numerical Formula 1 )$$

27. The organic electroluminescence device according to claim 24, further comprising: a hole transporting layer between the anode and the emitting region.

28. The organic electroluminescence device according to claim 24, further comprising: an electron transporting layer between the cathode and the emitting region.

29. An electronic device comprising the organic electroluminescence device according to claim 23.
